(19) United States
(12) Patent Application Publication
(10) Pub. No.: US 2025/0011299 A1
(43) Pub. Date: Jan. 9, 2025

(54) PYRIDAZINYL AMINO DERIVATIVES AS ALK5 INHIBITORS

(71) Applicant: CHIESI FARMACEUTICI S.P.A., Parma (IT)

(72) Inventors: Daniela PIZZIRANI, Parma (IT); Paolo RONCHI, Parma (IT); Sara GUARIENTO, Parma (IT); Daniele PALA, Parma (IT); Paolo BRUNO, Parma (IT); Teresa SEMERARO, Parma (IT); Donatella RESCIGNO, Parma (IT)

(73) Assignee: CHIESI FARMACEUTICI S.P.A., Parma (IT)

(21) Appl. No.: 18/693,564

(22) PCT Filed: Sep. 20, 2022
(86) PCT No.: PCT/EP2022/076130

§ 371 Date: Mar. 20, 2024

(30) Foreign Application Priority Data
Sep. 21, 2021 (EP) 21198025.5
Dec. 21, 2021 (EP) 21216519.5
Sep. 13, 2022 (PK) 598/2022

(51) Int. Cl. C07D 401/12; A61K 31/501; A61K 31/506; A61K 31/5377; A61K 31/541; A61K 31/551; C07D 401/14; C07D 403/12; C07D 405/14; C07D 409/14; C07D 471/04; C07D 471/10; C07D 487/08; C07D 487/10; C07D 487/18; C07D 491/107

(52) U.S. Cl. CPC C07D 401/12; A61K 31/501; A61K 31/506; A61K 31/5377; A61K 31/541; A61K 31/551; C07D 401/14; C07D 403/12; C07D 405/14; C07D 409/14; C07D 471/04; C07D 471/10; C07D 487/08; C07D 487/10; C07D 487/18; C07D 491/107

(57) ABSTRACT

The present invention relates to a compound of general formula (I) inhibiting the transforming growth factor-β (TGF-β) type I receptor (ALK5), methods of preparing such compounds, pharmaceutical compositions containing them and therapeutic use thereof. The compounds of the invention may be useful in the treatment of diseases or conditions associated with a dysregulation of ALK5 signaling pathway in a mammal.

### FIELD OF THE INVENTION

The present invention generally relates to compounds inhibiting the transforming growth factor β (TGF β) type I receptor (ALK5) (hereinafter ALK5 inhibitors), methods of preparing such compounds, pharmaceutical compositions containing them and therapeutic use thereof, the compounds of the invention may be useful for instance in the treatment of many disease, disorder, or condition associated with ALK5 signaling pathway.

### BACKGROUND OF THE INVENTION

The Transforming Growth Factor β (TGF β) is a protein belonging to the TGF R superfamily. It is involved in several processes, both cellular, such as proliferation, migration and differentiation, and biological, including wound healing, immunesuppression, cancerogenesis and extracellular matrix production.

The TGF β superfamily also includes, among others, other members known as activins (Acts) (see e.g. Hinck A P, FEBS Letters 586 (2012); 1860-1870). The binding of the peptide initiates the TGF β signalling cascade through the formation of a heterotetrameric complex composed of two different serine/threonine kinases receptors: type 1 (TGFβR1/ALK5) and type 2 (TGFβR2). TGFβR1/ALK5 is recruited and activated through the phosphorylation of its intracellular domain by TGFβR2, leading in turn to the phosphorylation of the receptor-activated (R)-Smad family, resulting in the activation of target gene transcription (see e.g. Sheppard D., Proc Am Thorac Soc. (2006); (3):413-417). Similarly to the TGF β signaling, the type I receptor for activin, ALK4, leads to the activation of target gene transcription (see e.g. Heldin C H et al., Cold Spring Harb Perspect Biol. (2016) Aug. 1; 8 (8)). Several studies have linked an excessive and/or dysregulated TGF β activity with many diseases including cancer and fibrosis (see e.g. Syed V, J Cell Biochem. (2016) Jun.; 117(6):1279-87; Jakowlew S B. Cancer Metastasis Rev. (2006) Sep.; 25(3):435-57). Among fibrotic disorders, a crucial role of TGFβ has been shown in organs such as lung, heart, liver, and kidney (see e.g. Alhamad E H, J Thorac Dis. (2015); 7(3):386-93). In particular, TGFβ expression is increased in fibrotic lung diseases, such as idiopathic pulmonary fibrosis (IPF), and in chronic inflammatory conditions, such as chronic obstructive pulmonary disease and asthma (see e.g. Thomas B J et al., Am J Respir Cell Mol Biol. (2016); (55):759-766). In lung, TGFβ is expressed in several cell types, like epithelial cells, endothelial cells, connective tissue cells, macrophages and fibroblasts. These cell populations may produce excess of TGFβ in IPF human lung tissue. Moreover, high levels of TGFβ have been detected in lung tissue and BAL of IPF patients (see e.g. Bergeron A et al., Eur Respir J (2003); 22:69-76). TGFβ gene expression and TGFβ protein production have been observed to increase in a variety of animal models of pulmonary fibrosis caused by bleomycin, silica, asbestos, and radiation (see e.g. Wei F et al., Int Immunopharmacol. (2017) Jul.; 48:67-75; Choe J Y et al., Inflamm Res. (2010) Mar.; 59(3):177-88; Wang X et al., Respir Res (2009); 10, 36) and it has also been reported how the TGFβ expression is sufficient to induce progressive fibrosis in rodents (see e.g. Sime P J et al., J Clin Invest (1997); 100:768-776; Kim K K et al.). Contrarily, TGFβ signalling inhibition obtained by employing knockout (KO) animals can inhibit fibrosis development through TGFβ-linked mechanisms (see e.g. Bonniaud P et al., Am J Respir Crit Care Med (2005); 171:889-898; 34). Similar results have been achieved with inhibition of TGFβR1 in mouse bleomycin disease model (see e.g. Wei Y et al., J Clin Invest. (2017); 127(10):3675-3688). Activin signalling dysregulation, similarly to TGFβ, is associated to fibroblasts proliferation, myofibroblasts differentiation and accumulation of extracellular matrix (ECM) (see e.g. Yamashita et al., J. Am. Soc. Nephrol. (2004) 15, 91-101). Moreover, overexpression of activin has been linked to pathological conditions and fibrosis development in different organs, such as liver (see e.g. Patella et al., Am. J. Physiol. Gastrointest. Liver Physiol. (2006) 290, G137-G144), kidney (see e.g. Agapova et al., Kidney Int. (2016) 89, 1231-1243), heart (see e.g. Yndestad et al., Circulation (2004) 109, 1379-1385), and lung (see e.g. de Kretser et al., Crit. Care (2013) 17:R263). Taken together these data suggest the importance of targeting ALK5 to treat pharmacologically the aforementioned diseases, linked to the dysregulated TGF signaling pathway. The TGFβ signaling is strongly involved in the cardiovascular homeostasis (see e.g. van Meeteren L A et al., Springer (2013)). Several studies in humans and mice have shown the main role of TGFβ in angiogenesis and vascular morphogenesis. Moreover, TGFβ plays a key role in the development and functionality of cardiac valves. It is therefore clear the importance of a selective regulation of TGFβ pathway to target the pathological effects avoiding the suppression of the signaling needed for a correct homeostasis.

The answer to this crucial point could be addressed by using the inhalation route to deliver an antiTGFβ drug. The inhalatory route would allow the treatment of the affected lung compartment bypassing the issue of the heart exposure.

Various compounds have been described in the literature as ALK5 and/or ALK4 receptor inhibitors.

WO2008/006583, WO2009/087212, WO2009/087224, WO2009/087225, WO2009/133070, WO2009/013335 and WO2009/050183 (Novartis) disclose respectively pyrimidine, pyridine, imidazo pyridine, pyrrolo pyrimidine and pyrrolo pyridine, imidazo pyridazine, imidazo pyridine derivatives useful for the treatment of ALK4- or ALK5-mediated diseases such as inflammatory or obstructive airways diseases, pulmonary hypertension and pulmonary fibrosis.

WO00/61576 and US2003/0149277 (Smithkline Beecham Corp) disclose triarylimidazole derivatives as ALK5 inhibitors useful for the treatment of, among others, renal disease, wound healing, kidney disease, congestive heart failure, ulcers, impaired neurological function and any disease wherein fibrosis is a major component.

WO01/62756 (Smithkline Beecham P.L.C.) discloses pyridinylimidazole derivatives as ALK5 inhibitors useful for the treatment of, among others, renal disease, wound healing, kidney disease, congestive heart failure, ulcers, impaired neurological function and any disease wherein fibrosis is a major component.

WO03/087304 (Biogen Inc.) discloses tri-substituted heteroaryls as ALK5 and/or ALK4 inhibitors useful for the treatment of, among others, idiopathic pulmonary fibrosis, diabetic nephropathy, hepatic fibrosis, pulmonary fibrosis, acute lung injury, post-infarction cardiac fibrosis, fibrotic cancers and fibroma.

Pyridazinyl amino derivatives have been disclosed in the literature, but not as ALK5 inhibitors.

WO2005/033105 (Amgen) discloses, among other compounds, pyridazinyl amino derivatives as vanilloid receptor ligands, for the treatment of a large number of diseases and disordes, not including fibrosis.

WO2002/022605 and WO2002/022602 (Vertex) describe, among others, pyridazine compounds as protein kinase inhibitors useful for the treatment of cancer, diabetes, Alzheimer's disease and schizophrenia.

WO02/24681 (Ortho-McNeil Pharmaceutical Inc.) describes pyridazine compounds as tyrosine kinase inhibitors useful as anti-tumor agents, and to treat diabetic retinopathy, rheumatoid arthritis, endometriosis and psoriasis.

Of note, inhibition of ALK5 receptor may be useful for the treatment of fibrosis and disease, disorder and conditions that result from fibrosis.

Several efforts have been done in the past years to develop novel ALK5 receptor inhibitors useful for the treatment of several diseases and some of those compounds have shown efficacy also in humans.

However, there remains a potential for developing inhibitors of receptors ALK5 characterized by good potency, useful for the treatment of diseases or conditions associated with a dysregulation of ALK5 signaling pathway, in particular fibrosis.

In particular, there remains a potential for developing inhibitors of receptors ALK5 useful for the treatment of diseases or conditions associated with a dysregulation of ALK5 signaling in the respiratory field, in particular idiopathic pulmonary fibrosis (IPF), to be administered by the inhalation route and characterized by a good inhalatory profile, that corresponds to a good activity on the lung, a good lung retention and to a low metabolic stability in order to minimize the systemic exposure and correlated safety issues.

In this direction, we have surprisingly found a new series of compounds of general formula (I) that solves the problem of providing potent inhibitors of ALK5 receptor for administration by inhalation, that shows, at the same time, a good inhalatory profile, low metabolic stability, low systemic exposure, improved safety and tolerability, and a good selectivity across the kinome.

### SUMMARY OF THE INVENTION

In a first aspect the present invention relates to compounds of formula (I)

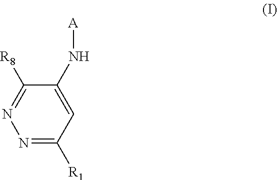

wherein
A is selected from the groups consisting of A1, A2, A3 and A4

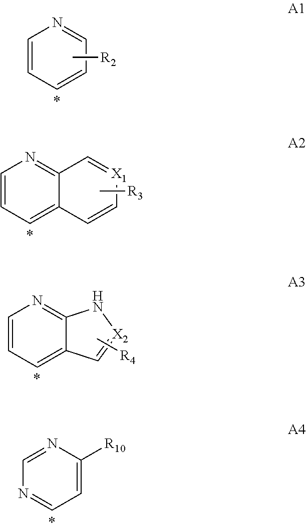

R₁ is selected from the group consisting of aryl and pyridyl, wherein said aryl and pyridyl are optionally substituted by one or more groups selected from halogen atoms and —(C₁-C₆)alkyl;
R₂ is selected from the group consisting of —NR₅C(O)R₆, —NR₅R₉ and —NH₂;
X₁ is C or CH;
X₂ is C, CH or N;
R₃ is —OR₇;
R₄ is H or —C(O)O—(C₁-C₆)alkyl;
R₅ is H or —(C₁-C₆)alkyl;
R₆ is selected from the group consisting of —(C₃-C₉)heterocycloalkyl substituted by one or more —(C₁-C₆)alkyl; —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl and —(C₃-C₆)cycloalkyl; —(C₁-C₆)alkylene-NH₂; —(C₃-C₆)cycloalkyl optionally substituted by one or more —(C₁₋C₆)alkylene-(C₃-C₉)heterocycloalkyl, wherein said —(C₃-C₉)heterocycloalkyl is optionally substituted by one or more groups selected from —(C₁-C₆)alkyl and —(C₃-C₆)cycloalkyl; and —(C₃-C₆)cycloalkyl optionally substituted by one or more —(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —O—(C₁-C₆)alkyl, —C(O)OH, —C(O)O—(C₁-C₆)alkyl, —(C₁-C₆)haloalkyl, —(C₃-C₆)cycloalkyl and halogen atoms;
R₇ is selected from the group consisting of —(C₁-C₆)alkyl and —(C₁-C₆)alkylene-(C₃-C₉)heterocycloalkyl, wherein said —(C₃-C₉)heterocycloalkyl is optionally substituted by one or more —(C₁-C₆)alkyl;
R₈ is selected from the group consisting of —NR_{A}R_{B}; —SH; —S—(C₁-C₆)alkyl, wherein said —(C₁-C₆)alkyl is optionally substituted by one or more —OH; —S—(C₁-C₆)alkylene-OH; —S—(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S(O)=NH—(C₁-C₆)alkyl; —S(O)₂—(C₁-C₆)alkyl; —S(O)—(C₁-C₆)alkyl; —S—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH and —OH; S—(C₁-C₆)alkylene-aryl, wherein said aryl is optionally substituted by one or more groups selected from —C(O)OH, —C(O)O—(C₁-C₆)alkylene-NR_{A}R_{C} and —C(O)O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-Si((C₁-C₆)alkyl)₃; —S—(C₁-C₆)alkylene-O—(C₁-C₆)alkylene-OH; —S—(C₁-C₆)alkylene-O—(C₁-C₆)alkylene-(C₃-C₉)heterocycloalkyl, wherein said —(C₃-C₉)heterocycloalkyl is optionally substituted by one or more groups selected from oxo and —(C₁-C₆)alkyl; —S—(C₁-C₆)alkylene-NH—C(O)—(C₃-C₉)heterocycloalkyl, wherein said —(C₃-C₉)heterocycloalkyl is optionally substituted by one or more oxo; —S—(C₁-C₆)alkylene-NH—(C₃-C₉)heterocycloalkyl, wherein said —(C₃-C₉)heterocycloalkyl is optionally substituted by one or more oxo; —O—(C₁-C₆)alkyl; —O—(C₁-C₆)haloalkyl; —O—(C₁-C₆)alkylene-OH, wherein said —O—(C₁-C₆)alkylene is substituted by one or more —OH; —O—(C₁-C₆)alkylene-C(O)O—(C₁-C₆)alkyl; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁-C₆)alkylene-S(O)—(C₁-C₆)alkyl; —O—(C₁-C₆)alkylene-S(O)₂—(C₁-C₆)alkyl; —O—(C₁-C₆)alkylene-NH—S(O)₂—(C₁-C₆)alkyl; —O—(C₁-C₆)alkylene-O—(C₁-C₆)alkyl; —O—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH, —C(O)O—(C₁-C₆)alkyl and —OH; —O—(C₃-C₆)cycloalkyl, wherein said —(C₃-C₆)cycloalkyl is optionally substituted by one or more groups selected from —(C₁-C₆)alkylene-OH and —OH; —O—(C₁-C₆)alkylene-aryl, wherein said aryl is optionally substituted by one or more —OH; —O—(C₁-C₆)alkylene-aryl, wherein said aryl is fused to a —(C₅-C₆)heterocycloalkyl, wherein said —(C₅-C₆)heterocycloalkyl is optionally substitued by one or more groups selected from oxo and —(C₁-C₆)alkyl; —O—(C₃-C₉)heterocycloalkyl; and —O—(C₁-C₆)alkylene-(C₃—C₉)heterocycloalkyl, wherein said —(C₃-C₉)heterocycloalkyl is optionally substituted by one or more groups selected from —(C₁-C₆)alkyl and oxo;
R₉ is a heteroaryl optionally substituted by one or more groups selected from —C(O)O—(C₁-C₆)alkyl and —(C₃-C₉)heterocycloalkyl, wherein said —(C₃-C₉)heterocycloalkyl is optionally substituted by one or more —(C₁-C₆)alkyl;
R₁₀ is —NR₅C(O)R₆;
R_{A} is H or —(C₁-C₆)alkyl;
R_{B} is H or selected from the group consisting of —(C₁-C₆)alkyl optionally substituted by one or more groups selected from halogen and —OH; —S(O)₂—(C₁-C₆)alkyl; —(C₁-C₆)alkylene-aryl, wherein said aryl is susbtituted by —OH; —(C₁-C₆)alkylene-OH; —(C₃-C₉)heterocycloalkyl; —(C₁-C₆)alkylene-C(O)O—(C₁-C₆)alkyl; —(C₁-C₆)alkylene-aryl-OCO—(C₁-C₆)alkyl; and —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —(C₁-C₆)alkylene-O—(C₁-C₆)alkyl, —(C₁-C₆)haloalkyl and oxo; or alternatively R_{A} and R_{B} together with the nitrogen atom to which they are attached may form a —(C₃-C₆)heterocycloalkyl, wherein said —(C₃-C₆)heterocycloalkyl is optionally substituted by one or more groups selected from —C(O)OH, —(C₁-C₆)alkylene-OH, —C(O)O—(C₁-C₆)alkyl and oxo, or said —(C₃-C₆)heterocycloalkyl is optionally substituted on two adjacent carbon atoms forming an additional condensed —(C₅-C₆)heterocycloalkyl, optionally substituted by oxo;
R_{C} is —(C₁-C₆)alkyl; and pharmaceutically acceptable salts thereof.

In a second aspect, the invention refers to a pharmaceutical composition comprising a compound of formula (I) and pharmaceutically acceptable salts thereof in admixture with one or more pharmaceutically acceptable carrier or excipient.

In a third aspect, the invention refers to a compound of formula (I) and pharmaceutically acceptable salts, or to a pharmaceutical composition comprising a compound of formula (I) and pharmaceutically acceptable salts thereof, for use as a medicament.

In a further aspect, the invention refers to a compound of formula (I) and pharmaceutically acceptable salts thereof, or to a pharmaceutical composition comprising a compound of formula (I) and pharmaceutically acceptable salts thereof, for use in preventing and/or treating a disease, disorder or condition mediated by ALK5 receptor in a mammal.

In a further aspect, the invention refers to a compound of formula (I) and pharmaceutically acceptable salts thereof, or to a pharmaceutical composition comprising a compound of formula (I) and pharmaceutically acceptable salts thereof, for use in the prevention and/or treatment of fibrosis and/or diseases, disorders, or conditions that involve fibrosis.

In a further aspect, the invention refers to a compound of formula (I) and pharmaceutically acceptable salts thereof, or to a pharmaceutical composition comprising a compound of formula (I) and pharmaceutically acceptable salts thereof, for use in the prevention and/or treatment idiopathic pulmonary fibrosis (IPF).

### DETAILED DESCRIPTION OF THE INVENTION

### Definitions

Unless otherwise specified, the compound of formula (I) of the present invention is intended to include also stereoisomers, tautomers or pharmaceutically acceptable salts or solvates thereof.

Unless otherwise specified, the compound of formula (I) of the present invention is intended to include also the compounds of formula (Ia), (Iaa), (Ib), (Iba), (Ic), (Ica) and (Id).

The term “pharmaceutically acceptable salts”, as used herein, refers to derivatives of compounds of formula (I) wherein the parent compound is suitably modified by converting any of the free acid or basic group, if present, into the corresponding addition salt with any base or acid conventionally intended as being pharmaceutically acceptable.

Suitable examples of said salts may thus include mineral or organic acid addition salts of basic residues such as amino groups, as well as mineral or organic basic addition salts of acid residues such as carboxylic groups.

Cations of inorganic bases which can be suitably used to prepare salts comprise ions of alkali or alkaline earth metals such as potassium, sodium, calcium or magnesium.

Those obtained by reacting the main compound, functioning as a base, with an inorganic or organic acid to form a salt comprise, for example, salts of hydrochloric acid, hydrobromic acid, sulfuric acid, phosphoric acid, methane sulfonic acid, camphor sulfonic acid, acetic acid, oxalic acid, maleic acid, fumaric acid, succinic acid and citric acid.

The term “solvate” means a physical association of a compound of this invention with one or more solvent molecules, whether organic or inorganic. This physical association includes hydrogen bonding. In certain instances, the solvate will be capable of isolation, for example, when one or more solvent molecules are incorporated in the crystal lattice of the crystalline solid. The solvate may comprise either a stoichiometric or nonstoichiometric amount of the solvent molecules.

The term “stereoisomer” refers to isomers of identical constitution that differ in the arrangement of their atoms in space. Enantiomers and diastereomers are examples of stereoisomers.

The term “enantiomer” refers to one of a pair of molecular species that are mirror images of each other and are not superimposable.

The term “diastereomer” refers to stereoisomers that are not mirror images.

The term “racemate” or “racemic mixture” refers to a composition composed of equimolar quantities of two enantiomeric species, wherein the composition is devoid of optical activity.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUP AC Recommendations 1996, Pure and Applied Chemistry, 68:2193-2222 (1996)).

The term “tautomer” refers to each of two or more isomers of a compound that exist together in equilibrium and are readily interchanged by migration of an atom or group within the molecule.

The term “halogen” or “halogen atoms” or “halo” as used herein includes fluorine, chlorine, bromine, and iodine atom.

The term “(Cₓ-C_{y})alkyl” wherein x and y are integers, refers to a straight or branched chain alkyl group having from x to y carbon atoms. Thus, when x is 1 and y is 6, for example, the term includes methyl, ethyl, n-propyl, isopropyl, n-butyl, isobutyl, sec-butyl, t-butyl, n-pentyl and n-hexyl.

The term “(Cₓ-C_{y})alkoxy” wherein x and y are integers, refers to a straight or branched hydrocarbon of the indicated number of carbons, linked to the rest of the molecule through an oxygen bridge.

The term “(Cₓ-C_{y})alkylene” wherein x and y are integers, refers to a (Cₓ-C_{y})alkyl radical having in total two unsatisfied valencies, such as a divalent methylene radical.

The expressions “(Cₓ-C_{y})haloalkyl” wherein x and y are integers, refer to the above defined “(Cₓ-C_{y})alkyl” groups wherein one or more hydrogen atoms are replaced by one or more halogen atoms, which can be the same or different. Examples of said “(Cₓ-C_{y})haloalkyl” groups may thus include halogenated, poly-halogenated and fully halogenated alkyl groups wherein all hydrogen atoms are replaced by halogen atoms, e.g. trifluoromethyl.

The term “(Cₓ-C_{y})cycloalkyl” wherein x and y are integers, refers to saturated cyclic hydrocarbon groups containing the indicated number of ring carbon atoms. Examples include cyclopropyl, cyclobutyl, cyclopentyl, cyclohexyl, cycloheptyl.

The term “aryl” refers to mono cyclic carbon ring systems which have 6 ring atoms wherein the ring is aromatic. Examples of suitable aryl monocyclic ring systems include, for instance, phenyl.

The term “heteroaryl” refers to a mono- or bi-cyclic aromatic group containing one or more heteroatoms selected from S, N and O, and includes groups having two such monocyclic rings, or one such monocyclic ring and one monocyclic aryl ring, which are fused through a common bond.

The term “(Cₓ-C_{y})heterocycloalkyl” wherein x and y are integers, refers to saturated or partially unsaturated monocyclic (Cₓ-C_{y})cycloalkyl groups in which at least one ring carbon atom is replaced by at least one heteroatom (e.g. N, S or O) or may bear an -oxo (=O) substituent group. Said heterocycloalkyl may be further optionally substituted on the available positions in the ring, namely on a carbon atom, or on a heteroatom available for substitution. Substitution may be on a carbon atom including spiro disubstitution, forming bicyclic system where two “(Cₓ-C_{y})heterocycloalkyl rings, or one (Cₓ-C_{y})heterocycloalkyl and one (Cₓ-C_{y})cycloalkyl ring, are connected through a single carbon atom. Substitution may be as well as on two adjacent carbon atoms forming an additional condensed 5 to 6 membered heterocycloalkyl ring. Examples of spiro rings comprise and are not limited to, for examples, 6-methyl-2,6-diazaspiro[3.3]heptan-2-yl and 2-methyl-2,8-diazaspiro[4.5]decane; examples of condensed rings include, for instance, 2,2-dimethyl-2H-1,3-benzodioxol-5-yl. Moreover, said heterocycloalkyl may be a diazabicyclo ring or a cyclic carbonate. Examples of diazabicyclo ring include and are not limited to, for instance, 5-methyl-2,5-diazabicyclo[2.2.1]heptan-2-yl and 6-methyl-3,6-diazabicyclo[3.2.2]nonan-3-yl; examples of suitable cyclic carbonates include, for instance, 1,3-dioxalan-2-one and 4-methyl-1,3-dioxol-2-one.

Throughout the specification the use of an asterisk “*” in the definition of a structural formula, indicates the point of attachment for the radical group to the rest of the molecule.

A dash (“-”) that is not between two letters or symbols is meant to represent the point of attachment for a substituent.

The carbonyl group is herein preferably represented as —C(O)— as an alternative to the other common representations such as —CO—, —(CO)— or —C(═O)—

In general, the bracketed group is a lateral group, not included into the chain, and brackets are used, when deemed useful, to help disambiguating linear chemical formulas; e.g. the sulfonyl group —SO₂— might be also represented as —S(O)₂— to disambiguate e.g. with respect to the sulfinic group —S(O)O—.

The present invention relates to novel compounds differing from the structures disclosed in the art at least for a common new core scaffold. In fact the invention relates to compounds that are [pyridazin-4-yl]amino derivatives, which are inhibitors of receptor ALK5 that have therapeutically desirable characteristics, particularly promising for some fibrosis, including idiopathic pulmonary fibrosis (IPF).

The compounds of the invention are active as inhibitors of ALK5 receptor, they are potent and show improved properties such as a good inhalatory profile, a low metabolic stability, a low systemic exposure, improved safety and tolerability, and a good selectivity across the kinome. In this respect, the state of the art does not describe or suggest pyridazinyl amino derivatives of general formula (I) of the present invention having an inhibitory activity on receptor ALK5 which represents a solution to the aforementioned need.

Amgen discloses, among other compounds, pyridazinyl amino derivatives. The compounds of formula (I) of the present invention differ from the Amgen ones at least for the substituents on rings A1, A2 and A3. Amgen discloses compounds as vanilloid receptor ligands for the treatment of a large number of diseases and disordes. Amgen neither discloses compounds as ALK5 inhibitors, nor compounds for the treatment of fibrosis.

Vertex describes, among others, pyridazine derivatives. The compounds of formula (I) of the present invention differ from the Vertex ones at least for the presence of a pyridyl or pyridyl condensed group linked to the amino linker bearing the pyridazine ring, instead of a triazole group. Vertex compounds are described as protein kinase inhibitors useful for the treatment of cancer, diabetes, Alzheimer's disease and schizophrenia. Vertex neither describes compounds as ALK5 inhibitors, nor for the treatment of fibrosis.

Ortho-McNeil describes pyridazine compounds. The compounds of formula (I) of the present invention differ from the Ortho-McNeil ones at least for the position of the two nitrogen atoms in the pyridazine ring. Ortho-McNeil compounds are described as tyrosine kinase inhibitors useful as anti-tumor agents, and to treat diabetic retinopathy, rheumatoid arthritis, endometriosis and psoriasis. Ortho-McNeil neither discloses compounds as ALK5 inhibitors, nor compounds for the treatment of fibrosis.

In more details, the present invention refers to a series of compounds represented by the general formula (I) as herein below described in details, which are endowed with an inhhibitory activity on receptor ALK5 receptor. Advantageously, the inhibitory action on receptor ALK5 can be effective in the treatment of those diseases where these receptors play a relevant role in the pathogenesis such as fibrosis and disease, disorder and condition from fibrosis.

Differently from similar compounds of the prior art, the compounds of formula (I) of the present invention are able to act as antagonists of ALK5 receptor, particularly appreciated by the skilled person when looking at a suitable and efficacious compounds useful for the treatment of fibrosis, in particular idiopathic pulmonary fibrosis. As indicated in the experimental part, in particular in Table 4, the compounds of formula (I) of the present invention show a notable potency with respect to their inhibitory activity on receptor ALK5, below about 10 nM, confirming that they are able to inhibit the ALK5 receptor involved in fibrosis and diseases that result from fibrosis. As indicated in the experimental part, comparative examples, in particular in Table 5, it is shown that, conversely to the compounds C1 characterized by lacking a pyrimidinyl, a pyridinyl or a pyridinyl condensed group linked to the amino group bearing the pyridazine ring, the presence of a pyrimidinyl, a pyridinyl or a pyridinyl condensed group linked to the amino group bearing the pyridazine ring in the present invention compounds unexpectedly and remarkably determines a relevant increase in the inhibitory activity on the ALK5 receptor. Advantageously, the compounds of the present invention are endowed with a very high potency, they could be administered in human at a lower dosage respect to the compounds of the prior art, thus reducing the adverse events that typically occur administering higher dosages of a drug. In addition to being notably potent with respect to their inhibitory activity on receptor ALK5, the compounds of the present invention are also characterized by a good inhalatory profile, that permits to act effectively on the lung compartment and have, at the same time, a low metabolic stability, that allows to minimize the drawbacks associated with the systemic exposure, such as safety and tolerability issues.

Therefore, the compounds of the present invention are particularly appreciated by the skilled person when looking at a suitable and efficacious compounds useful for the treatment of fibrosis, in particular idiopathic pulmonary fibrosis, administered by the inhalation route and characterized by a good inhalatory profile, that corresponds to a good activity on the lung, a good lung retention and to a low metabolic stability, that minimizes the systemic exposure and correlated safety issues.

Thus, in one aspect the present invention relates to a compound of general formula (I)

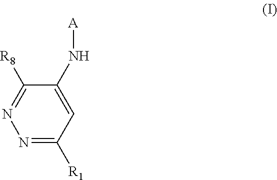

wherein
A is selected from the groups consisting of A1, A2, A3 and A4

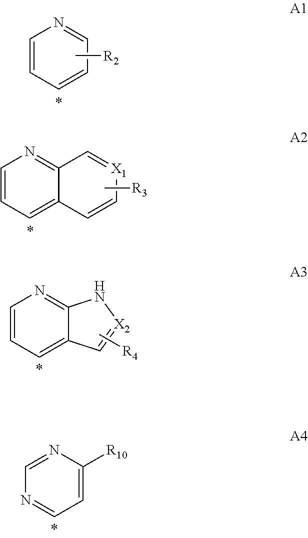

R₁ is selected from the group consisting of aryl and pyridyl, wherein said aryl and pyridyl are optionally substituted by one or more groups selected from halogen atoms and —(C₁-C₆)alkyl;
R₂ is selected from the group consisting of —NR₅C(O)R₆, —NR₅R₉ and —NH₂;
X₁ is C or CH;
X₂ is C, CH or N;
R₃ is —OR₇;
R₄ is H or —C(O)O—(C₁-C₆)alkyl;
R₅ is H or —(C₁-C₆)alkyl;
R₆ is selected from the group consisting of —(C₃-C₉)heterocycloalkyl substituted by one or more —(C₁-C₆)alkyl; —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl and —(C₃-C₆)cycloalkyl; —(C₁-C₆)alkylene-NH₂; —(C₃-C₆)cycloalkyl optionally substituted by one or more —(C₁₋C₆)alkylene-(C₃-C₉)heterocycloalkyl, wherein said —(C₃-C₉)heterocycloalkyl is optionally substituted by one or more groups selected from —(C₁-C₆)alkyl and —(C₃-C₆)cycloalkyl; and —(C₃-C₆)cycloalkyl optionally substituted by one or more —(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —O—(C₁-C₆)alkyl, —C(O)OH, —C(O)O—(C₁-C₆)alkyl, —(C₁-C₆)haloalkyl, —(C₃-C₆)cycloalkyl and halogen atoms;
R₇ is selected from the group consisting of —(C₁-C₆)alkyl and —(C₁-C₆)alkylene-(C₃-C₉)heterocycloalkyl, wherein said —(C₃-C₉)heterocycloalkyl is optionally substituted by one or more —(C₁-C₆)alkyl;
R₈ is selected from the group consisting of —NR_{A}R_{B}; —SH; —S—(C₁-C₆)alkyl, wherein said —(C₁-C₆)alkyl is optionally substituted by one or more —OH; —S—(C₁-C₆)alkylene-OH; —S—(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S(O)=NH—(C₁-C₆)alkyl; —S(O)₂—(C₁-C₆)alkyl; —S(O)—(C₁-C₆)alkyl; —S—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH and —OH; S—(C₁-C₆)alkylene-aryl, wherein said aryl is optionally substituted by one or more groups selected from —C(O)OH, —C(O)O—(C₁-C₆)alkylene-NR_{A}R_{C} and —C(O)O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-Si((C₁-C₆)alkyl)₃; —S—(C₁-C₆)alkylene-O—(C₁-C₆)alkylene-OH; —S—(C₁-C₆)alkylene-O—(C₁-C₆)alkylene-(C₃-C₉)heterocycloalkyl, wherein said —(C₃-C₉)heterocycloalkyl is optionally substituted by one or more groups selected from oxo and —(C₁-C₆)alkyl; —S—(C₁-C₆)alkylene-NH—C(O)—(C₃-C₉)heterocycloalkyl, wherein said —(C₃-C₉)heterocycloalkyl is optionally substituted by one or more oxo; —S—(C₁-C₆)alkylene-NH—(C₃-C₉)heterocycloalkyl, wherein said —(C₃-C₉)heterocycloalkyl is optionally substituted by one or more oxo; —O—(C₁-C₆)alkyl; —O—(C₁-C₆)haloalkyl; —O—(C₁-C₆)alkylene-OH, wherein said —O—(C₁-C₆)alkylene is substituted by one or more —OH; —O—(C₁-C₆)alkylene-C(O)O—(C₁-C₆)alkyl; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁-C₆)alkylene-S(O)—(C₁-C₆)alkyl; —O—(C₁-C₆)alkylene-S(O)₂—(C₁-C₆)alkyl; —O—(C₁-C₆)alkylene-NH—S(O)₂—(C₁-C₆)alkyl; —O—(C₁-C₆)alkylene-O—(C₁-C₆)alkyl; —O—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH, —C(O)O—(C₁-C₆)alkyl and —OH; —O—(C₃-C₆)cycloalkyl, wherein said —(C₃-C₆)cycloalkyl is optionally substituted by one or more groups selected from —(C₁-C₆)alkylene-OH and —OH; —O—(C₁-C₆)alkylene-aryl, wherein said aryl is optionally substituted by one or more —OH; —O—(C₁-C₆)alkylene-aryl, wherein said aryl is fused to a —(C₅-C₆)heterocycloalkyl, wherein said —(C₅-C₆)heterocycloalkyl is optionally substitued by one or more groups selected from oxo and —(C₁-C₆)alkyl; —O—(C₃-C₉)heterocycloalkyl; and —O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo;
R₉ is a heteroaryl optionally substituted by one or more groups selected from —C(O)O—(C₁-C₆)alkyl and —(C₃-C₉)heterocycloalkyl, wherein said —(C₃-C₉)heterocycloalkyl is optionally substituted by one or more —(C₁-C₆)alkyl;
R₁₀ is —NR₅C(O)R₆;
R_{A} is H or —(C₁-C₆)alkyl;
R_{B} is H or selected from the group consisting of —(C₁-C₆)alkyl optionally substituted by one or more groups selected from halogen and —OH; —S(O)₂—(C₁-C₆)alkyl; —(C₁-C₆)alkylene-aryl, wherein said aryl is susbtituted by —OH; —(C₁-C₆)alkylene-OH; —(C₃-C₉)heterocycloalkyl; —(C₁-C₆)alkylene-C(O)O—(C₁-C₆)alkyl; —(C₁-C₆)alkylene-aryl-OCO—(C₁-C₆)alkyl; and —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —(C₁-C₆)alkylene-O—(C₁-C₆)alkyl, —(C₁-C₆)haloalkyl and oxo; or alternatively R_{A} and R_{B} together with the nitrogen atom to which they are attached may form a —(C₃-C₆)heterocycloalkyl, wherein said —(C₃-C₆)heterocycloalkyl is optionally substituted by one or more groups selected from —C(O)OH, —(C₁-C₆)alkylene-OH, —C(O)O—(C₁-C₆)alkyl and oxo, or said —(C₃-C₆)heterocycloalkyl is optionally substituted on two adjacent carbon atoms forming an additional condensed —(C₅-C₆)heterocycloalkyl, optionally substituted by oxo;
R_{C} is —(C₁-C₆)alkyl; and pharmaceutically acceptable salts thereof.

In a more preferred embodiment the present invention refers to a compound of formula (I) wherein R₁ is phenyl substituted by fluorine and chlorine.

In another preferred embodiment the present invention refers to a compound of formula (I) wherein R₁ is pyridyl substituted by fluorine and methyl.

In another preferred embodiment the present invention refers to a compound of formula (I) wherein R₈ is selected from the group consisting of —NR_{A}R_{B}; —SH; —S—(C₁-C₆)alkyl, wherein said —(C₁-C₆)alkyl is optionally substituted by one or more —OH; —S—(C₁-C₆)alkylene-OH; —S—(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S(O)=NH—(C₁-C₆)alkyl; —S(O)₂—(C₁-C₆)alkyl; —S(O)—(C₁-C₆)alkyl; —S—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH and —OH; S—(C₁-C₆)alkylene-aryl, wherein said aryl is optionally substituted by one or more groups selected from —C(O)OH, —C(O)O—(C₁-C₆)alkylene-NR_{A}R_{C} and —C(O)O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-Si((C₁—C₆)alkyl)₃; —S—(C₁-C₆)alkylene-O—(C₁-C₆)alkylene-OH; —S—(C₁-C₆)alkylene-O—(C₁-C₆)alkylene-(C₃-C₉)heterocycloalkyl, wherein said —(C₃-C₉)heterocycloalkyl is optionally substituted by one or more groups selected from oxo and —(C₁-C₆)alkyl; —S—(C₁-C₆)alkylene-NH—C(O)—(C₃-C₉)heterocycloalkyl, wherein said —(C₃-C₉)heterocycloalkyl is optionally substituted by one or more oxo; —S—(C₁-C₆)alkylene-NH—(C₃-C₉)heterocycloalkyl, wherein said —(C₃-C₉)heterocycloalkyl is optionally substituted by one or more oxo; —O—(C₁-C₆)alkyl; —O—(C₁-C₆)haloalkyl; —O—(C₁-C₆)alkylene-OH, wherein said —O—(C₁-C₆)alkylene is substituted by one or more —OH; —O—(C₁-C₆)alkylene-C(O)O—(C₁-C₆)alkyl; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁-C₆)alkylene-S(O)—(C₁-C₆)alkyl; —O—(C₁-C₆)alkylene-S(O)₂—(C₁-C₆)alkyl; —O—(C₁-C₆)alkylene-NH—S(O)₂—(C₁-C₆)alkyl; —O—(C₁-C₆)alkylene-O—(C₁-C₆)alkyl; —O—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH, —C(O)O—(C₁-C₆)alkyl and —OH; —O—(C₃-C₆)cycloalkyl, wherein said —(C₃-C₆)cycloalkyl is optionally substituted by one or more groups selected from —(C₁-C₆)alkylene-OH and —OH; —O—(C₁-C₆)alkylene-aryl, wherein said aryl is optionally substituted by one or more —OH; —O—(C₁-C₆)alkylene-aryl, wherein said aryl is fused to a —(C₅-C₆)heterocycloalkyl, wherein said —(C₅-C₆)heterocycloalkyl is optionally substitued by one or more groups selected from oxo and —(C₁-C₆)alkyl; —O—(C₃-C₉)heterocycloalkyl; and —O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo.

In a particularly preferred embodiment the present invention refers to a compound of formula (I), wherein A is A1

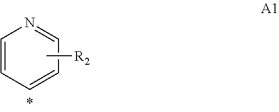

represented by the formula (Ia)

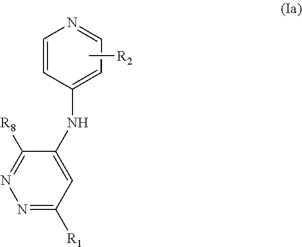

R₁ is selected from the group consisting of aryl and pyridyl, wherein said aryl and pyridyl are optionally substituted by one or more groups selected from —(C₁-C₆)alkyl and halogen atoms;
R₂ is selected from the group consisting of —NR₅C(O)R₆, —NR₅R₉ and —NH₂;
R₅ is H or —(C₁-C₆)alkyl;
R₆ is selected from the group consisting of —(C₃-C₉)heterocycloalkyl substituted by one or more —(C₁-C₆)alkyl; —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl and —(C₃-C₆)cycloalkyl; —(C₁-C₆)alkylene-NH₂; —(C₃-C₆)cycloalkyl optionally substituted by one or more —(C₁-C₆)alkylene-(C₃-C₉)heterocycloalkyl, wherein said —(C₃-C₉)heterocycloalkyl is optionally substituted by one or more groups selected from —(C₁-C₆)alkyl and —(C₃-C₆)cycloalkyl; and —(C₃-C₆)cycloalkyl optionally substituted by one or more —(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —O—(C₁-C₆)alkyl, —C(O)OH, —C(O)O—(C₁-C₆)alkyl, —(C₁-C₆)haloalkyl, —(C₃-C₆)cycloalkyl and halogen atoms;
R₈ is selected from the group consisting of —NR_{A}R_{B}; —SH; —S—(C₁-C₆)alkyl, wherein said —(C₁-C₆)alkyl is optionally substituted by one or more —OH; —S—(C₁-C₆)alkylene-OH; —S—(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S(O)=NH—(C₁-C₆)alkyl; —S(O)₂—(C₁-C₆)alkyl; —S(O)—(C₁-C₆)alkyl; —S—(C₁-C₆)alkylene-(C₃—C₆)cycloalkyl, wherein said —(C₃-C₆)cycloalkyl is optionally substituted by one or more groups selected from —(C₁-C₆)alkyl, —(C₁-C₆)alkylene-OH and —OH; S—(C₁-C₆)alkylene-aryl, wherein said aryl is optionally substituted by one or more groups selected from —C(O)OH, —C(O)O—(C₁-C₆)alkylene-NR_{A}R_{C} and —C(O)O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-Si((C₁-C₆)alkyl)₃; —S—(C₁-C₆)alkylene-O—(C₁-C₆)alkylene-OH; —S—(C₁-C₆)alkylene-O—(C₁-C₆)alkylene-(C₃-C₉)heterocycloalkyl, wherein said —(C₃-C₉)heterocycloalkyl is optionally substituted by one or more groups selected from oxo and —(C₁-C₆)alkyl; —S—(C₁-C₆)alkylene-NH—C(O)—(C₃-C₉)heterocycloalkyl, wherein said —(C₃-C₉)heterocycloalkyl is optionally substituted by one or more oxo; —S—(C₁-C₆)alkylene-NH—(C₃-C₉)heterocycloalkyl, wherein said —(C₃-C₉)heterocycloalkyl is optionally substituted by one or more oxo; —O—(C₁-C₆)alkyl; —O—(C₁-C₆)haloalkyl; —O—(C₁-C₆)alkylene-OH, wherein said —O—(C₁-C₆)alkylene is substituted by one or more —OH; —O—(C₁-C₆)alkylene-C(O)O—(C₁-C₆)alkyl; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁-C₆)alkylene-S(O)—(C₁-C₆)alkyl; —O—(C₁-C₆)alkylene-S(O)₂—(C₁-C₆)alkyl; —O—(C₁-C₆)alkylene-NH—S(O)₂—(C₁-C₆)alkyl; —O—(C₁-C₆)alkylene-O—(C₁-C₆)alkyl; —O—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH, —C(O)O—(C₁-C₆)alkyl and —OH; —O—(C₃-C₆)cycloalkyl, wherein said —(C₃-C₆)cycloalkyl is optionally substituted by one or more groups selected from —(C₁-C₆)alkylene-OH and —OH; —O—(C₁-C₆)alkylene-aryl, wherein said aryl is optionally substituted by one or more —OH; —O—(C₁-C₆)alkylene-aryl, wherein said aryl is fused to a —(C₅-C₆)heterocycloalkyl, wherein said —(C₅-C₆)heterocycloalkyl is optionally substitued by one or more groups selected from oxo and —(C₁-C₆)alkyl; —O—(C₃-C₉)heterocycloalkyl; and —O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo;
R₉ is a heteroaryl optionally substituted by one or more groups selected from —C(O)O—(C₁-C₆)alkyl and —(C₃-C₉)heterocycloalkyl, wherein said —(C₃-C₉)heterocycloalkyl is optionally substituted by one or more —(C₁-C₆)alkyl;
R_{A} is H or —(C₁₋C₆)alkyl;
R_{B} is H or is selected from the group consisting of —(C₁-C₆)alkyl optionally substituted by one or more groups selected from halogen and —OH; —S(O)₂—(C₁-C₆)alkyl; —(C₁-C₆)alkylene-aryl, wherein said aryl is susbtituted by —OH; —(C₃-C₉)heterocycloalkyl; —(C₁-C₆)alkylene-C(O)O—(C₁-C₆)alkyl; and —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —(C₁-C₆)alkylene-O—(C₁-C₆)alkyl and oxo; or alternatively R_{A} and R_{B} together with the nitrogen atom to which they are attached may form a —(C₃-C₆)heterocycloalkyl, wherein said —(C₃-C₆)heterocycloalkyl is optionally substituted by one or more groups selected from —C(O)OH, —(C₁-C₆)alkylene-OH, —C(O)O—(C₁-C₆)alkyl and oxo;
R_{C} is —(C₁-C₆)alkyl; and pharmaceutically acceptable salts thereof.

In another particularly preferred embodiment the present invention refers to a compound of formula (Ia), wherein R₁ is selected from the group consisting of aryl and pyridyl, wherein said aryl and pyridyl are optionally substituted by one or more groups selected from —(C₁-C₆)alkyl and halogen atoms;
R₂ is selected from the group consisting of —NR₅C(O)R₆ and —NH₂;
R₅ is H or —(C₁-C₆)alkyl;
R₆ is selected from the group consisting of heterocycloalkyl substituted by one or more —(C₁₋C₆)alkyl, and —(C₁₋C₆)alkylene-heterocycloalkyl, wherein said heterocycloalkyl is optionally substituted by one or more groups selected from —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl, -cycloalkyl and —(C₁-C₆)alkylene-NH₂;
R₈ is selected from the group consisting of —NR_{A}R_{B}; —S—(C₁-C₆)alkyl; —S—(C₁-C₆)alkylene-OH; —S(O)=NH—(C₁-C₆)alkyl; —S(O)₂—(C₁-C₆)alkyl; —S(O)—(C₁-C₆)alkyl; —O—(C₁-C₆)alkyl; —O—(C₁-C₆)haloalkyl; —O—(C₁-C₆)alkylene-OH; —O—(C₁-C₆)alkylene-C(O)O—(C₁-C₆)alkyl; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁₋C₆)alkylene-S(O)—(C₁-C₆)alkyl; —O—(C₁-C₆)alkylene-S(O)₂—(C₁-C₆)alkyl; —O—(C₁-C₆)alkylene-NH—S(O)₂—(C₁-C₆)alkyl; —O—(C₁-C₆)alkylene-O—(C₁-C₆)alkyl and —O—(C₁-C₆)alkylene-heterocycloalkyl, wherein said heterocycloalkyl is optionally substituted by one or more —(C₁-C₆)alkyl;
R_{A} is H or —(C₁₋C₆)alkyl;
R_{B} is H or selected from the group consisting of —(C₁-C₆)alkyl, —S(O)₂—(C₁-C₆)alkyl;
R_{C} is —(C₁-C₆)alkyl; and pharmaceutically acceptable salts thereof.

According to a preferred embodiment, the invention refers to at least one of the compounds of Formula (Ia) listed in the Table 1 below and pharmaceutically acceptable salts thereof. These compounds are particularly active on receptor ALK5, as shown in Table 4.

TABLE 1
List of preferred compounds of Formula (Ia)

| Example | | |
|---|---|---|
| No | Structure | Chemical name |
| 1 | 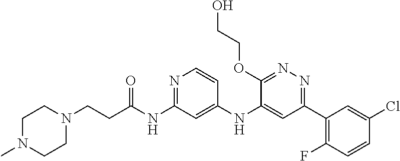 [SMILES: CN1CCN(CCC(=O)Nc2cc(Nc3cc(-c4cc(Cl)ccc4F)nnc3OCCO)ccn2)CC1] | N-(4-{[6-(5-chloro-2-fluoro- phenyl)-3-(2-hydroxyethoxy)- pyridazin-4-yl]amino}pyridin- 2-yl)-3-(4-methylpiperazin-1- yl)propanamide |
| 4 | 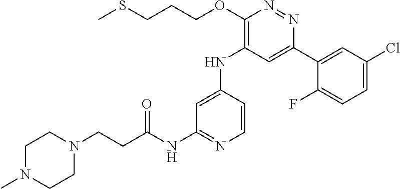 [SMILES: CSCCCOc1nnc(-c2cc(Cl)ccc2F)cc1Nc1ccnc(NC(=O)CCN2CCN(C)CC2)c1] | N-(4-{[6-(5-chloro-2-fluoro- phenyl)-3-[3-(methylsulfanyl)- propoxy]pyridazin-4-yl]- amino}pyridin-2-yl)-3-(4- methylpiperazin-1-yl)propan- amide |
| 7 | 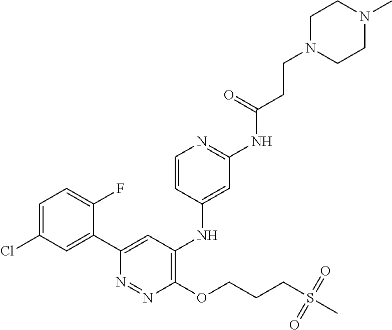 [SMILES: CN1CCN(CCC(=O)Nc2cc(Nc3cc(-c4cc(Cl)ccc4F)nnc3OCCCS(C)(=O)=O)ccn2)CC1] | N-(4-{[6-(5-chloro-2-fluoro- phenyl)-3-(3-methanesulfon- ylpropoxy)pyridazin-4-yl]- amino}pyridin-2-yl)-3-(4- methylpiperazin-1-yl)propan- amide |
| 8 | 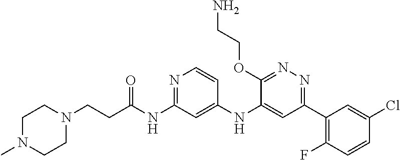 [SMILES: CN1CCN(CCC(=O)Nc2cc(Nc3cc(-c4cc(Cl)ccc4F)nnc3OCCN)ccn2)CC1] | N-(4-{[3-(2-aminoethoxy)-6- (5-chloro-2-fluorophenyl)- pyridazin-4-yl]amino}pyridin- 2-yl)-3-(4-methylpiperazin-1- yl)propanamide |
| 9 | 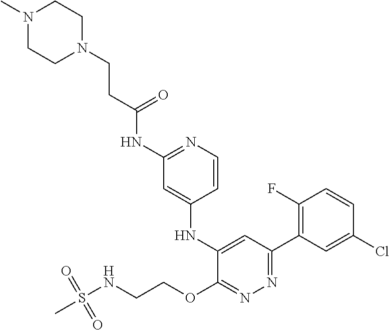 [SMILES: CN1CCN(CCC(=O)Nc2cc(Nc3cc(-c4cc(Cl)ccc4F)nnc3OCCNS(C)(=O)=O)ccn2)CC1] | N-(4-{[6-(5-chloro-2-fluoro- phenyl)-3-(2-methanesulfon- amidoethoxy)pyridazin-4-yl]- amino}pyridin-2-yl)-3-(4- methylpiperazin-1-yl)propan- amide |
| 12 | 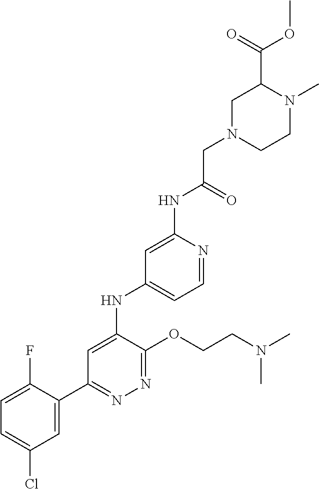 [SMILES: COC(=O)C1CN(CC(=O)Nc2cc(Nc3cc(-c4cc(Cl)ccc4F)nnc3OCCN(C)C)ccn2)CCN1C] | methyl 4-{[(4-{[6-(5-chloro- 2-fluorophenyl)-3-[2-(dimeth- ylamino)ethoxy]pyridazin-4- yl]amino}pyridin-2-yl)carbam- oyl]methyl}-1-methylpiper- azine-2-carboxylate |
| 13 | 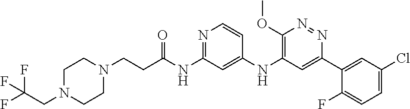 [SMILES: COc1nnc(-c2cc(Cl)ccc2F)cc1Nc1ccnc(NC(=O)CCN2CCN(CC(F)(F)F)CC2)c1] | N-(4-{[6-(5-chloro-2-fluoro- phenyl)-3-methoxypyridazin- 4-yl]amino}pyridin-2-yl)-3-[4- (2,2,2-trifluoroethyl)piperazin- 1-yl]propanamide |
| 14 | 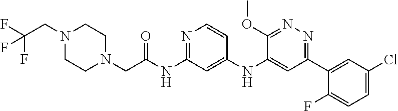 [SMILES: COc1nnc(-c2cc(Cl)ccc2F)cc1Nc1ccnc(NC(=O)CN2CCN(CC(F)(F)F)CC2)c1] | N-(4-{[6-(5-chloro-2-fluoro- phenyl)-3-methoxypyridazin- 4-yl]amino}pyridin-2-yl)-2-[4- (2,2,2-trifluoroethyl)piperazin- 1-yl]acetamide |
| 15 | 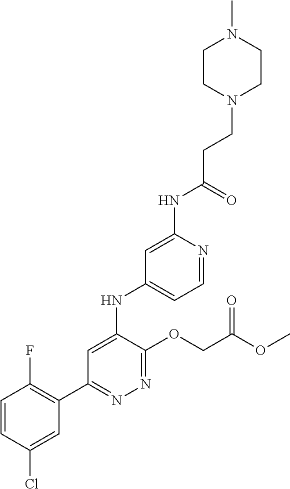 [SMILES: COC(=O)COc1nnc(-c2cc(Cl)ccc2F)cc1Nc1ccnc(NC(=O)CCN2CCN(C)CC2)c1] | methyl 2-{[6-(5-chloro-2- fluorophenyl)-4-({2-[3-(4- methylpiperazin-1-yl)propan- amido]pyridin-4-yl}amino)- pyridazin-3-yl]oxy}acetate |
| 16 | 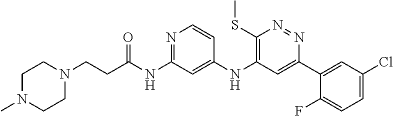 [SMILES: CSc1nnc(-c2cc(Cl)ccc2F)cc1Nc1ccnc(NC(=O)CCN2CCN(C)CC2)c1] | N-(4-{[6-(5-chloro-2-fluoro- phenyl)-3-(methylsulfanyl)- pyridazin-4-yl]amino}pyridin- 2-yl)-3-(4-methylpiperazin-1- yl)propanamide |
| 17 | 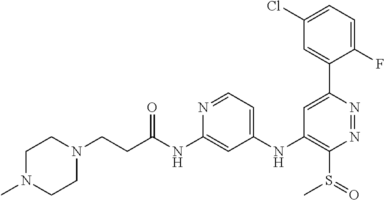 [SMILES: CN1CCN(CCC(=O)Nc2cc(Nc3cc(-c4cc(Cl)ccc4F)nnc3S(C)=O)ccn2)CC1] | N-(4-{[6-(5-chloro-2-fluoro- phenyl)-3-methanesulfinyl- pyridazin-4-yl]amino}pyridin- 2-yl)-3-(4-methylpiperazin-1- yl)propanamide |
| 18 | 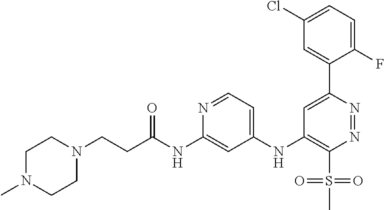 [SMILES: CN1CCN(CCC(=O)Nc2cc(Nc3cc(-c4cc(Cl)ccc4F)nnc3S(C)(=O)=O)ccn2)CC1] | N-(4-{[6-(5-chloro-2-fluoro- phenyl)-3-methanesulfonyl- pyridazin-4-yl]amino}pyridin- 2-yl)-3-(4-methylpiperazin-1- yl)propanamide |
| 19 | 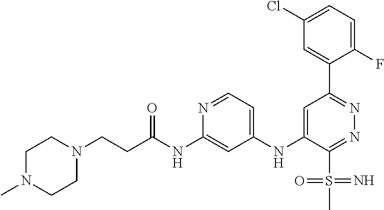 [SMILES: CN1CCN(CCC(=O)Nc2cc(Nc3cc(-c4cc(Cl)ccc4F)nnc3S(C)(=N)=O)ccn2)CC1] | N-(4-{[6-(5-chloro-2-fluoro- phenyl)-3-[imino(methyl)oxo- ??-sulfanyl]pyridazin-4-yl]- amino}pyridin-2-yl)-3-(4- methylpiperazin-1-yl)propan- amide |
| 20 | 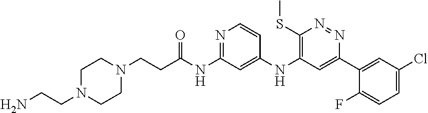 [SMILES: CSc1nnc(-c2cc(Cl)ccc2F)cc1Nc1ccnc(NC(=O)CCN2CCN(CCN)CC2)c1] | 3-[4-(2-aminoethyl)piperazin- 1-yl]-N-(4-{[6-(5-chloro-2- fluorophenyl)-3-(methylsulfan- yl)pyridazin-4-yl]amino}pyri- din-2-yl)propanamide |
| 21 | 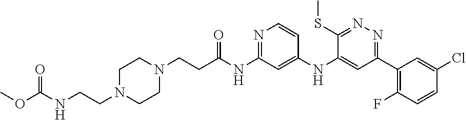 [SMILES: COC(=O)NCCN1CCN(CCC(=O)Nc2cc(Nc3cc(-c4cc(Cl)ccc4F)nnc3SC)ccn2)CC1] | methyl N-[2-(4-{2-[(4-{[6-(5- chloro-2-fluorophenyl)-3- (methylsulfanyl)pyridazin-4- yl]amino}pyridin-2-yl)- carbamoyl]ethyl}piperazin-1- yl)ethyl]carbamate |
| 22 | 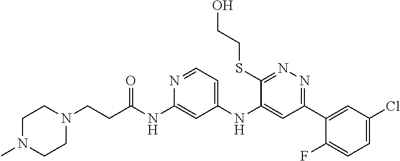 [SMILES: CN1CCN(CCC(=O)Nc2cc(Nc3cc(-c4cc(Cl)ccc4F)nnc3SCCO)ccn2)CC1] | N-(4-{[6-(5-chloro-2-fluoro- phenyl)-3-[(2-hydroxyethyl)- sulfanyl]pyridazin-4-yl]- amino}pyridin-2-yl)-3-(4- methylpiperazin-1-yl)propan- amide |
| 23 | 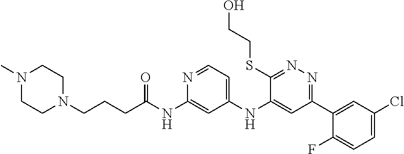 [SMILES: CN1CCN(CCCC(=O)Nc2cc(Nc3cc(-c4cc(Cl)ccc4F)nnc3SCCO)ccn2)CC1] | N-(4-{[6-(5-chloro-2-fluoro- phenyl)-3-[(2-hydroxyethyl)- sulfanyl]pyridazin-4-yl]- amino}pyridin-2-yl)-4-(4- methylpiperazin-1-yl)butan- amide |
| 24 | 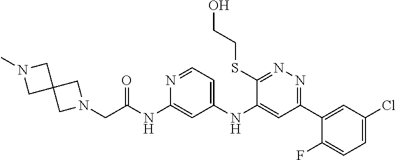 [SMILES: CN1CC2(C1)CN(CC(=O)Nc1cc(Nc3cc(-c4cc(Cl)ccc4F)nnc3SCCO)ccn1)C2] | N-(4-{[6-(5-chloro-2-fluoro- phenyl)-3-[(2-hydroxyethyl)- sulfanyl]pyridazin-4-yl]- amino}pyridin-2-yl)-2-{6- methyl-2,6-diazaspiro[3.3]- heptan-2-yl}acetamide |
| 25 | 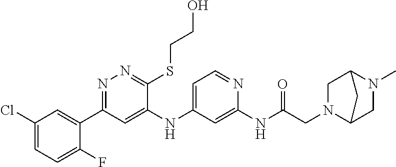 [SMILES: CN1CC2CC1CN2CC(=O)Nc1cc(Nc2cc(-c3cc(Cl)ccc3F)nnc2SCCO)ccn1] | N-(4-{[6-(5-chloro-2-fluoro- phenyl)-3-[(2-hydroxyethyl)- sulfanyl]pyridazin-4-yl]- amino}pyridin-2-yl)-2-{5- methyl-2,5-diazabicyclo- [2.2.1]heptan-2-yl}acetamide |
| 26 | 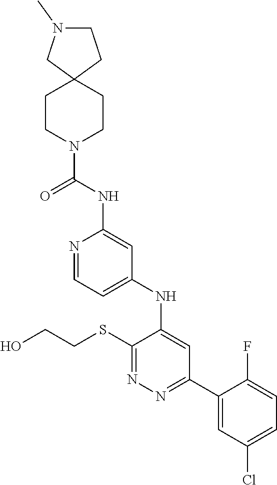 [SMILES: CN1CCC2(CCN(C(=O)Nc3cc(Nc4cc(-c5cc(Cl)ccc5F)nnc4SCCO)ccn3)CC2)C1] | N-(4-{[6-(5-chloro-2-fluoro- phenyl)-3-[(2-hydroxyethyl)- sulfanyl]pyridazin-4-yl]- amino}pyridin-2-yl)-2-methyl- 2,8-diazaspiro[4.5]decane-8- carboxamide |
| 27 | 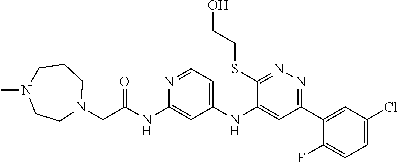 [SMILES: CN1CCCN(CC(=O)Nc2cc(Nc3cc(-c4cc(Cl)ccc4F)nnc3SCCO)ccn2)CC1] | N-(4-{[6-(5-chloro-2-fluoro- phenyl)-3-[(2-hydroxyethyl)- sulfanyl]pyridazin-4-yl]- amino}pyridin-2-yl)-2-(4- methyl-1,4-diazepan-1-yl)- acetamide |
| 28 | 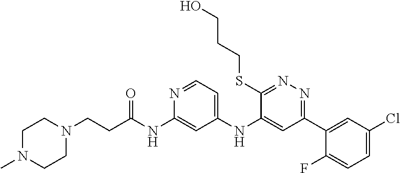 [SMILES: CN1CCN(CCC(=O)Nc2cc(Nc3cc(-c4cc(Cl)ccc4F)nnc3SCCCO)ccn2)CC1] | N-(4-{[6-(5-chloro-2-fluoro- phenyl)-3-[(3-hydroxyprop- yl)sulfanyl]pyridazin-4-yl]- amino}pyridin-2-yl)-3-(4- methylpiperazin-1-yl)propan- amide |
| 29 | 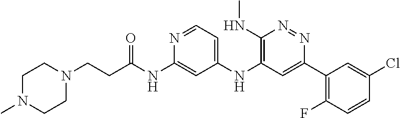 [SMILES: CNc1nnc(-c2cc(Cl)ccc2F)cc1Nc1ccnc(NC(=O)CCN2CCN(C)CC2)c1] | N-(4-{[6-(5-chloro-2-fluoro- phenyl)-3-(methylamino)- pyridazin-4-yl]amino}pyridin- 2-yl)-3-(4-methylpiperazin-1- yl)propanamide |
| 30 | 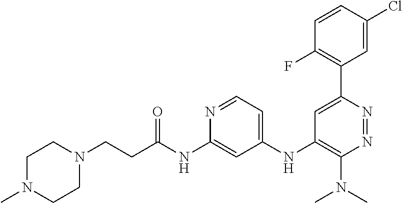 [SMILES: CN1CCN(CCC(=O)Nc2cc(Nc3cc(-c4cc(Cl)ccc4F)nnc3N(C)C)ccn2)CC1] | N-(4-{[6-(5-chloro-2-fluoro- phenyl)-3-(dimethylamino)- pyridazin-4-yl]amino}pyridin- 2-yl)-3-(4-methylpiperazin-1- yl)propanamide |
| 31 | 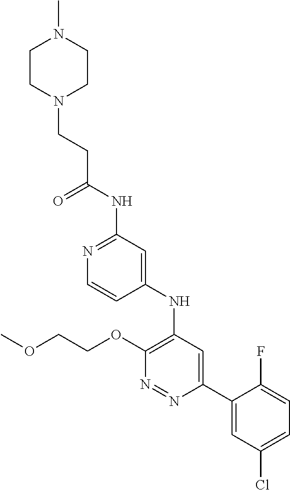 [SMILES: COCCOc1nnc(-c2cc(Cl)ccc2F)cc1Nc1ccnc(NC(=O)CCN2CCN(C)CC2)c1] | N-(4-{[6-(5-chloro-2-fluoro- phenyl)-3-(2-methoxyethoxy)- pyridazin-4-yl]amino}pyridin- 2-yl)-3-(4-methylpiperazin-1- yl)propanamide |
| 33 | 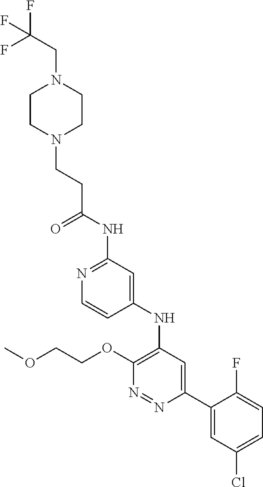 [SMILES: COCCOc1nnc(-c2cc(Cl)ccc2F)cc1Nc1ccnc(NC(=O)CCN2CCN(CC(F)(F)F)CC2)c1] | N-(4-{[6-(5-chloro-2-fluoro- phenyl)-3-(2-methoxyethoxy)- pyridazin-4-yl]amino}pyridin- 2-yl)-3-[4-(2,2,2-trifluoro- ethyl)piperazin-1-yl]propan- amide |
| 34 | 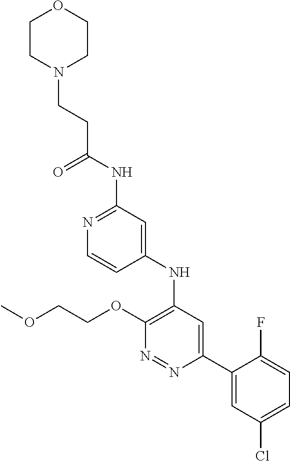 [SMILES: COCCOc1nnc(-c2cc(Cl)ccc2F)cc1Nc1ccnc(NC(=O)CCN2CCOCC2)c1] | N-(4-{[6-(5-chloro-2-fluoro- phenyl)-3-(2-methoxyethoxy)- pyridazin-4-yl]amino}pyridin- 2-yl)-3-(morpholin-4-yl)pro- panamide |
| 36 | 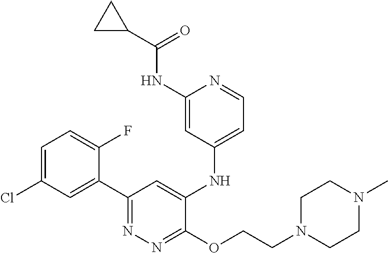 [SMILES: CN1CCN(CCOc2nnc(-c3cc(Cl)ccc3F)cc2Nc2ccnc(NC(=O)C3CC3)c2)CC1] | N-(4-{[6-(5-chloro-2-fluoro- phenyl)-3-[2-(4-methylpiper- azin-1-yl)ethoxy]pyridazin-4- yl]amino}pyridin-2-yl)cyclo- propanecarboxamide |
| 38 | 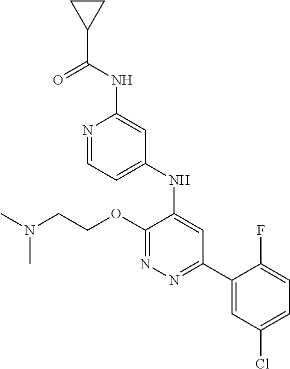 [SMILES: CN(C)CCOc1nnc(-c2cc(Cl)ccc2F)cc1Nc1ccnc(NC(=O)C2CC2)c1] | N-(4-{[6-(5-chloro-2-fluoro- phenyl)-3-[2-(dimethylamino)- ethoxy]pyridazin-4-yl]amino}- pyridin-2-yl)cyclopropane- carboxamide |
| 40 | 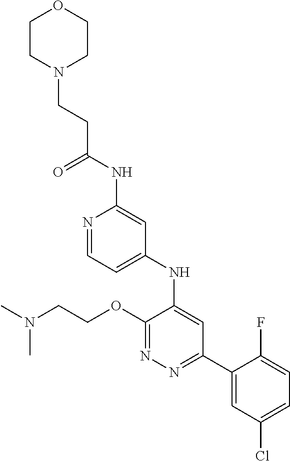 [SMILES: CN(C)CCOc1nnc(-c2cc(Cl)ccc2F)cc1Nc1ccnc(NC(=O)CCN2CCOCC2)c1] | N-(4-{[6-(5-chloro-2-fluoro- phenyl)-3-[2-(dimethylamino)- ethoxy]pyridazin-4-yl]amino}- pyridin-2-yl)-3-(morpholin-4- yl)propanamide |
| 43 | 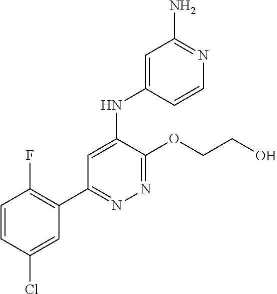 [SMILES: Nc1cc(Nc2cc(-c3cc(Cl)ccc3F)nnc2OCCO)ccn1] | 2-({4-[(2-aminopyridin-4-yl)- amino]-6-(5-chloro-2-fluoro- phenyl)pyridazin-3-yl}oxy)- ethan-1-ol |
| 44 | 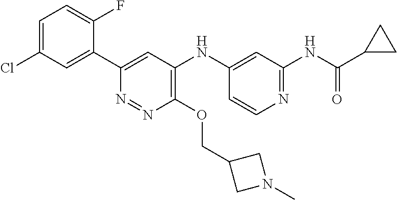 [SMILES: CN1CC(COc2nnc(-c3cc(Cl)ccc3F)cc2Nc2ccnc(NC(=O)C3CC3)c2)C1] | N-(4-{[6-(5-chloro-2-fluoro- phenyl)-3-[(1-methylazetidin- 3-yl)methoxy]pyridazin-4- yl]amino}pyridin-2-yl)cyclo- propanecarboxamide |
| 45 | 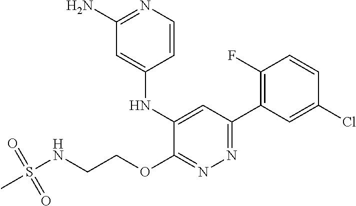 [SMILES: CS(=O)(=O)NCCOc1nnc(-c2cc(Cl)ccc2F)cc1Nc1ccnc(N)c1] | N-[2-({4-[(2-aminopyridin-4- yl)amino]-6-(5-chloro-2- fluorophenyl)pyridazin-3-yl}- oxy)ethyl]methanesulfonamide |
| 47 | 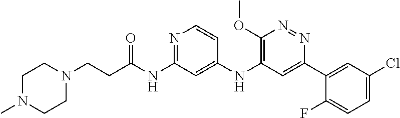 [SMILES: COc1nnc(-c2cc(Cl)ccc2F)cc1Nc1ccnc(NC(=O)CCN2CCN(C)CC2)c1] | N-(4-{[6-(5-chloro-2-fluoro- phenyl)-3-methoxypyridazin- 4-yl]amino}pyridin-2-yl)-3-(4- methylpiperazin-1-yl)propan- amide |
| 48 | 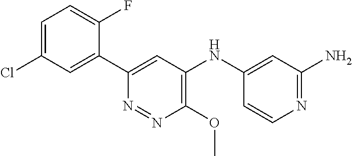 [SMILES: COc1nnc(-c2cc(Cl)ccc2F)cc1Nc1ccnc(N)c1] | N4-[6-(5-chloro-2-fluorophen- yl)-3-methoxypyridazin-4-yl]- pyridine-2,4-diamine |
| 49 | 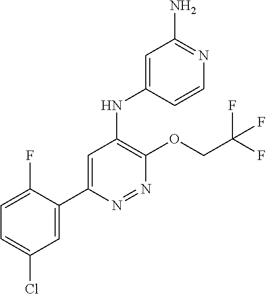 [SMILES: Nc1cc(Nc2cc(-c3cc(Cl)ccc3F)nnc2OCC(F)(F)F)ccn1] | N4-[6-(5-chloro-2-fluorophen- yl)-3-(2,2,2-trifluoroethoxy)- pyridazin-4-yl]pyridine-2,4- diamine |
| 50 | 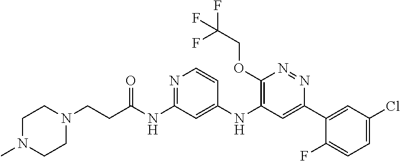 [SMILES: CN1CCN(CCC(=O)Nc2cc(Nc3cc(-c4cc(Cl)ccc4F)nnc3OCC(F)(F)F)ccn2)CC1] | N-(4-{[6-(5-chloro-2-fluoro- phenyl)-3-(2,2,2-trifluoro- ethoxy)pyridazin-4-yl]amino}- pyridin-2-yl)-3-(4-methylpiper- azin-1-yl)propanamide |
| 52 | 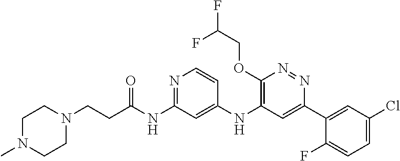 [SMILES: CN1CCN(CCC(=O)Nc2cc(Nc3cc(-c4cc(Cl)ccc4F)nnc3OCC(F)F)ccn2)CC1] | N-(4-{[6-(5-chloro-2-fluoro- phenyl)-3-(2,2-difluoroethoxy)- pyridazin-4-ylamino}pyridin-2- yl)-3-(4-methylpiperazin-1-yl)- propanamide |
| 53 | 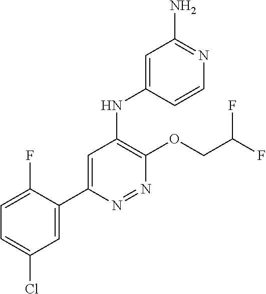 [SMILES: Nc1cc(Nc2cc(-c3cc(Cl)ccc3F)nnc2OCC(F)F)ccn1] | N4-[6-(5-chloro-2-fluorophen- yl)-3-(2,2-difluoroethoxy)- pyridazin-4-yl]pyridine-2,4- diamine |
| 54 | 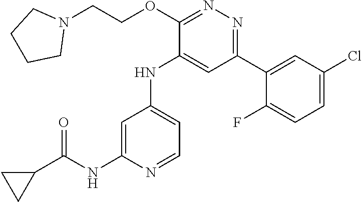 [SMILES: O=C(Nc1cc(Nc2cc(-c3cc(Cl)ccc3F)nnc2OCCN2CCCC2)ccn1)C1CC1] | N-(4-{[6-(5-chloro-2-fluoro- phenyl)-3-[2-(pyrrolidin-1-yl)- ethoxy]pyridazin-4-yl]amino}- pyridin-2-yl)cyclopropane- carboxamide |
| 55 | 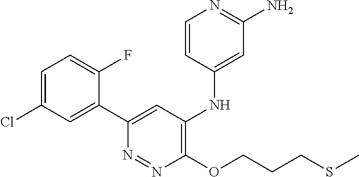 [SMILES: CSCCCOc1nnc(-c2cc(Cl)ccc2F)cc1Nc1ccnc(N)c1] | N4-[6-(5-chloro-2-fluorophen- yl)-3-[3-(methylsulfanyl)pro- poxy]pyridazin-4-yl]pyridine- 2,4-diamine |
| 56 | 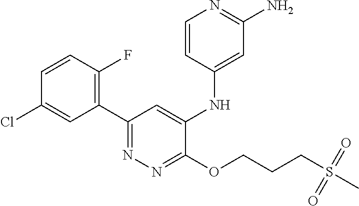 [SMILES: CS(=O)(=O)CCCOc1nnc(-c2cc(Cl)ccc2F)cc1Nc1ccnc(N)c1] | N4-[6-(5-chloro-2-fluorophen- yl)-3-(3-methanesulfonylpro- poxy)pyridazin-4-yl]pyridine- 2,4-diamine |
| 57 | 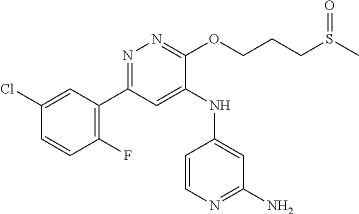 [SMILES: CS(=O)CCCOc1nnc(-c2cc(Cl)ccc2F)cc1Nc1ccnc(N)c1] | N4-[6-(5-chloro-2-fluorophen- yl)-3-(3-methanesulfinylpro- poxy)pyridazin-4-yl]pyridine- 2,4-diamine |
| 58 | 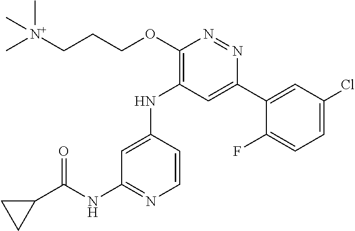 [SMILES: C[N+](C)(C)CCCOc1nnc(-c2cc(Cl)ccc2F)cc1Nc1ccnc(NC(=O)C2CC2)c1] | (3-{[6-(5-chloro-2-fluorophen- yl)-4-[(2-cyclopropaneamido- pyridin-4-yl)amino]pyridazin- 3-yl]oxy}propyl)trimethyl- azanium chloride |
| 60 | 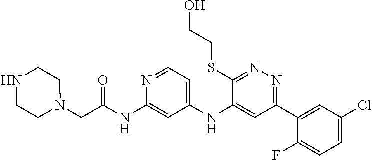 [SMILES: O=C(CN1CCNCC1)Nc1cc(Nc2cc(-c3cc(Cl)ccc3F)nnc2SCCO)ccn1] | N-(4-{[6-(5-chloro-2-fluoro- phenyl)-3-[(2-hydroxyethyl)- sulfanyl]pyridazin-4-yl]- amino}pyridin-2-yl)-2-(piper- azin-1-yl)acetamide |
| 61 | 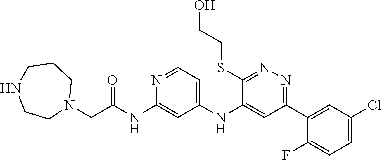 [SMILES: O=C(CN1CCCNCC1)Nc1cc(Nc2cc(-c3cc(Cl)ccc3F)nnc2SCCO)ccn1] | N-(4-{[6-(5-chloro-2-fluoro- phenyl)-3-[(2-hydroxyethyl)- sulfanyl]pyridazin-4-yl]- amino}pyridin-2-yl)-2-(1,4- diazepan-1-yl)acetamide |
| 62 | 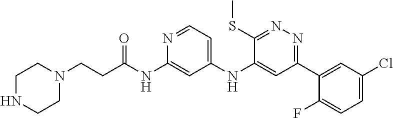 [SMILES: CSc1nnc(-c2cc(Cl)ccc2F)cc1Nc1ccnc(NC(=O)CCN2CCNCC2)c1] | N-(4-{[6-(5-chloro-2-fluoro- phenyl)-3-(methylsulfanyl)- pyridazin-4-yl]amino}pyridin- 2-yl)-3-(piperazin-1-yl)propan- amide |
| 68 | 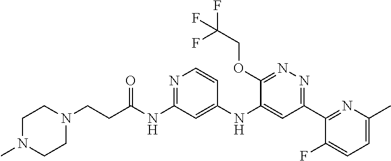 | N-(4-{[6-(3-fluoro-6-methyl- pyridin-2-yl)-3-(2,2,2-trifluoro- ethoxy)pyridazin-4-yl]amino}- pyridin-2-yl)-3-(4-methylpiper- azin-1-yl)propanamide |
| 69 | 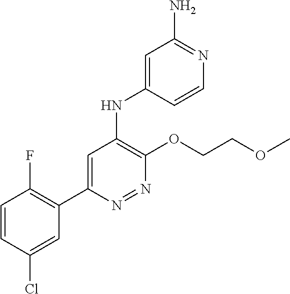 [SMILES: COCCOc1nnc(-c2cc(Cl)ccc2F)cc1Nc1ccnc(N)c1] | N4-[6-(5-chloro-2-fluorophen- yl)-3-(2-methoxyethoxy)pyri- dazin-4-yl]pyridine-2,4-diamine |
| 70 | 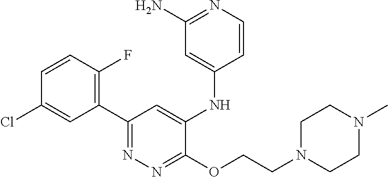 [SMILES: CN1CCN(CCOc2nnc(-c3cc(Cl)ccc3F)cc2Nc2ccnc(N)c2)CC1] | N4-[6-(5-chloro-2-fluorophen- yl)-3-[2-(4-methylpiperazin- 1-yl)ethoxy]pyridazin-4-yl]- pyridine-2,4-diamine |
| 71 | 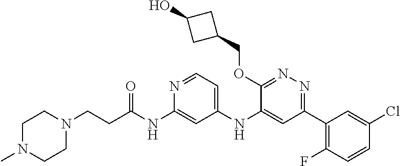 [SMILES: CN1CCN(CCC(=O)Nc2cc(Nc3cc(-c4cc(Cl)ccc4F)nnc3OC[C@H]3C[C@@H](O)C3)ccn2)CC1] | N-(4-{[6-(5-chloro-2-fluoro- phenyl)-3-{[(1s,3s)-3-hydroxy- cyclobutyl]methoxy}pyridazin- 4-yl]amino}pyridin-2-yl)-3-(4- methylpiperazin-1-yl)propan- amide |
| 72 | 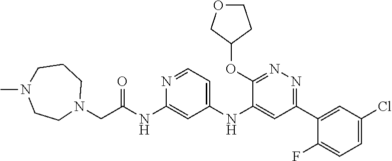 [SMILES: CN1CCCN(CC(=O)Nc2cc(Nc3cc(-c4cc(Cl)ccc4F)nnc3OC3CCOC3)ccn2)CC1] | N-(4-{[6-(5-chloro-2-fluoro- phenyl)-3-(oxolan-3-yloxy)- pyridazin-4-yl]amino}pyridin- 2-yl)-2-(4-methyl-1,4-diazepan- 1-yl)acetamide |
| 73 | 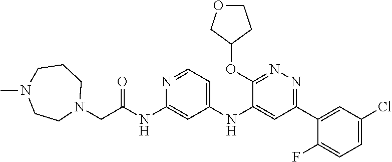 [SMILES: CN1CCCN(CC(=O)Nc2cc(Nc3cc(-c4cc(Cl)ccc4F)nnc3OC3CCOC3)ccn2)CC1] | Enantiomer 1 N-(4-{[6-(5- chloro-2-fluorophenyl)-3- (oxolan-3-yloxy)pyridazin-4- yl]amino}pyridin-2-yl)-2-(4- methyl-1,4-diazepan-1-yl)- acetamide |
| 74 | 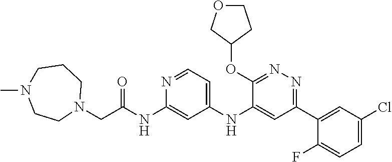 [SMILES: CN1CCCN(CC(=O)Nc2cc(Nc3cc(-c4cc(Cl)ccc4F)nnc3OC3CCOC3)ccn2)CC1] | Enantiomer 2 N-(4-{[6-(5- chloro-2-fluorophenyl)-3- (oxolan-3-yloxy)pyridazin-4- yl]amino}pyridin-2-yl)-2-(4- methyl-1,4-diazepan-1-yl)- acetamide |
| 75 | 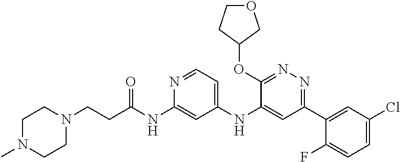 [SMILES: CN1CCN(CCC(=O)Nc2cc(Nc3cc(-c4cc(Cl)ccc4F)nnc3OC3CCOC3)ccn2)CC1] | Enantiomer 1 N-(4-{[6-(5- chloro-2-fluorophenyl)-3- (oxolan-3-yloxy)pyridazin-4- yl]amino}pyridin-2-yl)-3-(4- methylpiperazin-1-yl)propan- amide |
| 76 | 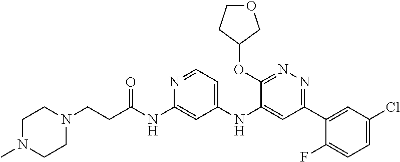 [SMILES: CN1CCN(CCC(=O)Nc2cc(Nc3cc(-c4cc(Cl)ccc4F)nnc3OC3CCOC3)ccn2)CC1] | Enantiomer 2 N-(4-{[6-(5- chloro-2-fluorophenyl)-3- (oxolan-3-yloxy)pyridazin-4- yl]amino}pyridin-2-yl)-3-(4- methylpiperazin-1-yl)propan- amide |
| 77 | 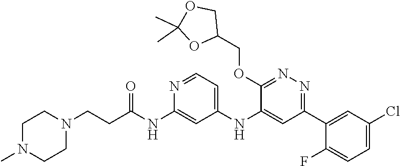 [SMILES: CN1CCN(CCC(=O)Nc2cc(Nc3cc(-c4cc(Cl)ccc4F)nnc3OCC3COC(C)(C)O3)ccn2)CC1] | N-(4-{[6-(5-chloro-2-fluoro- phenyl)-3-[(2,2-dimethyl-1,3- dioxolan-4-yl)methoxy]pyri- dazin-4-yl]amino}pyridin-2- yl)-3-(4-methylpiperazin-1-yl)- propanamide |
| 78 | 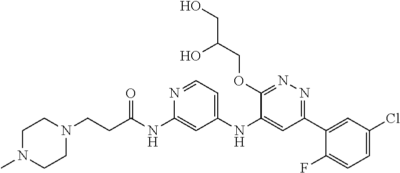 [SMILES: CN1CCN(CCC(=O)Nc2cc(Nc3cc(-c4cc(Cl)ccc4F)nnc3OCC(O)CO)ccn2)CC1] | N-(4-{[6-(5-chloro-2-fluoro- phenyl)-3-(2,3-dihydroxy- propoxy)pyridazin-4-yl]- amino}-pyridin-2-yl)-3-(4- methylpiperazin-1-yl)propan- amide |
| 79 | 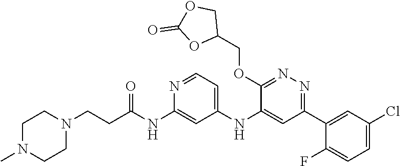 [SMILES: CN1CCN(CCC(=O)Nc2cc(Nc3cc(-c4cc(Cl)ccc4F)nnc3OCC3COC(=O)O3)ccn2)CC1] | N-(4-{[6-(5-chloro-2-fluoro- phenyl)-3-[(2-oxo-1,3-dioxo- lan-4-yl)methoxy]pyridazin-4- yl]amino}pyridin-2-yl)-3-(4- methylpiperazin-1-yl)propan- amide |
| 80 | 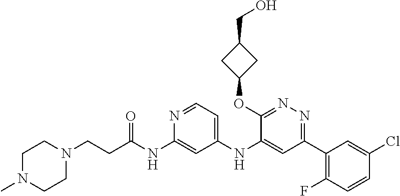 [SMILES: CN1CCN(CCC(=O)Nc2cc(Nc3cc(-c4cc(Cl)ccc4F)nnc3O[C@H]3C[C@@H](CO)C3)ccn2)CC1] | N-(4-{[6-(5-chloro-2-fluoro- phenyl)-3-[(1s,3s)-3-(hydroxy- methyl)cyclobutoxy]pyridazin- 4-yl]amino}pyridin-2-yl)-3-(4- methylpiperazin-1-yl)propan- amide |
| 81 | 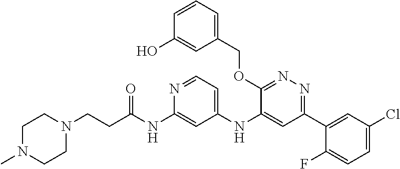 [SMILES: CN1CCN(CCC(=O)Nc2cc(Nc3cc(-c4cc(Cl)ccc4F)nnc3OCc3cccc(O)c3)ccn2)CC1] | N-(4-{[6-(5-chloro-2-fluoro- phenyl)-3-[(3-hydroxyphenyl)- methoxy]pyridazin-4-yl]- amino}pyridin-2-yl)-3-(4- methylpiperazin-1-yl)propan- amide |
| 82 | 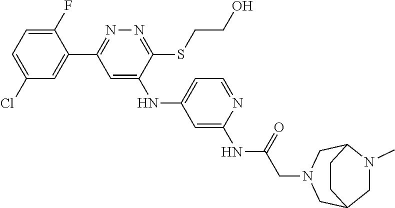 [SMILES: CN1CC2CCC1CN(CC(=O)Nc1cc(Nc3cc(-c4cc(Cl)ccc4F)nnc3SCCO)ccn1)C2] | N-(4-{[6-(5-chloro-2-fluoro- phenyl)-3-[(2-hydroxyethyl)- sulfanyl]pyridazin-4-yl]- amino}pyridin-2-yl)-2-{6- methyl-3,6-diazabicyclo[3.2.2]- nonan-3-yl}acetamide |
| 83 | 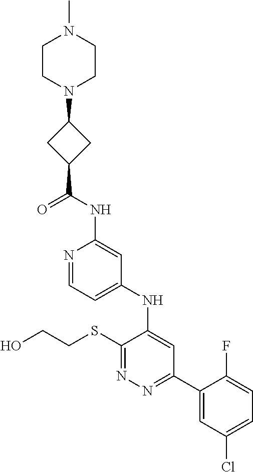 [SMILES: CN1CCN([C@H]2C[C@@H](C(=O)Nc3cc(Nc4cc(-c5cc(Cl)ccc5F)nnc4SCCO)ccn3)C2)CC1] | Cis-N-(4-{[6-(5-chloro-2- fluorophenyl)-3-[(2-hydroxy- ethyl)sulfanyl]pyridazin-4- yl]amino}pyridin-2-yl)-3-(4- methylpiperazin-1-yl)cyclo- butane-1-carboxamide |
| 84 | 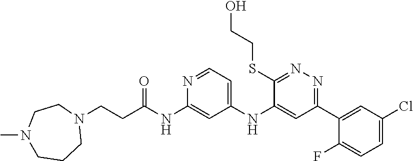 [SMILES: CN1CCCN(CCC(=O)Nc2cc(Nc3cc(-c4cc(Cl)ccc4F)nnc3SCCO)ccn2)CC1] | N-(4-{[6-(5-chloro-2-fluoro- phenyl)-3-[(2-hydroxyethyl)- sulfanyl]pyridazin-4-yl]amino}- pyridin-2-yl)-3-(4-methyl-1,4- diazepan-1-yl)propanamide |
| 85 | 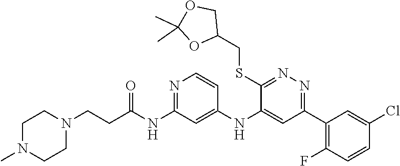 [SMILES: CN1CCN(CCC(=O)Nc2cc(Nc3cc(-c4cc(Cl)ccc4F)nnc3SCC3COC(C)(C)O3)ccn2)CC1] | N-(4-{[6-(5-chloro-2-fluoro- phenyl)-3-{[(2,2-dimethyl-1,3- dioxolan-4-yl)methyl]sulfanyl}- pyridazin-4-yl]amino}pyridin- 2-yl)-3-(4-methylpiperazin-1- yl)propanamide |
| 86 | 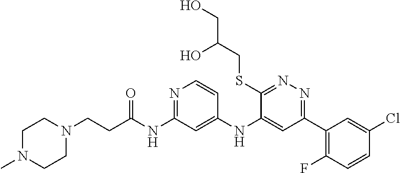 [SMILES: CN1CCN(CCC(=O)Nc2cc(Nc3cc(-c4cc(Cl)ccc4F)nnc3SCC(O)CO)ccn2)CC1] | N-(4-{[6-(5-chloro-2-fluoro- phenyl)-3-[(2,3-dihydroxy- propyl)sulfanyl]pyridazin-4- yl]amino}pyridin-2-yl)-3-(4- methylpiperazin-1-yl)propan- amide |
| 87 | 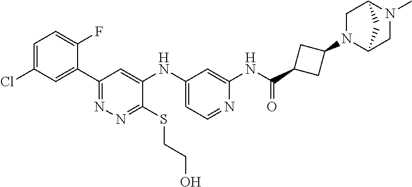 [SMILES: CN1C[C@@H]2C[C@H]1CN2[C@H]1C[C@@H](C(=O)Nc2cc(Nc3cc(-c4cc(Cl)ccc4F)nnc3SCCO)ccn2)C1] | Cis-N-(4-{[6-(5-chloro-2- fluorophenyl)-3-[(2-hydroxy- ethyl)sulfanyl]pyridazin-4-yl]- amino}pyridin-2-yl)-3- [(1S,4S)-5-methyl-2,5-diaza- bicyclo[2.2.1]heptan-2-yl]- cyclobutane-1-carboxamide |
| 88 | 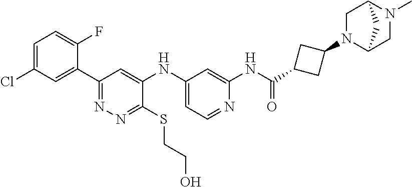 [SMILES: CN1C[C@@H]2C[C@H]1CN2[C@H]1C[C@H](C(=O)Nc2cc(Nc3cc(-c4cc(Cl)ccc4F)nnc3SCCO)ccn2)C1] | Trans-N-(4-{[6-(5-chloro-2- fluorophenyl)-3-[(2-hydroxy- ethyl)sulfanyl]pyridazin-4-yl]- amino}pyridin-2-yl)-3- [(1S,4S)-5-methyl-2,5-diaza- bicyclo[2.2.1]heptan-2-yl]- cyclobutane-1-carboxamide |
| 89 | 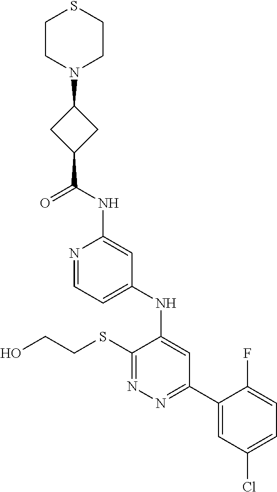 [SMILES: O=C(Nc1cc(Nc2cc(-c3cc(Cl)ccc3F)nnc2SCCO)ccn1)[C@H]1C[C@@H](N2CCSCC2)C1] | Cis N-(4-{[6-(5-chloro-2- fluorophenyl)-3-[(2-hydroxy- ethyl)sulfanyl]pyridazin-4-yl]- amino}pyridin-2-yl)-3-(thio- morpholin-4-yl)cyclobutane- 1-carboxamide |
| 90 | 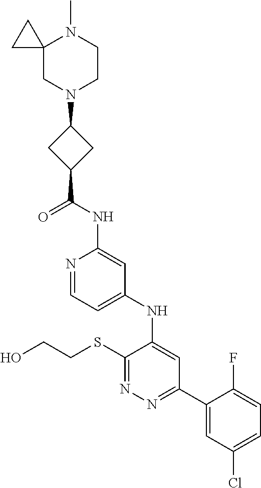 [SMILES: CN1CCN([C@H]2C[C@@H](C(=O)Nc3cc(Nc4cc(-c5cc(Cl)ccc5F)nnc4SCCO)ccn3)C2)CC12CC2] | Cis N-(4-{[6-(5-chloro-2- fluorophenyl)-3-[(2-hydroxy- ethyl)sulfanyl]pyridazin-4-yl]- amino}pyridin-2-yl)-3-{4- methyl-4,7-diazaspiro[2.5]- octan-7-yl}cyclobutane-1- carboxamide |
| 92 | 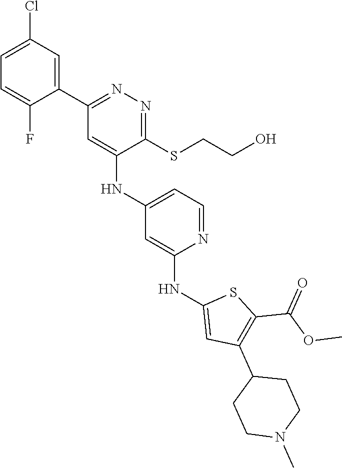 [SMILES: COC(=O)c1sc(Nc2cc(Nc3cc(-c4cc(Cl)ccc4F)nnc3SCCO)ccn2)cc1C1CCN(C)CC1] | methyl 5-[(4-{[6-(5-chloro-2- fluorophenyl)-3-[(2-hydroxy- ethyl)sulfanyl]pyridazin-4- yl]amino}pyridin-2-yl)amino]- 3-(1-methylpiperidin-4-yl)- thiophene-2-carboxylate |
| 93 | 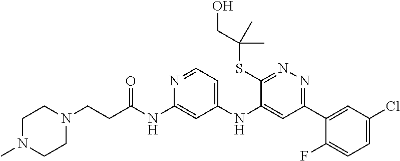 [SMILES: CN1CCN(CCC(=O)Nc2cc(Nc3cc(-c4cc(Cl)ccc4F)nnc3SC(C)(C)CO)ccn2)CC1] | N-(4-{[6-(5-chloro-2-fluoro- phenyl)-3-[(1-hydroxy-2- methylpropan-2-yl)sulfanyl]- pyridazin-4-yl]amino}pyridin- 2-yl)-3-(4-methylpiperazin-1- yl)propanamide |
| 94 | 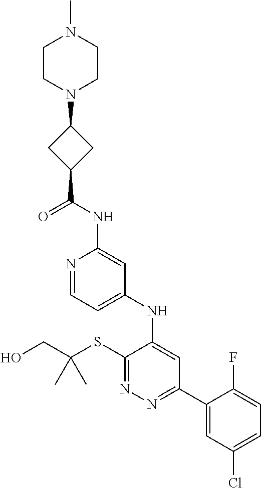 [SMILES: CN1CCN([C@H]2C[C@@H](C(=O)Nc3cc(Nc4cc(-c5cc(Cl)ccc5F)nnc4SC(C)(C)CO)ccn3)C2)CC1] | Cis N-(4-{[6-(5-chloro-2- fluorophenyl)-3-[(1-hydroxy- 2-methylpropan-2-yl)sulfan- yl]pyridazin-4-yl]amino}- pyridin-2-yl)-3-(4-methyl- piperazin-1-yl)cyclobutane- 1-carboxamide |
| 95 | 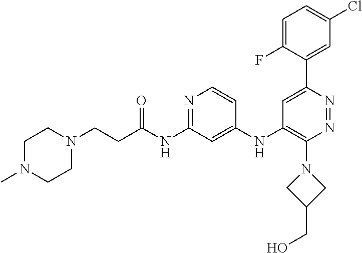 [SMILES: CN1CCN(CCC(=O)Nc2cc(Nc3cc(-c4cc(Cl)ccc4F)nnc3N3CC(CO)C3)ccn2)CC1] | N-(4-{[6-(5-chloro-2-fluoro- phenyl)-3-[3-(hydroxymeth- yl)azetidin-1-yl]pyridazin-4- yl]amino}pyridin-2-yl)-3-(4- methylpiperazin-1-yl)propan- amide |
| 96 | 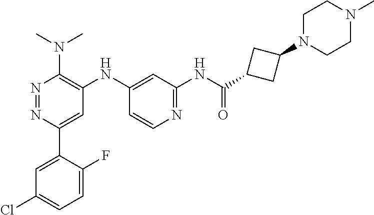 [SMILES: CN1CCN([C@H]2C[C@H](C(=O)Nc3cc(Nc4cc(-c5cc(Cl)ccc5F)nnc4N(C)C)ccn3)C2)CC1] | Trans-N-(4-{[6-(5-chloro-2- fluorophenyl)-3-(dimethyl- amino)pyridazin-4-yl]amino}- pyridin-2-yl)-3-(4-methyl- piperazin-1-yl)cyclobutane- 1-carboxamide |
| 97 | 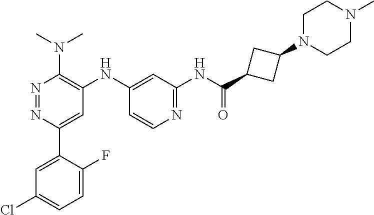 [SMILES: CN1CCN([C@H]2C[C@@H](C(=O)Nc3cc(Nc4cc(-c5cc(Cl)ccc5F)nnc4N(C)C)ccn3)C2)CC1] | Cis-N-(4-{[6-(5-chloro-2- fluorophenyl)-3-(dimethyl- amino)pyridazin-4-yl]amino}- pyridin-2-yl)-3-(4-methyl- piperazin-1-yl)cyclobutane- 1-carboxamide |
| 98 | 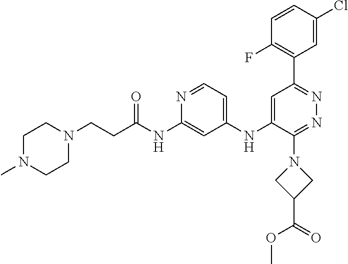 [SMILES: COC(=O)C1CN(c2nnc(-c3cc(Cl)ccc3F)cc2Nc2ccnc(NC(=O)CCN3CCN(C)CC3)c2)C1] | methyl 1-[6-(5-chloro-2- fluorophenyl)-4-({2-[3-(4- methylpiperazin-1-yl)propan- amido]pyridin-4-yl}amino)- pyridazin-3-yl]azetidine-3- carboxylate |
| 99 | 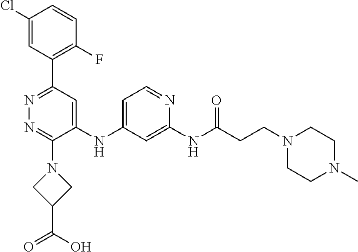 [SMILES: CN1CCN(CCC(=O)Nc2cc(Nc3cc(-c4cc(Cl)ccc4F)nnc3N3CC(C(=O)O)C3)ccn2)CC1] | 1-[6-(5-chloro-2-fluorophenyl)- 4-({2-[3-(4-methylpiperazin- 1-yl)propanamido]pyridin-4- yl}amino)pyridazin-3-yl]- azetidine-3-carboxylic acid |
| 100 | 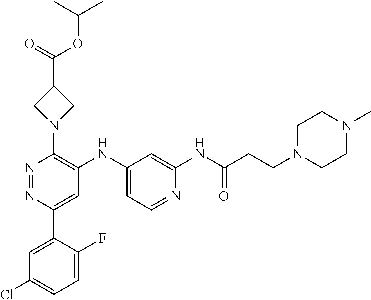 [SMILES: CC(C)OC(=O)C1CN(c2nnc(-c3cc(Cl)ccc3F)cc2Nc2ccnc(NC(=O)CCN3CCN(C)CC3)c2)C1] | propan-2-yl 1-[6-(5-chloro-2- fluorophenyl)-4-({2-[3-(4- methylpiperazin-1-yl)propan- amido]pyridin-4-yl}amino)- pyridazin-3-yl]azetidine-3- carboxylate |
| 101 | 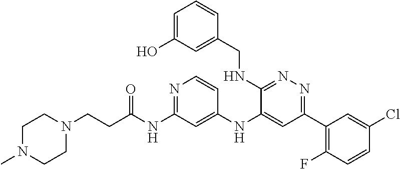 [SMILES: CN1CCN(CCC(=O)Nc2cc(Nc3cc(-c4cc(Cl)ccc4F)nnc3NCc3cccc(O)c3)ccn2)CC1] | N-(4-{[6-(5-chloro-2-fluoro- phenyl)-3-{[(3-hydroxyphen- yl)methyl]amino}pyridazin-4- yl]amino}pyridin-2-yl)-3-(4- methylpiperazin-1-yl)propan- amide |
| 102 | 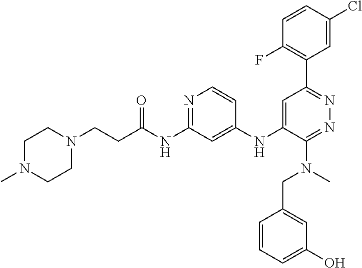 [SMILES: CN1CCN(CCC(=O)Nc2cc(Nc3cc(-c4cc(Cl)ccc4F)nnc3N(C)Cc3cccc(O)c3)ccn2)CC1] | N-(4-{[6-(5-chloro-2-fluoro- phenyl)-3-{[(3-hydroxyphen- yl)methyl](methyl)amino}- pyridazin-4-yl]amino}pyridin- 2-yl)-3-(4-methylpiperazin-1- yl)propanamide |
| 103 | 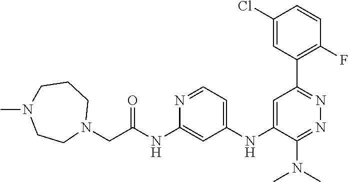 [SMILES: CN1CCCN(CC(=O)Nc2cc(Nc3cc(-c4cc(Cl)ccc4F)nnc3N(C)C)ccn2)CC1] | N-(4-{[6-(5-chloro-2-fluoro- phenyl)-3-(dimethylamino)- pyridazin-4-yl]amino}pyridin- 2-yl)-2-(4-methyl-1,4-di- azepan-1-yl)acetamide |
| 104 | 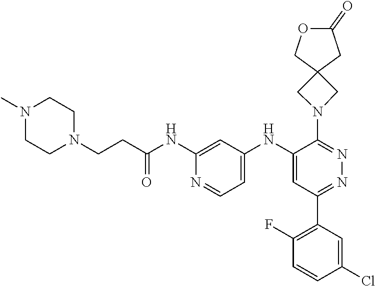 [SMILES: CN1CCN(CCC(=O)Nc2cc(Nc3cc(-c4cc(Cl)ccc4F)nnc3N3CC4(COC(=O)C4)C3)ccn2)CC1] | N-(4-{[6-(5-chloro-2-fluoro- phenyl)-3-{7-oxo-6-oxa-2- azaspiro[3.4]octan-2-yl}pyrid- azin-4-yl]amino}pyridin-2-yl)- 3-(4-methylpiperazin-1-yl)- propanamide |
| 105 | 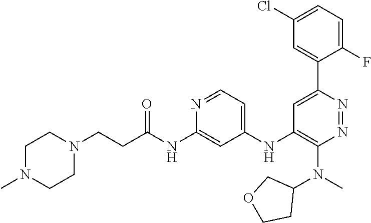 [SMILES: CN1CCN(CCC(=O)Nc2cc(Nc3cc(-c4cc(Cl)ccc4F)nnc3N(C)C3CCOC3)ccn2)CC1] | N-(4-{[6-(5-chloro-2-fluoro- phenyl)-3-[methyl(oxolan-3- yl)amino]pyridazin-4-yl]- amino}pyridin-2-yl)-3-(4- methylpiperazin-1-yl)propan- amide |
| 106 | 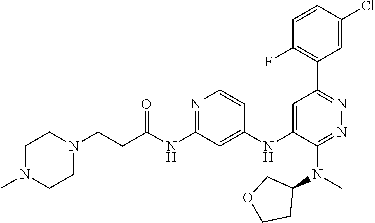 [SMILES: CN1CCN(CCC(=O)Nc2cc(Nc3cc(-c4cc(Cl)ccc4F)nnc3N(C)[C@H]3CCOC3)ccn2)CC1] | Enantiomer 1 N-(4-{[6-(5- chloro-2-fluorophenyl)-3- [methyl(oxolan-3-yl)amino]- pyridazin-4-yl]amino}pyridin- 2-yl)-3-(4-methylpiperazin- 1-yl)propanamide |
| 107 | 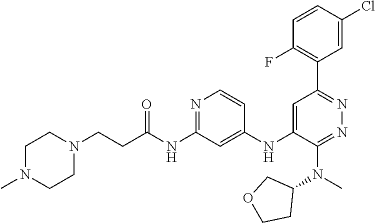 [SMILES: CN1CCN(CCC(=O)Nc2cc(Nc3cc(-c4cc(Cl)ccc4F)nnc3N(C)[C@@H]3CCOC3)ccn2)CC1] | Enantiomer 2 N-(4-{[6-(5- chloro-2-fluorophenyl)-3- [methyl(oxolan-3-yl)amino]- pyridazin-4-yl]amino}pyridin- 2-yl)-3-(4-methylpiperazin-1- yl)propanamide |
| 108 | 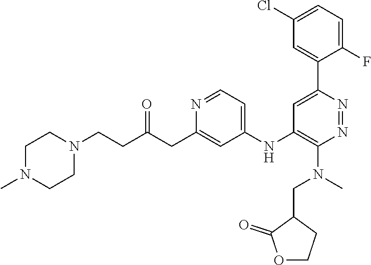 [SMILES: CN1CCN(CCC(=O)Cc2cc(Nc3cc(-c4cc(Cl)ccc4F)nnc3N(C)CC3CCOC3=O)ccn2)CC1] | N-(4-{[6-(5-chloro-2-fluoro- phenyl)-3-{methyl[(2-oxo- oxolan-3-yl)methyl]amino}- pyridazin-4-yl]amino}pyridin- 2-yl)-3-(4-methylpiperazin-1- yl)propanamide |
| 109 | 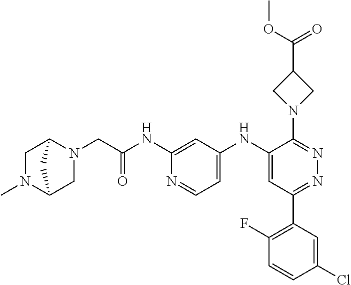 [SMILES: COC(=O)C1CN(c2nnc(-c3cc(Cl)ccc3F)cc2Nc2ccnc(NC(=O)CN3C[C@@H]4C[C@H]3CN4C)c2)C1] | methyl 1-[6-(5-chloro-2- fluorophenyl)-4-[(2-{2- [(1S,4S)-5-methyl-2,5-diaza- bicyclo[2.2.1]heptan-2-yl]- acetamido}pyridin-4-yl)- amino]pyridazin-3-yl]azeti- dine-3-carboxylate |
| 110 | 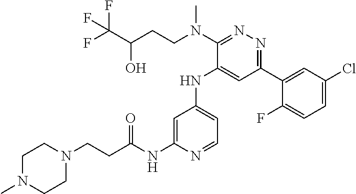 [SMILES: CN1CCN(CCC(=O)Nc2cc(Nc3cc(-c4cc(Cl)ccc4F)nnc3N(C)CCC(O)C(F)(F)F)ccn2)CC1] | N-(4-{[6-(5-chloro-2-fluoro- phenyl)-3-[methyl(4,4,4- trifluoro-3-hydroxybutyl)- amino]pyridazin-4-yl]amino}- pyridin-2-yl)-3-(4-methylpiper- azin-1-yl)propanamide |
| 111 | 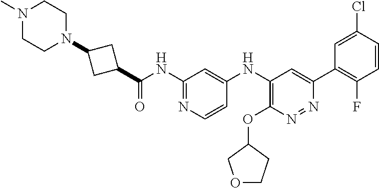 | Cis Enantiomer 1 N-(4-{[6- (5-chloro-2-fluorophenyl)-3- (oxolan-3-yloxy)pyridazin-4- yl]amino}pyridin-2-yl)-3-(4- methylpiperazin-1-yl)cyclo- butane-1-carboxamide |
| 112 | 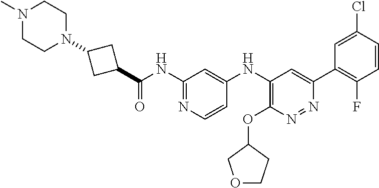 | Trans Enantiomer 1 N-(4-{[6- (5-chloro-2-fluorophenyl)-3- (oxolan-3-yloxy)pyridazin-4- ylamino}pyridin-2-yl)-3-(4- methylpiperazin-1-yl)cyclo- butane-1-carboxamide |
| 113 | 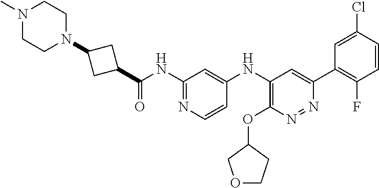 | Cis Enantiomer 2 N-(4-{[6- (5-chloro-2-fluorophenyl)-3- (oxolan-3-yloxy)pyridazin-4- yl]amino}pyridin-2-yl)-3-(4- methylpiperazin-1-yl)cyclo- butane-1-carboxamide |
| 114 | 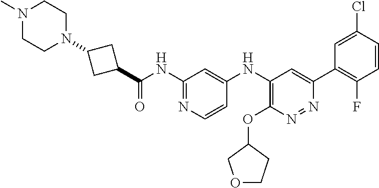 | Trans Enantiomer 2 N-(4-{[6- (5-chloro-2-fluorophenyl)-3- (oxolan-3-yloxy)pyridazin-4- yl]amino}pyridin-2-yl)-3-(4- methylpiperazin-1-yl)cyclo- butane-1-carboxamide |
| 115 | 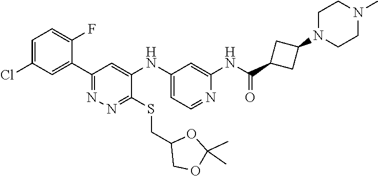 [SMILES: CN1CCN([C@H]2C[C@@H](C(=O)Nc3cc(Nc4cc(-c5cc(Cl)ccc5F)nnc4SCC4COC(C)(C)O4)ccn3)C2)CC1] | Cis N-(4-{[6-(5-chloro-2- fluorophenyl)-3-{[(2,2- dimethyl-1,3-dioxolan-4-yl)- methyl]sulfanyl}pyridazin-4- yl]amino}pyridin-2-yl)-3-(4- methylpiperazin-1-yl)cyclo- butane-1-carboxamide |
| 116 | 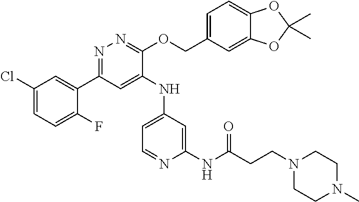 [SMILES: CN1CCN(CCC(=O)Nc2cc(Nc3cc(-c4cc(Cl)ccc4F)nnc3OCc3ccc4c(c3)OC(C)(C)O4)ccn2)CC1] | N-(4-{[6-(5-chloro-2-fluoro- phenyl)-3-[(2,2-dimethyl-2H- 1,3-benzodioxol-5-yl)meth- oxy]pyridazin-4-yl]amino}- pyridin-2-yl)-3-(4-methyl- piperazin-1-yl)propanamide |
| 117 | 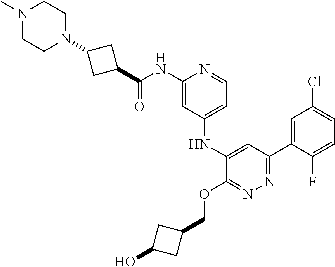 | Trans N-(4-{[6-(5-chloro-2- fluorophenyl)-3-[(3-hydroxy- cyclobutyl)methoxy]pyridazin- 4-yl]amino}pyridin-2-yl)-3-(4- methylpiperazin-1-yl)cyclo- butane-1-carboxamide |
| 118 | 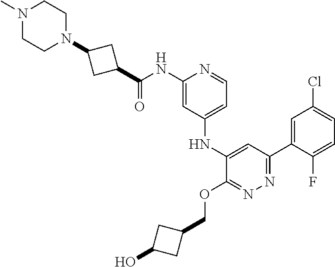 | Cis N-(4-{[6-(5-chloro-2- fluorophenyl)-3-[(3-hydroxy- cyclobutyl)methoxy]pyridazin- 4-yl]amino}pyridin-2-yl)-3-(4- methylpiperazin-1-yl)cyclo- butane-1-carboxamide |
| 119 | 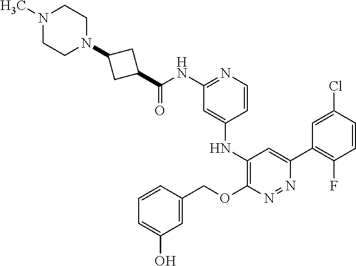 | Cis N-(4-{[6-(5-chloro-2- fluorophenyl)-3-[(3-hydroxy- phenyl)methoxy]pyridazin-4- yl]amino}pyridin-2-yl)-3-(4- methylpiperazin-1-yl)cyclo- butane-1-carboxamide |
| 120 | 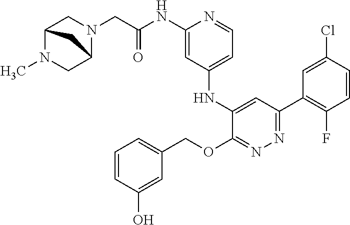 [SMILES: CN1C[C@@H]2C[C@H]1CN2CC(=O)Nc1cc(Nc2cc(-c3cc(Cl)ccc3F)nnc2OCc2cccc(O)c2)ccn1] | N-(4-{[6-(5-chloro-2-fluoro- phenyl)-3-[(3-hydroxyphenyl)- methoxy]pyridazin-4-yl]- amino}pyridin-2-yl)-2- [(1S,4S)-5-methyl-2,5-diaza- bicyclo[2.2.1]heptan-2-yl]acet- amide |
| 121 | 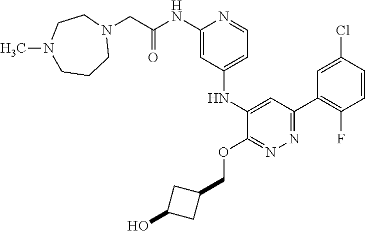 | N-(4-{[6-(5-chloro-2-fluoro- phenyl)-3-[(3-hydroxycyclo- butyl)methoxy]pyridazin-4- yl]amino}pyridin-2-yl)-2-(4- methyl-1,4-diazepan-1-yl)- acetamide |
| 122 | 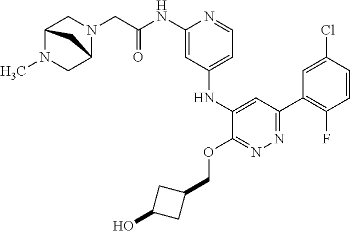 | N-(4-{[6-(5-chloro-2-fluoro- phenyl)-3-[(3-hydroxycyclo- butyl)methoxy]pyridazin-4- yl]amino}pyridin-2-yl)-2- [(1S,4S)-5-methyl-2,5-diaza- bicyclo[2.2.1]heptan-2-yl]- acetamide |
| 123 | 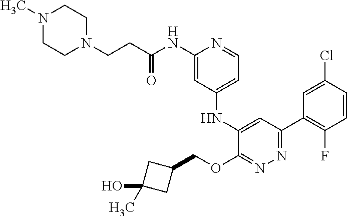 | N-(4-{[6-(5-chloro-2-fluoro- phenyl)-3-[(3-hydroxy-3- methylcyclobutyl)methoxyl- pyridazin-4-yl]amino}pyridin- 2-yl)-3-(4-methylpiperazin-1- yl)propanamide |
| 124 | 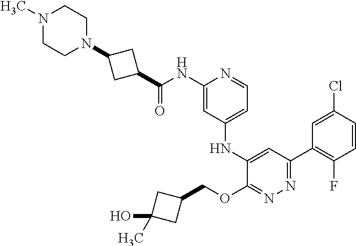 | Cis N-(4-{[6-(5-chloro-2- fluorophenyl)-3-[(3-hydroxy- 3-methylcyclobutyl)methoxy]- pyridazin-4-yl]amino}pyridin- 2-yl)-3-(4-methylpiperazin-1- yl)cyclobutane-1-carboxamide |
| 125 | 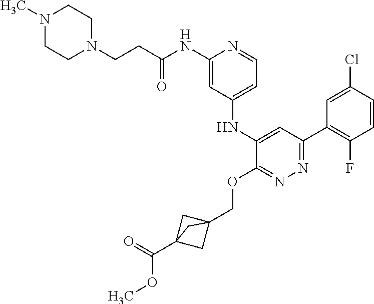 [SMILES: COC(=O)C12CC(COc3nnc(-c4cc(Cl)ccc4F)cc3Nc3ccnc(NC(=O)CCN4CCN(C)CC4)c3)(C1)C2] | methyl 3-({[6-(5-chloro-2- fluorophenyl)-4-({2-[3-(4- methylpiperazin-1-yl)propan- amido]pyridin-4-yl}amino)- pyridazin-3-yl]oxy}methyl)- bicyclo[1.1.1]pentane-1- carboxylate |
| 126 | 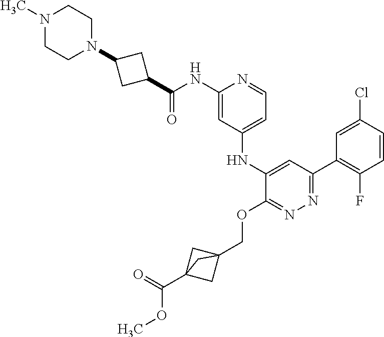 | Cis methyl 3-({[6-(5-chloro- 2-fluorophenyl)-4-({2-[-3-(4- methylpiperazin-1-yl)cyclo- butaneamido]pyridin-4-yl}- amino)pyridazin-3-yl]oxy}- methyl)bicyclo[1.1.1]pentane- 1-carboxylate |
| 127 | 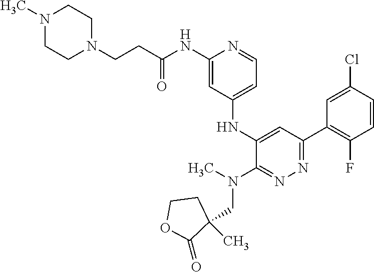 [SMILES: CN1CCN(CCC(=O)Nc2cc(Nc3cc(-c4cc(Cl)ccc4F)nnc3N(C)C[C@@]3(C)CCOC3=O)ccn2)CC1] | N-(4-{[6-(5-chloro-2-fluoro- phenyl)-3-{methyl[(3-methyl- 2-oxooxolan-3-yl)methyl]- amino}pyridazin-4-yl]amino}- pyridin-2-yl)-3-(4-methylpiper- azin-1-yl)propanamide |
| 128 | 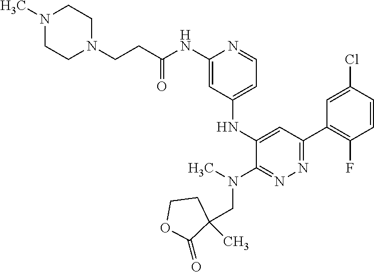 [SMILES: CN1CCN(CCC(=O)Nc2cc(Nc3cc(-c4cc(Cl)ccc4F)nnc3N(C)CC3(C)CCOC3=O)ccn2)CC1] | Enantiomer 1 N-(4-{[6-(5- chloro-2-fluorophenyl)-3- {methyl[(3-methyl-2-oxo- oxolan-3-yl)methyl]amino}- pyridazin-4-yl]amino}pyridin- 2-yl)-3-(4-methylpiperazin-1- yl)propanamide |
| 129 | 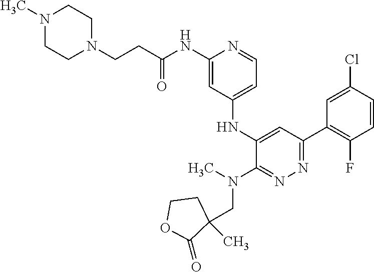 [SMILES: CN1CCN(CCC(=O)Nc2cc(Nc3cc(-c4cc(Cl)ccc4F)nnc3N(C)CC3(C)CCOC3=O)ccn2)CC1] | Enantiomer 2 N-(4-{[6-(5- chloro-2-fluorophenyl)-3- {methyl[(3-methyl-2-oxo- oxolan-3-yl)methyl]amino}- pyridazin-4-yl]amino}pyridin- 2-yl)-3-(4-methylpiperazin-1- yl)propanamide |
| 130 | 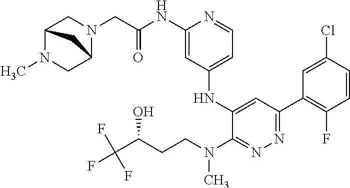 [SMILES: CN(CC[C@@H](O)C(F)(F)F)c1nnc(-c2cc(Cl)ccc2F)cc1Nc1ccnc(NC(=O)CN2C[C@@H]3C[C@H]2CN3C)c1] | N-(4-{[6-(5-chloro-2-fluoro- phenyl)-3-[methyl(4,4,4- trifluoro-3-hydroxybutyl)- amino]pyridazin-4-yl]amino}- pyridin-2-yl)-2-[(1S,4S)-5- methyl-2,5-diazabicyclo- [2.2.1]heptan-2-yl]acetamide |
| 131 | 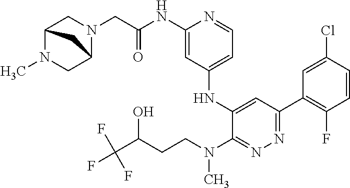 [SMILES: CN(CCC(O)C(F)(F)F)c1nnc(-c2cc(Cl)ccc2F)cc1Nc1ccnc(NC(=O)CN2C[C@@H]3C[C@H]2CN3C)c1] | Diastereomer 1 N-(4-{[6-(5- chloro-2-fluorophenyl)-3- [methyl(4,4,4-trifluoro-3- hydroxybutyl)amino]pyridazin- 4-yl]amino}pyridin-2-yl)-2- [(1S,4S)-5-methyl-2,5-diaza- bicyclo[2.2.1]heptan-2-yl]- acetamide |
| 132 | 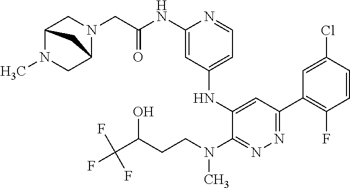 [SMILES: CN(CCC(O)C(F)(F)F)c1nnc(-c2cc(Cl)ccc2F)cc1Nc1ccnc(NC(=O)CN2C[C@@H]3C[C@H]2CN3C)c1] | Diastereomer 2 N-(4-{[6-(5- chloro-2-fluorophenyl)-3- [methyl(4,4,4-trifluoro-3- hydroxybutyl)amino]pyridazin- 4-yl]amino}pyridin-2-yl)-2- [(1S,4S)-5-methyl-2,5-diaza- bicyclo[2.2.1]heptan-2- yl]acetamide |
| 133 | 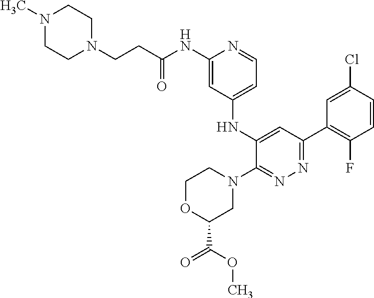 [SMILES: COC(=O)[C@H]1CN(c2nnc(-c3cc(Cl)ccc3F)cc2Nc2ccnc(NC(=O)CCN3CCN(C)CC3)c2)CCO1] | methyl 4-[6-(5-chloro-2- fluorophenyl)-4-({2-[3-(4- methylpiperazin-1-yl)propan- amido]pyridin-4-yl}amino)- pyridazin-3-yl]morpholine-2- carboxylate |
| 134 | 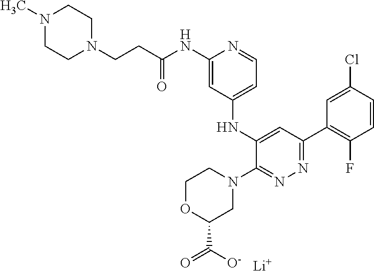 [SMILES: CN1CCN(CCC(=O)Nc2cc(Nc3cc(-c4cc(Cl)ccc4F)nnc3N3CCO[C@@H](C(=O)[O-])C3)ccn2)CC1 ; [Li+]] | 4-[6-(5-chloro-2-fluorophenyl)- 4-({2-[3-(4-methylpiperazin-1- yl)propanamido]pyridin-4- yl}amino)pyridazin-3-yl]- morpholine-2-carboxylate lithium salt |
| 135 | 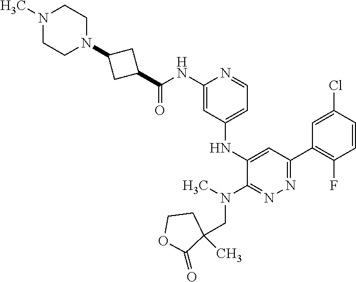 | Cis Enantiomer 1 N-(4-{[6- (5-chloro-2-fluorophenyl)-3- {methyl[(3-methyl-2-oxo- oxolan-3-yl)methyl]amino}- pyridazin-4-yl]amino}pyridin- 2-yl)-3-(4-methylpiperazin-1- yl)cyclobutane-1-carboxamide |
| 136 | 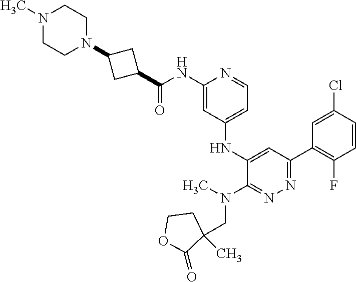 | Cis Enantiomer 2 N-(4-{[6- (5-chloro-2-fluorophenyl)-3- {methyl[(3-methyl-2-oxo- oxolan-3-yl)methyl]amino}- pyridazin-4-yl]amino}pyridin- 2-yl)-3-(4-methylpiperazin-1- yl)cyclobutane-1-carboxamide |
| 140 | 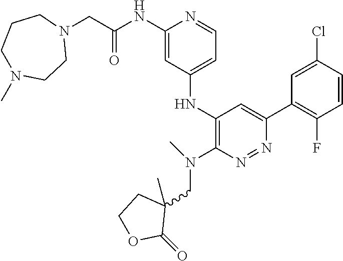 [SMILES: CN1CCCN(CC(=O)Nc2cc(Nc3cc(-c4cc(Cl)ccc4F)nnc3N(C)CC3(C)CCOC3=O)ccn2)CC1] | N-(4-((6-(5-chloro-2-fluoro- phenyl)-3-(methyl((3-methyl- 2-oxooxolan-3-yl)methyl)- amino)pyridazin-4-yl)amino)- pyridin-2-yl)-2-(4-methyl-1,4- diazepan-1-yl)acetamide |
| 141 | 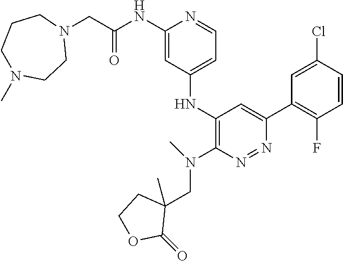 [SMILES: CN1CCCN(CC(=O)Nc2cc(Nc3cc(-c4cc(Cl)ccc4F)nnc3N(C)CC3(C)CCOC3=O)ccn2)CC1] | Enantiomer 1 N-(4-((6-(5- chloro-2-fluorophenyl)-3- (methyl((3-methyl-2-oxo- oxolan-3-yl)methyl)amino)- pyridazin-4-yl)amino)pyridin- 2-yl)-2-(4-methyl-1,4- diazepan-1-yl)acetamide |
| 142 | 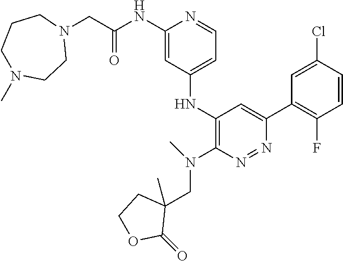 [SMILES: CN1CCCN(CC(=O)Nc2cc(Nc3cc(-c4cc(Cl)ccc4F)nnc3N(C)CC3(C)CCOC3=O)ccn2)CC1] | Enantiomer 2 N-(4-((6-(5- chloro-2-fluorophenyl)-3- (methyl((3-methyl-2-oxo- oxolan-3-yl)methyl)amino)- pyridazin-4-yl)amino)pyridin- 2-yl)-2-(4-methyl-1,4- diazepan-1-yl)acetamide |
| 145 | 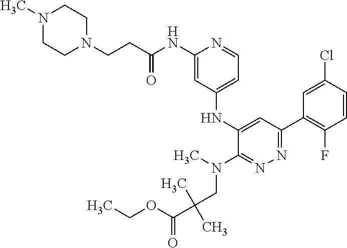 [SMILES: CCOC(=O)C(C)(C)CN(C)c1nnc(-c2cc(Cl)ccc2F)cc1Nc1ccnc(NC(=O)CCN2CCN(C)CC2)c1] | ethyl 3-{[6-(5-chloro-2- fluorophenyl)-4-({2-[3-(4- methylpiperazin-1-yl)- propanamido]pyridin-4-yl}- amino)pyridazin-3-yl](meth- yl)amino}-2,2-dimethyl- propanoate |
| 146 | 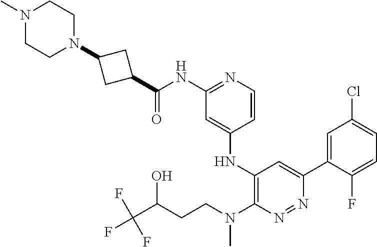 | Cis Enantiomer 1 N-(4-{[6- (5-chloro-2-fluorophenyl)-3- [methyl(4,4,4-trifluoro-3- hydroxybutyl)amino]pyridazin- 4-yl]amino}pyridin-2-yl)-3-(4- methylpiperazin-1-yl)cyclo- butane-1-carboxamide |
| 147 | 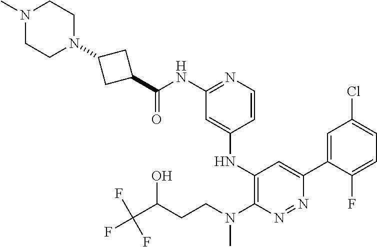 | Trans Enantiomer 1 N-(4-{[6- (5-chloro-2-fluorophenyl)-3- [methyl(4,4,4-trifluoro-3- hydroxybutyl)amino]pyridazin- 4-yl]amino}pyridin-2-yl)-3-(4- methylpiperazin-1-yl)cyclo- butane-1-carboxamide |
| 148 | 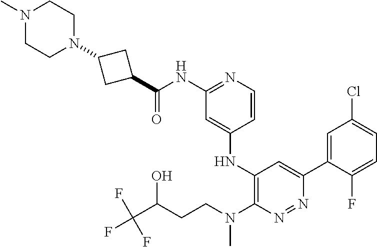 | Trans Enantiomer 2 N-(4-{[6- (5-chloro-2-fluorophenyl)-3- [methyl(4,4,4-trifluoro-3- hydroxybutyl)amino]pyridazin- 4-yl]amino}pyridin-2-yl)-3-(4- methylpiperazin-1-yl)cyclo- butane-1-carboxamide |
| 149 | 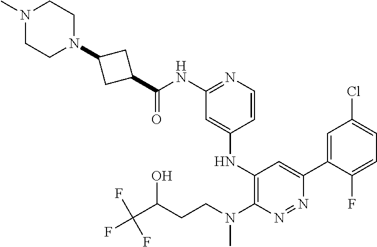 | Cis Enantiomer 2 N-(4-{[6- (5-chloro-2-fluorophenyl)-3- [methyl(4,4,4-trifluoro-3- hydroxybutyl)amino]pyridazin- 4-yl]amino}pyridin-2-yl)-3-(4- methylpiperazin-1-yl)cyclo- butane-1-carboxamide |
| 150 | 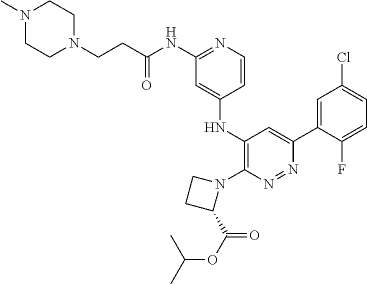 [SMILES: CC(C)OC(=O)[C@@H]1CCN1c1nnc(-c2cc(Cl)ccc2F)cc1Nc1ccnc(NC(=O)CCN2CCN(C)CC2)c1] | propan-2-yl 1-[6-(5-chloro-2- fluorophenyl)-4-({2-[3-(4- methylpiperazin-1-yl)propan- amido]pyridin-4-yl}amino)- pyridazin-3-yl]azetidine-2- carboxylate |
| 151 | 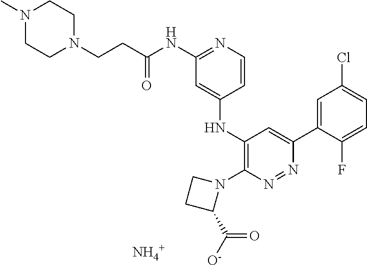 [SMILES: CN1CCN(CCC(=O)Nc2cc(Nc3cc(-c4cc(Cl)ccc4F)nnc3N3CC[C@H]3C(=O)[O-])ccn2)CC1 ; [NH4+]] | ammonium 1-[6-(5-chloro-2- fluorophenyl)-4-({2-[3-(4- methylpiperazin-1-yl)propan- amido]pyridin-4-yl}amino)- pyridazin-3-yl]azetidine-2- carboxylate |
| 152 | 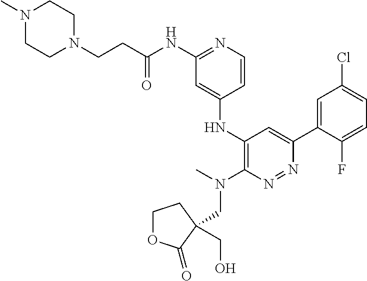 [SMILES: CN1CCN(CCC(=O)Nc2cc(Nc3cc(-c4cc(Cl)ccc4F)nnc3N(C)C[C@@]3(CO)CCOC3=O)ccn2)CC1] | N-(4-{[6-(5-chloro-2-fluoro- phenyl)-3-({[3-(hydroxymeth- yl)-2-oxooxolan-3-yl]methyl}- (methyl)amino)pyridazin-4- yl]amino}pyridin-2-yl)-3-(4- methylpiperazin-1-yl)propan- amide |
| 153 | 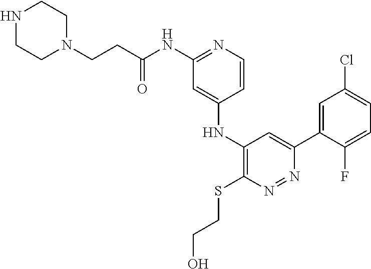 [SMILES: O=C(CCN1CCNCC1)Nc1cc(Nc2cc(-c3cc(Cl)ccc3F)nnc2SCCO)ccn1] | N-(4-{[6-(5-chloro-2-fluoro- phenyl)-3-[(2-hydroxyethyl)- sulfanyl]pyridazin-4-yl]amino}- pyridin-2-yl)-3-(piperazin-1- yl)propanamide |
| 154 | 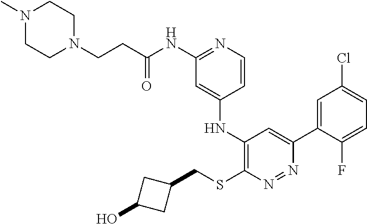 | N-(4-{[6-(5-chloro-2-fluoro- phenyl)-3-{[(3-hydroxycyclo- butyl)methyl]suIfanyl}pyrid- azin-4-yl]amino}pyridin-2-yl)- 3-(4-methylpiperazin-1-yl)- propanamide |
| 155 | 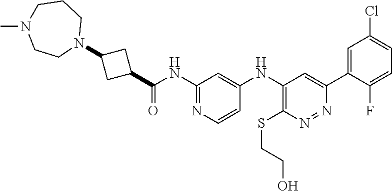 | Cis N-(4-{[6-(5-chloro-2- fluorophenyl)-3-[(2-hydroxy- ethyl)sulfanyl]pyridazin-4- yl]amino}pyridin-2-yl)-3-(4- methyl-1,4-diazepan-1-yl)- cyclobutane-1-carboxamide |
| 156 | 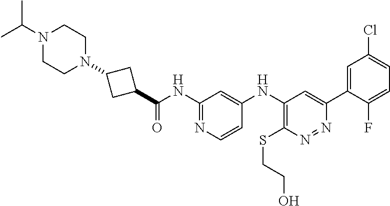 | Trans N-(4-{[6-(5-chloro-2- fluorophenyl)-3-[(2-hydroxy- ethyl)sulfanyl]pyridazin-4- yl]amino}pyridin-2-yl)-3-[4- (propan-2-yl)piperazin-1-yl]- cyclobutane-1-carboxamide |
| 157 | 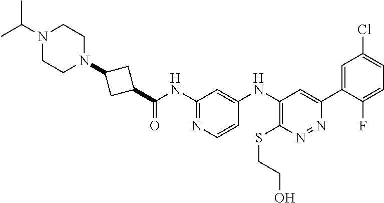 | Cis N-(4-{[6-(5-chloro-2- fluorophenyl)-3-[(2-hydroxy- ethyl)sulfanyl]pyridazin-4- yl]amino}pyridin-2-yl)-3-[4- (propan-2-yl)piperazin-1-yl]- cyclobutane-1-carboxamide |
| 158 | 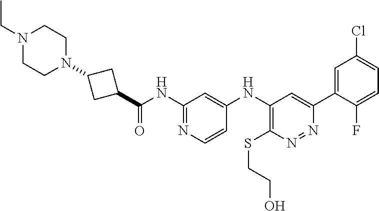 | Trans N-(4-{[6-(5-chloro-2- fluorophenyl)-3-[(2-hydroxy- ethyl)sulfanyl]pyridazin-4-yl]- amino}pyridin-2-yl)-3-(4- ethylpiperazin-1-yl)cyclo- butane-1-carboxamide |
| 159 | 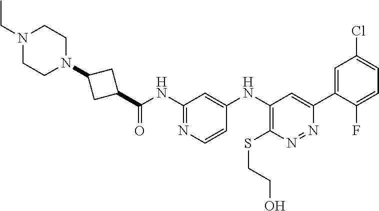 | Cis N-(4-{[6-(5-chloro-2- fluorophenyl)-3-[(2-hydroxy- ethyl)sulfanyl]pyridazin-4-yl]- amino}pyridin-2-yl)-3-(4- ethylpiperazin-1-yl)cyclo- butane-1-carboxamide |
| 160 | 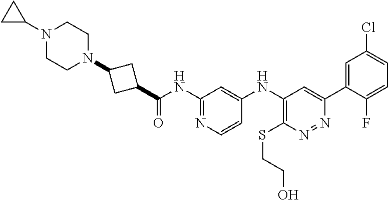 | Cis N-(4-{[6-(5-chloro-2- fluorophenyl)-3-[(2-hydroxy- ethyl)sulfanyl]pyridazin-4-yl]- amino}pyridin-2-yl)-3-(4- cyclopropylpiperazin-1-yl)- cyclobutane-1-carboxamide |
| 161 | 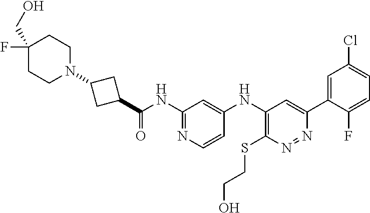 | Trans N-(4-{[6-(5-chloro-2- fluorophenyl)-3-[(2-hydroxy- ethyl)sulfanyl]pyridazin-4-yl]- amino}pyridin-2-yl)-3-[4- fluoro-4-(hydroxymethyl)- piperidin-1-yl]cyclobutane-1- carboxamide |
| 162 | 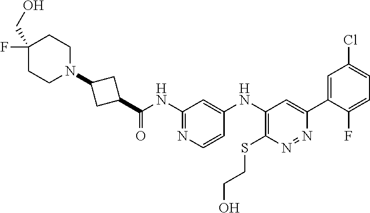 | Cis N-(4-{[6-(5-chloro-2- fluorophenyl)-3-[(2-hydroxy- ethyl)sulfanyl]pyridazin-4-yl]- amino}pyridin-2-yl)-3-[4- fluoro-4-(hydroxymethyl)- piperidin-1-yl]cyclobutane-1- carboxamide |
| 163 | 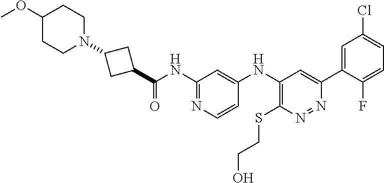 | Trans N-(4-{[6-(5-chloro-2- fluorophenyl)-3-[(2-hydroxy- ethyl)sulfanyl]pyridazin-4-yl]- amino}pyridin-2-yl)-3-(4- methoxypiperidin-1-yl)cyclo- butane-1-carboxamide |
| 164 | 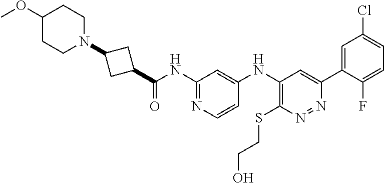 | Cis N-(4-{[6-(5-chloro-2- fluorophenyl)-3-[(2-hydroxy- ethyl)sulfanyl]pyridazin-4-yl]- amino}pyridin-2-yl)-3-(4- methoxypiperidin-1-yl)cyclo- butane-1-carboxamide |
| 165 | 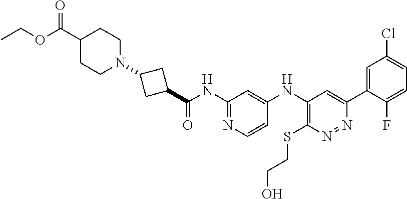 | Trans ethyl 1-{3-[(4-{[6-(5- chloro-2-fluorophenyl)-3- [(2-hydroxyethyl)sulfanyl]- pyridazin-4-yl]amino}pyridin- 2-yl)carbamoyl]cyclobutyl}- piperidine-4-carboxylate |
| 166 | 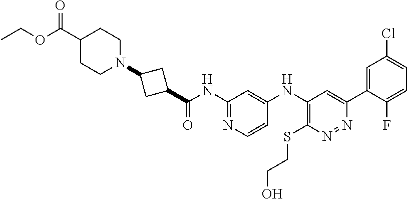 | Cis ethyl 1-{3-[(4-{[6-(5- chloro-2-fluorophenyl)-3-[(2- hydroxyethyl)sulfanyl]pyr- idazin-4-yl]amino}pyridin-2- yl)carbamoyl]cyclobutyl}- piperidine-4-carboxylate |
| 167 | 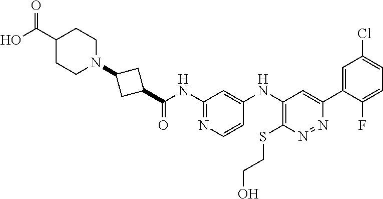 | Cis 1-{3-[(4-{[6-(5-chloro-2- fluorophenyl)-3-[(2-hydroxy- ethyl)sulfanyl]pyridazin-4-yl]- amino}pyridin-2-l)carbamoyl]- cyclobutyl}piperidine-4-car- boxylic acid |
| 168 | 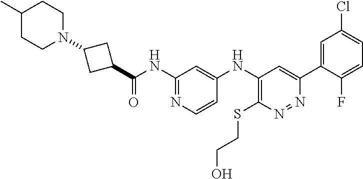 | Trans N-(4-{[6-(5-chloro-2- fluorophenyl)-3-[(2-hydroxy- ethyl)sulfanyl]pyridazin-4-yl]- amino}pyridin-2-yl)-3-(4- methylpiperidin-1-yl)cyclo- butane-1-carboxamide |
| 169 | 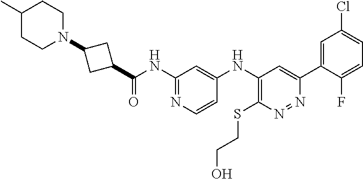 | Cis N-(4-{[6-(5-chloro-2- fluorophenyl)-3-[(2-hydroxy- ethyl)sulfanyl]pyridazin-4-yl]- amino}pyridin-2-yl)-3-(4- methylpiperidin-1-yl)cyclo- butane-1-carboxamide |
| 170 | 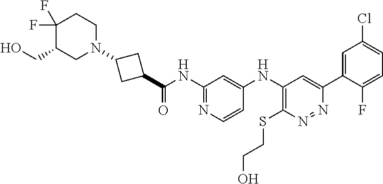 | Trans N-(4-{[6-(5-chloro-2- fluorophenyl)-3-[(2-hydroxy- ethyl)sulfanyl]pyridazin-4-yl]- amino}pyridin-2-yl)-3-[4,4- difluoro-3-(hydroxymethyl)- piperidin-1-yl]cyclobutane-1- carboxamide |
| 171 | 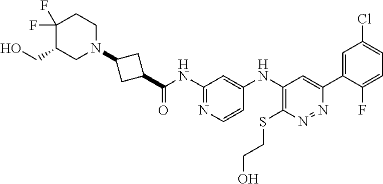 | Cis N-(4-{[6-(5-chloro-2- fluorophenyl)-3-[(2-hydroxy- ethyl)sulfanyl]pyridazin-4-yl]- amino}pyridin-2-yl)-3-[4,4- difluoro-3-(hydroxymethyl)- piperidin-1-yl]cyclobutane-1- carboxamide |
| 172 | 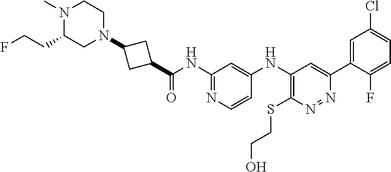 | Cis N-(4-{[6-(5-chloro-2- fluorophenyl)-3-[(2-hydroxy- ethyl)sulfanyl]pyridazin-4-yl]- amino}pyridin-2-yl)-3-[3-(2- fluoroethyl)-4-methylpiper- azin-1-yl]cyclobutane-1-car- boxamide |
| 173 | 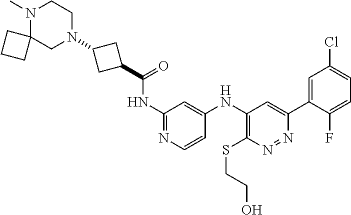 | Trans N-(4-{[6-(5-chloro-2- fluorophenyl)-3-[(2-hydroxy- ethyl)sulfanyl]pyridazin-4-yl]- amino}pyridin-2-yl)-3-{5- methyl-5,8-diazaspiro[3.5]- nonan-8-yl}cyclobutane-1- carboxamide |
| 174 | 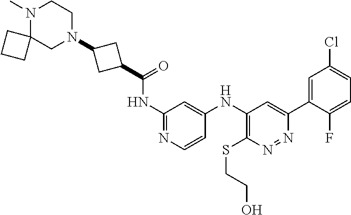 | Cis N-(4-{[6-(5-chloro-2- fluorophenyl)-3-[(2-hydroxy- ethyl)sulfanyl]pyridazin-4-yl]- amino}pyridin-2-yl)-3-{5- methyl-5,8-diazaspiro[3.5]- nonan-8-yl}cyclobutane-1- carboxamide |
| 175 | 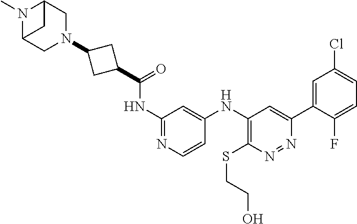 | Cis N-(4-{[6-(5-chloro-2- fluorophenyl)-3-[(2-hydroxy- ethyl)sulfanyl]pyridazin-4-yl]- amino}pyridin-2-yl)-3-{6- methyl-3,6-diazabicyclo- [3.1.1]heptan-3-cyclobutane- 1-carboxamide |
| 178 | 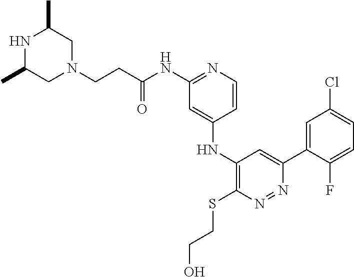 [SMILES: C[C@@H]1CN(CCC(=O)Nc2cc(Nc3cc(-c4cc(Cl)ccc4F)nnc3SCCO)ccn2)C[C@H](C)N1] | N-(4-{[6-(5-chloro-2-fluoro- phenyl)-3-[(2-hydroxyethyl)- sulfanyl]pyridazin-4-yl]amino}- pyridin-2-yl)-3-(3,5-dimethyl- piperazin-1-yl)propanamide |
| 179 | 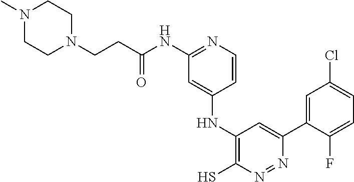 [SMILES: CN1CCN(CCC(=O)Nc2cc(Nc3cc(-c4cc(Cl)ccc4F)nnc3S)ccn2)CC1] | N-(4-{[6-(5-chloro-2-fluoro- phenyl)-3-sulfanylpyridazin-4- yl]amino}pyridin-2-yl)-3-(4- methylpiperazin-1-yl)propan- amide |
| 180 | 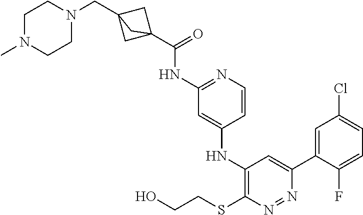 [SMILES: CN1CCN(CC23CC(C(=O)Nc4cc(Nc5cc(-c6cc(Cl)ccc6F)nnc5SCCO)ccn4)(C2)C3)CC1] | N-(4-{[6-(5-chloro-2-fluoro- phenyl)-3-[(2-hydroxyethyl)- sulfanyl]pyridazin-4-yl]amino}- pyridin-2-yl)-3-[(4-methyl- piperazin-1-yl)methyl]bicyclo- [1.1.1]pentane-1-carboxamide |
| 183 | 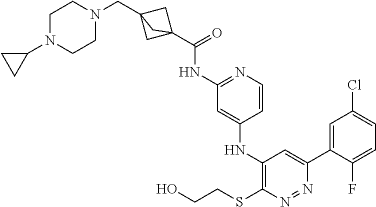 [SMILES: O=C(Nc1cc(Nc2cc(-c3cc(Cl)ccc3F)nnc2SCCO)ccn1)C12CC(CN3CCN(C4CC4)CC3)(C1)C2] | N-(4-{[6-(5-chloro-2-fluoro- phenyl)-3-[(2-hydroxyethyl)- sulfanyl]pyridazin-4-yl]amino}- pyridin-2-yl)-3-[(4-cyclo- propylpiperazin-1-yl)methyl]- bicyclo[1.1.1]pentane-1-car-boxamide |
| 184 | 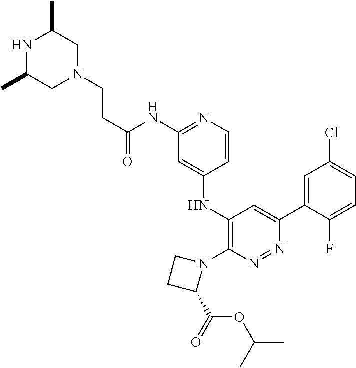 [SMILES: CC(C)OC(=O)[C@@H]1CCN1c1nnc(-c2cc(Cl)ccc2F)cc1Nc1ccnc(NC(=O)CCN2C[C@@H](C)N[C@@H](C)C2)c1] | propan-2-yl 1-[6-(5-chloro-2- fluorophenyl)-4-({2-[3-(3,5- dimethylpiperazin-1-yl)propan- amido]pyridin-4-yl}amino)- pyridazin-3-yl]azetidine-2- carboxylate |
| 188 | 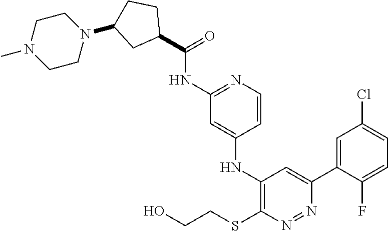 | Cis Enantiomer 1 N-(4-{[6-(5- chloro-2-fluorophenyl)-3-[(2- hydroxyethyl)sulfanyl]pyrid- azin-4-yl]amino}pyridin-2-yl)- 3-(4-methylpiperazin-1-yl)- cyclopentane-1-carboxamide |
| 189 | 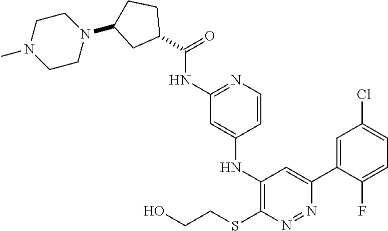 | Trans Enantiomer 1 N-(4-{[6- (5-chloro-2-fluorophenyl)-3- [(2-hydroxyethyl)sulfanyl]- pyridazin-4-yl]amino}pyridin- 2-yl)-3-(4-methylpiperazin-1- yl)cyclopentane-1-carboxamide |
| 190 | 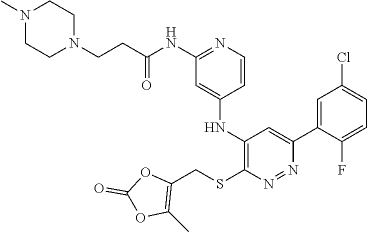 [SMILES: Cc1oc(=O)oc1CSc1nnc(-c2cc(Cl)ccc2F)cc1Nc1ccnc(NC(=O)CCN2CCN(C)CC2)c1] | N-(4-{[6-(5-chloro-2-fluoro- phenyl)-3-{[(5-methyl-2-oxo- 2H-1,3-dioxol-4-yl)methyl]- sulfanyl}pyridazin-4-yl]- amino}pyridin-2-yl)-3-(4- methylpiperazin-1-yl)propan- amide |
| 191 | 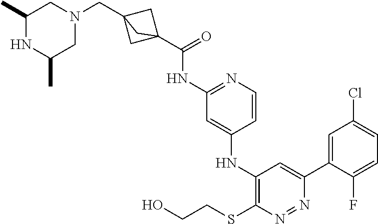 [SMILES: C[C@@H]1CN(CC23CC(C(=O)Nc4cc(Nc5cc(-c6cc(Cl)ccc6F)nnc5SCCO)ccn4)(C2)C3)C[C@H](C)N1] | N-(4-{[6-(5-chloro-2-fluoro- phenyl)-3-[(2-hydroxyethyl)- sulfanyl]pyridazin-4-yl]amino}- pyridin-2-yl)-3-[(3,5-dimethyl- piperazin-1-yl)methyl]bicyclo- [1.1.1]pentane-1-carboxamide |
| 192 | 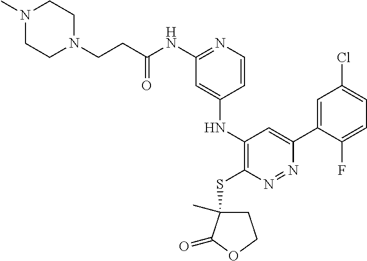 [SMILES: CN1CCN(CCC(=O)Nc2cc(Nc3cc(-c4cc(Cl)ccc4F)nnc3S[C@]3(C)CCOC3=O)ccn2)CC1] | N-(4-{[6-(5-chloro-2-fluoro- phenyl)-3-[(3-methyl-2-oxo- oxolan-3-yl)sulfanyl]pyridazin- 4-yl]amino}pyridin-2-yl)-3-(4- methylpiperazin-1-yl)propan- amide |
| 193 | 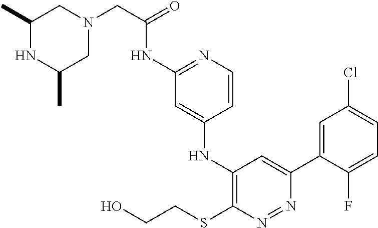 [SMILES: C[C@@H]1CN(CC(=O)Nc2cc(Nc3cc(-c4cc(Cl)ccc4F)nnc3SCCO)ccn2)C[C@H](C)N1] | N-(4-{[6-(5-chloro-2-fluoro- phenyl)-3-[(2-hydroxyethyl)- sulfanyl]pyridazin-4-yl]amino}- pyridin-2-yl)-2-(3,5-dimethyl- piperazin-1-yl)acetamide |
| 194 | 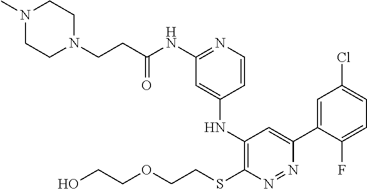 [SMILES: CN1CCN(CCC(=O)Nc2cc(Nc3cc(-c4cc(Cl)ccc4F)nnc3SCCOCCO)ccn2)CC1] | N-(4-{[6-(5-chloro-2-fluoro- phenyl)-3-{[2-(2-hydroxy- ethoxy)ethyl]sulfanyl}pyrid- azin-4-yl]amino}pyridin-2-yl)- 3-(4-methylpiperazin-1-yl)- propanamide |
| 196 | 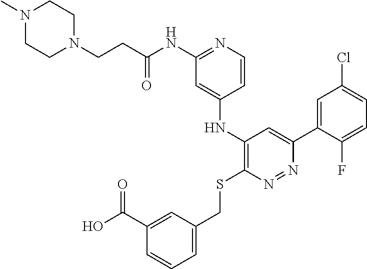 [SMILES: CN1CCN(CCC(=O)Nc2cc(Nc3cc(-c4cc(Cl)ccc4F)nnc3SCc3cccc(C(=O)O)c3)ccn2)CC1] | 3-({[6-(5-chloro-2-fluorophen- yl)-4-({2-[3-(4-methylpiper- azin-1-yl)propanamido]pyridin- 4-yl}amino)pyridazin-3-yl]- sulfanyl}methyl)benzoic acid |
| 197 | 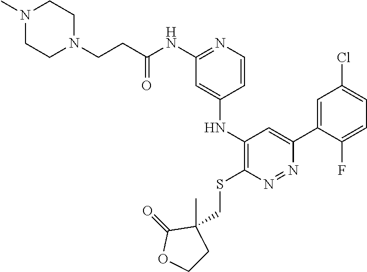 [SMILES: CN1CCN(CCC(=O)Nc2cc(Nc3cc(-c4cc(Cl)ccc4F)nnc3SC[C@]3(C)CCOC3=O)ccn2)CC1] | N-(4-{[6-(5-chloro-2-fluoro- phenyl)-3-{[(3-methyl-2-oxo- oxolan-3-yl)methyl]sulfanyl}- pyridazin-4-yl]amino}pyridin- 2-yl)-3-(4-methylpiperazin-1- yl)propanamide |
| 200 | 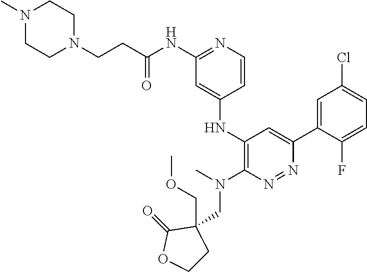 [SMILES: COC[C@@]1(CN(C)c2nnc(-c3cc(Cl)ccc3F)cc2Nc2ccnc(NC(=O)CCN3CCN(C)CC3)c2)CCOC1=O] | N-(4-{[6-(5-chloro-2-fluoro- phenyl)-3-({[3-(methoxymeth- yl)-2-oxooxolan-3-yl]methyl}- (methyl)amino)pyridazin-4- yl]amino}pyridin-2-yl)-3-(4- methylpiperazin-1-yl)propan- amide |
| 201 | 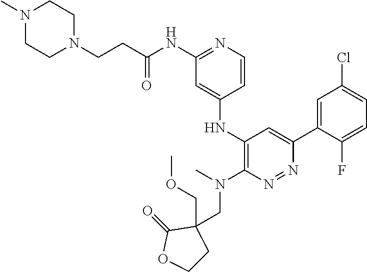 [SMILES: COCC1(CN(C)c2nnc(-c3cc(Cl)ccc3F)cc2Nc2ccnc(NC(=O)CCN3CCN(C)CC3)c2)CCOC1=O] | Enantiomer 1 N-(4-{[6-(5- chloro-2-fluorophenyl)-3- ({[3-(methoxymethyl)-2-oxo- oxolan-3-yl]methyl}(methyl)- amino)pyridazin-4-yl]amino}- pyridin-2-yl)-3-(4-methylpiper- azin-1-yl)propanamide |
| 202 | 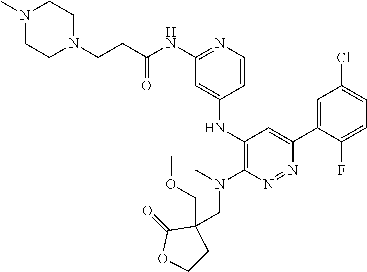 [SMILES: COCC1(CN(C)c2nnc(-c3cc(Cl)ccc3F)cc2Nc2ccnc(NC(=O)CCN3CCN(C)CC3)c2)CCOC1=O] | Enantiomer 2 N-(4-{[6-(5- chloro-2-fluorophenyl)-3- ({[3-(methoxymethyl)-2-oxo- oxolan-3-yl]methyl}(methyl)- amino)pyridazin-4-yl]amino}- pyridin-2-yl)-3-(4-methylpiper- azin-1-yl)propanamide |
| 203 | 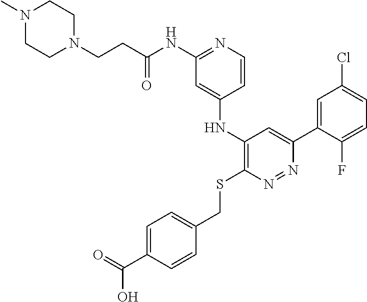 [SMILES: CN1CCN(CCC(=O)Nc2cc(Nc3cc(-c4cc(Cl)ccc4F)nnc3SCc3ccc(C(=O)O)cc3)ccn2)CC1] | N-(4-{[6-(5-chloro-2-fluoro- phenyl)-3-({[3-(methoxymeth- yl)-2-oxooxolan-3-yl]methyl}- (methyl)amino)pyridazin-4- yl]amino}pyridin-2-yl)-3-(4- methylpiperazin-1-yl)propan- amide |
| 204 | 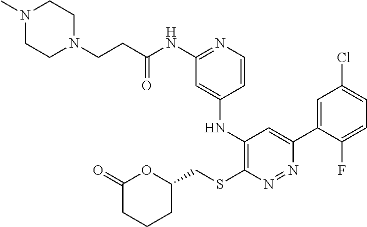 [SMILES: CN1CCN(CCC(=O)Nc2cc(Nc3cc(-c4cc(Cl)ccc4F)nnc3SC[C@@H]3CCCC(=O)O3)ccn2)CC1] | N-(4-{[6-(5-chloro-2-fluoro- phenyl)-3-{[(6-oxooxan-2-yl)- methyl]sulfanyl}pyridazin-4- yl]amino}pyridin-2-yl)-3-(4- methylpiperazin-1-yl)propan- amide |
| 205 | 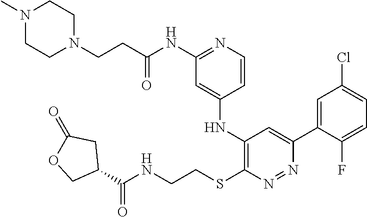 [SMILES: CN1CCN(CCC(=O)Nc2cc(Nc3cc(-c4cc(Cl)ccc4F)nnc3SCCNC(=O)[C@@H]3COC(=O)C3)ccn2)CC1] | N-(2-{[6-(5-chloro-2-fluoro- phenyl)-4-({2-[3-(4-methyl- piperazin-1-yl)propanamido]- pyridin-4-yl}amino)pyridazin- 3-yl]sulfanyl}ethyl)-5-oxo- oxolane-3-carboxamide |

In a even more preferred embodiment the present invention refers to a compound of formula (I), wherein A is Ala

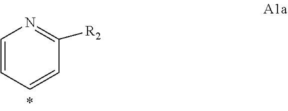

represented by the formula (Iaa)

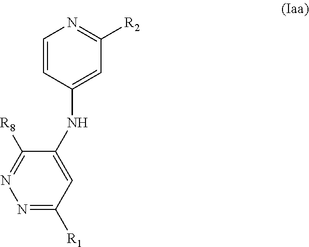

R₁ is selected from the group consisting of aryl and pyridyl, wherein said aryl and pyridyl are optionally substituted by one or more groups selected from —(C₁-C₆)alkyl and halogen atoms;
R₂ is selected from the group consisting of —NR₅C(O)R₆, —NR₅R₉ and —NH₂;
R₅ is H or —(C₁-C₆)alkyl;
R₆ is selected from the group consisting of —(C₃-C₉)heterocycloalkyl substituted by one or more —(C₁₋C₆)alkyl; —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl and —(C₃-C₆)cycloalkyl; —(C₁-C₆)alkylene-NH₂; —(C₃-C₆)cycloalkyl optionally substituted by one or more —(C₁-C₆)alkylene-(C₃-C₉)heterocycloalkyl, wherein said —(C₃-C₉)heterocycloalkyl is optionally substituted by one or more groups selected from —(C₁-C₆)alkyl and —(C₃-C₆)cycloalkyl; and —(C₃-C₆)cycloalkyl optionally substituted by one or more —(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —O—(C₁-C₆)alkyl, —C(O)OH, —C(O)O—(C₁-C₆)alkyl, —(C₁-C₆)haloalkyl and halogen atoms;
R₈ is selected from the group consisting of —NR_{A}R_{B}; —SH; —S—(C₁-C₆)alkyl, wherein said —(C₁-C₆)alkyl is optionally substituted by one or more —OH; —S—(C₁-C₆)alkylene-OH; —S—(C₃-C₉)heterocycloalkyl, wherein said —(C₃-C₉)heterocycloalkyl is optionally substituted by one or more groups selected from —(C₁-C₆)alkyl and oxo; —S—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —S(O)=NH—(C₁-C₆)alkyl; —S(O)₂—(C₁-C₆)alkyl; —S(O)—(C₁-C₆)alkyl; S—(C₁-C₆)alkylene-(C₃-C₆)cycloalkyl, wherein said —(C₃-C₆)cycloalkyl is optionally substituted by one or more —OH; —O—(C₁-C₆)alkyl; S—(C₁-C₆)alkylene-aryl, wherein said aryl is optionally substituted by one or more —C(O)OH; —O—(C₁-C₆)haloalkyl; —S—(C₁-C₆)alkylene-O—(C₁-C₆)alkylene-OH; —O—(C₁-C₆)alkylene-OH, wherein said —O—(C₁-C₆)alkylene is substituted by one or more —OH; —O—(C₁-C₆)alkylene-C(O)O—(C₁-C₆)alkyl; —S—(C₁-C₆)alkylene-NH—C(O)—(C₃-C₉)heterocycloalkyl, wherein said —(C₃-C₉)heterocycloalkyl is optionally substituted by one or more oxo; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁₋C₆)alkylene-S(O)—(C₁-C₆)alkyl; —O—(C₁-C₆)alkylene-S(O)₂—(C₁-C₆)alkyl; —O—(C₁-C₆)alkylene-NH—S(O)₂—(C₁-C₆)alkyl; —O—(C₁-C₆)alkylene-O—(C₁-C₆)alkyl; —O—(C₁-C₆)alkylene-(C₃-C₆)cycloalkyl, wherein said —(C₃-C₆)cycloalkyl is optionally substituted by one or more groups selected from —(C₁-C₆)alkyl, —(C₁-C₆)alkylene-OH, —C(O)O—(C₁-C₆)alkyl and —OH; —O—(C₁-C₆)alkylene-aryl, wherein said aryl is optionally substituted by one or more —OH; —O—(C₁-C₆)alkylene-aryl, wherein said aryl is fused to a —(C₅-C₆)heterocycloalkyl, wherein said —(C₅-C₆)heterocycloalkyl is optionally substitued by one or more groups selected from oxo and —(C₁-C₆)alkyl; —(C₃-C₉)heterocycloalkyl optionally substituted by one or more groups selected from —C(O)OH and —C(O)O—(C₁-C₆)alkyl; —O—(C₃-C₉)heterocycloalkyl; and —O—(C₁-C₆)alkylene-(C₃-C₉)heterocycloalkyl, wherein said —(C₃-C₉)heterocycloalkyl is optionally substituted by one or more —(C₁-C₆)alkyl and oxo;
R₉ is a heteroaryl optionally substituted by one or more groups selected from —C(O)O—(C₁-C₆)alkyl and —(C₃-C₉)heterocycloalkyl, wherein said —(C₃-C₉)heterocycloalkyl is optionally substituted by one or more —(C₁-C₆)alkyl;
R_{A} is H or —(C₁₋C₆)alkyl;
R_{B} is H or selected from the group consisting of —(C₁-C₆)alkyl optionally substituted by one or more groups selected from halogen and —OH; —S(O)₂—(C₁-C₆)alkyl; —(C₁-C₆)alkylene-aryl, wherein said aryl is susbtituted by —OH; —(C₃-C₉)heterocycloalkyl; —(C₁-C₆)alkylene-C(O)O—(C₁-C₆)alkyl; and —(C₁-C₆)alkylene-(C₃-C₉)heterocycloalkyl, wherein said —(C₃-C₉)heterocycloalkyl is optionally substituted by one or more groups selected from —(C₁-C₆)alkyl, —(C₁-C₆)alkylene-OH, —(C₁-C₆)alkylene-O—(C₁-C₆)alkyl and oxo; or alternatively R_{A} and R_{B} together with the nitrogen atom to which they are attached may form a —(C₃-C₆)heterocycloalkyl, wherein said —(C₃-C₆)heterocycloalkyl is optionally substituted by one or more groups selected from —C(O)OH, —(C₁-C₆)alkylene-OH, —C(O)O—(C₁-C₆)alkyl and oxo;
R_{C} is —(C₁-C₆)alkyl; and pharmaceutically acceptable salts thereof.

In a more preferred embodiment the present invention refers to a compound of formula (Iaa), wherein R₂ is —NR₅C(O)R₆, R₅ is H or —(C₁-C₆)alkyl, R₆ is selected from the group consisting of -(4-methylpiperazin-1-yl)ethyl, -[4-(2-aminoethyl)piperazin-1-yl]-ethyl, methyl (2-(4-ethylpiperazin-1-yl)ethyl)carbamate, methyl 4-ethyl-1-methylpiperazine-2-carboxylate, -[4-(2,2,2-trifluoroethyl)piperazin-1-yl]ethyl, -[4-(2,2,2-trifluoroethyl)piperazin-1-yl]methyl, -(4-methylpiperazin-1-yl)propyl, -(6-methyl-2,6-diazaspiro[3.3]heptan-2-yl)methyl, -(5-methyl-2,5-diazabicyclo[2.2.1]heptan-2-yl)methyl, -2-methyl-2,8-diazaspiro[4.5]decane, -(4-methyl-1,4-diazepan-1-yl)methyl, -(morpholin-4-yl)ethyl, cyclopropyl, -(piperazin-1-yl)methyl, -((4-methyl-1,4-diazepan-1-yl)methyl), -(2-(piperazin-1-yl)ethyl), -((6-methyl-3,6-diazabicyclo[3.2.2]nonan-3-yl)methyl), -(3-(4-methylpiperazin-1-yl)cyclobutyl), -(2-(4-methyl-1,4-diazepan-1-yl)ethyl), -3-[(1S,4S)-5-methyl-2,5-diazabicyclo[2.2.1]heptan-2-yl]cyclobutyl, -3-(thiomorpholin-4-yl)cyclobutyl, -3-{4-methyl-4,7-diazaspiro[2.5]octan-7-yl}cyclobutyl, -[(1S,4S)-5-methyl-2,5-diazabicyclo[2.2.1]heptan-2-yl]methyl, -(2-(piperazin-1-yl)ethyl), -3-(4-methyl-1,4-diazepan-1-yl)cyclobutyl, -3-[4-(propan-2-yl)piperazin-1-yl]cyclobutyl, 3-(4-ethylpiperazin-1-yl)cyclobutyl, -3-(4-cyclopropylpiperazin-1-yl)cyclobutyl, -3-[4-fluoro-4-(hydroxymethyl)piperidin-1-yl]cyclobutyl, -3-(4-methoxypiperidin-1-yl)cyclobutyl, ethyl -(cyclobutyl)piperidine-4-carboxylate, -(cyclobutyl)piperidine-4-carboxylic acid, -3-(4-methylpiperidin-1-yl)cyclobutyl, -3-[4,4-difluoro-3-(hydroxymethyl)piperidin-1-yl]cyclobutyl, -3-[3-(2-fluoroethyl)-4-methylpiperazin-1-yl]cyclobutyl, -3-{5-methyl-5,8-diazaspiro[3.5]nonan-8-yl}cyclobutyl, -3-{6-methyl-3,6-diazabicyclo[3.1.1]heptan-3-yl}cyclobutyl, -(3,5-dimethylpiperazin-1-yl)ethyl, -3-[(4-methylpiperazin-1-yl)methyl]bicyclo[1.1.1]pentyl, -3-[(4-cyclopropylpiperazin-1-yl)methyl]bicyclo[1.1.1]pentyl, -3-(4-methylpiperazin-1-yl)cyclopentyl, -3-{[(3R,5S)-3,5-dimethylpiperazin-1-yl] methyl}bicyclo[1.1.1]pentyl and -(3,5-dimethylpiperazin-1-yl)methyl and R₈ is selected from the group consisting of -(2-hydroxyethoxy), -[3-(methylsulfanyl)propoxy], -(3-methanesulfonylpropoxy), -(2-aminoethoxy), -(2-methanesulfonamidoethoxy), -[2-(dimethylamino)ethoxy], -methoxy, methyl 2-methoxyacetate, -methylsulfanyl, -methanesulfinyl, -methanesulfonyl, methylsulfoximine, -[(2-hydroxyethyl)sulfanyl], -[(3-hydroxypropyl)sulfanyl], -(methylamino), -(dimethylamino), -(2-methoxyethoxy), -[2-(4-methylpiperazin-1-yl)ethoxy], -[2-(dimethylamino)ethoxy], -[(1-methylazetidin-3-yl)methoxy], -(2,2,2-trifluoroethoxy), -(2,2-difluoroethoxy), -[2-(pyrrolidin-1-yl)ethoxy], -(3-methanesulfinylpropoxy), -[3-(N,N,N-trimethylaminium)ethoxy], -[2-(4-methylpiperazin-1-yl)ethoxy], -(3-hydroxycyclobutyl)methoxy, -(tetrahydrofuran-3-yl)oxy, -[(2,2-dimethyl-1,3-dioxolan-4-yl)methoxy], -(2,3-dihydroxypropoxy), -[(2-oxo-1,3-dioxolan-4-yl)methoxy], -[3-(hydroxymethyl)cyclobutoxy], -[(3-hydroxyphenyl)methoxy], -[(1-hydroxy-2-methylpropan-2-yl)sulfanyl], -[3-(hydroxymethyl)azetidin-1-yl], methyl azetidine-3-carboxylate, azetidine-3-carboxylic acid, propan-2-yl azetidine-3-carboxylate, -{[(3-hydroxyphenyl)methyl]amino}, -{[(3-hydroxyphenyl)methyl](methyl)amino}, -{7-oxo-6-oxa-2-azaspiro[3.4]octan-2-yl}, -[methyl(oxolan-3-yl)amino], -{methyl[(2-oxooxolan-3-yl)methyl]amino}, -[methyl(4,4,4-trifluoro-3-hydroxybutyl)amino], -{[(2,2-dimethyl-1,3-dioxolan-4-yl)methyl]sulfanyl}, -[(2,2-dimethyl-2H-1,3-benzodioxol-5-yl)methoxy], -[(3-hydroxy-3-methylcyclobutyl)methoxy], -[(methyl bicyclo[1.1.1]pentane-1-carboxylate)methoxy], -{methyl[(3-methyl-2-oxooxolan-3-yl)methyl]amino}, methyl morpholine-2-carboxylate, morpholine-2-carboxylic acid, -{[ethyl -2,2-dimethylpropanoate](methyl)amino}, propan-2-yl azetidine-2-carboxylate, azetidine-2-carboxylic acid, -({[3-(hydroxymethyl)-2-oxooxolan-3-yl]methyl}(methyl)amino), -{[(3-hydroxycyclobutyl)methyl]sulfanyl}, -sulfanyl, -{[(5-methyl-2-oxo-2H-1,3-dioxol-4-yl)methyl]sulfanyl}, -[(3-methyl-2-oxooxolan-3-yl)sulfanyl], -{[2-(2-hydroxyethoxy)ethyl]sulfanyl}, -3-[(sulfanyl)methyl] benzoic acid, -{[(3-methyl-2-oxooxolan-3-yl)methyl]sulfanyl}, -({[3-(methoxymethyl)-2-oxooxolan-3-yl]methyl}(methyl)amino), -4-[(sulfanyl)methyl] benzoic acid, -{[(6-oxooxan-2-yl)methyl]sulfanyl} and N-[2-(sulfanyl)ethyl]-5-oxooxolane-3-carboxamide.

In a more preferred embodiment the present invention refers to a compound of formula (Iaa), wherein R₂ is —NR₅C(O)R₆, R₅ is H or —(C₁-C₆)alkyl, R₆ is selected from the group consisting of -(4-methylpiperazin-1-yl)ethyl, -(4-methyl-1,4-diazepan-1-yl)methyl, -(-3,5-dimethylpiperazin-1-yl)ethyl, -[(1S,4S)-5-methyl-2,5-diazabicyclo[2.2.1]heptan-2-yl]methyl and ethyl -(cyclobutyl)piperidine-4-carboxylate and R₈ is selected from the group consisting of -methylsulfanyl, -[(2-hydroxyethyl)sulfanyl], -[(2-oxo-1,3-dioxolan-4-yl)methoxy], -{[(6-oxooxan-2-yl)methyl]sulfanyl}, -{[(5-methyl-2-oxo-2H-1,3-dioxol-4-yl)methyl]sulfanyl}, -{[(3-methyl-2-oxooxolan-3-yl)methyl]sulfanyl} and N-[2-(sulfanyl)ethyl]-5-oxooxolane-3-carboxamide.

In another preferred embodiment the present invention refers to a compound of formula (Iaa), wherein R₂ is —NH₂.

In a equally preferred embodiment the present invention refers to a compound of formula (I), wherein A is A2

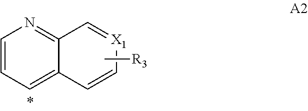

represented by the formula (Ib)

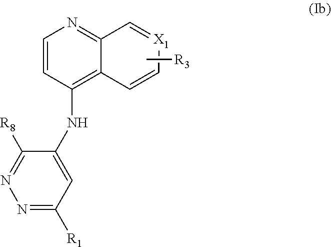

X₁ is C or CH;
R₃ is —OR₇;
R₇ is selected from the group consisting of —(C₁-C₆)alkyl and —(C₁-C₆)alkylene-(C₃-C₉)heterocycloalkyl, wherein said —(C₃-C₉)heterocycloalkyl is optionally substituted by one or more —(C₁-C₆)alkyl;
R₈ is selected from the group consisting of —NR_{A}R_{B}, —O—(C₁-C₆)alkyl, —O—(C₁-C₆)haloalkyl, —O—(C₁-C₆)alkylene-OH, wherein said —O—(C₁-C₆)alkylene is substituted by one or more —OH, —O—(C₁₋C₆)alkylene-C(O)O—(C₁₋C₆)alkyl, —O—(C₁-C₆)alkylene-NR_{A}R_{B}, —O—(C₁₋C₆)alkylene-N⁺R_{A}R_{B}R_{C}, —O—(C₁₋C₆)alkylene-S—(C₁₋C₆)alkyl, —O—(C₁-C₆)alkylene-S(O)—(C₁₋C₆)alkyl, —O—(C₁₋C₆)alkylene-S(O)₂—(C₁₋C₆)alkyl, —O—(C₁-C₆)alkylene-NH—S(O)₂—(C₁-C₆)alkyl, —O—(C₁₋C₆)alkylene-O—(C₁₋C₆)alkyl and —O—(C₁₋C₆)alkylene-(C₃-C₉)heterocycloalkyl, wherein said —(C₃-C₉)heterocycloalkyl is optionally substituted by one or more —(C₁-C₆)alkyl;
R_{A} is H or —(C₁₋C₆)alkyl;
R_{B} is H or selected from the group consisting of —(C₁-C₆)alkyl, —S(O)₂—(C₁-C₆)alkyl;
R_{C} is —(C₁₋C₆)alkyl; and pharmaceutically acceptable salts thereof.

In a particularly preferred embodiment the present invention refers to a compound of formula (Ib), wherein A is A2a

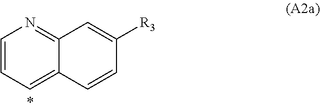

represented by the formula (Iba)

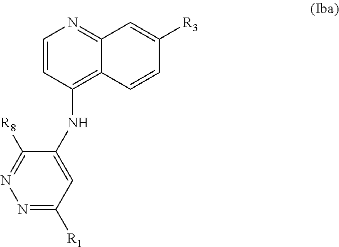

R₃ is —OR₇;
R₇ is selected from the group consisting of —(C₁₋C₆)alkyl and —(C₁₋C₆)alkylene-(C₃-C₉)heterocycloalkyl, wherein said —(C₃-C₉)heterocycloalkyl is optionally substituted by one or more —(C₁-C₆)alkyl;
R₈ is selected from the group consisting of —NR_{A}R_{B}, —S—(C₁-C₆)alkyl, —S—(C₁-C₆)alkylene-OH, —S(O)=NH—(C₁-C₆)alkyl, —S(O)₂—(C₁-C₆)alkyl, —S(O)—(C₁₋C₆)alkyl, —O—(C₁-C₆)alkyl, —O—(C₁-C₆)haloalkyl, —O—(C₁-C₆)alkylene-OH, wherein said —O—(C₁-C₆)alkylene is substituted by one or more —OH, —O—(C₁₋C₆)alkylene-C(O)O—(C₁-C₆)alkyl, —O—(C₁₋C₆)alkylene-NR_{A}R_{B}, —O—(C₁₋C₆)alkylene-N⁺R_{A}R_{B}R_{C}, —O—(C₁-C₆)alkylene-S—(C₁. C₆)alkyl, —O—(C₁₋C₆)alkylene-S(O)—(C₁₋C₆)alkyl, —O—(C₁₋C₆)alkylene-S(O)₂—(C₁-C₆)alkyl, —O—(C₁-C₆)alkylene-NH—S(O)₂—(C₁-C₆)alkyl, —O—(C₁₋C₆)alkylene-O—(C₁-C₆)alkyl and —O—(C₁₋C₆)alkylene-(C₃-C₉)heterocycloalkyl, wherein said —(C₃-C₉)heterocycloalkyl is optionally substituted by one or more —(C₁-C₆)alkyl;
R_{A} is H or —(C₁-C₆)alkyl;
R_{B} is H or selected from the group consisting of —(C₁₋C₆)alkyl, —S(O)₂—(C₁-C₆)alkyl;
R_{C} is —(C₁-C₆)alkyl; and pharmaceutically acceptable salts thereof.

In a more preferred embodiment the present invention refers to a compound of formula (Iba), wherein R₃ is —OR₇, R₇ is selected from the group consisting of methyl and -7-[2-(4-methylpiperazin-1-yl)ethoxy]quinolin-4-yl and R₈ is is selected from the group consisting of methoxy, -(2-hydroxyethoxy), -(2,2-difluoroethoxy), -(2-aminoethoxy), -(2-methanesulfonamidoethoxy), -(2-methoxyethoxy), -[2-(4-methylpiperazin-1-yl)ethoxy], -[2-(dimethylamino)ethoxy] and -(2,2,2-trifluoroethoxy).

According to a preferred embodiment, the invention refers to at least one of the compounds of Formula (Iba) listed in the Table 2 below and pharmaceutical acceptable salts thereof. These compounds are particularly active on receptor ALK5, as shown in Table 4.

TABLE 2
List of preferred compounds of Formula (Iba)

| Example | | |
|---|---|---|
| No | Structure | Chemical name |
| 2 | 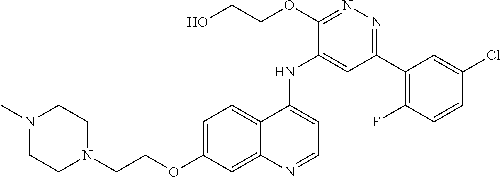 [SMILES: CN1CCN(CCOc2ccc3c(Nc4cc(-c5cc(Cl)ccc5F)nnc4OCCO)ccnc3c2)CC1] | 2-{[6-(5-chloro-2-fluorophenyl)- 4-({7-[2-(4-methylpiperazin-1- yl)ethoxy]quinolin-4- yl}amino)pyridazin-3- yl]oxy}ethan-1-ol |
| 3 | 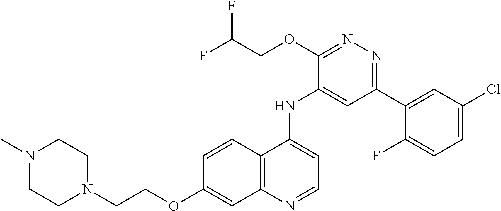 [SMILES: CN1CCN(CCOc2ccc3c(Nc4cc(-c5cc(Cl)ccc5F)nnc4OCC(F)F)ccnc3c2)CC1] | N-[6-(5-chloro-2-fluorophenyl)- 3-(2,2-difluoroethoxy)pyridazin- 4-yl]-7-[2-(4-methylpiperazin-1- yl)ethoxy]quinolin-4-amine |
| 5 | 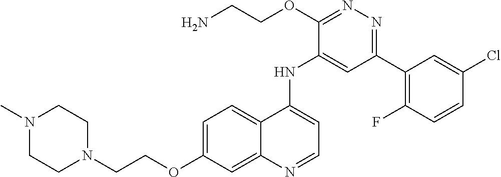 [SMILES: CN1CCN(CCOc2ccc3c(Nc4cc(-c5cc(Cl)ccc5F)nnc4OCCN)ccnc3c2)CC1] | N-[3-(2-aminoethoxy)-6-(5- chloro-2- fluorophenyl)pyridazin-4-yl]-7- [2-(4-methylpiperazin-1- yl)ethoxy\|quinolin-4-amine |
| 6 | 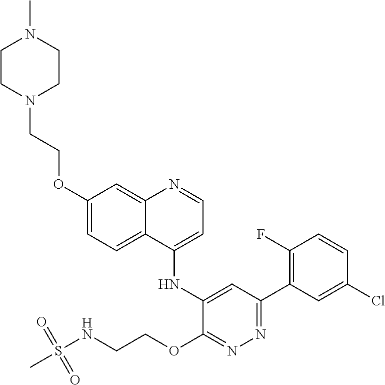 [SMILES: CN1CCN(CCOc2ccc3c(Nc4cc(-c5cc(Cl)ccc5F)nnc4OCCNS(C)(=O)=O)ccnc3c2)CC1] | N-(2-{[6-(5-chloro-2- fluorophenyl)-4-({7-[2-(4- methylpiperazin-1- yl)ethoxy]quinolin-4- yl}amino)pyridazin-3- yl]oxy}ethyl)methanesulfonamide |
| 32 | 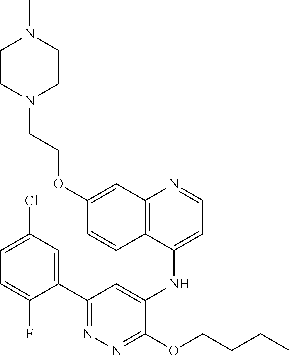 [SMILES: CCCCOc1nnc(-c2cc(Cl)ccc2F)cc1Nc1ccnc2cc(OCCN3CCN(C)CC3)ccc12] | N-[6-(5-chloro-2-fluorophenyl)- 3-(2-methoxyethoxy)pyridazin- 4-yl]-7-[2-(4-methylpiperazin-1- yl)ethoxy]quinolin-4-amine |
| 35 | 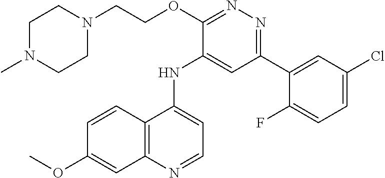 [SMILES: COc1ccc2c(Nc3cc(-c4cc(Cl)ccc4F)nnc3OCCN3CCN(C)CC3)ccnc2c1] | N-[6-(5-chloro-2-fluorophenyl)- 3-[2-(4-methylpiperazin-1- yl)ethoxy]pyridazin-4-yl]-7- methoxyquinolin-4-amine |
| 39 | 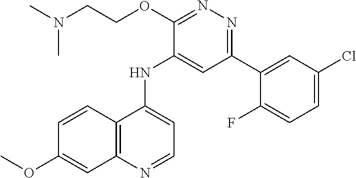 [SMILES: COc1ccc2c(Nc3cc(-c4cc(Cl)ccc4F)nnc3OCCN(C)C)ccnc2c1] | N-[6-(5-chloro-2-fluorophenyl)- 3-[2- (dimethylamino)ethoxy]pyridazin- 4-yl]-7-methoxyquinolin-4- amine |
| 46 | 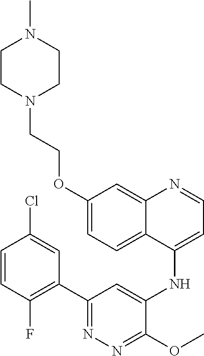 [SMILES: COc1nnc(-c2cc(Cl)ccc2F)cc1Nc1ccnc2cc(OCCN3CCN(C)CC3)ccc12] | N-[6-(5-chloro-2-fluorophenyl)- 3-methoxypyridazin-4-yl]-7-[2- (4-methylpiperazin-1- yl)ethoxy]quinolin-4-amine |
| 51 | 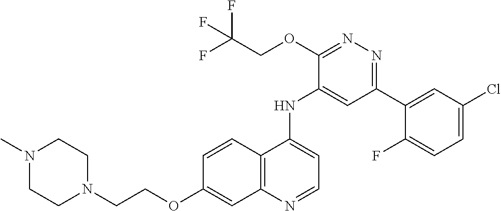 [SMILES: CN1CCN(CCOc2ccc3c(Nc4cc(-c5cc(Cl)ccc5F)nnc4OCC(F)(F)F)ccnc3c2)CC1] | N-[6-(5-chloro-2-fluorophenyl)- 3-(2,2,2- trifluoroethoxy)pyridazin-4-yl]- 7-[2-(4-methylpiperazin-1- yl)ethoxy]quinolin-4-amine |

According to another preferred embodiment, the present invention refers to a compound of formula (I), wherein A is A3

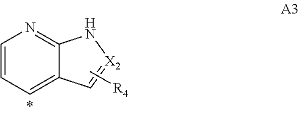

represented by the formula (Ic)

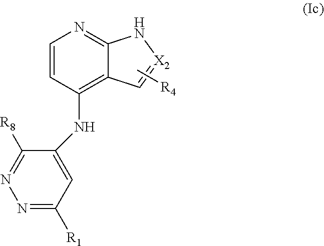

R₁ is selected from the group consisting of aryl and pyridyl, wherein said aryl and pyridyl are optionally substituted by one or more halogen atoms;
X₂ is C, CH or N;
R₄ is H or —C(O)O—(C₁-C₆)alkyl;
R₈ is selected from the group consisting of —NR_{A}R_{B}; —S—(C₁-C₆)alkylene-aryl, wherein said aryl is optionally substituted by one or more groups selected from —C(O)O—(C₁-C₆)alkylene-NR_{A}R_{C} and —C(O)O—(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; —O—(C₁-C₆)alkyl; —O—(C₁-C₆)haloalkyl; —O—(C₁-C₆)alkylene-OH, wherein said —O—(C₁-C₆)alkylene is substituted by one or more —OH; —O—(C₁-C₆)alkylene-C(O)O—(C₁-C₆)alkyl; —O—(C₁-C₆)alkylene-NR_{A}R_{B}; —O—(C₁-C₆)alkylene-N⁺R_{A}R_{B}R_{C}; —O—(C₁-C₆)alkylene-S—(C₁-C₆)alkyl; —O—(C₁-C₆)alkylene-S(O)—(C₁-C₆)alkyl; —O—(C₁-C₆)alkylene-S(O)₂—(C₁-C₆)alkyl; —O—(C₁-C₆)alkylene-O—(C₁-C₆)alkyl and —O—(C₁-C₆)alkylene-(C₃-C₉)heterocycloalkyl, wherein said —(C₃-C₉)heterocycloalkyl is optionally substituted by one or more —(C₁-C₆)alkyl;
R_{A} is H or —(C₁-C₆)alkyl;
R_{B} is H or selected from the group consisting of —(C₁-C₆)alkyl, —S(O)₂—(C₁-C₆)alkyl;
R_{C} is —(C₁-C₆)alkyl; and pharmaceutically acceptable salts thereof.

In a more preferred embodiment the present invention refers to a compound of formula (Ic), wherein R₈ is selected from the group consisting of -[3-(dimethylamino)propoxy], -[3-(N,N,N-trimethylamino)propoxy], -[2-(4-methylpiperazin-1-yl)ethoxy], -[2-(dimethylamino)ethoxy], (1-methylpiperidin-4-yl)methyl 4-[(sulfanyl)methyl]benzoate and 2-(dimethylamino)ethyl 4-[(sulfanyl)methyl]benzoate.

According to a preferred embodiment, the invention refers to at least one of the compounds of Formula (Ic) listed in the Table 3 below and pharmaceutical acceptable salts thereof. These compounds are particularly active on receptor ALK5, as shown in Table 4.

TABLE 3
List of preferred compounds of Formula (Ic)

| Example | | |
|---|---|---|
| No | Structure | Chemical name |
| 10 | 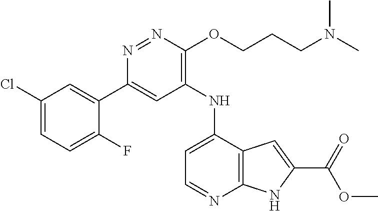 [SMILES: COC(=O)c1cc2c(Nc3cc(-c4cc(Cl)ccc4F)nnc3OCCCN(C)C)ccnc2[nH]1] | methyl 4-{[6-(5-chloro-2- fluorophenyl)-3-[3- (dimethylamino)propoxy]pyridazin-4- yl]amino}-1H-pyrrolo[2,3-b]pyridine- 2-carboxylate |
| 11 | 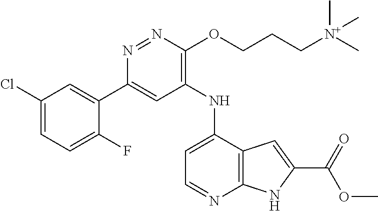 [SMILES: COC(=O)c1cc2c(Nc3cc(-c4cc(Cl)ccc4F)nnc3OCCC[N+](C)(C)C)ccnc2[nH]1] | (3-{[6-(5-chloro-2-fluorophenyl)-4- {[2-(methoxycarbonyl)-1H- pyrrolo[2,3-b]pyridin-4- yl]amino}pyridazin-3- yl]oxy}propyl)trimethylazanium chloride |
| 37 | 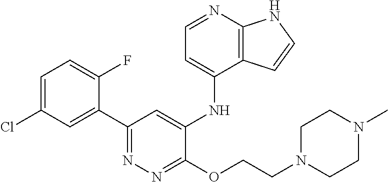 [SMILES: CN1CCN(CCOc2nnc(-c3cc(Cl)ccc3F)cc2Nc2ccnc3[nH]ccc23)CC1] | 6-(5-chloro-2-fluorophenyl)-3-[2-(4- methylpiperazin-1-yl)ethoxy]-N-{1H- pyrrolo[2,3-b]pyridin-4-yl}pyridazin- 4-amine |
| 41 | 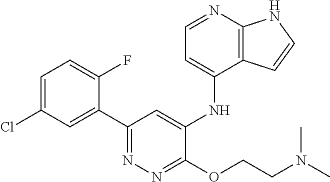 [SMILES: CN(C)CCOc1nnc(-c2cc(Cl)ccc2F)cc1Nc1ccnc2[nH]ccc12] | 6-(5-chloro-2-fluorophenyl)-3-[2- (dimethylamino)ethoxy]-N-{1H- pyrrolo[2,3-b]pyridin-4-yl}pyridazin- 4-amine |
| 42 | 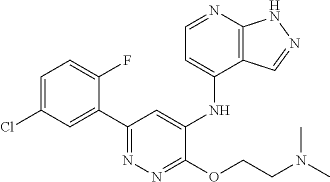 [SMILES: CN(C)CCOc1nnc(-c2cc(Cl)ccc2F)cc1Nc1ccnc2[nH]ncc12] | 6-(5-chloro-2-fluorophenyl)-3-[2- (dimethylamino)ethoxy]-N-{1H- pyrazolo[3,4-b]pyridin-4- yl}pyridazin-4-amine |
| 206 | 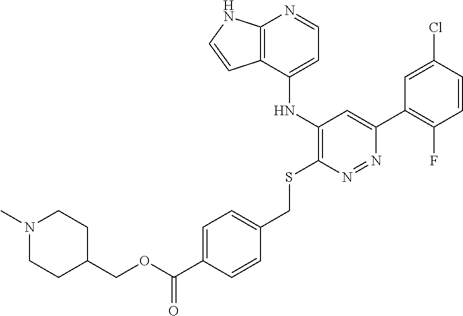 [SMILES: CN1CCC(COC(=O)c2ccc(CSc3nnc(-c4cc(Cl)ccc4F)cc3Nc3ccnc4[nH]ccc34)cc2)CC1] | (1-methylpiperidin-4-yl)methyl 3- ({[6-(5-chloro-2-fluorophenyl)-4- ({1H-pyrrolo[2,3-b]pyridin-4- yl}amino)pyridazin-3- yl]sulfanyl}methyl)benzoate |
| 207 | 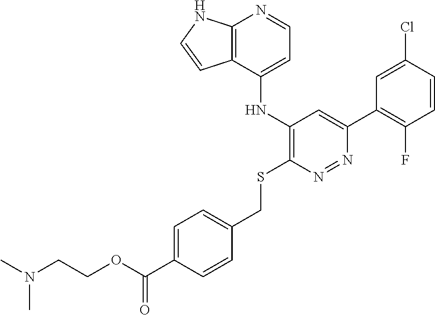 [SMILES: CN(C)CCOC(=O)c1ccc(CSc2nnc(-c3cc(Cl)ccc3F)cc2Nc2ccnc3[nH]ccc23)cc1] | 2-(dimethylamino)ethyl 3-({[6-(5- chloro-2-fluorophenyl)-4-({1H- pyrrolo[2,3-b]pyridin-4- yl}amino)pyridazin-3- yl]sulfanyl}methyl)benzoate |

In a particularly preferred embodiment the present invention refers to a compound of formula (Ic), wherein A is A3a

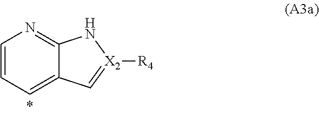

represented by the formula (Ica)

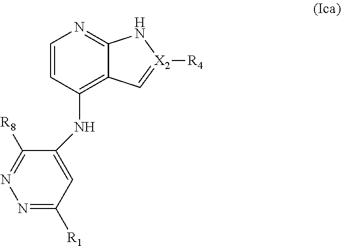

X₂ is C, R₄ is H or —C(O)O—(C₁-C₆)alkyl, and pharmaceutically acceptable salts thereof.

In a further preferred embodiment the present invention refers to a compound of formula (Ica), wherein R₄ is H.

In a further preferred embodiment the present invention refers to a compound of formula (Ica), wherein R₄ is methyl carboxylate.

In a further preferred embodiment the present invention refers to a compound of formula (Ica), wherein R₈ is selected from the group consisting of -[3-(dimethylamino)propoxy], -[3-(N,N,N-trimethylamino)propoxy], -[2-(4-methylpiperazin-1-yl)ethoxy], -[2-(dimethylamino)ethoxy], (1-methylpiperidin-4-yl)methyl 4-[(sulfanyl)methyl]benzoate and 2-(dimethylamino)ethyl 4-[(sulfanyl)methyl]benzoate.

In another particularly preferred embodiment the present invention refers to a compound of formula (I), wherein A is A4

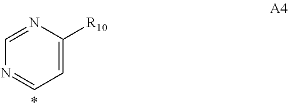

represented by the formula (Id)

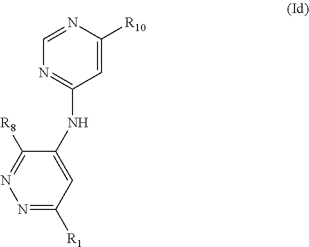

R₁ is aryl optionally substituted by one or more halogen atoms;
R₁₀ is —NR₅C(O)R₆;
R₅ is H;
R₆ is selected from the group consisting of —(C₃-C₆)cycloalkyl substituted by one or more —(C₃-C₉)heterocycloalkyl, wherein said —(C₃-C₉)heterocycloalkyl is optionally substituted by one or more groups selected from —(C₁-C₆)alkyl and —(C₃-C₆)cycloalkyl; —(C₁-C₆)alkylene-(C₃-C₉)heterocycloalkyl, wherein said —(C₃-C₉)heterocycloalkyl is optionally substituted by one or more —(C₁-C₆)alkyl; and —(C₃-C₆)cycloalkyl optionally substituted by one or more —(C₁-C₆)alkylene-(C₃-C₉)heterocycloalkyl, wherein said —(C₃-C₉)heterocycloalkyl is optionally substituted by one or more —(C₁-C₆)alkyl;
R₈ is selected from the gropu consisting of —NR_{A}R_{B}; —S—(C₁-C₆)alkyl, wherein said —(C₁-C₆)alkyl is optionally substituted by one or more —OH; —S—(C₁-C₆)alkylene-OH, wherein said —(C₁-C₆)alkylene is optionally substituted by one or more —(C₁-C₆)alkyl; —S—(C₁-C₆)alkylene-(C₃-C₉)heterocycloalkyl, wherein said —(C₃-C₉)heterocycloalkyl is optionally substituted by one or more —(C₁-C₆)alkyl; —S(O)=NH—(C₁-C₆)alkyl; —S(O)₂—(C₁-C₆)alkyl; —S(O)—(C₁-C₆)alkyl; —S—(C₁-C₆)alkylene-Si((C₁-C₆)alkyl)₃;
R_{A} is H or —(C₁-C₆)alkyl;
R_{B} is selected from the group consisting of —(C₁-C₆)alkylene-(C₃-C₉)heterocycloalkyl, wherein said —(C₃-C₉)heterocycloalkyl is optionally substituted by one or more groups selected from —(C₁-C₆)alkyl and oxo; or alternatively R_{A} and R_{B} together with the nitrogen atom to which they are attached may form a —(C₃-C₆)heterocycloalkyl, wherein said —(C₃-C₆)heterocycloalkyl is optionally substituted by one or more groups selected from —C(O)OH, —(C₁-C₆)alkylene-OH, —C(O)O—(C₁-C₆)alkyl and oxo, or said —(C₃-C₆)heterocycloalkyl is optionally substituted on two adjacent carbon atoms forming an additional condensed —(C₅-C₆)heterocycloalkyl, optionally substituted by oxo; and pharmaceutically acceptable salts thereof.

In a more preferred embodiment the present invention refers to a compound of formula (Id), wherein R₆ is selected from the group consisting of -(4-methylpiperazin-1-yl)cyclobutane, -(4-methylpiperazin-1-yl)ethyl, -(3,5-dimethylpiperazin-1-yl)ethyl, -(4-cyclopropylpiperazin-1-yl)cyclobutene,-[(4-methylpiperazin-1-yl)methyl]bicyclo[1.1.1]pentane, -(3,5-dimethylpiperazin-1-yl)cyclobutane and -(4-methyl-1,4-diazepan-1-yl)methyl; and R₈ is selected from the group consisting of -(2-hydroxyethyl)sulfanyl, N-methyl[(3-methyl-2-oxooxolan-3-yl)methyl]amino and -[2-(trimethylsilyl)ethyl]sulfanyl.

In a more preferred embodiment the present invention refers to a compound of formula (Id), wherein R₆ is selected from the group consisting of -(4-methylpiperazin-1-yl)ethyl and -(3,5-dimethylpiperazin-1-yl)ethyl and R₈ is selected from the group consisting of -(2-hydroxyethyl)sulfanyl and N-methyl[(3-methyl-2-oxooxolan-3-yl)methyl]amino.

According to a preferred embodiment, the invention refers to at least one of the compounds of Formula (Id) listed in the Table 6 below and pharmaceutical acceptable salts thereof. These compounds are particularly active on receptor ALK5, as shown in Table 4.

TABLE 6
List of preferred compounds of Formula (Id)

| Example | | |
|---|---|---|
| No | Structure | Chemical name |
| 91 | 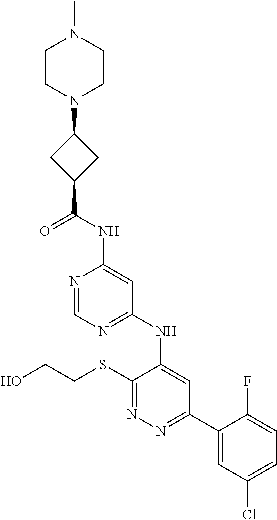 [SMILES: CN1CCN([C@H]2C[C@@H](C(=O)Nc3cc(Nc4cc(-c5cc(Cl)ccc5F)nnc4SCCO)ncn3)C2)CC1] | Cis N-(6-{[6-(5-chloro-2- fluorophenyl)-3-[(2- hydroxyethyl)sulfanyl]pyridazin- 4-yl]amino}pyrimidin-4-yl)- 3-(4-methylpiperazin-1- yl)cyclobutane-1-carboxamide |
| 137 | 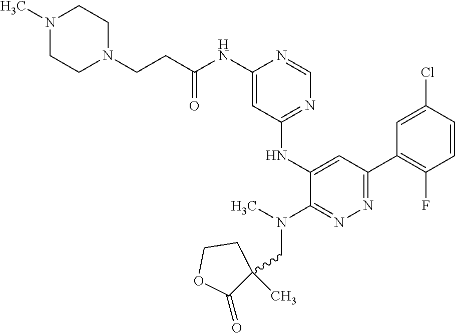 [SMILES: CN1CCN(CCC(=O)Nc2cc(Nc3cc(-c4cc(Cl)ccc4F)nnc3N(C)CC3(C)CCOC3=O)ncn2)CC1] | N-(6-{[6-(5-chloro-2- fluorophenyl)-3-{methyl[(3- methyl-2-oxooxolan-3- yl)methyl]amino}pyridazin-4- yl]amino}pyrimidin-4-yl)-3-(4- methylpiperazin-1- yl)propanamide |
| 138 | 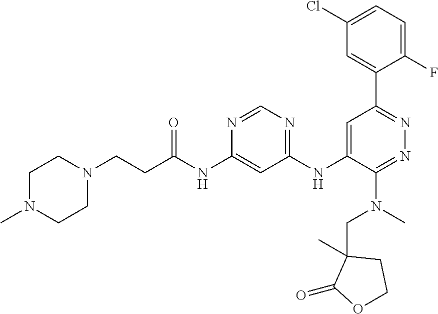 [SMILES: CN1CCN(CCC(=O)Nc2cc(Nc3cc(-c4cc(Cl)ccc4F)nnc3N(C)CC3(C)CCOC3=O)ncn2)CC1] | Enantiomer 1 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3- (4-methylpiperazin-1- yl)propanamide |
| 139 | 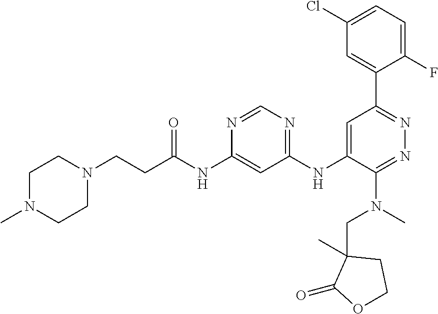 [SMILES: CN1CCN(CCC(=O)Nc2cc(Nc3cc(-c4cc(Cl)ccc4F)nnc3N(C)CC3(C)CCOC3=O)ncn2)CC1] | Enantiomer 2 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3- (4-methylpiperazin-1- yl)propanamide |
| 140 | 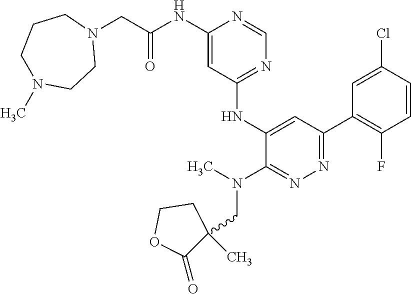 [SMILES: CN1CCCN(CC(=O)Nc2cc(Nc3cc(-c4cc(Cl)ccc4F)nnc3N(C)CC3(C)CCOC3=O)ncn2)CC1] | N-(6-{[6-(5-chloro-2- fluorophenyl)-3-{methyl[(3- methyl-2-oxooxolan-3- yl)methyl]amino}pyridazin-4- yl]amino}pyrimidin-4-yl)-2-(4- methyl-1,4-diazepan-1- yl)acetamide |
| 141 | 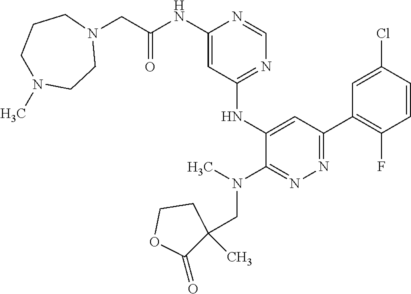 [SMILES: CN1CCCN(CC(=O)Nc2cc(Nc3cc(-c4cc(Cl)ccc4F)nnc3N(C)CC3(C)CCOC3=O)ncn2)CC1] | Enantiomer 1 N-(6-{[6-(5- chloro-2-fluorophenyl)-3- {methyl[(3-methyl-2-oxooxolan- 3-yl)methyl]amino}pyridazin-4- yl]amino}pyrimidin-4-yl)-2-(4- methyl-1,4-diazepan-1- yl)acetamide |
| 142 | 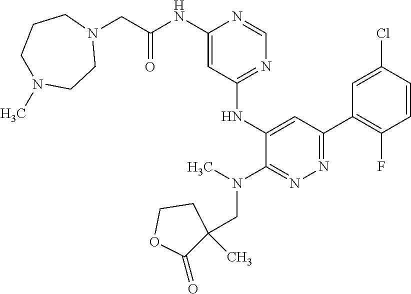 [SMILES: CN1CCCN(CC(=O)Nc2cc(Nc3cc(-c4cc(Cl)ccc4F)nnc3N(C)CC3(C)CCOC3=O)ncn2)CC1] | Enantiomer 2 N-(6-{[6-(5- chloro-2-fluorophenyl)-3- {methyl[(3-methyl-2-oxooxolan- 3-yl)methyl]amino}pyridazin-4- yl]amino}pyrimidin-4-yl)-2-(4- methyl-1,4-diazepan-1- yl)acetamide |
| 143 | 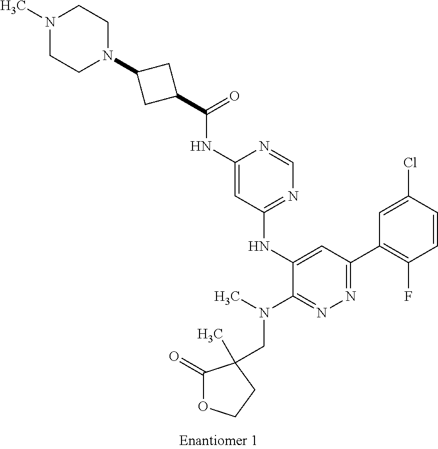 | Cis Enantiomer 1 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3-(4- methylpiperazin-1- yl)cyclobutane-1-carboxamide |
| 144 | 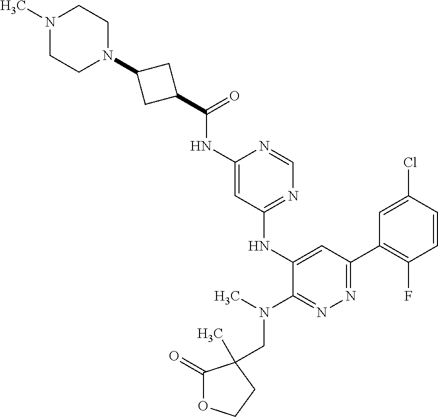 | Cis Enantiomer 2 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3-(4- methylpiperazin-1- yl)cyclobutane-1-carboxamide |
| 176 | 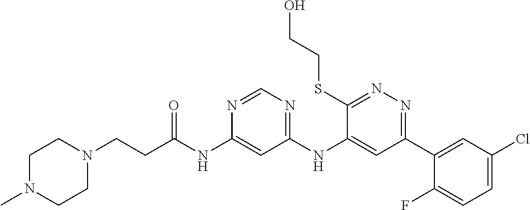 [SMILES: CN1CCN(CCC(=O)Nc2cc(Nc3cc(-c4cc(Cl)ccc4F)nnc3SCCO)ncn2)CC1] | N-(6-{[6-(5-chloro-2- fluorophenyl)-3-[(2- hydroxyethyl)sulfanyl]pyridazin- 4-yl]amino}pyrimidin-4-yl)- 3-(4-methylpiperazin-1- yl)propanamide |
| 177 | 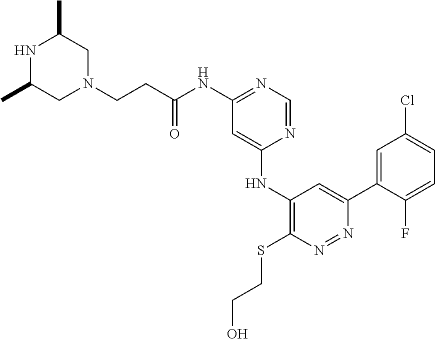 [SMILES: C[C@@H]1CN(CCC(=O)Nc2cc(Nc3cc(-c4cc(Cl)ccc4F)nnc3SCCO)ncn2)C[C@H](C)N1] | N-(6-{[6-(5-chloro-2- fluorophenyl)-3-[(2- hydroxyethyl)sulfanyl]pyridazin- 4-yl]amino}pyrimidin-4-yl)- 3-(3,5-dimethylpiperazin-1- yl)propanamide |
| 181 | 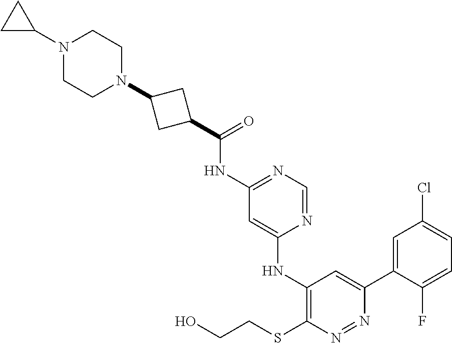 | Cis N-(6-{[6-(5-chloro-2- fluorophenyl)-3-[(2- hydroxyethyl)sulfanyl]pyridazin- 4-yl]amino}pyrimidin-4-yl)- 3-(4-cyclopropylpiperazin-1- yl)cyclobutane-1-carboxamide |
| 182 | 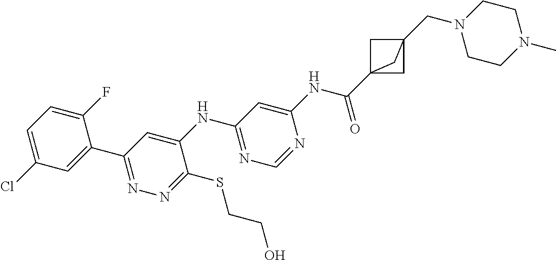 [SMILES: CN1CCN(CC23CC(C(=O)Nc4cc(Nc5cc(-c6cc(Cl)ccc6F)nnc5SCCO)ncn4)(C2)C3)CC1] | N-(6-{[6-(5-chloro-2- fluorophenyl)-3-[(2- hydroxyethyl)sulfanyl]pyridazin- 4-yl]amino}pyrimidin-4-yl)- 3-[(4-methylpiperazin-1- yl)methyl]bicyclo[1.1.1]pentane- 1-carboxamide |
| 185 | 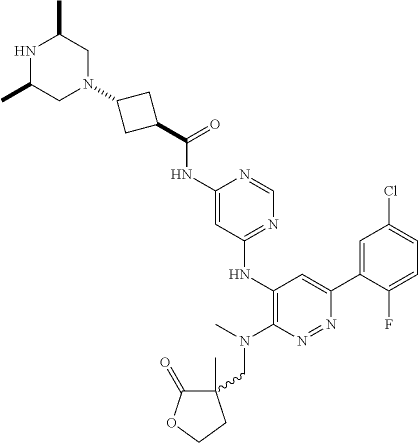 [SMILES: C[C@@H]1CN([C@H]2C[C@H](C(=O)Nc3cc(Nc4cc(-c5cc(Cl)ccc5F)nnc4N(C)CC4(C)CCOC4=O)ncn3)C2)C[C@H](C)N1] | Trans N-(6-{[6-(5-chloro-2- fluorophenyl)-3-{methyl[(3- methyl-2-oxooxolan-3- yl)methyl]amino}pyridazin-4- yl]amino}pyrimidin-4-yl)-3- (3,5-dimethylpiperazin-1- yl)cyclobutane-1-carboxamide |
| 186 | 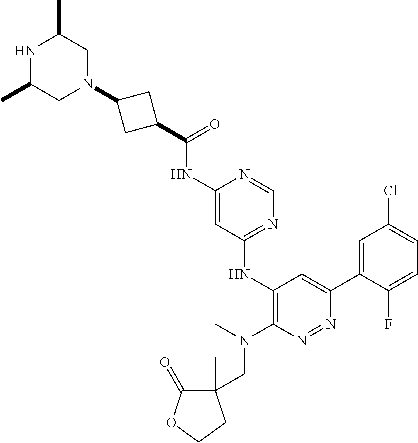 [SMILES: C[C@@H]1CN([C@H]2C[C@@H](C(=O)Nc3cc(Nc4cc(-c5cc(Cl)ccc5F)nnc4N(C)CC4(C)CCOC4=O)ncn3)C2)C[C@H](C)N1] | Cis Enantiomer 1 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3- (3,5-dimethylpiperazin-1- yl)cyclobutane-1-carboxamide |
| 187 | 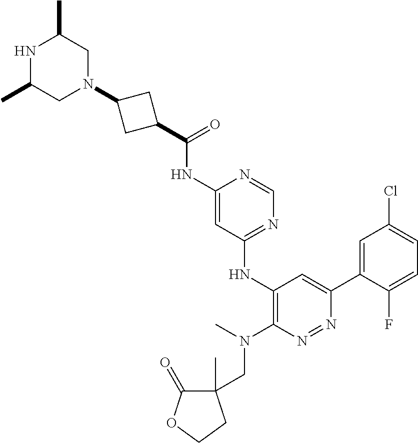 [SMILES: C[C@@H]1CN([C@H]2C[C@@H](C(=O)Nc3cc(Nc4cc(-c5cc(Cl)ccc5F)nnc4N(C)CC4(C)CCOC4=O)ncn3)C2)C[C@H](C)N1] | Cis Enantiomer 2 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3- (3,5-dimethylpiperazin-1- yl)cyclobutane-1-carboxamide |
| 195 | 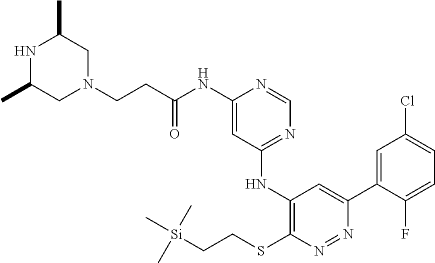 [SMILES: C[C@@H]1CN(CCC(=O)Nc2cc(Nc3cc(-c4cc(Cl)ccc4F)nnc3SCC[Si](C)(C)C)ncn2)C[C@H](C)N1] | N-(6-{[6-(5-chloro-2- fluorophenyl)-3-{[2- (trimethylsilyl)ethyl]sulfanyl}py- ridazin-4-yl]amino}pyrimidin- 4-yl)-3-(3,5-dimethylpiperazin- 1-yl)propanamide |
| 198 | 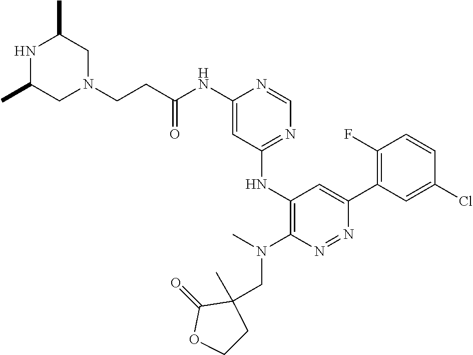 [SMILES: C[C@@H]1CN(CCC(=O)Nc2cc(Nc3cc(-c4cc(Cl)ccc4F)nnc3N(C)CC3(C)CCOC3=O)ncn2)C[C@H](C)N1] | Enantiomer 1 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3- (3,5-dimethylpiperazin-1- yl)propanamide |
| 199 | 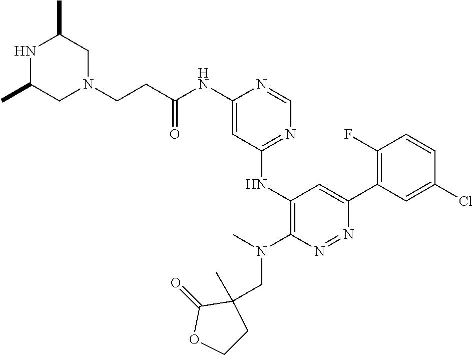 [SMILES: C[C@@H]1CN(CCC(=O)Nc2cc(Nc3cc(-c4cc(Cl)ccc4F)nnc3N(C)CC3(C)CCOC3=O)ncn2)C[C@H](C)N1] | Enantiomer 2 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-3- (3,5-dimethylpiperazin-1- yl)propanamide |
| 208 | 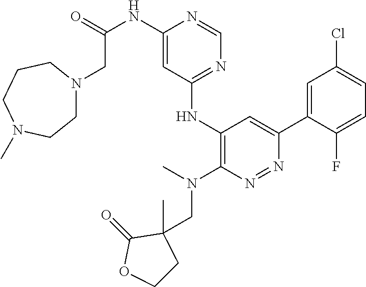 [SMILES: CN1CCCN(CC(=O)Nc2cc(Nc3cc(-c4cc(Cl)ccc4F)nnc3N(C)CC3(C)CCOC3=O)ncn2)CC1] | Enantiomer 1 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-2-(4- methyl-1,4-diazepan-1- yl)acetamide |
| 209 | 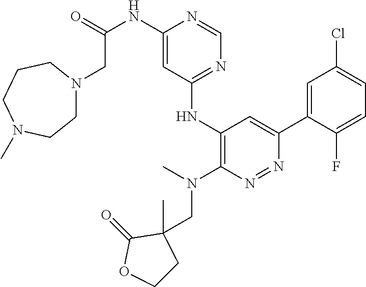 [SMILES: CN1CCCN(CC(=O)Nc2cc(Nc3cc(-c4cc(Cl)ccc4F)nnc3N(C)CC3(C)CCOC3=O)ncn2)CC1] | Enantiomer 2 N-(6-{[6-(5- chloro-2-fluorophenyl)-3- {methyl[(3-methyl-2- oxooxolan-3- yl)methyl]amino}pyridazin-4- yl]amino}pyrimidin-4-yl)-2-(4- methyl-1,4-diazepan-1- yl)acetamide |

The compounds of the invention, including all the compounds here above listed, can be prepared from readily available starting materials using the following general methods and procedures outlined in detail in the Schemes shown below, or by using slightly modified processes readily available to those of ordinary skill in the art. Although a particular embodiment of the present invention may be shown or described herein, those skilled in the art will recognize that all embodiments or aspects of the present invention can be obtained using the methods described herein or by using other known methods, reagents and starting materials. When typical or preferred process conditions (i.e. reaction temperatures, times, mole ratios of reactants, solvents, pressures, etc.) are given, other process conditions can also be used unless otherwise stated. While the optimum reaction conditions may vary depending on the particular reactants or solvent used, such conditions can be readily determined by those skilled in the art by routine optimization procedures. Thus, processes described below should not be viewed as limiting the scope of the synthetic methods available for the preparation of the compounds of the invention.

In some cases a step is needed in order to mask or protect sensitive or reactive moieties, generally known protective groups (PG) could be employed, in accordance to general principles of chemistry (Protective group in organic syntheses, 3rd ed. T. W. Greene, P. G. M. Wuts).

The compounds of formula (I) of the present invention have surprisingly been found to effectively inhibit the receptor ALK5. Advantageously, the inhibition of ALK5 may result in efficacious treatment of the diseases or condition wherein the ALK5 receptor is involved. In this respect, it has now been found that the compounds of formula (I) of the present invention have an inhibitory drug potency, expressed as pIC₅₀ (negative logarithm of IC₅₀, half maximal inhibitory concentration) and subsequently converted to pKᵢ (negative logarithm of dissociate function Kᵢ), equal or higher than 8.5 on ALK5, as shown in the experimental part. Preferably, the compounds of the present invention have a pKᵢ on ALK5 between 8.5 and 9.4, more preferably between 9.5 and 9.9 and even more preferably higher or equal than 10.

In one aspect, the present invention refers to a compound of formula (I) or a pharmaceutically acceptable salt thereof, for use as a medicament. Thus, the invention refers to a compound of formula (I) in the preparation of a medicament, preferably for use in the prevention and/or treatment of a disease, disorder or condition associated with ALK5 signaling pathway.

In a preferred embodiment, the invention refers to a compound of formula (I) or a pharmaceutically acceptable salt thereof, for use in the prevention and/or treatment of a disease, disorder or condition associated with ALK5 signaling pathway.

In one embodiment, the present invention refers to a compound of formula (I) useful for the prevention and/or treatment of fibrosis and/or diseases, disorders, or conditions that involve fibrosis.

The terms “fibrosis” or “fibrosing disorder,” as used herein, refers to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract.

Preferably, the compounds of formula (I) of the present invention, or a pharmaceutical composition comprising a compound of formula (I), are useful for the treatment and/or prevention of fibrosis such as pulmonary fibrosis, idiopathic pulmonary fibrosis (IPF), hepatic fibrosis, renal fibrosis, ocular fibrosis, cardiac fibrosis, arterial fibrosis and systemic sclerosis.

More preferably, the compounds of formula (I) of the present invention, or a pharmaceutical composition comprising a compound of formula (I), are useful for the treatment of idiopathic pulmonary fibrosis (IPF).

As used herein, “safe and effective amount” in reference to a compound of formula (I) or a pharmaceutically acceptable salt thereof or other pharmaceutically-active agent means an amount of the compound sufficient to treat the patient's condition but low enough to avoid serious side effects and it can nevertheless be routinely determined by the skilled artisan.

The compounds of formula (I) may be administered once or according to a dosing regimen wherein a number of doses are administered at varying intervals of time for a given period of time. Typical daily dosages may vary depending upon the route of administration chosen.

The present invention also refers to a pharmaceutical composition comprising a compound of formula (I) in admixture with at least one or more pharmaceutically acceptable carrier or excipient.

In one embodiment, the invention refers to a pharmaceutical composition of compounds of formula (I) in admixture with one or more pharmaceutically acceptable carrier or excipient, for example those described in Remington's Pharmaceutical Sciences Handbook, XVII Ed., Mack Pub., N.Y., U.S.A.

Administration of the compounds of the invention and their pharmaceutical compositions may be accomplished according to patient needs, for example, orally, nasally, parenterally (subcutaneously, intravenously, intramuscularly, intrasternally and by infusion) and by inhalation. Preferably, the compounds of the present invention are administered orally or by inhalation. More preferably, the compounds of the present invention are administered by inhalation.

In one preferred embodiment, the pharmaceutical composition comprising the compound of formula (I) is a solid oral dosage form such as tablets, gelcaps, capsules, caplets, granules, lozenges and bulk powders.

In one embodiment, the pharmaceutical composition comprising the compound of formula (I) is a tablet.

The compounds of the invention can be administered alone or combined with various pharmaceutically acceptable carriers, diluents (such as sucrose, mannitol, lactose, starches) and known excipients, including suspending agents, solubilizers, buffering agents, binders, disintegrants, preservatives, colorants, flavorants, lubricants and the like.

In a further embodiment, the pharmaceutical composition comprising a compound of formula (I) is a liquid oral dosage forms such as aqueous and non-aqueous solutions, emulsions and suspensions. Such liquid dosage forms can also contain suitable known inert diluents such as water and suitable known excipients such as preservatives, wetting agents, sweeteners, flavorants, as well as agents for emulsifying and/or suspending the compounds of the invention.

In a further embodiment, the pharmaceutical composition comprising the compound of formula (I) is an inhalable preparation such as inhalable powders, propellant-containing metering aerosols or propellant-free inhalable formulations.

For administration as a dry powder, single- or multi-dose inhalers known from the prior art may be utilized. In that case the powder may be filled in gelatine, plastic or other capsules, cartridges or blister packs or in a reservoir.

A diluent or carrier chemically inert to the compounds of the invention, e.g. lactose or any other additive suitable for improving the respirable fraction may be added to the powdered compounds of the invention.

Inhalation aerosols containing propellant gas such as hydrofluoroalkanes may contain the compounds of the invention either in solution or in dispersed form. The propellant-driven formulations may also contain other ingredients such as co-solvents, stabilizers and optionally other excipients.

The propellant-free inhalable formulations comprising the compounds of the invention may be in form of solutions or suspensions in an aqueous, alcoholic or hydroalcoholic medium and they may be delivered by jet or ultrasonic nebulizers known from the prior art or by soft-mist nebulizers.

The compounds of the invention can be administered as the sole active agent or in combination with other pharmaceutical active ingredients.

The dosages of the compounds of the invention depend upon a variety of factors including among others the particular disease to be treated, the severity of the symptoms, the route of administration and the like.

The invention is also directed to a device comprising a pharmaceutical composition comprising a compound of formula (I) according to the invention, in form of a single- or multi-dose dry powder inhaler or a metered dose inhaler.

All preferred groups or embodiments described above for compounds of formula (I) may be combined among each other and apply as well mutatis mutandis.

In a first embodiment of the present invention, compounds of formula (I) can be prepared according to the following synthetic routes described in Scheme 1.

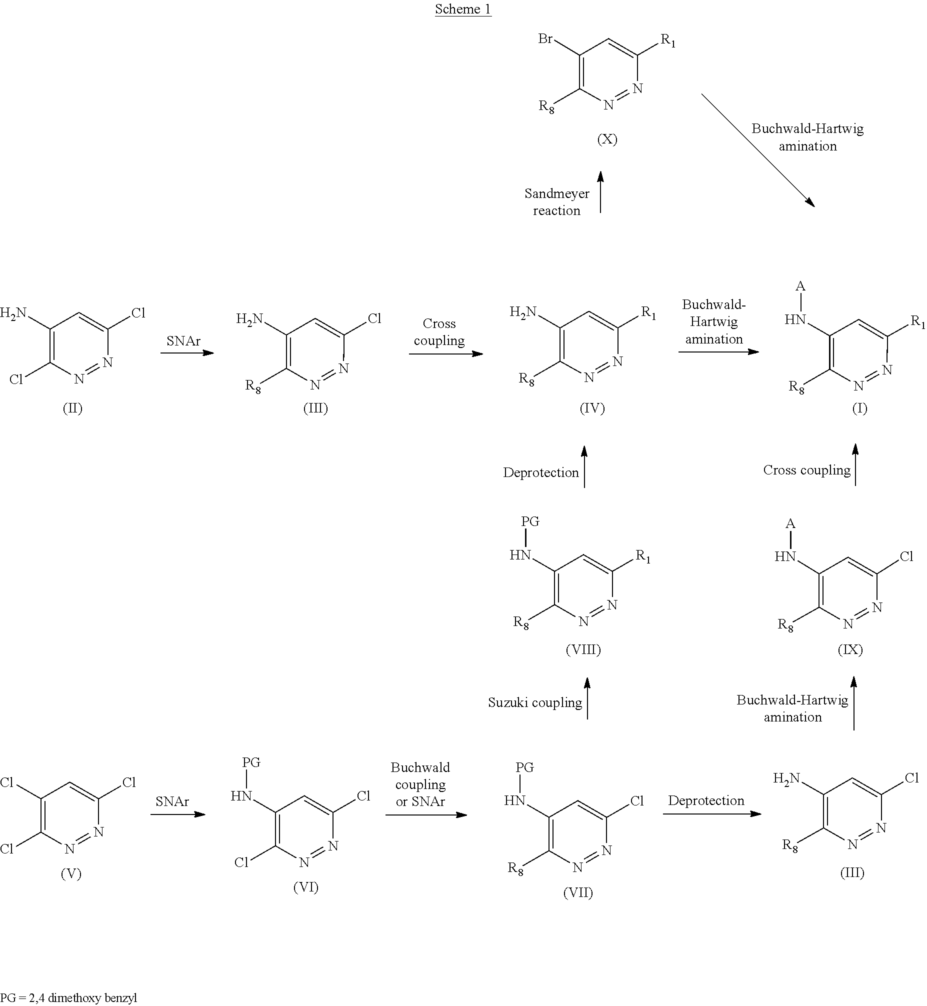

Compounds of formula (III) may be obtained by reacting commercially available compound (II) with appropriate alcohol, amine or thiol under nucleophilic aromatic substitution (SNAr). Typical reaction conditions comprise a suitable base, such as NaH or K₂CO₃, a proper solvent as DMF or THF, and an appropriate temperature, usually between room temperature and 130° C. Reaction of compounds (III) under metal-catalyzed cross coupling conditions afforded compounds (IV). Typical cross-coupling reaction may be Suzuki coupling, or similar as described in “Transition Metals for 15 Organic Synthesis”, 2nd Ed, 1, 2004. Representative Suzuki reaction conditions include reacting compound (III) with a suitable boronic acid, in the presence of base, such as K₂CO₃ and Pd catalyst, as PdCl₂(PPh₃)₂·DCM, in a mixture of solvents, such as 1,4 dioxane and water, at an appropriate temperature, such as, for example, 100° C. Finally, a compound of formula (I) may be obtained by reacting a compound of formula (IV) with a suitable halide under standard Buchwald-Hartwig amination conditions. Typical Buchwald-Hartwig conditions involve the presence of an appropriate base, such as Cs₂CO₃, a suitable ligand reagent, such as Xantphos, and a suitable catalyst such as Pd(OAc)₂, in an appropriate solvent as, for example, 1,4-dioxane and at an appropriate temperature, such as, for example, 100° C. Alternatively, compounds of formula (I) can be obtained starting from commercially available compound (V). In this case, SNAr of compound (V) with 2,4-dimethoxybenzylamine in a suitable solvent, such as THF, typically at 50° C., may lead to compound (VI). Introduction of R₈ to afford compound of formula (VII) may be achieved using, for example, metal-catalyzed cross coupling reaction such as Buchwald-Hartwig amination with the suitable amine, or by SNAr with the proper nucleophile. Representative Buchwald-Hartwig amination conditions involve the use of an appropriate base, such as Cs₂CO₃, palladium catalyst, as Pd₂(dba)₃, and a suitable ligand such as tBuXPhos. Such reactions are usually carried on in appropriate solvents, as toluene, and at appropriate temperatures, such as, for example, 90° C. Typical SNAr conditions include an appropriate base such as NaH in a suitable solvent such as DMF, and at an appropriate temperature, such as, for example, 130° C. Reaction of compound of formula (VII) with the suitable boronic acid under Suzuki cross coupling conditions, as described above, can lead to compounds (VIII). Removal of the 2,4-dimethoxybenzyl protecting group under acidic conditions, such as, for example, TFA solution in DCM at room temperature, allowed to obtain compounds of formula (IV), which may react with proper halides under the previously described Buchwald-Hartwig amination conditions to afford compounds of formula (I). Alternatively, compound of formula (IV) may react under Sandmeyer conditions to afford compound (X). Representative Sandmeyer reaction conditions involve the presence of tert-butyl nitrite, an appropriate catalytic copper salt, such as copper (II) bromide, an appropriate solvent such as MeCN and a suitable temperature, such as, for example, 25° C. Finally, insertion of group A on compound of formula (X) may be achieved by reaction with a suitable amine under standard Buchwald-Hartwig amination conditions to obtain compound of formula (I). In this case, typical Buchwald-Hartwig conditions involve the presence of an appropriate base, such as K₃PO₄, a suitable ligand reagent, such as Xantphos, and a suitable catalyst such as Pd₂(dba)₃, in an appropriate solvent such as 1,2-dimethoxyethane and at an appropriate temperature, such as, for example, 110° C. In some cases, compounds of formula (VII) can first undergo deprotection under acidic conditions as described above, to yield compounds (III). In these cases, compounds (III) can then be reacted with suitable halides under Buchwald-Hartwig amination conditions to give compounds (IX). Typical Buchwald-Hartwig conditions involve the presence of an appropriate base, such as cesium carbonate, a suitable ligand reagent, such as Xantphos, and a suitable catalyst such as Pd(OAc)₂, in an appropriate solvent such as 1,4-dioxane and at an appropriate temperature, such as, for example, 100° C. Compounds (IX) can partecipate to metal-catalyzed cross coupling reaction to introduce the proper R₁ group. Cross-coupling reactions may be Suzuki or Stille coupling. Representative Suzuki reaction conditions are those described above, while typical Stille coupling conditions involve the presence of a suitable stannane, and a suitable catalyst such as Pd(dppf)Cl₂, in an appropriate solvent such as DMF and at an appropriate temperature, such as, for example, 100° C.

In another embodiment, compounds of formula (I) can be prepared as described in Scheme 2.

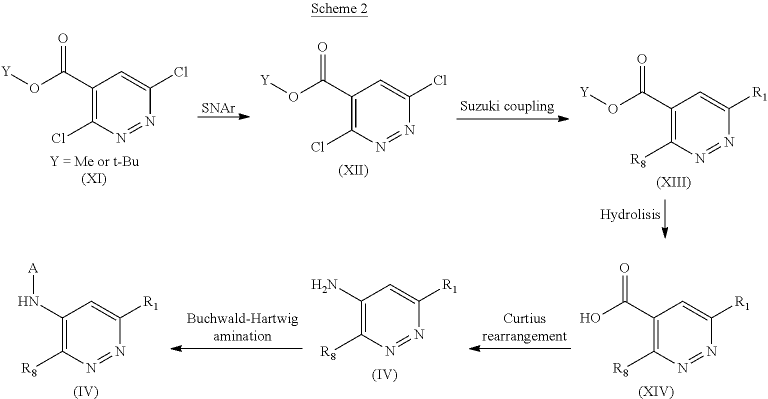

Compounds of formula (XII) may be obtained from commercially available compound (XI) by SNAr with appropriate amine in a suitable solvent, such as 1,2-dimethoxyethane, in presence of a suitable base such as DIPEA, at an appropriate temperature, such as between 8° and 110° C. Introduction of R₁ to afford compounds of formula (XIII) may be achieved reacting compound (XII) in a metal-catalyzed cross coupling reaction, such as a Suzuki coupling, under the reaction conditions described above. Ester hydrolysis of compound (XIII) under acidic or basic conditions, well known to the person skilled in the art, afforded the corresponding carboxylic acid (XIV), which may undergo Curtius rearrangement in the presence of diphenylphosphoryl azide (DPPA), a suitable base, such as triethylamine, and in a proper solvent such as t-BuOH, at an appropriate temperature, such as 90° C., to yield compounds of formula (IV). Finally, reaction of compound (IV) under standard Buchwald-Hartwig amination conditions described above can afford compounds of formula (I).

In another embodiment, compounds of formula (I) can be prepared as described in Scheme 3.

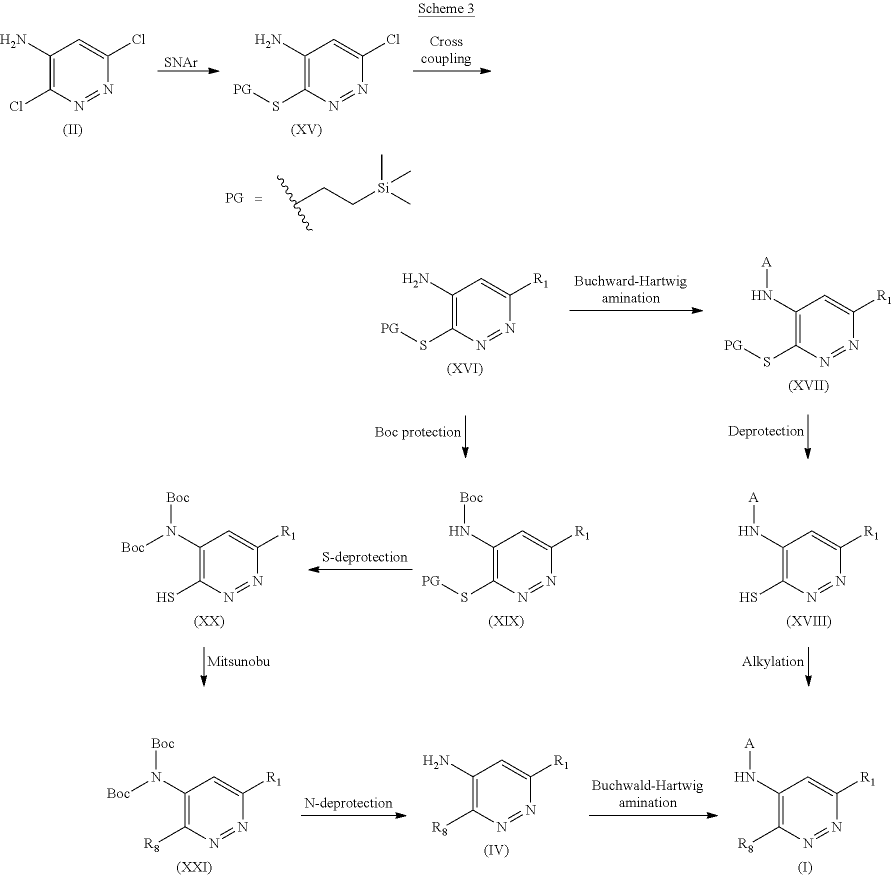

Compound of formula (XV) may be obtained from commercially available compound (II) by SNAr with an appropriate protected thiol, in a suitable solvent such as DMF, in the presence of a suitable base such as NaH, at an appropriate temperature, such as between 0 and 25° C. Introduction of R1 to afford a compound of formula (XVI) may be achieved reacting compounds (XV) in a metal-catalyzed cross coupling reaction, such as Suzuki coupling, under reaction conditions described above.

A compound of formula (XVI) can react with proper halide under Buchwald-Hartwig amination, according to the conditions well described above, to afford a compound of formula (XVII). Thiol deprotection following standard literature conditions, such as the use of tetrabutyl ammonium fluoride (TBAF) in a suitable solvent, as THF, and at an appropriate temperature, such as room temperature, may lead to compounds (XVIII). In this case, final introduction of R₈ to afford compounds of formula (I) may be achieved by alkylation of compounds (XVIII) with an appropriate alkylating agent, with or without a suitable base, such as for example Na₂CO₃, in a suitable solvent as DMF, and at an appropriate temperature, such as between 25 and 60° C. Alternatively, compounds of formula (XVI) can first be converted in compounds (XIX) by reaction with di-tert-butyl dicarbonate (Boc anhydride, Boc₂O) in the presence of a base, such as triethylamine, in a suitable solvent such as DCM, at an appropriate temperature, such as, for example, 25° C. Compounds of formula (XX) can be achieved by S-deprotection of compounds (XIX) under standard literature conditions, as previously described, and can react under Mitsunobu reaction conditions with suitable alcohols to afford compounds of formula (XXI). Representative Mitsunobu conditions include the use of triphenylphosphine, the appropriate azodicarboxylate reagent, such as diisopropyl azodicarboxylate (DIAD), in a proper polar aprotic solvent, as THF, and at the suitable temperature, as for example 55° C. N-deprotection of compound (XXI) under acidic conditions, such as, for example, TFA solution in DCM at room temperature, allowed to obtain compounds of formula (IV). Lastly, reaction of compound (IV) under standard Buchwald-Hartwig amination conditions, well described above, may lead to compounds of formula (I).

In a further embodiment, compounds of formula (I) can be prepared as described in Scheme 4.

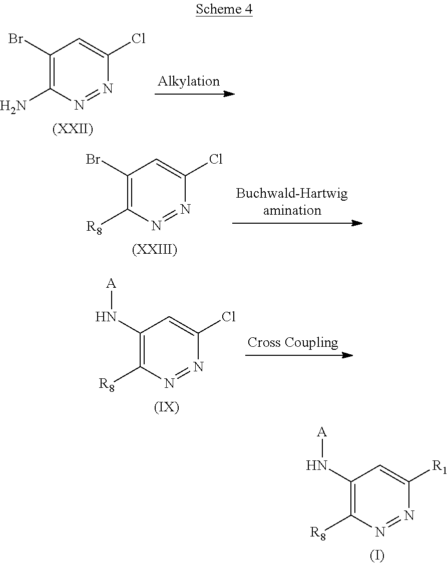

Compound of formula (XXIII) may be obtained from commercially available compound (XXII) by alkylation with an appropriate alkylating agent, in the presence of suitable base, such as NaH, in a suitable solvent such as THF, and at an appropriate temperature, such as between 0 and 40° C. Compounds (XXIII) can undergo Buchwald-Hartiwg amination in the presence of suitable amines to yield compounds (IX). Typical Buchwald-Hartwig conditions comprise a proper base, such as K₃PO₄, a suitable ligand reagent, such as Xantphos, and a suitable catalyst such as Pd₂(dba)₃, in an appropriate solvent such as 1,4-dioxane and at an appropriate temperature, such as, for example, 120° C. Lastly, compounds of formula (I) can be obtained from compounds (IX) as described in Scheme 1.

In another embodiment, compounds of formula (I), wherein R₈ is selected from the group consisting of —S(O)=NH—(C₁-C₆)alkyl, —S(O)₂—(C₁-C₆)alkyl and —S(O)—(C₁-C₆)alkyl, can be prepared as described in Scheme 5.

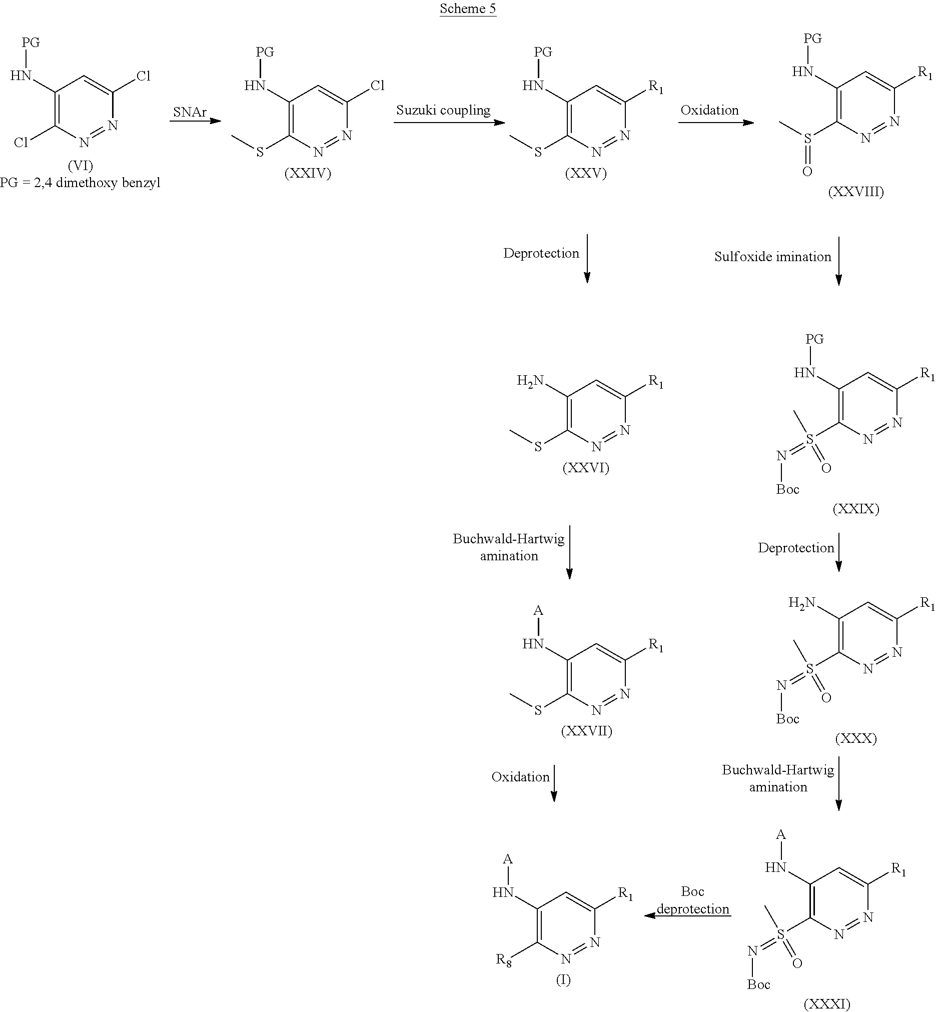

Compounds (VI) can undergo SNAr reaction in the presence of sodium methanethiolate in a suitable solvent, such as DMF, and typically at 25° C., to yield compounds (XXIV), which can react with the proper boronic acid under Suzuki cross-coupling reaction, to give compounds of formula (XXV). Typical Suzuki reaction conditions are well described in the previous schemes. In the cases when R₈ is selected from the group consisting of —S(O)₂—(C₁-C₆)alkyl and —S(O)—(C₁-C₆)alkyl, compounds (XXV) can be first deprotected under acidic conditions, such as, for example, with TFA solution in DCM at room temperature, to give compounds of formula (XXVI). Buchwald-Hartwig amination in the presence of suitable halides may lead to compounds (XXVII). Typical Buchwald-Hartwig conditions involve the presence of an appropriate base, such as Cs₂CO₃, a suitable ligand reagent, such as Xantphos, and a suitable catalyst such as Pd(OAc)₂, in an appropriate solvent as 1,4-dioxane and at an appropriate temperature, such as, for example, 100° C. Oxidation of compounds (XXVII) with an appropriate oxidizing agent such as Oxone®, in a mixture of solvents, such as methanol and water, and at an appropriate temperature, such as, for example, 25° C., afforded compounds of formula (I), wherein R8 is selected from the group consisting of —S(O)₂—(C₁-C₆)alkyl and —S(O)—(C₁-C₆)alkyl. In the cases when R₈ is —S(O)=NH—(C₁-C₆)alkyl, a compound of formula (XXV) may be first oxidized with an appropriate oxidizing agent such as Oxone®, under the reaction conditions described above, to afford compound (XXVIII). Compound (XXIX) may be obtained by sulfoxide imination of compound (XXVIII). Prototypical reaction conditions involve a proper source of nitrogen, such as 1,3-bis(1,1-dimethylethyl) imidodicarbonate, ammonium acetate and the like, a suitable catalyst, such as rhodium(II) acetate dimer in combination with magnesium oxide and iodobenzene diacetate, in a suitable solvent such as DCM, and at an appropriate temperature, as, for example, 40° C. Removal of 2,4-dimethoxybenzyl protecting group from compounds (XXIX) to afford compounds (XXX) may be achieved under standard literature conditions such as by reaction with ammonium cerium(IV) nitrate (CAN) in a suitable mixture of solvents, such as MeCN and water, at room temperature. Insertion of group A on a compound of formula (XXX) may be achieved by reaction with a suitable halide under standard Buchwald-Hartwig amination conditions described above to obtain compound of formula (XXXI). Finally, removal of the Boc protecting group under acidic conditions, such as, for example, TFA solution in DCM at room temperature, allowed to obtain compounds of formula (I), wherein R₈ is —S(O)=NH—(C₁-C₆)alkyl.

The various aspects of the invention described in this application are illustrated by the following examples which are not meant to limit the invention in any way.

### Preparations of Intermediates and Examples

Chemical Names of the compounds were generated with Structure To Name Enterprise 10.0 Cambridge Software. All reagents, for which the synthesis is not described in the experimental part, are either commercially available, or are known compounds or may be formed from known compounds by known methods by a person skilled in the art.

### Abbreviation—Meaning

Boc=tert-Butyloxycarbonyl; c-Hex=Cyclohexane; Cs₂CO₃=Cesium carbonate; DCM=Dichloromethane; de=Diastereomeric excess; DIPEA=N,N-Diisopropylethylamine; DMAP=4-(Dimethylamino)pyridine; DMF=Dimethylformamide; DMSO=Dimethylsulfoxide; ee=Enantiomeric excess; EtOAc=Ethyl acetate; HATU=1-[Bis(dimethylamino)methylene]-1H-1,2,3-triazolo[4,5-b]pyridinium 3-oxid hexafluorophosphate; HCOOH=Formic acid; h=hour; hrs=hours; HCl=Hydrochloric acid; H₂=Hydrogen; H₂O=Water; Int=intermediate; K₂CO₃=Potassium carbonate; K₃PO₄=Potassium phosphate tribasic; KF=potassium fluoride; LC-MS=liquid chromatography/mass spectrometry; MeCN=Acetonitrile; MeOH=Methanol; N₂=Nitrogen; NaH=sodium hydride; Na₂SO₄=Sodium sulfate; NaHCO₃=Sodium bicarbonate; Na₂CO₃=Sodium carbonate; Na₂S₂O₈=Sodium persulfate; NH₃=Ammonia; NH₄Cl=ammonium chloride; NH₄OH=ammonium hydroxide; NMP=1-Methyl-2-pyrrolidone; MW=Microwaves; PdCl₂(PPh₃)₂=Bis(triphenylphosphine)palladium(II) dichloride; Pd₂(dba)₃=Tris(dibenzylideneacetone)dipalladium(0); Pd(dppf)Cl₂=[1,1′-Bis(diphenylphosphino)ferrocene]dichloropalladium(II); Pd(dppf)Cl₂ DCM=[1,1′-Bis(diphenylphosphino)ferrocene]dichloropalladium(II), complex with dichloromethane; Pd(OAc)₂=Palladium(II) acetate; Pd(PPh₃)₄-Tetrakis(triphenylphosphine)palladium(0); PL-HCO₃=polymer supported hydrogencarbonate; PPh₃=triphenylphosphine; RT=room temperature; SCX=Strong Cation Exchange; tBuXPhos=ditert-butyl-[2-[2,4,6-tri(propan-2-yl)phenyl]phenyl]phosphine; TEA=triethylamine; TFA=trifluoroacetic acid; THF=Tetrahydrofuran; Xantphos=4,5-Bis(diphenylphosphino)-9,9-dimethylxanthene.

### General Experimental Details and Methods

### Analytical Method

### Instruments, Materials and Methods Employed for Analysis

¹H-NMR spectra were performed on a Varian MR-400 spectrometer operating at 400 MHZ (proton frequency), equipped with: a self-shielded Z-gradient coil 5 mm 1H/nX broadband probe head for reverse detection, deuterium digital lock channel unit, quadrature digital detection unit with transmitter offset frequency shift, or on Agilent VNMRS-500, or on a Bruker Avance 400, or on a Agilent Inova 600 operating at 600 MHz equipped with 5 mm PFG PENTA Probe spectrometers. Chemical shifts are reported as 6 values in ppm relative to trimethylsilane (TMS) as an internal standard. Coupling constants (J values) are given in hertz (Hz) and multiplicities are reported using the following abbreviation (s=singlet, d=doublet, t=triplet, q=quartet, m=multiplet, br. s=broad singlet, br. d=broad doublet, br. t=broad triplet, br. dd=broad doublet-doublet, nd=not determined, dd=double-doublet, dt=doublet of triplets, ddd=double-double-doublet, dddd=doublet of doublet of doublet of doublets, quin=quintuplet, td=triple doublet, tt=triple triplet, dq=doublet of quartets, spt=septet).

### LC/UV/MS Analytical Methods

LC/MS retention times are estimated to be affected by an experimental error of +0.5 min. LCMS may be recorded under the following conditions: diode array DAD chromatographic traces, mass chromatograms and mass spectra may be taken on UPLC/PDA/MS Acquity™ system coupled with Micromass ZQTM or Waters SQD single quadrupole mass spectrometer operated in positive and/or negative electron spray ES ionization mode and/or Fractionlynx system used in analytical mode coupled with ZQTM single quadrupole operated in positive and/or negative ES ionisation mode. Quality Control methods used operated under low pH conditions or under high pH conditions:
Method 1, low pH conditions column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 1 mL/min. The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.

Method 2, high pH conditions: column: Acquity Kinetex 1.7 um EVO C18 100A, 2.1×50 mm, the column temperature was 40° C.; mobile phase solvent A was 10 mM aqueous solution of NH₄HCO₃ adjusted to pH=10 with ammonia, mobile phase solvent B MeCN. The flow rate was 1 mL/min. The gradient table was t=0 min 97% A 3% B, t=1.5 min 0.1% A 99.9% B, t=1.9 min 0.1% A 99.9% B and t=2 min 97% A 3% B. The UV detection range was 210-350 nm and ES+/ES− range was 100 to 1500 AMU.
Method 3, low pH conditions column: Acquity CSH C18 2.1×50 mm 1.7 um, the column temperature was 40° C.; mobile phase solvent A was milliQ water+0.1% HCOOH, mobile phase solvent B MeCN+0.1% HCOOH. The flow rate was 0.9 mL/min. The gradient table was t=0 min 97% A 3% B, t=1.4 min 0.1% A 99.9% B, t=1.9 min 0.1% A 99.9% B and t=2 min 97% A 3% B. The UV detection range was 210-350 nm and ES+/ES− range was 100 to 1000 AMU.
Method 4, high pH conditions: column: Acquity Kinetex 1.7 um EVO C18 100A, 2.1×50 mm, the column temperature was 40° C.; mobile phase solvent A was 10 mM aqueous solution of NH₄HCO₃ adjusted to pH=10 with ammonia, mobile phase solvent B MeCN. The flow rate was 0.9 mL/min. The gradient table was t=0 min 97% A 3% B, t=1.4 min 0.1% A 99.9% B, t=1.9 min 0.1% A 99.9% B and t=2 min 97% A 3% B. The UV detection range was 210-350 nm and ES+/ES− range was 100 to 1000 AMU.

### Preparations of Intermediates

### Intermediate 1: N-(4-bromopyridin-2-yl)prop-2-enamide

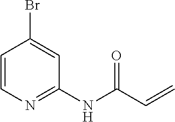

A mixture of 4-bromo-2-pyridinamine (3.0 g, 17.3 mmol) and TEA (7.25 mL, 52.0 mmol) in dry DCM (80 mL) was stirred under N₂ at 0° C., then a solution of 3-chloropropanoyl chloride (1.83 mL, 19.1 mmol) in DCM (20 mL) was added dropwise. The resulting mixture was stirred at 0° C. for 1 h. Water was added and the organic solution was separated and washed with brine, dried over Na₂SO₄ and filtered. The solvent was evaporated, the product was purified by flash chromatography on Biotage silica cartridge (from c-Hex to 25% EtOAc) to afford the title compound (2.4 g, 10.6 mmol, 61% yield).

LC-MS (ESI): m/z (M+1): 226.9 (Method 1)

### Intermediate 2: N-(4-bromopyridin-2-yl)-3-(4-methylpiperazin-1-yl)propanamide

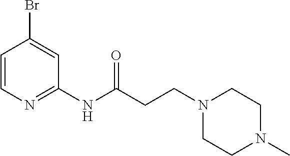 [SMILES: CN1CCN(CCC(=O)Nc2cc(Br)ccn2)CC1]

Intermediate 1 (1.8 g, 6.90 mmol) was dissolved in THE (8 mL), 1-methylpiperazine (1.15 mL, 10.4 mmol) was added and the reaction was stirred at 65° C. for 3 hrs. Volatiles were removed under vacuum and the residue was purified by flash chromatography on Biotage silica NH cartridge (from c-Hex to 50% EtOAc) to afford the title compound (2.4 g, recovery assumed quantitative).

LC-MS (ESI): m/z (M+1): 327.2 (Method 1)

### Intermediate 3: 2-[(4-amino-6-chloropyridazin-3-yl)oxy]ethan-1-ol

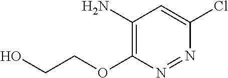 [SMILES: Nc1cc(Cl)nnc1OCCO]

NaH (60% dispersion in oil) (268 mg, 6.71 mmol) was added portion wise to ethane-1,2-diol (4.0 mL, 71.5 mmol) stirred under N₂ at RT. After 30 min 3,6-dichloropyridazin-4-amine (1.0 g, 6.1 mmol) was added. The reaction was heated at 100° C. for 1 h. After cooling down the mixture was treated with cold water and pH was adjusted at 7-8 using 1N HCl. The solid obtained was filtered, washed with water and c-Hex, then collected, and dried to afford the title compound (900 mg, 4.75 mmol, 78% yield). LC-MS (ESI): m/z (M+1): 190.2 (Method 2).

### Intermediate 4: 2-{[4-amino-6-(5-chloro-2-fluorophenyl)pyridazin-3-yl]oxy}ethan-1-ol

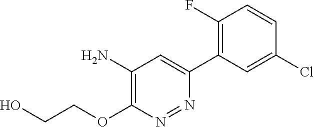 [SMILES: Nc1cc(-c2cc(Cl)ccc2F)nnc1OCCO]

A mixture of 5-chloro-2-fluorobenzeneboronic acid (825 mg, 4.73 mmol), KF (537 mg, 9.1 mmol) and Intermediate 3 (750 mg, 3.64 mmol) in MeCN (10 mL) and H₂O (2 mL) was degassed with N₂ for 2 min, then PdCl₂(PPh₃)₂ (256 mg, 0.36 mmol) was added and the mixture was irradiated with microwaves at 110° C. for 1 h and 15 min. After cooling down the solvents were removed by reduced pressure. The residue was treated with EtOAc/MeOH and filtered on Celite® pad. Organic solvents were evaporated and the residue was purified by flash chromatography on Biotage silica cartridge (100% EtOAc) to afford the title compound (350 mg, 1.23 mmol, 34% yield).

LC-MS (ESI): m/z (M+1): 284.0 (Method 2)

### Intermediate 5: 4-chloro-7-[2-(4-methylpiperazin-1-yl)ethoxy]quinoline

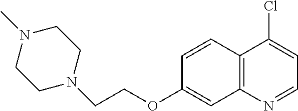 [SMILES: CN1CCN(CCOc2ccc3c(Cl)ccnc3c2)CC1]

4-chloro-7-hydroxyquinoline (200 mg, 1.11 mmol) was added to a stirred mixture of PPh₃ (380 mg, 1.45 mmol) and 2-(4-methylpiperazin-1-yl)ethanol (177 mg, 1.22 mmol) in a mixture of THF (6.67 mL) and NMP (0.67 mL) at RT under N₂. Then diisopropyl azodicarboxylate (0.23 mL, 1.17 mmol) was added dropwise, the resulting mixture was stirred at RT 3 hrs. The mixture was poured in water and extracted with EtOAc. Organic layer was separated, dried over Na₂SO₄, filtered, and evaporated under vacuum. The residue was diluted with water and acidified using 1N HCl under stirring. Aqueous phase was washed with Et₂O and organic layer was discarded. Then aqueous phase was treated with 33% aqueous NH₄OH until pH 9-10 and extracted with DCM. The combined organic layers were dried over Na₂SO₄, filtered and evaporated to afford the title compound (290 mg, 0.95 mmol, 85% yield).

LC-MS (ESI): m/z (M+1): 306.1 (Method 1)

### Intermediate 6: 3,6-dichloro-N-[(2,4-dimethoxyphenyl)methyl]pyridazin-4-amine

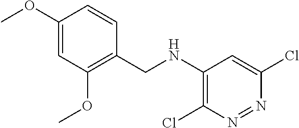 [SMILES: COc1ccc(CNc2cc(Cl)nnc2Cl)c(OC)c1]

To a solution of 3,4,6-trichloropyridazine (5 g, 27.3 mmol) in dry THE (54.5 mL), 1-(2,4-dimethoxyphenyl)methanamine (12.3 mL, 81.8 mmol) was added. The mixture was heated at 50° C. for 15 min. Volatiles were removed under vacuum. The residue was taken-up with EtOAc, washed with water and brine. The organic phase was filtered through a phase separator and evaporated under vacuum. The crude material was purified by flash chromatography on Biotage silica NH cartridge (from 0% to 40% of EtOAc % in c-Hex). After evaporation, a solid precipitated, it was triturated with DCM and EtOAc to afford a first crop. The filtrate was evaporated and purified again by flash chromatography on Biotage silica cartridge (from 0% to 5% of MeOH in DCM). The product so obtained was mixed with the first batch to afford the title compound (8.33 g, 26.5 mmol, 97% yield). LC-MS (ESI): m/z (M+1): 314.1 (Method 2)

### Intermediate 7: 6-chloro-3-(2,2-difluoroethoxy)-N-[(2,4-dimethoxyphenyl)methyl]pyridazin-4-amine

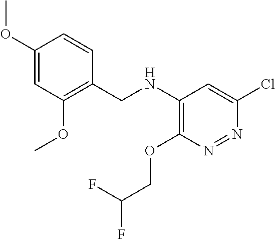 [SMILES: COc1ccc(CNc2cc(Cl)nnc2OCC(F)F)c(OC)c1]

A mixture of Intermediate 6 (550 mg, 1.75 mmol), tBuXPhos (89 mg, 0.21 mmol), Pd₂(dba)₃ (96 mg, 0.11 mmol), Cs₂CO₃ (1.72 g, 5.25 mmol) was suspended in toluene (11 mL). The mixture was degassed (vacuum/N₂) and 2,2-difluoroethanol (144 μL, 2.28 mmol) was added by syringe and the mixture was heated at 90° C. overnight. The mixture was diluted with EtOAc, and filtered through a pad of Celite®, washing the cake with EtOAc. The organic phase was washed with brine, filtered through a phase separator, and evaporated under vacuum. The crude material was purified by flash chromatography on Biotage silica NH cartridge (from 0% to 50% of EtOAc in c-Hex), affording the title compound (570 mg, 1.58 mmol, 90% yield).

LC-MS (ESI): m/z (M+1): 360.2 (Method 1)

### Intermediate 8: 6-(5-chloro-2-fluorophenyl)-3-(2,2-difluoroethoxy)-N-[(2,4-dimethoxyphenyl)methyl]pyridazin-4-amine

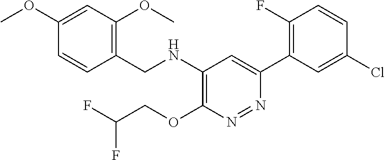 [SMILES: COc1ccc(CNc2cc(-c3cc(Cl)ccc3F)nnc2OCC(F)F)c(OC)c1]

In a round bottom flask, a mixture of Intermediate 7 (484 mg, 1.35 mmol), 5-chloro-2-fluorobenzeneboronic acid (352 mg, 2.02 mmol), Pd(dppf)Cl₂-DCM (197 mg, 0.27 mmol) and K₂CO₃ (558 mg, 4.04 mmol) in 1,4-Dioxane (8.2 mL) and H₂O (2.1 mL) was degassed (vacuum/N₂) and stirred at 110° C. for 2 hrs. The mixture was diluted with EtOAc, filtered through a Celite® pad, washing with EtOAc. The organic phase was washed with brine, separated, filtered through a phase separator, and evaporated under vacuum. The crude material was purified by flash chromatography on Biotage silica NH cartridge (from 0% to 20% of EtOAc in c-Hex), then further purified by flash chromatography on Biotage silica cartridge (from 0% to 2% of MeOH in DCM), to afford the title compound (417 mg, 0.92 mmol, 68% yield).

LC-MS (ESI): m/z (M+1): 454.2 (Method 1)

### Intermediate 9: 6-(5-chloro-2-fluorophenyl)-3-(2,2-difluoroethoxy)pyridazin-4-amine

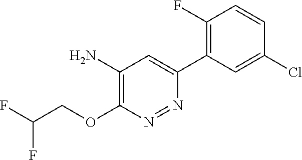 [SMILES: Nc1cc(-c2cc(Cl)ccc2F)nnc1OCC(F)F]

Intermediate 8 (332 mg, 0.73 mmol) was dissolved in a mixture DCM (6.4 mL)/TFA (1.6 mL) (8:2). The mixture was left stand at RT for 48 hrs. Volatiles were evaporated under vacuum. The residue material was charged on SCX (2 g) washing with MeOH, and eluting with 1 N NH₃ in MeOH. Basic fractions were evaporated to afford the title compound (223 mg, 0.73 mmol, recovery assumed quantitative).

LC-MS (ESI): m/z (M+1): 304.1 (Method 1)

### Intermediate 10: 6-chloro-N-[(2,4-dimethoxyphenyl)methyl]-3-[3-(methylsulfanyl)propoxy]pyridazin-4-amine

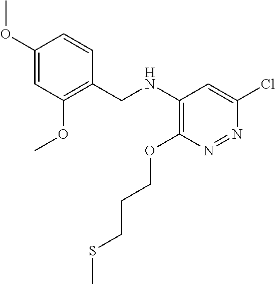 [SMILES: COc1ccc(CNc2cc(Cl)nnc2OCCCSC)c(OC)c1]

To a solution of 3-methylsulfanylpropan-1-ol (0.15 mL, 1.43 mmol) in DMF (2.1 mL), NaH 60% dispersion in oil (57 mg, 1.43 mmol) was added and the mixture was stirred at RT for 1.5 hrs (until gas evolution ceased). Then Intermediate 6 (150 mg, 0.48 mmol) dissolved in DMF (0.90 mL) was added and the mixture was stirred at 130° C. for 5 hrs. The mixture was allowed to reach the RT, poured into saturated NaHCO₃ aqueous solution and extracted with EtOAc. The organic phase was separated, filtered through a hydrophobic phase separator, and concentrated under vacuum. The crude material was purified by reverse flash chromatography on Biotage C18 cartridge (from H₂O+0.1% HCOOH to 70% MeCN+0.1% HCOOH). Evaporation of opportune fractions provided the title compound (50 mg, 0.13 mmol, 27% yield).

LC-MS (ESI): m/z (M+1): 384.2 (Method 1)

### Intermediate 11: 6-(5-chloro-2-fluorophenyl)-N-[(2,4-dimethoxyphenyl)methyl]-3-[3-(methylsulfanyl)propoxy]pyridazin-4-amine

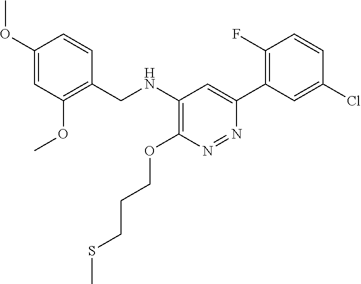 [SMILES: COc1ccc(CNc2cc(-c3cc(Cl)ccc3F)nnc2OCCCSC)c(OC)c1]

Intermediate 11 was prepared following the procedure used for the synthesis of Intermediate 8, starting from Intermediate 10 (45 mg, 0.12 mmol) and 5-chloro-2-fluorobenzeneboronic acid (27 mg, 0.15 mmol) in presence of Pd(dppf)Cl₂ (17 mg, 0.02 mmol) to afford title compound (40 mg, 0.08 mmol, 71% yield).

LC-MS (ESI): m/z (M+1): 478.2 (Method 1)

### Intermediate 12: 6-(5-chloro-2-fluorophenyl)-3-[3-(methylsulfanyl)propoxy]pyridazin-4-amine

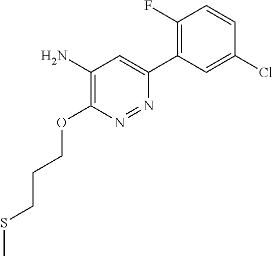 [SMILES: CSCCCOc1nnc(-c2cc(Cl)ccc2F)cc1N]

Intermediate 12 was prepared following the procedure used for the synthesis of Intermediate 9, starting from Intermediate 11 (40 mg, 0.08 mmol) to afford title compound (24 mg, 0.07 mmol, 87% yield). LC-MS (ESI): m/z (M+1): 328.1 (Method 1)

### Intermediate 13: 2-[(6-chloro-4-{[(2,4-dimethoxyphenyl)methyl]amino}pyridazin-3-yl)oxy]ethan-1-ol

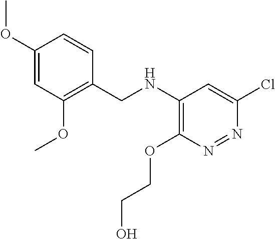 [SMILES: COc1ccc(CNc2cc(Cl)nnc2OCCO)c(OC)c1]

NaH, 60% dispersion in oil (140 mg, 3.5 mmol) was added portionwise to ethane-1,2-diol (8 mL, 3.18 mmol), stirred under N₂ at RT. After 30 minutes, Intermediate 6 (1 g, 3.18 mmol) was added. The reaction was heated at 100° C. for 1 h. After cooling down, the mixture was treated with cold water and extracted with EtOAc. Organic layer was separated, dried over Na₂SO₄, filtered and evaporated. The residue was purified by flash chromatography on Biotage silica NH cartridge (from DCM to 3% MeOH/0.3% H₂O) to afford the title compound (1.1 g, recovery assumed quantitative).

LC-MS (ESI): m/z (M+1): 340.1 (Method 1)

### Intermediate 14: 2-{2-[(6-chloro-4-{[(2,4-dimethoxyphenyl)methyl]amino}pyridazin-3-yl)oxy]ethyl}-2,3-dihydro-1H-isoindole-1,3-dione

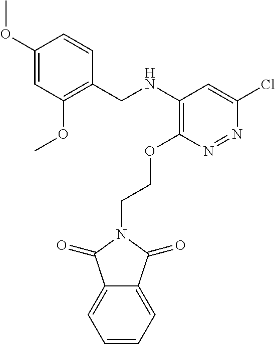 [SMILES: COc1ccc(CNc2cc(Cl)nnc2OCCN2C(=O)c3ccccc3C2=O)c(OC)c1]

Under N₂ atmosphere, diisopropyl azodicarboxylate (1.16 mL, 5.89 mmol) was added dropwise to a stirred solution of Intermediate 13 (1 g, 2.94 mmol), phthalimide (476 mg, 3.24 mmol) and PPh₃ (1.54 g, 5.89 mmol) in dry THE (20 mL), at RT. After 2 hrs, the solvent was removed by reduced pressure. The residue was treated with EtOH and the mixture was heated at reflux for 10 min. After cooling the solid was filtered and washed with EtOH/cyclohexane to afford the title compound (950 mg, 2.03 mmol, 69% yield). LC-MS (ESI): m/z (M+1): 469.2 (Method 1)

### Intermediate 15: 2-[(2-{[6-(5-chloro-2-fluorophenyl)-4-{1[(2,4-dimethoxyphenyl)methyl]amino}pyridazin-3-yl]oxy}ethyl)carbamoyl]benzoic acid

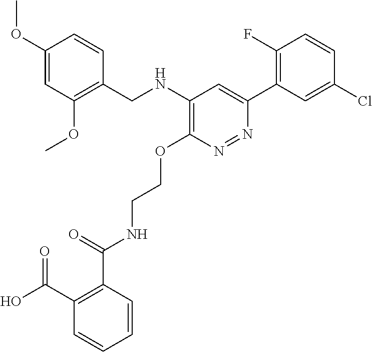 [SMILES: COc1ccc(CNc2cc(-c3cc(Cl)ccc3F)nnc2OCCNC(=O)c2ccccc2C(=O)O)c(OC)c1]

K₂CO₃ (989 mg, 7.16 mmol) was added to a stirred mixture of Intermediate 14 (1.13 g, 2.39 mmol), 5-chloro-2-fluorobenzeneboronic acid (624 mg, 3.58 mmol) and Pd(dppf)Cl₂ (350 mg, 0.48 mmol) in 1,4-dioxane (74.6 mL) and H₂O (18.6 mL). The reaction was degassed by N₂ bubbling, then the vial was closed and heated at 110° C. for 2 hrs. After cooling down, the mixture was diluted with EtOAc and H₂O. Phases were separated and the aqueous one was treated with citric acid aqueous solution and extracted with EtOAc. Organic layer was separated, dried over Na₂SO₄, filtered, and evaporated to afford the title compound (330 mg, 0.57 mmol, 24% yield).

LC-MS (ESI): m/z (M+1): 581.3 (Method 1)

### Intermediate 16: 2-(2-{[6-(5-chloro-2-fluorophenyl)-4-{[(2,4-dimethoxyphenyl)methyl]amino}pyridazin-3-yl]oxy}ethyl)-2,3-dihydro-1H-isoindole-1,3-dione

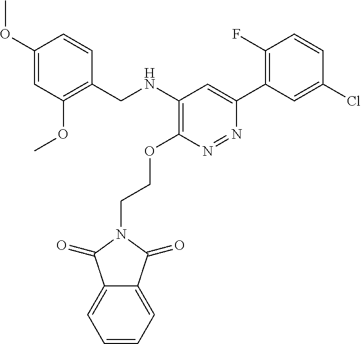 [SMILES: COc1ccc(CNc2cc(-c3cc(Cl)ccc3F)nnc2OCCN2C(=O)c3ccccc3C2=O)c(OC)c1]

DIPEA (556 μL, 3.2 mmol) was added to a stirred mixture of Intermediate 14 (750 mg, 1.6 mmol), 5-chloro-2-fluorobenzeneboronic acid (558 mg, 3.2 mmol) and Pd(PPh₃)₄ (92 mg, 0.08 mmol) in 1,4-dioxane (30 mL) at RT. The reaction was degassed by N₂ bubbling. The vial was closed and heated at 110° C. for 20 hrs. After cooling, the solvent was removed by reduced pressure. The residue was treated with EtOAc and washed with H₂O. Organic layer was separated, dried over Na₂SO₄, filtered and evaporated. The residue was purified by flash chromatography on Biotage silica NH cartridge (from 0% to 50% of EtOAc in c-Hex) to afford the title compound (760 mg, 1.35 mmol, 84% yield).

LC-MS (ESI): m/z (M+1): 469.2 (Method 1)

### Intermediate 17: 2-(2-{[4-amino-6-(5-chloro-2-fluorophenyl)pyridazin-3-yl]oxy}ethyl)-2,3-dihydro-1H-isoindole-1,3-dione

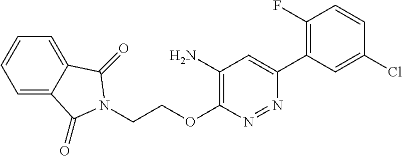 [SMILES: Nc1cc(-c2cc(Cl)ccc2F)nnc1OCCN1C(=O)c2ccccc2C1=O]

### Method A

Intermediate 15 (330 mg, 0.40 mmol) was treated with 4N HCl in 1,4-dioxane (3.0 mL) at 90° C. for 7 hrs. The solvent was removed by reduced pressure. The residue was treated with aqueous NaHCO₃ and extracted with EtOAc. Organic layer was separated, dried over Na₂SO₄, filtered and evaporated to afford the title compound (100 mg, 0.22 mmol, 61% yield).

### Method B

TFA (3 mL, 39.2 mmol) was added to a stirred solution of Intermediate 16 (720 mg, 1.23 mmol) in DCM (4 mL) at RT under N₂. The reaction was stirred for 40 hrs. The solvents were removed by reduced pressure. The residue was treated with water and washed with EtOAc and organic layer was discarded. Aqueous phase was treated with 33% NH₄OH in H₂O until pH 10 and extracted with DCM. Organic layer was separated, dried over Na₂SO₄ and evaporated to afford the title compound (360 mg, 0.87 mmol, 71% yield). LC-MS (ESI): m/z (M+1): 413.1 (Method 1)

### Intermediate 18: 2-(2-{[6-(5-chloro-2-fluorophenyl)-4-({7-[2-(4-methylpiperazin-1-yl)ethoxy]quinolin-4-yl}amino)pyridazin-3-yl]oxy}ethyl)-2,3-dihydro-1H-isoindole-1,3-dione

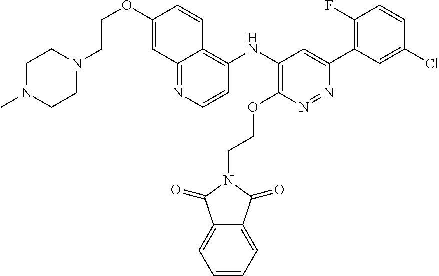 [SMILES: CN1CCN(CCOc2ccc3c(Nc4cc(-c5cc(Cl)ccc5F)nnc4OCCN4C(=O)c5ccccc5C4=O)ccnc3c2)CC1]

Cs₂CO₃ (238 mg, 0.73 mmol) was added to a stirred mixture of Intermediate 17 (150 mg, 0.36 mmol), Intermediate 5 (122 mg, 0.40 mmol), Pd(OAc)₂ (4 mg, 0.02 mmol) and Xantphos (21 mg, 0.40 mmol) in 1,4-dioxane (10 mL) at RT. The mixture was degassed by N₂ bubbling, the vial was closed and irradiated at 110° C. in MW apparatus for 2 hrs. After cooling, the mixture was filtered on Celite® pad washing with EtOAc. The solvents were removed by reduced pressure and the residue was purified by flash chromatography on Biotage silica NH (cartridge (from DCM to 2% MeOH/0.2% H₂O) to afford the title compound (170 mg, 0.25 mmol, 69% yield).

LC-MS (ESI): m/z (M+1): 682.4 (Method 2)

### Intermediate 19: 6-chloro-N-[(2,4-dimethoxyphenyl)methyl]-3-(3-methanesulfonylpropoxy)pyridazin-4-amine

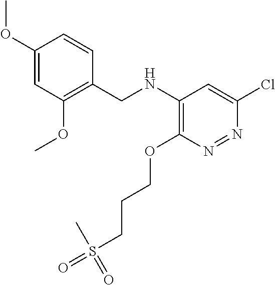 [SMILES: COc1ccc(CNc2cc(Cl)nnc2OCCCS(C)(=O)=O)c(OC)c1]

Intermediate 19 was prepared following the procedure used for the synthesis of Intermediate 10, starting from Intermediate 6 (300 mg, 0.95 mmol) and 3-(methylsulfonyl)-1-propanol (396 mg, 2.86 mmol) at 110° C. to afford title compound (65 mg, 0.16 mmol, 16% yield). LC-MS (ESI): m/z (M+1): 416.1 (Method 1)

### Intermediate 20: 6-(5-chloro-2-fluorophenyl)-N-[(2,4-dimethoxyphenyl)methyl]-3-(3-methanesulfonylpropoxy)pyridazin-4-amine

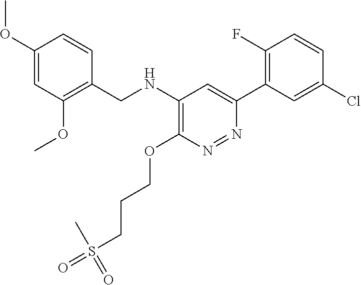 [SMILES: COc1ccc(CNc2cc(-c3cc(Cl)ccc3F)nnc2OCCCS(C)(=O)=O)c(OC)c1]

Intermediate 20 was prepared following the procedure used for the synthesis of Intermediate 8, starting from Intermediate 19 (65 mg, 0.16 mmol) and 5-chloro-2-fluorobenzeneboronic acid (35 mg, 0.20 mmol) in presence of Pd(dppf)Cl₂ (23 mg, 0.03 mmol) to afford title compound (45 mg, 0.09 mmol, 56% yield).

LC-MS (ESI): m/z (M+1): 510.1 (Method 1)

### Intermediate 21: 6-(5-chloro-2-fluorophenyl)-3-(3-methanesulfonylpropoxy)pyridazin-4-amine

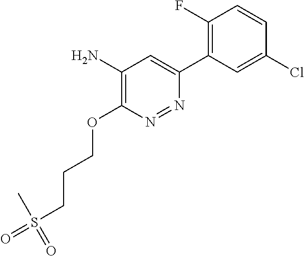 [SMILES: CS(=O)(=O)CCCOc1nnc(-c2cc(Cl)ccc2F)cc1N]

Intermediate 21 was prepared following the procedure used for the synthesis of Intermediate 9, starting from 6-(5-chloro-2-fluorophenyl)-N-[(2,4-dimethoxyphenyl)methyl]-3-(3-methanesulfonylpropoxy)pyridazin-4-amine (Intermediate 20, 45 mg, 0.09 mmol) to afford title compound (30 mg, 0.08 mmol, 95% yield). LC-MS (ESI): m/z (M+1): 360.0 (Method 2)

### Intermediate 22: N-(4-{[6-(5-chloro-2-fluorophenyl)-3-[2-(1,3-dioxo-2,3-dihydro-1H-isoindol-2-yl)ethoxy]pyridazin-4-yl]amino}pyridin-2-yl)-3-(4-methylpiperazin-1-yl)propanamide

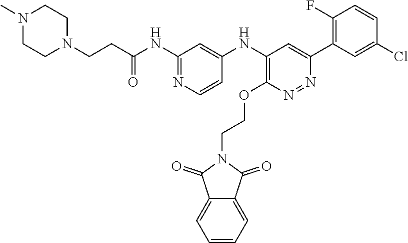 [SMILES: CN1CCN(CCC(=O)Nc2cc(Nc3cc(-c4cc(Cl)ccc4F)nnc3OCCN3C(=O)c4ccccc4C3=O)ccn2)CC1]

Intermediate 22 was prepared following the procedure used for the synthesis of Intermediate 18, starting from 2-(2-{[4-amino-6-(5-chloro-2-fluorophenyl)pyridazin-3-yl]oxy}ethyl)-2,3-dihydro-1H-isoindole-1,3-dione (Intermediate 17, 200 mg, 0.48 mmol) and N-(4-bromopyridin-2-yl)-3-(4-methylpiperazin-1-yl)propanamide (Intermediate 2, 190 mg, 0.58 mmol) to afford title compound (120 mg, 0.18 mmol, 38% yield).

LC-MS (ESI): m/z (M+1): 659.4 (Method 2)

### Intermediate 23: methyl 4-chloro-1-{[2-(trimethylsilyl)ethoxy]methyl}-1H-pyrrolo[2,3-b]pyridine-2-carboxylate

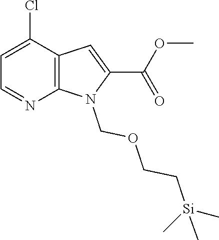 [SMILES: COC(=O)c1cc2c(Cl)ccnc2n1COCC[Si](C)(C)C]

To an ice-cooled suspension of methyl 4-chloro-1H-pyrrolo[2,3-b]pyridine-2-carboxylate (1.0 g, 4.75 mmol) in dry THE (35 mL), NaH 60% dispersion in oil (0.28 g, 7.12 mmol) was added and the mixture stirred for 30 min before adding 2-(chloromethoxy)ethyl-trimethylsilane (1.09 mL, 6.17 mmol). The reaction mixture was allowed to reach RT and stirred at RT for 3 hrs. The mixture was quenched with saturated NH₄Cl aqueous solution, diluted with EtOAc and washed with brine (1×). The organic phase was dried and concentrated under vacuum and left as solid at RT, overnight. The day after, UPLC check showed the complete conversion to give the reported regioisomer. The residue was purified by flash chromatography on Biotage silica cartridge (from c-Hex to 10% EtOAc), to afford the title compound (820 mg, 2.41 mmol, 51% yield).

LC-MS (ESI): m/z (M+1): 341.1 (Method 1)

¹H NMR (500 MHz, Chloroform-d) δ ppm 8.38 (d, J=5.1 Hz, 1H), 7.40 (s, 1H), 7.20 (d, J=5.1 Hz, 1H), 6.14 (s, 2H), 3.97 (s, 3H), 3.52-3.58 (m, 2H), 0.85-0.92 (m, 2H), −0.11-−0.05 (m, 9H).

### Intermediate 24: 6-chloro-N-[(2,4-dimethoxyphenyl)methyl]-3-[3-(dimethylamino)propoxy]pyridazin-4-amine

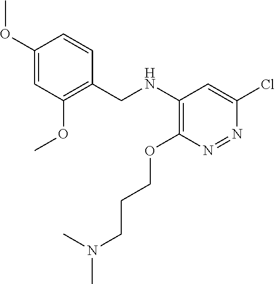

Intermediate 24 was prepared following the procedure used for the synthesis of Intermediate 7, starting from 3,6-dichloro-N-[(2,4-dimethoxyphenyl)methyl]pyridazin-4-amine (Intermediate 6, 1 g, 3.18 mmol) and 3-(dimethylamino)propan-1-ol (0.49 mL, 4.14 mmol) at 120° C., to afford title compound (400 mg, 1.05 mmol, 33% yield).

LC-MS (ESI): m/z (M+1): 381.2 (Method 1)

### Intermediate 25: 6-(5-chloro-2-fluorophenyl)-N-[(2,4-dimethoxyphenyl)methyl]-3-[3-(dimethylamino)propoxy]pyridazin-4-amine

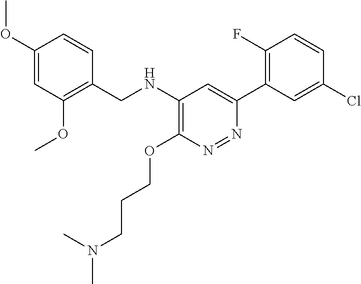

Intermediate 25 was prepared following the procedure used for the synthesis of Intermediate 8, starting from 6-chloro-N-[(2,4-dimethoxyphenyl)methyl]-3-[3-(dimethylamino)propoxy]pyridazin-4-amine (Intermediate 24, 400 mg, 1.05 mmol) and 5-chloro-2-fluorobenzeneboronic acid (275 mg, 1.58 mmol) in presence of Pd(dppf)Cl₂ (154 mg, 0.21 mmol) to afford title compound (250 mg, 0.53 mmol, 50% yield).

LC-MS (ESI): m/z (M+1): 475.0 (Method 1)

### Intermediate 26: 6-(5-chloro-2-fluorophenyl)-3-[3-(dimethylamino)propoxy]pyridazin-4-amine

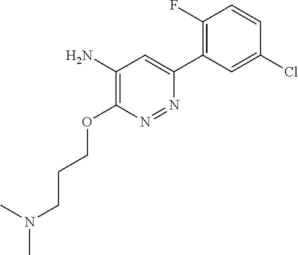 [SMILES: CN(C)CCCOc1nnc(-c2cc(Cl)ccc2F)cc1N]

Intermediate 26 was prepared following the procedure used for the synthesis of Intermediate 9, starting from 6-(5-chloro-2-fluorophenyl)-N-[(2,4-dimethoxyphenyl)methyl]-3-[3-(dimethylamino)propoxy]pyridazin-4-amine (Intermediate 25, 250 mg, 0.53 mmol) to afford title compound (150 mg, 0.46 mmol, 88% yield). LC-MS (ESI): m/z (M+1): 325.3 (Method 2)

### Intermediate 27: methyl 4-{[6-(5-chloro-2-fluorophenyl)-3-[3-(dimethylamino)propoxy]pyridazin-4-yl]amino}-1-{[2-(trimethylsilyl)ethoxy]methyl}-1H-pyrrolo[2,3-b]pyridine-2-carboxylate

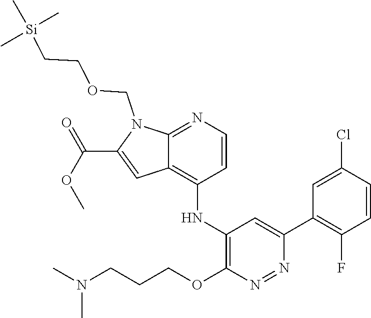

Intermediate 27 was prepared following the procedure used for the synthesis of Intermediate 18, starting from 6-(5-chloro-2-fluorophenyl)-3-[3-(dimethylamino)propoxy]pyridazin-4-amine (Intermediate 26, 80 mg, 0.24 mmol) and methyl 4-chloro-1-{[2-(trimethylsilyl)ethoxy]methyl}-1H-pyrrolo[2,3-b]pyridine-2-carboxylate (Intermediate 23, 93 mg, 0.26 mmol) to afford title compound (70 mg, 0.11 mmol, 47% yield). LC-MS (ESI): m/z (M+1): 629.5 (Method 1)

### Intermediate 28: 6-chloro-N-[(2,4-dimethoxyphenyl)methyl]-3-[2-(dimethylamino)ethoxy]pyridazin-4-amine

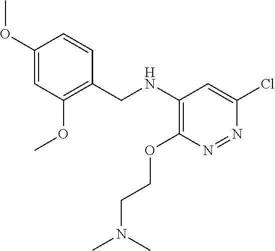

Intermediate 28 was prepared following the procedure used for the synthesis of Intermediate 10, starting from 3,6-dichloro-N-[(2,4-dimethoxyphenyl)methyl]pyridazin-4-amine (Intermediate 6, 700 mg, 2.23 mmol) and 2-(dimethylamino)ethanol (0.67 mL, 6.68 mmol) to afford title compound (850 mg, recovery assumed quantitative).

LC-MS (ESI): m/z (M+1): 367.2 (Method 1)

### Intermediate 29: 6-(5-chloro-2-fluorophenyl)-N-[(2,4-dimethoxyphenyl)methyl]-3-[2-(dimethylamino)ethoxy]pyridazin-4-amine

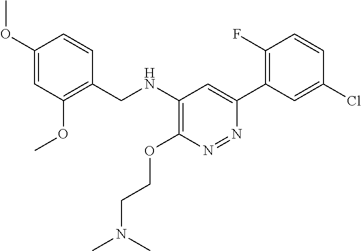

Intermediate 29 was prepared following the procedure used for the synthesis of Intermediate 8, starting from 6-chloro-N-[(2,4-dimethoxyphenyl)methyl]-3-[2-(dimethylamino)ethoxy]pyridazin-4-amine (Intermediate 28, 850 mg, 2.23 mmol) and 5-chloro-2-fluorobenzeneboronic acid (606 mg, 3.48 mmol) in presence of Pd(dppf)Cl₂ (339 mg, 0.46 mmol) to afford title compound (600 mg, 1.30 mmol, 56% yield).

LC-MS (ESI): m/z (M+1): 461.8 (Method 2)

### Intermediate 30: 6-(5-chloro-2-fluorophenyl)-3-[2-(dimethylamino)ethoxy]pyridazin-4-amine

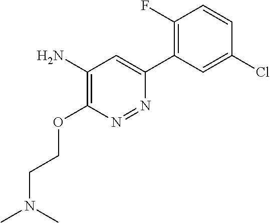

Intermediate 30 was prepared following the procedure used for the synthesis of Intermediate 9, starting from Intermediate 29 (600 mg, 1.30 mmol) to afford title compound (330 mg, 1.06 mmol, 82% yield). LC-MS (ESI): m/z (M+1): 311.5 (Method 2).

### Intermediate 31: 1-tert-butyl 3-methyl 4-methylpiperazine-1,3-dicarboxylate

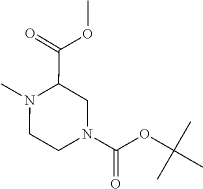

To a suspension methyl-4-boc-piperazine-2-carboxylate (150 mg, 0.61 mmol) in MeOH (2.05 mL), acetic acid (0.11 mL, 1.84 mmol) and formaldehyde 37% w/w in water (0.23 mL, 3.07 mmol) were added. This mixture was stirred at RT for 30 min, before adding sodium cyanoborohydride (77 mg, 1.23 mmol). The suspension quickly turned into a solution. After 1 h volatiles were removed under vacuum. The residue was taken up with DCM, washed with saturated NaHCO₃ aqueous solution. The organic phase was filtered through a phase separator and concentrated under vacuum. The crude material was purified by flash chromatography on Biotage silica cartridge (from 0% to 5% of MeOH in DCM) to afford the title compound (124 mg, 0.48 mmol, 78% yield).

LC-MS (ESI): m/z (M+1): 258.5 (Method 1)

### Intermediate 32: methyl 1-methylpiperazine-2-carboxylate dihydrochloride

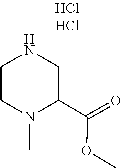

A solution of 1-tert-butyl 3-methyl 4-methylpiperazine-1,3-dicarboxylate (Intermediate 31 (1.15 g, 4.45 mmol) in HCl solution, 4 M in 1,4-dioxane (5.6 mL, 22.3 mmol) and MeOH (11 mL) was stirred at RT for 2 hrs. Volatiles were removed under vacuum, to afford the title compound (Intermediate 32, 1.3 g, recovery assumed quantitative) that was used in the next step without further purification.

LC-MS (ESI): m/z (M free base+1): 159.1 (Method 2)

### Intermediate 33: N-(4-bromopyridin-2-yl)-2-chloroacetamide

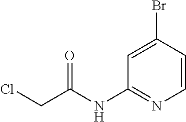 [SMILES: O=C(CCl)Nc1cc(Br)ccn1]

2-chloroacetyl chloride (0.25 mL, 3.18 mmol) was added dropwise to a solution of 4-bromo-2-pyridinamine (500 mg, 2.89 mmol) and TEA (1.21 mL, 8.67 mmol) in dry DCM (14.5 mL) at 0° C. The mixture was stirred at RT for 3 hrs. The mixture was diluted with DCM, washed with saturated NaHCO₃ aqueous solution and with brine. The organic phase was filtered through a phase separator and evaporated under vacuum. The crude material was purified by flash chromatography on Biotage silica cartridge (from DCM to 10% EtOAc) to afford the title compound (470 mg, 1.88 mmol, 65% yield).

LC-MS (ESI): m/z (M+1): 249.0 (Method 2)

### Intermediate 34: methyl 4-{[(4-bromopyridin-2-yl)carbamoyl]methyl}-1-methylpiperazine-2-carboxylate

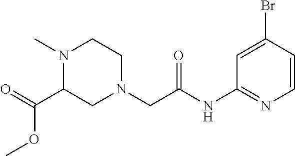

To a stirred suspension of N-(4-bromopyridin-2-yl)-2-chloroacetamide (Intermediate 33, 250 mg, 1 mmol) and K₂CO₃ (692 mg, 5.01 mmol) in DMF (5 mL), methyl 1-methylpiperazine-2-carboxylate dihydrochloride (Intermediate 32, 347 mg, 1.5 mmol) was added. The reaction was stirred at RT overnight. The mixture was diluted with EtOAc and washed with s. NaHCO₃ saturated solution (3×) and brine (1×). The organic phase was filtered through a phase separator and concentrated under vacuum. The crude material was purified by flash chromatography on Biotage silica NH cartridge (from 0% to 25% of EtOAc in c-Hex) to afford the title compound (240 mg, 0.65 mmol, 64% yield).

LC-MS (ESI): m/z (M+1): 371.4 (Method 2)

### Intermediate 35: 6-chloro-N-[(2,4-dimethoxyphenyl)methyl]-3-methoxypyridazin-4-amine

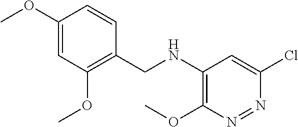 [SMILES: COc1ccc(CNc2cc(Cl)nnc2OC)c(OC)c1]

Sodium methoxide 25% in MeOH (0.8 mL, 3.5 mmol) was added to a suspension of 3,6-dichloro-N-[(2,4-dimethoxyphenyl)methyl]pyridazin-4-amine (Intermediate 6, 1 g, 3.18 mmol) in MeOH (10.64 mL). The vial was sealed and irradiated at 120° C. for 1 h with MW apparatus. Volatiles were removed under vacuum to afford the title compound (1.3 g, NaCl inside, recovery assumed quantitative) used as such for the next step.

LC-MS (ESI): m/z (M+1): 310.5 (Method 2)

### Intermediate 36: 6-(5-chloro-2-fluorophenyl)-N-[(2,4-dimethoxyphenyl)methyl]-3-methoxypyridazin-4-amine

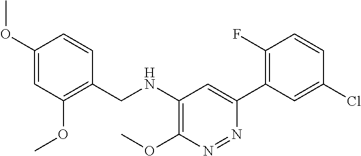 [SMILES: COc1ccc(CNc2cc(-c3cc(Cl)ccc3F)nnc2OC)c(OC)c1]

Intermediate 36 was prepared following the procedure used for the synthesis of Intermediate 8, starting from 6-chloro-N-[(2,4-dimethoxyphenyl)methyl]-3-methoxypyridazin-4-amine (Intermediate 35, 3.18 mmol) and 5-chloro-2-fluorobenzeneboronic acid (832 mg, 4.77 mmol) in presence of Pd(dppf)Cl₂ (339 mg, 0.46 mmol) to afford title compound (630 mg, 1.56 mmol, 49% yield).

LC-MS (ESI): m/z (M+1): 404.2 (Method 1)

### Intermediate 37: 6-(5-chloro-2-fluorophenyl)-3-methoxypyridazin-4-amine

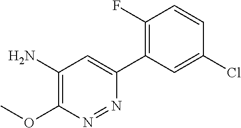 [SMILES: COc1nnc(-c2cc(Cl)ccc2F)cc1N]

Intermediate 37 was prepared following the procedure used for the synthesis of Intermediate 9, starting from 6-(5-chloro-2-fluorophenyl)-N-[(2,4-dimethoxyphenyl)methyl]-3-methoxypyridazin-4-amine (Intermediate 36, 630 mg, 1.56 mmol) to afford title compound (387 mg, 1.53 mmol, 98% yield).

LC-MS (ESI): m/z (M+1): 254.1 (Method 1)

### Intermediate 38: N-(4-bromopyridin-2-yl)-3-[4-(2,2,2-trifluoroethyl)piperazin-1-yl]propanamide

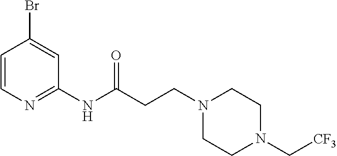 [SMILES: O=C(CCN1CCN(CC(F)(F)F)CC1)Nc1cc(Br)ccn1]

Intermediate 38 was prepared following the procedure used for the synthesis of Intermediate 2, starting from N-(4-bromopyridin-2-yl)prop-2-enamide (Intermediate 1, 200 mg, 0.88 mmol) and 1-(2,2,2-trifluoroethyl)piperazine (200 mg, 1.19 mmol) to afford title compound (346 mg, 0.86 mmol, 99%). LC-MS (ESI): m/z (M+1): 395.2 (Method 1)

### Intermediate 39: tert-butyl 4-{[(4-bromopyridin-2-yl)carbamoyl]methyl}piperazine-1-carboxylate

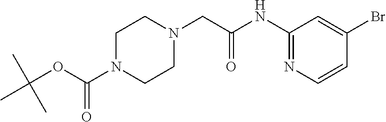 [SMILES: CC(C)(C)OC(=O)N1CCN(CC(=O)Nc2cc(Br)ccn2)CC1]

A mixture of N-(4-bromopyridin-2-yl)-2-chloroacetamide (Intermediate 33, 759 mg, 3.08 mmol), 1-piperazinecarboxylic acid tert-butyl ester (1.15 g, 6.16 mmol) and K₂CO₃ (1.28 g, 9.25 mmol) in dry DMF (15 mL) was stirred under N₂ at RT overnight. The mixture was poured into saturated NaHCO₃ aqueous solution and extracted with EtOAc. The organic phase was separated, filtered through a hydrophobic phase separator, and concentrated at reduced pressure. The solvent was evaporated, the crude material was purified by flash chromatography on Biotage silica NH cartridge (from c-Hex to 45% EtOAc) to afford the title compound (1.05 g, 2.64 mmol, 86% yield).

LC-MS (ESI): m/z (M+1): 399.2 (Method 1)

### Intermediate 40: N-(4-bromopyridin-2-yl)-2-(piperazin-1-yl)acetamide

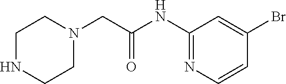 [SMILES: O=C(CN1CCNCC1)Nc1cc(Br)ccn1]

tert-butyl 4-{[(4-bromopyridin-2-yl)carbamoyl]methyl}piperazine-1-carboxylate (Intermediate 39, 1.05 g, 2.64 mmol) was dissolved in a mixture of DCM (20 mL) and TFA (5.05 mL, 66.1 mmol) and stirred at RT for 1 h. Volatiles were evaporated at reduced pressure and the residue was charged on a SCX cartridge (10 g), washed with MeOH and eluted with 2N NH₃ solution in MeOH. Basic fractions were evaporated at reduced pressure to provide the title compound (795 mg, 2.66 mmol, recovery assumed quantitative). The material was used in the next step without further purification.

LC-MS (ESI): m/z (M+1): 299.1 (Method 2)

### Intermediate 41: N-(4-bromopyridin-2-yl)-2-[4-(2,2,2-trifluoroethyl)piperazin-1-yl]acetamide

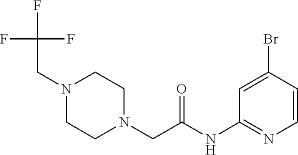 [SMILES: O=C(CN1CCN(CC(F)(F)F)CC1)Nc1cc(Br)ccn1]

To a mixture of N-(4-bromopyridin-2-yl)-2-(piperazin-1-yl)acetamide (Intermediate 40, 200 mg, 0.67 mmol) and TEA (0.14 mL, 1 mmol) in THE (6 mL), trifluoromethanesulfonic acid 2,2,2-trifluoroethyl ester (0.11 mL, 0.74 mmol) was added and the mixture was stirred at RT overnight. Volatiles were removed at reduced pressure and the crude was purified by flash chromatography on Biotage silica NH cartridge (from c-Hex to 30% EtOAc). Evaporation of proper fractions provided the title compound (204 mg, 0.54 mmol, 80% yield). LC-MS (ESI): m/z (M+1): 381.3 (Method 2)

### Intermediate 42: (3-methyloxetan-3-yl)methyl 2-(acetyloxy)acetate

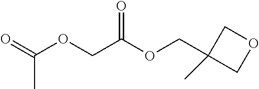

### Step 1

To an ice-cooled solution of 2-hydroxyacetic acid (1.5 g, 19.7 mmol) in Pyridine (6 mL), acetic acid acetyl ester (1.92 mL, 20.3 mmol) was added then the mixture was allowed to reach RT and stirred overnight. The mixture was partitioned between EtOAc and saturated NaHCO₃ aqueous solution, then separated. The aqueous layer was adjusted to pH=2-3 with 1N HCl and extracted with EtOAc (2×90 mL) and DCM (2×90 mL). Combined organic phases were dried over Na₂SO₄ and concentrated at reduced pressure to provide a crude containing 2-(acetyloxy)acetic acid (1.41 g, 11.9 mmol, 60% yield). The material was used in the next step without further purification.

¹H NMR (400 MHz, DMSO-d₆) δ ppm 4.53 (s, 2H), 2.07 (s, 3H).

### Step 2

To a solution of 2-acetyloxyacetic acid (1.41 g, 11.9 mmol), (3-methyl-3-oxetanyl)methanol (1.78 mL, 17.9 mmol) and DMAP (145 mg, 1.19 mmol) in DCM (22 mL), N-Ethyl-N′-(3-dimethylaminopropyl)carbodiimide hydrochloride (3.65 g, 19.0 mmol) was added and the mixture was stirred at RT overnight. The mixture was washed with saturated NaHCO₃ aqueous solution, 0.1N HCl, and finally with brine. The organic phase was separated, filtered through a hydrophobic phase separator and concentrated at reduced pressure. The crude was purified by flash chromatography on Biotage silica cartridge (from DCM to 4% MeOH). Evaporation of proper fractions provided the title compound (1.90 g, 9.38 mmol, 79% yield) as a colourless oil that slowly solidifies.

LC-MS (ESI): m/z (M+1): 203.0 (Method 1)

¹H NMR (400 MHz, Chloroform-d) δ ppm 4.67 (s, 2H), 4.53 (d, J=5.79 Hz, 2H), 4.41 (d, J=6.06 Hz, 2H), 4.28 (s, 2H), 2.19 (s, 3H), 1.36 (s, 3H).

### Intermediate 43: {4-methyl-2,6,7-trioxabicyclo[2.2.2]octan-1-yl}methyl acetate

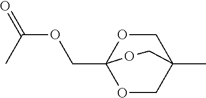

To an ice-cooled solution of ((3-methyloxetan-3-yl)methyl 2-(acetyloxy)acetate (Intermediate 42, 1.9 g, 9.38 mmol) in DCM (20 mL), boron trifluoride diethyl etherate (0.12 mL, 0.94 mmol) was slowly added. The mixture was allowed to reach RT and stirred for 4 hrs. The reaction was cooled to 0° C. and quenched with TEA (1.5 eq) stirring for 15 minutes. The mixture was diluted with DCM and washed with water (2×) and brine (2×) then the organic phase was separated, filtered through a hydrophobic phase separator and concentrated at reduced pressure. The crude was purified by flash chromatography on Biotage silica cartridge (from c-Hex to 70% EtOAc). Evaporation of proper fractions provided the title compound (1.14 g, 5.63 mmol, 60% yield).

LC-MS (ESI): m/z (M+1): 203.0 (Method 1)

¹H NMR (400 MHz, Chloroform-d) δ ppm 4.13 (s, 2H), 3.97 (s, 6H), 2.15 (s, 3H), 0.85 (s, 3H).

### Intermediate 44: {4-methyl-2,6,7-trioxabicyclo[2.2.2]octan-1-yl}methanol

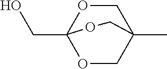

To an ice-cooled solution of {4-methyl-2,6,7-trioxabicyclo[2.2.2]octan-1-yl}methyl acetate (Intermediate 43, 1.14 g, 5.63 mmol) in MeOH (20 mL), NaH 60% dispersion in oil (22.5 mg, 0.56 mmol) was added and the mixture was allowed to reach RT and stirred for 2 hrs. Volatiles were removed at reduced pressure and the crude was purified by flash chromatography on Biotage silica cartridge (from DCM to 4% MeOH). Evaporation of proper fractions provided the title compound (768 mg, 4.79 mmol, 85% yield). LC-MS (ESI): m/z (M+1): 161.0 (Method 1)

¹H NMR (400 MHz, Chloroform-d) δ ppm 3.98 (s, 6H), 3.60 (d, J=6.77 Hz, 2H), 1.87 (t, J=6.81 Hz, 1H), 0.86 (s, 3H).

### Intermediate 45: 6-chloro-3-({4-methyl-2,6,7-trioxabicyclo[2.2.2]octan-1-yl}methoxy)pyridazin-4-amine

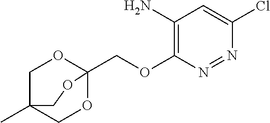 [SMILES: CC12COC(COc3nnc(Cl)cc3N)(OC1)OC2]

Intermediate 45 was prepared following the procedure used for the synthesis of Intermediate 10, starting from 3,6-dichloropyridazin-4-amine (116 mg, 0.71 mmol) and 2{4-methyl-2,6,7-trioxabicyclo[2.2.2]octan-1-yl}methanol (Intermediate 44, 340 mg, 2.12 mmol) to afford title compound (320 mg, recovery assumed quantitative).

LC-MS (ESI): m/z (M+1): 288.0 (Method 2)

### Intermediate 46: 6-(5-chloro-2-fluorophenyl)-3-({4-methyl-2,6,7-trioxabicyclo[2.2.2]octan-1-yl}methoxy)pyridazin-4-amine

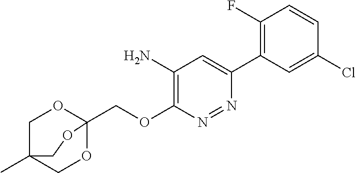 [SMILES: CC12COC(COc3nnc(-c4cc(Cl)ccc4F)cc3N)(OC1)OC2]

Intermediate 46 was prepared following the procedure used for the synthesis of Intermediate 8, starting from 6-chloro-3-({4-methyl-2,6,7-trioxabicyclo[2.2.2]octan-1-yl}methoxy)pyridazin-4-amine (Intermediate 45, 0.71 mmol) and 5-chloro-2-fluorobenzeneboronic acid (185 mg, 1.06 mmol) in presence of Pd(dppf)Cl₂ (103 mg, 0.14 mmol) to afford title compound (117 mg, 0.31 mmol, 43% yield).

LC-MS (ESI): m/z (M+1): 382.1 (Method 1)

### Intermediate 47: N-(4-{[6-(5-chloro-2-fluorophenyl)-3-({4-methyl-2,6,7-trioxabicyclo[2.2.2]octan-1-yl}methoxy)pyridazin-4-yl]amino}pyridin-2-yl)-3-(4-methylpiperazin-1-yl)propanamide

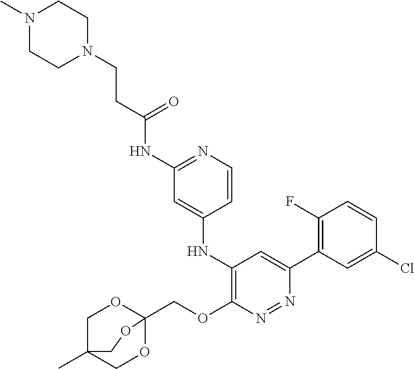 [SMILES: CN1CCN(CCC(=O)Nc2cc(Nc3cc(-c4cc(Cl)ccc4F)nnc3OCC34OCC(C)(CO3)CO4)ccn2)CC1]

A mixture of 6-(5-chloro-2-fluorophenyl)-3-({4-methyl-2,6,7-trioxabicyclo[2.2.2]octan-1-yl}methoxy)pyridazin-4-amine (Intermediate 46, 50 mg, 0.13 mmol), N-(4-bromopyridin-2-yl)-3-(4-methylpiperazin-1-yl)propanamide (Intermediate 2, 48 mg, 0.14 mmol), Pd(OAc)₂ (1.8 mg, 0.01 mmol), Xantphos (9.1 mg, 0.02 mmol) and Cs₂CO₃ (85 mg, 0.26 mmol) in 1,4-dioxane (1 mL) was degassed (vacuum/N₂) and heated at 100° C. for 2 hrs. The mixture was filtered through a Celite® pad washing with EtOAc; the filtrate concentrated at reduced pressure and the crude was purified by flash chromatography on Biotage silica NH cartridge (from DCM to 2% MeOH). Proper fraction were collected and purified by preparative HPLC to afford the title compound (41 mg, 0.07 mmol, 51% yield). LC-MS (ESI): m/z (M+1): 628.2 (Method 2)

### Intermediate 48: 6-chloro-N-[(2,4-dimethoxyphenyl)methyl]-3-(methylsulfanyl)pyridazin-4-amine

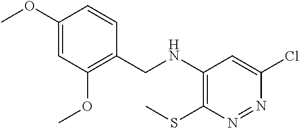 [SMILES: COc1ccc(CNc2cc(Cl)nnc2SC)c(OC)c1]

A mixture of 3,6-dichloro-N-[(2,4-dimethoxyphenyl)methyl]pyridazin-4-amine (Intermediate 6, 450 mg, 1.43 mmol) and sodium thiomethoxide (250 mg, 3.58 mmol) in DMF (9.6 mL) was stirred at RT for 16 hrs. The mixture was diluted with saturated NaHCO₃ aqueous solution and extracted with DCM. The organic phase was dried with Na₂SO₄, filtered and concentrated under reduced pressure. The crude product was purified by flash chromatography on Biotage silica cartridge (from c-Hex to 50% EtOAc) and then further purified by reverse flash chromatography on Biotage C18 cartridge (from H₂O +0.1% HCOOH to 97% MeCN+0.1% HCOOH). Proper fractions were collected and diluted with DCM, washed with saturated NaHCO₃ aqueous solution and concentrated under reduced pressure to afford the title compound (240 mg, 0.74 mmol, 51% yield).

LC-MS (ESI): m/z (M+1): 326.1 (Method 1)

### Intermediate 49: 6-(5-chloro-2-fluorophenyl)-N-[(2,4-dimethoxyphenyl)methyl]-3-(methylsulfanyl)pyridazin-4-amine

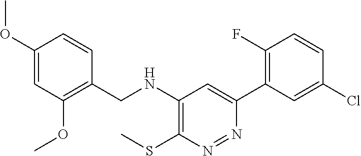 [SMILES: COc1ccc(CNc2cc(-c3cc(Cl)ccc3F)nnc2SC)c(OC)c1]

Intermediate 49 was prepared following the procedure used for the synthesis of Intermediate 8, starting from 6-chloro-N-[(2,4-dimethoxyphenyl)methyl]-3-(methylsulfanyl)pyridazin-4-amine (Intermediate 48, 240 mg, 0.74 mmol) and 5-chloro-2-fluorobenzeneboronic acid (167 mg, 0.96 mmol) in presence of Pd(dppf)Cl₂ (108 mg, 0.15 mmol) to afford title compound (133 mg, 0.32 mmol, 43% yield).

LC-MS (ESI): m/z (M+1): 420.2 (Method 1)

### Intermediate 50: 6-(5-chloro-2-fluorophenyl)-3-(methylsulfanyl)pyridazin-4-amine

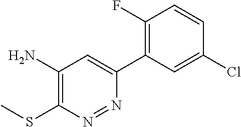 [SMILES: CSc1nnc(-c2cc(Cl)ccc2F)cc1N]

Intermediate 50 was prepared following the procedure used for the synthesis of Intermediate 9, starting from 6-(5-chloro-2-fluorophenyl)-N-[(2,4-dimethoxyphenyl)methyl]-3-(methylsulfanyl)pyridazin-4-amine (Intermediate 49, 133 mg, 0.32 mmol) to afford title compound (98 mg, recovery assumed quantitative).

LC-MS (ESI): m/z (M+1): 270.1 (Method 1)

### Intermediate 51: N-(4-{[6-(5-chloro-2-fluorophenyl)-3-methanesulfinylpyridazin-4-yl]amino}pyridin-2-yl)prop-2-enamide

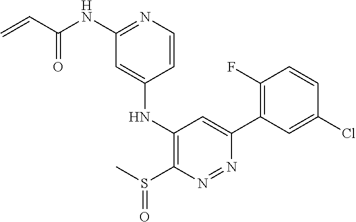 [SMILES: C=CC(=O)Nc1cc(Nc2cc(-c3cc(Cl)ccc3F)nnc2S(C)=O)ccn1]

A solution of N-(4-{[6-(5-chloro-2-fluorophenyl)-3-(methylsulfanyl)pyridazin-4-yl]amino}pyridin-2-yl)-3-(4-methylpiperazin-1-yl)propanamide (Example 16, 97 mg, 0.19 mmol) in MeOH (2.8 mL) and H₂O (0.94 mL) was treated with Oxone® (173 mg, 0.56 mmol) and stirred at RT for 16 hrs. Oxone® (29 mg, 0.09 mmol) was added and the mixture stirred for 2 hrs. Oxone (29 mg, 0.09 mmol) was added again and the mixture stirred for 2 hrs. Oxone® (11.5 mg, 0.04 mmol) was added again. Concomitant retro-Michael reaction occurred during the oxidation. The mixture was diluted with water and extracted with DCM. The organic phase was dried with Na₂SO₄, filtered, and concentrated under reduced pressure. The crude product was purified by flash chromatography on Biotage silica cartridge (from DCM to 10% MeOH) to afford the title compound (50 mg, 0.12 mmol, 62% yield. LC-MS (ESI): m/z (M+1): 432.2 (Method 2)

### Intermediate 52: N-(4-{[6-(5-chloro-2-fluorophenyl)-3-methanesulfonylpyridazin-4-yl]amino}pyridin-2-yl)prop-2-enamide

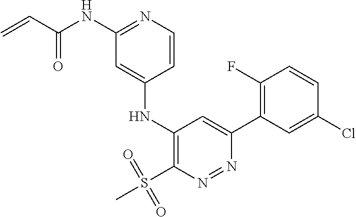 [SMILES: C=CC(=O)Nc1cc(Nc2cc(-c3cc(Cl)ccc3F)nnc2S(C)(=O)=O)ccn1]

A mixture of N-(4-{[6-(5-chloro-2-fluorophenyl)-3-methanesulfinylpyridazin-4-yl]amino}pyridin-2-yl)prop-2-enamide (Intermediate 51, 31 mg, 0.07 mmol) in MeOH (0.54 mL) and H₂O (0.18 mL) was treated with Oxone® (44 mg, 0.14 mmol) and stirred at RT for 16 hrs. The mixture was diluted with H₂O and extracted with EtOAc. The organic phase was dried with Na₂SO₄, filtered and concentrated under reduced pressure to give the title compound (33 mg, recovery assumed quantitative).

LC-MS (ESI): m/z (M+1): 448.2 (Method 2)

### Intermediate 53: 6-(5-chloro-2-fluorophenyl)-N-[(2,4-dimethoxyphenyl)methyl]-3-methanesulfinylpyridazin-4-amine

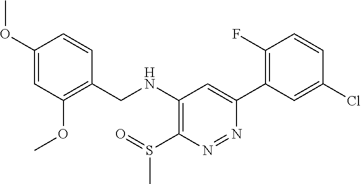 [SMILES: COc1ccc(CNc2cc(-c3cc(Cl)ccc3F)nnc2S(C)=O)c(OC)c1]

A solution of 6-(5-chloro-2-fluorophenyl)-N-[(2,4-dimethoxyphenyl)methyl]-3-(methylsulfanyl)pyridazin-4-amine (Intermediate 49, 269 mg, 0.64 mmol) in MeOH (4.67 mL) and H₂O (1.56 mL) was treated with Oxone® (256 mg, 0.83 mmol) and stirred at RT for 2 hrs. The mixture was diluted with water, extracted with EtOAc and washed with saturated NaHCO₃ aqueous solution. The organic phase was dried over Na₂SO₄, filtered and concentrated under reduced pressure. The crude was purified by flash chromatography on Biotage silica cartridge (from c-Hex to 30% EtOAc) to afford the title compound (242 mg, 0.55 mmol, 87% yield). LC-MS (ESI): m/z (M+1): 436.2 (Method 1)

### Intermediate 54: tert-butyl N-{[6-(5-chloro-2-fluorophenyl)-4-{[(2,4-dimethoxyphenyl)methyl]amino}pyridazin-3-yl](methyl)oxo-λ⁶-sulfanylidene}carbamate

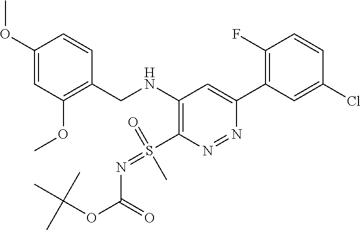 [SMILES: COc1ccc(CNc2cc(-c3cc(Cl)ccc3F)nnc2S(C)(=O)=NC(=O)OC(C)(C)C)c(OC)c1]

A solution of 6-(5-chloro-2-fluorophenyl)-N-[(2,4-dimethoxyphenyl)methyl]-3-methanesulfinylpyridazin-4-amine (Intermediate 53, 242 mg, 0.56 mmol), magnesium oxide (89 mg, 2.22 mmol), Rhodium(II) acetate dimer (12 mg, 0.03 mmol), carbamic acid tert-butyl ester (97 mg, 0.83 mmol) and (diacetoxyiodo)benzene (268 mg, 0.83 mmol) was stirred in anhydrous DCM (5.6 mL), at 40° C., for 16 hrs. The mixture was diluted with water and extracted with DCM. The organic phase was dried over Na₂SO₄, filtered and concentrated under reduced pressure. The crude product by flash chromatography on Biotage silica cartridge (from c-Hex to 30% EtOAc) to give the title compound (77 mg, 0.14 mmol, 25% yield). LC-MS (ESI): m/z (M+1): 551.2 (Method 1)

### Intermediate 55: tert-butyl N-{[4-amino-6-(5-chloro-2-fluorophenyl)pyridazin-3-yl](methyl)oxo-λ⁶-sulfanylidene}carbamate

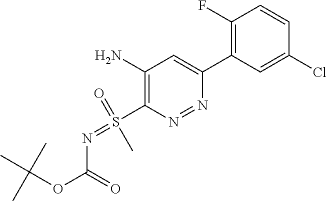 [SMILES: CC(C)(C)OC(=O)N=S(C)(=O)c1nnc(-c2cc(Cl)ccc2F)cc1N]

A solution of tert-butyl N-{[6-(5-chloro-2-fluorophenyl)-4-{[(2,4-dimethoxyphenyl)methyl]amino}pyridazin-3-yl](methyl)oxo-λ⁶-sulfanylidene}carbamate (Intermediate 54, 77 mg, 0.14 mmol) in MeCN (2.2 mL) and H₂O (0.2 mL) was treated with ammonium cerium(IV) nitrate (230 mg, 0.42 mmol) and the mixture stirred at RT for 1 h. The reaction was diluted with water and extracted with EtOAc. The organic phase was dried over Na₂SO₄, filtered and concentrated under reduced pressure. The crude product was purified by flash chromatography on Biotage silica cartridge (from c-Hex to 30% EtOAc) to give the title compound (42 mg, 0.10 mmol, 75% yield). LC-MS (ESI): m/z (M+1): 401.1 (Method 1)

### Intermediate 56: tert-butyl N-{[6-(5-chloro-2-fluorophenyl)-4-({2-[3-(4-methylpiperazin-1-yl)propanamido]pyridin-4-yl}amino)pyridazin-3-yl](methyl)oxo-λ⁶-sulfanylidene}carbamate

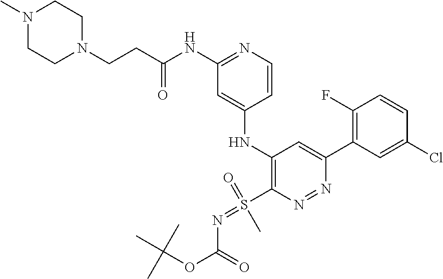

Intermediate 56 was prepared following the procedure used for the synthesis of Intermediate 47, starting from Intermediate 55 (17 mg, 0.04 mmol) and Intermediate 2 (15 mg, 0.05 mmol) to afford title compound (28 mg, recovery assumed quantitative).

LC-MS (ESI): m/z (M+1): 647.3 (Method 2)

### Intermediate 57: tert-butyl 4-{2-[(4-bromopyridin-2-yl)carbamoyl]ethyl}piperazine-1-carboxylate

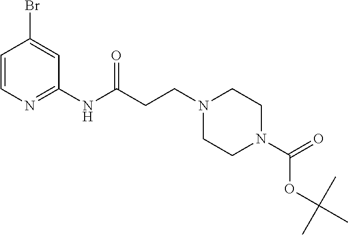 [SMILES: CC(C)(C)OC(=O)N1CCN(CCC(=O)Nc2cc(Br)ccn2)CC1]

Intermediate 57 was prepared following the procedure used for the synthesis of Intermediate 2, starting from N-(4-bromopyridin-2-yl)prop-2-enamide (Intermediate 1, 350 mg, 1.54 mmol) and 1-piperazinecarboxylic acid tert-butyl ester (373 mg, 2.0 mmol) to afford title compound (658 mg, recovery assumed quantitative).

LC-MS (ESI): m/z (M+1): 413.2 (Method 1)

### Intermediate 58: N-(4-bromopyridin-2-yl)-3-(piperazin-1-yl)propanamide

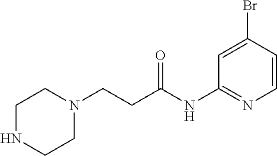 [SMILES: O=C(CCN1CCNCC1)Nc1cc(Br)ccn1]

Intermediate 58 was prepared following the procedure used for the synthesis of Intermediate 40, starting from Intermediate 57 (650 mg, 1.41 mmol) to afford title compound (488 mg, recovery assumed quantitative).

LC-MS (ESI): m/z (M+1): 313.5 (Method 2)

### Intermediate 59: tert-butyl N-[2-(4-{2-[(4-bromopyridin-2-yl)carbamoyl]ethyl}piperazin-1-yl)ethyl]carbamate

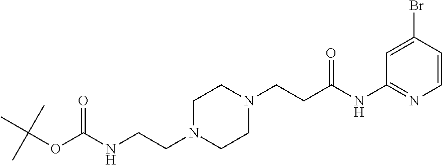 [SMILES: CC(C)(C)OC(=O)NCCN1CCN(CCC(=O)Nc2cc(Br)ccn2)CC1]

To a solution of N-(4-bromopyridin-2-yl)-3-(piperazin-1-yl)propanamide (Intermediate 58, 140 mg, 0.45 mmol) in MeOH (1.5 mL), acetic acid (0.08 mL, 1.34 mmol) and tert-butyl N-(2-oxoethyl)carbamate (107 mg, 0.67 mmol) were added. This mixture was stirred at RT for 30 min, before adding sodium cyanoborohydride (42 mg, 0.67 mmol). After 30 min further tert-butyl N-(2-oxoethyl)carbamate (53 mg, 0.34 mmol) and sodium cyanoborohydride (21 mg, 0.34 mmol) were added again. After 30 min, volatiles were removed under vacuum. The crude material was purified by flash chromatography on Biotage silica NH cartridge (from c-Hex to 30% EtOAc) to give the title compound (79 mg, 0.17 mmol, 39% yield).

LC-MS (ESI): m/z (M+1): 456.4 (Method 2)

### Intermediate 60: tert-butyl N-[2-(4-{2-[(4-{[6-(5-chloro-2-fluorophenyl)-3-(methylsulfanyl)pyridazin-4-yl]amino}pyridin-2-yl)carbamoyl]ethyl}piperazin-1-yl)ethyl]carbamate

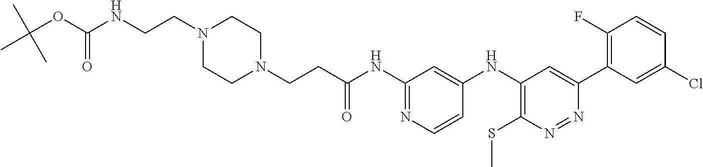 [SMILES: CSc1nnc(-c2cc(Cl)ccc2F)cc1Nc1ccnc(NC(=O)CCN2CCN(CCNC(=O)OC(C)(C)C)CC2)c1]

Intermediate 60 was prepared following the procedure used for the synthesis of Intermediate 47, starting from Intermediate 50 (42 mg, 0.16 mmol) and Intermediate 59 (78 mg, 0.17 mmol) to afford title compound (115 mg, recovery assumed quantitative).

LC-MS (ESI): m/z (M+1): 645.4 (Method 2)

### Intermediate 61: 2-[(6-chloro-4-{[(2,4-dimethoxyphenyl)methyl]amino}pyridazin-3-yl)sulfanyl]ethan-1-ol

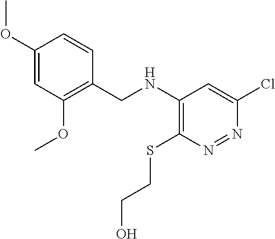 [SMILES: COc1ccc(CNc2cc(Cl)nnc2SCCO)c(OC)c1]

To a solution of 2-mercaptoethanol (0.13 mL, 1.78 mmol) in DMF (8 mL), NaH 60% dispersion in oil (71.3 mg, 1.78 mmol) was added and the mixture was stirred at RT for 1 h. 3,6-dichloro-N-[(2,4-dimethoxyphenyl)methyl]pyridazin-4-amine (Intermediate 6, 400 mg, 1.27 mmol) dissolved in DMF (2 mL) was added and the mixture was stirred at RT overnight. The mixture was poured into saturated NaHCO₃ aqueous solution and extracted with EtOAc. The organic phase was separated, filtered through a hydrophobic phase separator, and concentrated at reduced pressure. The crude was washed with a small amount of DCM and filtered to obtain a first crop as a solid. The filtrate was concentrated at reduced pressure and it was purified by flash chromatography on Biotage silica cartridge (from c-Hex to 100% EtOAc) The product so obtained was mixed with the first batch to afford the title compound (257 mg, 0.29 mmol, 57% yield).

LC-MS (ESI): m/z (M+1): 356.1 (Method 1)

### Intermediate 62: 3-({2-[(tert-butyldimethylsilyl)oxy]ethyl}sulfanyl)-6-chloro-N-[(2,4-dimethoxyphenyl)methyl]pyridazin-4-amine

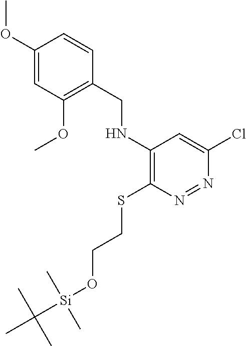 [SMILES: COc1ccc(CNc2cc(Cl)nnc2SCCO[Si](C)(C)C(C)(C)C)c(OC)c1]

To a solution of 2-[(6-chloro-4-{[(2,4-dimethoxyphenyl)methyl]amino}pyridazin-3-yl)sulfanyl]ethan-1-ol (Intermediate 61, 257 mg, 0.29 mmol) in DCM (7 mL), TEA (0.25 mL, 1.81 mmol) was added followed by the addition of tert-butyl-chloro-dimethylsilane (218 mg, 1.44 mmol). The mixture was stirred at RT overnight, then was diluted with DCM and washed with saturated NH₄Cl aqueous solution. The organic phase was separated, filtered through a hydrophobic phase separator and concentrated at reduced pressure. The crude was purified by flash chromatography on Biotage silica cartridge (from c-Hex to 25% EtOAc). Evaporation of proper fractions provided the title compound (227 mg, 0.48 mmol, 67% yield).

LC-MS (ESI): m/z (M+1): 470.2 (Method 1)

### Intermediate 63: 3-({2-[(tert-butyldimethylsilyl)oxy]ethyl}sulfanyl)-6-(5-chloro-2-fluorophenyl)-N-[(2,4-dimethoxyphenyl)methyl]pyridazin-4-amine

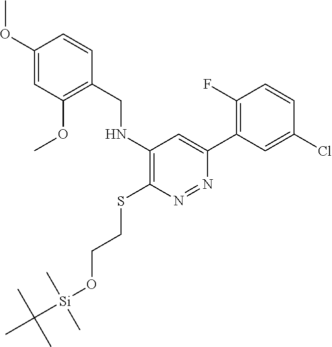 [SMILES: COc1ccc(CNc2cc(-c3cc(Cl)ccc3F)nnc2SCCO[Si](C)(C)C(C)(C)C)c(OC)c1]

Intermediate 63 was prepared following the procedure used for the synthesis of Intermediate 8, starting from Intermediate 62 (227 mg, 0.48 mmol) and 5-chloro-2-fluorobenzeneboronic acid (126 mg, 0.72 mmol) in presence of Pd(dppf)Cl₂ (71 mg, 0.10 mmol) to afford title compound (158 mg, 0.28 mmol, 58% yield).

LC-MS (ESI): m/z (M+1): 564.1 (Method 1)

### Intermediate 64: 2-{[4-amino-6-(5-chloro-2-fluorophenyl)pyridazin-3-yl]sulfanyl}ethan-1-ol

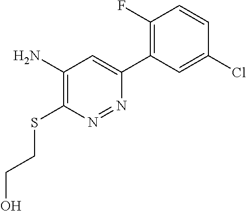 [SMILES: Nc1cc(-c2cc(Cl)ccc2F)nnc1SCCO]

To a solution of Intermediate 63 (158 mg, 0.28 mmol) in MeCN (2.7 mL) and buffer solution (pH=9, 0.6 mL), ammonium cerium (IV) nitrate (181 mg, 0.70 mmol) was added and the mixture was stirred at RT for 30 minutes. The mixture was diluted with EtOAc and washed with saturated NaHCO₃ aqueous solution. The organic phase was separated, filtered through a hydrophobic phase separator, and concentrated at reduced pressure. The crude was purified by flash chromatography on Biotage silica cartridge (from DCM to 50% EtOAc). Evaporation of opportune fractions provided the title compound (63 mg, 0.21 mmol, 75% yield). LC-MS (ESI): m/z (M+1): 300.0 (Method 1)

### Intermediate 65: 2-[(tert-butyldimethylsilyl)oxy]ethane-1-thiol

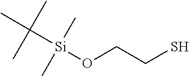

To a stirred solution of 2-mercaptoethanol (1.8 mL, 25.6 mmol) and imidazole (3.49 g, 51.2 mmol) in DCM (20 mL), tert-butyl-chloro-dimethylsilane (4.24 g, 28.2 mmol) was added. The reaction was stirred at RT overnight. Water was added, phases were separated, and the organic was washed with more water. The organic phase was filtered through a phase separator and evaporated under vacuum. The crude material was purified by flash chromatography on Biotage silica cartridge (from c-Hex to 20% EtOAc), to give the title compound (3.70 g, 19.2 mmol, 76% yield).

¹H NMR (400 MHz, Chloroform-d) δ ppm 3.74 (t, J=6.38 Hz, 2H) 2.64 (dt, J=8.31, 6.41 Hz, 2H) 1.49-1.58 (m, 1H) 0.92 (s, 9H) 0.09 (s, 6H).

### Intermediate 66: 3-({2-[(tert-butyldimethylsilyl)oxy]ethyl}sulfanyl)-6-chloropyridazin-4-amine

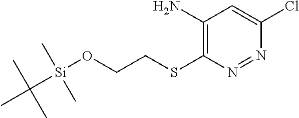 [SMILES: CC(C)(C)[Si](C)(C)OCCSc1nnc(Cl)cc1N]

Intermediate 66 was prepared following the procedure used for the synthesis of Intermediate 61, starting from 3,6-dichloropyridazin-4-amine (1.7 g, 10.4 mmol) and Intermediate 65 (2.99 g, 15.6 mmol) to afford title compound (2.56 g, 8.01 mmol, 77% yield). LC-MS (ESI): m/z (M+1): 320.9 (Method 1)

### Intermediate 67: 3-({2-[(tert-butyldimethylsilyl)oxy]ethyl}sulfanyl)-6-(5-chloro-2-fluorophenyl)pyridazin-4-amine

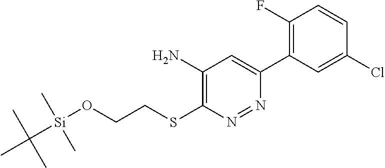 [SMILES: CC(C)(C)[Si](C)(C)OCCSc1nnc(-c2cc(Cl)ccc2F)cc1N]

### Method A

Intermediate 67 was prepared following the procedure used for the synthesis of Intermediate 62, starting from 2-{[4-amino-6-(5-chloro-2-fluorophenyl)pyridazin-3-yl]sulfanyl}ethan-1-ol (Intermediate 64, 63 mg, 0.21 mmol) to afford title compound (80 mg, 0.19 mmol, 92% yield).

### Method B

Intermediate 67 was also prepared following the procedure used for the synthesis of Intermediate 8, starting from Intermediate 66 (2.96 g, 9.25 mmol) and 5-chloro-2-fluorobenzeneboronic acid (2.42 g, 13.88 mmol) in presence of Pd(dppf)Cl₂ (1.35 g, 1.85 mmol) to afford title compound (2.3 g, 5.56 mmol, 60% yield).

LC-MS (ESI): m/z (M+1): 414.1 (Method 1)

### Intermediate 68: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)propanamide

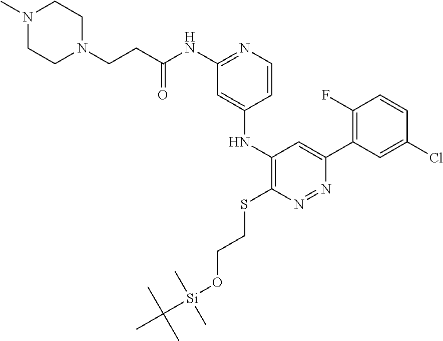 [SMILES: CN1CCN(CCC(=O)Nc2cc(Nc3cc(-c4cc(Cl)ccc4F)nnc3SCCO[Si](C)(C)C(C)(C)C)ccn2)CC1]

Intermediate 68 was prepared following the procedure used for the synthesis of Intermediate 47, starting from Intermediate 67 (80 mg, 0.19 mmol) and Intermediate 2 (70 mg, 0.21 mmol) to afford title compound (134 mg, recovery assumed quantitative).

LC-MS (ESI): m/z (M+1): 660.2 (Method 2)

### Intermediate 69: N-(4-bromopyridin-2-yl)-4-chlorobutanamide

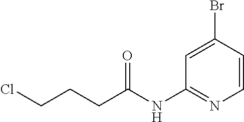 [SMILES: O=C(CCCCl)Nc1cc(Br)ccn1]

4-chlorobutanoyl chloride (0.71 mL, 6.36 mmol) was added dropwise to a stirred solution of 4-bromopyridin-2-amine (1 g, 5.78 mmol), TEA (2.42 mL, 17.3 mmol) in dry DCM (30 mL) at 0° C. under N₂. The resulting mixture was stirred at RT overnight. Further 4-chlorobutanoyl chloride (0.3 ml, 2.6 mmol) was added at 0° C. The mixture was stirred at RT for 3 hrs. The mixture was then diluted with more DCM and washed with brine. The organic phase was separated, dried over Na₂SO₄, and filtered. The solvent was evaporated to give a crude oil which was purified by flash chromatography on Biotage silica cartridge (from c-Hex to 30% EtOAc) to afford the title compound (1.29 g, 4.65 mmol, 80% yield). LC-MS (ESI): m/z (M+1): 276.9(Method 1)

### Intermediate 70: N-(4-bromopyridin-2-yl)-4-(4-methylpiperazin-1-yl)butanamide

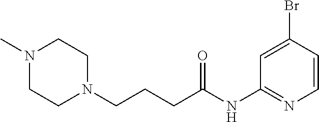 [SMILES: CN1CCN(CCCC(=O)Nc2cc(Br)ccn2)CC1]

A solution of N-(4-bromopyridin-2-yl)-4-chlorobutanamide (Intermediate 69, 500 mg, 1.8 mmol), 1-methylpiperazine (2.0 mL, 18.0 mmol) and TEA (0.75 mL, 5.4 mmol) in THE (7.2 mL) was heated at 70° C. for 24 hrs. Volatiles were removed under vacuum. The residue was taken up with EtOAc and washed with saturated NaHCO₃ aqueous solution. The organic phase was washed with brine, separated, filtered through a phase separator and evaporated under vacuum. The crude material was purified by flash chromatography on Biotage silica NH cartridge (from c-Hex to 40% EtOAc) to give the title compound (475 mg, 1.39 mmol, 77% yield). LC-MS (ESI): m/z (M+1): 341.6 (Method 2)

### Intermediate 71: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-4-(4-methylpiperazin-1-yl)butanamide

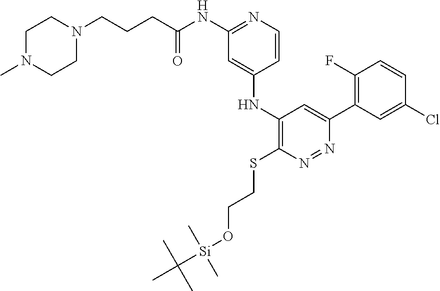 [SMILES: CN1CCN(CCCC(=O)Nc2cc(Nc3cc(-c4cc(Cl)ccc4F)nnc3SCCO[Si](C)(C)C(C)(C)C)ccn2)CC1]

Intermediate 71 was prepared following the procedure used for the synthesis of Intermediate 47, starting from Intermediate 67 (80 mg, 0.19 mmol) and Intermediate 70 (73 mg, 0.21 mmol) to afford title compound (98 mg, 0.14 mmol, 75% yield).

LC-MS (ESI): m/z (M+1): 674.3 (Method 2)

### Intermediate 72: N-(4-bromopyridin-2-yl)-2-{6-methyl-2,6-diazaspiro[3.3]heptan-2-yl}acetamide

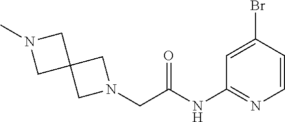 [SMILES: CN1CC2(C1)CN(CC(=O)Nc1cc(Br)ccn1)C2]

TEA (0.87 mL, 6.25 mmol) was added to a stirred solution of 2-methyl-2,6-diazaspiro[3.3]heptane dihydrochloride (386 mg, 2.08 mmol) in dry MeCN (8 mL) at RT under N₂. After 10 minutes the reaction was cooled with an ice bath, and N-(4-bromopyridin-2-yl)-2-chloroacetamide (Intermediate 33, 350 mg, 1.39 mmol) was added followed by catalytic amount of potassium iodide. The reaction mixture was allowed to reach RT and stirred for 2 hrs at this temperature. Water and EtOAc were added, the organic phases were separated and the aqueous phase was extracted with EtOAc. The combined organics were dried over Na₂SO₄ and filtered. The solvent was evaporated and the crude material was purified by flash chromatography on Biotage silica NH cartridge (from c-Hex to 45% EtOAc) to afford the title compound (330 mg, 1.01 mmol, 73% yield). LC-MS (ESI): m/z (M+1): 325.0 (Method 2)

### Intermediate 73: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-{6-methyl-2,6-diazaspiro[3.3]heptan-2-yl}acetamide

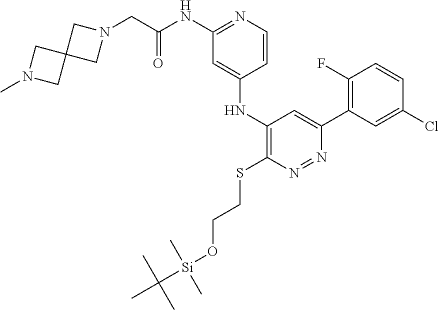 [SMILES: CN1CC2(C1)CN(CC(=O)Nc1cc(Nc3cc(-c4cc(Cl)ccc4F)nnc3SCCO[Si](C)(C)C(C)(C)C)ccn1)C2]

Intermediate 73 was prepared following the procedure used for the synthesis of Intermediate 47, starting from Intermediate 67 (210 mg, 0.51 mmol) and Intermediate 72 (318 mg, 0.56 mmol), to afford title compound (224 mg, 0.34 mmol, 67% yield).

LC-MS (ESI): m/z (M+1): 658.3 (Method 2)

### Intermediate 74: 2-methyl-2,5-diazabicyclo[2.2.1]heptane dihydrochloride

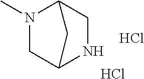

2-methyl-2,5-diazabicyclo[2.2.1]heptane dihydrobromide (1 g, 3.65 mmol) was charged in a SCX and eluted with 1 N NH₃ in MeOH. The fractions were concentrated under reduced pressure and then treated with HCl (4N solution in 1,4-dioxane) (2.74 mL, 10.95 mmol). The mixture was concentrated under reduced pressure to afford the title compound (330 mg, 1.78 mmol, 49% yield). LC-MS (ESI): m/z (M+1): 113.2 (Method 2)

### Intermediate 75: N-(4-bromopyridin-2-yl)-2-{5-methyl-2,5-diazabicyclo[2.2.1]heptan-2-yl}acetamide

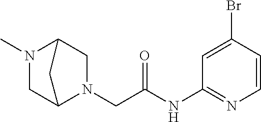 [SMILES: CN1CC2CC1CN2CC(=O)Nc1cc(Br)ccn1]

Intermediate 75 was prepared following the procedure used for the synthesis of Intermediate 39, starting from Intermediate 33 (200 mg, 0.79 mmol) and Intermediate 74 (220 mg, 1.19 mmol) to afford title compound (160 mg, 0.49 mmol, 62% yield).

LC-MS (ESI): m/z (M+1): 325.1 (Method 2)

### Intermediate 76: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-{5-methyl-2,5-diazabicyclo[2.2.1]heptan-2-yl}acetamide

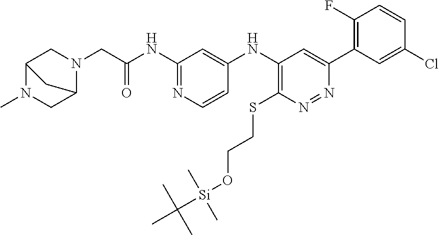 [SMILES: CN1CC2CC1CN2CC(=O)Nc1cc(Nc2cc(-c3cc(Cl)ccc3F)nnc2SCCO[Si](C)(C)C(C)(C)C)ccn1]

Intermediate 76 was prepared following the procedure used for the synthesis of Intermediate 47, starting from Intermediate 67 (180 mg, 0.43 mmol) and Intermediate 75 (155 mg, 0.48 mmol) to afford title compound (160 mg, 0.24 mmol, 56% yield).

LC-MS (ESI): m/z (M+1): 658.3 (Method 2)

### Intermediate 77: N-(4-bromopyridin-2-yl)-2,2,2-trichloroacetamide

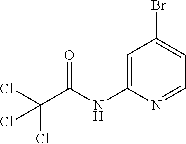 [SMILES: O=C(Nc1cc(Br)ccn1)C(Cl)(Cl)Cl]

A solution of 4-bromo-2-pyridinamine (780 mg, 4.51 mmol) and TEA (0.69 mL, 4.96 mmol) in THE (23 mL) was treated with 2,2,2-trichloroacetyl chloride (0.48 mL, 4.28 mmol) at 0° C. The mixture was stirred at the same temperature for 10 minutes and then at RT for 4 hrs. The mixture was cooled to 0° C. and carefully quenched with water and then saturated NaHCO₃ solution. The mixture was extracted with EtOAc, dried with Na₂SO₄, filtered and concentrated under reduced pressure. The crude product was purified by flash chromatography on Biotage silica NH (from c-Hex to 100% EtOAc) to afford the title compound (1.10 g, 3.45 mmol, 77% yield).

LC-MS (ESI): m/z (M+1): 316.8 (Method 1)

### Intermediate 78: tert-butyl 2-methyl-2,8-diazaspiro[4.5]decane-8-carboxylate

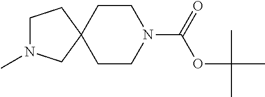 [SMILES: CN1CCC2(CCN(C(=O)OC(C)(C)C)CC2)C1]

Intermediate 78 was prepared following the procedure used for the synthesis of Intermediate 31, starting from tert-butyl 2,8-diazaspiro[4.5]decane-8-carboxylate (3.16 g, 13.15 mmol) and formaldehyde 37% w/w in water (4.95 mL, 65.8 mmol) to afford title compound (1.37 g, 5.41 mmol, 41% yield). LC-MS (ESI): m/z (M+1): 255.4 (Method 2) Intermediate 79: 2-methyl-2,8-diazaspiro[4.5]decane dihydrochloride

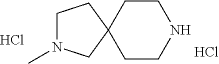

Intermediate 79 was prepared following the procedure used for the synthesis of Intermediate 32, starting from tert-butyl 2-methyl-2,8-diazaspiro[4.5]decane-8-carboxylate (Intermediate 78, 1.37 g, 5.4 mmol) to afford title compound (912 mg, 4 mmol, 74% yield).

¹H NMR (500 MHz, Methanol-d₄) δ ppm 3.76 (ddd, J=11.7, 7.8, 3.9 Hz, 1H), 3.69 (d, J=12.1 Hz, 1H), 3.18-3.29 (m, 5H), 3.04 (d, J=12.1 Hz, 1H), 2.97 (s, 3H), 2.15-2.26 (m, 1H), 1.87-2.10 (m, 5H).

### Intermediate 80: N-(4-bromopyridin-2-yl)-2-methyl-2,8-diazaspiro[4.5]decane-8-carboxamide

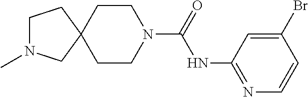 [SMILES: CN1CCC2(CCN(C(=O)Nc3cc(Br)ccn3)CC2)C1]

A mixture of N-(4-bromopyridin-2-yl)-2,2,2-trichloroacetamide (Intermediate 77, 200 mg, 0.63 mmol) and 2-methyl-2,8-diazaspiro[4.5]decane dihydrochloride (Intermediate 79, 157 mg, 0.69 mmol) in DMSO (4.2 mL), and Na₂CO₃ (233 mg, 2.2 mmol) was stirred at 100° C. for 2.5 hrs. The mixture was treated with saturated NaHCO₃ solution and extracted with DCM. The organic phase was dried over Na₂SO₄, filtered, and concentrated under reduced pressure. The crude product was purified by flash chromatography on Biotage silica NH (from EtOAc to 10% MeOH), then further purified by reverse flash chromatography on Biotage C18 cartridge (from H₂O+0.1% HCOOH to 20% MeCN+0.1% HCOOH), and eluted through a PL-HCO₃ cartridge using MeOH to afford the title compound (103 mg, 0.29 mmol, 46% yield).

LC-MS (ESI): m/z (M+1): 353.1 (Method 1)

### Intermediate 81: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-methyl-2,8-diazaspiro[4.5]decane-8-carboxamide

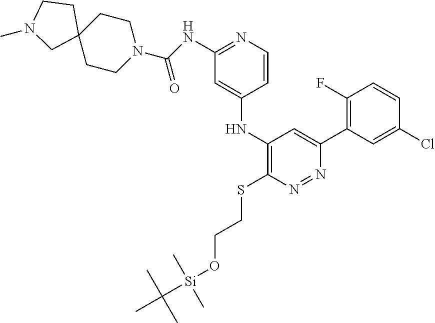 [SMILES: CN1CCC2(CCN(C(=O)Nc3cc(Nc4cc(-c5cc(Cl)ccc5F)nnc4SCCO[Si](C)(C)C(C)(C)C)ccn3)CC2)C1]

Intermediate 81 was prepared following the procedure used for the synthesis of Intermediate 47, starting from Intermediate 67 (100 mg, 0.24 mmol) and Intermediate 80 (95 mg, 0.27 mmol) to afford title compound (130 mg, 0.19 mmol, 78% yield).

LC-MS (ESI): m/z (M+1): 686.3 (Method 2)

### Intermediate 82: N-(4-bromopyridin-2-yl)-2-(4-methyl-1,4-diazepan-1-yl)acetamide

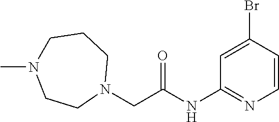 [SMILES: CN1CCCN(CC(=O)Nc2cc(Br)ccn2)CC1]

N-(4-bromopyridin-2-yl)-2-chloroacetamide (Intermediate 33, 300 mg, 1.20 mmol) was added to a stirred solution of 1-methyl-1,4-diazepane (275 mg, 2.40 mmol) in dry DMF (4.55 mL) at RT. After 3 hrs the mixture was treated with H₂O and extracted with EtOAc. Organic layer was separated, washed with water, dried over Na₂SO₄, filtered and evaporated. The crude material was purified by flash chromatography on Biotage silica NH (from c-Hex to 40% EtOAc) to afford (209 mg, 0.64 mmol, 53% yield).

LC-MS (ESI): m/z (M+1): 327.4 (Method 2)

### Intermediate 83: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-(4-methyl-1,4-diazepan-1-yl)acetamide

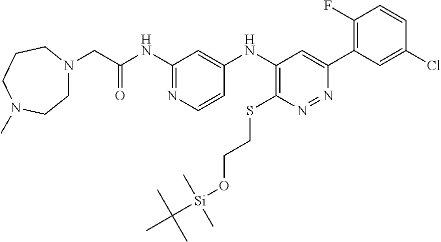 [SMILES: CN1CCCN(CC(=O)Nc2cc(Nc3cc(-c4cc(Cl)ccc4F)nnc3SCCO[Si](C)(C)C(C)(C)C)ccn2)CC1]

Intermediate 83 was prepared following the procedure used for the synthesis of Intermediate 47, starting from Intermediate 67 (100 mg, 0.24 mmol) and Intermediate 82 (87 mg, 0.27 mmol) to afford title compound (213 mg, recovery assumed quantitative).

LC-MS (ESI): m/z (M+1): 660.3 (Method 2)

### Intermediate 84: 3-[(tert-butyldimethylsilyl)oxy]propane-1-thiol

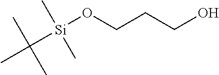

Intermediate 84 was also prepared following the procedure used for the synthesis of Intermediate 65, starting from 3-mercapto-1-propanol (1 g, 10.85 mmol) to afford title compound (1.8 g, 8.72 mmol, 80% yield).

¹H NMR (400 MHz, Chloroform-d) δ ppm 3.72 (t, J=5.94 Hz, 2H) 2.56-2.68 (m, 2H) 1.77-1.86 (m, 2H) 1.34 (t, J=7.92 Hz, 1H) 0.90 (s, 9H) 0.07 (s, 6H).

### Intermediate 85: 3-[3-[tert-butyl(dimethyl)silyl]oxypropylsulfanyl]-6-chloropyridazin-4-amine

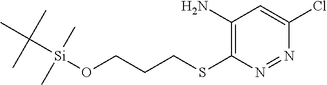 [SMILES: CC(C)(C)[Si](C)(C)OCCCSc1nnc(Cl)cc1N]

Intermediate 85 was prepared following the procedure used for the synthesis of Intermediate 61, starting from Intermediate 84 (1.8 g, 8.73 mmol) to afford title compound (1.62 g, 4.84 mmol, 83% yield). LC-MS (ESI): m/z (M+1): 334.2 (Method 1)

### Intermediate 86: 3-({3-[(tert-butyldimethylsilyl)oxy]propyl}sulfanyl)-6-(5-chloro-2-fluorophenyl)pyridazin-4-amine

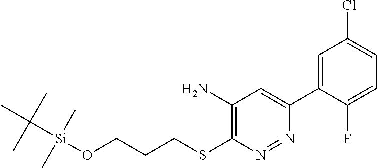 [SMILES: CC(C)(C)[Si](C)(C)OCCCSc1nnc(-c2cc(Cl)ccc2F)cc1N]

Intermediate 86 was prepared following the procedure used for the synthesis of Intermediate 8, starting from Intermediate 85 (488 mg, 1.86 mmol) and 5-chloro-2-fluorobenzeneboronic acid (382 mg, 2.19 mmol) in presence of Pd(dppf)Cl₂ (214 mg, 0.29 mmol) to afford title compound (300 mg, 0.70 mmol, 48% yield).

LC-MS (ESI): m/z (M+1): 428.2 (Method 1)

### Intermediate 87: N-(4-{[3-({3-[(tert-butyldimethylsilyl)oxy]propyl}sulfanyl)-6-(5-chloro-2-fluorophenyl)pyridazin-4-yl]amino}pyridin-2-yl)-3-(4-methylpiperazin-1-yl)propanamide

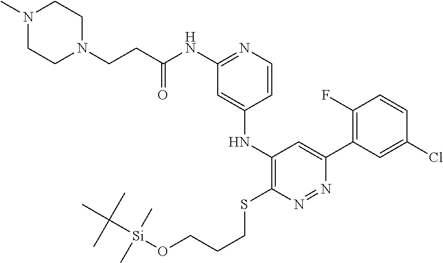 [SMILES: CN1CCN(CCC(=O)Nc2cc(Nc3cc(-c4cc(Cl)ccc4F)nnc3SCCCO[Si](C)(C)C(C)(C)C)ccn2)CC1]

Intermediate 87 was prepared following the procedure used for the synthesis of Intermediate 47, starting from Intermediate 86 (100 mg, 0.23 mmol) and Intermediate 2 (84 mg, 0.26 mmol) to afford title compound (150 mg, 0.022 mmol, 55% yield).

LC-MS (ESI): m/z (M+1): 674.5 (Method 2)

### Intermediate 88: 6-Chloro-N4-(2,4-dimethoxybenzyl)-N3-methyl pyridazine-3,4-diamine

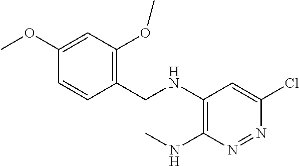 [SMILES: CNc1nnc(Cl)cc1NCc1ccc(OC)cc1OC]

To a solution of 3,6-dichloro-N-(2,4-dimethoxybenzyl)pyridazin-4-amine (Intermediate 6, 1.2 g, 3.82 mmol) in dry NMP (10 mL), TEA (0.532 mL, 3.82 mmol) and methanamine 33 wt. % in absolute ethanol (0.713 mL, 5.73 mmol) were added and the reaction was heated to 125° C. for 12 h. Then, the reaction was purified by reverse flash chromatography on Biotage C18 cartridge (from H₂O+0.1% HCOOH to 40% MeCN+0.1% HCOOH) to afford the title compound (0.6 g, 1.943 mmol, 51% yield) and 6-chloro-N4-(2,4-dimethoxybenzyl)-N3-methylpyridazine-3,4-diamine (0.6 g, 1.943 mmol, 51% yield) as a mixture of regioisomer. LC-MS (ESI): m/z (M+1): 308.9 (Method 1)

### Intermediate 89: 6-(2-chloro-5-fluorophenyl)-N4-(2,4-dimethoxybenzyl)-N3-methylpyridazine-3,4-diamine

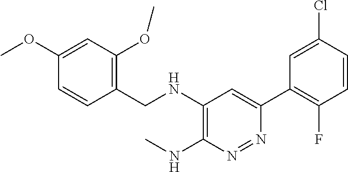 [SMILES: CNc1nnc(-c2cc(Cl)ccc2F)cc1NCc1ccc(OC)cc1OC]

Intermediate 89 was prepared following the procedure used for the synthesis of Intermediate 8, starting from Intermediate 88 (600 mg, 1.943 mmol) and using 5-chloro-2-fluorophenyl)boronic acid (1.016 g, 5.83 mmol). Purification by reverse flash chromatography on Biotage C18 cartridge (from H₂O+0.1% HCOOH to 40% MeCN +0.1% HCOOH) afforded the title compound (400 mg, 0.993 mmol, 51% yield).

LC-MS (ESI): m/z (M+1): 403.0 (Method 1)

### Intermediate 90: 6-(2-Chloro-5-fluorophenyl)-N3-methylpyridazine-3,4-diamine

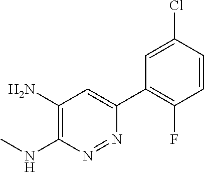 [SMILES: CNc1nnc(-c2cc(Cl)ccc2F)cc1N]

Intermediate 90 was prepared following the procedure used for the synthesis of Intermediate 9, starting from Intermediate 89 (0.160 g, 0.397 mmol) and using butanol (2 mL) as solvent. Purification by reverse flash chromatography on Biotage C18 cartridge (from H₂O+0.1% HCOOH to 30% MeCN+0.1% HCOOH) afforded the title compound (100 mg, 0.397 mmol, recovery assumed quantitative).

LC-MS (ESI): m/z (M+1): 252.9 (Method 1)

### Intermediate 91: 6-Chloro-N4-(2,4-dimethoxybenzyl)-N3,N3-dimethylpyridazine-3,4-diamine

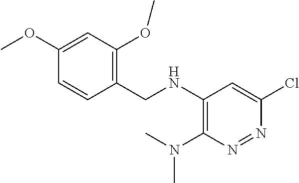 [SMILES: COc1ccc(CNc2cc(Cl)nnc2N(C)C)c(OC)c1]

Intermediate 91 was prepared following the procedure used for the synthesis of Intermediate 88, starting from Intermediate 6 (1.0 g, 3.18 mmol) and using dimethylamine 2.0 M in THF. Purification by reverse flash chromatography on Biotage C18 cartridge (from H₂O+0.1% HCOOH to 100% MeCN+0.1% HCOOH) afforded the title compound (172 mg, 0.533 mmol, 17% yield). LC-MS (ESI): m/z (M+1): 322.9 (Method 1)

### Intermediate 92: 6-(5-Chloro-2-fluorophenyl)-N4-(2,4-dimethoxybenzyl)-N3,N3-dimethylpyridazine-3,4-diamine

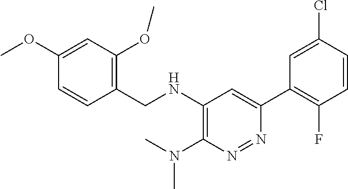 [SMILES: COc1ccc(CNc2cc(-c3cc(Cl)ccc3F)nnc2N(C)C)c(OC)c1]

Intermediate 92 was prepared followed the procedure used for the synthesis of Intermediate 8, starting from Intermediate 91 (170 mg, 0.527 mmol). Purification by reverse flash chromatography on Biotage C18 cartridge (from H₂O+0.1% HCOOH to 40% MeCN+0.1% HCOOH) afforded the title compound (120 mg, 0.288 mmol, 55% yield). LC-MS (ESI): m/z (M+1): 417.1 (Method 1)

### Intermediate 93: methyl 3,6-dichloropyridazine-4-carboxylate

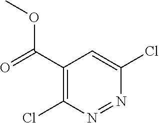

(Trimethylsilyl)diazomethane 2 M in hexane (7.12 mL, 14.2 mmol) was added dropwise to a stirred solution of 3,6-dichloro-4-pyridazinecarboxylic acid (2.5 g, 12.9 mmol) in MeOH (2.62 mL)/DCM (15 mL). The resulting solution was stirred at RT for 1 h, then further 5 mL of (trimethylsilyl)diazomethane 2 M in hexane were added dropwise and stirred for 1 h. Volatiles were removed under vacuum and the residue was purified by flash chromatography on Biotage silica (from c-Hex to 25% EtOAc) to afford the title compound (1.55 g, 7.49 mmol, 58% yield).

LC-MS (ESI): m/z (M+1): 207.1 (Method 1)

### Intermediate 94: methyl 6-chloro-3-(dimethylamino)pyridazine-4-carboxylate

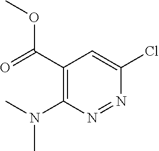

A mixture of methyl 3,6-dichloropyridazine-4-carboxylate (Intermediate 93, 138 mg, 0.67 mmol), DIPEA (0.17 mL, 1 mmol) and dimethylamine 2 M in THE (0.33 mL, 0.67 mmol) in dry 1,2-dimethoxyethane (2 mL) was heated at 80° C. for 18 hrs. Volatiles were removed under vacuum, and the crude material was purified by flash chromatography on Biotage silica (from c-Hex to 25% EtOAc) to afford the title compound (130 mg, 0.60 mmol, 90% yield). LC-MS (ESI): m/z (M+1): 216.2 (Method 1)

### Intermediate 95: methyl 6-(5-chloro-2-fluorophenyl)-3-(dimethylamino)pyridazine-4-carboxylate

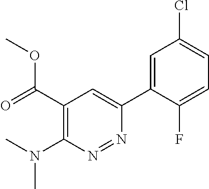 [SMILES: COC(=O)c1cc(-c2cc(Cl)ccc2F)nnc1N(C)C]

Intermediate 95 was prepared following the procedure used for the synthesis of Intermediate 8, starting from methyl 6-chloro-3-(dimethylamino)pyridazine-4-carboxylate (Intermediate 94, 125 mg, 0.58 mmol) and 5-chloro-2-fluorobenzeneboronic acid (202 mg, 1.16 mmol) in presence of Pd(dppfC₂ (85 mg, 0.12 mmol) to afford title compound (153 mg, 0.49 mmol, 85% yield). LC-MS (ESI): m/z (M+1): 310.1 (Method 1)

### Intermediate 96: 6-(5-chloro-2-fluorophenyl)-3-(dimethylamino)pyridazine-4-carboxylic acid

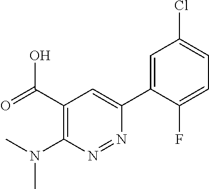 [SMILES: CN(C)c1nnc(-c2cc(Cl)ccc2F)cc1C(=O)O]

Lithium hydroxide hydrate (40.6 mg, 0.97 mmol) was added to a solution of methyl 6-(5-chloro-2-fluorophenyl)-3-(dimethylamino)pyridazine-4-carboxylate (Intermediate 95, 150 mg, 0.48 mmol) in H₂O (0.71 mL) and MeOH (4.29 mL). The resulting solution was stirred at RT overnight. Volatiles were removed under vacuum; the residue was diluted with EtOAc and saturated NH₄Cl solution was added until pH 7. A suspension was observed, volatiles were removed under vacuum and the residue was purified by reverse flash chromatography on Biotage C18 cartridge (from H₂O to 50% MeCN) to afford the title compound (140 mg, 0.47 mmol, 98% yield).

LC-MS (ESI): m/z (M+1): 296.1 (Method 1)

### Intermediate 97: 6-(5-chloro-2-fluorophenyl)-N3,N3-dimethylpyridazine-3,4-diamine

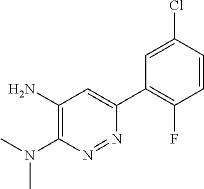 [SMILES: CN(C)c1nnc(-c2cc(Cl)ccc2F)cc1N]

### Method A

Intermediate 97 was prepared following the procedure used for the synthesis of Intermediate 9, starting from Intermediate 92 (0.120 g, 0.288 mmol). Purification by reverse flash chromatography on Biotage C18 cartridge (from H₂O+0.1% HCOOH to 30% MeCN+0.1% HCOOH) afforded the title compound (77 mg, 0.288 mmol, recovery assumed quantitative).

### Method B

TEA (79.2 μL, 0.57 mmol) and diphenylphosphoryl azide (112 μL, 0.52 mmol) were added to a solution 6-(5-chloro-2-fluorophenyl)-3-(dimethylamino)pyridazine-4-carboxylic acid (Intermediate 96, 140 mg, 0.47 mmol) in DMF (2 mL). The resulting solution was stirred at RT for 4 hrs, then H₂O (1.1 mL) was added, and the mixture was heated at 65° C. for 1.5 h. The mixture was concentrated under vacuum and purified by reverse flash chromatography on Biotage C18 cartridge (from H2O+0.1% HCOOH to 30% MeCN+0.1% HCOOH). Opportune fractions were evaporated, then the residue was charged on SCX, washed with MeOH, and eluted with 1N NH₃ in MeOH. Evaporation of basic fractions afforded the title compound (34 mg, 0.13 mmol, 27% yield).

LC-MS (ESI): m/z (M+1): 267.2 (Method 1)

### Intermediate 98: tert-butyl N-[(tert-butoxy)carbonyl]-N-(6-chloropyridazin-4-yl)carbamate

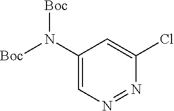 [SMILES: CC(C)(C)OC(=O)N(C(=O)OC(C)(C)C)c1cnnc(Cl)c1]

6-chloropyridazin-4-amine (2.0 g, 15.44 mmol) was dissolved in THE (80 mL), TEA (3.12 g, 30.88 mmol) and DMAP (0.09 g, 0.77 mmol) were added followed by di-tert-butyl dicarbonate (11.79 g, 54.03 mmol). The mixture was refluxed for 5 hrs. Then THE was evaporated and the residue partitioned between EtOAc and s.s. of NH₄Cl, the organic phase was dried and evaporated, the crude material was purified by flash chromatography on Biotage silica cartridge (from cHex to 30% EtOAc) to afford the title compound (3.96 g, 12.01 mmol, 78% yield). LC-MS (ESI): m/z (M+1): 330.1 (Method 1)

### Intermediate 99: tert-butyl N-[(tert-butoxy)carbonyl]-N-[6-(5-chloro-2-fluorophenyl)pyridazin-4-yl]carbamate

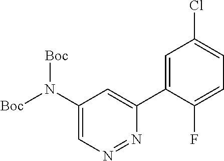 [SMILES: CC(C)(C)OC(=O)N(C(=O)OC(C)(C)C)c1cnnc(-c2cc(Cl)ccc2F)c1]

Intermediate 99 was prepared following the procedure used for the synthesis of Intermediate 8, starting from Intermediate 98 (1.0 g, 3.03 mmol). Purification by flash chromatography on Biotage silica cartridge (from cHex to 30% EtOAc) afforded the title compound (1.1 g, 2.6 mmol, 86% yield). LC-MS (ESI): m/z (M+1): 330.1 (Method 1)

### Intermediate 100: 6-(5-chloro-2-fluorophenyl)pyridazin-4-amine

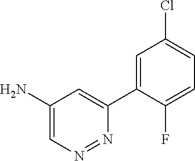 [SMILES: Nc1cnnc(-c2cc(Cl)ccc2F)c1]

Intermediate 99 (1.1 g, 2.6 mmol) was dissolved in DCM (10 mL) and TFA (3.0 mL, 39.18 mmol), the reaction solution was stirred for 5 hrs, then further 2 mL of TFA were added and the reaction was stirred overnight at RT. The day after volatiles were removed under vacuum, the residue was dissolved in MeOH and charged on SCX cartridge washing with MeOH and eluting with 1 N NH₃ in MeOH; basic fractions were collected to afford the title compound (530 mg, 2.37 mmol, 91% yield).

LC-MS (ESI): m/z (M+1): 224 (Method 2)

### Intermediate 101: 2-chloro-3-{[2-(trimethylsilyl)ethoxy]methyl}-3H-imidazo[4,5-b]pyridine

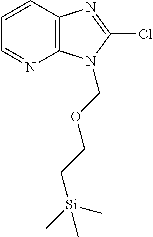 [SMILES: C[Si](C)(C)CCOCn1c(Cl)nc2cccnc21]

2-chloro-1H-imidazo[4,5-b]pyridine (200 mg, 1.3 mmol) was suspended in THE (8 mL) under N₂ and DIPEA (0.68 mL, 3.91 mmol) was added followed by 2-(chloromethoxy)ethyl-trimethylsilane (0.3 mL, 1.69 mmol). The reaction mixture was stirred at reflux for 4 hrs. Then it was allowed to reach RT, water and EtOAc were added, the product was extracted several times with EtOAc, organic phases were collected, dried and evaporated. The crude material by flash chromatography on Biotage silica cartridge (from cHex to 100% EtOAc) to afford the title compound (180 mg, 0.63 mmol 49% yield). LC-MS (ESI): m/z (M+1): 284. 2 (Method 1)

¹H NMR (400 MHz, Chloroform-d) δ ppm 8.40 (dd, J=4.8, 1.3 Hz, 1H), 7.99 (dd, J=8.0, 1.4 Hz, 1H), 7.29 (d, J=5.0 Hz, 1H), 5.71 (s, 2H), 3.61-3.72 (m, 2H), 0.91-1.00 (m, 2H), −0.05 (s, 9H).

### Intermediate 102: 6-(5-Chloro-2-fluorophenyl)-N-(3-{[2-(trimethylsilyl)ethoxy]methyl}-3H-imidazo[4,5-b]pyridin-2-yl)pyridazin-4-amine

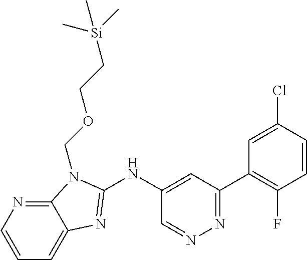 [SMILES: C[Si](C)(C)CCOCn1c(Nc2cnnc(-c3cc(Cl)ccc3F)c2)nc2cccnc21]

Intermediate 102 was prepared following the procedure used for the synthesis of Intermediate 18 starting from Intermediate 101 (113 mg, 0.40 mmol) and Intermediate 100 (70 mg, 0.31 mmol) to afford title compound (35 mg, 0.07 mmol, 24% yield).

LC-MS (ESI): m/z (M+1): 471.4 (Method 1)

### Intermediate 103: tert-butyl N-[(tert-butoxy)carbonyl]-N-(4-chloropyrimidin-2-yl)carbamate

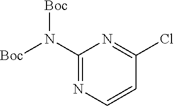 [SMILES: CC(C)(C)OC(=O)N(C(=O)OC(C)(C)C)c1nccc(Cl)n1]

Intermediate 103 was prepared following the procedure used for the synthesis of Intermediate 98, starting from 4-chloro-2-pyrimidinamine (200 mg, 1.54 mmol). Purification by flash chromatography on Biotage silica cartridge (from cHex to 20% EtOAc) afforded the title compound (500 mg, 1.52 mmol, 98% yield).

LC-MS (ESI): m/z (M+1): 330.3 (Method 1)

### Intermediate 104: tert-butyl N-[(tert-butoxy)carbonyl]-N-(4-{[6-(5-chloro-2-fluorophenyl)pyridazin-4-yl]amino}pyrimidin-2-yl)carbamate

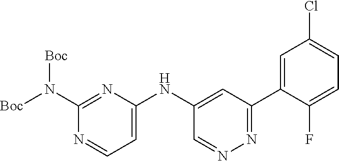 [SMILES: CC(C)(C)OC(=O)N(C(=O)OC(C)(C)C)c1nccc(Nc2cnnc(-c3cc(Cl)ccc3F)c2)n1]

Intermediate 104 was prepared following the procedure used for the synthesis of Intermediate 8 starting from Intermediate 103 (113 mg, 0.34 mmol) and Intermediate 100 (70 mg, 0.31 mmol) to afford the title compound (30 mg, 0.06 mmol, 18% yield).

LC-MS (ESI): m/z (M+1): 517.4 (Method 1)

### Intermediate 105: 6-chloro-N-[(2,4-dimethoxyphenyl)methyl]-3-(2-methoxyethoxy)pyridazin-4-amine

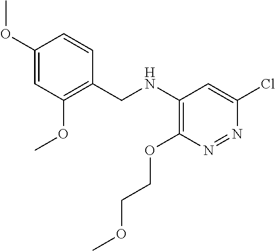 [SMILES: COCCOc1nnc(Cl)cc1NCc1ccc(OC)cc1OC]

Intermediate 105 was prepared following the procedure used for the synthesis of Intermediate 7, starting from 3,6-dichloro-N-[(2,4-dimethoxyphenyl)methyl]pyridazin-4-amine (Intermediate 6, 1 g, 3.18 mmol) and 2-methoxyethanol (0.33 mL, 4.14 mmol) to afford title compound (776 mg, 2.20 mmol, 69% yield).

LC-MS (ESI): m/z (M+1): 354.2 (Method 1)

### Intermediate 106: 6-(5-chloro-2-fluorophenyl)-N-[(2,4-dimethoxyphenyl)methyl]-3-(2-methoxyethoxy)pyridazin-4-amine

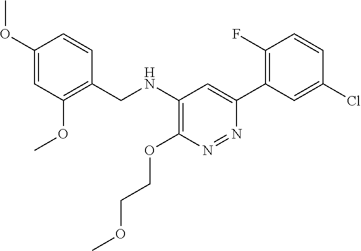 [SMILES: COCCOc1nnc(-c2cc(Cl)ccc2F)cc1NCc1ccc(OC)cc1OC]

Intermediate 106 was prepared following the procedure used for the synthesis of Intermediate 8, starting from 6-chloro-N-[(2,4-dimethoxyphenyl)methyl]-3-(2-methoxyethoxy)pyridazin-4-amine (Intermediate 105, 691 mg, 1.95 mmol) and 5-chloro-2-fluorobenzeneboronic acid (511 mg, 2.93 mmol) in presence of Pd(dppf)Cl₂ (286 mg, 0.39 mmol) to afford title compound (607 mg, 1.35 mmol, 69% yield).

LC-MS (ESI): m/z (M+1): 448.3 (Method 1)

### Intermediate 107: 6-(5-chloro-2-fluorophenyl)-3-(2-methoxyethoxy)pyridazin-4-amine

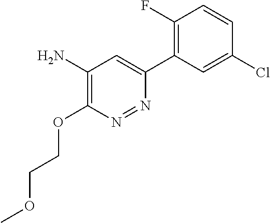 [SMILES: COCCOc1nnc(-c2cc(Cl)ccc2F)cc1N]

Intermediate 107 was prepared following the procedure used for the synthesis of Intermediate 9, starting from 6-(5-chloro-2-fluorophenyl)-N-[(2,4-dimethoxyphenyl)methyl]-3-(2-methoxyethoxy)pyridazin-4-amine (Intermediate 106, 607 mg, 1.35 mmol) to afford title compound (350 mg, 1.18 mmol, 87% yield).

LC-MS (ESI): m/z (M+1): 298.1 (Method 1)

### Intermediate 108: N-(4-bromopyridin-2-yl)-3-(morpholin-4-yl)propanamide

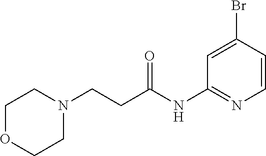 [SMILES: O=C(CCN1CCOCC1)Nc1cc(Br)ccn1]

Intermediate 108 was prepared following the procedure used for the synthesis of Intermediate 2, starting from N-(4-bromopyridin-2-yl)prop-2-enamide (Intermediate 1, 450 mg, 1.98 mmol) and morpholine (0.38 mL, 4.36 mmol) to afford title compound (540 mg, 1.72 mmol, 87% yield). LC-MS (ESI): m/z (M+1): 314.1 (Method 1)

### Intermediate 109: 6-chloro-N-[(2,4-dimethoxyphenyl)methyl]-3-[2-(4-methylpiperazin-1-yl)ethoxy]pyridazin-4-amine

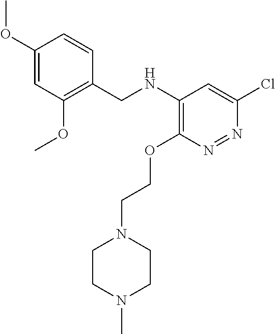 [SMILES: COc1ccc(CNc2cc(Cl)nnc2OCCN2CCN(C)CC2)c(OC)c1]

To a solution of 2-(4-methylpiperazin-1-yl)ethan-1-ol (1.38 g, 9.55 mmol) in DMF (7 mL), NaH 60% dispersion in oil (382 mg, 9.55 mmol) was added and the mixture was stirred at RT for 1.5 hrs. 3,6-dichloro-N-[(2,4-dimethoxyphenyl)methyl]pyridazin-4-amine (Intermediate 6, 1 g, 3.18 mmol) dissolved in DMF (3 mL) was added and the mixture was stirred at 130° C. overnight. The mixture was allowed to cool to room temperature, poured into saturated NaHCO₃ aqueous solution and extracted with EtOAc. The organic phase was separated, filtered through a hydrophobic phase separator, and concentrated at reduced pressure. The crude was purified by flash chromatography on Biotage silica NH cartridge (from DCM to 3% MeOH). Evaporation of opportune fractions provided title compound (608 mg, 1.44 mmol, 45% yield).

LC-MS (ESI): m/z (M+1): 422.6 (Method 1)

### Intermediate 110: 6-(5-chloro-2-fluorophenyl)-N-[(2,4-dimethoxyphenyl)methyl]-3-[2-(4-methylpiperazin-1-yl)ethoxy]pyridazin-4-amine

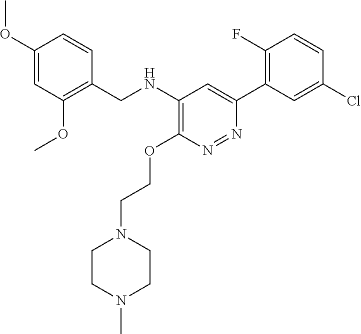 [SMILES: COc1ccc(CNc2cc(-c3cc(Cl)ccc3F)nnc2OCCN2CCN(C)CC2)c(OC)c1]

Intermediate 110 was prepared following the procedure used for the synthesis of Intermediate 8, starting from 6-chloro-N-[(2,4-dimethoxyphenyl)methyl]-3-[2-(4-methylpiperazin-1-yl)ethoxy]pyridazin-4-amine (Intermediate 109, 608 mg, 1.44 mmol) and 5-chloro-2-fluorobenzeneboronic acid (376 mg, 2.16 mmol) in presence of Pd(dppf)Cl₂ (211 mg, 0.29 mmol) to afford title compound (457 mg, 0.89 mmol, 61% yield). LC-MS (ESI): m/z (M+1): 516.3 (Method 2)

### Intermediate 111: 6-(5-chloro-2-fluorophenyl)-3-[2-(4-methylpiperazin-1-yl)ethoxy]pyridazin-4-amine

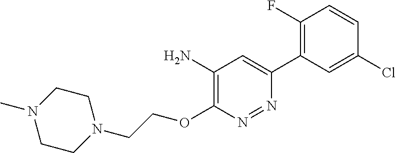 [SMILES: CN1CCN(CCOc2nnc(-c3cc(Cl)ccc3F)cc2N)CC1]

Intermediate 111 was prepared following the procedure used for the synthesis of Intermediate 9, starting from 6-(5-chloro-2-fluorophenyl)-N-[(2,4-dimethoxyphenyl)methyl]-3-[2-(4-methylpiperazin-1-yl)ethoxy]pyridazin-4-amine (Intermediate 110, 457 mg, 0.89 mmol) to afford title compound (281 mg, 0.77 mmol, 87% yield). LC-MS (ESI): m/z (M+1): 366.2 (Method 2)

### Intermediate 112: N-(4-bromopyridin-2-yl)cyclopropanecarboxamide

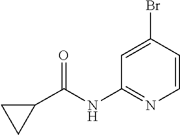 [SMILES: O=C(Nc1cc(Br)ccn1)C1CC1]

To an ice-cooled mixture of 4-bromopyridin-2-amine (500 mg, 2.89 mmol) and pyridine (0.7 mL, 8.67 mmol) in DCM (15 mL), cyclopropanecarbonyl chloride (0.31 mL, 3.47 mmol) was added dropwise and the mixture was stirred at 0° C. for 1 h. The reaction was quenched and washed with saturated NH₄Cl aqueous solution and the organic phase was separated, filtered through a hydrophobic phase separator, and concentrated at reduced pressure. The crude was purified by flash chromatography on Biotage silica cartridge (from DCM to 15% EtOAc). Evaporation of opportune fractions provided title compound (712 mg, 2.95 mmol, quantitative yield).

LC-MS (ESI): m/z (M+1): 240.9 (Method 1)

### Intermediate 113: 4-chloro-1-{[2-(trimethylsilyl)ethoxy]methyl}-1H-pyrrolo[2,3-b]pyridine

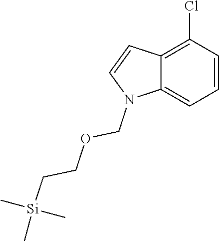 [SMILES: C[Si](C)(C)CCOCn1ccc2c(Cl)cccc21]

Intermediate 113 was prepared following the procedure used for the synthesis of Intermediate 23, starting from 4-chloro-1H-pyrrolo[2,3-b]pyridine (1 g, 6.55 mmol) and 2-(chloromethoxy)ethyl-trimethylsilane (1.5 mL, 8.52 mmol) in DMF (16 mL) to afford title compound (1.96 g, recovery assumed quantitative).

LC-MS (ESI): m/z (M+1): 283.1 (Method 1)

### Intermediate 114: 6-(5-chloro-2-fluorophenyl)-3-[2-(4-methylpiperazin-1-yl)ethoxy]-N-(1-{[2-(trimethylsilyl)ethoxy]methyl}-1H-pyrrolo[2,3-b]pyridin-4-yl)pyridazin-4-amine

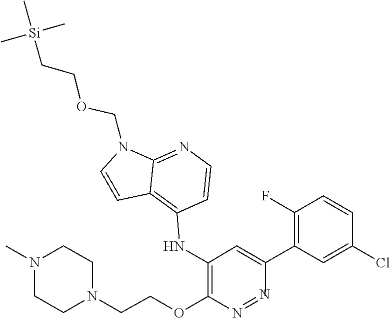

Intermediate 114 was prepared following the procedure used for the synthesis of Intermediate 47, starting from 6-(5-chloro-2-fluorophenyl)-3-[2-(4-methylpiperazin-1-yl)ethoxy]pyridazin-4-amine (Intermediate 111, 90 mg, 0.25 mmol) and 4-chloro-1-{[2-(trimethylsilyl)ethoxy]methyl}-1H-pyrrolo[2,3-b]pyridine (Intermediate 113, 83 mg, 0.3 mmol) to afford title compound (79 mg, 0.13 mmol, 52% yield).

LC-MS (ESI): m/z (M+1): 612.5 (Method 2)

### Intermediate 115: 6-(5-chloro-2-fluorophenyl)-3-[2-(dimethylamino)ethoxy]-N-(1-{[2-(trimethylsilyl)ethoxy]methyl}-1H-pyrrolo[2,3-b]pyridin-4-yl)pyridazin-4-amine

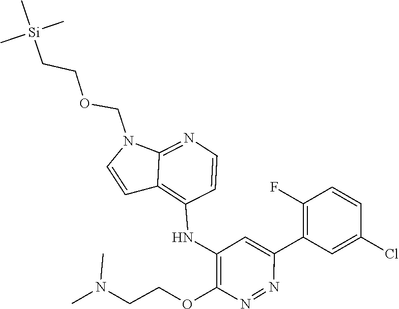 [SMILES: CN(C)CCOc1nnc(-c2cc(Cl)ccc2F)cc1Nc1ccnc2c1ccn2COCC[Si](C)(C)C]

Intermediate 115 was prepared following the procedure used for the synthesis of Intermediate 47, starting from 6-(5-chloro-2-fluorophenyl)-3-[2-(dimethylamino)ethoxy]pyridazin-4-amine (Intermediate 30, 110 mg, 0.35 mmol) and 4-chloro-1-{[2-(trimethylsilyl)ethoxy]methyl}-1H-pyrrolo[2,3-b]pyridine (Intermediate 113, 120 mg, 0.42 mmol) to afford title compound (84 mg, 0.15 mmol, 43% yield).

LC-MS (ESI): m/z (M+1): 557.3 (Method 2)

### Intermediate 116: 4-chloro-1-{[2-(trimethylsilyl)ethoxy]methyl}-1H-pyrazolo[3,4-b]pyridine

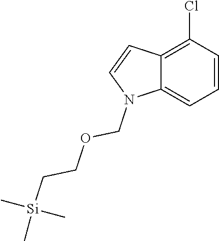 [SMILES: C[Si](C)(C)CCOCn1ccc2c(Cl)cccc21]

A mixture of 4-chloro-1H-pyrazolo[3,4-b]pyridine (500 mg, 3.26 mmol) and K₂CO₃ (1.35 g, 9.77 mmol) in DMF (16.7 mL) was stirred at RT for 30 minutes then 2-(chloromethoxy)ethyl-trimethylsilane (0.92 mL, 5.2 mmol) was added and the mixture was stirred at RT for 7 hrs. The mixture was diluted with EtOAc and washed with saturated NaHCO₃ solution (3×) and brine (1×). The organic phase was filtered through a phase separator and concentrated under vacuum. The crude was purified by flash chromatography on Biotage silica NH cartridge (from c-Hex to 15% EtOAc), then further purified by flash chromatography on Biotage silica cartridge (from c-Hex to 20% EtOAc) to afford title compound (535 mg, 1.88 mmol, 58% yield).

¹H NMR (400 MHz, DMSO-d₆) δ ppm 8.57 (d, J=5.06 Hz, 1H), 8.38 (s, 1H), 7.46 (d, J=5.06 Hz, 1H), 5.80 (s, 2H), 3.57-3.64 (m, 2H), 0.83 (d, J=8.14 Hz, 2H), −0.11 (s, 9H).

### Intermediate 117: 6-(5-chloro-2-fluorophenyl)-3-[2-(dimethylamino)ethoxy]-N-(1-{[2-(trimethylsilyl)ethoxy]methyl}-1H-pyrazolo[3,4-b]pyridin-4-yl)pyridazin-4-amine

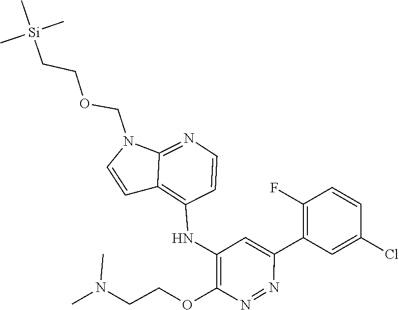 [SMILES: CN(C)CCOc1nnc(-c2cc(Cl)ccc2F)cc1Nc1ccnc2c1ccn2COCC[Si](C)(C)C]

Intermediate 117 was prepared following the procedure used for the synthesis of Intermediate 47, starting from 6-(5-chloro-2-fluorophenyl)-3-[2-(dimethylamino)ethoxy]pyridazin-4-amine (Intermediate 30, 90 mg, 0.29 mmol) and 4-chloro-1-{[2-(trimethylsilyl)ethoxy]methyl}-1H-pyrazolo[3,4-b]pyridine (Intermediate 116, 99 mg, 0.35 mmol) to afford title compound (113 mg, 0.20 mmol, 70% yield).

LC-MS (ESI): m/z (M+1): 558.4 (Method 2)

### Intermediate 118: tert-butyl N-(4-{[6-(5-chloro-2-fluorophenyl)-3-(2-hydroxyethoxy)pyridazin-4-yl]amino}pyridin-2-yl)carbamate

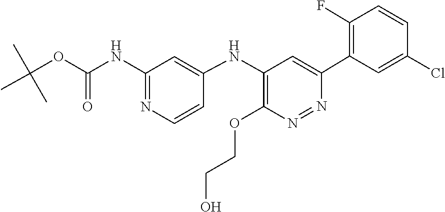 [SMILES: CC(C)(C)OC(=O)Nc1cc(Nc2cc(-c3cc(Cl)ccc3F)nnc2OCCO)ccn1]

Intermediate 118 was prepared following the procedure used for the synthesis of Intermediate 47, starting from 2-{[4-amino-6-(5-chloro-2-fluorophenyl)pyridazin-3-yl]oxy}ethan-1-ol (Intermediate 4, 193 mg, 0.63 mmol) and tert-butyl N-(4-bromopyridin-2-yl)carbamate (191 mg, 0.70 mmol) to afford title compound (100 mg, 0.21 mmol, 33% yield). LC-MS (ESI): m/z (M+1): 476.3 (Method 2)

### Intermediate 119: 6-chloro-N-[(2,4-dimethoxyphenyl)methyl]-3-[(1-methylazetidin-3-yl)methoxy]pyridazin-4-amine

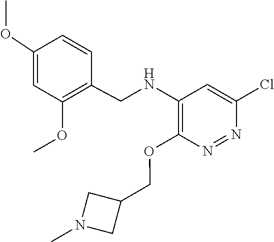 [SMILES: COc1ccc(CNc2cc(Cl)nnc2OCC2CN(C)C2)c(OC)c1]

Intermediate 119 was prepared following the procedure used for the synthesis of Intermediate 7, starting from 3,6-dichloro-N-[(2,4-dimethoxyphenyl)methyl]pyridazin-4-amine (Intermediate 6, 1.1 g, 3.5 mmol) and (1-methylazetidin-3-yl)methanol (460 mg, 4.55 mmol) at 120° C., to afford title compound (665 mg, 1.75 mmol, 50% yield).

LC-MS (ESI): m/z (M+1): 379.2 (Method 1)

### Intermediate 120: 6-(5-chloro-2-fluorophenyl)-N-[(2,4-dimethoxyphenyl)methyl]-3-[(1-methylazetidin-3-yl)methoxy]pyridazin-4-amine

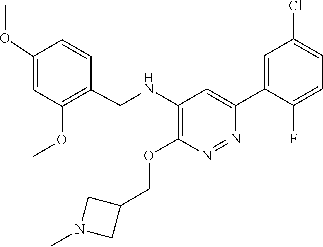 [SMILES: COc1ccc(CNc2cc(-c3cc(Cl)ccc3F)nnc2OCC2CN(C)C2)c(OC)c1]

In a suitable vial, a mixture of 6-chloro-N-[(2,4-dimethoxyphenyl)methyl]-3-[(1-methylazetidin-3-yl)methoxy]pyridazin-4-amine (Intermediate 119, 475 mg, 1.25 mmol), 5-chloro-2-fluorobenzeneboronic acid (284 mg, 1.63 mmol), Na₂CO₃ (266 mg, 2.51 mmol) and Pd(PPh₃)₄ (73 mg, 0.06 mmol) was suspended in toluene (5 mL)/ethanol (1.7 mL)/water (1.7 mL). The vial was sealed, evacuated, backfilled with N₂, and heated at 110° C. under stirring, overnight. Further 5-chloro-2-fluorobenzeneboronic acid (200 mg, 1.15 mmol) and Pd(PPh₃)₄(73 mg, 0.06 mmol) were added again and the mixture was heated for 7 hrs. The mixture was diluted with EtOAc, filtered through a Celite® pad, washing with EtOAc. The organic phase was washed with brine, separated, filtered through a phase separator, and evaporated under vacuum. The crude material was purified by flash chromatography on Biotage silica NH cartridge (from c-Hex to 100% of EtOAc), affording title compound (204 mg, 0.43 mmol, 34% yield).

LC-MS (ESI): m/z (M+1): 473.2 (Method 1)

### Intermediate 121: 6-(5-chloro-2-fluorophenyl)-3-[(1-methylazetidin-3-yl)methoxy]pyridazin-4-amine

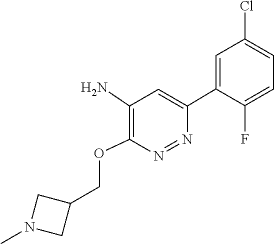

Intermediate 121 was prepared following the procedure used for the synthesis of Intermediate 9, starting from 6-(5-chloro-2-fluorophenyl)-N-[(2,4-dimethoxyphenyl)methyl]-3-[(1-methylazetidin-3-yl)methoxy]pyridazin-4-amine (Intermediate 120, 144 mg, 0.30 mmol) to afford title compound (95 mg, 0.29 mmol, 97% yield). LC-MS (ESI): m/z (M+1): 323.1 (Method 2)

### Intermediate 122: tert-butyl N-[2-({4-[(2-{[(tert-butoxy)carbonyl]amino}pyridin-4-yl)amino]-6-(5-chloro-2-fluorophenyl)pyridazin-3-yl}oxy)ethyl]-N-methanesulfonylcarbamate

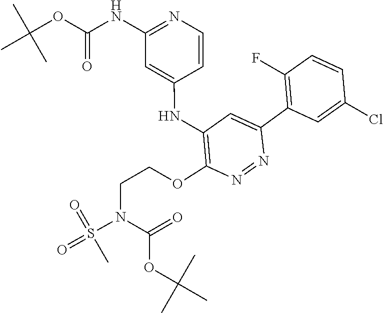 [SMILES: CC(C)(C)OC(=O)Nc1cc(Nc2cc(-c3cc(Cl)ccc3F)nnc2OCCN(C(=O)OC(C)(C)C)S(C)(=O)=O)ccn1]

To a solution of tert-butyl N-(4-{[6-(5-chloro-2-fluorophenyl)-3-(2-hydroxyethoxy)pyridazin-4-yl]amino}pyridin-2-yl)carbamate (Intermediate 118, 75 mg, 0.14 mmol) in anhydrous THE (5 mL), at RT and under N₂, tert-butyl N-methylsulfonylcarbamate (30 mg, 0.15 mmol) and PPh₃ (40 mg, 0.15 mmol) were added, followed by diisopropyl azodicarboxylate (0.03 mL, 0.15 mmol). The yellow solution was stirred for 1 hr at RT, then further tert-butyl N-methylsulfonylcarbamate (30 mg, 0.15 mmol), PPh₃ (40 mg, 0.15 mmol) and diisopropyl azodicarboxylate (0.03 mL, 0.15 mmol) were added. The mixture was heated at 55° C. for 1 h. Further tert-butyl N-methylsulfonylcarbamate (90 mg, 0.45 mmol), PPh₃ (120 mg, 0.45 mmol) and diisopropyl azodicarboxylate (0.09 mL, 0.45 mmol) were added, and after 1 h at 55° C. conversion was complete. The mixture was concentrated under reduced pressure and the residue was purified by reverse flash chromatography on Biotage C18 cartridge (from H₂O+0.1% HCOOH to 60% MeCN+0.1% HCOOH) to afford title compound (130 mg, recovery assumed quantitative). LC-MS (ESI): m/z (M+1): 653.3 (Method 1)

### Intermediate 123: 6-chloro-N-[(2,4-dimethoxyphenyl)methyl]-3-(2,2,2-trifluoroethoxy)pyridazin-4-amine

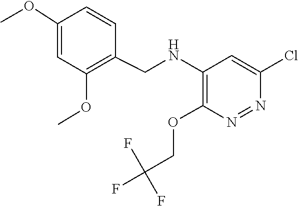

Intermediate 123 was prepared following the procedure used for the synthesis of Intermediate 7, starting from 3,6-dichloro-N-[(2,4-dimethoxyphenyl)methyl]pyridazin-4-amine (Intermediate 6, 100 mg, 0.32 mmol) and 2,2,2-trifluoroethanol (30 μL, 0.41 mmol), to afford title compound (88 mg, 0.23 mmol, 73% yield).

LC-MS (ESI): m/z (M+1): 378.2 (Method 1)

### Intermediate 124: 6-(5-chloro-2-fluorophenyl)-N-[(2,4-dimethoxyphenyl)methyl]-3-(2,2,2-trifluoroethoxy)pyridazin-4-amine

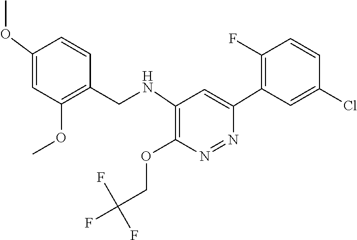

Intermediate 124 was prepared following the procedure used for the synthesis of Intermediate 8, starting from 6-chloro-N-[(2,4-dimethoxyphenyl)methyl]-3-(2,2,2-trifluoroethoxy)pyridazin-4-amine (Intermediate 123, 88 mg, 0.23 mmol) and 5-chloro-2-fluorobenzeneboronic acid (61 mg, 0.31 mmol) in presence of Pd(dppf)Cl₂ (34 mg, 0.05 mmol) to afford title compound (88 mg, 0.19 mmol, 80% yield).

LC-MS (ESI): m/z (M+1): 472.2 (Method 1)

### Intermediate 125: 6-(5-chloro-2-fluorophenyl)-3-(2,2,2-trifluoroethoxy)pyridazin-4-amine

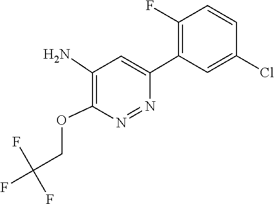

Intermediate 125 was prepared following the procedure used for the synthesis of Intermediate 9, starting from 6-(5-chloro-2-fluorophenyl)-N-[(2,4-dimethoxyphenyl)methyl]-3-(2,2,2-trifluoroethoxy)pyridazin-4-amine (Intermediate 124, 88 mg, 0.19 mmol) to afford title compound (48 mg, 0.15 mmol, 80% yield).

LC-MS (ESI): m/z (M+1): 322.1 (Method 1)

### Intermediate 126: tert-butyl N-(4-{[6-(5-chloro-2-fluorophenyl)-3-(2,2,2-trifluoroethoxy)pyridazin-4-yl]amino}pyridin-2-yl)carbamate

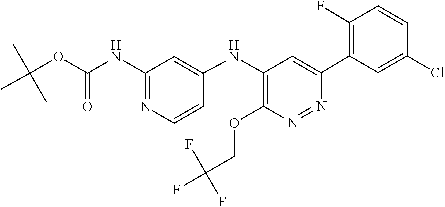 [SMILES: CC(C)(C)OC(=O)Nc1cc(Nc2cc(-c3cc(Cl)ccc3F)nnc2OCC(F)(F)F)ccn1]

Intermediate 126 was prepared following the procedure used for the synthesis of Intermediate 47, starting from 6-(5-chloro-2-fluorophenyl)-3-(2,2,2-trifluoroethoxy)pyridazin-4-amine (Intermediate 125, 48 mg, 0.15 mmol) and tert-butyl N-(4-bromopyridin-2-yl)carbamate (45 mg, 0.16 mmol) to afford title compound (50 mg, 0.10 mmol, 65% yield). LC-MS (ESI): m/z (M+1): 514.2 (Method 1)

### Intermediate 127: tert-butyl N-(4-{[6-(5-chloro-2-fluorophenyl)-3-(2,2-difluoroethoxy)pyridazin-4-yl]amino}pyridin-2-yl)carbamate

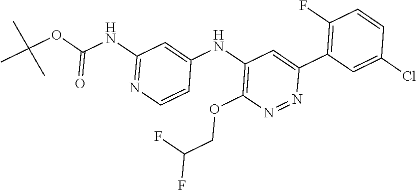

Intermediate 127 was prepared following the procedure used for the synthesis of Intermediate 47, starting from 6-(5-chloro-2-fluorophenyl)-3-(2,2-difluoroethoxy)pyridazin-4-amine (Intermediate 9, 60 mg, 0.20 mmol) and tert-butyl N-(4-bromopyridin-2-yl)carbamate (59 mg, 0.22 mmol) to afford title compound (60 mg, 0.12 mmol, 61% yield). LC-MS (ESI): m/z (M+1): 496.2 (Method 1)

### Intermediate 128: 6-chloro-N-[(2,4-dimethoxyphenyl)methyl]-3-[2-(pyrrolidin-1-yl)ethoxy]pyridazin-4-amine

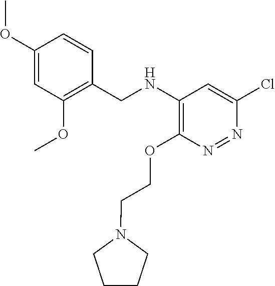 [SMILES: COc1ccc(CNc2cc(Cl)nnc2OCCN2CCCC2)c(OC)c1]

Intermediate 128 was prepared following the procedure used for the synthesis of Intermediate 109, starting from 3,6-dichloro-N-[(2,4-dimethoxyphenyl)methyl]pyridazin-4-amine (Intermediate 6, 500 mg, 1.06 mmol) and 2-pyrrolidin-1-ylethanol (550 mg, 4.77 mmol) to afford title compound (664 mg, recovery assumed quantitative). LC-MS (ESI): m/z (M+1): 393.2 (Method 2)

### Intermediate 129: 6-(5-chloro-2-fluorophenyl)-N-[(2,4-dimethoxyphenyl)methyl]-3-[2-(pyrrolidin-1-yl)ethoxy]pyridazin-4-amine

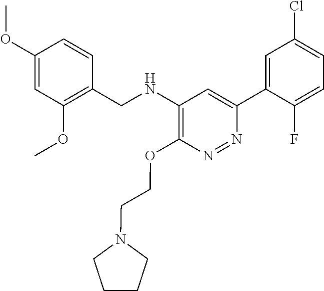 [SMILES: COc1ccc(CNc2cc(-c3cc(Cl)ccc3F)nnc2OCCN2CCCC2)c(OC)c1]

Intermediate 129 was prepared following the procedure used for the synthesis of Intermediate 8, starting from 6-chloro-N-[(2,4-dimethoxyphenyl)methyl]-3-[2-(pyrrolidin-1-yl)ethoxy]pyridazin-4-amine (Intermediate 128, 664 mg, 1.06 mmol) and 5-chloro-2-fluorobenzeneboronic acid (287 mg, 1.65 mmol) in presence of Pd(dppf)Cl₂ (161 mg, 0.22 mmol) to afford title compound (355 mg, 0.73 mmol, 66% yield).

LC-MS (ESI): m/z (M+1): 487.4 (Method 2)

### Intermediate 130: 6-(5-chloro-2-fluorophenyl)-3-[2-(pyrrolidin-1-yl)ethoxy]pyridazin-4-amine

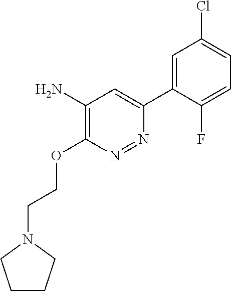 [SMILES: Nc1cc(-c2cc(Cl)ccc2F)nnc1OCCN1CCCC1]

Intermediate 130 was prepared following the procedure used for the synthesis of Intermediate 9, starting from 6-(5-chloro-2-fluorophenyl)-N-[(2,4-dimethoxyphenyl)methyl]-3-[2-(pyrrolidin-1-yl)ethoxy]pyridazin-4-amine (Intermediate 129, 355 mg, 0.73 mmol) to afford title compound (233 mg, 0.69 mmol, 95% yield).

LC-MS (ESI): m/z (M+1): 337.1 (Method 2)

### Intermediate 131: tert-butyl N-(4-{[6-(5-chloro-2-fluorophenyl)-3-[3-(methylsulfanyl)propoxy]pyridazin-4-yl]amino}pyridin-2-yl)carbamate

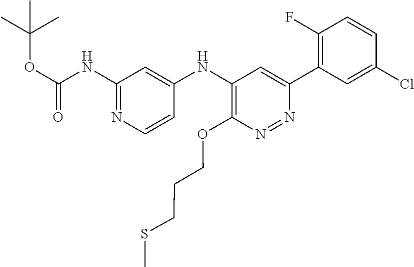

Intermediate 131 was prepared following the procedure used for the synthesis of Intermediate 47, starting from 6-(5-chloro-2-fluorophenyl)-3-[3-(methylsulfanyl)propoxy]pyridazin-4-amine (Intermediate 12, 50 mg, 0.15 mmol) and tert-butyl N-(4-bromopyridin-2-yl)carbamate (46 mg, 0.17 mmol) to afford title compound (50 mg, 0.10 mmol, 63% yield). LC-MS (ESI): m/z (M+1): 520.2 (Method 1)

### Intermediates 132 and 133: tert-butyl N-(4-{[6-(5-chloro-2-fluorophenyl)-3-(3-methanesulfonylpropoxy)pyridazin-4-yl]amino}pyridin-2-yl)carbamate (Int 132) and tert-butyl N-(4-{[6-(5-chloro-2-fluorophenyl)-3-(3-methanesulfinylpropoxy) pyridazin-4-yl]amino}pyridin-2-yl)carbamate (Int 133)

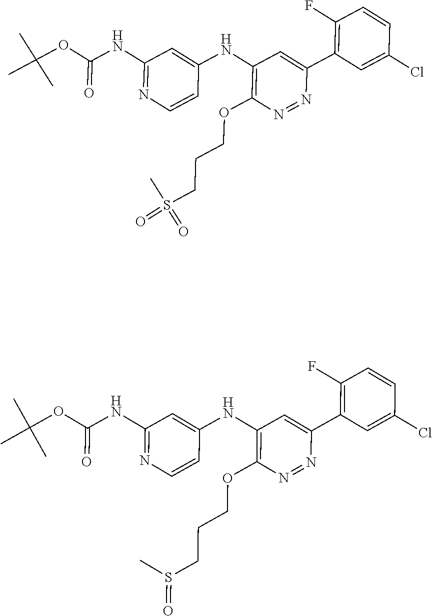

tert-butyl N-(4-{[6-(5-chloro-2-fluorophenyl)-3-[3-(methylsulfanyl)propoxy]pyridazin-4-yl]amino}pyridin-2-yl)carbamate (Intermediate 131, 160 mg, 0.30 mmol) was suspended in MeOH (6 mL) and a solution of Oxone® (136 mg, 0.44 mmol) in H₂O (2 mL) was added. The resulting suspension was stirred at RT for 55 min. Saturated NaHCO₃ solution was added to adjust the pH to 8, then EtOAc was added, and products were extracted with EtOAc 3 x. Organic phases were collected, evaporated, and dried, the residue was purified by flash chromatography on Biotage silica cartridge (from 50% c-Hex to 100% EtOAc, then to 30% MeOH in EtOAc). Opportune fractions were collected to afford tert-butyl N-(4-{[6-(5-chloro-2-fluorophenyl)-3-(3-methanesulfonylpropoxy)pyridazin-4-yl]amino}pyridin-2-yl)carbamate (Int 132, 70 mg, 0.13 mmol, 43% yield) and tert-butyl N-(4-{[6-(5-chloro-2-fluorophenyl)-3-(3-methanesulfinylpropoxy)pyridazin-4-yl]amino}pyridin-2-yl)carbamate (Int 133, 75 mg, 0.14 mmol, 47% yield).

Int 132: LC-MS (ESI): m/z (M+1): 552.2 (Method 1)

Int 133: LC-MS (ESI): m/z (M+1): 536.3 (Method 1)

### Intermediate 134: N-(4-{[6-(5-chloro-2-fluorophenyl)-3-[3-(dimethylamino)propoxy]pyridazin-4-yl]amino}pyridin-2-yl)cyclopropanecarboxamide

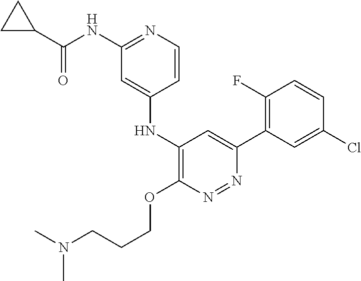

Intermediate 134 was prepared following the procedure used for the synthesis of Intermediate 47, starting from 6-(5-chloro-2-fluorophenyl)-3-[3-(dimethylamino)propoxy]pyridazin-4-amine (Intermediate 26, 110 mg, 0.33 mmol) and N-(4-bromopyridin-2-yl)cyclopropanecarboxamide (Intermediate 112, 91 mg, 0.36 mmol) to afford title compound (72 mg, 0.15 mmol, 45% yield).

LC-MS (ESI): m/z (M+1): 485.2 (Method 1)

### Intermediate 135: 6-chloro-3-(oxolan-3-yloxy)pyridazin-4-amine

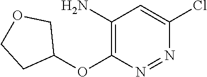 [SMILES: Nc1cc(Cl)nnc1OC1CCOC1]

To a solution of oxolan-3-ol (1.48 mL, 18.29 mmol) in DMF (33.3 mL), NaH 60% dispersion in oil (731 mg, 18.29 mmol) was added and the mixture was stirred at RT for 1.5 h (until gas evolution ceased). 3,6-dichloropyridazin-4-amine (1 g, 6.1 mmol) dissolved in DMF (13.3 mL) was added and the reaction warmed ad 130° C. for 3 hrs. The mixture was diluted with EtOAc and washed with saturated NaHCO₃ solution (3×). Aqueous phase was further extracted with EtOAc (3×) and the combined organic layers were filtered through a phase separator and concentrated under vacuum. To remove the residual DMF, n-heptane was added, and the solvents were evaporated under vacuum. This was repeated 3 times. Since DMF was still present, the mixture was loaded on SCX (20 g), washing with MeOH and then 1 N NH₃ in MeOH. Basic fractions were evaporated and then triturated with DCM to afford a first batch of title compound (100 mg, 0.46 mmol). The methanolic fraction was evaporated to give a crude containing the formyl derivative (3.88 g). This material was dissolved with ethanol (12.5 mL), 2 N NaOH (2.5 mL, 5 mmol) was added, and the mixture was heated at 65° C. for 30 min. The ethanol was concentrated under vacuum. The residue was diluted with water and extracted with EtOAc (3×). The combined organic layers were filtered through a phase separator and concentrated under vacuum. The crude material was purified by flash chromatography on Biotage silica NH cartridge (from c-Hex to 50% of EtOAc), opportune fractions were collected, mixed with the former batch, and evaporated to afford title compound (655 mg, 3.03 mmol, 50% yield). LC-MS (ESI): m/z (M+1): 216.0 (Method 1)

### Intermediate 136: 6-(5-chloro-2-fluorophenyl)-3-(oxolan-3-yloxy)pyridazin-4-amine

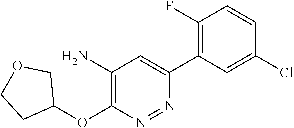 [SMILES: Nc1cc(-c2cc(Cl)ccc2F)nnc1OC1CCOC1]

Intermediate 136 was prepared following the procedure used for the synthesis of Intermediate 8, starting from 6-chloro-3-(oxolan-3-yloxy)pyridazin-4-amine (Intermediate 135, 650 mg, 3.01 mmol) and 5-chloro-2-fluorobenzeneboronic acid (788 mg, 4.52 mmol) in presence of Pd(dppf)Cl₂ (441 mg, 0.60 mmol) to afford title compound (562 mg, 1.82 mmol, 61% yield). LC-MS (ESI): m/z (M+1): 310.1 (Method 2)

### Intermediate 137: tert-butyl 4-{[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]methyl}piperazine-1-carboxylate

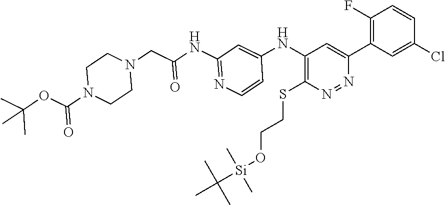

Intermediate 137 was prepared following the procedure used for the synthesis of Intermediate 47, starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and tert-butyl 4-{[(4-bromopyridin-2-yl)carbamoyl]methyl}piperazine-1-carboxylate (Intermediate 39, 110 mg, 0.27 mmol) to afford title compound (130 mg, 0.18 mmol, 73% yield). LC-MS (ESI): m/z (M+1): 732.4 (Method 2)

### Intermediate 138: tert-butyl 4-{[(4-bromopyridin-2-yl)carbamoyl]methyl}-1,4-diazepane-1-carboxylate

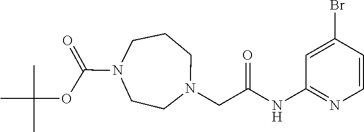 [SMILES: CC(C)(C)OC(=O)N1CCCN(CC(=O)Nc2cc(Br)ccn2)CC1]

Intermediate 138 was prepared following the procedure used for the synthesis of Intermediate 39, starting from N-(4-bromopyridin-2-yl)-2-chloroacetamide (Intermediate 33, 600 mg, 2.40 mmol) and tert-butyl 1,4-diazepane-1-carboxylate (722 mg, 3.60 mmol) to afford title compound (740 mg, 1.79 mmol, 74% yield).

LC-MS (ESI): m/z (M+1): 414.3 (Method 2)

### Intermediate 139: tert-butyl 4-{[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]methyl}-1,4-diazepane-1-carboxylate

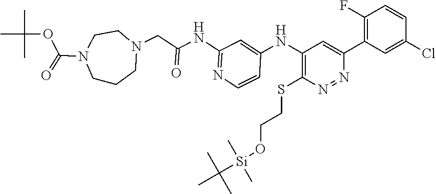 [SMILES: CC(C)(C)OC(=O)N1CCCN(CC(=O)Nc2cc(Nc3cc(-c4cc(Cl)ccc4F)nnc3SCCO[Si](C)(C)C(C)(C)C)ccn2)CC1]

Intermediate 139 was prepared following the procedure used for the synthesis of Intermediate 47, starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and tert-butyl 4-{[(4-bromopyridin-2-yl)carbamoyl]methyl}-1,4-diazepane-1-carboxylate (Intermediate 138, 110 mg, 0.27 mmol) to afford title compound (106 mg, 0.14 mmol, 59% yield). LC-MS (ESI): m/z (M+1): 746.4 (Method 2)

### Intermediate 140: tert-butyl 4-{2-[(4-{[6-(5-chloro-2-fluorophenyl)-3-(methylsulfanyl)pyridazin-4-yl]amino}pyridin-2-yl)carbamoyl]ethyl}piperazine-1-carboxylate

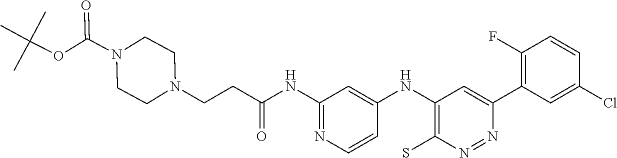

Intermediate 140 was prepared following the procedure used for the synthesis of Intermediate 47, starting from 6-(5-chloro-2-fluorophenyl)-3-(methylsulfanyl)pyridazin-4-amine (Intermediate 50, 100 mg, 0.37 mmol) and tert-butyl 4-{2-[(4-bromopyridin-2-yl)carbamoyl]ethyl}piperazine-1-carboxylate (Intermediate 57, 169 mg, 0.41 mmol) to afford title compound (110 mg, 0.18 mmol, 49% yield).

LC-MS (ESI): m/z (M+1): 602.3 (Method 2)

### Intermediate 141: N-(4-bromopyridin-2-yl)-2-[(1R,4R)-5-methyl-2,5-diazabicyclo[2.2.1]heptan-2-yl]acetamide

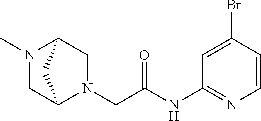 [SMILES: CN1C[C@H]2C[C@@H]1CN2CC(=O)Nc1cc(Br)ccn1]

Intermediate 141 was prepared following the procedure used for the synthesis of Intermediate 72, starting from N-(4-bromopyridin-2-yl)-2-chloroacetamide (Intermediate 33, 300 mg, 1.20 mmol) and (1R,4R)-2-methyl-2,5-diazabicyclo[2.2.1]heptane dihydrochloride (289 mg, 1.53 mmol) to afford title compound (282 mg, 0.87 mmol, 72% yield). LC-MS (ESI): m/z (M+1): 325.1 (Method 2)

### Intermediate 142: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-[(1R,4R)-5-methyl-2,5-diazabicyclo[2.2.1]heptan-2-yl]acetamide

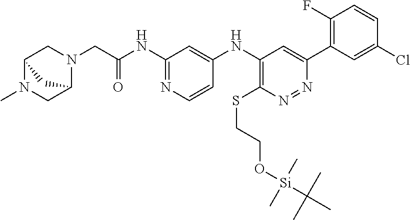 [SMILES: CN1C[C@H]2C[C@@H]1CN2CC(=O)Nc1cc(Nc2cc(-c3cc(Cl)ccc3F)nnc2SCCO[Si](C)(C)C(C)(C)C)ccn1]

Intermediate 142 was prepared following the procedure used for the synthesis of Intermediate 47, starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and N-(4-bromopyridin-2-yl)-2-[(1R,4R)-5-methyl-2,5-diazabicyclo[2.2.1]heptan-2-yl]acetamide (Intermediate 141, 86 mg, 0.27 mmol) to afford title compound (55 mg, 0.08 mmol, 35% yield). LC-MS (ESI): m/z (M+1): 658.4 (Method 2)

### Intermediate 143: N-(4-bromopyridin-2-yl)-2-[(1S,4S)-5-methyl-2,5-diazabicyclo[2.2.1]heptan-2-yl]acetamide

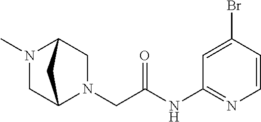 [SMILES: CN1C[C@@H]2C[C@H]1CN2CC(=O)Nc1cc(Br)ccn1]

Intermediate 143 was prepared following the procedure used for the synthesis of Intermediate 72, starting from N-(4-bromopyridin-2-yl)-2-chloroacetamide (Intermediate 33, 300 mg, 1.20 mmol) and (1S,4S)-2-methyl-2,5-diazabicyclo[2.2.1]heptane hydrobromide (424 mg, 1.55 mmol) to afford title compound (280 mg, 0.86 mmol, 72% yield). LC-MS (ESI): m/z (M+1): 325.1 (Method 2)

### Intermediate 144: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-[(1S,4S)-5-methyl-2,5-diazabicyclo[2.2.1]heptan-2-yl]acetamide

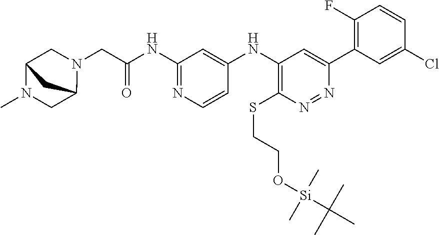 [SMILES: CN1C[C@@H]2C[C@H]1CN2CC(=O)Nc1cc(Nc2cc(-c3cc(Cl)ccc3F)nnc2SCCO[Si](C)(C)C(C)(C)C)ccn1]

Intermediate 144 was prepared following the procedure used for the synthesis of Intermediate 47, starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and N-(4-bromopyridin-2-yl)-2-[(1S,4S)-5-methyl-2,5-diazabicyclo[2.2.1]heptan-2-yl]acetamide (Intermediate 143, 86 mg, 0.27 mmol) to afford title compound (91 mg, 0.14 mmol, 57% yield). LC-MS (ESI): m/z (M+1): 658.4 (Method 2)

### Intermediate 145: 2-{[4-amino-6-(5-chloro-2-fluorophenyl)pyridazin-3-yl](methyl)amino}ethan-1-ol

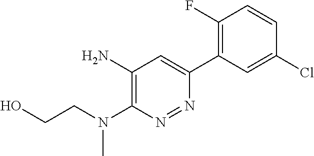 [SMILES: CN(CCO)c1nnc(-c2cc(Cl)ccc2F)cc1N]

### Step 1

2-(methylamino)ethanol (0.16 mL, 2.01 mmol), K₂CO₃ (557 mg, 4.03 mmol), and 3,6-dichloropyridazin-4-amine (330 mg, 2.01 mmol) were mixed in DMF (3 mL) and heated at 110° C. for 2 days. The mixture was charged on SCX, washed with MeOH and eluted with 1 N NH₃ in MeOH. Evaporation of basic fractions afforded a crude material that contain 24% a/a of 2-[(4-amino-6-chloro-pyridazin-3-yl)-methyl-amino]ethanol, that was used as such in the next step.

### Step 2

In a suitable vial, a mixture of 2-[(4-amino-6-chloro-pyridazin-3-yl)-methyl-amino]ethanol (2 mmol), 5-chloro-2-fluorobenzeneboronic acid (697 mg, 4 mmol), K₂CO₃ (829 mg, 6 mmol) and in 1,2-dimethoxyethane (9.6 mL) and H₂O (2.39 mL) was degassed (vacuum/N₂), then Pd(dppf)Cl₂ (293 mg, 0.40 mmol) was added. The vial was closed, and heated at 110° C. for 1 h. Further Pd(dppf)Cl₂ (293 mg, 0.40 mmol), K₂CO₃ (829 mg, 6 mmol) and 5-chloro-2-fluorobenzeneboronic acid (697 mg, 4 mmol) were added, then heated at 110° C. for 1 h. The mixture was filtered through a Celite® pad washing with EtOAc; the filtrate was concentrated at reduced pressure. The crude material was purified by reverse flash chromatography on Biotage C18 cartridge (from H₂O+0.1% NH₄OH to 40% MeCN) to afford title compound (30 mg, 0.10 mmol, 5% yield). LC-MS (ESI): m/z (M+1): 297.1 (Method 2)

### Intermediate 146: 6-(5-chloro-2-fluorophenyl)-3-(oxetan-3-yloxy)pyridazin-4-amine

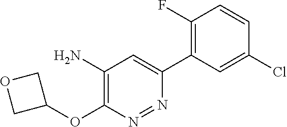 [SMILES: Nc1cc(-c2cc(Cl)ccc2F)nnc1OC1COC1]

### Step 1

3-oxetanol (0.04 mL, 0.61 mmol) and t-BuOK (75 mg, 0.67 mmol) were mixed in THE (3 mL) and stirred 10 min before adding 3,6-dichloropyridazin-4-amine (100 mg, 0.61 mmol). The resulting yellow mixture was stirred 1 h at RT, then it was heated at 70° C. overnight. The mixture was cooled to RT and charged on SCX, washing with MeOH, and eluting with 1 N NH₃ in MeOH. Evaporation of basic fractions afforded a mixture containing 44% a/a of the title compound (109 mg) that was used as such.

### Step 2

In a suitable vial, a mixture of 6-chloro-3-(oxetan-3-yloxy)pyridazin-4-amine (109 mg), 5-chloro-2-fluorobenzeneboronic acid (82 mg, 0.47 mmol), K₂CO₃ (98 mg, 0.71 mmol) and Pd(dppf)Cl₂ (34 mg, 0.05 mmol) in 1,2-dimethoxyethane (1.12 mL) and H₂O (0.28 mL) was degassed (vacuum/N₂) then heated at 110° C. for 1 h. Further Pd(dppf)Cl₂ (34 mg, 0.05 mmol), K₂CO₃ (98 mg, 0.71 mmol) and 5-chloro-2-fluorobenzeneboronic acid (82 mg, 0.47 mmol) were added, then stirred at 110° C. for 1 h. The mixture was filtered through a Celite® pad washing with EtOAc; the filtrate was concentrated at reduced pressure. The crude material was purified by reverse flash chromatography on Biotage C18 cartridge (from H₂O+0.1% NH₄OH to 40% MeCN) to afford title compound (35 mg, 0.12 mmol, 19% yield). LC-MS (ESI): m/z (M+1): 296.1 (Method 2)

### Intermediate 147: 6-chloro-3-(2,2,2-trifluoroethoxy)pyridazin-4-amine

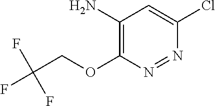 [SMILES: Nc1cc(Cl)nnc1OCC(F)(F)F]

Intermediate 147 was prepared following the procedure used for the synthesis of Intermediate 9, starting from 6-chloro-N-[(2,4-dimethoxyphenyl)methyl]-3-(2,2,2-trifluoroethoxy)pyridazin-4-amine (Intermediate 123, 454 mg, 1.20 mmol) to afford title compound (236 mg, 1.03 mmol, 86% yield). LC-MS (ESI): m/z (M+1): 228.4 (Method 2)

### Intermediate 148: N-(4-{[6-chloro-3-(2,2,2-trifluoroethoxy)pyridazin-4-yl]amino}pyridin-2-yl)-3-(4-methylpiperazin-1-yl)propanamide

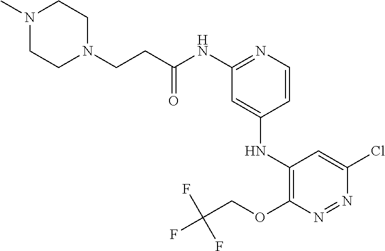 [SMILES: CN1CCN(CCC(=O)Nc2cc(Nc3cc(Cl)nnc3OCC(F)(F)F)ccn2)CC1]

Intermediate 148 was prepared following the procedure used for the synthesis of Intermediate 47, starting from 6-chloro-3-(2,2,2-trifluoroethoxy)pyridazin-4-amine (Intermediate 147, 236 mg, 1.03 mmol) and N-(4-bromopyridin-2-yl)-3-(4-methylpiperazin-1-yl)propanamide (Intermediate 2, 400 mg, 1.23 mmol) to afford title compound (293 mg, 0.62 mmol, 60% yield). LC-MS (ESI): m/z (M+1): 474.4 (Method 2)

### Intermediate 149: 6-Chloro-3-(2-methoxyethoxy)pyridazin-4-amine

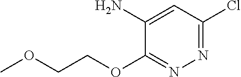

Intermediate 149 was prepared following the procedure used for the synthesis of Intermediate 3, starting from 2-methoxyethan-1-ol (1.392 g, 18.29 mmol). Purification by reverse flash chromatography on Biotage C18 cartridge (from H₂O/MeCN 95:5+0.1% HCOOH to 30% of MeCN/H₂O 95:5+0.1% HCCOH) afforded the title compound (0.5 g, 2.455 mmol, 40% yield). LC-MS (ESI): m/z (M+1): 204.2 (Method 2)

### Intermediate 150: Tert-butyl (4-((6-chloro-3-(2-methoxyethoxy)pyridazin-4-yl)amino)pyridin-2-yl)carbamate

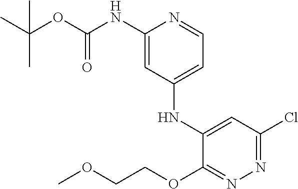 [SMILES: COCCOc1nnc(Cl)cc1Nc1ccnc(NC(=O)OC(C)(C)C)c1]

Intermediate 150 was prepared following the procedure used for the synthesis of Intermediate 18, starting from Intermediate 149 (470 mg, 2.308 mmol) and using tert-butyl (4-bromopyridin-2-yl)carbamate (630 mg, 2.308 mmol). Purification by reverse flash chromatography on Biotage C18 cartridge (from H₂O/MeCN 95:5+0.1% HCOOH to 30% of MeCN/H₂O 95:5+0.1% HCCOH) afforded the title compound (30 mg, 0.076 mmol, 3.3% yield). LC-MS (ESI): m/z (M+1): 396.3 (Method 2)

### Intermediate 151: 6-Chloro-3-(2-(4-methylpiperazin-1-yl)ethoxy)pyridazin-4-amine

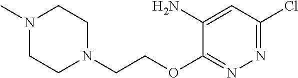 [SMILES: CN1CCN(CCOc2nnc(Cl)cc2N)CC1]

Intermediate 151 was prepared following the procedure used for the synthesis of Intermediate 3, starting from 2-(4-methylpiperazin-1-yl)ethan-1-ol (5.28 g, 36.6 mmol). The reaction was heated to 130° C. and stirred for 18 h. The reaction was cooled and DMF was removed under reduced pressure. The residue was dissolved in EtOAc (100 mL) and extracted with aquoeus 1M HCl. The aqueous layer was collected and basified with saturated aqueous K₂CO₃ solution. The resulting solution was evaporated to dryness. The solid was suspended in EtOH (40 mL), boiled for 30 min and filtered. The mother liquors were concentrated to dryness under reduced pressure and the residue was purified by flash chromatography on Biotage silica NH cartridge (from 0 to 5% of EtOH in DCM) affording the title compound (1.5 g, 5.52 mmol, 45% yield).

LC-MS (ESI): m/z (M+1): 272.3 (Method 2)

### Intermediate 152: N⁴-(6-chloro-3-(2-(4-methylpiperazin-1-yl)ethoxy)pyridazin-4-yl)pyridine-2,4-diamine

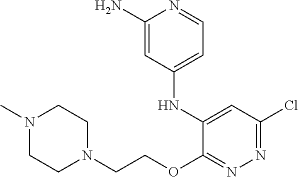 [SMILES: CN1CCN(CCOc2nnc(Cl)cc2Nc2ccnc(N)c2)CC1]

Intermediate 152 was prepared following the procedure for the synthesis of Intermediate 18, starting from Intermediate 151 (200 mg, 0.736 mmol) and using tert-butyl (4-bromopyridin-2-yl)carbamate (302 mg, 1.104 mmol). The reaction mixture was heated to 110° C. and stirred for 3 h. Then, MTBE (20 mL) was added and the organic phase was quenched with aqueous 1M HCl (10 mL). The two phases were separated and the aqueous layer was neutralized by addition of solid NaOH. Water was evaporated to dryness under reduced pressure affording the title compound, which was used as such in the next step. LC-MS (ESI): m/z (M+1): 364.4 (Method 2)

### Intermediate 151: 6-Chloro-3-(2-methoxyethoxy)pyridazin-4-amine

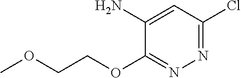

Intermediate 151 was prepared following the procedure used for the synthesis of Intermediate 3, starting from 2-methoxyethan-1-ol (1.392 g, 18.29 mmol). Purification by reverse flash chromatography on Biotage C18 cartridge (from 100% H₂O/MeCN 95:5 +0.1% HCOOH to 30% of MeCN/H₂O 95:5+0.1% HCCOH) afforded the title compound (0.5 g, 2.455 mmol, 40% yield). LC-MS (ESI): m/z (M+1): 204.2 (Method 2)

### Intermediate 152: Tert-butyl (4-((6-chloro-3-(2-methoxyethoxy)pyridazin-4-yl)amino)pyridin-2-yl)carbamate

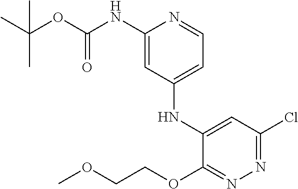 [SMILES: COCCOc1nnc(Cl)cc1Nc1ccnc(NC(=O)OC(C)(C)C)c1]

Intermediate 152 was prepared following the procedure used for the synthesis of Intermediate 18, starting from Intermediate 151 (470 mg, 2.308 mmol) and using tert-butyl (4-bromopyridin-2-yl)carbamate (630 mg, 2.308 mmol). Purification by reverse flash chromatography on Biotage C18 cartridge (from 100% H₂O/MeCN 95:5+0.1% HCOOH to 30% of MeCN/H₂O 95:5+0.1% HCCOH) afforded the title compound (30 mg, 0.076 mmol, 3.3% yield). LC-MS (ESI): m/z (M+1): 396.3 (Method 2)

### Intermediate 153: methyl 3-[(tert-butyldimethylsilyl)oxy]cyclobutane-1-carboxylate

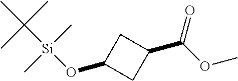

Intermediate 153 was prepared following the procedure used for the synthesis of Intermediate 65 starting from 3 methyl 3-hydroxycyclobutane-1-carboxylate (0.5 g, 3.84 mmol) to afford title compound (0.85 g, 3.53 mmol, 92% yield).

¹H NMR (400 MHz, Chloroform-d) δ ppm 4.14 (tt, J=8.2, 6.7 Hz, 1H), 3.67 (s, 3H), 2.59-2.41 (m, 3H), 2.23-2.15 (m, 2H), 0.88 (s, 9H), 0.04 (s, 6H).

### Intermediate 154: {3-[(tert-butyldimethylsilyl)oxy]cyclobutyl}methanol

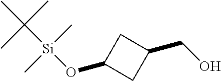

In a flame dried 2-neck flask, a solution of methyl 3-[(tert-butyldimethylsilyl)oxy]cyclobutane-1-carboxylate (Intermediate 153, 850 mg, 3.53 mmol) in THE (10 mL) was treated with 2 M lithium aluminum hydride in THE (5.3 mL, 10.61 mmol) at 0° C. under N₂ atmosphere. The mixture was stirred for 30 min at the same temperature, then 5 g of Na₂SO₄ was added followed by 20 mL of EtOAc at 0° C. The mixture was stirred for 5 minutes, then water was added till the mixture turned clear. The mixture was filtered washing with EtOAC, and the solvent removed under reduced pressure. The crude product was purified by flash chromatography on Biotage silica cartridge (from cHex to 50% EtOAc) to afford title compound (536 mg, 2.48 mmol, 70% yield).

¹H NMR (400 MHz, Chloroform-d) δ ppm 4.15 (quin, J=7.3 Hz, 1H), 3.60 (t, J=5.9 Hz, 2H), 2.34 (dtt, J=9.4, 7.0, 2.6 Hz, 2H), 2.01-1.87 (m, 1H), 1.62-1.73 (m, 2H), 1.33 (t, J=5.7 Hz, 1H), 0.88 (s, 9H), 0.04 (s, 6H)

### Intermediate 155: 3-({3-[(tert-butyldimethylsilyl)oxy]cyclobutyl}methoxy)-6-chloro-N-[(2,4-dimethoxyphenyl)methyl]pyridazin-4-amine

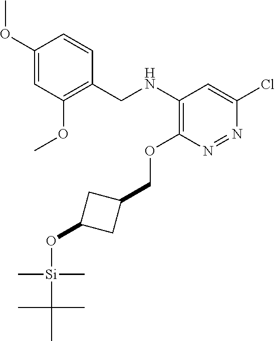

Intermediate 155 was prepared following the procedure used for the synthesis of Intermediate 7 starting from 3,6-dichloro-N-[(2,4-dimethoxyphenyl)methyl]pyridazin-4-amine (Intermediate 6, 400 mg, 1.27 mmol), and {3-[(tert-butyldimethylsilyl)oxy]cyclobutyl}methanol (Intermediate 154, 0.49 mL, 4.14 mmol) at 110° C. to afford title compound (478 mg, 0.97 mmol, 76% yield).

LC-MS (ESI): m/z (M+1): 494.3 (Method 1)

### Intermediate 156: 3-({3-[(tert-butyldimethylsilyl)oxy]cyclobutyl}methoxy)-6-(5-chloro-2-fluorophenyl)-N-[(2,4-dimethoxyphenyl)methyl]pyridazin-4-amine

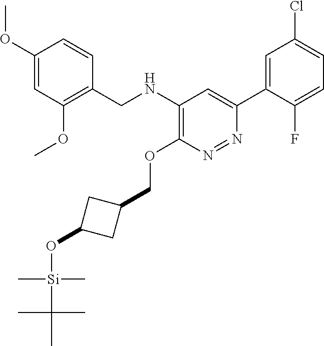

Intermediate 156 was prepared following the procedure used for the synthesis of Intermediate 8, starting from 3-({3-[(tert-butyldimethylsilyl)oxy]cyclobutyl}methoxy)-6-chloro-N-[(2,4-dimethoxyphenyl)methyl]pyridazin-4-amine (Intermediate 155, 478 mg, 0.97 mmol) and 5-chloro-2-fluorobenzeneboronic acid (253 mg, 1.45 mmol) in presence of Pd(dppf)Cl₂ (141 mg, 0.19 mmol) to afford title compound (288 mg, 0.49 mmol, 51% yield). LC-MS (ESI): m/z (M+1): 588.4 (Method 1)

### Intermediate 157: 3-({[4-amino-6-(5-chloro-2-fluorophenyl)pyridazin-3-yl]oxy}methyl)cyclobutan-1-ol

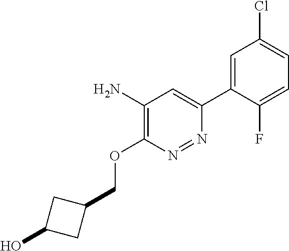

Intermediate 157 was prepared following the procedure used for the synthesis of Intermediate 64, starting from 3-({3-[(tert-butyldimethylsilyl)oxy]cyclobutyl}methoxy)-6-(5-chloro-2-fluorophenyl)-N-[(2,4-dimethoxyphenyl)methyl]pyridazin-4-amine (Intermediate 156, 288 mg, 0.49 mmol) to afford title compound (66 mg, 0.20 mmol, 42% yield). LC-MS (ESI): m/z (M+1): 324.1 (Method 1)

### Intermediate 158: 6-chloro-3-[(2,2-dimethyl-1,3-dioxolan-4-yl)methoxy]pyridazin-4-amine

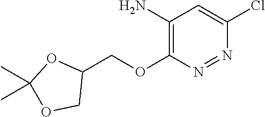 [SMILES: CC1(C)OCC(COc2nnc(Cl)cc2N)O1]

Intermediate 158 was prepared following the procedure used for the synthesis of Intermediate 135, starting from (2,2-dimethyl-1,3-dioxolan-4-yl)methanol (2.42 g, 18.3 mmol) and 3,6-dichloropyridazin-4-amine (1 g, 6.20 mmol) to afford title compound (855 mg, 3.30 mmol, 54% yield). LC-MS (ESI): m/z (M+1): 260.1 (Method 1)

### Intermediate 159: 6-(5-chloro-2-fluorophenyl)-3-[(2,2-dimethyl-1,3-dioxolan-4-yl)methoxy]pyridazin-4-amine

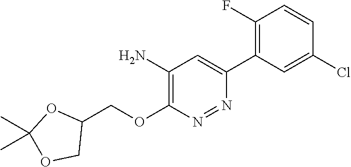 [SMILES: CC1(C)OCC(COc2nnc(-c3cc(Cl)ccc3F)cc2N)O1]

Intermediate 159 was prepared following the procedure used for the synthesis of Intermediate 8, starting from 6-chloro-3-[(2,2-dimethyl-1,3-dioxolan-4-yl)methoxy]pyridazin-4-amine (Intermediate 158, 800 mg, 3.08 mmol) and 5-chloro-2-fluorobenzeneboronic acid (806 mg, 4.62 mmol) in presence of Pd(dppf)Cl₂ (451 mg, 0.62 mmol) to afford title compound (510 mg, 1.44 mmol, 49% yield).

LC-MS (ESI): m/z (M+1): 354.1 (Method 2)

### Intermediate 160: 3-{[(tert-butyldimethylsilyl)oxy]methyl}cyclobutan-1-one

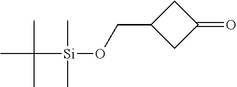

Intermediate 160 was prepared following the procedure used for the synthesis of Intermediate 65 starting from 3-(hydroxymethyl)cyclobutan-1-one (2 g, 20 mmol) to afford title compound (3.6 g, 16.8 mmol, 84% yield).

¹H NMR (400 MHz, Chloroform-d) δ ppm 3.74 (s, 2H), 3.07-3.01 (m, 2H), 2.92 (t, J=3.0 Hz, 2H), 2.65-2.50 (m, 1H), 0.90 (s, 9H), 0.07 (s, 6H).

### Intermediate 161: 3-{[(tert-butyldimethylsilyl)oxy]methyl}cyclobutan-1-ol

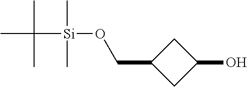

In a flame dried flask, a solution of 3-{[(tert-butyldimethylsilyl)oxy]methyl}cyclobutan-1-one (Intermediate 160, 500 mg, 2.33 mmol) in THE (23.3 mL) was treated with L-Selectride® 1 M in THE (3.5 mL, 3.5 mmol) at −78° C. under N₂ atmosphere. The mixture was stirred for 1 h at −78° C. and then warmed to RT and stirred for 30 minutes. The reaction was quenched by adding 2.5 mL of saturated NaHCO₃ aqueous solution, then cooled using an ice-bath before carefully adding hydrogen peroxide 30% (w/w) in H₂O (0.4 mL, 3.92 mmol). The mixture was warmed to RT and stirred for 15 minutes. The mixture was extracted with EtOAc and washed with water. The organic phase was dried with Na₂SO₄, filtered, and concentrated under reduced pressure. The crude product was purified by flash chromatography on Biotage silica cartridge (from cHex to 25% EtOAc) to afford title compound (472 mg, 2.18 mmol, 93% yield).

¹H NMR (400 MHz, Chloroform-d) δ ppm δ ppm 4.07-4.23 (m, 1H), 3.58 (d, J=4.9 Hz, 2H), 2.31-2.47 (m, 2H), 1.93-2.09 (m, 2H), 1.63-1.78 (m, 2H), 0.86-0.96 (m, 9H), 0.07 (s, 5H).

### Intermediate 162: 3-(3-{[(tert-butyldimethylsilyl)oxy]methyl}cyclobutoxy)-6-chloro-N-[(2,4-dimethoxyphenyl)methyl]pyridazin-4-amine

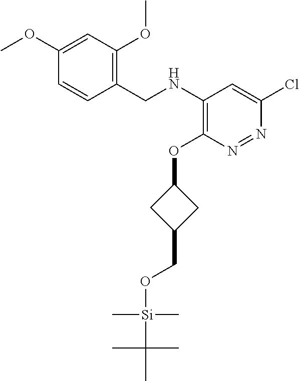

Intermediate 162 was prepared following the procedure used for the synthesis of Intermediate 7 starting from 3,6-dichloro-N-[(2,4-dimethoxyphenyl)methyl]pyridazin-4-amine (Intermediate 6, 400 mg, 1.27 mmol), and 3-{[(tert-butyldimethylsilyl)oxy]methyl}cyclobutan-1-ol (Intermediate 161, 358 mg, 1.65 mmol) at 120° C. to afford title compound (210 mg, 0.42 mmol, 33% yield).

LC-MS (ESI): m/z (M+1): 494.4 (Method 1)

### Intermediate 163: 3-(3-{[(tert-butyldimethylsilyl)oxy]methyl}cyclobutoxy)-6-(5-chloro-2-fluorophenyl)-N-[(2,4-dimethoxyphenyl)methyl]pyridazin-4-amine

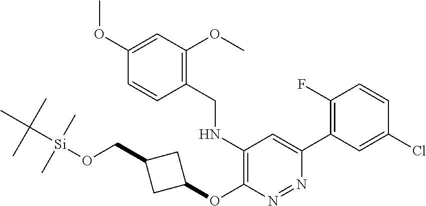

Intermediate 163 was prepared following the procedure used for the synthesis of Intermediate 8, starting from 3-(3-{[(tert-butyldimethylsilyl)oxy]methyl}cyclobutoxy)-6-chloro-N-[(2,4-dimethoxyphenyl)methyl]pyridazin-4-amine (Intermediate 162, 210 mg, 0.43 mmol) and 5-chloro-2-fluorobenzeneboronic acid (111 mg, 0.64 mmol) in presence of Pd(dppf)Cl₂ (62 mg, 0.09 mmol) to afford title compound (110 mg, 0.19 mmol, 44% yield). LC-MS (ESI): m/z (M+1): 588.4 (Method 1)

### Intermediate 164: (3-{[4-amino-6-(5-chloro-2-fluorophenyl)pyridazin-3-yl]oxy}cyclobutyl)methanol

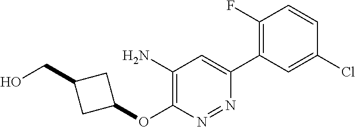

Intermediate 164 was prepared following the procedure used for the synthesis of Intermediate 64, starting from Intermediate 163 (110 mg, 0.19 mmol) to afford title compound (40 mg, 0.12 mmol, 63% yield). LC-MS (ESI): m/z (M+1): 324.1 (Method 1)

### Intermediate 165: 3-{[(4-amino-6-chloropyridazin-3-yl)oxy]methyl}phenol

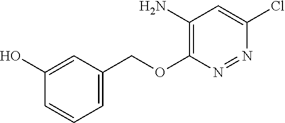 [SMILES: Nc1cc(Cl)nnc1OCc1cccc(O)c1]

To a solution of 3-hydroxybenzyl alcohol (454 mg, 3.66 mmol) in DMF (6.7 mL), NaH 60% dispersion in oil (293 mg, 7.32 mmol) was added and the mixture was stirred at RT for 30 min (until gas evolution ceased). 3,6-dichloropyridazin-4-amine (200 mg, 1.22 mmol) dissolved in DMF (2.7 mL) was added and the reaction warmed at 90° C. for 12 hrs. The mixture was diluted with EtOAc and washed with saturated NaHCO₃ aqueous solution (3×) and brine (1×). The organic phase was filtered through a phase separator and concentrated under vacuum. The residue was taken up with DCM and the resulting precipitate was collected by filtration, affording title compound (100 mg, 0.4 mmol, 33% yield). LC-MS (ESI): m/z (M+1): 252.1 (Method 1)

### Intermediate 166: 3-({[4-amino-6-(5-chloro-2-fluorophenyl)pyridazin-3-yl]oxy}methyl)phenol

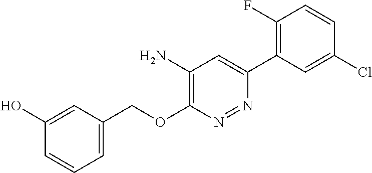 [SMILES: Nc1cc(-c2cc(Cl)ccc2F)nnc1OCc1cccc(O)c1]

Intermediate 166 was prepared following the procedure used for the synthesis of Intermediate 8, starting from 3-{[(4-amino-6-chloropyridazin-3-yl)oxy]methyl}phenol (Intermediate 165, 70 mg, 0.28 mmol) and 5-chloro-2-fluorobenzeneboronic acid (72 mg, 0.42 mmol) in presence of Pd(dppf)Cl₂ (41 mg, 0.06 mmol) to afford title compound (34 mg, 0.10 mmol, 35% yield). LC-MS (ESI): m/z (M+1): 346.1 (Method 2) Intermediate 167: tert-butyl 3-{[(4-bromopyridin-2-yl)carbamoyl]methyl}-3,6-diazabicyclo[3.2.2]nonane-6-carboxylate

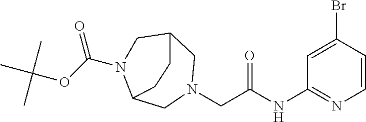 [SMILES: CC(C)(C)OC(=O)N1CC2CCC1CN(CC(=O)Nc1cc(Br)ccn1)C2]

Intermediate 167 was prepared following the procedure used for the synthesis of Intermediate 72, starting from N-(4-bromopyridin-2-yl)-2-chloroacetamide (Intermediate 33, 330 mg, 1.32 mmol) and tert-butyl 3,6-diazabicyclo[3.2.2]nonane-6-carboxylate (359 mg, 1.59 mmol) to afford title compound (460 mg, 1.05 mmol, 79% yield).

LC-MS (ESI): m/z (M+1): 439.2 (Method 1)

### Intermediate 168: N-(4-bromopyridin-2-yl)-2-{3,6-diazabicyclo[3.2.2]nonan-3-yl}acetamide

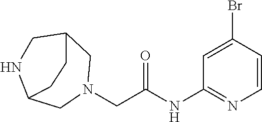 [SMILES: O=C(CN1CC2CCC(C1)NC2)Nc1cc(Br)ccn1]

Intermediate 168 was prepared following the procedure used for the synthesis of Intermediate 40, starting from tert-butyl 3-{[(4-bromopyridin-2-yl)carbamoyl]methyl}-3,6-diazabicyclo[3.2.2]nonane-6-carboxylate (Intermediate 167, 460 mg, 1.05 mmol) to afford title compound (355 mg, 1.05 mmol, quantitative yield).

LC-MS (ESI): m/z (M+1): 339.1 (Method 2)

### Intermediate 169: N-(4-bromopyridin-2-yl)-2-{6-methyl-3,6-diazabicyclo[3.2.2]nonan-3-yl}acetamide

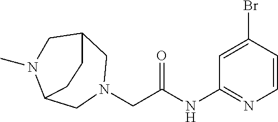 [SMILES: CN1CC2CCC1CN(CC(=O)Nc1cc(Br)ccn1)C2]

Intermediate 169 was prepared following the procedure used for the synthesis of Intermediate 31, starting from N-(4-bromopyridin-2-yl)-2-{3,6-diazabicyclo[3.2.2]nonan-3-yl}acetamide (Intermediate 168, 355 mg, 1.05 mmol) and formaldehyde 37% w/w in water (0.12 mL, 1.57 mmol) to afford title compound (350 mg, 0.99 mmol, 95% yield). LC-MS (ESI): m/z (M+1): 353.1 (Method 2)

### Intermediate 170: ethyl 3-(4-methylpiperazin-1-yl)cyclobutane-1-carboxylate

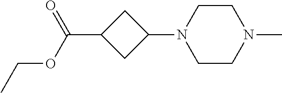 [SMILES: CCOC(=O)C1CC(N2CCN(C)CC2)C1]

1-methylpiperazine (0.55 mL, 5 mmol) and ethyl 3-oxocyclobutane-1-carboxylate (950 mg, 6.7 mmol) were mixed in DCM (30 mL) and stirred for 15 min at RT. Sodium triacetoxyborohydride (2.12 g, 10 mmol) was added portion-wise and the resulting reaction mixture was stirred overnight at RT. MeOH (30 mL) was added carefully and the mixture was stirred for 30 min, then it was concentrated under reduced pressure. The crude material was dissolved in MeOH and the solution was charged on SCX, washed with MeOH, and eluted with 1 N NH₃ in MeOH). Evaporation of basic fractions afforded a crude material that was purified by flash chromatography on Biotage silica NH cartridge (from c-Hex to 30% EtOAc) to afford title compound (927 mg, 4.1 mmol, 82% yield) as inseparable diasteroisomeric mixture cis/trans 9/1 ratio.

LC-MS (ESI): m/z (M+1): 227.3 (Method 2)

### Intermediate 171: N-(4-bromopyridin-2-yl)-3-(4-methylpiperazin-1-yl)cyclobutane-1-carboxamide

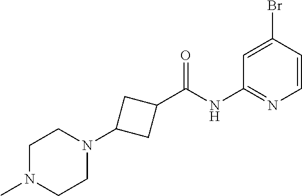 [SMILES: CN1CCN(C2CC(C(=O)Nc3cc(Br)ccn3)C2)CC1]

To a stirred solution of 4-bromopyridin-2-amine (1.15 g, 6.63 mmol) in THE (28 mL), at −78° C. and under a N₂, n-Butyl lithium 1.6 N in hexanes (3.55 mL, 5.68 mmol) was added portion-wise over 10 min then the reaction mixture was stirred at −78° C. for 1 h. A solution of ethyl 3-(4-methylpiperazin-1-yl)cyclobutane-1-carboxylate (Intermediate 170, 600 mg, 2.65 mmol) in THE (12 mL) was added portion-wise over 10 min at −78° C. After 5 min the cooling bath was removed, and the resulting reaction mixture was stirred overnight at RT. The mixture was diluted with MeOH and concentrated under reduced pressure. The crude material was purified by flash chromatography on Biotage silica NH cartridge (from cHex to 40% EtOAc) to afford title compound (410 mg, 1.16 mmol, 44% yield) as inseparable diasteroisomeric mixture cis/trans 9/1 ratio. LC-MS (ESI): m/z (M+1): 355.1 (Method 2)

### Intermediate 172: Cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)cyclobutane-1-carboxamide

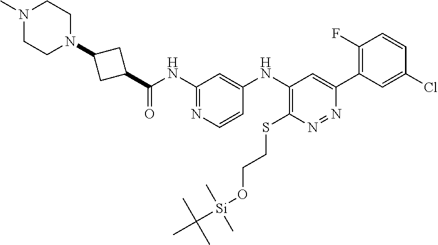

Intermediate 172 was prepared following the procedure used for the synthesis of Intermediate 47 starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and N-(4-bromopyridin-2-yl)-3-(4-methylpiperazin-1-yl)cyclobutane-1-carboxamide (Intermediate 171, 102.7 mg, 0.29 mmol) to afford title compound (60 mg, 0.09 mmol, 36% yield). Only the major isomer cis was isolated.

LC-MS (ESI): m/z (M+1): 686.4 (Method 2)

### Intermediate 173: N-(4-bromopyridin-2-yl)-3-(4-methyl-1,4-diazepan-1-yl)propanamide

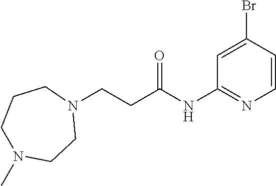 [SMILES: CN1CCCN(CCC(=O)Nc2cc(Br)ccn2)CC1]

Intermediate 173 was prepared following the procedure used for the synthesis of Intermediate 2, starting from N-(4-bromopyridin-2-yl)prop-2-enamide (Intermediate 1, 350 mg, 1.54 mmol) and 1-methyl-1,4-diazepane (238 mg, 2.08 mmol) to afford title compound (434 mg, 1.27 mmol, 82% yield). LC-MS (ESI): m/z (M+1): 341.1 (Method 2)

### Intermediate 174: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methyl-1,4-diazepan-1-yl)propanamide

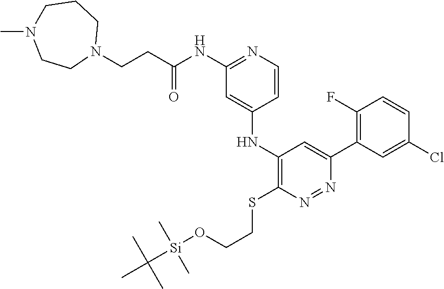 [SMILES: CN1CCCN(CCC(=O)Nc2cc(Nc3cc(-c4cc(Cl)ccc4F)nnc3SCCO[Si](C)(C)C(C)(C)C)ccn2)CC1]

Intermediate 174 was prepared following the procedure used for the synthesis of Intermediate 47 starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and N-(4-bromopyridin-2-yl)-3-(4-methyl-1,4-diazepan-1-yl)propanamide (Intermediate 173, 90.7 mg, 0.27 mmol) to afford title compound (94 mg, 0.14 mmol, 58% yield).

LC-MS (ESI): m/z (M+1): 674.4 (Method 2)

### Intermediate 175: (2,2-dimethyl-1,3-dioxolan-4-yl)methanethiol

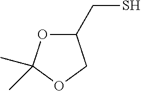

Pyridinium p-toluenesulfonate (232 mg, 0.92 mmol) was added to a stirred mixture of 3-mercapto-1,2-propanediol (1 g, 9.25 mmol) and magnesium sulfate (1.7 g, 13.87 mmol) in acetone (15 mL) at RT. After 3 days the solid was filtered, the solvent was removed by reduced pressure and the residue was purified by flash chromatography on Biotage silica cartridge (from cHex to 10% EtOAc) to afford title compound (650 mg, 4.38 mmol, 47% yield).

¹H NMR (400 MHz, Chloroform-d) δ ppm 4.23 (dq, J=6.70, 5.95 Hz, 1H), 4.13 (dd, J=8.27, 6.10 Hz, 1H), 3.79 (dd, J=8.28, 5.95 Hz, 1H), 2.76 (ddd, J=13.44, 7.92, 5.47 Hz, 1H), 2.63 (ddd, J=13.48, 9.04, 6.72 Hz, 1H), 1.47 (dd, J=11.38, 0.94 Hz, 4H), 1.38 (q, J=0.70 Hz, 3H)

### Intermediate 176: 6-chloro-3-{[(2,2-dimethyl-1,3-dioxolan-4-yl)methyl]sulfanyl}pyridazin-4-amine

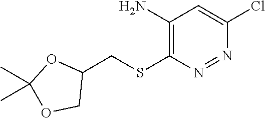 [SMILES: CC1(C)OCC(CSc2nnc(Cl)cc2N)O1]

To an ice-cooled solution of (2,2-dimethyl-1,3-dioxolan-4-yl)methanethiol (Intermediate 175, 678 mg, 4.57 mmol) in DMF (10 mL), NaH 60% dispersion in oil (183 mg, 4.57 mmol) was added and the mixture was stirred at RT for 1 h (until gas evolution ceased). The mixture was cooled with an ice bath, 3,6-dichloropyridazin-4-amine (500 mg, 3.05 mmol) dissolved in DMF (2 mL) was added and the reaction warmed and stirred at RT 3 hrs. The mixture was poured into ice water and extracted with EtOAc. The organic phase was separated, dried over Na₂SO₄, and concentrated under vacuum. The residue was purified by flash chromatography on Biotage silica cartridge (from cHex to 50% EtOAc) to afford title compound (550 mg, 1.99 mmol, 65% yield).

LC-MS (ESI): m/z (M+1): 276.1 (Method 1)

### Intermediate 177: 6-(5-chloro-2-fluorophenyl)-3-{[(2,2-dimethyl-1,3-dioxolan-4-yl)methyl]sulfanyl}pyridazin-4-amine

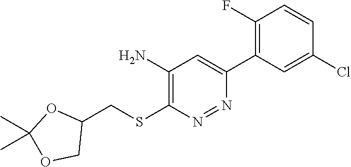 [SMILES: CC1(C)OCC(CSc2nnc(-c3cc(Cl)ccc3F)cc2N)O1]

Intermediate 177 was prepared following the procedure used for the synthesis of Intermediate 8, starting from 6-chloro-3-{[(2,2-dimethyl-1,3-dioxolan-4-yl)methyl]sulfanyl}pyridazin-4-amine (Intermediate 176, 550 mg, 1.99 mmol) and 5-chloro-2-fluorobenzeneboronic acid (522 mg, 2.99 mmol) in presence of Pd(dppf)Cl₂ (146 mg, 1.99 mmol) to afford title compound (220 mg, 0.59 mmol, 30% yield).

LC-MS (ESI): m/z (M+1): 370.1 (Method 2)

### Intermediates 178 and 179: Cis N-(4-bromopyridin-2-yl)-3-[(1S,4S)-5-methyl-2,5-diazabicyclo[2.2.1]heptan-2-yl]cyclobutane-1-carboxamide (178) and trans N-(4-bromopyridin-2-yl)-3-[(1S,4S)-5-methyl-2,5-diazabicyclo[2.2.1]heptan-2-yl]cyclobutane-1-carboxamide (179)

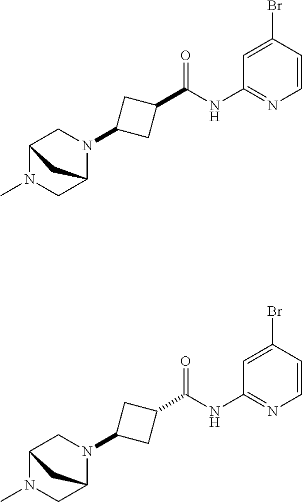

### Step 1

A solution of (1S,4S)-2-methyl-2,5-diazabicyclo[2.2.1]heptane dihydrobromide (1.06 g, 3.87 mmol), DIPEA (1.53 mL, 8.79 mmol) and ethyl 3-oxocyclobutane-1-carboxylate (500 mg, 3.52 mmol) in DCM (15 mL), at RT, was stirred for 15 min then sodium triacetoxyborohydride (1.49 g, 7.03 mmol) was added portion-wise and the resulting reaction mixture was stirred overnight at RT. Methanol (30 mL) was added carefully and the mixture was stirred for 30 min then was concentrated under reduced pressure. The crude material was dissolved in MeOH and charged on SCX washing with MeOH and eluting with 1 N NH₃ in MeOH. Basic fractions were collected, dried and purified by flash chromatography on Biotage silica NH cartridge (from cHex to 15% EtOAc) to afford an inseparable mixture of cis/trans ethyl 3-[(1S,4S)-5-methyl-2,5-diazabicyclo[2.2.1]heptan-2-yl]cyclobutane-1-carboxylate (400 mg, 1.68 mmol, 48% yield) used as such in the next step.

### Step 2

To a stirred solution of 4-bromopyridin-2-amine (0.73 g, 4.2 mmol) in THF (18.7 mL), at −78° C. and under a N₂, n-Butyl lithium 1.6 N in hexanes (1.44 mL, 3.6 mmol) was added portion-wise over 10 min then the reaction mixture was stirred at −78° C. for 1 h. A solution of cis/trans ethyl 3-[(1S,4S)-5-methyl-2,5-diazabicyclo[2.2.1]heptan-2-yl]cyclobutane-1-carboxylate (400 mg, 1.68 mmol) in THE (8 mL) was added portion-wise over 10 min at −78° C. After 5 min the cooling bath was removed, and the resulting reaction mixture was stirred overnight at RT. The mixture was diluted with MeOH and concentrated under reduced pressure. The crude material was purified by flash chromatography on Biotage silica NH cartridge (from cHex to 85% EtOAc, then to 20% MeOH in EtOAc) to afford cis N-(4-bromopyridin-2-yl)-3-[(1S,4S)-5-methyl-2,5-diazabicyclo[2.2.1]heptan-2-yl]cyclobutane-1-carboxamide (180 mg, 0.49 mmol, 29% yield) and trans N-(4-bromopyridin-2-yl)-3-[(1S,4S)-5-methyl-2,5-diazabicyclo[2.2.1]heptan-2-yl]cyclobutane-1-carboxamide (60 mg, 0.16 mmol, 10% yield).

### Intermediate 178: LC-MS (ESI): m/z (M+1): 367.0 (Method 2)

¹H NMR (400 MHz, Chloroform-d) δ ppm 11.80 (br s, 1H), 8.48 (d, J=1.1 Hz, 1H), 8.08 (d, J=5.4 Hz, 1H), 7.15 (dd, J=5.3, 1.5 Hz, 1H), 3.44 (s, 1H), 3.31 (br s, 1H), 3.25-3.31 (m, 1H), 3.08 (tt, J=8.6, 4.5 Hz, 1H), 2.98 (d, J=10.0 Hz, 1H), 2.93 (d, J=10.0 Hz, 1H), 2.69 (dd, J=10.0, 2.4 Hz, 1H), 2.64 (dd, J=9.9, 2.2 Hz, 1H), 2.49-2.63 (m, 2H), 2.46 (s, 3H), 2.06-2.18 (m, 2H), 1.94 (br d, J=9.8 Hz, 1H), 1.77 (br d, J=9.9 Hz, 1H).

### Intermediate 179: LC-MS (ESI): m/z (M+1): 367.0 (Method 2)

¹H NMR (400 MHz, Chloroform-d) δ ppm 8.52 (s, 1H), 8.07 (d, J=5.4 Hz, 1H), 7.81 (br s, 1H), 7.20 (dd, J=5.3, 1.4 Hz, 1H), 3.40 (quin, J=6.7 Hz, 1H), 3.28 (s, 1H), 3.22 (s, 1H), 3.13-3.21 (m, 1H), 2.79 (d, J=9.9 Hz, 1H), 2.62-2.72 (m, 2H), 2.55 (dd, J=10.0, 2.4 Hz, 1H), 2.39-2.52 (m, 2H), 2.38 (s, 3H), 2.14-2.28 (m, 2H), 1.66-1.76 (m, 2H).

### Intermediate 180: Cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-[(1S,4S)-5-methyl-2,5-diazabicyclo[2.2.1]heptan-2-yl]cyclobutane-1-carboxamide

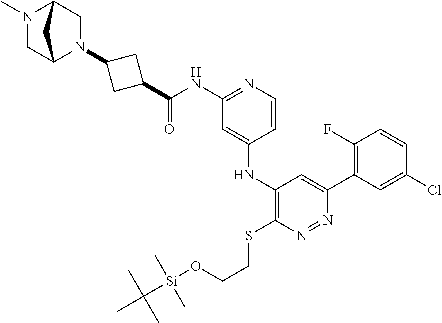

In a microwave vial, a mixture of XantPhos (12 mg, 0.02 mmol), K₃PO₄ (56 mg, 0.27 mmol), 3-({2-[(tert-butyldimethylsilyl)oxy]ethyl}sulfanyl)-6-(5-chloro-2-fluorophenyl)pyridazin-4-amine (Intermediate 67, 55 mg, 0.13 mmol), cis N-(4-bromo-2-pyridyl)-3-[(1S,4S)-5-methyl-2,5-diazabicyclo[2.2.1]heptan-2-yl]cyclobutanecarboxamide (Intermediate 178, 58 mg, 0.16 mmol) and Pd₂(dba)₃ (12 mg, 0.01 mmol) in 1,4-Dioxane (2 mL) was degassed (vacuum/N₂) and heated 5 hrs at 110° C. under microwave irradiation. The mixture was filtered through a Celite® pad washing with EtOAc and the solvent removed under reduced pressure. The crude product was purified by flash chromatography on Biotage silica NH cartridge (cHex/EtOAc/MeOH from 100:0:0 to 0:98:2) to afford title compound (56 mg, 0.08 mmol, 60% yield).

LC-MS (ESI): m/z (M+1): 698.5 (Method 2)

### Intermediate 181: Trans N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-[(1S,4S)-5-methyl-2,5-diazabicyclo[2.2.1]heptan-2-yl]cyclobutane-1-carboxamide

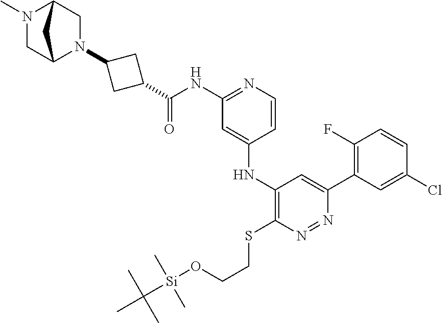

Intermediate 181 was prepared following the procedure used for the synthesis of Intermediate 180 starting from 3-({2-[(tert-butyldimethylsilyl)oxy]ethyl}sulfanyl)-6-(5-chloro-2-fluorophenyl)pyridazin-4-amine (Intermediate 67, 58 mg, 0.14 mmol) and TRANS N-(4-bromopyridin-2-yl)-3-[(1S,4S)-5-methyl-2,5-diazabicyclo[2.2.1]heptan-2-yl]cyclobutane-1-carboxamide (Intermediate 179, 55 mg, 0.15 mmol) to afford title compound (50 mg, 0.07 mmol, 52% yield). LC-MS (ESI): m/z (M+1): 698.5 (Method 2)

### Intermediate 182: ethyl 3-(thiomorpholin-4-yl)cyclobutane-1-carboxylate

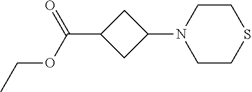

Intermediate 182 was prepared following the procedure used for the synthesis of Intermediate 170 starting from thiomorpholine (0.5 ml, 4.94 mmol) and ethyl 3-oxocyclobutane-1-carboxylate (638 mg, 4.49 mmol) to afford title compound (950 mg, 4.14 mmol, 92% yield) as inseparable diasteroisomeric mixture cis/trans 85/15 ratio.

LC-MS (ESI): m/z (M+1): 230.3 (Method 2)

### Intermediate 183: Cis N-(4-bromopyridin-2-yl)-3-(thiomorpholin-4-yl)cyclobutane-1-carboxamide

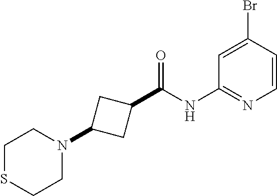

Intermediate 183 was prepared following the procedure used for the synthesis of Intermediate 171 starting from 4-bromopyridin-2-amine (1.79 g, 10.36 mmol) and ethyl 3-(thiomorpholin-4-yl)cyclobutane-1-carboxylate (Intermediate 182, 950 mg, 4.14 mmol) to afford title compound (565 mg, 1.59 mmol, 38% yield). Only the major isomer cis was isolated. LC-MS (ESI): m/z (M+1): 356.0 (Method 2)

### Intermediate 184: Cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(thiomorpholin-4-yl)cyclobutane-1-carboxamide

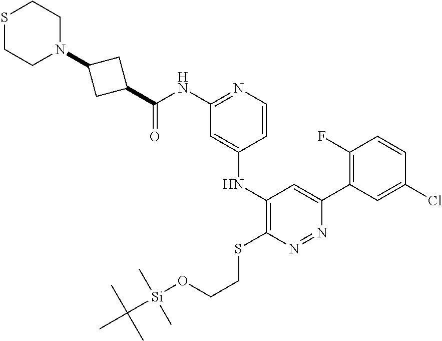

Intermediate 184 was prepared following the procedure used for the synthesis of Intermediate 180 starting from 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol) and cis N-(4-bromopyridin-2-yl)-3-(thiomorpholin-4-yl)cyclobutane-1-carboxamide (Intermediate 183, 103 mg, 0.29 mmol) to afford title compound (140 mg, 0.20 mmol, 84% yield). LC-MS (ESI): m/z (M+1): 689.4 (Method 2)

### Intermediate 185: Cis tert-butyl 7-[3-(ethoxycarbonyl)cyclobutyl]-4,7-diazaspiro[2.5]octane-4-carboxylate

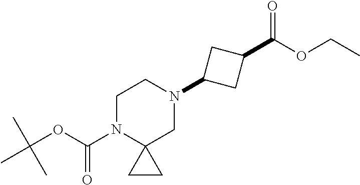

Intermediate 185 was prepared following the procedure used for the synthesis of Intermediate 170 starting from tert-butyl 4,7-diazaspiro[2.5]octane-4-carboxylate (1.15 g, 5.42 mmol) and ethyl 3-oxocyclobutane-1-carboxylate (700 mg, 4.92 mmol) to afford title compound (1.03 g, 3.05 mmol, 62% yield).

LC-MS (ESI): m/z (M+1): 340.0 (Method 2)

¹H NMR (400 MHz, Chloroform-d) δ ppm 4.12 (q, J=7.0 Hz, 2H), 3.55 (br s, 2H), 2.71-2.81 (m, 1H), 2.61-2.70 (m, 1H), 2.30-2.37 (m, 2H), 2.23-2.32 (m, 2H), 2.13-2.17 (m, 2H), 2.05-2.17 (m, 2H), 1.46 (s, 9H), 1.25 (t, J=7.1 Hz, 3H), 0.98 (br s, 2H), 0.74 (s, 2H).

### Intermediate 186: Cis ethyl 3-{4,7-diazaspiro[2.5]octan-7-yl}cyclobutane-1-carboxylate

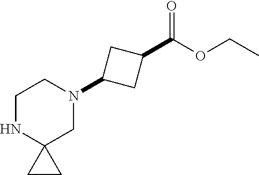

Intermediate 186 was prepared following the procedure used for the synthesis of Intermediate 40 starting from cis tert-butyl 7-[3-(ethoxycarbonyl)cyclobutyl]-4,7-diazaspiro[2.5]octane-4-carboxylate (Intermediate 185, 1.03 g, 3.05 mmol) to afford title compound (724 mg, 3.04 mmol, 99% yield). LC-MS (ESI): m/z (M+1): 239.9 (Method 2)

### Intermediate 187: Cis ethyl 3-{4-methyl-4,7-diazaspiro[2.5]octan-7-yl}cyclobutane-1-carboxylate

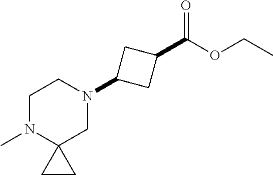

Intermediate 187 was prepared following the procedure used for the synthesis of Intermediate 31 starting from Cis ethyl 3-{4,7-diazaspiro[2.5]octan-7-yl}cyclobutane-1-carboxylate (Intermediate 186, 724 mg, 3.04 mmol) and formaldehyde 37% w/w in water (0.3 mL, 3.95 mmol) to afford title compound (540 mg, 2.14 mmol, 70% yield).

LC-MS (ESI): m/z (M+1): 253.4 (Method 2)

### Intermediate 188: Cis N-(4-bromopyridin-2-yl)-3-{4-methyl-4,7-diazaspiro[2.5]octan-7-yl}cyclobutane-1-carboxamide

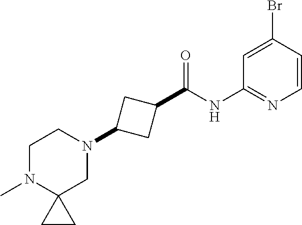

Intermediate 188 was prepared following the procedure used for the synthesis of Intermediate 171 starting from 4-bromopyridin-2-amine (926 mg, 5.35 mmol) and cis ethyl 3-{4-methyl-4,7-diazaspiro[2.5]octan-7-yl}cyclobutane-1-carboxylate (Intermediate 187, 540 mg, 2.14 mmol) to afford title compound (394 mg, 1.04 mmol, 49% yield). LC-MS (ESI): m/z (M+1): 379.3 (Method 2)

### Intermediate 189: Cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-{4-methyl-4,7-diazaspiro[2.5]octan-7-yl}cyclobutane-1-carboxamide

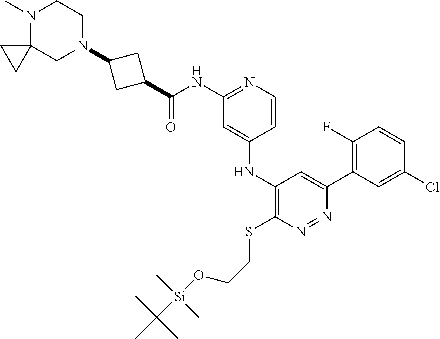

A mixture of XantPhos (36 mg, 0.06 mmol), K₃PO₄ (179 mg, 0.83 mmol), 3-({2-[(tert-butyldimethylsilyl)oxy]ethyl}sulfanyl)-6-(5-chloro-2-fluorophenyl)pyridazin-4-amine (Intermediate 67, 172 mg, 0.42 mmol), cisN-(4-bromopyridin-2-yl)-3-{4-methyl-4,7-diazaspiro[2.5]octan-7-yl}cyclobutane-1-carboxamide (Intermediate 188, 205 mg, 0.54 mmol) and Pd₂(dba)₃ (38 mg, 0.04 mmol) in 1,2-dimethoxyethane (4.15 mL) was degassed (vacuum/N₂) and heated for 45 min at 105° C. The mixture was filtered through a Celite® pad washing with EtOAc and washed with saturated NaHCO₃ aqueous solution, organic solvent was separated, dried and removed under reduced pressure. The crude product was purified by flash chromatography on Biotage silica NH cartridge (from cHex to 100% EtOAc) to afford title compound (79 mg, 0.11 mmol, 27% yield).

LC-MS (ESI): m/z (M+1): 712.4 (Method 2)

### Intermediate 190: Cis N-(6-chloropyrimidin-4-yl)-3-(4-methylpiperazin-1-yl)cyclobutane-1-carboxamide

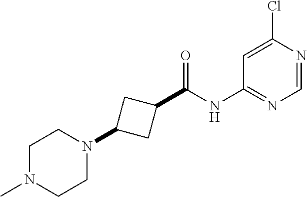

Intermediate 190 was prepared following the procedure used for the synthesis of Intermediate 171 starting from 6-chloro-4-pyrimidinamine (100 mg, 0.77 mmol) and ethyl 3-(4-methylpiperazin-1-yl)cyclobutane-1-carboxylate (Intermediate 170, 192 mg, 0.85 mmol) to afford title compound (19 mg, 0.06 mmol, 7.5% yield).

LC-MS (ESI): m/z (M+1): 310.2 (Method 2)

### Intermediate 191: Cis N-(6-{[3-({2-[(tert-butyldimethylsilyl)oxy]ethyl}sulfanyl)-6-(5-chloro-2-fluorophenyl)pyridazin-4-yl]amino}pyrimidin-4-yl)-3-(4-methylpiperazin-1-yl)cyclobutane-1-carboxamide

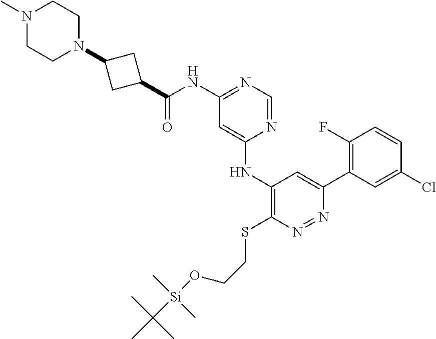

Intermediate 191 was prepared following the procedure used for the synthesis of Intermediate 189 starting from 3-({2-[(tert-butyldimethylsilyl)oxy]ethyl}sulfanyl)-6-(5-chloro-2-fluorophenyl)pyridazin-4-amine (Intermediate 67, 22 mg, 0.05 mmol) and cis N-(6-chloropyrimidin-4-yl)-3-(4-methylpiperazin-1-yl)cyclobutane-1-carboxamide (Intermediate 190, 19 mg, 0.06 mmol) to afford title compound (15 mg, 0.02 mmol, 41% yield).

LC-MS (ESI): m/z (M+1): 687.4 (Method 2)

### Intermediate 192: 4-bromo-3-({2-[(tert-butyldimethylsilyl)oxy]ethyl}sulfanyl)-6-(5-chloro-2-fluorophenyl)pyridazine

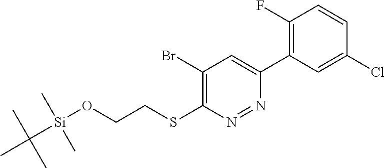 [SMILES: CC(C)(C)[Si](C)(C)OCCSc1nnc(-c2cc(Cl)ccc2F)cc1Br]

A suspension of 3-({2-[(tert-butyldimethylsilyl)oxy]ethyl}sulfanyl)-6-(5-chloro-2-fluorophenyl)pyridazin-4-amine (Intermediate 67, 303 mg, 0.72 mmol), in MeCN (3.6 mL) was treated with copper (II) bromide (274 mg, 1.23 mmol) followed by tert-butyl nitrite (0.15 mL, 1.23 mmol) at RT. The mixture was stirred for 2 hrs and then quenched by adding saturated NaHCO₃ aqueous solution. The mixture was diluted with H₂O and extracted with EtOAc. The organic phase was dried with Na₂SO₄, filtered, and concentrated under reduced pressure. The crude product was purified by flash chromatography on Biotage silica NH cartridge (from cHex to 30% EtOAc) to afford title compound (265 mg, 0.55 mmol, 77% yield). LC-MS (ESI): m/z (M+1): 477.1 (Method 1)

### Intermediate 193: methyl 3-(1-methyl-1,2,3,6-tetrahydropyridin-4-yl)thiophene-2-carboxylate

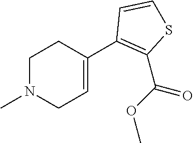 [SMILES: COC(=O)c1sccc1C1=CCN(C)CC1]

In a suitable vial, a mixture of methyl 3-iodothiophene-2-carboxylate (1 g, 3.73 mmol), Pd(dppf)Cl₂ (273 mg, 0.37 mmol), 1-methyl-1,2,3,6-tetrahydropyridine-4-boronic acid pinacol ester (1.08 g, 4.85 mmol) and Na₂CO₃ (791 mg, 7.46 mmol) in 1,2-dimethoxyethane (9.607 mL) and H₂O (4.8 mL) was degassed by bubbling N₂ for 10 minutes then heated at 70° C. for 3 hrs. The mixture was filtered through a pad of Celite® washing with EtOAc. The organic phase was washed with saturated NaHCO₃ aqueous solution and brine, dried with Na₂SO₄, filtered, and concentrated under reduced pressure. The crude product was purified by flash chromatography on Biotage silica cartridge (from DCM to 10% MeOH) to afford title compound (693 mg, 2.92 mmol, 78% yield).

LC-MS (ESI): m/z (M+1): 238.2 (Method 1)

### Intermediate 194: methyl 3-(1-methylpiperidin-4-yl)thiophene-2-carboxylate

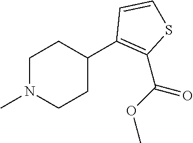 [SMILES: COC(=O)c1sccc1C1CCN(C)CC1]

A mixture of methyl 3-(1-methyl-1,2,3,6-tetrahydropyridin-4-yl)thiophene-2-carboxylate (Intermediate 193, 580 mg, 2.44 mmol) and 5% w/w Pd over carbon (1.18 g, 0.56 mmol) in MeOH (12 mL) was stirred under H₂ atmosphere for 20 hrs. The mixture was filtered over Celite®, the cake washed with MeOH and the solvent removed under reduced pressure. The residue was dissolved again in MeOH (12 mL), treated with 5% w/w Pd over carbon (1.18 g, 0.56 mmol) and stirred under H₂ atmosphere for 4 hrs. The mixture was filtered over Celite®, the cake washed with MeOH and the solvent removed under reduced pressure. The crude product was purified by flash chromatography on Biotage silica cartridge (from DCM to 75% MeOH) to afford title compound (355 mg, 1.48 mmol, 53% yield). LC-MS (ESI): m/z (M+1): 240.2 (Method 1)

### Intermediate 195: methyl 5-iodo-3-(1-methylpiperidin-4-yl)thiophene-2-carboxylate

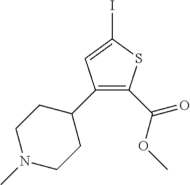 [SMILES: COC(=O)c1sc(I)cc1C1CCN(C)CC1]

Lithium diisopropylamide solution 2.0 M in THE (0.96 mL, 1.93 mmol) was added to a solution of methyl 3-(1-methylpiperidin-4-yl)thiophene-2-carboxylate (Intermediate 194, 317 mg, 1.28 mmol) in THE (8.5 mL) cooled at −78° C. under nitrogen atmosphere. The mixture was stirred for 2 hrs at the same temperature and then treated with solid iodine (489 mg, 1.93 mmol) at −78° C. The reaction was stirred for 1 minute, then warmed to RT and quenched by adding saturated NaHCO₃ aqueous solution followed by saturated Na₂S₂O₃ aqueous solution. The mixture was extracted with DCM, dried with Na₂SO₄, filtered, and concentrated under reduced pressure. The crude product was purified by reverse flash chromatography on Biotage C18 cartridge (from H₂O+0.1% HCOOH to 18% MeCN+0.1% HCOOH). Collected fractions were treated with saturated NaHCO₃ aqueous solution and extracted with EtOAc. The solvent was dried with Na₂SO₄, filtered, and concentrated under reduced pressure to afford title compound (235 mg, 0.64 mmol, 50% yield). LC-MS (ESI): m/z (M+1): 366.1 (Method 1)

### Intermediate 196: methyl 3-(1-methylpiperidin-4-yl)-5-[(4-nitropyridin-2-yl)amino]thiophene-2-carboxylate

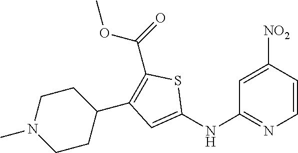 [SMILES: COC(=O)c1sc(Nc2cc([N+](=O)[O-])ccn2)cc1C1CCN(C)CC1]

Intermediate 196 was prepared following the procedure used for the synthesis of Intermediate 189 starting from 2-amino-4-nitropyridine (116 mg, 0.84 mmol) and methyl 5-iodo-3-(1-methylpiperidin-4-yl)thiophene-2-carboxylate (Intermediate 195, 235 mg, 0.64 mmol) to afford title compound (112 mg, 0.30 mmol, 46% yield).

LC-MS (ESI): m/z (M+1): 377.3 (Method 1)

### Intermediate 197: methyl 5-{[(tert-butoxy)carbonyl](4-nitropyridin-2-yl)amino}-3-(1-methylpiperidin-4-yl)thiophene-2-carboxylate

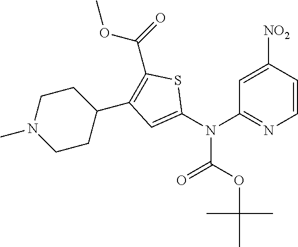 [SMILES: COC(=O)c1sc(N(C(=O)OC(C)(C)C)c2cc([N+](=O)[O-])ccn2)cc1C1CCN(C)CC1]

A solution of DMAP (84 mg, 0.68 mmol) and methyl 3-(1-methylpiperidin-4-yl)-5-[(4-nitropyridin-2-yl)amino]thiophene-2-carboxylate (Intermediate 196, 112 mg, 0.30 mmol) in DCM (3 mL) was treated with di-tert-butyl dicarbonate (156 mg, 0.71 mmol) and stirred at RT overnight. The reaction was quenched by adding saturated NaHCO₃ aqueous solution and extracted with DCM. The organic phase was dried with Na₂SO₄, filtered, and concentrated under reduced pressure. The crude product was purified by reverse flash chromatography on Biotage C18 cartridge (from H₂O+0.1% HCOOH to 40% MeCN+0.1% HCOOH). Collected fractions were treated with saturated NaHCO₃ aqueous solution and extracted with EtOAc. The organic phase was dried with Na₂SO₄, filtered, and concentrated under reduced pressure to afford title compound (88 mg, 0.18 mmol, 62% yield). LC-MS (ESI): m/z (M+1): 477.3 (Method 1)

### Intermediate 198: methyl 5-[(4-aminopyridin-2-yl)[(tert-butoxy)carbonyl]amino]-3-(1-methylpiperidin-4-yl)thiophene-2-carboxylate

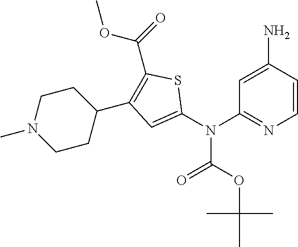 [SMILES: COC(=O)c1sc(N(C(=O)OC(C)(C)C)c2cc(N)ccn2)cc1C1CCN(C)CC1]

A mixture of methyl 5-{[(tert-butoxy)carbonyl](4-nitropyridin-2-yl)amino}-3-(1-methylpiperidin-4-yl)thiophene-2-carboxylate (Intermediate 197, 88 mg, 0.18 mmol) and 5% w/w Pd over carbon (79 mg, 0.04 mmol) in MeOH (1.23 mL) was stirred under H₂ atmosphere overnight. The conversion was partial, so the mixture was filtered over Celite® pad washing with MeOH and concentrated under reduced pressure. The material obtained was dissolved with MeOH (1.73 mL), 10% w/w Pd over carbon (37 mg, 0.03 mmol) and ammonium formate (55 mg, 0.86 mmol) were added and the mixture was stirred at reflux for 1 h. The mixture was filtered over Celite® pad washing with MeOH and the solvent concentrated under reduced pressure. The crude material was purified by flash chromatography on Biotage silica NH cartridge (from DCM to 4% MeOH) to afford title compound (39 mg, 0.09 mmol, 50% yield). LC-MS (ESI): m/z (M+1): 447.2 (Method 1)

### Intermediate 199: methyl 5-{[(tert-butoxy)carbonyl](4-{[3-({2-[(tert-butyldimethylsilyl)oxy]ethyl}sulfanyl)-6-(5-chloro-2-fluorophenyl)pyridazin-4-yl]amino}pyridin-2-yl)amino}-3-(1-methylpiperidin-4-yl)thiophene-2-carboxylate

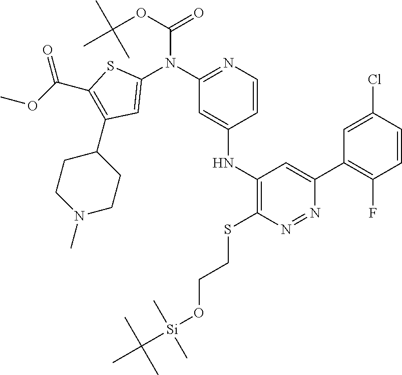 [SMILES: COC(=O)c1sc(N(C(=O)OC(C)(C)C)c2cc(Nc3cc(-c4cc(Cl)ccc4F)nnc3SCCO[Si](C)(C)C(C)(C)C)ccn2)cc1C1CCN(C)CC1]

Intermediate 199 was prepared following the procedure used for the synthesis of Intermediate 180 starting from 4-bromo-3-({2-[(tert-butyldimethylsilyl)oxy]ethyl}sulfanyl)-6-(5-chloro-2-fluorophenyl)pyridazine (Intermediate 192, 41 mg, 0.09 mmol) and methyl 5-[(4-aminopyridin-2-yl)[(tert-butoxy)carbonyl]amino]-3-(1-methylpiperidin-4-yl)thiophene-2-carboxylate (Intermediate 198, 38 mg, 0.09 mmol) to afford title compound (44 mg, 0.05 mmol, 55% yield). LC-MS (ESI): m/z (M+1): 843.5 (Method 2)

### Intermediate 200: 2-[(6-chloro-4-{[(2,4-dimethoxyphenyl)methyl]amino}pyridazin-3-yl)sulfanyl]-2-methylpropan-1-ol

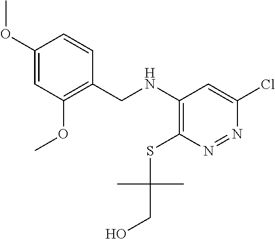 [SMILES: COc1ccc(CNc2cc(Cl)nnc2SC(C)(C)CO)c(OC)c1]

Intermediate 200 was prepared following the procedure used for the synthesis of Intermediate 7 starting from 3,6-dichloro-N-[(2,4-dimethoxyphenyl)methyl]pyridazin-4-amine (Intermediate 6, 700 mg, 2.22 mmol), and 2-methyl-2-sulfanylpropan-1-ol (260 mg, 2.45 mmol) in presence of XantPhos (154 mg, 0.27 mmol) to afford title compound (472 mg, 1.23 mmol, 55% yield). LC-MS (ESI): m/z (M+1): 384.1 (Method 2)

### Intermediate 201: 2-{[6-(5-chloro-2-fluorophenyl)-4-{[(2,4-dimethoxyphenyl)methyl]amino}pyridazin-3-yl]sulfanyl}-2-methylpropan-1-ol

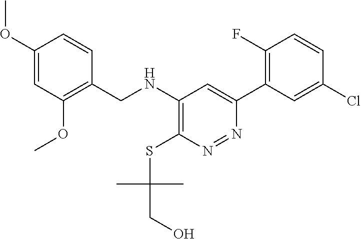 [SMILES: COc1ccc(CNc2cc(-c3cc(Cl)ccc3F)nnc2SC(C)(C)CO)c(OC)c1]

Intermediate 201 was prepared following the procedure used for the synthesis of Intermediate 8, starting from 2-[(6-chloro-4-{[(2,4-dimethoxyphenyl)methyl]amino}pyridazin-3-yl)sulfanyl]-2-methylpropan-1-ol (Intermediate 200, 470 mg, 1.22 mmol) and 5-chloro-2-fluorobenzeneboronic acid (320 mg, 1.84 mmol) in presence of Pd(dppf)Cl₂ (179 mg, 0.24 mmol) to afford title compound (233 mg, 0.49 mmol, 40% yield). LC-MS (ESI): m/z (M+1): 478.2 (Method 2)

### Intermediate 202: 2-{[4-amino-6-(5-chloro-2-fluorophenyl)pyridazin-3-yl]sulfanyl}-2-methylpropan-1-ol

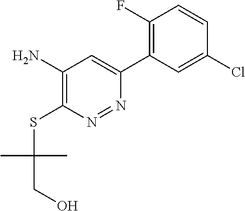 [SMILES: CC(C)(CO)Sc1nnc(-c2cc(Cl)ccc2F)cc1N]

Intermediate 202 was prepared following the procedure used for the synthesis of Intermediate 64, starting from 2-{[6-(5-chloro-2-fluorophenyl)-4-{[(2,4-dimethoxyphenyl)methyl]amino}pyridazin-3-yl]sulfanyl}-2-methylpropan-1-ol (Intermediate 201, 233 mg, 0.49 mmol) to afford title compound (82 mg, 0.25 mmol, 51% yield). LC-MS (ESI): m/z (M+1): 328.1 (Method 2)

### Intermediate 203: 3-({1-[(tert-butyldimethylsilyl)oxy]-2-methylpropan-2-yl}sulfanyl)-6-(5-chloro-2-fluorophenyl)pyridazin-4-amine

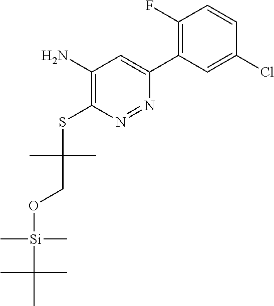 [SMILES: CC(C)(CO[Si](C)(C)C(C)(C)C)Sc1nnc(-c2cc(Cl)ccc2F)cc1N]

Intermediate 203 was prepared following the procedure used for the synthesis of Intermediate 65 starting from 2-{[4-amino-6-(5-chloro-2-fluorophenyl)pyridazin-3-yl]sulfanyl}-2-methylpropan-1-ol (Intermediate 202, 35 mg, 0.11 mmol) to afford title compound (44 mg, 0.10 mmol, 93% yield). LC-MS (ESI): m/z (M+1): 442.2 (Method 2)

### Intermediate 204: N-(4-{[3-({1-[(tert-butyldimethylsilyl)oxy]-2-methylpropan-2-yl}sulfanyl)-6-(5-chloro-2-fluorophenyl)pyridazin-4-yl]amino}pyridin-2-yl)-3-(4-methylpiperazin-1-yl)propanamide

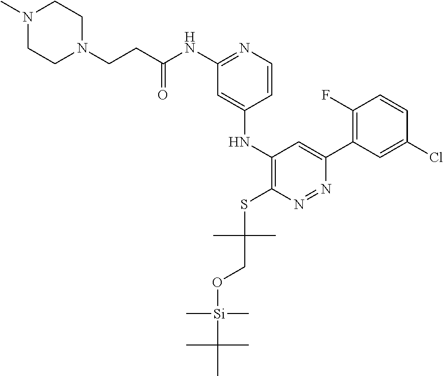 [SMILES: CN1CCN(CCC(=O)Nc2cc(Nc3cc(-c4cc(Cl)ccc4F)nnc3SC(C)(C)CO[Si](C)(C)C(C)(C)C)ccn2)CC1]

Intermediate 204 was prepared following the procedure used for the synthesis of Intermediate 47 starting from 3-({1-[(tert-butyldimethylsilyl)oxy]-2-methylpropan-2-yl}sulfanyl)-6-(5-chloro-2-fluorophenyl)pyridazin-4-amine (Intermediate 203, 44 mg, 0.10 mmol) and N-(4-bromopyridin-2-yl)-3-(4-methylpiperazin-1-yl)propanamide (Intermediate 2, 36 mg, 0.11 mmol) to afford title compound (44 mg, 0.06 mmol, 64% yield). LC-MS (ESI): m/z (M+1): 688.4 (Method 2)

### Intermediate 205: Cis N-(4-{[3-({1-[(tert-butyldimethylsilyl)oxy]-2-methylpropan-2-yl}sulfanyl)-6-(5-chloro-2-fluorophenyl)pyridazin-4-yl]amino}pyridin-2-yl)-3-(4-methylpiperazin-1-yl)cyclobutane-1-carboxamide

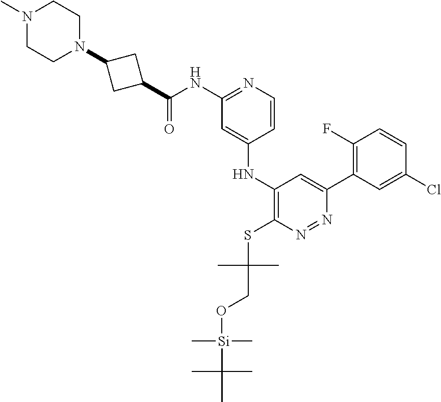

Intermediate 205 was prepared following the procedure used for the synthesis of Intermediate 189 starting from 3-({1-[(tert-butyldimethylsilyl)oxy]-2-methylpropan-2-yl}sulfanyl)-6-(5-chloro-2-fluorophenyl)pyridazin-4-amine (Intermediate 203, 60 mg, 0.14 mmol) and N-(4-bromopyridin-2-yl)-3-(4-methylpiperazin-1-yl)cyclobutane-1-carboxamide (Intermediate 171, 56 mg, 0.15 mmol) to afford title compound (60 mg, 0.08 mmol, 61% yield). Only the major isomer cis was isolated.

LC-MS (ESI): m/z (M+1): 714.4 (Method 2)

### Intermediate 206: methyl 6-chloro-3-[3-(hydroxymethyl)azetidin-1-yl]pyridazine-4-carboxylate

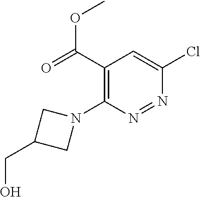 [SMILES: COC(=O)c1cc(Cl)nnc1N1CC(CO)C1]

Intermediate 206 was prepared following the procedure used for the synthesis of Intermediate 94 starting from methyl 3,6-dichloropyridazine-4-carboxylate (Intermediate 93, 100 mg, 0.48 mmol) and (azetidin-3-yl)methanol hydrochloride (60 mg, 0.48 mmol) to afford title compound (80 mg, 0.31 mmol, 64% yield).

LC-MS (ESI): m/z (M+1): 258.2 (Method 1)

### Intermediate 207: methyl 6-(5-chloro-2-fluorophenyl)-3-[3-(hydroxymethyl)azetidin-1-yl]pyridazine-4-carboxylate

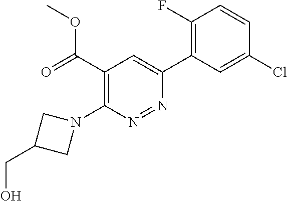 [SMILES: COC(=O)c1cc(-c2cc(Cl)ccc2F)nnc1N1CC(CO)C1]

Intermediate 207 was prepared following the procedure used for the synthesis of Intermediate 8, starting from methyl 6-chloro-3-[3-(hydroxymethyl)azetidin-1-yl]pyridazine-4-carboxylate (Intermediate 206, 80 mg, 0.31 mmol) and 5-chloro-2-fluorobenzeneboronic acid (108 mg, 0.62 mmol) in presence of Pd(dppf)Cl₂ (45 mg, 0.06 mmol) to afford title compound (90 mg, 0.26 mmol, 82% yield).

LC-MS (ESI): m/z (M+1): 352.1 (Method 1)

### Intermediate 208: methyl 3-(3-{[(tert-butyldimethylsilyl)oxy]methyl}azetidin-1-yl)-6-(5-chloro-2-fluorophenyl)pyridazine-4-carboxylate

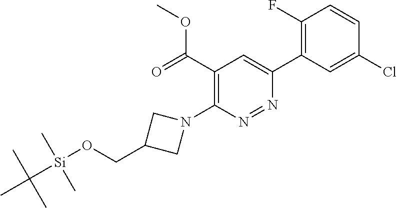 [SMILES: COC(=O)c1cc(-c2cc(Cl)ccc2F)nnc1N1CC(CO[Si](C)(C)C(C)(C)C)C1]

Intermediate 208 was prepared following the procedure used for the synthesis of Intermediate 65 starting from methyl 6-(5-chloro-2-fluorophenyl)-3-[3-(hydroxymethyl)azetidin-1-yl]pyridazine-4-carboxylate (Intermediate 207, 90 mg, 0.26 mmol) to afford title compound (105 mg, 0.22 mmol, 88% yield).

LC-MS (ESI): m/z (M+1): 466.3 (Method 1)

### Intermediate 209: 3-(3-{[(tert-butyldimethylsilyl)oxy]methyl}azetidin-1-yl)-6-(5-chloro-2-fluorophenyl)pyridazine-4-carboxylic acid

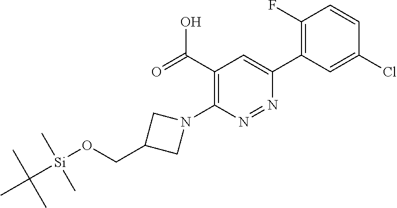 [SMILES: CC(C)(C)[Si](C)(C)OCC1CN(c2nnc(-c3cc(Cl)ccc3F)cc2C(=O)O)C1]

Intermediate 209 was prepared following the procedure used for the synthesis of Intermediate 96 starting from methyl 3-(3-{[(tert-butyldimethylsilyl)oxy]methyl}azetidin-1-yl)-6-(5-chloro-2-fluorophenyl)pyridazine-4-carboxylate (Intermediate 208, 105 mg, 0.22 mmol) to afford title compound (90 mg, 0.20 mmol, 90% yield). LC-MS (ESI): m/z (M+1): 452.2 (Method 1)

### Intermediate 210: 3-(3-{[(tert-butyldimethylsilyl)oxy]methyl}azetidin-1-yl)-6-(5-chloro-2-fluorophenyl)pyridazin-4-amine

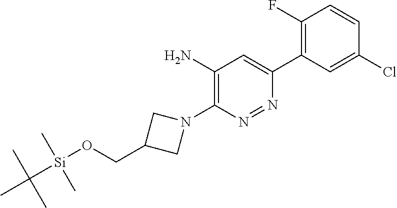 [SMILES: CC(C)(C)[Si](C)(C)OCC1CN(c2nnc(-c3cc(Cl)ccc3F)cc2N)C1]

Intermediate 210 was prepared following the procedure used for the synthesis of Intermediate 97 (Method B) starting from 3-(3-{[(tert-butyldimethylsilyl)oxy]methyl}azetidin-1-yl)-6-(5-chloro-2-fluorophenyl)pyridazine-4-carboxylic acid (Intermediate 209, 70 mg, 0.15 mmol) to afford title compound (23 mg, 0.05 mmol, 35% yield). LC-MS (ESI): m/z (M+1): 452.2 (Method 1)

### Intermediate 211: N-(4-{[3-(3-{[(tert-butyldimethylsilyl)oxy]methyl}azetidin-1-yl)-6-(5-chloro-2-fluorophenyl)pyridazin-4-yl]amino}pyridin-2-yl)-3-(4-methylpiperazin-1-yl)propanamide

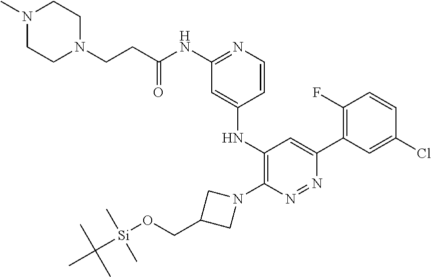 [SMILES: CN1CCN(CCC(=O)Nc2cc(Nc3cc(-c4cc(Cl)ccc4F)nnc3N3CC(CO[Si](C)(C)C(C)(C)C)C3)ccn2)CC1]

Intermediate 211 was prepared following the procedure used for the synthesis of Intermediate 47 starting from 3-(3-{[(tert-butyldimethylsilyl)oxy]methyl}azetidin-1-yl)-6-(5-chloro-2-fluorophenyl)pyridazin-4-amine (Intermediate 210, 38 mg, 0.07 mmol) and N-(4-bromopyridin-2-yl)-3-(4-methylpiperazin-1-yl)propanamide (Intermediate 2, 28 mg, 0.08 mmol) to afford title compound (30 mg, 0.04 mmol, 66% yield).

LC-MS (ESI): m/z (M+1): 669.6 (Method 1).

### Intermediate 212: tert-butyl 3,6-dichloropyridazine-4-carboxylate

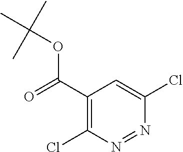

### Method A

A solution of 3,6-dichloro-4-pyridazinecarboxylic acid (4.5 g, 23.32 mmol), DMAP (1.64 g, 13.48 mmol) and 2-methyl-2-propanol (4.14 mL, 43.39 mmol) in DCM (93 mL) was treated with N,N′-Dicyclohexylcarbodiimide (11.76 g, 57 mmol) at RT and stirred for 24 hrs. The mixture was filtered over Celite® pad, the organic phase was concentrated under reduced pressure. The residue was taken-up in DCM and filtered using a phase separator. The organic phase was washed with saturated NaHCO₃ aqueous solution, with 0.1 M aqueous HCl, and H₂O. The organic phase was dried with Na₂SO₄, filtered, and concentrated under reduced pressure. The residue was purified by flash chromatography on Biotage silica cartridge (from cHex to 5% EtOAc) and then by reverse flash chromatography on Biotage C18 cartridge (from H₂O+0.1% HCOOH to 65% MeCN +0.1% HCOOH). Collected fractions were treated with saturated NaHCO₃ aqueous solution and extracted with DCM. The organic phase was dried with Na₂SO₄, filtered, and concentrated under reduced pressure to afford title compound (2.93 g, 11.76 mmol, 57% yield).

### Method B

3,6-dichloro-4-pyridazinecarboxylic acid (500 mg, 2.59 mmol), DMAP (158 mg, 1.3 mmol), and di-tert-butyl dicarbonate (650 mg, 2.98 mmol) were suspended in THE (12 mL) and heated at 65° C. until gas evolution ceased (45 min). Solvent was removed under vacuum, the residue was dissolved with EtOAc, then washed with 5% aqueous HCl (2×), 5% aqueous NaOH and brine. Organic solvent was dried and evaporated to afford title compound (520 mg, 2.09 mmol, 81% yield). LC-MS (ESI): m/z (M+1): 249.1 (Method 1)

### Intermediate 213: tert-butyl 6-chloro-3-[3-(methoxycarbonyl)azetidin-1-yl]pyridazine-4-carboxylate

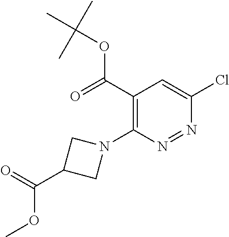 [SMILES: COC(=O)C1CN(c2nnc(Cl)cc2C(=O)OC(C)(C)C)C1]

Intermediate 213 was prepared following the procedure used for the synthesis of Intermediate 94 starting from tert-butyl 3,6-dichloropyridazine-4-carboxylate (Intermediate 212, 350 mg, 1.41 mmol) and methyl azetidine-3-carboxylate hydrochloride (213 mg, 1.04 mmol) to afford title compound (312 mg, 0.95 mmol, 68% yield). LC-MS (ESI): m/z (M+1): 328.2 (Method 1)

### Intermediate 214: tert-butyl 6-(5-chloro-2-fluorophenyl)-3-[3-(methoxycarbonyl)azetidin-1-yl]pyridazine-4-carboxylate

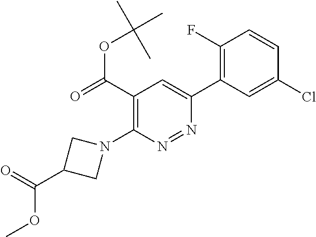 [SMILES: COC(=O)C1CN(c2nnc(-c3cc(Cl)ccc3F)cc2C(=O)OC(C)(C)C)C1]

Intermediate 214 was prepared following the procedure used for the synthesis of Intermediate 8, starting from tert-butyl 6-chloro-3-[3-(methoxycarbonyl)azetidin-1-yl]pyridazine-4-carboxylate (Intermediate 213, 312 mg, 0.95 mmol) and 5-chloro-2-fluorobenzeneboronic acid (249 mg, 1.43 mmol) in presence of Pd(dppf)Cl₂ (70 mg, 0.10 mmol) to afford title compound (320 mg, 0.76 mmol, 80% yield).

LC-MS (ESI): m/z (M+1): 422.3 (Method 1)

### Intermediate 215: 6-(5-chloro-2-fluorophenyl)-3-[3-(methoxycarbonyl)azetidin-1-yl]pyridazine-4-carboxylic acid trifluoroacetic acid salt

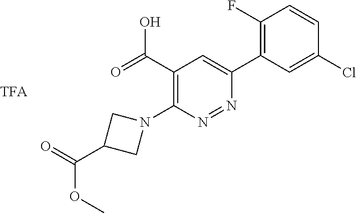 [SMILES: COC(=O)C1CN(c2nnc(-c3cc(Cl)ccc3F)cc2C(=O)O)C1]

A mixture of tert-butyl 6-(5-chloro-2-fluorophenyl)-3-[3-(methoxycarbonyl)azetidin-1-yl]pyridazine-4-carboxylate (Intermediate 214, 320 mg, 0.76 mmol) in DCM (12 mL) and TFA (3 mL) was stirred at RT overnight. Toluene (2 mL) was added to the mixture which was evaporated to dryness to afford title compound (336 mg, 0.70 mmol, 92% yield) as trifluoroacetic salt.

LC-MS (ESI): m/z (M+1): 366.2 (Method 1)

### Intermediate 216: methyl 1-[4-amino-6-(5-chloro-2-fluorophenyl)pyridazin-3-yl]azetidine-3-carboxylate

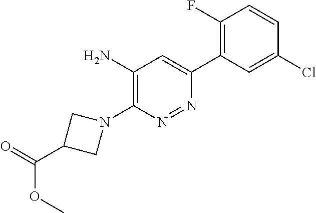 [SMILES: COC(=O)C1CN(c2nnc(-c3cc(Cl)ccc3F)cc2N)C1]

Intermediate 216 was prepared following the procedure used for the synthesis of Intermediate 97 (Method B) starting from 6-(5-chloro-2-fluorophenyl)-3-[3-(methoxycarbonyl)azetidin-1-yl]pyridazine-4-carboxylic acid trifluoroacetic acid salt (Intermediate 215, 336 mg, 0.70 mmol) in presence of TEA (0.21 mL, 1.54 mmol) to afford title compound (124 mg, 0.37 mmol, 53% yield).

LC-MS (ESI): m/z (M+1): 337.1 (Method 1)

### Intermediate 217: N-(4-nitropyridin-2-yl)prop-2-enamide

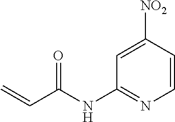

To an ice-cooled solution of 4-nitropyridin-2-amine (1.2 g, 8.63 mmol) in dry DCM (50 mL), TEA (3.6 mL, 25.83 mmol) and 2-propenoyl chloride (1.05 mL, 13 mmol) were added. The solution was stirred at 0° C. for 30 min, then it was allowed to reach RT and stirred at overnight. Water was added, the phases were separated, and the organic phase was dried and evaporated under vacuum. The crude material was purified by flash chromatography on Biotage silica cartridge (from cHex to 32% EtOAc) to afford title compound (847 mg, 4.38 mmol, 51% yield). LC-MS (ESI): m/z (M+1): 194.0 (Method 1)

### Intermediate 218: 3-(4-methylpiperazin-1-yl)-N-(4-nitropyridin-2-yl)propanamide

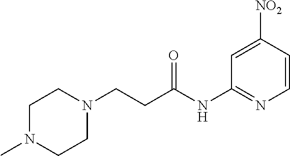 [SMILES: CN1CCN(CCC(=O)Nc2cc([N+](=O)[O-])ccn2)CC1]

Intermediate 218 was prepared following the procedure used for the synthesis of Intermediate 2 starting from N-(4-nitropyridin-2-yl)prop-2-enamide (Intermediate 217, 500 mg, 2.59 mmol) and 1-methylpiperazine (0.65 mL, 5.86 mmol) to afford title compound (674 mg, 2.30 mmol, 89% yield). LC-MS (ESI): m/z (M+1): 294.2 (Method 2)

### Intermediate 219: N-(4-aminopyridin-2-yl)-3-(4-methylpiperazin-1-yl)propanamide

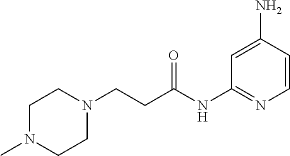 [SMILES: CN1CCN(CCC(=O)Nc2cc(N)ccn2)CC1]

### Method A

A mixture of 3-(4-methylpiperazin-1-yl)-N-(4-nitropyridin-2-yl)propanamide (Intermediate 218, 674 mg, 2.3 mmol) and 10% w/w Pd over carbon (100 mg, 0.94 mmol) in MeOH (60 mL) was stirred under H₂ atmosphere for 6 hrs. The mixture was filtered on Celite® and the filtrate was concentrated under reduced pressure (˜25 mL). 10% w/w Pd over carbon (150 mg, 1.41 mmol) was added and the mixture was stirred under H₂ atmosphere for additional 5 hrs. The mixture was filtered over Celite® pad, and the filtrate was concentrated under reduced pressure. The crude material was purified by flash chromatography on Biotage silica NH cartridge (from DCM to 4% MeOH) to afford title compound (120 mg, 0.46 mmol, 20% yield).

### Method B

10% w/w Pd over carbon (117 mg, 0.11 mmol) was added to a stirred mixture of 3-(4-methylpiperazin-1-yl)-N-(4-nitropyridin-2-yl)propanamide (Intermediate 218, 945 mg, 3.22 mmol) and ammonium formate (1.04 g, 16.28 mmol) in ethanol (32 mL). The mixture was stirred at reflux for 45 min. The mixture was filtered over a Celite® pad, the cake was washed with MeOH and the filtrate was concentrated under reduced pressure. The crude material was purified by flash chromatography on Biotage silica NH cartridge (from DCM to 4% MeOH) to afford title compound (284 mg, 1.08 mmol, 36% yield).

LC-MS (ESI): m/z (M+1): 264.2 (Method 2)

### Intermediate 220: 4-bromo-6-chloro-N-[(3-methoxyphenyl)methyl]pyridazin-3-amine

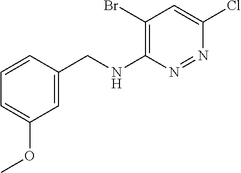 [SMILES: COc1cccc(CNc2nnc(Cl)cc2Br)c1]

To a stirred solution of 3-amino-4-bromo-6-chloropyridazine (500 mg, 2.4 mmol) in THE (7 mL), at 0° C. and under N₂, NaH 60% dispersion in oil (110 mg, 2.75 mmol) was added portion-wise. After 5 min the ice-bath was removed, and the mixture was stirred at RT for 30 min. 1-(bromomethyl)-3-methoxybenzene (0.35 mL, 2.52 mmol) was added drop-wise then the resulting reaction mixture was stirred at 40° C. for 6 hrs. The reaction mixture was concentrated under reduced pressure and the crude material was purified by flash chromatography on Biotage silica cartridge (from cHex to 25% EtOAc) to afford title compound (352 mg, 1.07 mmol, 45% yield).

LC-MS (ESI): m/z (M+1): 328.0 (Method 2)

### Intermediate 221: N-{4-[(6-chloro-3-{[(3-methoxyphenyl)methyl]amino}pyridazin-4-yl)amino]pyridin-2-yl}-3-(4-methylpiperazin-1-yl)propanamide

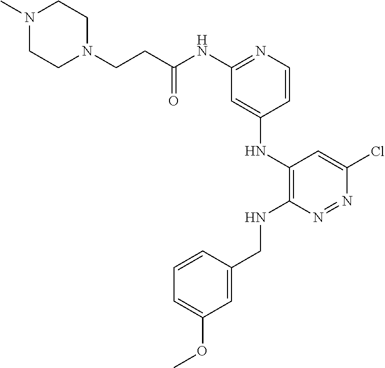 [SMILES: COc1cccc(CNc2nnc(Cl)cc2Nc2ccnc(NC(=O)CCN3CCN(C)CC3)c2)c1]

Intermediate 221 was prepared following the procedure used for the synthesis of Intermediate 189 starting from N-(4-aminopyridin-2-yl)-3-(4-methylpiperazin-1-yl)propanamide (Intermediate 219, 134 mg, 0.51 mmol) and 4-bromo-6-chloro-N-[(3-methoxyphenyl)methyl]pyridazin-3-amine (Intermediate 220, 168 mg, 0.51 mmol) at 120° C. to afford title compound (110 mg, 0.21 mmol, 42% yield).

LC-MS (ESI): m/z (M+1): 511.3 (Method 2)

### Intermediate 222: N-(4-{[6-(5-chloro-2-fluorophenyl)-3-{[(3-methoxyphenyl)methyl]amino}pyridazin-4-yl]amino}pyridin-2-yl)-3-(4-methylpiperazin-1-yl)propanamide

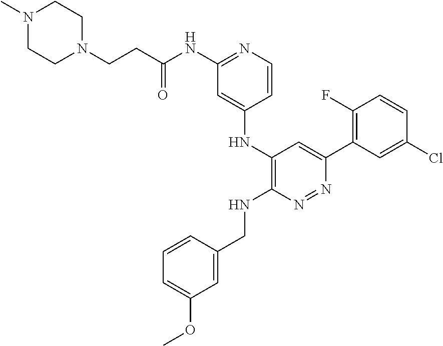 [SMILES: COc1cccc(CNc2nnc(-c3cc(Cl)ccc3F)cc2Nc2ccnc(NC(=O)CCN3CCN(C)CC3)c2)c1]

Intermediate 222 was prepared following the procedure used for the synthesis of Intermediate 8, starting from N-{4-[(6-chloro-3-{[(3-methoxyphenyl)methyl]amino}pyridazin-4-yl)amino]pyridin-2-yl}-3-(4-methylpiperazin-1-yl)propanamide (Intermediate 221, 103 mg, 0.20 mmol) and 5-chloro-2-fluorobenzeneboronic acid (43 mg, 0.25 mmol) in presence of Pd(dppf)Cl₂ (30 mg, 0.04 mmol) to afford title compound (83 mg, 0.14 mmol, 68% yield).

LC-MS (ESI): m/z (M−1): 603.4 (Method 1)

### Intermediate 223: 4-bromo-6-chloro-N-[(3-methoxyphenyl)methyl]-N-methylpyridazin-3-amine

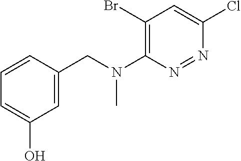 [SMILES: CN(Cc1cccc(O)c1)c1nnc(Cl)cc1Br]

NaH 60% dispersion in oil (52 mg, 1.3 mmol) was added portion-wise to an ice-cooled stirred solution of 4-bromo-6-chloro-N-[(3-methoxyphenyl)methyl]pyridazin-3-amine (Intermediate 220, 350 mg, 1.07 mmol) in THE (5 mL), and under N₂. After 2 min the ice-bath was removed and the mixture was stirred at RT for 25 min, then iodomethane (0.2 mL, 3.21 mmol) was added drop-wise and the resulting reaction mixture was stirred for 5 hrs at 40° C. The reaction mixture was concentrated under reduced pressure and the residue was diluted with DCM, solids were filtered off, and the solution was concentrated under reduced pressure. The crude material was purified by flash chromatography on Biotage silica cartridge (from cHex to 18% EtOAc) to afford title compound (222 mg, 0.65 mmol, 61% yield). LC-MS (ESI): m/z (M−1): 344.0 (Method 1)

### Intermediate 224: N-{4-[(6-chloro-3-{[(3-methoxyphenyl)methyl](methyl)amino}pyridazin-4-yl)amino]pyridin-2-yl}-3-(4-methylpiperazin-1-yl)propanamide

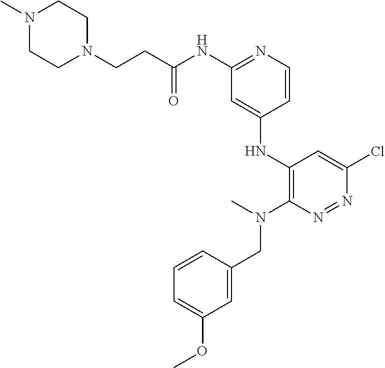 [SMILES: COc1cccc(CN(C)c2nnc(Cl)cc2Nc2ccnc(NC(=O)CCN3CCN(C)CC3)c2)c1]

Intermediate 224 was prepared following the procedure used for the synthesis of Intermediate 189 starting from N-(4-aminopyridin-2-yl)-3-(4-methylpiperazin-1-yl)propanamide (Intermediate 219, 142 mg, 0.54 mmol) and 4-bromo-6-chloro-N-[(3-methoxyphenyl)methyl]-N-methylpyridazin-3-amine (Intermediate 223, 212 mg, 0.62 mmol) at 120° C. to afford title compound (186 mg, 0.35 mmol, 66% yield).

LC-MS (ESI): m/z (M+1): 525.4 (Method 2)

### Intermediate 225: N-(4-{[6-(5-chloro-2-fluorophenyl)-3-{[(3-methoxyphenyl)methyl](methyl)amino}pyridazin-4-yl]amino}pyridin-2-yl)-3-(4-methylpiperazin-1-yl)propanamide

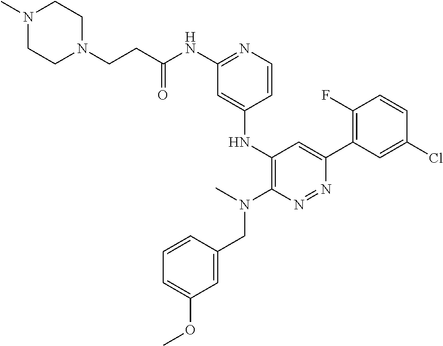 [SMILES: COc1cccc(CN(C)c2nnc(-c3cc(Cl)ccc3F)cc2Nc2ccnc(NC(=O)CCN3CCN(C)CC3)c2)c1]

Intermediate 225 was prepared following the procedure used for the synthesis of Intermediate 8, starting from Intermediate 224 (186 mg, 0.35 mmol) and 5-chloro-2-fluorobenzeneboronic acid (77 mg, 0.44 mmol) in presence of Pd(dppf)Cl₂ (53 mg, 0.07 mmol) to afford title compound (130 mg, 0.21 mmol, 58% yield).

LC-MS (ESI): m/z (M+1): 619.4 (Method 2)

### Intermediate 226: tert-butyl 7-oxo-6-oxa-2-azaspiro[3.4]octane-2-carboxylate

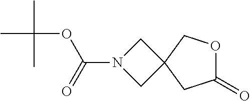 [SMILES: CC(C)(C)OC(=O)N1CC2(COC(=O)C2)C1]

A mixture of tert-butyl 6-oxo-2-azaspiro[3.3]heptane-2-carboxylate (1.5 g, 7.1 mmol) and NaHCO₃ (716 mg, 8.52 mmol) in DCM (35.5 mL) was treated with 3-chloroperbenzoic acid (1.75 g, 7.81 mmol) and stirred at RT overnight. The mixture was quenched by adding saturated Na₂S₂O₃ aqueous solution and saturated NaHCO₃ aqueous solution and extracted with DCM. The solvent was dried with Na₂SO₄, filtered, and concentrated under reduced pressure. The crude product was purified by flash chromatography on Biotage silica NH cartridge (from cHex to 100% EtOAc) to afford title compound (1.55 g, 6.82 mmol, 96% yield).

¹H NMR (500 MHz, Chloroform-d) δ ppm 4.42 (s, 2H), 3.97 (q, J=9.1 Hz, 4H), 2.77 (s, 2H), 1.45 (s, 9H).

### Intermediate 227: 6-oxa-2-azaspiro[3.4]octan-7-one trifluoroacetic acid salt

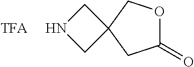

Intermediate 227 was prepared following the procedure used for the synthesis of Intermediate 40 starting from tert-butyl 7-oxo-6-oxa-2-azaspiro[3.4]octane-2-carboxylate (Intermediate 226, 1.55 g, 6.82 mmol) to afford title compound (2.34 g, recovery assumed quantitative).

¹H NMR (400 MHz, DMSO-d₆) δ ppm 8.64 (s, 2H), 4.45 (s, 2H), 4.04 (ddd, J=7.0, 5.6, 1.5 Hz, 4H), 2.91 (s, 2H).

### Intermediate 228: tert-butyl 6-chloro-3-{7-oxo-6-oxa-2-azaspiro[3.4]octan-2-yl}pyridazine-4-carboxylate

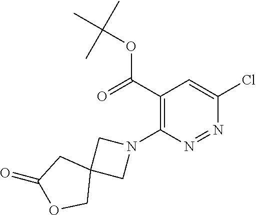 [SMILES: CC(C)(C)OC(=O)c1cc(Cl)nnc1N1CC2(COC(=O)C2)C1]

Intermediate 228 was prepared following the procedure used for the synthesis of Intermediate 94 starting from Intermediate 212 (760 mg, 3.05 mmol) and Intermediate 227 (4.59 mmol) at 30° C. to afford title compound (537 mg, 1.58 mmol, 34% yield). LC-MS (ESI): m/z (M+1): 340.1 (Method 1)

### Intermediate 229: tert-butyl 6-(5-chloro-2-fluorophenyl)-3-{7-oxo-6-oxa-2-azaspiro[3.4]octan-2-yl}pyridazine-4-carboxylate

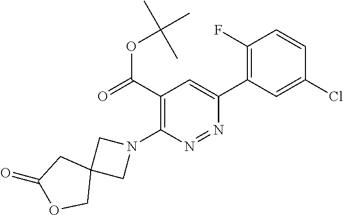 [SMILES: CC(C)(C)OC(=O)c1cc(-c2cc(Cl)ccc2F)nnc1N1CC2(COC(=O)C2)C1]

Intermediate 229 was prepared following the procedure used for the synthesis of Intermediate 16 starting from tert-butyl 6-chloro-3-{7-oxo-6-oxa-2-azaspiro[3.4]octan-2-yl}pyridazine-4-carboxylate (Intermediate 228, 537 mg, 1.58 mmol) and 5-chloro-2-fluorobenzeneboronic acid (965 mg, 5.53 mmol) at 100° C. to afford title compound (343 mg, 0.79 mmol, 50% yield). LC-MS (ESI): m/z (M+1): 434.3 (Method 1) Intermediate 230: 6-(5-chloro-2-fluorophenyl)-3-{7-oxo-6-oxa-2-azaspiro[3.4]octan-2-yl}pyridazine-4-carboxylic acid trifluoroacetic acid salt

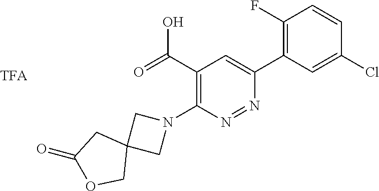 [SMILES: O=C1CC2(CO1)CN(c1nnc(-c3cc(Cl)ccc3F)cc1C(=O)O)C2]

Intermediate 230 was prepared following the procedure used for the synthesis of Intermediate 215 starting from tert-butyl 6-(5-chloro-2-fluorophenyl)-3-{7-oxo-6-oxa-2-azaspiro[3.4]octan-2-yl}pyridazine-4-carboxylate (Intermediate 229, 310 mg, 0.71 mmol) to afford title compound (335 mg, 0.68 mmol, 95% yield) as trifluoroacetic salt.

LC-MS (ESI): m/z (M+1): 378.1 (Method 1)

### Intermediate 231: 2-[4-amino-6-(5-chloro-2-fluorophenyl)pyridazin-3-yl]-6-oxa-2-azaspiro[3.4]octan-7-one

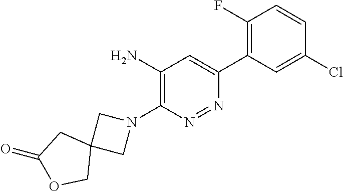 [SMILES: Nc1cc(-c2cc(Cl)ccc2F)nnc1N1CC2(COC(=O)C2)C1]

A solution of Intermediate 230 (335 mg, 0.68 mmol) and TEA (0.3 mL, 2.18 mmol) in tert-butanol (4.54 mL) was treated with diphenyl phosphoryl azide (0.19 mL, 0.89 mmol). The mixture was stirred at 60° C. for 5 hrs. The mixture was diluted with EtOAc, washed with saturated NaHCO₃ aqueous solution and brine. The organic phase was dried with Na₂SO₄, filtered, and concentrated under reduced pressure. This material was dissolved in DCM (4.54 mL), TFA (1.56 mL, 20.43 mmol) was added, and the mixture stirred at RT overnight. The mixture was concentrated under reduced pressure, and then diluted with DCM. The organic phase was washed with saturated NaHCO₃ aqueous solution, dried with Na₂SO₄, filtered, and concentrated under reduced pressure. The crude material was purified by flash chromatography on Biotage silica NH cartridge (from c-Hex to 75% EtOAc) to afford title compound (86 mg, 0.25 mmol, 36% yield).

LC-MS (ESI): m/z (M+1): 349.1 (Method 2)

### Intermediate 232: tert-butyl 6-chloro-3-[(oxolan-3-yl)amino]pyridazine-4-carboxylate

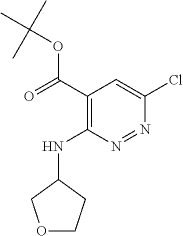 [SMILES: CC(C)(C)OC(=O)c1cc(Cl)nnc1NC1CCOC1]

Intermediate 232 was prepared following the procedure used for the synthesis of Intermediate 94 starting from tert-butyl 3,6-dichloropyridazine-4-carboxylate (Intermediate 212, 500 mg, 2.01 mmol) and 3-aminotetrahydrofuran (184 mg, 2.11 mmol) to afford title compound (356 mg, 1.19 mmol, 59% yield).

LC-MS (ESI): m/z (M+1): 300.2 (Method 1)

### Intermediate 233: tert-butyl 6-chloro-3-[methyl(oxolan-3-yl)amino]pyridazine-4-carboxylate

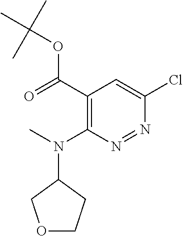 [SMILES: CN(c1nnc(Cl)cc1C(=O)OC(C)(C)C)C1CCOC1]

Intermediate 233 was prepared following the procedure used for the synthesis of Intermediate 223 starting from tert-butyl 6-chloro-3-[(oxolan-3-yl)amino]pyridazine-4-carboxylate (Intermediate 232, 356 mg, 1.19 mmol) to afford title compound (220 mg, 0.70 mmol, 59% yield). LC-MS (ESI): m/z (M+1): 314.1 (Method 1)

### Intermediate 234: tert-butyl 6-(5-chloro-2-fluorophenyl)-3-[methyl(oxolan-3-yl)amino]pyridazine-4-carboxylate

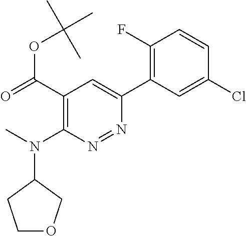 [SMILES: CN(c1nnc(-c2cc(Cl)ccc2F)cc1C(=O)OC(C)(C)C)C1CCOC1]

Intermediate 234 was prepared following the procedure used for the synthesis of Intermediate 8, starting from tert-butyl 6-chloro-3-[methyl(oxolan-3-yl)amino]pyridazine-4-carboxylate (Intermediate 233, 220 mg, 0.70 mmol) and 5-chloro-2-fluorobenzeneboronic acid (245 mg, 1.41 mmol) in presence of Pd(dppf)Cl₂ (103 mg, 0.14 mmol) to afford title compound (230 mg, 0.56 mmol, 80% yield).

LC-MS (ESI): m/z (M+1): 408.3 (Method 1)

### Intermediate 235: 6-(5-chloro-2-fluorophenyl)-3-[methyl(oxolan-3-yl)amino]pyridazine-4-carboxylic acid trifluoroacetic acid salt

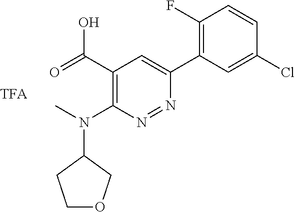 [SMILES: CN(c1nnc(-c2cc(Cl)ccc2F)cc1C(=O)O)C1CCOC1]

Intermediate 235 was prepared following the procedure used for the synthesis of Intermediate 215, starting from tert-butyl 6-(5-chloro-2-fluorophenyl)-3-[methyl(oxolan-3-yl)amino]pyridazine-4-carboxylate (Intermediate 234, 230 mg, 0.56 mmol) to afford title compound (260 mg, 0.56 mmol, 99% yield) as trifluoroacetic salt.

LC-MS (ESI): m/z (M+1): 352.2 (Method 1)

### Intermediate 236: 6-(5-chloro-2-fluorophenyl)-N3-methyl-N3-(oxolan-3-yl)pyridazine-3,4-diamine

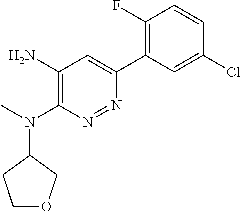 [SMILES: CN(c1nnc(-c2cc(Cl)ccc2F)cc1N)C1CCOC1]

Intermediate 236 was prepared following the procedure used for the synthesis of Intermediate 231, starting from 6-(5-chloro-2-fluorophenyl)-3-[methyl(oxolan-3-yl)amino]pyridazine-4-carboxylic acid trifluoroacetic acid salt (Intermediate 235, 260 mg, 0.56 mmol) to afford title compound (76 mg, 0.23 mmol, 42% yield).

LC-MS (ESI): m/z (M+1): 323.2 (Method 1)

### Intermediate 237: 3-[(methylamino)methyl]oxolan-2-one

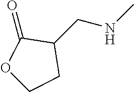

2 M methylamine in THF (3.82 mL, 7.65 mmol) was added to a solution of α-methylene-γ-butyrolactone (0.22 mL, 2.55 mmol) in THE (1 mL). The mixture was stirred at RT overnight, then volatiles were removed under vacuum to afford title compound (350 mg, recovery assumed quantitative) used as such in the next step.

LC-MS (ESI): m/z (M+1): 129.9 (Method 1)

### Intermediate 238: tert-butyl 6-chloro-3-{methyl[(2-oxooxolan-3-yl)methyl]amino}pyridazine-4-carboxylate

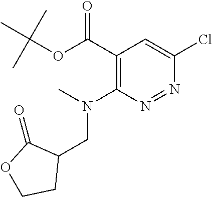 [SMILES: CN(CC1CCOC1=O)c1nnc(Cl)cc1C(=O)OC(C)(C)C]

Intermediate 238 was prepared following the procedure used for the synthesis of Intermediate 94 starting from tert-butyl 3,6-dichloropyridazine-4-carboxylate (Intermediate 212, 200 mg, 0.80 mmol) and 3-[(methylamino)methyl]oxolan-2-one (Intermediate 237, 207 mg, 1.71 mmol) to afford title compound (170 mg, 0.50 mmol, 62% yield). LC-MS (ESI): m/z (M+1): 342.2 (Method 1)

### Intermediate 239: tert-butyl 6-(5-chloro-2-fluorophenyl)-3-{methyl[(2-oxooxolan-3-yl)methyl]amino}pyridazine-4-carboxylate

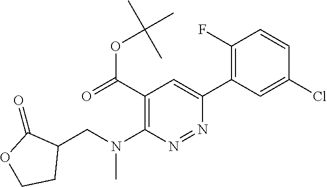 [SMILES: CN(CC1CCOC1=O)c1nnc(-c2cc(Cl)ccc2F)cc1C(=O)OC(C)(C)C]

Intermediate 239 was prepared following the procedure used for the synthesis of Intermediate 8, starting from Intermediate 238 (170 mg, 0.49 mmol) and 5-chloro-2-fluorobenzeneboronic acid (170 mg, 0.98 mmol) in presence of Pd(dppf)Cl₂ (71 mg, 0.10 mmol) to afford title compound (120 mg, 0.27 mmol, 56% yield).

LC-MS (ESI): m/z (M+1): 436.3 (Method 1)

### Intermediate 240: 6-(5-chloro-2-fluorophenyl)-3-{methyl[(2-oxooxolan-3-yl)methyl]amino}pyridazine-4-carboxylic acid trifluoroacetic acid salt

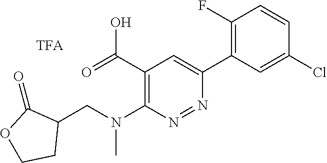 [SMILES: CN(CC1CCOC1=O)c1nnc(-c2cc(Cl)ccc2F)cc1C(=O)O]

Intermediate 240 was prepared following the procedure used for the synthesis of Intermediate 215, starting from Intermediate 239 (120 mg, 0.27 mmol) to afford title compound (134 mg, 0.27 mmol, 99% yield) as trifluoroacetic salt.

LC-MS (ESI): m/z (M+1): 380.2 (Method 1)

### Intermediate 241: 3-({[4-amino-6-(5-chloro-2-fluorophenyl)pyridazin-3-yl](methyl)amino}methyl)oxolan-2-one

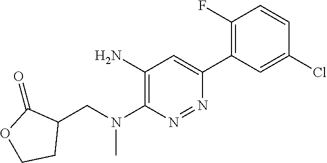 [SMILES: CN(CC1CCOC1=O)c1nnc(-c2cc(Cl)ccc2F)cc1N]

Intermediate 241 was prepared following the procedure used for the synthesis of Intermediate 231, starting from Intermediate 240 (134 mg, 0.27 mmol) to afford title compound (60 mg, 0.17 mmol, 63% yield). LC-MS (ESI): m/z (M+1): 351.3 (Method 1)

### Intermediate 242: tert-butyl N-(4,4,4-trifluoro-3-hydroxybutyl)carbamate

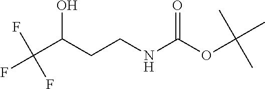 [SMILES: CC(C)(C)OC(=O)NCCC(O)C(F)(F)F]

4-amino-1,1,1-trifluoro-butan-2-ol (360 mg, 2.52 mmol) was dissolved in DCM (5 mL). TEA (0.39 mL, 2.77 mmol) and di-tert-butyl dicarbonate (604 mg, 2.77 mmol) were subsequently added, and the reaction was stirred at RT for 4 hrs. The mixture was washed with saturated NH₄Cl solution, the organic phase was dried and evaporated to afford title compound (620 mg, 2.52 mmol, quantitative yield).

¹H NMR (400 MHz, DMSO-d₆) δ ppm 6.85 (br. s., 1H), 6.11 (d, J=6.60 Hz, 1H), 3.87-4.02 (m, 1H), 2.97-3.14 (m, 2H), 1.65-1.75 (m, 1H), 1.49-1.60 (m, 1H), 1.37 (s, 9H).

### Intermediate 243: 1,1,1-trifluoro-4-(methylamino)butan-2-ol

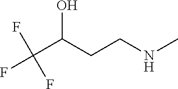

2 M lithium aluminum hydride in THE (2.55 mL, 5.1 mmol) was added dropwise to a solution of Intermediate 242 (620 mg, 2.52 mmol) in THE (12 mL). The resulting solution was refluxed for 1 h, then the mixture was cooled with an ice bath and Na₂SO₄·10 H₂O was added portion wise until gas evolution ceased. The mixture was diluted with EtOAc and filtered over a Celite® pad. Volatiles were removed under vacuum to afford title compound (340 mg, 2.16 mmol, 85% yield), used as such in the next step.

LC-MS (ESI): m/z (M+1): 158.0 (Method 2)

### Intermediate 244: tert-butyl 6-chloro-3-[methyl(4,4,4-trifluoro-3-hydroxybutyl)amino]pyridazine-4-carboxylate

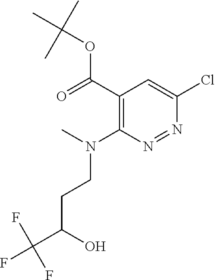 [SMILES: CN(CCC(O)C(F)(F)F)c1nnc(Cl)cc1C(=O)OC(C)(C)C]

Intermediate 244 was prepared following the procedure used for the synthesis of Intermediate 94 starting from tert-butyl 3,6-dichloropyridazine-4-carboxylate (Intermediate 212, 490 mg, 1.97 mmol) and 1,1,1-trifluoro-4-(methylamino)butan-2-ol (Intermediate 243, 340 mg, 2.16 mmol) to afford title compound (336 mg, 0.91 mmol, 46% yield).

LC-MS (ESI): m/z (M+1): 370.3 (Method 1)

### Intermediate 245: tert-butyl 6-(5-chloro-2-fluorophenyl)-3-[methyl(4,4,4-trifluoro-3-hydroxybutyl)amino]pyridazine-4-carboxylate

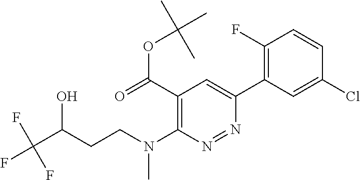 [SMILES: CN(CCC(O)C(F)(F)F)c1nnc(-c2cc(Cl)ccc2F)cc1C(=O)OC(C)(C)C]

Pd(PPh₃)₄(157 mg, 0.14 mmol) was added to a degassed mixture of tert-butyl 6-chloro-3-[methyl(4,4,4-trifluoro-3-hydroxybutyl)amino]pyridazine-4-carboxylate (Intermediate 244, 335 mg, 0.91 mmol) and 5-chloro-2-fluorobenzeneboronic acid (632 mg, 3.62 mmol) in a mixture of 2 M Na₂CO₃ (4.79 mL, 9.58 mmol), toluene (14 mL), and ethanol (9 mL). The mixture was heated at 105° C. for 90 min. The mixture was cooled to RT, diluted with EtOAc, the organic phase was separated, dried and evaporated. The crude material was purified by flash chromatography on Biotage silica cartridge (from c-Hex to 20% EtOAc) to afford title compound (300 mg, 0.65 mmol, 70% yield).

LC-MS (ESI): m/z (M+1): 464.3 (Method 1)

### Intermediate 246: 6-(5-chloro-2-fluorophenyl)-3-[methyl(4,4,4-trifluoro-3-hydroxybutyl)amino]pyridazine-4-carboxylic acid trifluoroacetic acid salt

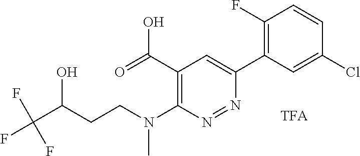 [SMILES: CN(CCC(O)C(F)(F)F)c1nnc(-c2cc(Cl)ccc2F)cc1C(=O)O]

Intermediate 246 was prepared following the procedure used for the synthesis of Intermediate 215, starting from tert-butyl 6-(5-chloro-2-fluorophenyl)-3-[methyl(4,4,4-trifluoro-3-hydroxybutyl)amino]pyridazine-4-carboxylate (Intermediate 245, 265 mg, 0.57 mmol) to afford title compound (300 mg, 0.57 mmol, quantitative yield) as trifluoroacetic salt.

LC-MS (ESI): m/z (M+1): 408.2 (Method 1)

### Intermediate 247: 4-{[4-amino-6-(5-chloro-2-fluorophenyl)pyridazin-3-yl](methyl)amino}-1,1,1-trifluorobutan-2-ol

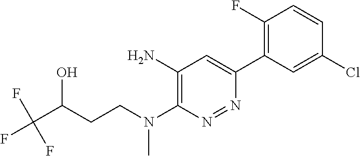 [SMILES: CN(CCC(O)C(F)(F)F)c1nnc(-c2cc(Cl)ccc2F)cc1N]

Intermediate 247 was prepared following the procedure used for the synthesis of Intermediate 231, starting from 6-(5-chloro-2-fluorophenyl)-3-[methyl(4,4,4-trifluoro-3-hydroxybutyl)amino]pyridazine-4-carboxylic acid trifluoroacetic acid salt (Intermediate 246, 300 mg, 0.57 mmol) to afford title compound (98 mg, 0.29 mmol, 50% yield).

LC-MS (ESI): m/z (M+1): 379.2 (Method 1)

### Intermediate 248: ethyl 2,2-dimethyl-2H-1,3-benzodioxole-5-carboxylate

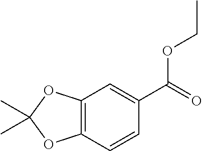 [SMILES: CCOC(=O)c1ccc2c(c1)OC(C)(C)O2]

To a stirred solution of ethyl 3,4-dihydroxybenzoate (1.5 g, 8.23 mmol) in acetone (4.23 mL, 57.64 mmol) and toluene (5 mL), at RT, phosphorus trichloride (0.58 mL, 6.59 mmol) was added dropwise, and the resulting reaction mixture was stirred at RT for 20 hrs. EtOAc (12 mL) was added followed by a saturated NaHCO₃ aqueous solution and the mixture was stirred for 15 min then extracted with EtOAc. The organic phase was separated, washed with brine, dried over Na₂SO₄ and the solvent removed under reduced pressure. The crude material was purified by flash chromatography on Biotage silica cartridge (from c-Hex to 25% EtOAc) to afford title compound (1.04 g, 4.7 mmol, 57% yield). LC-MS (ESI): m/z (M+1): 223.1 (Method 1)

### Intermediate 249: (2,2-dimethyl-2H-1,3-benzodioxol-5-yl)methanol

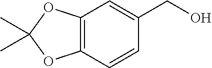

Intermediate 249 was prepared following the procedure used for the synthesis of Intermediate 154 starting from ethyl 2,2-dimethyl-2H-1,3-benzodioxole-5-carboxylate (Intermediate 248, 1.04 g, 4.7 mmol) to afford title compound (574 mg, 3.18 mmol, 67% yield).

¹H NMR (400 MHz, DMSO-d₆) δ ppm 6.65-6.83 (m, 3H) 5.03 (t, J=5.83 Hz, 1H) 4.36 (d, J=5.72 Hz, 2H) 1.55-1.71 (m, 6H).

### Intermediate 250: 6-chloro-3-[(2,2-dimethyl-2H-1,3-benzodioxol-5-yl)methoxy]pyridazin-4-amine

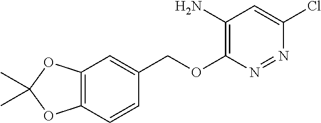 [SMILES: CC1(C)Oc2ccc(COc3nnc(Cl)cc3N)cc2O1]

Intermediate 250 was prepared following the procedure used for the synthesis of Intermediate 10 starting from 3,6-dichloropyridazin-4-amine (173 mg, 1.05 mmol) and (2,2-dimethyl-2H-1,3-benzodioxol-5-yl)methanol (Intermediate 249, 570 mg, 3.16 mmol) to afford title compound (180 mg, 0.58 mmol, 55% yield).

LC-MS (ESI): m/z (M+1): 308.1 (Method 2)

### Intermediate 251: 6-(5-chloro-2-fluorophenyl)-3-[(2,2-dimethyl-2H-1,3-benzodioxol-5-yl)methoxy]pyridazin-4-amine

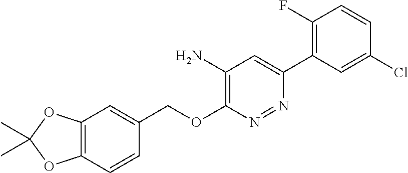 [SMILES: CC1(C)Oc2ccc(COc3nnc(-c4cc(Cl)ccc4F)cc3N)cc2O1]

Intermediate 251was prepared following the procedure used for the synthesis of Intermediate 8, starting from 6-chloro-3-[(2,2-dimethyl-2H-1,3-benzodioxol-5-yl)methoxy]pyridazin-4-amine (Intermediate 250, 180 mg, 0.58 mmol) and 5-chloro-2-fluorobenzeneboronic acid (163 mg, 1.60 mmol) in presence of Pd(dppf)Cl₂ (86 mg, 0.12 mmol) to afford title compound (121 mg, 0.30 mmol, 51% yield).

LC-MS (ESI): m/z (M+1): 402.1 (Method 2)

### Intermediate 252: Cis 3-(hydroxymethyl)-1-methylcyclobutan-1-ol

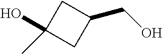

To a stirred solution of cis-3-hydroxy-3-methylcyclobutanecarboxylic acid (1.2 g, 9.22 mmol) in THE (18 mL), at 0° C. and under a N₂, borane tetrahydrofuran complex 1 M in THF (18.44 mL, 18.44 mmol) was added dropwise. After 5 min the ice-bath was removed, and the resulting reaction mixture was stirred at RT for 2.5 hrs. The mixture was cooled to 0° C. and quenched by adding MeOH. After 5 min the ice-bath was removed, and it was stirred at RT for 30 min. The mixture was then concentrated under reduced pressure and the crude material was purified by flash chromatography on Biotage silica cartridge (from cHex to 80% EtOAc) to afford title compound (1.1 g, 9.47 mmol, quantitative yield).

### Intermediate 253: Cis 3-{[(6-chloro-4-{[(2,4-dimethoxyphenyl)methyl]amino}pyridazin-3-yl)oxy]methyl}-1-methylcyclobutan-1-ol

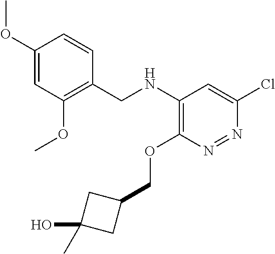 [SMILES: COc1ccc(CNc2cc(Cl)nnc2OC[C@H]2C[C@@](C)(O)C2)c(OC)c1]

Intermediate 253 was prepared following the procedure used for the synthesis of Intermediate 7 starting from 3,6-dichloro-N-[(2,4-dimethoxyphenyl)methyl]pyridazin-4-amine (Intermediate 6, 2.06 g, 6.56 mmol), and cis 3-(hydroxymethyl)-1-methylcyclobutan-1-ol (Intermediate 252, 1 g, 8.6 mmol) at 115° C. to afford title compound (938 mg, 2.38 mmol, 36% yield).

LC-MS (ESI): m/z (M+1): 394.3 (Method 1)

### Intermediate 254: Cis 3-({[6-(5-chloro-2-fluorophenyl)-4-{[(2,4-dimethoxyphenyl)methyl]amino}pyridazin-3-yl]oxy}methyl)-1-methylcyclobutan-1-ol

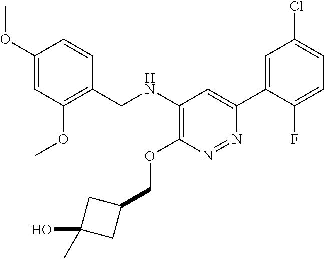 [SMILES: COc1ccc(CNc2cc(-c3cc(Cl)ccc3F)nnc2OC[C@H]2C[C@@](C)(O)C2)c(OC)c1]

Intermediate 254 was prepared following the procedure used for the synthesis of Intermediate 245 starting from cis 3-{[(6-chloro-4-{[(2,4-dimethoxyphenyl)methyl]amino}pyridazin-3-yl)oxy]methyl}-1-methylcyclobutan-1-ol (Intermediate 253, 380 mg, 0.96 mmol) and 5-chloro-2-fluorobenzeneboronic acid (674 mg, 3.86 mmol) to afford title compound (406 mg, 0.83 mmol, 86% yield).

LC-MS (ESI): m/z (M+1): 488.3 (Method 1)

### Intermediate 255: Cis 3-({[4-amino-6-(5-chloro-2-fluorophenyl)pyridazin-3-yl]oxy}methyl)-1-methylcyclobutan-1-ol

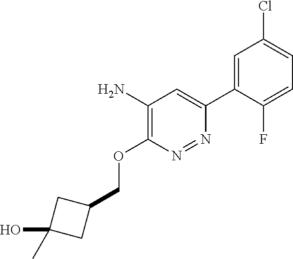 [SMILES: C[C@]1(O)C[C@@H](COc2nnc(-c3cc(Cl)ccc3F)cc2N)C1]

Intermediate 255 was prepared following the procedure used for the synthesis of Intermediate 9 starting from Intermediate 254 (838 mg, 1.72 mmol) to afford title compound (148 mg, 0.44 mmol, 25% yield). LC-MS (ESI): m/z (M+1): 338.1 (Method 1)

### Intermediate 256: methyl 3-(hydroxymethyl)bicyclo[1.1.1]pentane-1-carboxylate

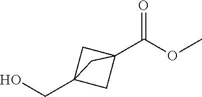

To an ice-cooled solution of 3-(methoxycarbonyl)bicyclo[1.1.1]pentane-1-carboxylic acid (3 g, 17.63 mmol) in THE (35.2 mL), borane tetrahydrofuran complex 1 M in THE (17.63 mL, 17.63 mmol) was added and the mixture was slowly allowed to reach RT and stirred for 16 hrs. The mixture was cooled to 0° C. and water was added dropwise followed by solid K₂CO₃ (˜2 eq) then extracted with EtOAc (3×). The combined organic layers were dried and evaporated under vacuum. The crude material was purified by flash chromatography on Biotage silica cartridge (from cHex to 60% EtOAc) to afford title compound (2.1 g, 13.45 mmol, 76% yield).

LC-MS (ESI): m/z (M+1): 157.1 (Method 1)

### Intermediate 257: methyl 3-{[(6-chloro-4-{[(2,4-dimethoxyphenyl)methyl]amino}pyridazin-3-yl)oxy]methyl}bicyclo[1.1.1]pentane-1-carboxylate

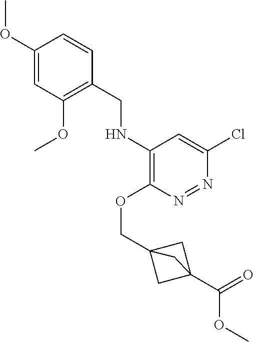 [SMILES: COC(=O)C12CC(COc3nnc(Cl)cc3NCc3ccc(OC)cc3OC)(C1)C2]

Intermediate 257 was prepared following the procedure used for the synthesis of Intermediate 7 starting from Intermediate 6 (658 mg, 2.09 mmol), and methyl 3-(hydroxymethyl)bicyclo[1.1.1]pentane-1-carboxylate (Intermediate 256, 463 mg, 2.93 mmol) at 95° C. to afford title compound (660 mg, 1.52 mmol, 73% yield). LC-MS (ESI): m/z (M+1): 434.4 (Method 1)

### Intermediate 258: methyl 3-({[6-(5-chloro-2-fluorophenyl)-4-{[(2,4-dimethoxyphenyl)methyl]amino}pyridazin-3-yl]oxy}methyl)bicyclo[1.1.1]pentane-1-carboxylate

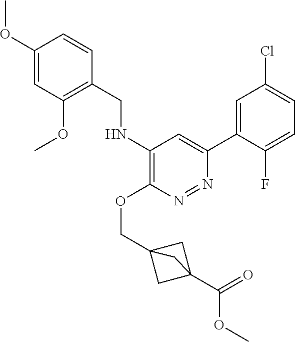 [SMILES: COC(=O)C12CC(COc3nnc(-c4cc(Cl)ccc4F)cc3NCc3ccc(OC)cc3OC)(C1)C2]

Intermediate 258 was prepared following the procedure used for the synthesis of Intermediate 8 starting from Intermediate 257 (70 mg, 0.16 mmol) and 5-chloro-2-fluorobenzeneboronic acid (42.2 mg, 0.24 mmol) in presence of Pd(dppf)Cl₂ (23.6 mg, 0.03 mmol) to afford title compound (49 mg, 0.09 mmol, 58% yield).

LC-MS (ESI): m/z (M+1): 528.3 (Method 1)

### Intermediate 259: methyl 3-({[4-amino-6-(5-chloro-2-fluorophenyl)pyridazin-3-yl]oxy}methyl)bicyclo[1.1.1]pentane-1-carboxylate

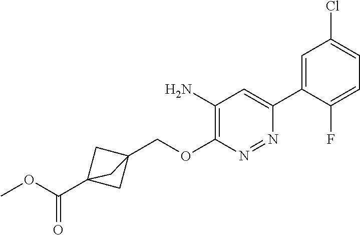 [SMILES: COC(=O)C12CC(COc3nnc(-c4cc(Cl)ccc4F)cc3N)(C1)C2]

Intermediate 259 was prepared following the procedure used for the synthesis of Intermediate 64, starting from Intermediate 258 (330 mg, 0.63 mmol) to afford title compound (164 mg, 0.43 mmol, 69% yield).

LC-MS (ESI): m/z (M+1): 378.3 (Method 2)

### Intermediate 260: 3-{[benzyl(methyl)amino]methyl}oxolan-2-one

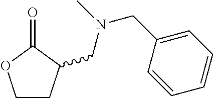 [SMILES: CN(Cc1ccccc1)CC1CCOC1=O]

N-methyl-1-phenylmethanamine (0.99 mL, 7.65 mmol), was added to a solution of 3-methylene-2-oxolanone (300 mg, 3.06 mmol) in THE (4 mL), the vial was sealed and stirred at RT overnight. The reaction mixture was concentrated under reduce pressure and the crude material was purified by flash chromatography on Biotage silica cartridge (from cHex to 40% EtOAc) to afford title compound (552 mg, 2.52 mmol, 82% yield).

LC-MS (ESI): m/z (M+1): 220.2 (Method 2)

### Intermediate 261: 3-{[benzyl(methyl)amino]methyl}-3-methyloxolan-2-one

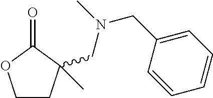 [SMILES: CN(Cc1ccccc1)CC1(C)CCOC1=O]

To a stirred solution of Intermediate 260 (548 mg, 2.5 mmol) in THF (12 mL), at -78° C. and under N₂, a solution of lithium bis(trimethylsilyl)amide 1M in THE (3 mL, 3 mmol) was added dropwise. The reaction mixture was stirred at −78° C. for 50 min, then iodomethane (0.35 mL, 5.62 mmol) was added dropwise. The resulting reaction mixture was stirred for 20 min at −78° C. then was slowly allowed to reach RT and stirred overnight at RT. The reaction mixture was diluted with EtOAc and a aqueous concentrated solution of NaHCO₃ was added. The mixture was extracted with additional EtOAc, the organic phase was washed with water, dried and evaporated. The crude material was purified by flash chromatography on Biotage silica cartridge (from cHex to 25% EtOAc) to afford title compound (406 mg, 1.74 mmol, 70% yield). LC-MS (ESI): m/z (M+1): 234.9 (Method 2)

### Intermediate 262: 3-methyl-3-[(methylamino)methyl]oxolan-2-one

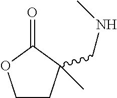

To a solution of 3-{[benzyl(methyl)amino]methyl}-3-methyloxolan-2-one (Intermediate 261, 406 mg, 1.74 mmol) in MeOH (65 mL), at RT, 10% Pd/C 55-65% wet (200 mg, 1.13 mmol) was added and the resulting mixture was hydrogenated at atmospheric pressure. The mixture was filtered over celite, and the filtrate was concentrated under reduced pressure to afford title compound (220 mg, 1.54 mmol, 88% yield) used as such. LC-MS (ESI): m/z (M+1): 144.0 (Method 2)

### Intermediate 263: tert-butyl 6-chloro-3-{methyl[(3-methyl-2-oxooxolan-3-yl)methyl]amino}pyridazine-4-carboxylate

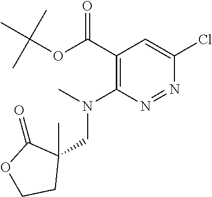 [SMILES: CN(C[C@]1(C)CCOC1=O)c1nnc(Cl)cc1C(=O)OC(C)(C)C]

Intermediate 263 was prepared following the procedure used for the synthesis of Intermediate 94 starting from tert-butyl 3,6-dichloropyridazine-4-carboxylate (Intermediate 212, 220 mg, 0.88 mmol) and 3-methyl-3-[(methylamino)methyl]oxolan-2-one (Intermediate 262, 220 mg, 1.74 mmol) to afford title compound (266 mg, 0.75 mmol, 85% yield).

LC-MS (ESI): m/z (M+1): 356.2 (Method 1)

### Intermediate 264: tert-butyl 6-(5-chloro-2-fluorophenyl)-3-{methyl[(3-methyl-2-oxooxolan-3-yl)methyl]amino}pyridazine-4-carboxylate

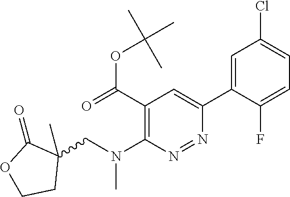 [SMILES: CN(CC1(C)CCOC1=O)c1nnc(-c2cc(Cl)ccc2F)cc1C(=O)OC(C)(C)C]

Intermediate 264 was prepared following the procedure used for the synthesis of Intermediate 8, starting from tert-butyl 6-chloro-3-{methyl[(3-methyl-2-oxooxolan-3-yl)methyl]amino}pyridazine-4-carboxylate (Intermediate 263, 266 mg, 0.75 mmol) and 5-chloro-2-fluorobenzeneboronic acid (261 mg, 1.50 mmol) in presence of Pd(dppf)Cl₂ (110 mg, 0.15 mmol) to afford title compound (265 mg, 0.59 mmol, 79% yield). LC-MS (ESI): m/z (M+1): 450.3 (Method 1)

### Intermediate 265: 6-(5-chloro-2-fluorophenyl)-3-{methyl[(3-methyl-2-oxooxolan-3-yl)methyl]amino}pyridazine-4-carboxylic acid trifluoroacetic acid salt

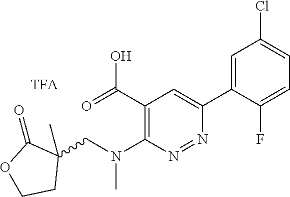 [SMILES: CN(CC1(C)CCOC1=O)c1nnc(-c2cc(Cl)ccc2F)cc1C(=O)O]

Intermediate 265 was prepared following the procedure used for the synthesis of Intermediate 215, starting from Intermediate 264 (265 mg, 0.59 mmol) to afford title compound (302 mg, 0.29 mmol, quantitative yield). LC-MS (ESI): m/z (M+1): 394.2 (Method 1).

### Intermediate 266: 3-({[4-amino-6-(5-chloro-2-fluorophenyl)pyridazin-3-yl](methyl)amino}methyl)-3-methyloxolan-2-one

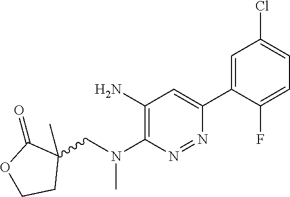 [SMILES: CN(CC1(C)CCOC1=O)c1nnc(-c2cc(Cl)ccc2F)cc1N]

Intermediate 266 was prepared following the procedure used for the synthesis of Intermediate 231, starting from 6-(5-chloro-2-fluorophenyl)-3-{methyl[(3-methyl-2-oxooxolan-3-yl)methyl]amino}pyridazine-4-carboxylic acid trifluoroacetic acid salt (Intermediate 265, 299 mg, 0.59 mmol) to afford title compound (153 mg, 0.42 mmol, 71% yield). LC-MS (ESI): m/z (M+1): 365.2 (Method 1)

### Intermediate 267: methyl 4-{4-[(tert-butoxy)carbonyl]-6-chloropyridazin-3-yl}morpholine-2-carboxylate

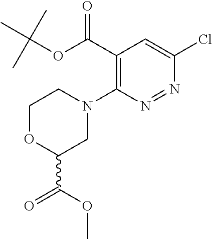 [SMILES: COC(=O)C1CN(c2nnc(Cl)cc2C(=O)OC(C)(C)C)CCO1]

Intermediate 267 was prepared following the procedure used for the synthesis of Intermediate 94 starting from tert-butyl 3,6-dichloropyridazine-4-carboxylate (Intermediate 212, 500 mg, 2.01 mmol) and methyl morpholine-2-carboxylate hydrochloride (365 mg, 2.01 mmol) to afford title compound (375 mg, 1.05 mmol, 52% yield). LC-MS (ESI): m/z (M+1): 358.1 (Method 1)

### Intermediate 268: methyl 4-{4-[(tert-butoxy)carbonyl]-6-(5-chloro-2-fluorophenyl)pyridazin-3-yl}morpholine-2-carboxylate

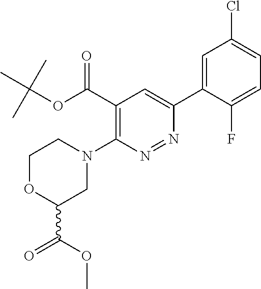 [SMILES: COC(=O)C1CN(c2nnc(-c3cc(Cl)ccc3F)cc2C(=O)OC(C)(C)C)CCO1]

Intermediate 268 was prepared following the procedure used for the synthesis of Intermediate 8, starting from Intermediate 267 (375 mg, 1.05 mmol) and 5-chloro-2-fluorobenzeneboronic acid (366 mg, 2.10 mmol) in presence of Pd(dppf)Cl₂ (154 mg, 0.21 mmol) to afford title compound (260 mg, 0.57 mmol, 55% yield). LC-MS (ESI): m/z (M+1): 452.2 (Method 1)

### Intermediate 269: 6-(5-chloro-2-fluorophenyl)-3-[2-(methoxycarbonyl)morpholin-4-yl]pyridazine-4-carboxylic acid trifluoroacetic acid salt

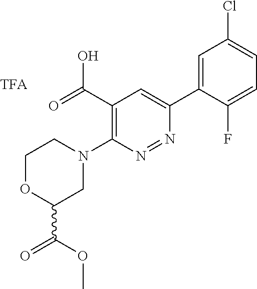 [SMILES: COC(=O)C1CN(c2nnc(-c3cc(Cl)ccc3F)cc2C(=O)O)CCO1]

Intermediate 269 was prepared following the procedure used for the synthesis of Intermediate 215, starting from Intermediate 268 (245 mg, 0.54 mmol) to afford title compound (0.54 mmol, quantitative yield).

LC-MS (ESI): m/z (M+1): 396.2 (Method 1).

### Intermediate 270: methyl 4-[4-amino-6-(5-chloro-2-fluorophenyl)pyridazin-3-yl]morpholine-2-carboxylate

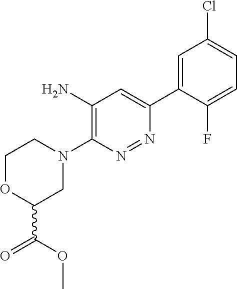 [SMILES: COC(=O)C1CN(c2nnc(-c3cc(Cl)ccc3F)cc2N)CCO1]

Intermediate 270 was prepared following the procedure used for the synthesis of Intermediate 231, starting from Intermediate 269 (0.54 mmol) to afford title compound (150 mg, 0.41 mmol, 76% yield). LC-MS (ESI): m/z (M+1): 367.1 (Method 1)

### Intermediate 271: 3-(4-methylpiperazin-1-yl)propanamide

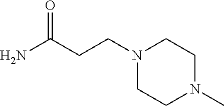

1-methylpiperazine (1.56 mL, 14.07 mmol) and 2-propenamide (1.0 g, 14.07 mmol) were mixed in H₂O (12 ml) and stirred at 60° C. for 6 hrs. H₂O was removed under vacuum to afford title compound (2.4 g, 14.02 mmol, 99% yield).

### Intermediate 272: N-(6-chloropyrimidin-4-yl)-3-(4-methylpiperazin-1-yl)propanamide

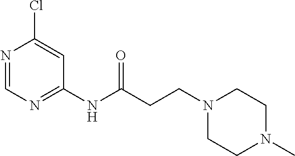 [SMILES: CN1CCN(CCC(=O)Nc2cc(Cl)ncn2)CC1]

Cs₂CO₃ (3.61 g, 11 mmol), Xantphos (382 mg, 0.66 mmol), 4,6-dichloropyrimidine (820 mg, 5.5 mmol), and 3-(4-methylpiperazin-1-yl)propanamide (Intermediate 271, 942 mg, 5.5 mmol) were mixed in 1,2-dimethoxyethane (39 mL), N₂ was bubbled for 5 min then Pd(OAc)₂ (62 mg, 0.28 mmol) was added. The mixture was heated at 75° C. for 1.5 hrs. The mixture was cooled to RT, filtered over celite using EtOAC and volatiles were removed under reduced pressure. The crude material was purified by flash chromatography on Biotage NH cartridge (from cHex to 100% EtOAc) to afford title compound (659 mg, 2.32 mmol, 42% yield). LC-MS (ESI): m/z (M+1): 284.1 (Method 2)

### Intermediate 273: ethyl 2,2-dimethyl-3-(2,2,2-trifluoroacetamido)propanoate

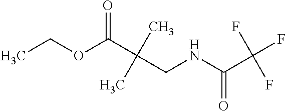 [SMILES: CCOC(=O)C(C)(C)CNC(=O)C(F)(F)F]

Trifluoroacetic anhydride (0.92 mL, 6.61 mmol) was added dropwise to a solution of DIPEA (2.4 mL, 13.76 mmol) and ethyl 3-amino-2,2-dimethylpropanoate hydrochloride (1 g, 5.5 mmol) in DCM (20 mL) at RT. The resulting reaction solution was stirred at the same temperature for 3 hrs. The mixture was treated with aqueous 1 N HCl and the product was extracted in DCM. Phases were separated and the organic one was dried over Na₂SO₄, filtered and evaporated to afford title compound (5.5 mmol, quantitative yield) that was used directly in the following step.

### Intermediate 274: ethyl 2,2-dimethyl-3-(2,2,2-trifluoro-N-methylacetamido)propanoate

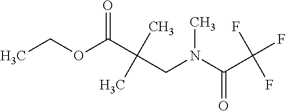 [SMILES: CCOC(=O)C(C)(C)CN(C)C(=O)C(F)(F)F]

To a solution of Intermediate 273 (5.5 mmol) in THE (22 mL) at 0° C. iodomethane (0.6 mL, 9.7 mmol) was added followed by portion-wise addition of NaH 60% dispersion in oil (597 mg, 14.92 mmol) over 10 min. The mixture was stirred overnight at RT. The mixture was cooled with an ice bath and aqueous 1 N HCl was added, followed by Et₂O. The aqueous phase was extracted twice with Et₂O, the combined organic phases were dried over Na₂SO₄, filtered and evaporated. The crude material was purified by flash chromatography on Biotage silica cartridge (from cHex to 10% EtOAc) to afford title compound (1.31 g, 5.13 mmol, 93% yield).

### Intermediate 275: ethyl 2,2-dimethyl-3-(methylamino)propanoate

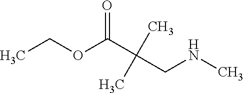

Ethyl 2,2-dimethyl-3-(2,2,2-trifluoro-N-methylacetamido)propanoate (Intermediate 274, 1.31 g, 5.13 mmol) was dissolved in 7 N NH₃ in MeOH (22 mL, 154 mmol) and stirred overnight at RT. Volatiles were removed under vacuum to afford title compound and its corresponding methyl ester as a 2:1 mixture (740 mg, 4.65 mmol, 91% yield) that was used without further purification.

### Intermediate 276: tert-butyl 6-chloro-3-[(3-ethoxy-2,2-dimethyl-3-oxopropyl)(methyl)amino]pyridazine-4-carboxylate

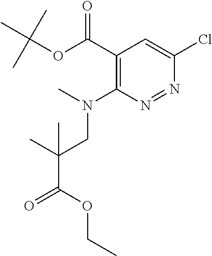 [SMILES: CCOC(=O)C(C)(C)CN(C)c1nnc(Cl)cc1C(=O)OC(C)(C)C]

Intermediate 276 was prepared following the procedure used for the synthesis of Intermediate 94 starting from Intermediate 212 (300 mg, 1.20 mmol) and ethyl 2,2-dimethyl-3-(methylamino)propanoate (Intermediate 275, 211 mg, 1.33 mmol) to afford title compound (120 mg, 0.32 mmol, 27% yield). LC-MS (ESI): m/z (M+1): 372.4 (Method 1)

### Intermediate 277: tert-butyl 6-(5-chloro-2-fluorophenyl)-3-[(3-ethoxy-2,2-dimethyl-3-oxopropyl)(methyl)amino]pyridazine-4-carboxylate

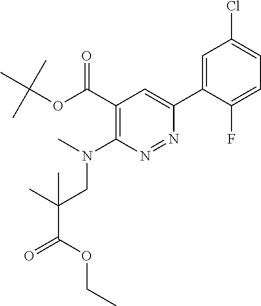 [SMILES: CCOC(=O)C(C)(C)CN(C)c1nnc(-c2cc(Cl)ccc2F)cc1C(=O)OC(C)(C)C]

Intermediate 277 was prepared following the procedure used for the synthesis of Intermediate 8, starting from Intermediate 276 (120 mg, 0.32 mmol) and 5-chloro-2-fluorobenzeneboronic acid (113 mg, 0.65 mmol) in presence of Pd(dppf)Cl₂ (47 mg, 0.06 mmol) to afford title compound (140 mg, 0.30 mmol, 93% yield).

LC-MS (ESI): m/z (M+1): 466.2 (Method 1)

### Intermediate 278: 6-(5-chloro-2-fluorophenyl)-3-[(3-ethoxy-2,2-dimethyl-3-oxopropyl)(methyl)amino]pyridazine-4-carboxylic acid trifluoroacetic salt

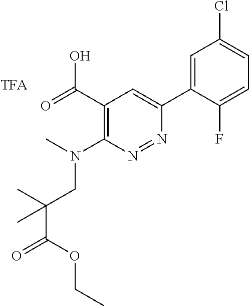 [SMILES: CCOC(=O)C(C)(C)CN(C)c1nnc(-c2cc(Cl)ccc2F)cc1C(=O)O]

Intermediate 278 was prepared following the procedure used for the synthesis of Intermediate 215, starting from Intermediate 277 (180 mg, 0.39 mmol) to afford title compound (0.39 mmol, quantitative yield). LC-MS (ESI): m/z (M+1): 410.3 (Method 1)

### Intermediate 279: ethyl 3-{[4-amino-6-(5-chloro-2-fluorophenyl)pyridazin-3-yl](methyl)amino}-2,2-dimethylpropanoate

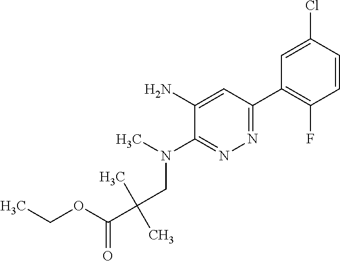 [SMILES: CCOC(=O)C(C)(C)CN(C)c1nnc(-c2cc(Cl)ccc2F)cc1N]

Intermediate 279 was prepared following the procedure used for the synthesis of Intermediate 231, starting from Intermediate 278 (0.39 mmol) to afford title compound (70 mg, 0.18 mmol, 46% yield). LC-MS (ESI): m/z (M+1): 381.4 (Method 1)

### Intermediate 280 (Enantiomer 1) and Intermediate 281 (Enantiomer 2): 4-{[4-amino-6-(5-chloro-2-fluorophenyl)pyridazin-3-yl](methyl)amino}-1,1,1-trifluorobutan-2-ol

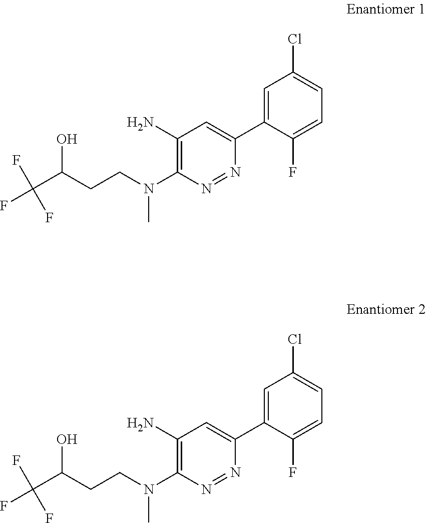 [SMILES: CN(CCC(O)C(F)(F)F)c1nnc(-c2cc(Cl)ccc2F)cc1N ; CN(CCC(O)C(F)(F)F)c1nnc(-c2cc(Cl)ccc2F)cc1N]

Racemate intermediate 247 (210 mg, 0.2 mmol) was separated into the single enantiomers by preparative chiral HPLC.

### Conditions:

| Column | Chiralpak AD-H (25 × 3.0 cm), 5μ | |
|---|---|---|
| Mobile phase | n-Hexane/(Ethanol + 0.1% isopropylamine) | |
| | 80/20% v/v | |
| Flow rate (ml/min) | 38 | ml/min |
| DAD detection | 220 | nm |
| Loop | 550 | μL |

Intermediate 280 was obtained as first eluted enantiomer (76 mg)
Rt.=10.9 min, ee 100%
LC-MS (ESI): m/z (M+1): 379.5 (Method 2)
Intermediate 281 was obtained as the second eluted enantiomer (32 mg).
Rt.=14.5 min, ee 98.6%
LC-MS (ESI): m/z (M+1): 379.5 (Method 2)

### Intermediate 282: tert-butyl 6-chloro-3-{2-[(propan-2-yloxy)carbonyl]azetidin-1-yl}pyridazine-4-carboxylate

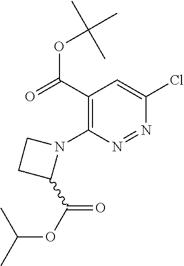 [SMILES: CC(C)OC(=O)C1CCN1c1nnc(Cl)cc1C(=O)OC(C)(C)C]

### Step 1

To a stirred mixture of azetidine-2-carboxylic acid (500 mg, 4.95 mmol) in propan-2-ol (10 mL, 130.8 mmol), at 0° C. and under N₂, thionyl dichloride (0.6 mL, 8.23 mmol) was added dropwise. After 5 min, the ice-bath was removed and the resulting reaction mixture was stirred at RT for 2.5 hrs, then heated at 60° C. for 1 h. The reaction mixture was concentrated under reduced pressure to afford propan-2-yl azetidine-2-carboxylate hydrochloride (4.95 mmol, quantitative yield) that was used as such in the next step.

### Step 2

Intermediate 282 was prepared following the procedure used for the synthesis of Intermediate 94 starting from Intermediate 212 (650 mg, 2.61 mmol) and propan-2-yl azetidine-2-carboxylate hydrochloride (from Step 1, 4.22 mmol) to afford title compound (764 mg, 2.15 mmol, 82% yield). LC-MS (ESI): m/z (M+1): 356.2 (Method 1)

### Intermediate 283: tert-butyl 6-(5-chloro-2-fluorophenyl)-3-{2-[(propan-2-yloxy)carbonyl]azetidin-1-yl}pyridazine-4-carboxylate

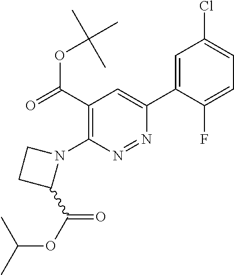 [SMILES: CC(C)OC(=O)C1CCN1c1nnc(-c2cc(Cl)ccc2F)cc1C(=O)OC(C)(C)C]

Intermediate 283 was prepared following the procedure used for the synthesis of Intermediate 8, starting from Intermediate 282 (764 mg, 2.15 mmol) and 5-chloro-2-fluorobenzeneboronic acid (562 mg, 3.22 mmol) in presence of Pd(dppf)Cl₂ (158 mg, 0.21 mmol) to afford title compound (935 mg, 2.08 mmol, 97% yield). LC-MS (ESI): m/z (M+1): 450.2 (Method 1)

### Intermediate 284: 6-(5-chloro-2-fluorophenyl)-3-{2-[(propan-2-yloxy)carbonyl]azetidin-1-yl}pyridazine-4-carboxylic acid trifluoroacetic salt

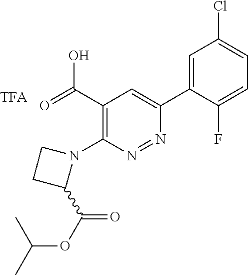 [SMILES: CC(C)OC(=O)C1CCN1c1nnc(-c2cc(Cl)ccc2F)cc1C(=O)O]

Intermediate 284 was prepared following the procedure used for the synthesis of Intermediate 215, starting from Intermediate 283 (935 mg, 2.08 mmol) to afford title compound (898 mg, 1.77 mmol, 85% yield). LC-MS (ESI): m/z (M+1): 394.1 (Method 1)

### Intermediate 285: propan-2-yl 1-[4-amino-6-(5-chloro-2-fluorophenyl)pyridazin-3-yl]azetidine-2-carboxylate

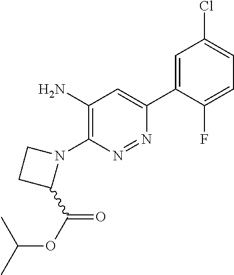 [SMILES: CC(C)OC(=O)C1CCN1c1nnc(-c2cc(Cl)ccc2F)cc1N]

Intermediate 285 was prepared following the procedure used for the synthesis of Intermediate 231, starting from Intermediate 284 (898 mg, 1.77 mmol) to afford title compound (360 mg, 0.99 mmol, 56% yield). LC-MS (ESI): m/z (M+1): 365.1 (Method 1)

### Intermediate 286: 3-{[benzyl(methyl)amino]methyl}-3-[(benzyloxy)methyl]oxolan-2-one

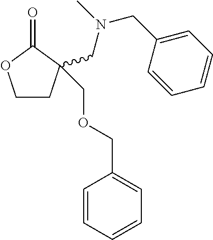 [SMILES: CN(Cc1ccccc1)CC1(COCc2ccccc2)CCOC1=O]

Intermediate 286 was prepared following the procedure used for the synthesis of Intermediate 261, starting from Intermediate 260 (3.69 g, 16.83 mmol) and benzyl chloromethyl ether (5.67 ml, 38.7 mmol) to afford title compound (2.71 g, 7.99 mmol, 47% yield). LC-MS (ESI): m/z (M+1): 340.2 (Method 4)

### Intermediate 287: tert-butyl 6-chloro-3-({[3-(hydroxymethyl)-2-oxooxolan-3-yl]methyl}(methyl)amino)pyridazine-4-carboxylate

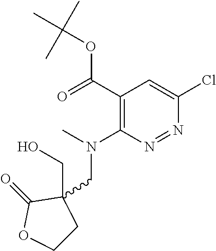 [SMILES: CN(CC1(CO)CCOC1=O)c1nnc(Cl)cc1C(=O)OC(C)(C)C]

### Step 1

To a stirred solution of Intermediate 286 (2.71 g, 7.99 mmol) in EtOAc (80 mL), at RT, 10% Pd over carbon 55-65% wet (1.36 g, 0.64 mmol) was added and the resulting mixture was hydrogenated at atmospheric pressure overnight.. The mixture was filtered over celite and concentrated under reduced pressure to afford 3-(hydroxymethyl)-3-[(methylamino)methyl]oxolan-2-one (7.99 mmol, quantitative yield) that was used as such in the next step.

### Step 2

Intermediate 287 was prepared following the procedure used for the synthesis of Intermediate 94 starting from Intermediate 212 (803 mg, 3.22 mmol) and 3-(hydroxymethyl)-3-[(methylamino)methyl]oxolan-2-one (from Step 1, 1.02 g, 6.45 mmol) to afford title compound (470 mg, 1.26 mmol, 36% yield).

LC-MS (ESI): m/z (M+1): 372.1 (Method 3)

### Intermediate 288: tert-butyl 6-(5-chloro-2-fluorophenyl)-3-({[3-(hydroxymethyl)-2-oxooxolan-3-yl]methyl}(methyl)amino)pyridazine-4-carboxylate

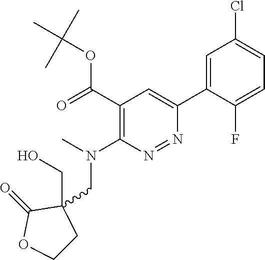 [SMILES: CN(CC1(CO)CCOC1=O)c1nnc(-c2cc(Cl)ccc2F)cc1C(=O)OC(C)(C)C]

Intermediate 288 was prepared following the procedure used for the synthesis of Intermediate 8, starting from Intermediate 287 (470 mg, 1.26 mmol) and 5-chloro-2-fluorobenzeneboronic acid (441 mg, 2.53 mmol) in presence of Pd(dppf)Cl₂ (186 mg, 0.26 mmol) to afford title compound (448 mg, 0.96 mmol, 76% yield).

LC-MS (ESI): m/z (M+1): 466.2 (Method 3)

### Intermediate 289: 6-(5-chloro-2-fluorophenyl)-3-({[3-(hydroxymethyl)-2-oxooxolan-3-yl]methyl}(methyl)amino)pyridazine-4-carboxylic acid trifluoroacetic salt

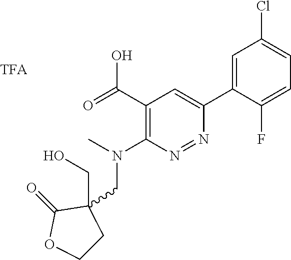 [SMILES: CN(CC1(CO)CCOC1=O)c1nnc(-c2cc(Cl)ccc2F)cc1C(=O)O]

Intermediate 289 was prepared following the procedure used for the synthesis of Intermediate 215, starting from Intermediate 288 (448 mg, 0.95 mmol) to afford title compound (0.95 mmol, quantitative yield). LC-MS (ESI): m/z (M+1): 410.1 (Method 3)

### Intermediate 290: 3-({[4-amino-6-(5-chloro-2-fluorophenyl)pyridazin-3-yl](methyl)amino}methyl)-3-(hydroxymethyl)oxolan-2-one

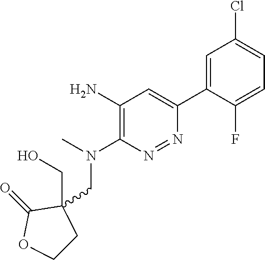 [SMILES: CN(CC1(CO)CCOC1=O)c1nnc(-c2cc(Cl)ccc2F)cc1N]

Intermediate 290 was prepared following the procedure used for the synthesis of Intermediate 231, starting from Intermediate 289 (0.95 mmol) to afford title compound (69 mg, 0.19 mmol, 19% yield). LC-MS (ESI): m/z (M+1): 381.1 (Method 3)

### Intermediate 291: 3-({[4-amino-6-(5-chloro-2-fluorophenyl)pyridazin-3-yl](methyl)amino}methyl)-3-{[(tert-butyldimethylsilyl)oxy]methyl}oxolan-2-one

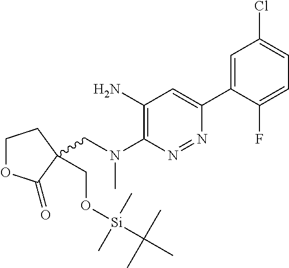 [SMILES: CN(CC1(CO[Si](C)(C)C(C)(C)C)CCOC1=O)c1nnc(-c2cc(Cl)ccc2F)cc1N]

To a stirred mixture of Intermediate 290 (59 mg, 0.15 mmol) and DMAP (3.8 mg, 0.03 mmol) in DCM (2.81 mL) at RT, imidazole (18 mg, 0.26 mmol) was added followed by tert-butyldimethylchlorosilane (35.18 mg, 0.230 mmol) and the resulting reaction mixture was stirred overnight at RT. The mixture was diluted with DCM, washed with a concentrated solution of NaHCO₃ and water, the organic phase was dried over Na₂SO₄, filtered and the solvent removed under reduced pressure. The crude material was purified by flash chromatography on Biotage NH cartridge (from cHex to 30% EtOAc) to afford title compound (32 mg, 0.06 mmol, 42% yield).

LC-MS (ESI): m/z (M+1): 495.2 (Method 3)

### Intermediate 292: N-{4-[(3-{[(3-{[(tert-butyldimethylsilyl)oxy]methyl}-2-oxooxolan-3-yl)methyl](methyl)amino}-6-(5-chloro-2-fluorophenyl)pyridazin-4-yl)amino]pyridin-2-yl}-3-(4-methylpiperazin-1-yl)propanamide

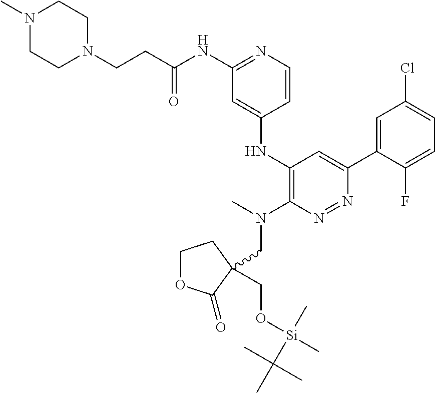 [SMILES: CN1CCN(CCC(=O)Nc2cc(Nc3cc(-c4cc(Cl)ccc4F)nnc3N(C)CC3(CO[Si](C)(C)C(C)(C)C)CCOC3=O)ccn2)CC1]

Intermediate 292 was prepared following the procedure used for the synthesis of Intermediate 189 starting from Intermediate 291 (32 mg, 0.06 mmol) and Intermediate 2 (23 mg, 0.07 mmol) to afford title compound (24 mg, 0.03 mmol, 50% yield). LC-MS (ESI): m/z (M+1): 741.5 (Method 4)

### Intermediate 293: tert-butyl 4-{2-[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]ethyl}piperazine-1-carboxylate

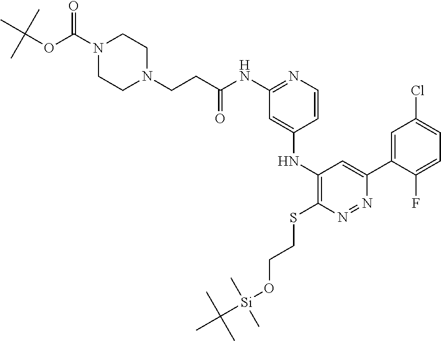 [SMILES: CC(C)(C)OC(=O)N1CCN(CCC(=O)Nc2cc(Nc3cc(-c4cc(Cl)ccc4F)nnc3SCCO[Si](C)(C)C(C)(C)C)ccn2)CC1]

Intermediate 293 was prepared following the procedure used for the synthesis of Intermediate 47 starting from Intermediate 57 (88 mg, 0.21 mmol) and Intermediate 67 (80 mg, 0.19 mmol) to afford title compound (140 mg, 0.19 mmol, 97% yield). LC-MS (ESI): m/z (M+1): 746.1 (Method 2)

### Intermediate 294: Cis {3-[(tert-butyldimethylsilyl)oxy]cyclobutyl}methyl 4-methylbenzene-1-sulfonate

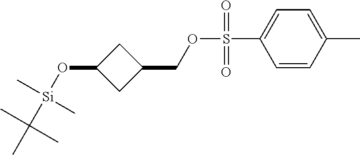 [SMILES: Cc1ccc(S(=O)(=O)OC[C@H]2C[C@@H](O[Si](C)(C)C(C)(C)C)C2)cc1]

To a solution of cis {3-[(tert-butyldimethylsilyl)oxy]cyclobutyl}methanol (Intermediate 154, 830 mg, 3.84 mmol) in DCM (19 mL), TEA was added (1.6 mL, 11.51 mmol) followed by tosyl chloride (1.10 g, 5.75 mmol). The mixture was stirred at RT for 4 hrs. Additional tosyl chloride (439 mg, 2.3 mmol) and TEA (0.53 mL, 3.84 mmol) were added, and the mixture stirred for 3 hrs. The reaction was quenched by adding water, the organic phase separated and washed with brine. The organic phase was dried over Na₂SO₄, filtered, and concentrated under reduced pressure. The crude material was purified by flash chromatography on Biotage Si cartridge (from cHex to 20% EtOAc) to afford title compound (1.44 g, 3.84 mmol, quantitative yield). LC-MS (ESI): m/z (M+1): 371.2 (Method 1)

### Intermediate 295: Cis 1-[({3-[(tert-butyldimethylsilyl)oxy]cyclobutyl}methyl)sulfanyl]ethan-1-one

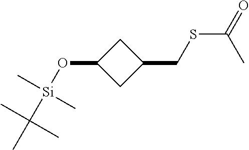 [SMILES: CC(=O)SC[C@H]1C[C@@H](O[Si](C)(C)C(C)(C)C)C1]

A mixture of cis {3-[(tert-butyldimethylsilyl)oxy]cyclobutyl}methyl 4-methylbenzene-1-sulfonate (Intermediate 294, 1.44 g, 3.84 mmol), potassium thioacetate (888 mg, 7.77 mmol) and sodium iodide (58 mg, 0.39 mmol) in DMF (9.7 mL) was stirred at 50° C. for 6 hrs. The mixture was cooled to RT then diluted with EtOAc and washed with sat. aq. NaHCO₃. The organic phase was dried over Na₂SO₄, filtered, and concentrated under reduced pressure. The crude material was purified by flash chromatography on Biotage Si cartridge (from cHex to 10% EtOAc) to afford title compound (646 mg, 2.35 mmol, 61% yield).

LC-MS (ESI): m/z (M+1): 275.2 (Method 1)

### Intermediate 296: Cis 3-[({3-[(tert-butyldimethylsilyl)oxy]cyclobutyl}methyl)sulfanyl]-6-chloropridazin-4-amine

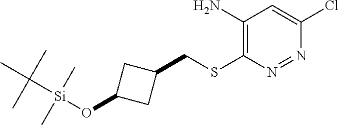 [SMILES: CC(C)(C)[Si](C)(C)O[C@H]1C[C@@H](CSc2nnc(Cl)cc2N)C1]

### Step 1

A mixture of cis 1-[({3-[(tert-butyldimethylsilyl)oxy]cyclobutyl}methyl)sulfanyl]ethan-1-one (Intermediate 295, 646 mg, 2.35 mmol) in THE (15.7 mL) was treated with 2 M lithium aluminium hydride in THE (1.53 mL, 3.06 mmol) at 0° C. and under N₂ atmosphere. The mixture was stirred at the same temperature for 5 minutes, then warmed to RT and stirred for 30 minutes. The reaction was cooled to 0° C. and quenched by adding sat. aq. NaHSO₄. The mixture was extracted with EtOAc and washed with water. The organic phase was dried over Na₂SO₄, filtered and concentrated under reduced pressure to afford cis {3-[(tert-butyldimethylsilyl)oxy]cyclobutyl}methanethiol (530 mg, 2.28 mmol, 97%) that was used as such in the next step.

### Step 2

Intermediate 296 was prepared following the procedure used for the synthesis of Intermediate 176 starting from material (from Step 1, 528 mg, 2.27 mmol) and 3,6-dichloropyridazin-4-amine (250 mg, 1.52 mmol) to afford title compound (495 mg, 1.27 mmol, 84% yield). LC-MS (ESI): m/z (M+1): 360.2 (Method 1)

### Intermediate 297: Cis 3-[({3-[(tert-butyldimethylsilyl)oxy]cyclobutyl}methyl)sulfanyl]-6-(5-chloro-2-fluorophenyl)pyridazin-4-amine

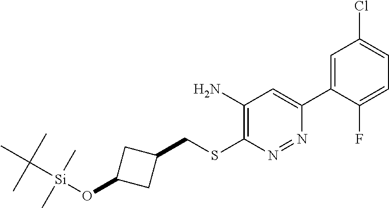 [SMILES: CC(C)(C)[Si](C)(C)O[C@H]1C[C@@H](CSc2nnc(-c3cc(Cl)ccc3F)cc2N)C1]

Intermediate 297 was prepared following the procedure used for the synthesis of Intermediate 8, starting from cis 3-[({3-[(tert-butyldimethylsilyl)oxy]cyclobutyl}methyl)sulfanyl]-6-chloropyridazin-4-amine (Intermediate 296, 495 mg, 1.27 mmol) and 5-chloro-2-fluorobenzeneboronic acid (333 mg, 1.91 mmol) in presence of Pd(dppf)Cl₂ (187 mg, 0.25 mmol) to afford title compound (226 mg, 0.5 mmol, 39% yield). LC-MS (ESI): m/z (M+1): 454.7 (Method 1)

### Intermediate 298: Cis N-[4-({3-[({3-[(tert-butyldimethylsilyl)oxy]cyclobutyl}methyl)sulfanyl]-6-(5-chloro-2-fluorophenyl)pyridazin-4-yl}amino)pyridin-2-yl]-3-(4-methylpiperazin-1-yl)propanamide

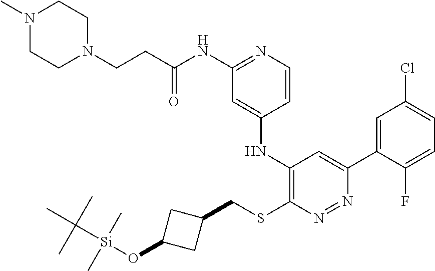 [SMILES: CN1CCN(CCC(=O)Nc2cc(Nc3cc(-c4cc(Cl)ccc4F)nnc3SC[C@H]3C[C@@H](O[Si](C)(C)C(C)(C)C)C3)ccn2)CC1]

Intermediate 298 was prepared following the procedure used for the synthesis of Intermediate 47 starting from cis 3-[({3-[(tert-butyldimethylsilyl)oxy]cyclobutyl}methyl)sulfanyl]-6-(5-chloro-2-fluorophenyl)pyridazin-4-amine (Intermediate 297, 95 mg, 0.21 mmol) and N-(4-bromopyridin-2-yl)-3-(4-methylpiperazin-1-yl)propanamide (Intermediate 2, 78 mg, 0.23 mmol) to afford title compound (91 mg, 0.13 mmol, 62% yield).

LC-MS (ESI): m/z (M+1): 700.4 (Method 2)

### Intermediate 299: N-(4-bromopyridin-2-yl)-3,3-dimethoxycyclobutane-1-carboxamide

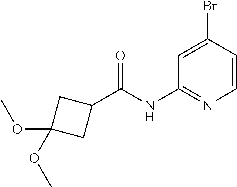 [SMILES: COC1(OC)CC(C(=O)Nc2cc(Br)ccn2)C1]

Intermediate 299 was prepared following the procedure used for the synthesis of Intermediate 171 starting from 4-bromopyridin-2-amine (1.24 g, 7.18 mmol) and 3,3-dimethoxycyclobutane-1-carboxylate methyl ester (500 mg, 2.66 mmol) to afford title compound (480 mg, 1.52 mmol, 57% yield). LC-MS (ESI): m/z (M+1): 315.0 (Method 2)

### Intermediate 300: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3,3-dimethoxycyclobutane-1-carboxamide

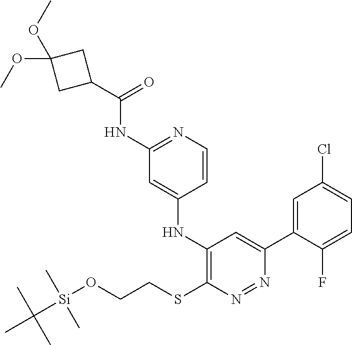 [SMILES: COC1(OC)CC(C(=O)Nc2cc(Nc3cc(-c4cc(Cl)ccc4F)nnc3SCCO[Si](C)(C)C(C)(C)C)ccn2)C1]

Intermediate 300 was prepared following the procedure used for the synthesis of Intermediate 189 starting from Intermediate 299 (42 mg, 0.13 mmol) and Intermediate 67 (50 mg, 0.12 mmol) to afford title compound (67 mg, 0.10 mmol, 86% yield). LC-MS (ESI): m/z (M+1): 648.3 (Method 2)

### Intermediate 301: N-(4-{[6-(5-chloro-2-fluorophenyl)-3-[(2-hydroxyethyl)sulfanyl]pyridazin-4-yl]amino}pyridin-2-yl)-3-oxocyclobutane-1-carboxamide

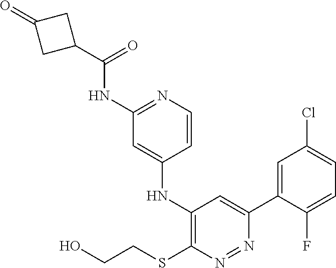 [SMILES: O=C1CC(C(=O)Nc2cc(Nc3cc(-c4cc(Cl)ccc4F)nnc3SCCO)ccn2)C1]

A solution of Intermediate 300 (67.0 mg, 0.10 mmol) in THE (1.03 mL) was treated with aq. HCl (1 N) (1.03 mL, 1.03 mmol) at RT and the solution was stirred overnight. The reaction was quenched by adding sat. aq. NaHCO₃, then extracted with EtOAc. The organic phase was separated, dried over Na₂SO₄, filtered and concentrated under reduced pressure. The crude material was purified by flash chromatography on Biotage Si cartridge (from DCM to 20% MeOH) to afford title compound (30 mg, 0.06 mmol, 59% yield). LC-MS (ESI): m/z (M+1): 488.1 (Method 2)

### Intermediate 302: tert-butyl 8-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}-5,8-diazaspiro[3.5]nonane-5-carboxylate

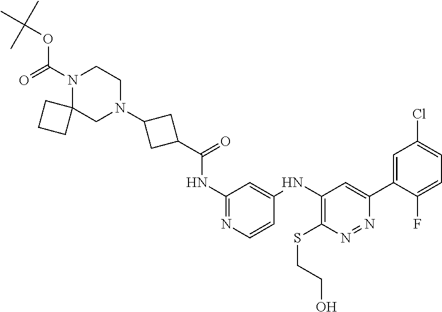 [SMILES: CC(C)(C)OC(=O)N1CCN(C2CC(C(=O)Nc3cc(Nc4cc(-c5cc(Cl)ccc5F)nnc4SCCO)ccn3)C2)CC12CCC2]

Intermediate 302 was prepared following the procedure used for the synthesis of Intermediate 170 starting from tert-butyl 5,8-diazaspiro[3.5]nonane-5-carboxylate (99 mg, 0.44 mmol) and Intermediate 301 (85 mg, 0.17 mmol) to afford title compound (104 mg, 0.15 mmol, 85% yield) as inseparable diasteroisomeric mixture cis and trans. LC-MS (ESI): m/z (M+1): 698.5 (Method 2)

### Intermediate 303: N-(4-{[6-(5-chloro-2-fluorophenyl)-3-[(2-hydroxyethyl)sulfanyl]pyridazin-4-yl]amino}pyridin-2-yl)-3-{5,8-diazaspiro[3.5]nonan-8-yl}cyclobutane-1-carboxamide

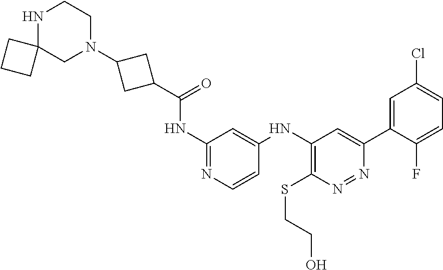 [SMILES: O=C(Nc1cc(Nc2cc(-c3cc(Cl)ccc3F)nnc2SCCO)ccn1)C1CC(N2CCNC3(CCC3)C2)C1]

Intermediate 303 was prepared following the procedure used for the synthesis of Intermediate 40 starting from Intermediate 302 (104 mg, 0.15 mmol) to afford title compound (0.15 mmol, quantitative yield) as inseparable diasteroisomeric mixture cis and trans. LC-MS (ESI): m/z (M+1): 598.4 (Method 2)

### Intermediate 304: tert-butyl 3-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}-3,6-diazabicyclo[3.1.1]heptane-6-carboxylate

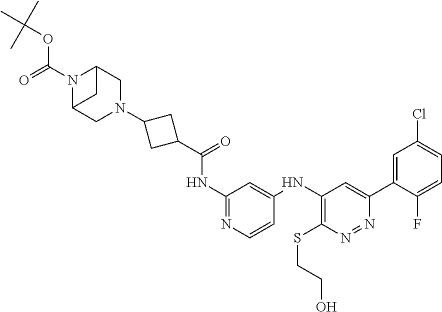 [SMILES: CC(C)(C)OC(=O)N1C2CC1CN(C1CC(C(=O)Nc3cc(Nc4cc(-c5cc(Cl)ccc5F)nnc4SCCO)ccn3)C1)C2]

Intermediate 304 was prepared following the procedure used for the synthesis of Intermediate 170 starting from tert-butyl 3,6-diazabicyclo[3.1.1]heptane-6-carboxylate (86 mg, 0.44 mmol) and Intermediate 301 (85 mg, 0.17 mmol) to afford title compound (113 mg, 0.17 mmol, 97% yield) as inseparable diasteroisomeric mixture cis and trans. LC-MS (ESI): m/z (M+1): 670.5 (Method 2)

### Intermediate 305: N-(4-{[6-(5-chloro-2-fluorophenyl)-3-[(2-hydroxyethyl)sulfanyl]pyridazin-4-yl]amino}pyridin-2-yl)-3-{3,6-diazabicyclo[3.1.1]heptan-3-yl}cyclobutane-1-carboxamide

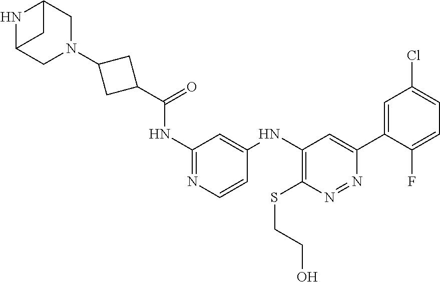 [SMILES: O=C(Nc1cc(Nc2cc(-c3cc(Cl)ccc3F)nnc2SCCO)ccn1)C1CC(N2CC3CC(C2)N3)C1]

Intermediate 305 was prepared following the procedure used for the synthesis of Intermediate 40 starting from Intermediate 304 (113 mg, 0.17 mmol) to afford title compound (0.17 mmol, quantitative yield) as inseparable diasteroisomeric mixture cis and trans. LC-MS (ESI): m/z (M+1): 570.2 (Method 2)

### Intermediate 306: N-(6-{[3-({2-[(tert-butyldimethylsilyl)oxy]ethyl}sulfanyl)-6-(5-chloro-2-fluorophenyl)pyridazin-4-yl]amino}pyrimidin-4-yl)-3-(4-methylpiperazin-1-yl)propanamide

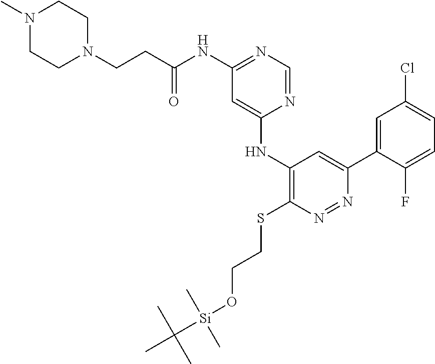 [SMILES: CN1CCN(CCC(=O)Nc2cc(Nc3cc(-c4cc(Cl)ccc4F)nnc3SCCO[Si](C)(C)C(C)(C)C)ncn2)CC1]

Intermediate 306 was prepared following the procedure used for the synthesis of Intermediate 47 starting from Intermediate 272 (137 mg, 0.27 mmol) and Intermediate 67 (100 mg, 0.24 mmol) to afford title compound (30 mg, 0.04 mmol, 19% yield). LC-MS (ESI): m/z (M+1): 661.3 (Method 1)

### Intermediate 307: tert-butyl 4-(2-carbamoylethyl)-2,6-dimethylpiperazine-1-carboxylate

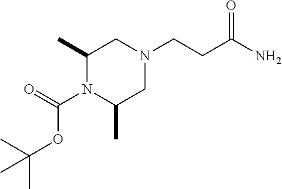 [SMILES: C[C@@H]1CN(CCC(N)=O)C[C@H](C)N1C(=O)OC(C)(C)C]

Intermediate 307 was prepared following the procedure used for the synthesis of Intermediate 271 starting from 2-propenamide (200 mg, 2.81 mmol) and tert-butyl 2,6-dimethylpiperazine-1-carboxylate (603 mg, 2.81 mmol) to afford title compound (770 mg, 2.70 mmol, 96% yield).

### Intermediate 308: tert-butyl 4-{2-[(6-chloropyrimidin-4-yl)carbamoyl]ethyl}-2,6-dimethylpiperazine-1-carboxylate

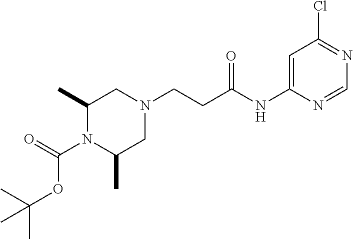 [SMILES: C[C@@H]1CN(CCC(=O)Nc2cc(Cl)ncn2)C[C@H](C)N1C(=O)OC(C)(C)C]

Intermediate 308 was prepared following the procedure used for the synthesis of Intermediate 272 starting from Intemediate 307 (200 mg, 2.81 mmol) and 4,6-dichloropyrimidine (820 mg, 5.5 mmol) to afford title compound (770 mg, 2.70 mmol, 96% yield). LC-MS (ESI): m/z (M+1): 398.4 (Method 2)

### Intermediate 309: tert-butyl 4-{2-[(6-{[3-({2-[(tert-butyldimethylsilyl)oxy]ethyl}sulfanyl)-6-(5-chloro-2-fluorophenyl)pyridazin-4-yl]amino}pyrimidin-4-yl)carbamoyl]ethyl}-2,6-dimethylpiperazine-1-carboxylate

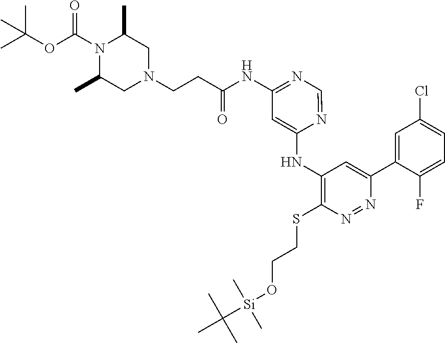 [SMILES: C[C@@H]1CN(CCC(=O)Nc2cc(Nc3cc(-c4cc(Cl)ccc4F)nnc3SCCO[Si](C)(C)C(C)(C)C)ncn2)C[C@H](C)N1C(=O)OC(C)(C)C]

Intermediate 309 was prepared following the procedure used for the synthesis of Intermediate 189 starting from Intermediate 308 (74 mg, 0.19 mmol) and Intermediate 67 (70 mg, 0.17 mmol) to afford title compound (80 mg, 0.10 mmol, 61% yield). LC-MS (ESI): m/z (M+1): 775.5 (Method 2)

### Intermediate 310: tert-butyl 4-{2-[(4-bromopyridin-2-yl)carbamoyl]ethyl}-2,6-dimethylpiperazine-1-carboxylate

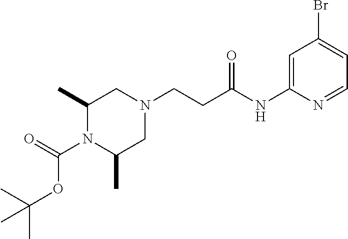 [SMILES: C[C@@H]1CN(CCC(=O)Nc2cc(Br)ccn2)C[C@H](C)N1C(=O)OC(C)(C)C]

Intermediate 310 was prepared following the procedure used for the synthesis of Intermediate 2 starting from Intermediate 1 (700 mg, 3.08 mmol) and tert-butyl 2,6-dimethylpiperazine-1-carboxylate (892 mg, 4.16 mmol) to afford title compound (930 mg, 2.11 mmol, 68% yield).

LC-MS (ESI): m/z (M+1): 441.3 (Method 3)

### Intermediate 311: tert-butyl 4-{2-[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]ethyl}-2,6-dimethylpiperazine-1-carboxylate

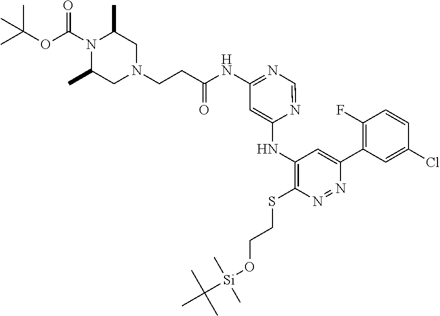 [SMILES: C[C@@H]1CN(CCC(=O)Nc2cc(Nc3cc(-c4cc(Cl)ccc4F)nnc3SCCO[Si](C)(C)C(C)(C)C)ncn2)C[C@H](C)N1C(=O)OC(C)(C)C]

Intermediate 311 was prepared following the procedure used for the synthesis of Intermediate 189 starting from Intermediate 310 (113 mg, 0.25 mmol) and Intermediate 67 (100 mg, 0.23 mmol) to afford title compound (160 mg, 0.21 mmol, 89% yield). LC-MS (ESI): m/z (M+1): 774.5 (Method 2)

### Intermediate 312: 6-chloro-3-{[2-(trimethylsilyl)ethyl]sulfanyl}pyridazin-4-amine

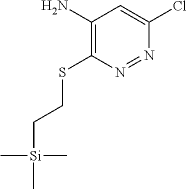

Intermediate 312 was prepared following the procedure used for the synthesis of Intermediate 176, starting from 3,6-dichloropyridazin-4-amine (1 g, 6.10 mmol) and 2-(trimethylsilyl)-ethanethiol (1.27 ml, 7.93 mmol) to afford title compound (1.4 g, 5.35 mmol, 88% yield).

LC-MS (ESI): m/z (M+1): 262.2 (Method 1)

### Intermediate 313: 6-(5-chloro-2-fluorophenyl)-3-{[2-(trimethylsilyl)ethyl]sulfanyl}pyridazin-4-amine

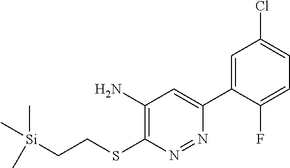 [SMILES: C[Si](C)(C)CCSc1nnc(-c2cc(Cl)ccc2F)cc1N]

Intermediate 313 was prepared following the procedure used for the synthesis of Intermediate 8, starting from Intermediate 312 (500 mg, 1.91 mmol) and 5-chloro-2-fluorobenzeneboronic acid (500 mg, 2.87 mmol) in presence of Pd(dppf)Cl₂ (280 mg, 0.38 mmol) to afford title compound (320 mg, 0.90 mmol, 47% yield).

LC-MS (ESI): m/z (M+1): 356.3 (Method 3)

### Intermediate 314: N-(4-{[6-(5-chloro-2-fluorophenyl)-3-{[2-(trimethylsilyl)ethyl]sulfanyl}pyridazin-4-yl]amino}pyridin-2-yl)-3-(4-methylpiperazin-1-yl)propanamide

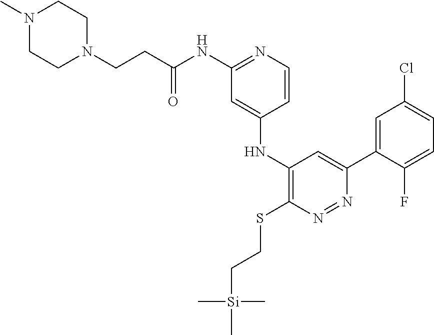 [SMILES: CN1CCN(CCC(=O)Nc2cc(Nc3cc(-c4cc(Cl)ccc4F)nnc3SCC[Si](C)(C)C)ccn2)CC1]

Intermediate 314 was prepared following the procedure used for the synthesis of Intermediate 189 starting from Intermediate 313 (320 mg, 0.90 mmol) and Intermediate 2 (337 mg, 0.99 mmol) to afford title compound (540 mg, 0.89 mmol, 99% yield). LC-MS (ESI): m/z (M+1): 602.3 (Method 4)

### Intermediate 315: methyl 3-[(methanesulfonyloxy)methyl]bicyclo[1.1.1]pentane-1-carboxylate

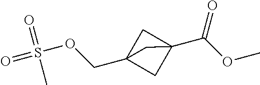

Methanesulfonyl chloride (332 μL, 4.29 mmol) was added to an ice cooled stirred solution of Intermediate 256 (515 mg, 3.30 mmol) and TEA (0.92 mL, 6.59 mmol) in DCM (33 mL). After 1 h at RT, the reaction was diluted with DCM and washed with saturated NaHCO₃ aqueous solution, organic phase was separated, dried over Na₂SO₄, filtered and evaporated to afford title compound 770 mg, 3.30 mmol, quantitative yield).

### Intermediate 316: methyl 3-[(4-methylpiperazin-1-yl)methyl]bicyclo[1.1.1]pentane-1-carboxylate

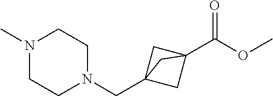

To a stirred solution of Intermediate 315 (770 mg, 3.30 mmol) in MeCN (16.45 mL), 1-methylpiperazine (0.60 mL, 6.57 mmol) and TEA (1.83 mL, 13.15 mmol) were added. The mixture was stirred at 60° C. for 24 hrs. Volatiles were removed under vacuum and the crude material was purified by flash chromatography on Biotage NH cartridge (from DCM to 5% MeOH) affording title compound (678 mg, 2.85 mmol, 87% yield).

### Intermediate 317: N-(4-bromopyridin-2-yl)-3-[(4-methylpiperazin-1-yl)methyl]bicyclo[1.1.1]pentane-1-carboxamide

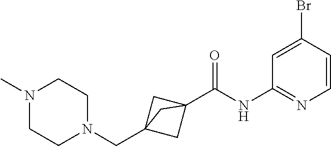 [SMILES: CN1CCN(CC23CC(C(=O)Nc4cc(Br)ccn4)(C2)C3)CC1]

Intermediate 317 was prepared following the procedure used for the synthesis of Intermediate 171 starting from Intermediate 316 (674 mg, 2.83 mmol) to afford title compound (483 mg, 1.27 mmol, 49% yield). LC-MS (ESI): m/z (M+1): 379.1 (Method 2)

### Intermediate 318: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-[(4-methylpiperazin-1-yl)methyl] bicyclo[1.1.1]pentane-1-carboxamide

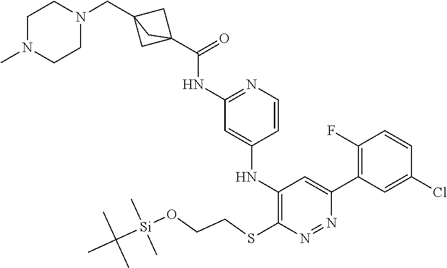 [SMILES: CN1CCN(CC23CC(C(=O)Nc4cc(Nc5cc(-c6cc(Cl)ccc6F)nnc5SCCO[Si](C)(C)C(C)(C)C)ccn4)(C2)C3)CC1]

Intermediate 318 was prepared following the procedure used for the synthesis of Intermediate 189 starting from Intermediate 67 (80 mg, 0.19 mmol) and Intermediate 317 (81 mg, 0.21 mmol) to afford title compound (135 mg, 0.19 mmol, 98% yield). LC-MS (ESI): m/z (M+1): 712.5 (Method 2)

### Intermediate 319: ethyl 3-(4-cyclopropylpiperazin-1-yl)cyclobutane-1-carboxylate

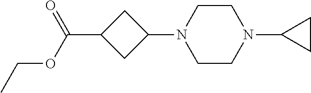 [SMILES: CCOC(=O)C1CC(N2CCN(C3CC3)CC2)C1]

Intermediate 319 was prepared following the procedure used for the synthesis of Intermediate 170 starting from 1-cyclopropylpiperazine (0.73 ml, 5.42 mmol) and ethyl 3-oxocyclobutane-1-carboxylate (700 mg, 4.92 mmol) to afford title compound (530 mg, 2.1 mmol, 43% yield) as inseparable diasteroisomeric mixture cis and trans. LC-MS (ESI): m/z (M+1): 252.6 (Method 2)

### Intermediate 320: Cis N-(6-chloropyrimidin-4-yl)-3-(4-cyclopropylpiperazin-1-yl)cyclobutane-1-carboxamide

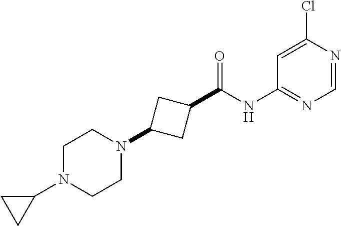 [SMILES: O=C(Nc1cc(Cl)ncn1)[C@H]1C[C@@H](N2CCN(C3CC3)CC2)C1]

Intermediate 320 was prepared following the procedure used for the synthesis of Intermediate 171 starting from Intermediate 319 (536 mg, 2.12 mmol) to afford title compound (234 mg, 0.70 mmol, 36% yield). Only the major isomer cis was isolated. LC-MS (ESI): m/z (M+1): 336.3 (Method 2)

### Intermediate 321: Cis N-(6-{[3-({2-[(tert-butyldimethylsilyl)oxy]ethyl}sulfanyl)-6-(5-chloro-2-fluorophenyl)pyridazin-4-yl]amino}pyrimidin-4-yl)-3-(4-cyclopropylpiperazin-1-yl)cyclobutane-1-carboxamide

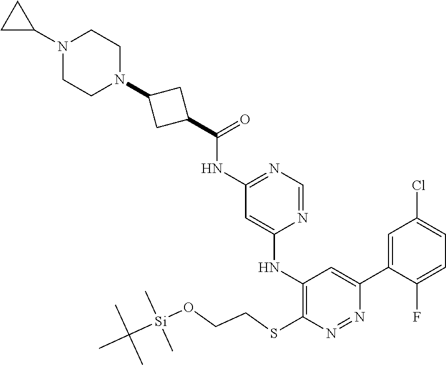

Intermediate 321 was prepared following the procedure used for the synthesis of Intermediate 189 starting from Intermediate 67 (80 mg, 0.19 mmol) and cis N-(6-chloropyrimidin-4-yl)-3-(4-cyclopropylpiperazin-1-yl)cyclobutane-1-carboxamide (Intermediate 320, 91 mg, 0.27 mmol) to afford title compound (110 mg, 0.15 mmol, 80% yield). LC-MS (ESI): m/z (M+1): 713.4 (Method 2)

### Intermediate 322: N-(6-chloropyrimidin-4-yl)-3-[(4-methylpiperazin-1-yl)methyl]bicyclo[1.1.1]pentane-1-carboxamide

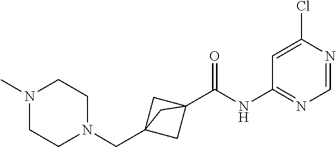 [SMILES: CN1CCN(CC23CC(C(=O)Nc4cc(Cl)ncn4)(C2)C3)CC1]

Intermediate 322 was prepared following the procedure used for the synthesis of Intermediate 171 starting from Intermediate 316 (238 mg, 2.12 mmol) to afford title compound (148 mg, 0.44 mmol, 23% yield). LC-MS (ESI): m/z (M+1): 336.5 (Method 4)

### Intermediate 323: N-(6-{[3-({2-[(tert-butyldimethylsilyl)oxy]ethyl}sulfanyl)-6-(5-chloro-2-fluorophenyl)pyridazin-4-yl]amino}pyrimidin-4-yl)-3-[(4-methylpiperazin-1-yl)methyl]bicyclo[1.1.1]pentane-1-carboxamide

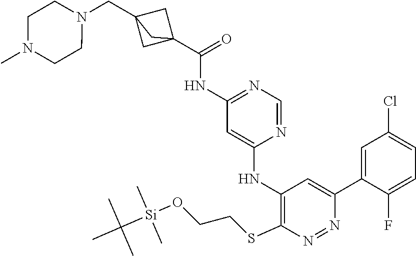 [SMILES: CN1CCN(CC23CC(C(=O)Nc4cc(Nc5cc(-c6cc(Cl)ccc6F)nnc5SCCO[Si](C)(C)C(C)(C)C)ncn4)(C2)C3)CC1]

Intermediate 323 was prepared following the procedure used for the synthesis of Intermediate 189 starting from Intermediate 67 (40 mg, 0.10 mmol) and Intermediate 322 (45 mg, 0.13 mmol) to afford title compound (37 mg, 0.05 mmol, 50% yield). LC-MS (ESI): m/z (M+1): 713.4 (Method 4)

### Intermediate 324: methyl 3-[(4-cyclopropylpiperazin-1-yl)methyl]bicyclo[1.1.1]pentane-1-carboxylate

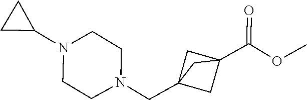 [SMILES: COC(=O)C12CC(CN3CCN(C4CC4)CC3)(C1)C2]

Intermediate 324 was prepared following the procedure used for the synthesis of Intermediate 316 starting from Intermediate 315 (800 mg, 3.41 mmol) and 1-cyclopropylpiperazine (474 mg, 3.76 mmol) to afford title compound (790 mg, 2.99 mmol, 87% yield).

### Intermediate 325: N-(4-bromopyridin-2-yl)-3-[(4-cyclopropylpiperazin-1-yl)methyl]bicyclo[1.1.1]pentane-1-carboxamide

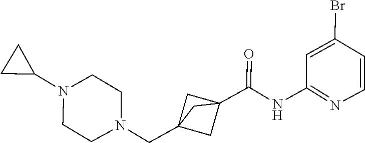 [SMILES: O=C(Nc1cc(Br)ccn1)C12CC(CN3CCN(C4CC4)CC3)(C1)C2]

Intermediate 325 was prepared following the procedure used for the synthesis of Intermediate 171 starting from 4-bromopyridin-2-amine (460 mg, 2.66 mmol) and Intermediate 324 (772 mg, 2.93 mmol) to afford title compound (244 mg, 0.6 mmol, 23% yield). LC-MS (ESI): m/z (M+1): 405.1 (Method 4)

### Intermediate 326: N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-[(4-cyclopropylpiperazin-1-yl)methyl]bicyclo[1.1.1]pentane-1-carboxamide

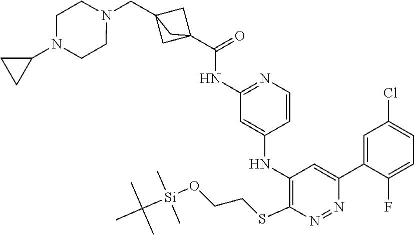 [SMILES: CC(C)(C)[Si](C)(C)OCCSc1nnc(-c2cc(Cl)ccc2F)cc1Nc1ccnc(NC(=O)C23CC(CN4CCN(C5CC5)CC4)(C2)C3)c1]

Intermediate 326 was prepared following the procedure used for the synthesis of Intermediate 189 starting from Intermediate 67 (80 mg, 0.19 mmol) and Intermediate 325 (123 mg, 0.31 mmol) to afford title compound (125 mg, 0.17 mmol, 87% yield). LC-MS (ESI): m/z (M+1): 738.4 (Method 4)

### Intermediate 327: tert-butyl 4-{2-[(4-{[6-(5-chloro-2-fluorophenyl)-3-{2-[(propan-2-yloxy)carbonyl]azetidin-1-yl}pyridazin-4-yl]amino}pyridin-2-yl)carbamoyl]ethyl}-2,6-dimethylpiperazine-1-carboxylate

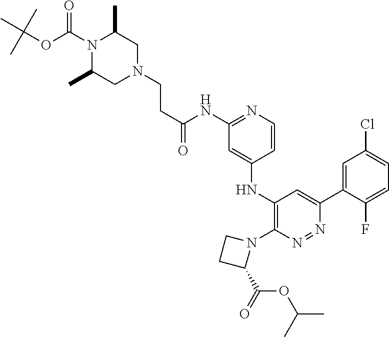

Intermediate 327 was prepared following the procedure used for the synthesis of Intermediate 189 starting from Intermediate 285 (160 mg, 0.44 mmol) and Intermediate 310 (252 mg, 0.57 mmol) at 80° C. to afford title compound (30 mg, 0.04 mmol, 9% yield). LC-MS (ESI): m/z (M+1): 725.4 (Method 4)

### Intermediate 328: tert-butyl 4-[13-(ethoxycarbonyl)cyclobutyl]-2,6-dimethylpiperazine-1-carboxylate

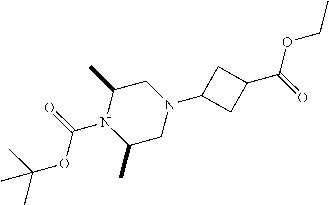

Intermediate 328 was prepared following the procedure used for the synthesis of Intermediate 170 starting from tert-butyl 2,6-dimethylpiperazine-1-carboxylate (66 g, 7.74 mmol) and ethyl 3-oxocyclobutane-1-carboxylate (1 g, 7.03 mmol) to afford title compound (1.18 g, 3.48 mmol, 49% yield) as inseparable diasteroisomeric mixture cis and trans.

### Intermediate 329: tert-butyl 4-{3-[(6-chloropyrimidin-4-yl)carbamoyl]cyclobutyl}-2,6-dimethylpiperazine-1-carboxylate

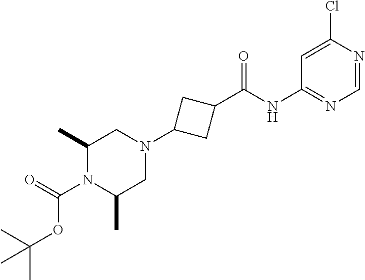

Intermediate 329 was prepared following the procedure used for the synthesis of Intermediate 171 starting from 6-chloro-4-pyrimidinamine (725 mg, 5.60 mmol) and cis tert-butyl 4-[3-(ethoxycarbonyl)cyclobutyl]-2,6-dimethylpiperazine-1-carboxylate (Intermediate 328, 1.18 g, 3.48 mmol) to afford title compound (1.24 g, 2.41 mmol, 69% yield) as inseparable diasteroisomeric mixture cis and trans.

LC-MS (ESI): m/z (M+1): 424.6 (Method 4)

### Intermediate 330: tert-butyl 4-{3-[(6-{[6-(5-chloro-2-fluorophenyl)-3-{methyl[(3-methyl-2-oxooxolan-3-yl)methyl]amino}pyridazin-4-yl]amino}pyrimidin-4-yl)carbamoyl]cyclobutyl}-2,6-dimethylpiperazine-1-carboxylate

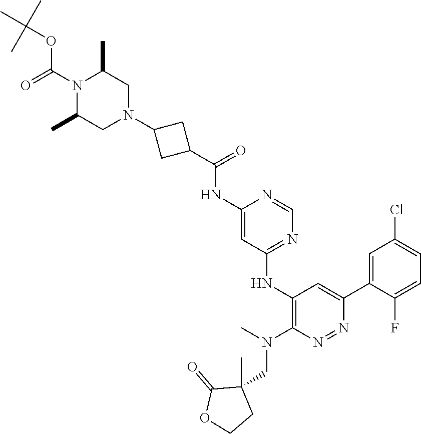

Intermediate 330 was prepared following the procedure used for the synthesis of Intermediate 189 starting from Intermediate 266 (170 mg, 0.47 mmol) and Intermediate 329 (218 mg, 0.49 mmol) at 80° C. to afford title compound (133 mg, 0.18 mmol, 38% yield) as inseparable diasteroisomeric mixture cis and trans.

LC-MS (ESI): m/z (M+1): 752.5 (Method 4)

### Intermediate 331 (cis Enantiomer 1) and Intermediate 332 (trans Enantiomer N-(4-bromopyridin-2-yl)-3-(4-methylpiperazin-1-yl)cyclopentane-1-carboxamide

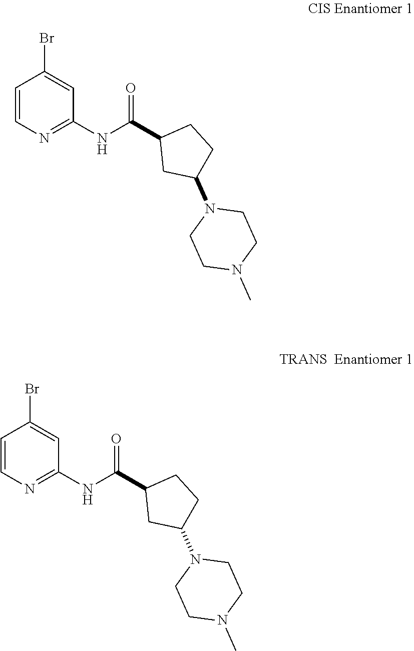

### Step 1

1-Methylpiperazine (0.86 mL, 7.74 mmol) and 3-oxocyclopentanecarboxylic acid methyl ester (1000 mg, 7.03 mmol) were mixed in DCM (20 mL) and stirred for 15 min at RT. Sodium triacetoxyborohydride (2.98 g, 14.7 mmol) was added portion-wise and the resulting reaction mixture was stirred overnight at RT. EtOAc (10 mL) was added carefully and the mixture was stirred for 30 min, then it was concentrated under reduced pressure. The crude material was dissolved in MeOH and the solution was charged onto a SCX, washed with MeOH, and eluted with 1 N NH₃ in MeOH. Evaporation of fractions afforded a crude material that was purified by flash chromatography on Biotage silica NH cartridge (from cHex to 30% EtOAc) to afford methyl 3-(4-methylpiperazin-1-yl)cyclopentane-1-carboxylate (1.12 g, 4.95 mmol, 70% yield) as inseparable mixture of racemic cis and trans diasteroisomers.

### Step 2

Intermediates 331 and 332 were prepared following the procedure used for the synthesis of Intermediate 171 starting from 4-bromopyridin-2-amine (2.1 g, 4.86 mmol) and using the cis and trans mixture of methyl 3-(4-methylpiperazin-1-yl)cyclopentane-1-carboxylate (from Step 1, 1.10 g, 4.86 mmol) to afford 480 mg (1.31 mmol, 29% yield) as inseparable mixture of racemic cis and trans diasteroisomers. The mixture was separated into the single diasteroisomers by preparative chiral HPLC.

### Conditions:

| Column | Chiralpak AD-H (25 × 3.0 cm), 5μ | |
|---|---|---|
| Mobile phase | n-Hexane/(Ethanol + 0.1% isopropylamine) | |
| | 70/30% v/v | |
| Flow rate (ml/min) | 38 | ml/min |
| DAD detection | 220 | nm |
| Loop | 800 | μL |

Intermediate 331 (cis Enantiomer 1) was obtained as first eluted enantioenriched diasteroisomer (68 mg)
Rt.=9.1 min, de 100%, ee 100%
LC-MS (ESI): m/z (M+1): 368.7 (Method 4)
Intermediate 332 (trans Enantiomer 1) was obtained as second eluted enantioenriched diasteroisomer (67 mg)
Rt.=12.9 min, de 99%, ee 99%
LC-MS (ESI): m/z (M+1): 368.7 (Method 4)
With this method cis Enantiomer 2 and trans Enantiomer 2 were collected together (150 mg) and no further processed.

### Intermediate 333: Cis Enantiomer 1 N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)cyclopentane-1-carboxamide

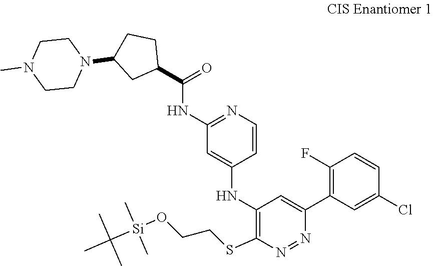 [SMILES: CN1CCN([C@H]2CC[C@@H](C(=O)Nc3cc(Nc4cc(-c5cc(Cl)ccc5F)nnc4SCCO[Si](C)(C)C(C)(C)C)ccn3)C2)CC1]

Intermediate 333 was prepared following the procedure used for the synthesis of Intermediate 189 starting from Intermediate 67 (82 mg, 0.20 mmol) and cis Enantiomer 1 N-(4-bromopyridin-2-yl)-3-(4-methylpiperazin-1-yl)cyclopentane-1-carboxamide (Intermediate 331, 68 mg, 0.18 mmol) to afford title compound (120 mg, 0.17 mmol, 95% yield). LC-MS (ESI): m/z (M+1): 700.5 (Method 4)

### Intermediate 334: Trans Enantiomer 1 N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)cyclopentane-1-carboxamide

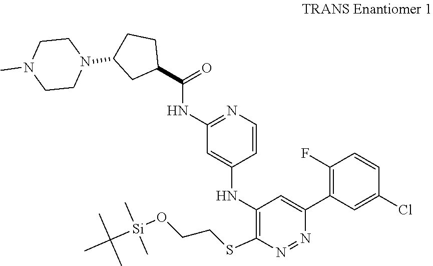 [SMILES: CN1CCN([C@@H]2CC[C@@H](C(=O)Nc3cc(Nc4cc(-c5cc(Cl)ccc5F)nnc4SCCO[Si](C)(C)C(C)(C)C)ccn3)C2)CC1]

Intermediate 334 was prepared following the procedure used for the synthesis of Intermediate 189 starting from Intermediate 67 (83 mg, 0.20 mmol) and Intermediate 332 (67 mg, 0.18 mmol) to afford title compound (100 mg, 0.14 mmol, 78% yield). LC-MS (ESI): m/z (M+1): 700.5 (Method 4)

### Intermediate 335: tert-butyl 4-{[3-(methoxycarbonyl)bicyclo[1.1.1]pentan-1-yl]methyl}-2,6-dimethylpiperazine-1-carboxylate

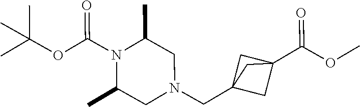

Intermediate 335 was prepared following the procedure used for the synthesis of Intermediate 316 starting from Intermediate 315 (520 mg, 2.22 mmol) and tert-butyl 2,6-dimethylpiperazine-1-carboxylate (76 mg, 2.22 mmol) to afford title compound (310 mg, 0.88 mmol, 40% yield).

### Intermediate 336: tert-butyl 4-({3-[(4-bromopyridin-2-yl)carbamoyl]bicyclo[1.1.1]pentan-1-yl}methyl)-2,6-dimethylpiperazine-1-carboxylate

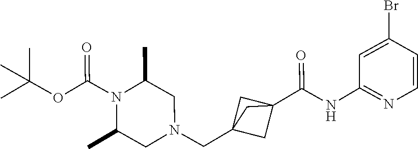

Intermediate 336 was prepared following the procedure used for the synthesis of Intermediate 171 starting from 4-bromopyridin-2-amine (243 mg, 1.40 mmol) and Intermediate 335 (310 mg, 0.88 mmol) to afford title compound (319 mg, 0.65 mmol, 73% yield). LC-MS (ESI): m/z (M+1): 493.3 (Method 4)

### Intermediate 337: tert-butyl 4-({3-[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]bicyclo[1.1.1]pentan-1-yl}methyl)-2,6-dimethylpiperazine-1-carboxylate

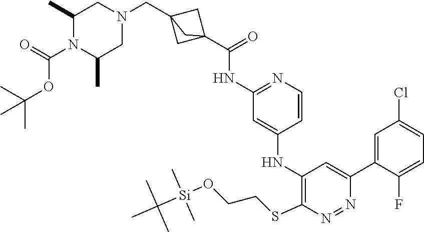 [SMILES: C[C@@H]1CN(CC23CC(C(=O)Nc4cc(Nc5cc(-c6cc(Cl)ccc6F)nnc5SCCO[Si](C)(C)C(C)(C)C)ccn4)(C2)C3)C[C@H](C)N1C(=O)OC(C)(C)C]

Intermediate 337 was prepared following the procedure used for the synthesis of Intermediate 189 starting from Intermediate 67 (80 mg, 0.19 mmol) and Intermediate 336 (105 mg, 0.21 mmol) to afford title compound (155 mg, 0.19 mmol, 97% yield). LC-MS (ESI): m/z (M+1): 826.5 (Method 4)

### Intermediate 338: tert-butyl 4-{[(4-bromopyridin-2-yl)carbamoyl]methyl}-2,6-dimethylpiperazine-1-carboxylate

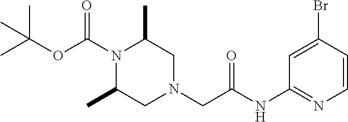

Intermediate 338 was prepared following the procedure used for the synthesis of Intermediate 72 starting from Intermediate 33 (350 mg, 1.40 mmol) and tert-butyl 2,6-dimethylpiperazine-1-carboxylate (91 mg, 1.82 mmol) to afford title compound (1.40 mmol, quantitative yield). LC-MS (ESI): m/z (M+1): 427.3 (Method 4)

### Intermediate 339: tert-butyl 4-{[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]methyl}-2,6-dimethylpiperazine-1-carboxylate

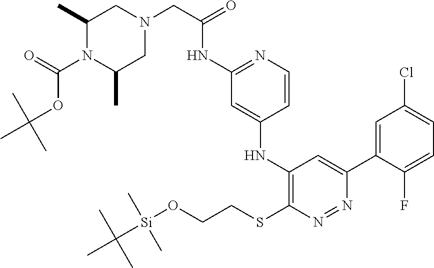 [SMILES: C[C@@H]1CN(CC(=O)Nc2cc(Nc3cc(-c4cc(Cl)ccc4F)nnc3SCCO[Si](C)(C)C(C)(C)C)ccn2)C[C@H](C)N1C(=O)OC(C)(C)C]

Intermediate 339 was prepared following the procedure used for the synthesis of Intermediate 189 starting from Intermediate 67 (95 mg, 0.23 mmol) and Intermediate 338 (120 mg, 0.25 mmol) to afford title compound (0.23 mmol, quantitative yield). LC-MS (ESI): m/z (M+1): 760.6 (Method 4)

### Intermediate 340: 2-{2-[(tert-butyldimethylsilyl)oxy]ethoxy}ethane-1-thiol

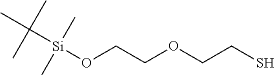

Intermediate 340 was prepared following the procedure used for the synthesis of Intermediate 65 starting from 2-mercaptoethoxy ethanol (2.00 g, 16.37 mmol) to afford title compound (3.55 g, 15.01 mmol, 92% yield).

### Intermediate 341: 3-[(2-{2-[(tert-butyldimethylsilyl)oxy]ethoxy}ethyl)sulfanyl]-6-chloropyridazin-4-amine

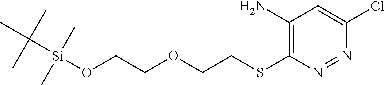 [SMILES: CC(C)(C)[Si](C)(C)OCCOCCSc1nnc(Cl)cc1N]

Intermediate 341 was prepared following the procedure used for the synthesis of Intermediate 61 starting from 3,6-dichloropyridazin-4-amine (930 mg, 5.67 mmol) and Intermediate 340 (2.01 g, 8.51 mmol) to afford title compound (1.59 g, 4.37 mmol, 77% yield). LC-MS (ESI): m/z (M+1): 364.2 (Method 3)

### Intermediate 342: 3-[(2-{2-[(tert-butyldimethylsilyl)oxy]ethoxy}ethyl)sulfanyl]-6-(5-chloro-2-fluorophenyl)pyridazin-4-amine

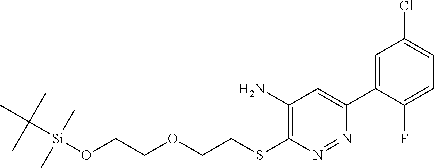 [SMILES: CC(C)(C)[Si](C)(C)OCCOCCSc1nnc(-c2cc(Cl)ccc2F)cc1N]

Intermediate 342 was prepared following the procedure used for the synthesis of Intermediate 8, starting from Intermediate 341 (1.59 g, 4.37 mmol) and 5-chloro-2-fluorobenzeneboronic acid (1.14 g, 6.56 mmol) in presence of Pd(dppf)Cl₂ (640 mg, 0.87 mmol) to afford title compound (1.1 g, 2.40 mmol, 55% yield).

LC-MS (ESI): m/z (M+1): 458.2 (Method 3)

### Intermediate 343: N-[4-({3-[(2-{2-[(tert-butyldimethylsilyl)oxy]ethoxy}ethyl)sulfanyl]-6-(5-chloro-2-fluorophenyl)pyridazin-4-yl}amino)pyridin-2-yl]-3-(4-methylpiperazin-1-yl)propanamide

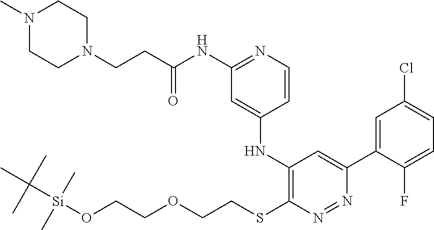 [SMILES: CN1CCN(CCC(=O)Nc2cc(Nc3cc(-c4cc(Cl)ccc4F)nnc3SCCOCCO[Si](C)(C)C(C)(C)C)ccn2)CC1]

Intermediate 343 was prepared following the procedure used for the synthesis of Intermediate 189 starting from Intermediate 342 (100 mg, 0.22 mmol) and Intermediate 2 (90 mg, 0.26 mmol) to afford title compound (139 mg, 0.20 mmol, 90% yield). LC-MS (ESI): m/z (M+1): 704.4 (Method 4)

### Intermediate 344: tert-butyl 4-{2-[(6-{[6-(5-chloro-2-fluorophenyl)-3-{[2-(trimethylsilyl)ethyl]sulfanyl}pyridazin-4-yl]amino}pyrimidin-4-yl)carbamoyl]ethyl}-2,6-dimethylpiperazine-1-carboxylate

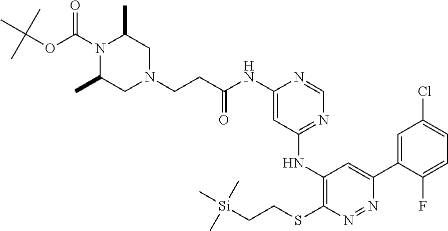 [SMILES: C[C@@H]1CN(CCC(=O)Nc2cc(Nc3cc(-c4cc(Cl)ccc4F)nnc3SCC[Si](C)(C)C)ncn2)C[C@H](C)N1C(=O)OC(C)(C)C]

Intermediate 344 was prepared following the procedure used for the synthesis of Intermediate 189 starting from Intermediate 313 (120 mg, 0.34 mmol) and Intermediate 308 (161 mg, 0.40 mmol) to afford title compound (0.34 mmol, quantitative yield). LC-MS (ESI): m/z (M+1): 717.5 (Method 4)

### Intermediate 345: 3-[(benzyloxy)methyl]-3-methyloxolan-2-one

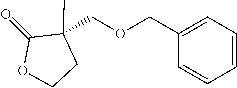 [SMILES: C[C@@]1(COCc2ccccc2)CCOC1=O]

To a stirred solution of 3-methyloxolan-2-one (0.96 ml, 10 mmol) in THE (30 mL), at −78° C. and under N₂, a solution of lithium bis(trimethylsilyl)amide 1M in THE (13 mL, 13 mmol) was added dropwise. The reaction mixture was stirred at −78° C. for 50 min, then benzyl chloromethyl ether (3.2 ml, 23 mmol) was added dropwise. The reaction mixture was stirred for 10 min at −78° C., then was slowly allowed to reach RT and stirred for 3 hrs. The reaction mixture was quenched with EtOAc, then volatiles were removed under vacuum. The crude material was purified was purified by flash chromatography on Biotage silica cartridge (from cHex to 25% EtOAc) to afford title compound (2 g, 9 mmol, 90% yield).

LC-MS (ESI): m/z (M+1): 221.2 (Method 3)

### Intermediate 346: 3-(hydroxymethyl)-3-methyloxolan-2-one

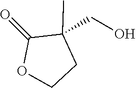

To a stirred solution of 3-[(benzyloxy)methyl]-3-methyloxolan-2-one (Intermediate 345, 1 g, 4.54 mmol) in EtOAc (20 mL), at RT, 10% Pd over carbon 55-65% wet (160 mg, 0.15 mmol) was added and the resulting mixture was hydrogenated at atmospheric pressure for 6 hrs. The mixture was filtered over celite and concentrated under reduced pressure to afford title compound (4.54 mmol, quantitative yield).

### Intermediate 347: tert-butyl N-[(tert-butoxy)carbonyl]-N-[6-(5-chloro-2-fluorophenyl)-3-{[2-(trimethylsilyl)ethyl]sulfanyl}pyridazin-4-yl]carbamate

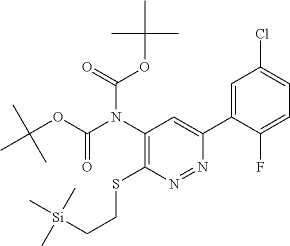 [SMILES: CC(C)(C)OC(=O)N(C(=O)OC(C)(C)C)c1cc(-c2cc(Cl)ccc2F)nnc1SCC[Si](C)(C)C]

Di-tert-butyl dicarbonate (920 mg, 4.21 mmol) was added to a stirred solution of Intermediate 313 (500 mg, 1.4 mmol) and TEA (0.59 mL, 4.21 mmol) in DCM (10 mL) at RT. Then DMAP (34 mg, 0.28 mmol) was added and the mixture was stirred at RT for 6 hrs. The reaction solution was washed with saturated NH₄Cl solution, the organic phase was dried and evaporated. The crude material was purified by flash chromatography on Biotage silica cartridge (from cHex to 10% EtOAc) to afford title compound (770 mg, 1.38 mmol, 98% yield).

LC-MS (ESI): m/z (M+1): 556.3 (Method 4)

### Intermediate 348: tert-butyl N-[(tert-butoxy)carbonyl]-N-[6-(5-chloro-2-fluorophenyl)-3-sulfanylpyridazin-4-yl]carbamate

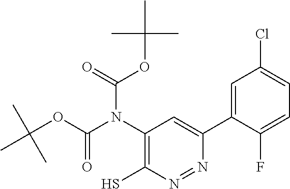 [SMILES: CC(C)(C)OC(=O)N(C(=O)OC(C)(C)C)c1cc(-c2cc(Cl)ccc2F)nnc1S]

Tetrabutylammonium fluoride 1M in THE (1.52 mL, 1.52 mmol) was added dropwise to a solution of Intermediate 347 (770 mg, 1.38 mmol) in THE (7.6 mL) and stirred at RT for 36 hrs. Volatiles were removed under vacuum. The crude material was purified by flash chromatography on Biotage silica cartridge (from cHex to 20% EtOAc) to afford title compound (130 mg, 0.28 mmol, 20% yield).

LC-MS (ESI): m/z (M−1): 454.3 (Method 4)

### Intermediate 349: tert-butyl N-[(tert-butoxy)carbonyl]-N-[6-(5-chloro-2-fluorophenyl)-3-{[(3-methyl-2-oxooxolan-3-yl)methyl]sulfanyl}pyridazin-4-yl]carbamate

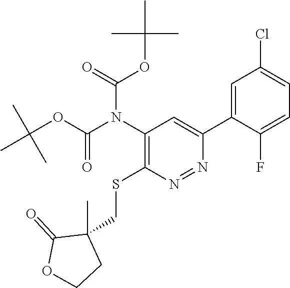 [SMILES: CC(C)(C)OC(=O)N(C(=O)OC(C)(C)C)c1cc(-c2cc(Cl)ccc2F)nnc1SC[C@]1(C)CCOC1=O]

Diisopropyl azodicarboxylate (0.02 mL, 0.10 mmol) was added dropwise to a stirred mixture of Intermediate 346 (13 mg, 0.10 mmol), Intermediate 348 (30 mg, 0.07 mmol) and PPh₃ (27 mg, 0.10 mmol) in THE (1 mL) at 0° C., then the reaction mixture was heated at 50° C. for 40 min. Volatiles were removed under reduced pressure and the obtained crude was purified by flash chromatography on Biotage silica cartridge (from cHex to 20% EtOAc) to afford title compound (20 mg, 0.035 mmol, 53% yield). LC-MS (ESI): m/z (M+1): 568.3 (Method 4)

### Intermediate 350: 3-({[4-amino-6-(5-chloro-2-fluorophenyl)pyridazin-3-yl]sulfanyl}methyl)-3-methyloxolan-2-one

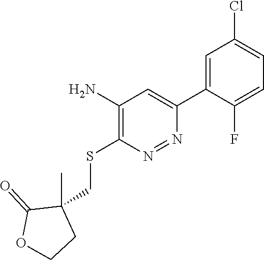 [SMILES: C[C@@]1(CSc2nnc(-c3cc(Cl)ccc3F)cc2N)CCOC1=O]

TFA (0.05 mL, 0.70 mmol) was added to a stirred solution of Intermediate 349 (20 mg, 0.035 mmol) in DCM (2 mL). The mixture was stirred for 4 hrs at RT, then it was diluted with DCM and saturated NaHCO₃ solution. The organic phase was dried and evaporated to afford title compound (11 mg, 0.03 mmol, 85% yield).

LC-MS (ESI): m/z (M+1): 368.3 (Method 4)

### Intermediate 351: tert-butyl 4-{2-[(6-{[6-(5-chloro-2-fluorophenyl)-3-{methyl[(3-methyl-2-oxooxolan-3-yl)methyl]amino}pyridazin-4-yl]amino}pyrimidin-4-yl)carbamoyl]ethyl}-2,6-dimethylpiperazine-1-carboxylate

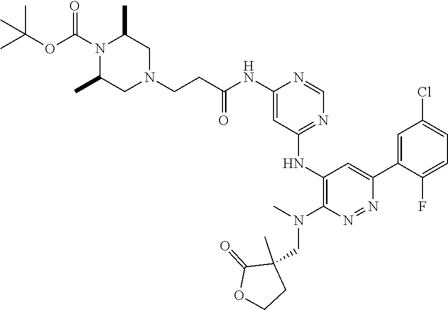 [SMILES: C[C@@H]1CN(CCC(=O)Nc2cc(Nc3cc(-c4cc(Cl)ccc4F)nnc3N(C)C[C@]3(C)CCOC3=O)ncn2)C[C@H](C)N1C(=O)OC(C)(C)C]

Intermediate 351 was prepared following the procedure used for the synthesis of Intermediate 189 starting from Intermediate 266 (83 mg, 0.23 mmol) and Intermediate 308 (96 mg, 0.24 mmol) to afford title compound (108 mg, 0.15 mmol, 65% yield). LC-MS (ESI): m/z (M+1): 726.4 (Method 3)

### Intermediate 352: 3-{[benzyl(methyl)amino]methyl}-3-(methoxymethyl)oxolan-2-one

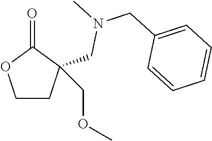 [SMILES: COC[C@@]1(CN(C)Cc2ccccc2)CCOC1=O]

Intermediate 352 was prepared following the procedure used for the synthesis of Intermediate 261, starting from Intermediate 260 (3.5 g, 14.4 mmol) and bromomethyl methyl ether (2.35 ml, 28.7 mmol) to afford title compound (1.70 g, 6.47 mmol, 45% yield). LC-MS (ESI): m/z (M+1): 264.3 (Method 4)

### Intermediate 353: 3-(methoxymethyl)-3-[(methylamino)methyl]oxolan-2-one

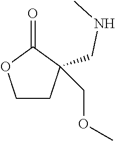

Intermediate 353 was prepared following the procedure used for the synthesis of Intermediate 346, starting from Intermediate 352 (2.00 g, 7.6 mmol) to afford title compound (1.20 g, 6.93 mmol, 91% yield). LC-MS (ESI): m/z (M+1): 174.5 (Method 4)

### Intermediate 354: tert-butyl 6-chloro-3-({[3-(methoxymethyl)-2-oxooxolan-3-yl]methyl}(methyl)amino)pyridazine-4-carboxylate

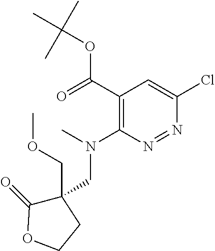 [SMILES: COC[C@@]1(CN(C)c2nnc(Cl)cc2C(=O)OC(C)(C)C)CCOC1=O]

Intermediate 354 was prepared following the procedure used for the synthesis of Intermediate 94 starting from Intermediate 212 (1.00 g, 3.73 mmol) and Intermediate 353 (1.10 g, 6.35 mmol) to afford title compound (550 mg, 1.42 mmol, 38% yield). LC-MS (ESI): m/z (M+1): 386.4 (Method 4)

### Intermediate 355: tert-butyl 6-(5-chloro-2-fluorophenyl)-3-({[3-(methoxymethyl)-2-oxooxolan-3-yl]methyl}(methyl)amino)pyridazine-4-carboxylate

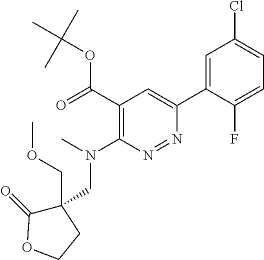 [SMILES: COC[C@@]1(CN(C)c2nnc(-c3cc(Cl)ccc3F)cc2C(=O)OC(C)(C)C)CCOC1=O]

Intermediate 355 was prepared following the procedure used for the synthesis of Intermediate 8, starting from Intermediate 354 (490 mg, 1.27 mmol) and 5-chloro-2-fluorobenzeneboronic acid (443 mg, 2.54 mmol) in presence of Pd(dppf)Cl₂ (186 mg, 0.26 mmol) to afford title compound (530 mg, 1.10 mmol, 87% yield).

LC-MS (ESI): m/z (M+1): 480.3 (Method 4)

### Intermediate 356: 6-(5-chloro-2-fluorophenyl)-3-({[3-(methoxymethyl)-2-oxooxolan-3-yl]methyl}(methyl)amino)pyridazine-4-carboxylic acid trifluoroacetic acid salt

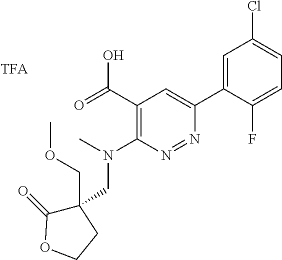 [SMILES: COC[C@@]1(CN(C)c2nnc(-c3cc(Cl)ccc3F)cc2C(=O)O)CCOC1=O]

Intermediate 356 was prepared following the procedure used for the synthesis of Intermediate 215, starting from Intermediate 355 (530 mg, 1.10 mmol) to afford title compound (1.10 mmol, quantitative yield). LC-MS (ESI): m/z (M+1): 424.3 (Method 4)

### Intermediate 357: 3-({[4-amino-6-(5-chloro-2-fluorophenyl)pyridazin-3-yl](methyl)amino}methyl)-3-(methoxymethyl)oxolan-2-one

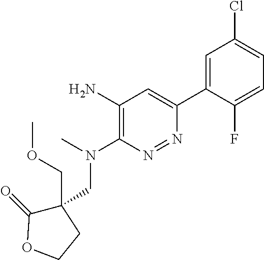 [SMILES: COC[C@@]1(CN(C)c2nnc(-c3cc(Cl)ccc3F)cc2N)CCOC1=O]

Intermediate 357 was prepared following the procedure used for the synthesis of Intermediate 231, starting from Intermediate 356 (1.10 mmol) to afford title compound (300 mg, 0.76 mmol, 69% yield). LC-MS (ESI): m/z (M+1): 395.4 (Method 4)

### Intermediate 358: 6-(iodomethyl)oxan-2-one

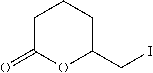

A mixture of 5-hexenoic acid (400 mg, 3.5 mmol) in H₂O (23.4 mL) was sequentially treated with NaHCO₃ (589 mg, 7.01 mmol), sodium iodide (2.10 g, 14.02 mmol) and copper sulfate (2.24 g, 14.02 mmol) to give a slurry which was stirred for 1 h. The mixture was filtered with suction, the filtrated was poured in saturated aqueous Na₂S₂O₈ solution and then extracted with DCM. The organic phase was separated, dried over Na₂SO₄, filtered, and concentrated under reduced pressure to afford title compound (487 mg, 2.03, 58% yield).

### Intermediate 359: tert-butyl N-{2-[(4-amino-6-chloropyridazin-3-yl)sulfanyl]ethyl}carbamate

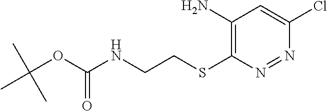 [SMILES: CC(C)(C)OC(=O)NCCSc1nnc(Cl)cc1N]

To an ice cooled solution of 2-(Boc-amino)ethanethiol (2.16 g, 12.2 mmol) in DMF (19.5 ml), NaH 60% dispersion in oil (488 mg, 12.2 mmol) was added and the mixture was stirred at RT for 2 hrs, before slowly adding 3,6-dichloropyridazin-4-amine (1 g, 6.1 mmol) dissolved in DMF (4.88 ml). The reaction was stirred at RT for 3 hrs, then it was diluted with saturated NaHCO₃ aqueous solution and EtOAc. Phases were separated, the organic phase was washed with saturated NaHCO₃ aqueous solution (2×). The organic phase was dried over Na₂SO₄, filtered, and concentrated under vacuum. DCM was added to the crude material, a precipitated was formed, and then, it was filtered to give title compound (1.44 g, 4.71 mmol, 77% yield). LC-MS (ESI): m/z (M+1): 305.1 (Method 3)

### Intermediate 360: tert-butyl N-(2-{[4-amino-6-(5-chloro-2-fluorophenyl)pyridazin-3-yl]sulfanyl}ethyl)carbamate

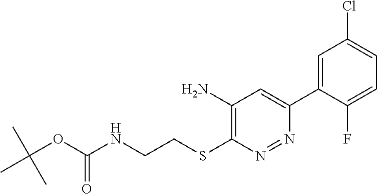 [SMILES: CC(C)(C)OC(=O)NCCSc1nnc(-c2cc(Cl)ccc2F)cc1N]

Intermediate 360 was prepared following the procedure used for the synthesis of Intermediate 8, starting from Intermediate 359 (1.43 g, 4.70 mmol) and 5-chloro-2-fluorobenzeneboronic acid (1.23 g, 7.04 mmol) in presence of Pd(dppf)Cl₂ (686 mg, 0.94 mmol) to afford title compound (1.10 g, 2.76 mmol, 59% yield).

LC-MS (ESI): m/z (M+1): 399.2 (Method 3)

### Intermediate 361: tert-butyl N-(2-{[6-(5-chloro-2-fluorophenyl)-4-({2-[3-(4-methylpiperazin-1-yl)propanamido]pyridin-4-yl}amino)pyridazin-3-yl]sulfanyl}ethyl)carbamate

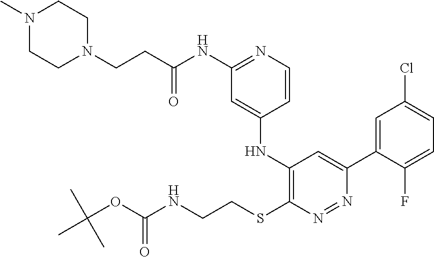 [SMILES: CN1CCN(CCC(=O)Nc2cc(Nc3cc(-c4cc(Cl)ccc4F)nnc3SCCNC(=O)OC(C)(C)C)ccn2)CC1]

Intermediate 361 was prepared following the procedure used for the synthesis of Intermediate 189 starting from Intermediate 360 (250 mg, 0.63 mmol) and Intermediate 2 (233 mg, 0.69 mmol) to afford title compound (300 mg, 0.46 mmol, 74% yield). LC-MS (ESI): m/z (M+1): 645.4 (Method 4)

### Intermediate 362: N-[4-({3-[(2-aminoethyl)sulfanyl]-6-(5-chloro-2-fluorophenyl)pyridazin-4-yl}amino)pyridin-2-yl]-3-(4-methylpiperazin-1-yl)propanamide

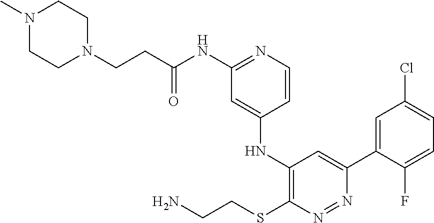 [SMILES: CN1CCN(CCC(=O)Nc2cc(Nc3cc(-c4cc(Cl)ccc4F)nnc3SCCN)ccn2)CC1]

Intermediate 362 was prepared following the procedure used for the synthesis of Intermediate 350 starting from Intermediate 361 (300 mg, 0.46 mmol) to afford title compound (250 mg, 0.45 mmol, 99% yield). LC-MS (ESI): m/z (M+1): 545.3 (Method 4)

### Intermediate 363: 6-(5-chloro-2-fluorophenyl)-N-{1H-pyrrolo[2,3-b]pyridin-4-yl}-3-{[2-(trimethylsilyl)ethyl]sulfanyl}pyridazin-4-amine

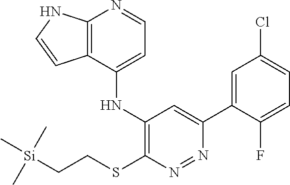 [SMILES: C[Si](C)(C)CCSc1nnc(-c2cc(Cl)ccc2F)cc1Nc1ccnc2[nH]ccc12]

A mixture of 4-chloro-7-azaindole (150 mg, 0.98 mmol), Pd(OAc)₂ (16 mg, 0.07 mmol), Xantphos (97 mg, 0.17 mmol), Cs₂CO₃ (458 mg, 1.4 mmol) and Intermediate 313 (250 mg, 0.70 mmol) in Toluene (6.1 mL) was degassed (vacuum/N₂) and then stirred at 115° C. for 28 hrs. The mixture was diluted with EtOAc and washed with saturate aqueous NaHCO₃ solution and brine. The organic phase was dried over Na₂SO₄, filtered and concentrated under reduced pressure. The crude material was purified by flash chromatography on Biotage silica cartridge (from cHex to 45% EtOAc), and then by reverse flash chromatography on Biotage C18 cartridge (from H₂O+0.1% HCOOH to 97% MeCN+0.1% HCOOH). Collected fractions were concentrated under reduced pressure, then dissolved in DCM and washed with sat. aq. NaHCO₃. The organic phase was dried over Na₂SO₄, filtered and concentrated under reduced pressure to afford title compound (140 mg, 0.30 mmol, 42% yield). LC-MS (ESI): m/z (M+1): 472.2 (Method 3)

### Intermediate 364 tert-butyl 4-{[(tert-butoxy)carbonyl][6-(5-chloro-2-fluorophenyl)-3-({[3-(methoxycarbonyl)phenyl]methyl}sulfanyl)pyridazin-4-yl]amino}-1H-pyrrolo[2,3-b]pyridine-1-carboxylate and Intermediate 365 tert-butyl 4-{[6-(5-chloro-2-fluorophenyl)-3-({[3-(methoxycarbonyl)phenyl]methyl}sulfanyl)pyridazin-4-yl]amino}-1H-pyrrolo[2,3-b]pyridine-1-carboxylate

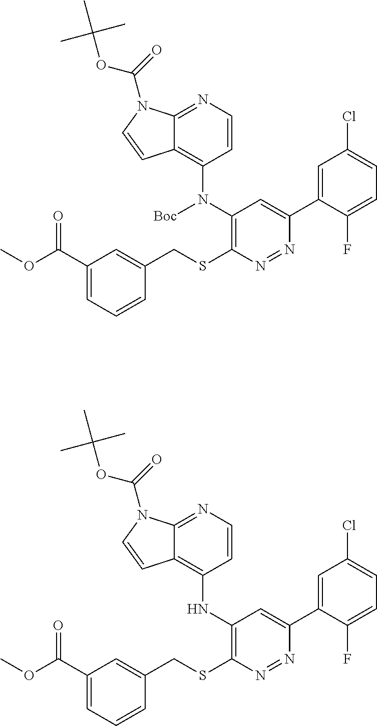 [SMILES: COC(=O)c1cccc(CSc2nnc(-c3cc(Cl)ccc3F)cc2N(C(=O)OC(C)(C)C)c2ccnc3c2ccn3C(=O)OC(C)(C)C)c1 ; COC(=O)c1cccc(CSc2nnc(-c3cc(Cl)ccc3F)cc2Nc2ccnc3c2ccn3C(=O)OC(C)(C)C)c1]

### Step 1

A solution of DMAP (3.6 mg, 0.03 mmol) and Intermediate 363 (140 mg, 0.30 mmol) in DCM (3 ml) was treated with a solution of di-tert-butyl dicarbonate (79 mg, 0.36 mmol) in DCM (0.50 mL). The mixture was stirred for 2 hrs at RT, then additional di-tert-butyl dicarbonate (155 mg, 0.71 mmol) was added and the mixture stirred for further 2 hrs. The reaction was diluted with DCM and washed with saturated aqueous NaHCO₃ solution. The organic phase was dried over Na₂SO₄, filtered, and concentrated under reduced pressure. The crude product was purified by flash chromatography on Biotage silica cartridge (from cHex to 20% EtOAc) to give tert-butyl 4-{[(tert-butoxy)carbonyl][6-(5-chloro-2-fluorophenyl)-3-{[2-(trimethylsilyl)ethyl]sulfanyl}pyridazin-4-yl]amino}-1H-pyrrolo[2,3-b]pyridine-1-carboxylate (195 mg, 0.29 mmol, 98% yield) as a mixture with its mono Boc derivative.

### Step 2

A solution of tert-butyl 4-{[(tert-butoxy)carbonyl][6-(5-chloro-2-fluorophenyl)-3-{[2-(trimethylsilyl)ethyl]sulfanyl}pyridazin-4-yl]amino}-1H-pyrrolo[2,3-b]pyridine-1-carboxylate (from Step 1, 195 mg, 0.29 mmol) in THE (1.9 mL) was treated with tetrabutylammonium fluoride 1M in THE (0.32 mL, 0.32 mmol) and stirred at RT for 24 hrs. A solution of methyl 3-(bromomethyl)benzoate (66 mg, 0.29 mmol) in THE (0.50 mL) was then added and the mixture stirred for 30 minutes. The reaction was diluted with EtOAcand washed with water. The organic phase was separated, dried over Na₂SO₄, filtered and concentrated under reduced pressure. The crude product was purified by flash chromatography on Biotage silica cartridge (from cHex to 25% EtOAc) to give tert-butyl 4-{[(tert-butoxy)carbonyl][6-(5-chloro-2-fluorophenyl)-3-({[3-(methoxycarbonyl)phenyl]methyl}sulfanyl)pyridazin-4-yl]amino}-1H-pyrrolo[2,3-b]pyridine-1-carboxylate (Intermediate 364, 84 mg, 0.12 mmol, 40% yield) and tert-butyl 4-{[6-(5-chloro-2-fluorophenyl)-3-({[3-(methoxycarbonyl)phenyl]methyl}sulfanyl)pyridazin-4-yl]amino}-1H-pyrrolo[2,3-b]pyridine-1-carboxylate (Intermediate 365, 72 mg, 0.12 mmol, 40% yield).

Intermediate 364: LC-MS (ESI): m/z (M+1): 720.4 (Method 4)

Intermediate 365: LC-MS (ESI): m/z (M+1): 620.3 (Method 4)

### Intermediate 366: 3-({[4-({1-[(tert-butoxy)carbonyl]-1H-pyrrolo[2,3-b]pyridin-4-yl}amino)-6-(5-chloro-2-fluorophenyl)pyridazin-3-yl]sulfanyl}methyl)benzoic acid

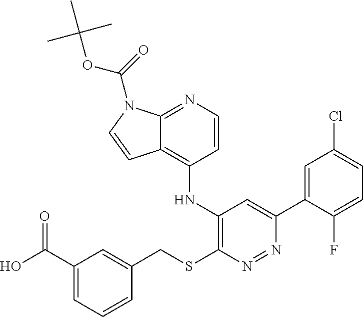 [SMILES: CC(C)(C)OC(=O)n1ccc2c(Nc3cc(-c4cc(Cl)ccc4F)nnc3SCc3cccc(C(=O)O)c3)ccnc21]

Lithium hydroxide hydrate (5 mg, 0.12 mmol) in H₂O (0.85 mL) was added to a stirred mixture of Intermediate 364 (84 mg, 0.12 mmol) in THE (2.8 mL). The reaction was stirred at RT overnight. Additional lithium hydroxide hydrate (10 mg, 0.23 mmol) in H₂O (0.84 mL) was added and the mixture stirred for 6 hrs. The mixture was concentrated under reduced pressure and the residue was purified by reverse flash chromatography on Biotage C18 cartridge (from H₂O to 50% MeCN) to afford title compound (36 mg, 0.06 mmol, 51% yield). LC-MS (ESI): m/z (M+1): 606.3 (Method 4)

### Intermediate 367: 3-({[6-(5-chloro-2-fluorophenyl)-4-({1H-pyrrolo[2,3-b]pyridin-4-yl}amino)pyridazin-3-yl]sulfanyl}methyl)benzoic acid

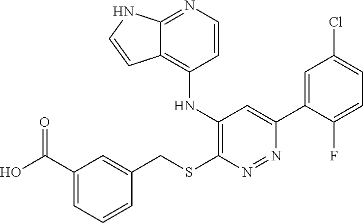 [SMILES: O=C(O)c1cccc(CSc2nnc(-c3cc(Cl)ccc3F)cc2Nc2ccnc3[nH]ccc23)c1]

Intermediate 367 was prepared following the procedure used for the synthesis of Intermediate 366 starting from Intermediate 365 (72 mg, 0.12 mmol) to afford title compound (40 mg, 0.08 mmol, 69% yield). LC-MS (ESI): m/z (M+1): 506.2 (Method 4)

### Intermediate 368: tert-butyl 4-{[6-(5-chloro-2-fluorophenyl)-3-{1[(3-{[(1-methylpiperidin-4-yl)methoxy]carbonyl}phenyl)methyl]sulfanyl}pyridazin-4-yl]amino}-1H-pyrrolo[2,3-b]pyridine-1-carboxylate

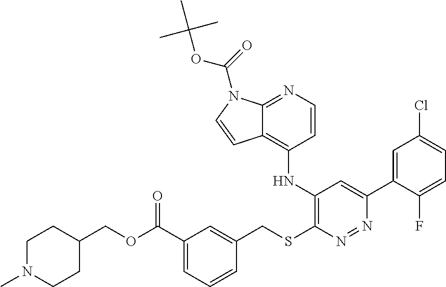 [SMILES: CN1CCC(COC(=O)c2cccc(CSc3nnc(-c4cc(Cl)ccc4F)cc3Nc3ccnc4c3ccn4C(=O)OC(C)(C)C)c2)CC1]

A solution of DIPEA (0.02 mL, 0.12 mmol), (1-methyl-4-piperidinyl)methanol (11 mg, 0.09 mmol) and Intermediate 366 (36 mg, 0.06 mmol) in DMF (0.6 ml) was treated with HATU (32 mg, 0.08 mmol). The mixture was stirred overnight at RT and then 4 hrs at 50° C. Additional (1-methyl-4-piperidinyl)methanol (15 mg, 0.12 mmol), DIPEA (0.03 mL, 0.18 mmol) and HATU (45 mg, 0.12 mmol) were added and the mixture stirred at 50° C. for 3 hrs. The mixture was diluted with EtOAc, washed with saturated aqueous NaHCO₃ solution and brine. The organic phase was separated, dried over Na₂SO₄, filtered and concentrated under reduced pressure. The crude product was purified by flash chromatography on Biotage silica NH cartridge (from cHex to 2% EtOAc/MeOH 10/1) afford title compound (30 mg, 0.04 mmol, 70% yield). LC-MS (ESI): m/z (M+1): 717.4 (Method 4)

### Intermediate 369: tert-butyl 4-(carbamoylmethyl)-1,4-diazepane-1-carboxylate

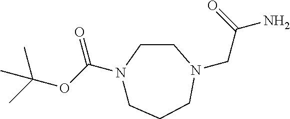 [SMILES: CC(C)(C)OC(=O)N1CCCN(CC(N)=O)CC1]

To a mixture of 1-Boc-hexahydro-1,4-diazepine (1.00 g, 4.99 mmol), 2-chloroacetamide (0.56 g, 5.99 mmol) and K₂CO₃ (0.75 g, 5.43 mmol), MeCN (30 mL) was added, and the resulting reaction mixture was stirred overnight at 70° C. The mixture was filtered, the solid was washed with MeCN, and the filtrate was concentrated under reduced pressure. The residue was treated with DCM and a saturated aqueous NaHCO₃ solution. The mixture was separated, the organic phase was washed with water, dried over Na₂SO₄, and the solvent removed under reduced pressure to afford title compound (1.04 g, 4.04 mmol, 81% yield).

### Intermediate 370: tert-butyl 4-{[(6-chloropyrimidin-4-yl)carbamoyl]methyl}-1,4-diazepane-1-carboxylate

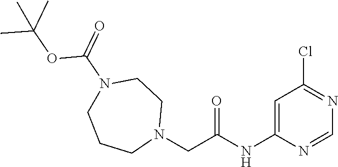 [SMILES: CC(C)(C)OC(=O)N1CCCN(CC(=O)Nc2cc(Cl)ncn2)CC1]

Intermediate 370 was prepared following the procedure used for the synthesis of Intermediate 272 starting from Intermediate 369 (432 mg, 1.68 mmol) and 4,6-dichloropyrimidine (263 mg, 1.76 mmol) to afford title compound (425 mg, 1.15 mmol, 68% yield). LC-MS (ESI): m/z (M+1): 370.8 (Method 4)

### Intermediate 371: tert-butyl 4-{[(6-{[6-(5-chloro-2-fluorophenyl)-3-{methyl[(3-methyl-2-oxooxolan-3-yl)methyl]amino}pyridazin-4-yl]amino}pyrimidin-4-yl)carbamoyl]methyl}-1,4-diazepane-1-carboxylate

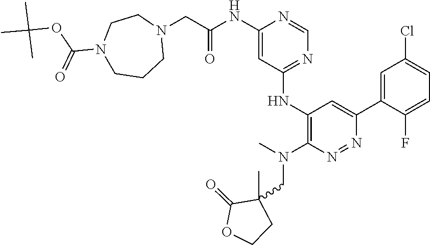 [SMILES: CN(CC1(C)CCOC1=O)c1nnc(-c2cc(Cl)ccc2F)cc1Nc1cc(NC(=O)CN2CCCN(C(=O)OC(C)(C)C)CC2)ncn1]

Intermediate 371 was prepared following the procedure used for the synthesis of Intermediate 189 starting from Intermediate 266 (244 mg, 0.67 mmol) and tert-butyl 4-{[(6-chloropyrimidin-4-yl)carbamoyl]methyl}-1,4-diazepane-1-carboxylate (Intermediate 370, 272 mg. 0.73 mmol) to afford title compound (291 mg, 0.42 mmol, 62% yield). LC-MS (ESI): m/z (M+1): 698.5 (Method 4)

### Intermediate 372: N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(1,4-diazepan-1-yl)acetamide

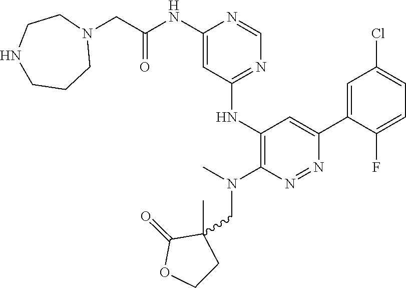 [SMILES: CN(CC1(C)CCOC1=O)c1nnc(-c2cc(Cl)ccc2F)cc1Nc1cc(NC(=O)CN2CCCNCC2)ncn1]

Intermediate 372 was prepared following the procedure used for the synthesis of Intermediate 40 starting from Intermediate 371 (291 mg, 0.42 mmol) to afford title compound (241 mg, 0.40 mmol, 97% yield mmol).

LC-MS (ESI): m/z (M+1): 598.4 (Method 3)

### PREPARATIONS OF EXAMPLES

### Example 1: N-(4-{[6-(5-chloro-2-fluorophenyl)-3-(2-hydroxyethoxy)pyridazin-4-yl]amino}pyridin-2-yl)-3-(4-methylpiperazin-1-yl)propanamide

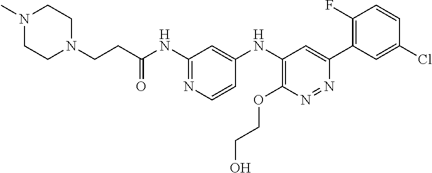 [SMILES: CN1CCN(CCC(=O)Nc2cc(Nc3cc(-c4cc(Cl)ccc4F)nnc3OCCO)ccn2)CC1]

Intermediate 2 (114 mg, 0.35 mmol) was added to a stirred mixture of Intermediate 4 (90 mg, 0.32 mmol), Pd(OAc)₂ (3.6 mg, 0.02 mmol), Xantphos (18.4 mg, 0.03 mmol) and Cs₂CO₃ (208 mg, 0.63 mmol) in dry 1,4-dioxane (3 mL) at RT. The mixture was degassed with N₂. The vial was closed, and the reaction was heated at 100° C. for 3 hrs. After cooling down the mixture was filtered over Celite® pad washing with 1,4-dioxane. The solvent was removed by reduced pressure, then it was purified by flash chromatography on Biotage silica cartridge (from DCM to 5% MeOH/0.5% H₂O) to afford the title compound (110 mg, 0.21 mmol, 65% yield).

LC-MS (ESI): m/z (M+1): 530.3 (Method 2)

¹H NMR (400 MHz, DMSO-d₆) δ ppm 10.67 (s, 1H), 8.88 (s, 1H), 8.19 (d, J=5.7 Hz, 1H), 8.13 (s, 1H), 7.92 (dd, J=6.5, 2.7 Hz, 1H), 7.67 (s, 1H), 7.58 (dt, J=8.7, 3.4 Hz, 1H), 7.41 (t, J=9.6 Hz, 1H), 7.07 (dd, J=5.6, 1.9 Hz, 1H), 4.99 (t, J=6.2 Hz, 1H), 4.55 (t, J=4.8 Hz, 2H), 3.86 (q, J=5.3 Hz, 2H), 2.51-2.66 (m, 4H), 2.16-2.48 (m, 8H), 2.14 (s, 3H).

### Example 2: 2-{[6-(5-chloro-2-fluorophenyl)-4-({7-[2-(4-methylpiperazin-1-yl)ethoxy]quinolin-4-yl}amino)pyridazin-3-yl]oxy}ethan-1-ol

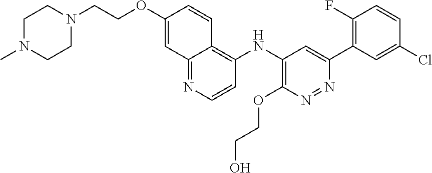 [SMILES: CN1CCN(CCOc2ccc3c(Nc4cc(-c5cc(Cl)ccc5F)nnc4OCCO)ccnc3c2)CC1]

Intermediate 5 (86 mg, 0.25 mmol) was added to a stirred mixture of 2-{[4-amino-6-(5-chloro-2-fluorophenyl)pyridazin-3-yl]oxy}ethan-1-ol (Intermediate 4, 60 mg, 0.21 mmol), Pd(OAc)₂ (2.4 mg, 0.01 mmol), Xantphos (12.2 mg, 0.02 mmol) and Cs₂CO₃ (139 mg, 0.42 mmol) in dry 1,4-dioxane (2 mL) at RT. The mixture was degassed with N₂, the vial was closed and the reaction was irradiated at 130° C. with MW for 2 hrs. After cooling down the mixture was filtered on Celite® pad washing with 1,4-dioxane. The solvent was removed by reduced pressure and the residue was purified by flash chromatography on Biotage silica cartridge (from DCM to 5% MeOH/0.5% H₂O) to afford the title compound (50 mg, 0.09 mmol, 43% yield).

LC-MS (ESI): m/z (M+1): 553.3 (Method 2)

¹H NMR (400 MHz, DMSO-d₆) δ ppm 8.43-9.17 (m, 2H), 8.01 (d, J=9.2 Hz, 1H), 7.87 (dd, J=6.6, 2.9 Hz, 1H), 7.54 (ddd, J=8.7, 4.1, 3.0 Hz, 1H), 7.19-7.47 (m, 5H), 5.05 (br s, 1H), 4.58 (t, J=4.7 Hz, 2H), 4.25 (t, J=5.7 Hz, 2H), 3.87 (br t, J=4.6 Hz, 2H), 2.76 (t, J=5.6 Hz, 2H), 2.45-2.61 (m, 4H), 2.23-2.42 (m, 4H), 2.15 (s, 3H).

### Example 3: N-[6-(5-chloro-2-fluorophenyl)-3-(2,2-difluoroethoxy)pyridazin-4-yl]-7-[2-(4-methylpiperazin-1-yl)ethoxy]quinolin-4-amine

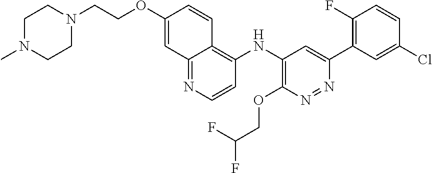 [SMILES: CN1CCN(CCOc2ccc3c(Nc4cc(-c5cc(Cl)ccc5F)nnc4OCC(F)F)ccnc3c2)CC1]

Example 3 was prepared following the procedure used for the synthesis of Example 2, starting from Intermediate 5 (66 mg, 0.22 mmol) and Intermediate 9 (60 mg, 0.20 mmol) to afford title compound (80 mg, 0.14 mmol, 71% yield).

LC-MS (ESI): m/z (M+1): 573.3 (Method 2)

¹H NMR (500 MHz, Chloroform-d) δ ppm 8.82 (d, J=4.9 Hz, 1H), 8.06 (dd, J=6.6, 2.7 Hz, 1H), 7.83 (d, J=9.3 Hz, 1H), 7.65 (d, J=1.5 Hz, 1H), 7.48 (d, J=2.5 Hz, 1H), 7.38 (ddd, J=8.7, 4.2, 2.8 Hz, 1H), 7.34 (d, J=4.9 Hz, 1H), 7.31 (dd, J=9.2, 2.6 Hz, 1H), 7.16 (s, 1H), 7.11 (dd, J=10.4, 8.8 Hz, 1H), 6.38 (tt, J=55.1, 3.9 Hz, 1H), 4.96 (td, J=13.4, 3.9 Hz, 2H), 4.30 (t, J=5.6 Hz, 2H), 2.93 (t, J=5.7 Hz, 2H), 2.37-2.83 (m, 8H), 2.32 (s, 3H).

### Example 4: N-(4-{[6-(5-chloro-2-fluorophenyl)-3-[3-(methylsulfanyl)propoxy]pyridazin-4-yl]amino}pyridin-2-yl)-3-(4-methylpiperazin-1-yl)propanamide

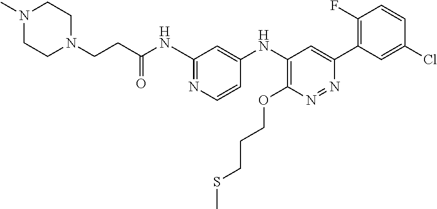 [SMILES: CSCCCOc1nnc(-c2cc(Cl)ccc2F)cc1Nc1ccnc(NC(=O)CCN2CCN(C)CC2)c1]

Example 4 was prepared following the procedure used for the synthesis of Example 1, starting from Intermediate 12 (24 mg, 0.07 mmol) and Intermediate 2 (26 mg, 0.08 mmol) to afford title compound (20 mg, 0.03 mmol, 48% yield).

LC-MS (ESI): m/z (M+1): 574.5 (Method 2)

¹H NMR (400 MHz, Chloroform-d) δ ppm 11.20 (s, 1H), 8.24 (d, J=5.5 Hz, 1H), 8.06-8.13 (m, 2H), 7.77 (d, J=1.6 Hz, 1H), 7.37 (ddd, J=8.8, 4.3, 2.8 Hz, 1H), 7.13 (dd, J=10.6, 8.8 Hz, 1H), 7.09 (s, 1H), 6.92-6.99 (m, 1H), 4.79 (t, J=6.3 Hz, 2H), 2.73-2.81 (m, 4H), 2.53-2.59 (m, 2H), 2.45-2.72 (m, 8H), 2.37 (s, 3H), 2.27 (quin, J=6.6 Hz, 2H), 2.20 (s, 3H).

### Example 5: N-[3-(2-aminoethoxy)-6-(5-chloro-2-fluorophenyl)pyridazin-4-yl]-7-[2-(4-methylpiperazin-1-yl)ethoxy]quinolin-4-amine

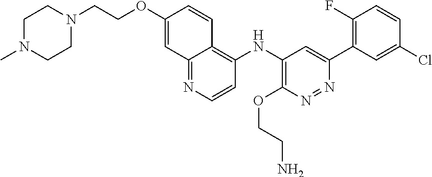 [SMILES: CN1CCN(CCOc2ccc3c(Nc4cc(-c5cc(Cl)ccc5F)nnc4OCCN)ccnc3c2)CC1]

Intermediate 18 (150 mg, 0.21 mmol) and methylamine 33% in EtOH (9.1 mL, 72.4 mmol) were mixed and stirred at RT for 2 hrs. Volatiles were removed under reduced pressure, the residue was treated with water and 2N HCl, then extracted with EtOAc. Aqueous phase was treated with 33% NH₄OH until pH 10 and extracted with DCM. Organic layer was separated, dried over Na₂SO₄ and evaporated to afford the title compound (80 mg, 0.14 mmol, 69% yield).

LC-MS (ESI): m/z (M+1): 552.3 (Method 2)

¹H NMR (500 MHz, DMSO-d₆) δ ppm 8.57-8.85 (m, 1H), 7.99 (br d, J=9.1 Hz, 1H), 7.87 (dd, J=6.6, 2.7 Hz, 1H), 7.51-7.58 (m, 1H), 7.22-7.49 (m, 4H), 7.20 (d, J=1.4 Hz, 1H), 4.50 (br t, J=5.2 Hz, 2H), 4.25 (t, J=5.6 Hz, 2H), 2.99 (br t, J=4.8 Hz, 2H), 2.76 (t, J=5.8 Hz, 2H), 2.47-2.61 (m, 4H), 2.21-2.45 (m, 4H), 2.15 (s, 3H).

### Example 6: N-(2-{[6-(5-chloro-2-fluorophenyl)-4-({7-[2-(4-methylpiperazin-1-yl)ethoxy]quinolin-4-yl}amino)pyridazin-3-yl]oxy}ethyl)methanesulfonamide

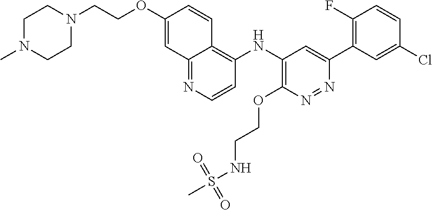 [SMILES: CN1CCN(CCOc2ccc3c(Nc4cc(-c5cc(Cl)ccc5F)nnc4OCCNS(C)(=O)=O)ccnc3c2)CC1]

Methanesulfonyl chloride (5 μL, 0.06 mmol) was added to a stirred solution of Example 5 (30 mg, 0.05 mmol) and TEA (10 μL, 0.11 mmol) in DCM (4 mL) at RT. After 1 h, the solvent was removed under reduced pressure. The residue was purified by flash chromatography on Biotage silica NH cartridge (from DCM to 5% MeOH/0.5% H₂O) to afford the title compound (20 mg, 0.03 mmol, 59% yield).

LC-MS (ESI): m/z (M+1): 630.3 (Method 2)

¹H NMR (500 MHz, DMSO-d₆) δ ppm 8.68-8.92 (m, 2H), 7.94-8.11 (m, 1H), 7.82-7.91 (m, 1H), 7.51-7.57 (m, 1H), 7.25-7.52 (m, 5H), 7.23-7.27 (m, 1H), 4.55-4.79 (m, 2H), 4.14-4.33 (m, 2H), 3.53 (br s, 2H), 3.00 (s, 3H), 2.76 (t, J=5.7 Hz, 2H), 2.22-2.62 (m, 8H), 2.15 (s, 3H).

### Example 7: N-(4-{[6-(5-chloro-2-fluorophenyl)-3-(3-methanesulfonylpropoxy)pyridazin-4-yl]amino}pyridin-2-yl)-3-(4-methylpiperazin-1-yl)propanamide

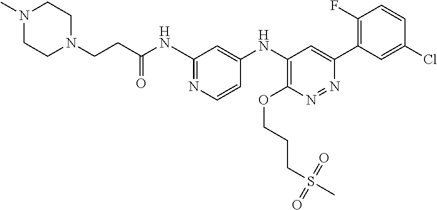 [SMILES: CN1CCN(CCC(=O)Nc2cc(Nc3cc(-c4cc(Cl)ccc4F)nnc3OCCCS(C)(=O)=O)ccn2)CC1]

Example 7 was prepared following the procedure used for the synthesis of Example 1, starting from Intermediate 21 (30 mg, 0.08 mmol) and Intermediate 2 (30 mg, 0.09 mmol) to afford title compound (18 mg, 0.03 mmol, 36% yield).

LC-MS (ESI): m/z (M+1): 606.3 (Method 2)

¹H NMR (500 MHz, DMSO-d₆) δ ppm 10.66 (s, 1H), 8.95 (s, 1H), 8.18 (d, J=5.6 Hz, 1H), 8.13 (d, J=1.1 Hz, 1H), 7.93 (dd, J=6.6, 2.9 Hz, 1H), 7.67 (s, 1H), 7.55-7.62 (m, 1H), 7.41 (dd, J=10.4, 8.8 Hz, 1H), 7.05 (dd, J=5.6, 2.1 Hz, 1H), 4.66 (t, J=6.2 Hz, 2H), 3.40-3.49 (m, 2H), 3.03 (s, 3H), 2.57-2.63 (m, 2H), 2.51-2.55 (m, 2H), 2.33 (br s, 10H), 2.14 (s, 3H).

### Example 8: N-(4-{[3-(2-aminoethoxy)-6-(5-chloro-2-fluorophenyl)pyridazin-4-yl]amino}pyridin-2-yl)-3-(4-methylpiperazin-1-yl)propanamide

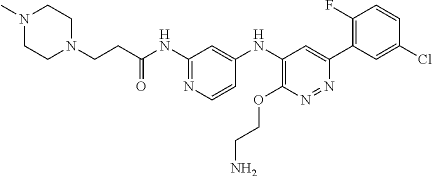 [SMILES: CN1CCN(CCC(=O)Nc2cc(Nc3cc(-c4cc(Cl)ccc4F)nnc3OCCN)ccn2)CC1]

Example 8 was prepared following the procedure used for the synthesis of Example 5, starting from Intermediate 22 (110 mg, 0.16 mmol) to afford title compound (50 mg, 0.09 mmol, 59% yield).

LC-MS (ESI): m/z (M+1): 529.3 (Method 2)

¹H NMR (500 MHz, DMSO-d₆) δ ppm 10.66 (s, 1H), 8.18 (d, J=5.6 Hz, 1H), 8.13 (d, J=1.1 Hz, 1H), 7.92 (dd, J=6.6, 2.7 Hz, 1H), 7.66 (d, J=1.0 Hz, 1H), 7.58 (ddd, J=8.8, 4.1, 2.7 Hz, 1H), 7.41 (dd, J=10.3, 8.9 Hz, 1H), 7.31-7.45 (m, 1H), 7.06 (dd, J=5.7, 2.1 Hz, 1H), 4.47 (t, J=5.4 Hz, 2H), 2.99 (t, J=5.4 Hz, 2H), 2.61 (t, J=6.3 Hz, 2H), 2.51-2.55 (m, 2H), 2.17-2.57 (m, 8H), 2.14 (s, 3H).

### Example 9: N-(4-{[6-(5-chloro-2-fluorophenyl)-3-(2-methanesulfonamidoethoxy)pyridazin-4-yl]amino}pyridin-2-yl)-3-(4-methylpiperazin-1-yl)propanamide

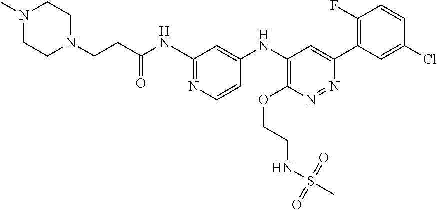 [SMILES: CN1CCN(CCC(=O)Nc2cc(Nc3cc(-c4cc(Cl)ccc4F)nnc3OCCNS(C)(=O)=O)ccn2)CC1]

Example 9 was prepared following the procedure used for the synthesis of Example 6, starting from Example 8 (20 mg, 0.04 mmol) to afford title compound (19 mg, 0.03 mmol, 83% yield).

LC-MS (ESI): m/z (M+1): 607.3 (Method 2)

¹H NMR (400 MHz, DMSO-d₆) δ ppm 10.68 (s, 1H), 8.84 (s, 1H), 8.19 (d, J=5.7 Hz, 1H), 8.13 (d, J=1.5 Hz, 1H), 7.92 (dd, J=6.6, 2.9 Hz, 1H), 7.69 (d, J=1.1 Hz, 1H), 7.54-7.63 (m, 1H), 7.29-7.51 (m, 2H), 7.06 (dd, J=5.6, 2.1 Hz, 1H), 4.61 (t, J=5.4 Hz, 2H), 3.46-3.65 (m, 2H), 3.00 (s, 3H), 2.57-2.66 (m, 2H), 2.51-2.56 (m, 2H), 2.18-2.56 (m, 8H), 2.14 (s, 3H).

### Example 10: methyl 4-{[6-(5-chloro-2-fluorophenyl)-3-[3-(dimethylamino)propoxy]pyridazin-4-yl]amino}-1H-pyrrolo[2,3-b]pyridine-2-carboxylate

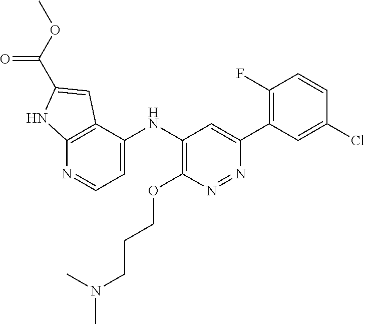 [SMILES: COC(=O)c1cc2c(Nc3cc(-c4cc(Cl)ccc4F)nnc3OCCCN(C)C)ccnc2[nH]1]

TFA (0.01 mL, 0.11 mmol) was added to a stirred solution of Intermediate 27 (70 mg, 0.11 mmol) in DCM (5 mL). After 2 hrs at RT, volatiles were removed under vacuum and the residue was charged on SCX, washed with MeOH and eluted with 1N NH₃ in MeOH. Evaporation of basic fractions afforded the title compound (44 mg, 0.09 mmol, 79% yield).

LC-MS (ESI): m/z (M+1): 499.4 (Method 2)

¹H NMR (400 MHz, DMSO-d₆) δ ppm 12.52 (s, 1H), 8.95 (br s, 1H), 8.29 (d, J=5.4 Hz, 1H), 7.89 (dd, J=6.6, 2.8 Hz, 1H), 7.51-7.61 (m, 2H), 7.39 (dd, J=10.5, 8.9 Hz, 1H), 7.27 (d, J=1.4 Hz, 1H), 7.02 (d, J=5.4 Hz, 1H), 4.61 (t, J=6.6 Hz, 2H), 3.86 (s, 3H), 2.39 (t, J=7.0 Hz, 2H), 2.15 (s, 6H), 1.99 (quin, J=6.8 Hz, 2H).

### Example 11: (3-{[6-(5-chloro-2-fluorophenyl)-4-{[2-(methoxycarbonyl)-1H-pyrrolo[2,3-b]pyridin-4-yl]amino}pyridazin-3-yl]oxy}propyl)trimethylazanium chloride

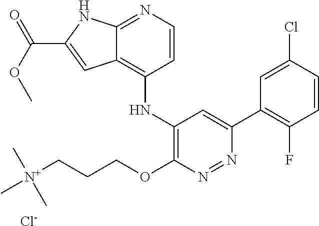 [SMILES: COC(=O)c1cc2c(Nc3cc(-c4cc(Cl)ccc4F)nnc3OCCC[N+](C)(C)C)ccnc2[nH]1 ; [Cl-]]

Iodomethane (4.49 μL, 0.07 mmol) was added to a suspension of Example 10 (36 mg, 0.07 mmol) in MeCN (1.5 mL) and MeOH (1.5 mL). The mixture was stirred at 45° C. for 1 h. Volatiles were removed under vacuum and the residue was purified by reverse flash chromatography on Biotage C18 cartridge (from H₂O+0.1% HCl to 45% MeCN). Evaporation of opportune fractions afforded the title compound (36 mg, 0.07 mmol, 92% yield).

LC-MS (ESI): m/z (M+1) 513.3 (Method 1)

¹H NMR (400 MHz, DMSO-d₆) δ ppm 12.93-13.39 (m, 1H), 10.14-11.34 (m, 1H), 8.32 (d, J=6.0 Hz, 1H), 7.87 (dd, J=6.4, 2.7 Hz, 1H), 7.69-7.82 (m, 1H), 7.52-7.68 (m, 2H), 7.46 (br t, J=9.6 Hz, 1H), 7.03-7.20 (m, 1H), 4.67 (br t, J=5.7 Hz, 2H), 3.90 (s, 3H), 3.60-3.79 (m, 2H), 3.11 (s, 9H), 2.23-2.37 (m, 2H).

### Example 12: methyl 4-{[(4-{[6-(5-chloro-2-fluorophenyl)-3-[2-(dimethylamino)ethoxy]pyridazin-4-yl]amino}pyridin-2-yl)carbamoyl]methyl}-1-methylpiperazine-2-carboxylate

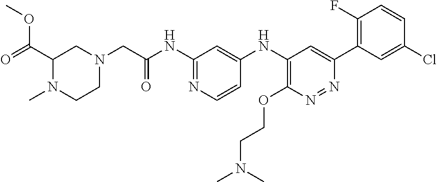 [SMILES: COC(=O)C1CN(CC(=O)Nc2cc(Nc3cc(-c4cc(Cl)ccc4F)nnc3OCCN(C)C)ccn2)CCN1C]

Example 12 was prepared following the procedure used for the synthesis of Example 1, starting from Intermediate 30 (100 mg, 0.30 mmol) and Intermediate 34 (121 mg, 0.33 mmol) to afford title compound (55 mg, 0.09 mmol, 31% yield).

LC-MS (ESI): m/z (M+1): 601.2 (Method 2)

¹H NMR (400 MHz, DMSO-d₆) δ ppm 9.75 (s, 1H), 9.06 (s, 1H), 8.17 (d, J=5.7 Hz, 1H), 8.10 (d, J=1.8 Hz, 1H), 7.92 (dd, J=6.6, 2.9 Hz, 1H), 7.69 (d, J=1.3 Hz, 1H), 7.54-7.61 (m, 1H), 7.42 (dd, J=10.4, 8.9 Hz, 1H), 7.08 (dd, J=5.7, 2.2 Hz, 1H), 4.66 (t, J=6.1 Hz, 2H), 3.63 (s, 3H), 3.07-3.27 (m, 3H), 2.87-3.02 (m, 1H), 2.71-2.84 (m, 3H), 2.53-2.69 (m, 3H), 2.29-2.38 (m, 1H), 2.21-2.29 (m, 9H).

### Example 13: N-(4-{[6-(5-chloro-2-fluorophenyl)-3-methoxypyridazin-4-yl]amino}pyridin-2-yl)-3-[4-(2,2,2-trifluoroethyl)piperazin-1-yl]propanamide

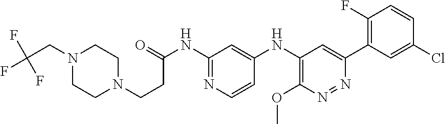 [SMILES: COc1nnc(-c2cc(Cl)ccc2F)cc1Nc1ccnc(NC(=O)CCN2CCN(CC(F)(F)F)CC2)c1]

Example 13 was prepared following the procedure used for the synthesis of Example 1, starting from 6-(5-chloro-2-fluorophenyl)-3-methoxypyridazin-4-amine (Intermediate 37, 50 mg, 0.20 mmol) and Intermediate 38 (86 mg, 0.22 mmol) to afford title compound (90 mg, 0.16 mmol, 80% yield).

LC-MS (ESI): m/z (M+1): 568.2 (Method 2)

¹H NMR (400 MHz, Chloroform-d) δ ppm 11.13 (s, 1H), 8.22 (d, J=5.3 Hz, 1H), 8.11 (dd, J=6.6, 2.6 Hz, 1H), 8.07 (d, J=1.8 Hz, 1H), 7.77 (s, 1H), 7.34-7.41 (m, 1H), 7.13 (dd, J=10.5, 8.8 Hz, 1H), 6.92-6.98 (m, 2H), 4.30 (s, 3H), 3.04 (q, J=9.6 Hz, 2H), 2.86 (br t, J=4.4 Hz, 4H), 2.73-2.81 (m, 2H), 2.62-2.73 (m, 4H), 2.51-2.61 (m, 2H).

### Example 14: N-(4-{[6-(5-chloro-2-fluorophenyl)-3-methoxypyridazin-4-yl]amino}pyridin-2-yl)-2-[4-(2,2,2-trifluoroethyl)piperazin-1-yl]acetamide

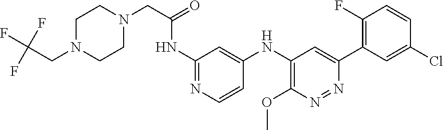 [SMILES: COc1nnc(-c2cc(Cl)ccc2F)cc1Nc1ccnc(NC(=O)CN2CCN(CC(F)(F)F)CC2)c1]

Example 14 was prepared following the procedure used for the synthesis of Example 1, starting from Intermediate 37 (43 mg, 0.17 mmol) and Intermediate 41 (70 mg, 0.18 mmol) to afford title compound (56 mg, 0.10 mmol, 60% yield).

LC-MS (ESI): m/z (M+1): 554.1 (Method 2)

¹H NMR (500 MHz, Chloroform-d) δ ppm 9.57 (s, 1H), 8.26 (d, J=5.6 Hz, 1H), 8.13 (d, J=2.1 Hz, 1H), 8.12 (dd, J=6.8, 2.8 Hz, 1H), 7.78 (d, J=1.4 Hz, 1H), 7.38 (ddd, J=8.7, 4.2, 2.8 Hz, 1H), 7.14 (dd, J=10.6, 8.8 Hz, 1H), 7.00 (dd, J=5.6, 2.2 Hz, 1H), 6.97 (s, 1H), 4.30 (s, 3H), 3.19 (s, 2H), 2.95-3.08 (m, 2H), 2.76-2.86 (m, 4H), 2.62-2.75 (m, 4H).

### Example 15: methyl 2-{[6-(5-chloro-2-fluorophenyl)-4-({2-[3-(4-methylpiperazin-1-yl)propanamido]pyridin-4-yl}amino)pyridazin-3-yl]oxy}acetate

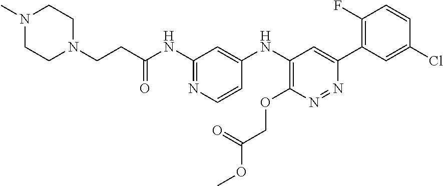 [SMILES: COC(=O)COc1nnc(-c2cc(Cl)ccc2F)cc1Nc1ccnc(NC(=O)CCN2CCN(C)CC2)c1]

A solution of Intermediate 47 (42 mg, 0.07 mmol) in 1.25 M HCl in MeOH (1.0 mL, 1.25 mmol) was shaken at RT for 30 minutes. Volatiles were removed at reduced pressure (keeping the rotavapor bath at 30° C.) then the residue was dissolved in MeOH and stirred overnight at 45° C. Volatiles were removed at reduced pressure and the crude the crude was purified by flash chromatography on Biotage silica NH cartridge (from DCM to 1% MeOH). Proper fractions were collected and purified by preparative HPLC, to afford the title compound (4 mg, 0.01 mmol, 11% yield).

LC-MS (ESI): m/z (M+1): 558.2 (Method 2)

¹H NMR (400 MHz, Chloroform-d) δ ppm 11.23 (s, 1H), 8.25 (d, J=5.6 Hz, 1H), 8.06-8.13 (m, 2H), 7.82 (s, 1H), 7.33-7.42 (m, 1H), 7.13 (dd, J=10.4, 8.9 Hz, 1H), 7.05 (s, 1H), 6.95 (dd, J=5.6, 2.0 Hz, 1H), 5.25 (s, 2H), 3.84 (s, 3H), 2.74-2.79 (m, 2H), 2.54-2.59 (m, 2H), 2.63 (br s, 8H), 2.37 (s, 3H).

### Example 16: N-(4-{[6-(5-chloro-2-fluorophenyl)-3-(methylsulfanyl)pyridazin-4-yl]amino}pyridin-2-yl)-3-(4-methylpiperazin-1-yl)propanamide

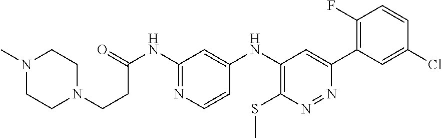 [SMILES: CSc1nnc(-c2cc(Cl)ccc2F)cc1Nc1ccnc(NC(=O)CCN2CCN(C)CC2)c1]

Example 16 was prepared following the procedure used for the synthesis of Example 1, starting from Intermediate 50 (50 mg, 0.18 mmol) and Intermediate 2 (65 mg, 0.20 mmol) to afford title compound (25 mg, 0.05 mmol, 27% yield).

LC-MS (ESI): m/z (M+1): 516.3 (Method 2)

¹H NMR (600 MHz, Chloroform-d) δ ppm 11.18 (br s, 1H), 8.23 (d, J=5.6 Hz, 1H), 8.20 (dd, J=6.7, 2.7 Hz, 1H), 8.04 (d, J=2.0 Hz, 1H), 7.72 (d, J=1.2 Hz, 1H), 7.38 (ddd, J=8.7, 4.3, 2.8 Hz, 1H), 7.13 (dd, J=10.6, 8.8 Hz, 1H), 6.91 (dd, J=5.7, 2.2 Hz, 1 H), 6.32 (s, 1H), 2.88 (s, 3H), 2.75-2.80 (m, 2H), 2.54-2.58 (m, 2H), 2.45-2.84 (m, 8H), 2.38 (s, 3H).

### Example 17: N-(4-{[6-(5-chloro-2-fluorophenyl)-3-methanesulfinylpyridazin-4-yl]amino}pyridin-2-yl)-3-(4-methylpiperazin-1-yl)propanamide

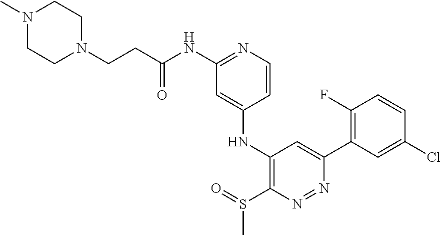 [SMILES: CN1CCN(CCC(=O)Nc2cc(Nc3cc(-c4cc(Cl)ccc4F)nnc3S(C)=O)ccn2)CC1]

A solution of Intermediate 51 (19 mg, 0.04 mmol) in DCM (0.3 mL) was treated with 1-methylpiperazine (13 mg, 0.13 mmol) and stirred at RT for 16 hrs. The mixture was concentrated and the residue purified by flash chromatography on Biotage silica NH cartridge (from c-Hex to 100% EtOAc) to afford the title compound (9 mg, 0.02 mmol, 38% yield).

LC-MS (ESI): m/z (M+1): 532.3 (Method 2)

¹H NMR (500 MHz, Chloroform-d) δ ppm 11.15-11.36 (m, 1H), 9.98 (s, 1H), 8.24 (d, J=5.6 Hz, 1H), 8.20 (d, J=1.9 Hz, 1H), 8.13 (dd, J=6.5, 2.7 Hz, 1H), 7.92 (d, J=0.7 Hz, 1H), 7.37-7.46 (m, 1H), 7.15 (dd, J=10.5, 8.9 Hz, 1H), 6.82 (dd, J=5.6, 2.1 Hz, 1H), 3.21 (s, 3H), 2.39-2.92 (m, 12H), 2.36 (s, 3H).

### Example 18: N-(4-{[6-(5-chloro-2-fluorophenyl)-3-methanesulfonylpyridazin-4-yl]amino}pyridin-2-yl)-3-(4-methylpiperazin-1-yl)propanamide

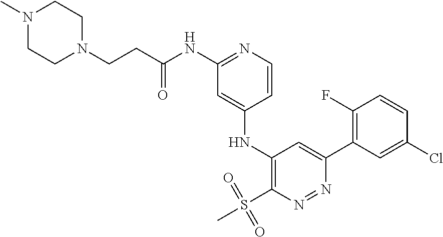 [SMILES: CN1CCN(CCC(=O)Nc2cc(Nc3cc(-c4cc(Cl)ccc4F)nnc3S(C)(=O)=O)ccn2)CC1]

Example 18 was prepared following the procedure used for the synthesis of Example 17, starting from Intermediate 52 (33 mg, 0.07 mmol) and 1-methylpiperazine (22 mg, 0.22 mmol) to afford title compound (9 mg, 0.02 mmol, 22% yield).

LC-MS (ESI): m/z (M+1): 548.3 (Method 2)

¹H NMR (500 MHz, Chloroform-d) δ ppm 11.37 (br s, 1H), 8.77 (s, 1H), 8.30 (d, J=5.5 Hz, 1H), 8.16-8.23 (m, 2H), 7.97 (s, 1H), 7.45 (ddd, J=8.8, 4.1, 2.7 Hz, 1H), 7.16 (dd, J=10.5, 8.9 Hz, 1H), 6.89 (dd, J=5.6, 2.1 Hz, 1H), 3.59 (s, 3H), 2.75-2.78 (m, 2H), 2.54-2.58 (m, 2H), 2.44-2.91 (m, 8H), 2.38 (s, 3H).

### Example 19: N-(4-{[6-(5-chloro-2-fluorophenyl)-3-[imino(methyl)oxo-λ⁶-sulfanyl]pyridazin-4-yl]amino}pyridin-2-yl)-3-(4-methylpiperazin-1-yl)propanamide

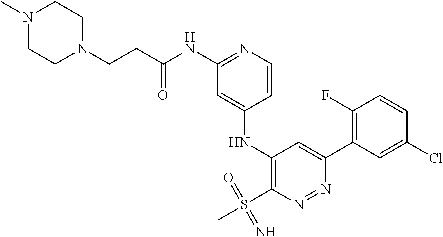 [SMILES: CN1CCN(CCC(=O)Nc2cc(Nc3cc(-c4cc(Cl)ccc4F)nnc3S(C)(=N)=O)ccn2)CC1]

TFA (0.1 mL, 1.3 mmol) was added to a stirred solution of Intermediate 56 (28 mg, 0.04 mmol) in DCM (0.4 mL). The mixture was stirred at RT for 1 h. Volatiles were removed under vacuum, the residue was charged in a SCX cartridge, washing with MeOH, and eluting with 1 N NH₃ in MeOH. Basic fractions were collected and evaporated and the obtained residue was purified by flash chromatography on Biotage silica NH cartridge (from c-Hex to 100% EtOAc), then it was further purified by flash chromatography on Biotage silica NH cartridge (from DCM to 3% MeOH) to afford the title compound (11 mg, 0.02 mmol, 47% yield).

LC-MS (ESI): m/z (M+1): 547.4 (Method 2)

¹H NMR (400 MHz, Chloroform-d) δ ppm 11.28 (s, 1H), 10.11 (br s, 1H), 8.28 (d, J=5.5 Hz, 1H), 8.20 (dd, J=6.6, 2.6 Hz, 1H), 8.16 (d, J=1.9 Hz, 1H), 7.97 (s, 1H), 7.44 (ddd, J=8.7, 4.1, 2.9 Hz, 1H), 7.16 (dd, J=10.5, 8.8 Hz, 1H), 6.89 (dd, J=5.5, 2.0 Hz, 1H), 3.61 (s, 4H), 2.73-2.81 (m, 2H), 2.52-2.59 (m, 2H), 2.44-2.93 (m, 8H), 2.38 (s, 3H).

### Example 20: 3-[4-(2-aminoethyl)piperazin-1-yl]-N-(4-{[6-(5-chloro-2-fluorophenyl)-3-(methylsulfanyl)pyridazin-4-yl]amino}pyridin-2-yl)propanamide

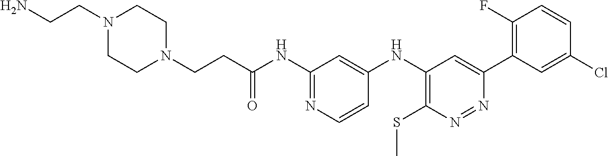 [SMILES: CSc1nnc(-c2cc(Cl)ccc2F)cc1Nc1ccnc(NC(=O)CCN2CCN(CCN)CC2)c1]

Example 20 was prepared following the procedure used for the synthesis of Example 19, starting from Intermediate 60 (110 mg, 0.13 mmol) to afford title compound (13 mg, 0.02 mmol, 19% yield).

LC-MS (ESI): m/z (M+1): 545.5 (Method 2)

¹H NMR (500 MHz, Chloroform-d) δ ppm 11.28 (s, 1H), 8.22 (d, J=5.6 Hz, 1H), 8.20 (dd, J=6.7, 2.7 Hz, 1H), 8.04 (d, J=2.1 Hz, 1H), 7.72 (d, J=1.4 Hz, 1H), 7.33-7.44 (m, 1H), 7.13 (dd, J=10.6, 8.8 Hz, 1H), 6.91 (dd, J=5.6, 2.1 Hz, 1H), 6.31 (s, 1H), 2.88 (s, 3H), 2.83 (t, J=6.2 Hz, 2H), 2.73-2.79 (m, 2H), 2.57-2.74 (m, 8H), 2.54-2.58 (m, 2H), 2.52 (t, J=6.2 Hz, 2H).

### Example 21: methyl N-[2-(4-{2-[(4-{[6-(5-chloro-2-fluorophenyl)-3-(methylsulfanyl)pyridazin-4-yl]amino}pyridin-2-yl)carbamoyl]ethyl}piperazin-1-yl)ethyl]carbamate

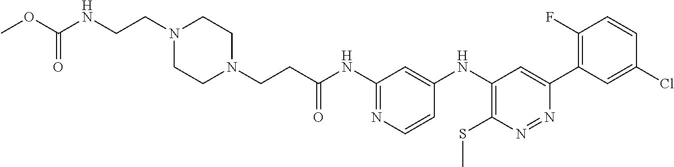 [SMILES: COC(=O)NCCN1CCN(CCC(=O)Nc2cc(Nc3cc(-c4cc(Cl)ccc4F)nnc3SC)ccn2)CC1]

To an ice-cooled solution of Example 20 (44 mg, 0.08 mmol) and TEA (23 μL, 0.16 mmol) in dry DCM (0.6 mL), methyl chloroformate (6.86 μL, 0.09 mmol) was added and the mixture was allowed to reach the RT and stirred for 30 min. The mixture was diluted with DCM and washed with saturated NaHCO₃ aqueous solution (2×). The combined organic layers were filtered through a phase separator and concentrated under vacuum. The crude material was purified by flash chromatography on Biotage silica NH cartridge (from DCM to 1% MeOH) affording the title compound (26 mg, 0.04 mmol, 53% yield).

LC-MS (ESI): m/z (M+1): 603.4 (Method 2)

¹H NMR (500 MHz, Chloroform-d) δ ppm 11.22 (s, 1H), 8.22 (d, J=5.6 Hz, 1H), 8.20 (dd, J=6.7, 2.7 Hz, 1H), 8.03 (d, J=2.1 Hz, 1H), 7.72 (d, J=1.1 Hz, 1H), 7.38 (ddd, J=8.8, 4.3, 2.7 Hz, 1H), 7.13 (dd, J=10.6, 8.8 Hz, 1H), 6.91 (dd, J=5.7, 2.1 Hz, 1H), 6.31 (s, 1H), 5.18 (br s, 1H), 3.69 (s, 3H), 3.18-3.43 (m, 2H), 2.88 (s, 3H), 2.73-2.80 (m, 2H), 2.58-2.72 (m, 8H), 2.52-2.59 (m, 4H).

### Example 22: N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)propanamide

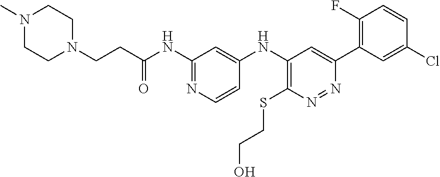 [SMILES: CN1CCN(CCC(=O)Nc2cc(Nc3cc(-c4cc(Cl)ccc4F)nnc3SCCO)ccn2)CC1]

To a solution of Intermediate 68 (134 mg, 0.20 mmol) in THE (2.5 mL), tetrabutylammonium fluoride 1M in THE (0.22 mL, 0.22 mmol) was added and the mixture was stirred at RT for 3 hrs. Volatiles were evaporated at reduced pressure and the crude material was purified by flash chromatography on Biotage silica NH cartridge (from DCM to 2% MeOH). Proper fractions were evaporated and further purified by preparative HPLC to give the title compound (65 mg, 0.12 mmol, 59% yield).

LC-MS (ESI): m/z (M+1): 546.2 (Method 2)

¹H NMR (400 MHz, Chloroform-d) δ ppm 11.27 (s, 1H), 8.24 (d, J=5.7 Hz, 1H), 8.14 (dd, J=6.7, 2.7 Hz, 1H), 8.05 (d, J=1.9 Hz, 1H), 7.73 (d, J=0.8 Hz, 1H), 7.39 (ddd, J=8.7, 4.1, 2.8 Hz, 1H), 7.13 (dd, J=10.5, 8.9 Hz, 1H), 6.91 (dd, J=5.6, 2.1 Hz, 1H), 6.51 (s, 1H), 4.07 (t, J=5.5 Hz, 2H), 3.66 (t, J=5.5 Hz, 2H), 3.10-3.59 (m, 1H), 2.41-3.08 (m, 12H), 2.37 (s, 3H).

### Example 23: N-(4-{[6-(5-chloro-2-fluorophenyl)-3-[(2-hydroxyethyl)sulfanyl]pyridazin-4-yl]amino}pyridin-2-yl)-4-(4-methylpiperazin-1-yl)butanamide

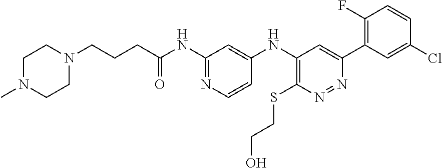 [SMILES: CN1CCN(CCCC(=O)Nc2cc(Nc3cc(-c4cc(Cl)ccc4F)nnc3SCCO)ccn2)CC1]

To a solution of Intermediate 71 (98 mg, 0.15 mmol) in DCM (2.9 mL), TFA (0.11 mL, 1.45 mmol) was added. The reaction was stirred at RT for 2 hrs. Volatiles were removed under vacuum. The residue was loaded on SCX (2 g, washing with MeOH, and eluting with 1N NH₃ in MeOH). Basic fractions were evaporated. The crude material was purified by flash chromatography on Biotage silica NH cartridge (from DCM to 2% MeOH), then it was purified by reverse flash chromatography on Biotage C18 cartridge (from H₂O+0.1% NH₄OH to 40% MeCN) to afford the title compound (46 mg, 0.08 mmol, 57% yield).

LC-MS (ESI): m/z (M+1): 560.2 (Method 2)

¹H NMR (500 MHz, Chloroform-d) δ ppm 9.31 (s, 1H), 8.21 (d, J=5.5 Hz, 1H), 8.14 (dd, J=6.7, 2.7 Hz, 1H), 8.07 (d, J=1.9 Hz, 1H), 7.74 (d, J=1.0 Hz, 1H), 7.40 (ddd, J=8.7, 4.2, 2.7 Hz, 1H), 7.13 (dd, J=10.5, 8.9 Hz, 1H), 6.92 (dd, J=5.5, 2.1 Hz, 1H), 6.52 (s, 1H), 4.07 (t, J=5.5 Hz, 2H), 3.66 (t, J=5.6 Hz, 2H), 3.35 (br s, 1H), 2.44-2.51 (m, 4H), 2.54 (br s, 8H), 2.31 (s, 3H), 1.92 (quin, J=6.7 Hz, 2H).

### Example 24: N-(4-{[6-(5-chloro-2-fluorophenyl)-3-[(2-hydroxyethyl)sulfanyl]pyridazin-4-yl]amino}pyridin-2-yl)-2-{6-methyl-2,6-diazaspiro[3.3]heptan-2-yl}acetamide

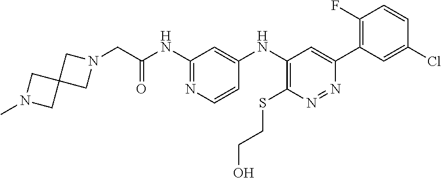 [SMILES: CN1CC2(C1)CN(CC(=O)Nc1cc(Nc3cc(-c4cc(Cl)ccc4F)nnc3SCCO)ccn1)C2]

Example 24 was prepared following the procedure used for the synthesis of Example 23, starting from Intermediate 73 (224 mg, 0.34 mmol) to afford title compound (67 mg, 0.12 mmol, 36% yield).

LC-MS (ESI): m/z (M+1): 544.2 (Method 2)

¹H NMR (600 MHz, Chloroform-d) δ ppm 9.48 (s, 1H), 8.25 (d, J=5.6 Hz, 1H), 8.14 (dd, J=6.6, 2.6 Hz, 1H), 8.09 (d, J=2.0 Hz, 1H), 7.74 (d, J=1.0 Hz, 1H), 7.36-7.43 (m, 1H), 7.13 (dd, J=10.5, 8.6 Hz, 1H), 6.95 (dd, J=5.6, 2.0 Hz, 1H), 6.53 (s, 1H), 4.07 (t, J=5.6 Hz, 2H), 3.66 (t, J=5.6 Hz, 2H), 3.51 (s, 4H), 3.33 (s, 4H), 3.26 (s, 2H), 2.30 (s, 3H).

### Example 25: N-(4-{[6-(5-chloro-2-fluorophenyl)-3-[(2-hydroxyethyl)sulfanyl]pyridazin-4-yl]amino}pyridin-2-yl)-2-{5-methyl-2,5-diazabicyclo[2.2.1]heptan-2-yl}acetamide

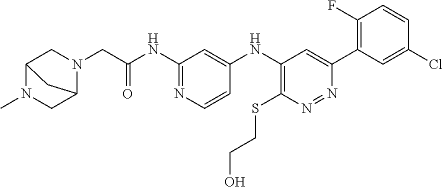 [SMILES: CN1CC2CC1CN2CC(=O)Nc1cc(Nc2cc(-c3cc(Cl)ccc3F)nnc2SCCO)ccn1]

Example 25 was prepared following the procedure used for the synthesis of Example 23, starting from Intermediate 76 (160 mg, 0.24 mmol) to afford title compound (88 mg, 0.16 mmol, 67% yield).

LC-MS (ESI): m/z (M+1): 544.2 (Method 2)

¹H NMR (400 MHz, Chloroform-d) δ ppm 9.74 (s, 1H), 8.25 (d, J=5.7 Hz, 1H), 8.15 (dd, J=6.7, 2.7 Hz, 1H), 8.12 (d, J=2.0 Hz, 1H), 7.75 (s, 1H), 7.40 (ddd, J=8.7, 4.2, 2.8 Hz, 1H), 7.14 (dd, J=10.4, 8.9 Hz, 1H), 6.95 (dd, J=5.7, 2.2 Hz, 1H), 6.54 (s, 1H), 4.08 (t, J=5.5 Hz, 2H), 3.67 (t, J=5.5 Hz, 2H), 3.31-3.45 (m, 3H), 3.30 (s, 1H), 3.21-3.45 (m, 1H), 2.84-2.95 (m, 2H), 2.68-2.84 (m, 2H), 2.42 (s, 3H), 1.74-1.94 (m, 2H).

### Example 26: N-(4-{[6-(5-chloro-2-fluorophenyl)-3-[(2-hydroxyethyl)sulfanyl]pyridazin-4-yl]amino}pyridin-2-yl)-2-methyl-2,8-diazaspiro[4.5]decane-8-carboxamide

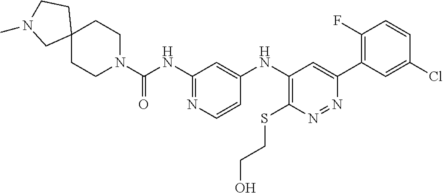 [SMILES: CN1CCC2(CCN(C(=O)Nc3cc(Nc4cc(-c5cc(Cl)ccc5F)nnc4SCCO)ccn3)CC2)C1]

Example 26 was prepared following the procedure used for the synthesis of Example 23, starting from Intermediate 81 (130 mg, 0.19 mmol) to afford title compound (53 mg, 0.09 mmol, 49% yield).

LC-MS (ESI): m/z (M+1): 572.2 (Method 2)

¹H NMR (400 MHz, Chloroform-d) δ ppm 8.09-8.20 (m, 2H), 7.90 (d, J=1.5 Hz, 1H), 7.72 (s, 1H), 7.40 (dt, J=8.6, 3.3 Hz, 1H), 7.20-7.32 (m, 1H), 7.13 (dd, J=10.3, 9.0 Hz, 1H), 6.86 (br dd, J=5.6, 1.9 Hz, 1H), 6.51 (s, 1H), 4.06 (t, J=5.5 Hz, 2H), 3.65 (t, J=5.5 Hz, 2H), 3.39-3.57 (m, 4H), 2.54-2.70 (m, 2H), 2.45 (s, 2H), 2.36 (s, 3H), 1.72 (br t, J=6.9 Hz, 2H), 1.45-1.68 (m, 4H).

### Example 27: N-(4-{[6-(5-chloro-2-fluorophenyl)-3-[(2-hydroxyethyl)sulfanyl]pyridazin-4-yl]amino}pyridin-2-yl)-2-(4-methyl-1,4-diazepan-1-yl)acetamide

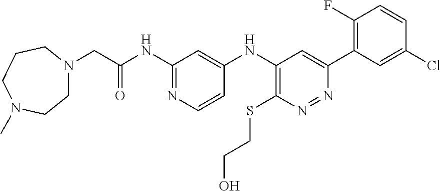 [SMILES: CN1CCCN(CC(=O)Nc2cc(Nc3cc(-c4cc(Cl)ccc4F)nnc3SCCO)ccn2)CC1]

Example 27 was prepared following the procedure used for the synthesis of Example 23, starting from Intermediate 83 (213 mg, 0.24 mmol) to afford title compound (43 mg, 0.08 mmol, 24% yield).

LC-MS (ESI): m/z (M+1): 546.2 (Method 2)

¹H NMR (400 MHz, Chloroform-d) δ ppm 9.78 (br s, 1H), 8.26 (d, J=5.7 Hz, 1H), 8.15 (dd, J=6.6, 2.7 Hz, 1H), 8.11 (d, J=2.0 Hz, 1H), 7.75 (s, 1H), 7.40 (dt, J=8.7, 3.4 Hz, 1H), 7.14 (dd, J=10.5, 9.0 Hz, 1H), 6.95 (dd, J=5.6, 2.0 Hz, 1H), 6.51 (s, 1H), 4.07 (br s, 2H), 3.67 (t, J=5.5 Hz, 2H), 3.32 (s, 2H), 3.19-3.29 (m, 1H), 2.84-2.97 (m, 4H), 2.64-2.79 (m, 4H), 2.41 (s, 3H), 1.90 (quin, J=5.8 Hz, 2H).

### Example 28: N-(4-{[6-(5-chloro-2-fluorophenyl)-3-[(3-hydroxypropyl)sulfanyl]pyridazin-4-yl]amino}pyridin-2-yl)-3-(4-methylpiperazin-1-yl)propanamide

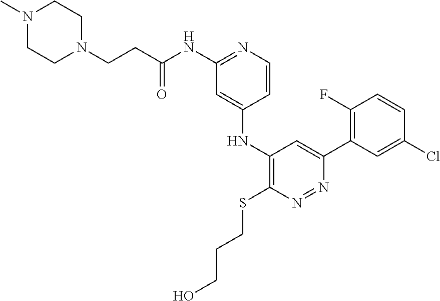 [SMILES: CN1CCN(CCC(=O)Nc2cc(Nc3cc(-c4cc(Cl)ccc4F)nnc3SCCCO)ccn2)CC1]

Example 28 was prepared following the procedure used for the synthesis of Example 23, starting from Intermediate 87 (150 mg, 0.19 mmol) to afford title compound (70 mg, 0.12 mmol, 66% yield).

LC-MS (ESI): m/z (M+1): 560.3 (Method 2)

¹H NMR (500 MHz, Chloroform-d) δ ppm 11.27 (br s, 1H), 8.24 (d, J=5.6 Hz, 1H), 8.16 (dd, J=6.7, 2.7 Hz, 1H), 8.05 (d, J=1.8 Hz, 1H), 7.72 (d, J=1.0 Hz, 1H), 7.39 (ddd, J=8.8, 4.3, 2.7 Hz, 1H), 7.13 (dd, J=10.6, 8.9 Hz, 1H), 6.92 (dd, J=5.6, 2.1 Hz, 1H), 6.40 (s, 1H), 3.82 (br s, 2H), 3.65 (t, J=6.6 Hz, 2H), 2.93-3.05 (m, 1H), 2.74-2.79 (m, 2H), 2.54-2.59 (m, 2H), 2.45-2.91 (m, 8H), 2.37 (s, 3H), 2.10 (quin, J=6.1 Hz, 2H).

### Example 29: N-(4-((6-(2-chloro-5-fluorophenyl)-3-(methylamino)pyridazin-4-yl)amino)pyridin-2-yl)-3-(4-methylpiperazin-1-yl)propenamide

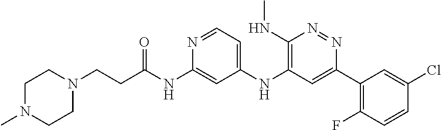 [SMILES: CNc1nnc(-c2cc(Cl)ccc2F)cc1Nc1ccnc(NC(=O)CCN2CCN(C)CC2)c1]

Example 29 was prepared following the procedure used for the synthesis of Example 1, starting from Intermediate 90 (100 mg, 0.396 mmol) and Intermediate 2 (129 mg, 0.396 mmol). Purification by reverse flash chromatography on Biotage C18 cartridge (from 100% H₂O/MeCN 95:5+0.1% HCOOH to 50% of MeCN/H₂O 95:5+0.1% HCCOH) afforded the title compound (37 mg, 0.074 mmol, 19% yield).

LC-MS (ESI): m/z (M+1): 499.1 (Method 1)

¹H NMR (600 MHz, DMSO-d6) δ ppm 10.47 (br s, 1H) 8.49 (s, 1H) 8.09 (d, J=5.77 Hz, 1H) 7.89-7.99 (m, 2H) 7.46-7.58 (m, 2H) 7.37 (dd, J=10.64, 8.85 Hz, 1H) 6.84 (dd, J=5.64, 2.05 Hz, 1H) 6.65-6.68 (m, 1H) 2.52-3.06 (m, 15H).

### Example 30: N-(4-{[6-(5-chloro-2-fluorophenyl)-3-(dimethylamino)pyridazin-4-yl]amino}pyridin-2-yl)-3-(4-methylpiperazin-1-yl)propanamide

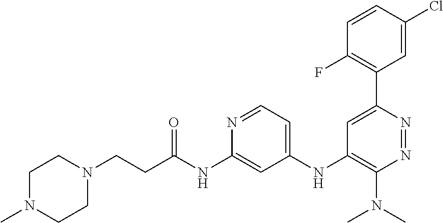 [SMILES: CN1CCN(CCC(=O)Nc2cc(Nc3cc(-c4cc(Cl)ccc4F)nnc3N(C)C)ccn2)CC1]

Example 30 was prepared following the procedure used for the synthesis of Example 1, starting from Intermediate 97 (100 mg, 0.375 mmol) and Intermediate 2 (123 mg, 0.375 mmol). Purification by reverse flash chromatography on Biotage C18 cartridge (from 100% H₂O/MeCN 95:5+0.1% HCOOH to 30% of MeCN/H₂O 95:5+0.1% HCCOH) afforded the title compound (21 mg, 0.041 mmol, 12% yield).

LC-MS (ESI): m/z (M+1): 513.1 (Method 1)

¹H NMR (600 MHz, DMSO-d6) δ ppm 10.57 (s, 1H) 8.91 (s, 1H) 8.09 (d, J=5.64 Hz, 1H) 8.01 (s, 1H) 7.97 (dd, J=6.60, 2.76 Hz, 1H) 7.66 (s, 1H) 7.53-7.59 (m, 1H) 7.41 (dd, J=10.51, 8.85 Hz, 1H) 6.87 (dd, J=5.64, 2.18 Hz, 1H) 2.93 (s, 6H) 2.60 (br t, J=6.92 Hz, 3H) 2.17-2.49 (m, 8H) 2.15 (s, 3H).

### Example 31: N-(4-{[6-(5-chloro-2-fluorophenyl)-3-(2-methoxyethoxy)pyridazin-4-yl]amino}pyridin-2-yl)-3-(4-methylpiperazin-1-yl)propanamide

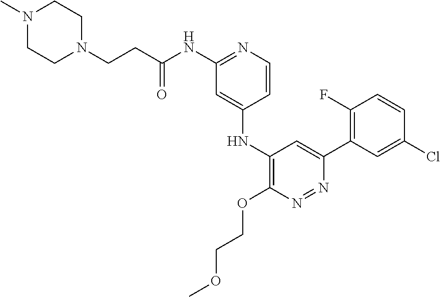 [SMILES: COCCOc1nnc(-c2cc(Cl)ccc2F)cc1Nc1ccnc(NC(=O)CCN2CCN(C)CC2)c1]

Example 31 was prepared following the procedure used for the synthesis of Example 1, starting from 6-(5-chloro-2-fluorophenyl)-3-(2-methoxyethoxy)pyridazin-4-amine (Intermediate 107, 36 mg, 0.12 mmol) and N-(4-bromopyridin-2-yl)-3-(4-methylpiperazin-1-yl)propanamide (Intermediate 2, 44 mg, 0.13 mmol) to afford title compound (41 mg, 0.07 mmol, 62% yield).

LC-MS (ESI): m/z (M+1): 544.1 (Method 2)

¹H NMR (400 MHz, Chloroform-d) δ ppm 11.18 (s, 1H), 8.23 (d, J=5.7 Hz, 1H), 8.11 (dd, J=6.8, 2.8 Hz, 1H), 8.08 (d, J=2.2 Hz, 1H), 7.77 (d, J=1.3 Hz, 1H), 7.37 (ddd, J=8.8, 4.2, 2.9 Hz, 1H), 7.13 (dd, J=10.5, 8.8 Hz, 1H), 7.04 (s, 1H), 6.96 (dd, J=5.7, 2.2 Hz, 1H), 4.78-4.89 (m, 2H), 3.86-3.95 (m, 2H), 3.48 (s, 3H), 2.73-2.81 (m, 2H), 2.52-2.60 (m, 2H), 2.42-3.01 (m, 8H), 2.38 (s, 3H).

### Example 32: N-[6-(5-chloro-2-fluorophenyl)-3-(2-methoxyethoxy)pyridazin-4-yl]-7-[2-(4-methylpiperazin-1-yl)ethoxy]quinolin-4-amine

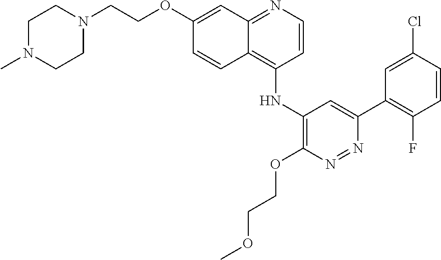 [SMILES: COCCOc1nnc(-c2cc(Cl)ccc2F)cc1Nc1ccnc2cc(OCCN3CCN(C)CC3)ccc12]

Example 32 was prepared following the procedure used for the synthesis of Example 2, starting from 4-chloro-7-[2-(4-methylpiperazin-1-yl)ethoxy]quinoline (Intermediate 5, 74 mg, 0.24 mmol) and 6-(5-chloro-2-fluorophenyl)-3-(2-methoxyethoxy)pyridazin-4-amine (Intermediate 107, 60 mg, 0.20 mmol) to afford title compound (37 mg, 0.06 mmol, 32% yield).

LC-MS (ESI): m/z (M+1): 567.1 (Method 2)

¹H NMR (500 MHz, Chloroform-d) δ ppm 8.80 (d, J=5.0 Hz, 1H), 8.08 (dd, J=6.7, 2.7 Hz, 1H), 7.87 (d, J=9.2 Hz, 1H), 7.64 (d, J=1.5 Hz, 1H), 7.46 (d, J=2.4 Hz, 1H), 7.31-7.41 (m, 3H), 7.24-7.31 (m, 1H), 7.10 (dd, J=10.4, 8.9 Hz, 1H), 4.86-4.99 (m, 2H), 4.30 (t, J=5.7 Hz, 2H), 3.88-4.00 (m, 2H), 3.50 (s, 3H), 2.93 (t, J=5.6 Hz, 2H), 2.38-2.84 (m, 8H), 2.31 (s, 3H).

### Example 33: N-(4-{[6-(5-chloro-2-fluorophenyl)-3-(2-methoxyethoxy)pyridazin-4-yl]amino}pyridin-2-yl)-3-[4-(2,2,2-trifluoroethyl)piperazin-1-yl]propanamide

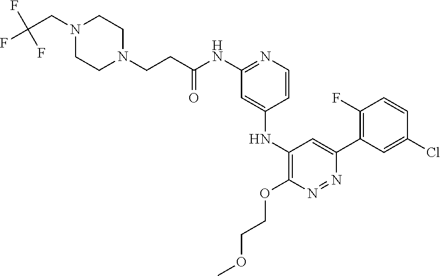 [SMILES: COCCOc1nnc(-c2cc(Cl)ccc2F)cc1Nc1ccnc(NC(=O)CCN2CCN(CC(F)(F)F)CC2)c1]

Example 33 was prepared following the procedure used for the synthesis of Example 1, starting from 6-(5-chloro-2-fluorophenyl)-3-(2-methoxyethoxy)pyridazin-4-amine (Intermediate 107, 60 mg, 0.20 mmol) and N-(4-bromopyridin-2-yl)-3-[4-(2,2,2-trifluoroethyl)piperazin-1-yl]propanamide (Intermediate 38, 89 mg, 0.22 mmol) to afford title compound (75 mg, 0.12 mmol, 71% yield).

LC-MS (ESI): m/z (M+1): 612.5 (Method 2)

¹H NMR (400 MHz, Chloroform-d) δ ppm11.11 (br. s, 1H), 8.22 (d, J=5.7 Hz, 1H), 8.10 (dd, J=6.6, 2.6 Hz, 1H), 8.08 (br. d, J=1.5 Hz, 1H), 7.77 (s, 1H), 7.37 (ddd, J=8.7, 3.9, 3.2 Hz, 1H), 7.06-7.20 (m, 2H), 6.96 (dd, J=5.6, 1.9 Hz, 1H), 4.76-4.94 (m, 2H), 3.84-3.97 (m, 2H), 3.48 (s, 3H), 3.04 (q, J=9.4 Hz, 2H), 2.86 (br. t, J=4.2 Hz, 4H), 2.73-2.80 (m, 2H), 2.68 (br. s, 4H), 2.48-2.60 (m, 2H).

### Example 34: N-(4-{[6-(5-chloro-2-fluorophenyl)-3-(2-methoxyethoxy)pyridazin-4-yl]amino}pyridin-2-yl)-3-(morpholin-4-yl)propanamide

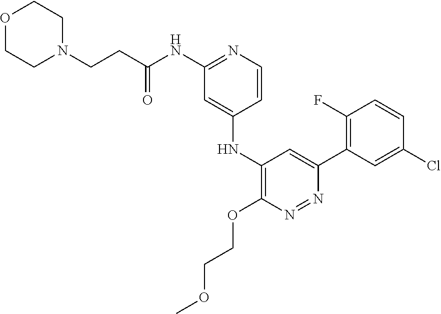 [SMILES: COCCOc1nnc(-c2cc(Cl)ccc2F)cc1Nc1ccnc(NC(=O)CCN2CCOCC2)c1]

Example 34 was prepared following the procedure used for the synthesis of Example 1, starting from 6-(5-chloro-2-fluorophenyl)-3-(2-methoxyethoxy)pyridazin-4-amine (Intermediate 107, 60 mg, 0.20 mmol) and N-(4-bromopyridin-2-yl)-3-(morpholin-4-yl)propanamide (Intermediate 108, 70 mg, 0.22 mmol) to afford title compound (52 mg, 0.10 mmol, 49% yield).

LC-MS (ESI): m/z (M+1): 531.1 (Method 2)

¹H NMR (400 MHz, Chloroform-d) δ ppm 11.11 (s, 1H), 8.22 (d, J=5.7 Hz, 1H), 8.11 (dd, J=6.7, 2.7 Hz, 1H), 8.08 (d, J=2.0 Hz, 1H), 7.78 (d, J=1.3 Hz, 1H), 7.37 (ddd, J=8.8, 4.2, 2.9 Hz, 1H), 7.13 (dd, J=10.4, 8.9 Hz, 1H), 7.05 (s, 1H), 6.96 (dd, J=5.6, 2.1 Hz, 1H), 4.75-4.91 (m, 2H), 3.82-3.97 (m, 6H), 3.48 (s, 3H), 2.73-2.82 (m, 2H), 2.64 (br. s, 4H), 2.54-2.60 (m, 2H).

### Example 35: N-[6-(5-chloro-2-fluorophenyl)-3-[2-(4-methylpiperazin-1-yl)ethoxy]pyridazin-4-yl]-7-methoxyquinolin-4-amine

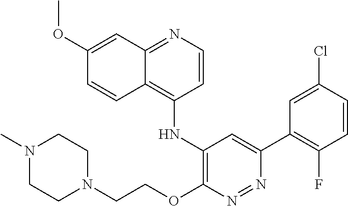 [SMILES: COc1ccc2c(Nc3cc(-c4cc(Cl)ccc4F)nnc3OCCN3CCN(C)CC3)ccnc2c1]

Example 35 was prepared following the procedure used for the synthesis of Example 2, starting from 6-(5-chloro-2-fluorophenyl)-3-[2-(4-methylpiperazin-1-yl)ethoxy]pyridazin-4-amine (Intermediate 111, 60 mg, 0.16 mmol) and 4-chloro-7-methoxyquinoline (35 mg, 0.18 mmol) to afford title compound (45 mg, 0.09 mmol, 52% yield).

LC-MS (ESI): m/z (M+1): 523.3 (Method 2)

¹H NMR (500 MHz, Chloroform-d) δ ppm 8.79 (d, J=4.9 Hz, 1H), 8.02-8.11 (m, 2H), 7.93 (d, J=9.3 Hz, 1H), 7.62 (d, J=1.8 Hz, 1H), 7.47 (d, J=2.5 Hz, 1H), 7.34 (s, 1H), 7.33 (d, J=5.1 Hz, 1H), 7.25 (dd, J=9.3, 2.5 Hz, 1H), 7.09 (dd, J=10.5, 8.9 Hz, 1H), 4.85 (s, 2H), 3.99 (s, 3H), 2.98 (s, 2H), 2.37-2.90 (m, 8H), 2.31 (s, 3H).

### Example 36: N-(4-{[6-(5-chloro-2-fluorophenyl)-3-[2-(4-methylpiperazin-1-yl)ethoxy]pyridazin-4-yl]amino}pyridin-2-yl)cyclopropanecarboxamide

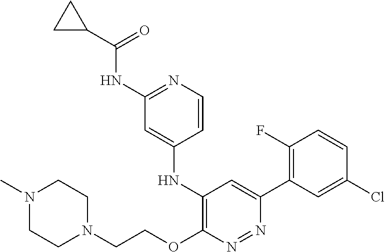 [SMILES: CN1CCN(CCOc2nnc(-c3cc(Cl)ccc3F)cc2Nc2ccnc(NC(=O)C3CC3)c2)CC1]

Example 36 was prepared following the procedure used for the synthesis of Example 1, starting from 6-(5-chloro-2-fluorophenyl)-3-[2-(4-methylpiperazin-1-yl)ethoxy]pyridazin-4-amine (Intermediate 111, 60 mg, 0.16 mmol) and N-(4-bromopyridin-2-yl)cyclopropanecarboxamide (Intermediate 112, 43 mg, 0.18 mmol) to afford title compound (25 mg, 0.05 mmol, 29% yield).

LC-MS (ESI): m/z (M+1): 526.3 (Method 2)

¹H NMR (500 MHz, Chloroform-d) δ ppm 8.34 (br. s, 1H), 8.23 (s, 1H), 8.18 (d, J=5.6 Hz, 1H), 8.04-8.12 (m, 2H), 7.76 (d, J=1.4 Hz, 1H), 7.32-7.40 (m, 1H), 7.12 (dd, J=10.5, 8.9 Hz, 1H), 7.01 (dd, J=5.7, 2.1 Hz, 1H), 4.77 (t, J=5.5 Hz, 2H), 2.92 (t, J=5.5 Hz, 2H), 2.32 (s, 3H), 2.14-3.00 (m, 8H), 1.50-1.63 (m, 1H), 1.08-1.16 (m, 2H), 0.89-0.95 (m, 2H).

### Example 37: 6-(5-chloro-2-fluorophenyl)-3-[2-(4-methylpiperazin-1-yl)ethoxy]-N-{1H-pyrrolo[2,3-b]pyridin-4-yl}pyridazin-4-amine

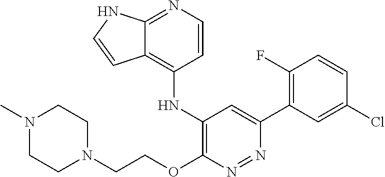 [SMILES: CN1CCN(CCOc2nnc(-c3cc(Cl)ccc3F)cc2Nc2ccnc3[nH]ccc23)CC1]

Example 37 was prepared following the procedure used for the synthesis of Example 10, starting from 6-(5-chloro-2-fluorophenyl)-3-[2-(4-methylpiperazin-1-yl)ethoxy]-N-(1-{[2-(trimethylsilyl)ethoxy]methyl}-1H-pyrrolo[2,3-b]pyridin-4-yl)pyridazin-4-amine (Intermediate 114, 79 mg, 0.13 mmol) to afford title compound (52 mg, 0.11 mmol, 84% yield).

LC-MS (ESI): m/z (M+1): 482.3 (Method 2)

¹H NMR (500 MHz, Chloroform-d) δ ppm 9.61 (br. s, 1H), 8.29 (d, J=5.4 Hz, 1H), 8.08 (dd, J=6.6, 2.7 Hz, 1H), 7.83 (s, 1H), 7.68 (d, J=1.5 Hz, 1H), 7.35 (ddd, J=8.8, 4.1, 2.7 Hz, 1H), 7.32 (dd, J=3.3, 2.3 Hz, 1H), 7.06-7.12 (m, 2H), 6.52 (dd, J=3.4, 1.5 Hz, 1H), 4.85 (t, J=5.5 Hz, 2H), 2.98 (t, J=5.6 Hz, 2H), 2.35-2.94 (m, 8H), 2.32 (s, 3H).

### Example 38: N-(4-{[6-(5-chloro-2-fluorophenyl)-3-[2-(dimethylamino)ethoxy]pyridazin-4-yl]amino}pyridin-2-yl)cyclopropanecarboxamide

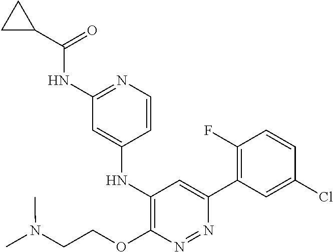 [SMILES: CN(C)CCOc1nnc(-c2cc(Cl)ccc2F)cc1Nc1ccnc(NC(=O)C2CC2)c1]

Example 38 was prepared following the procedure used for the synthesis of Example 1, starting from 6-(5-chloro-2-fluorophenyl)-3-[2-(dimethylamino)ethoxy]pyridazin-4-amine (Intermediate 30, 60 mg, 0.19 mmol) and N-(4-bromopyridin-2-yl)cyclopropanecarboxamide (Intermediate 112, 51 mg, 0.21 mmol) to afford title compound (54 mg, 0.11 mmol, 69% yield).

LC-MS (ESI): m/z (M+1): 471.3 (Method 2)

¹H NMR (600 MHz, Chloroform-d) δ ppm 8.74 (s, 1H), 8.22 (s, 1H), 8.16 (d, J=5.9 Hz, 1H), 8.13 (d, J=2.0 Hz, 1H), 8.08 (dd, J=6.6, 2.6 Hz, 1H), 7.76 (d, J=1.3 Hz, 1H), 7.36 (ddd, J=8.6, 4.2, 2.6 Hz, 1H), 7.08-7.16 (m, 1H), 7.02 (dd, J=5.6, 2.3 Hz, 1H), 4.65-4.79 (m, 2H), 2.79-2.93 (m, 2H), 2.36 (s, 6H), 1.49-1.61 (m, 1H), 1.07-1.15 (m, 2H), 0.87-0.95 (m, 2H).

### Example 39: N-[6-(5-chloro-2-fluorophenyl)-3-[2-(dimethylamino)ethoxy]pyridazin-4-yl]-7-methoxyquinolin-4-amine

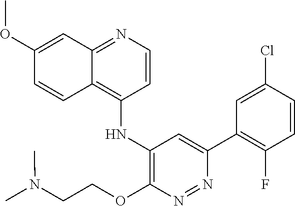 [SMILES: COc1ccc2c(Nc3cc(-c4cc(Cl)ccc4F)nnc3OCCN(C)C)ccnc2c1]

Example 39 was prepared following the procedure used for the synthesis of Example 2, starting from 6-(5-chloro-2-fluorophenyl)-3-[2-(dimethylamino)ethoxy]pyridazin-4-amine (Intermediate 30, 60 mg, 0.19 mmol) and 4-chloro-7-methoxyquinoline (41 mg, 0.21) to afford title compound (46 mg, 0.10 mmol, 51% yield).

LC-MS (ESI): m/z (M+1): 468.3 (Method 2)

¹H NMR (600 MHz, Chloroform-d) δ ppm 8.78 (d, J=4.9 Hz, 1H), 8.30 (br. s, 1H), 8.06 (dd, J=6.6, 2.6 Hz, 1H), 7.97 (d, J=9.2 Hz, 1H), 7.57 (d, J=1.6 Hz, 1H), 7.46 (d, J=2.6 Hz, 1H), 7.33-7.37 (m, 1H), 7.32 (d, J=4.9 Hz, 1H), 7.23 (dd, J=9.2, 2.6 Hz, 1H), 7.08 (dd, J=10.4, 8.7 Hz, 1H), 4.75-4.90 (m, 2H), 3.98 (s, 3H), 2.85-2.95 (m, 2H), 2.39 (s, 6H).

### Example 40: N-(4-{[6-(5-chloro-2-fluorophenyl)-3-[2-(dimethylamino)ethoxy]pyridazin-4-yl]amino}pyridin-2-yl)-3-(morpholin-4-yl)propanamide

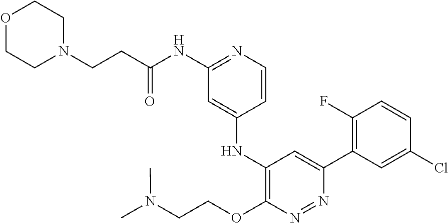 [SMILES: CN(C)CCOc1nnc(-c2cc(Cl)ccc2F)cc1Nc1ccnc(NC(=O)CCN2CCOCC2)c1]

Example 40 was prepared following the procedure used for the synthesis of Example 1, starting from 6-(5-chloro-2-fluorophenyl)-3-[2-(dimethylamino)ethoxy]pyridazin-4-amine (Intermediate 30, 40 mg, 0.13 mmol) and N-(4-bromopyridin-2-yl)-3-(morpholin-4-yl)propanamide (Intermediate 108, 44 mg, 0.14 mmol) to afford title compound (22 mg, 0.04 mmol, 31% yield).

LC-MS (ESI): m/z (M+1): 544.4 (Method 2)

¹H NMR (400 MHz, Chloroform-d) δ ppm 10.95 (br. s, 1H), 8.50 (s, 1H), 8.19 (d, J=5.7 Hz, 1H), 8.13 (d, J=1.8 Hz, 1H), 8.09 (dd, J=6.8, 2.9 Hz, 1H), 7.77 (d, J=1.3 Hz, 1H), 7.36 (ddd, J=8.8, 4.2, 2.9 Hz, 1H), 7.12 (dd, J=10.5, 8.8 Hz, 1H), 6.99 (dd, J=5.7, 2.2 Hz, 1H), 4.67-4.79 (m, 2H), 3.86 (t, J=4.4 Hz, 4H), 2.82-2.91 (m, 2H), 2.73-2.81 (m, 2H), 2.59-2.67 (m, 4H), 2.53-2.59 (m, 2H), 2.37 (s, 6H).

### Example 41: 6-(5-chloro-2-fluorophenyl)-3-[2-(dimethylamino)ethoxy]-N-{1H-pyrrolo[2,3-b]pyridin-4-yl}pyridazin-4-amine

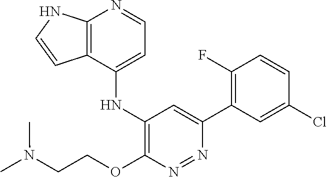 [SMILES: CN(C)CCOc1nnc(-c2cc(Cl)ccc2F)cc1Nc1ccnc2[nH]ccc12]

Example 41 was prepared following the procedure used for the synthesis of Example 10, starting from 6-(5-chloro-2-fluorophenyl)-3-[2-(dimethylamino)ethoxy]-N-(1-{[2-(trimethylsilyl)ethoxy]methyl}-1H-pyrrolo[2,3-b]pyridin-4-yl)pyridazin-4-amine (Intermediate 115, 84 mg, 0.15 mmol) to afford title compound (38 mg, 0.09 mmol, 59% yield).

LC-MS (ESI): m/z (M+1): 427.2 (Method 2)

¹H NMR (500 MHz, Chloroform-d) δ ppm 9.44 (br. s, 1H), 8.27 (d, J=5.4 Hz, 1H), 8.13 (br. s, 1H), 8.07 (dd, J=6.7, 2.7 Hz, 1H), 7.64 (d, J=1.5 Hz, 1H), 7.34 (ddd, J=8.7, 4.1, 2.8 Hz, 1H), 7.29 (dd, J=3.2, 2.3 Hz, 1H), 7.05-7.11 (m, 2H), 6.51 (dd, J=3.3, 1.5 Hz, 1H), 4.81 (t, J=5.4 Hz, 2H), 2.91 (t, J=5.4 Hz, 2H), 2.41 (s, 6H).

### Example 42: 6-(5-chloro-2-fluorophenyl)-3-[2-(dimethylamino)ethoxy]-N-{1H-pyrazolo[3,4-b]pyridin-4-yl}pyridazin-4-amine

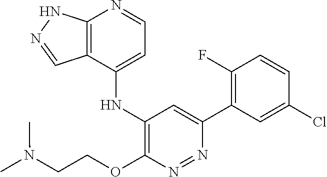 [SMILES: CN(C)CCOc1nnc(-c2cc(Cl)ccc2F)cc1Nc1ccnc2[nH]ncc12]

Example 42 was prepared following the procedure used for the synthesis of Example 10, starting from 6-(5-chloro-2-fluorophenyl)-3-[2-(dimethylamino)ethoxy]-N-(1-{[2-(trimethylsilyl)ethoxy]methyl}-1H-pyrazolo[3,4-b]pyridin-4-yl)pyridazin-4-amine (Intermediate 117, 113 mg, 0.20 mmol) to afford title compound (73 mg, 0.17 mmol, 84% yield).

LC-MS (ESI): m/z (M+1): 428.4 (Method 2)

¹H NMR (500 MHz, DMSO-d₆) δ ppm 13.56 (br. s, 1H), 9.24 (br. s, 1H), 8.34 (d, J=5.2 Hz, 1H), 8.11 (s, 1H), 7.93 (dd, J=6.6, 2.7 Hz, 1H), 7.64 (d, J=1.4 Hz, 1H), 7.51-7.62 (m, 1H), 7.40 (dd, J=10.6, 8.9 Hz, 1H), 6.99 (d, J=5.2 Hz, 1H), 4.69 (t, J=6.1 Hz, 2H), 2.77 (t, J=6.1 Hz, 2H), 2.23 (s, 6H).

### Example 43: 2-({4-[(2-aminopyridin-4-yl)amino]-6-(5-chloro-2-fluorophenyl)pyridazin-3-yl}oxy)ethan-1-ol

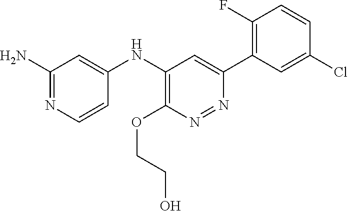 [SMILES: Nc1cc(Nc2cc(-c3cc(Cl)ccc3F)nnc2OCCO)ccn1]

TFA (1 mL, 13.1 mmol) was added to a stirred mixture of tert-butyl N-(4-{[6-(5-chloro-2-fluorophenyl)-3-(2-hydroxyethoxy)pyridazin-4-yl]amino}pyridin-2-yl)carbamate (Intermediate 118, 90 mg, 0.19 mmol) in DCM at 0° C. under N₂. After 30 minutes the reaction was warmed at RT and stirred for 3 hrs. Volatiles were removed by reduced pressure, the residue was dissolved with DCM and washed with saturated NaHCO₃ solution, Organic layer was separated, dried over Na₂SO₄ and evaporated. The residue was purified by flash chromatography on Biotage silica cartridge (from DCM to 5% MeOH/0.5% H₂O) to afford title compound (40 mg, 0.11 mmol, 56% yield).

LC-MS (ESI): m/z (M+1): 376.1 (Method 2)

¹H NMR (400 MHz, DMSO-d₆) δ ppm 8.39-8.60 (m, 1H), 7.91 (dd, J=6.6, 2.6 Hz, 1H), 7.84 (d, J=5.7 Hz, 1H), 7.53-7.62 (m, 2H), 7.42 (dd, J=10.4, 8.9 Hz, 1H), 6.50 (dd, J=5.6, 1.9 Hz, 1H), 6.38 (d, J=1.5 Hz, 1H), 5.90 (s, 2H), 5.01 (br. s, 1H), 4.53 (t, J=4.7 Hz, 2H), 3.85 (br. d, J=3.9 Hz, 2H).

### Example 44: N-(4-{[6-(5-chloro-2-fluorophenyl)-3-[(1-methylazetidin-3-yl)methoxy]pyridazin-4-yl]amino}pyridin-2-yl)cyclopropanecarboxamide

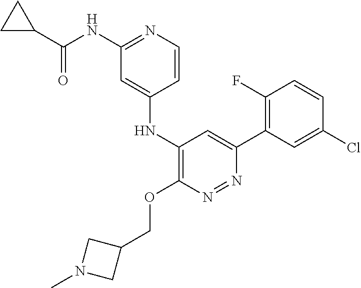 [SMILES: CN1CC(COc2nnc(-c3cc(Cl)ccc3F)cc2Nc2ccnc(NC(=O)C3CC3)c2)C1]

Example 44 was prepared following the procedure used for the synthesis of Example 1, starting from 6-(5-chloro-2-fluorophenyl)-3-[(1-methylazetidin-3-yl)methoxy]pyridazin-4-amine (Intermediate 121, 85 mg, 0.26 mmol) and N-(4-bromopyridin-2-yl)cyclopropanecarboxamide (Intermediate 112, 73 mg, 0.29 mmol) to afford title compound (25 mg, 0.05 mmol, 20% yield).

LC-MS (ESI): m/z (M+1): 483.4 (Method 2)

¹H NMR (500 MHz, Chloroform-d) δ ppm 8.20 (d, J=5.9 Hz, 1H), 8.11 (br. s, 1H), 8.09 (dd, J=6.7, 2.8 Hz, 1H), 8.07 (d, J=2.0 Hz, 1H), 7.76 (d, J=1.5 Hz, 1H), 7.52 (br. s, 1H), 7.34-7.41 (m, 1H), 7.12 (dd, J=10.5, 8.8 Hz, 1H), 7.00 (dd, J=5.7, 2.1 Hz, 1H), 4.81 (d, J=6.1 Hz, 2H), 3.46 (t, J=7.8 Hz, 2H), 3.27 (br. s, 2H), 2.94-3.08 (m, 1H), 2.40 (s, 3H), 1.52-1.58 (m, 1H), 1.10-1.15 (m, 2H), 0.89-0.98 (m, 2H).

### Example 45: N-[2-({4-[(2-aminopyridin-4-yl)amino]-6-(5-chloro-2-fluorophenyl)pyridazin-3-yl}oxy)ethyl]methanesulfonamide

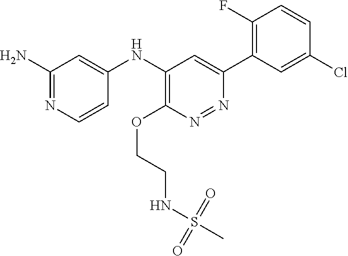 [SMILES: CS(=O)(=O)NCCOc1nnc(-c2cc(Cl)ccc2F)cc1Nc1ccnc(N)c1]

Example 45 was prepared following the procedure used for the synthesis of Example 19, starting from tert-butyl N-[2-({4-[(2-{[(tert-butoxy)carbonyl]amino}pyridin-4-yl)amino]-6-(5-chloro-2-fluorophenyl)pyridazin-3-yl}oxy)ethyl]-N-methanesulfonylcarbamate (Intermediate 122, 0.14 mmol) to afford title compound (26 mg, 0.06 mmol, 43% yield).

LC-MS (ESI): m/z (M+1): 453.2 (Method 1)

¹H NMR (400 MHz, DMSO-d₆) δ ppm 8.42 (br. s, 1H), 7.90 (dd, J=6.6, 2.9 Hz, 1H), 7.85 (d, J=5.7 Hz, 1H), 7.56-7.63 (m, 1H), 7.55 (d, J=1.3 Hz, 1H), 7.38-7.50 (m, 2H), 6.49 (dd, J=5.6, 1.9 Hz, 1H), 6.37 (d, J=1.5 Hz, 1H), 5.92 (s, 2H), 4.58 (t, J=5.2 Hz, 2H), 3.50 (br. t, J=4.3 Hz, 2H), 3.00 (s, 3H).

### Example 46: N-[6-(5-chloro-2-fluorophenyl)-3-methoxypyridazin-4-yl]-7-[2-(4-methylpiperazin-1-yl)ethoxy]quinolin-4-amine

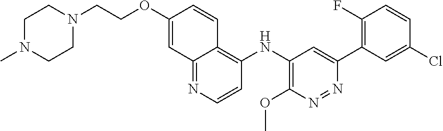 [SMILES: COc1nnc(-c2cc(Cl)ccc2F)cc1Nc1ccnc2cc(OCCN3CCN(C)CC3)ccc12]

Example 46 was prepared following the procedure used for the synthesis of Example 2, starting from 4-chloro-7-[2-(4-methylpiperazin-1-yl)ethoxy]quinoline (Intermediate 5, 72 mg, 0.23 mmol) and 6-(5-chloro-2-fluorophenyl)-3-methoxypyridazin-4-amine (Intermediate 37, 55 mg, 0.22 mmol) to afford title compound (20 mg, 0.04 mmol, 20% yield).

LC-MS (ESI): m/z (M+1): 523.4 (Method 2)

¹H NMR (400 MHz, Chloroform-d) δ ppm 8.80 (d, J=5.0 Hz, 1H), 8.10 (dd, J=6.7, 2.7 Hz, 1H), 7.87 (d, J=9.2 Hz, 1H), 7.68 (d, J=1.6 Hz, 1H), 7.47 (d, J=2.5 Hz, 1H), 7.37 (ddd, J=6.6, 4.3, 2.1 Hz, 1H), 7.34 (d, J=5.3 Hz, 1H), 7.26-7.31 (m, 1H), 7.22 (s, 1H), 7.10 (dd, J=10.6, 8.8 Hz, 1H), 4.37 (s, 3H), 4.30 (t, J=5.7 Hz, 2H), 2.93 (t, J=5.7 Hz, 2H), 2.38-2.82 (m, 8H), 2.31 (s, 3H).

### Example 47: N-(4-{[6-(5-chloro-2-fluorophenyl)-3-methoxypyridazin-4-yl]amino}pyridin-2-yl)-3-(4-methylpiperazin-1-yl)propanamide

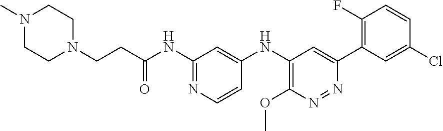 [SMILES: COc1nnc(-c2cc(Cl)ccc2F)cc1Nc1ccnc(NC(=O)CCN2CCN(C)CC2)c1]

Example 47 was prepared following the procedure used for the synthesis of Example 1, starting from 6-(5-chloro-2-fluorophenyl)-3-methoxypyridazin-4-amine (Intermediate 37, 50 mg, 0.16 mmol) and N-(4-bromopyridin-2-yl)-3-(4-methylpiperazin-1-yl)propanamide (Intermediate 2, 57 mg, 0.17 mmol) to afford title compound (20 mg, 0.04 mmol, 25% yield).

LC-MS (ESI): m/z (M+1): 500.4 (Method 2)

¹H NMR (600 MHz, Chloroform-d) δ ppm 11.20 (s, 1H), 8.24 (d, J=5.6 Hz, 1H), 8.11 (dd, J=6.7, 2.7 Hz, 1H), 8.07 (d, J=2.0 Hz, 1H), 7.76 (d, J=1.3 Hz, 1H), 7.37 (ddd, J=8.8, 4.2, 2.7 Hz, 1H), 7.13 (dd, J=10.5, 8.7 Hz, 1H), 6.95 (dd, J=5.7, 2.2 Hz, 1H), 6.94 (s, 1H), 4.30 (s, 3H), 2.74-2.80 (m, 2H), 2.55-2.58 (m, 2H), 2.44-2.84 (m, 8H), 2.37 (s, 3H).

### Example 48: N4-[6-(5-chloro-2-fluorophenyl)-3-methoxypyridazin-4-yl]pyridine-2,4-diamine

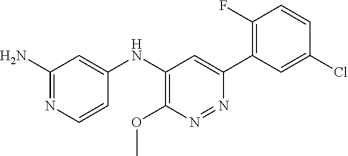 [SMILES: COc1nnc(-c2cc(Cl)ccc2F)cc1Nc1ccnc(N)c1]

In a suitable vial, a mixture of tert-butyl N-(4-bromopyridin-2-yl)carbamate (59 mg, 0.21 mmol), Cs₂CO₃ (128 mg, 0.39 mmol), XantPhos (14 mg, 0.02 mmol), 6-(5-chloro-2-fluorophenyl)-3-methoxypyridazin-4-amine (Intermediate 37, 55 mg, 0.20 mmol) and Pd(OAc)₂ (2.2 mg, 0.01 mmol) was suspended in dry 1,4-Dioxane (2 mL). The vial was sealed, evacuated, backfilled with N₂ (3 times), and heated at 100° C. overnight. The mixture was diluted with EtOAc, filtered through a Celite® pad, washing with EtOAc. The residue was suspended with DCM (3 mL) and TFA (0.3 mL, 3.9 mmol) was added. The dark brown mixture was stirred at RT for 3 hrs. Volatiles were removed under vacuum and the residue was charged in SCX washing with MeOH and eluting with 2 N NH₃ in MeOH. Evaporation of basic fraction afforded a residue that was purified by reverse flash chromatography on Biotage C18 cartridge (from H₂O 0.1% HCOOH to 20% MeCN+0.1% HCOOH). Opportune fractions were collected and evaporated, then dissolved with MeOH and passed through a PL-HCO₃ cartridge, evaporation of solvent afforded title compound (7.8 mg, 0.02 mmol, 12% yield).

LC-MS (ESI): m/z (M+1): 346.1 (Method 1)

¹H NMR (400 MHz, DMSO-d₆) δ ppm 8.71 (s, 1H), 7.91 (dd, J=6.6, 2.6 Hz, 1H), 7.80 (d, J=5.7 Hz, 1H), 7.51-7.63 (m, 2H), 7.42 (dd, J=10.3, 9.0 Hz, 1H), 6.50 (dd, J=5.7, 1.8 Hz, 1H), 6.38 (d, J=1.8 Hz, 1H), 5.84 (s, 2H), 4.16 (s, 3H).

### Example 49: N4-[6-(5-chloro-2-fluorophenyl)-3-(2,2,2-trifluoroethoxy)pyridazin-4-yl]pyridine-2,4-diamine

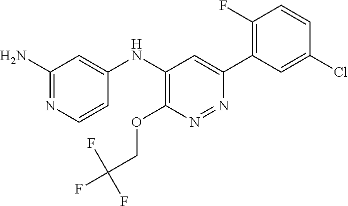 [SMILES: Nc1cc(Nc2cc(-c3cc(Cl)ccc3F)nnc2OCC(F)(F)F)ccn1]

Example 49 was prepared following the procedure used for the synthesis of Example 43, starting from tert-butyl N-(4-{[6-(5-chloro-2-fluorophenyl)-3-(2,2,2-trifluoroethoxy)pyridazin-4-yl]amino}pyridin-2-yl)carbamate (Intermediate 126, 50 mg, 0.10 mmol) to afford title compound (30 mg, 0.07 mmol, 75% yield).

LC-MS (ESI): m/z (M+1): 414.3 (Method 2)

¹H NMR (400 MHz, Chloroform-d) δ ppm 8.01-8.12 (m, 2H), 7.75 (d, J=1.5 Hz, 1H), 7.40 (ddd, J=8.8, 4.3, 2.7 Hz, 1H), 7.14 (dd, J=10.6, 8.9 Hz, 1H), 6.52-6.67 (m, 2H), 6.36 (d, J=1.8 Hz, 1H), 5.08 (q, J=8.3 Hz, 2H), 4.53 (br. s, 2H).

### Example 50: N-(4-{[6-(5-chloro-2-fluorophenyl)-3-(2,2,2-trifluoroethoxy)pyridazin-4-yl]amino}pyridin-2-yl)-3-(4-methylpiperazin-1-yl)propanamide

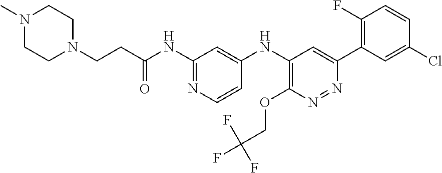 [SMILES: CN1CCN(CCC(=O)Nc2cc(Nc3cc(-c4cc(Cl)ccc4F)nnc3OCC(F)(F)F)ccn2)CC1]

Example 50 was prepared following the procedure used for the synthesis of Example 1, starting from 6-(5-chloro-2-fluorophenyl)-3-(2,2,2-trifluoroethoxy)pyridazin-4-amine (Intermediate 125, 55 mg, 0.17 mmol) and N-(4-bromopyridin-2-yl)-3-(4-methylpiperazin-1-yl)propanamide (Intermediate 2, 62 mg, 0.19 mmol) to afford title compound (29 mg, 0.05 mmol, 30% yield).

LC-MS (ESI): m/z (M+1): 568.4 (Method 2)

¹H NMR (400 MHz, Chloroform-d) δ ppm11.25 (br. s, 1H), 8.26 (d, J=5.6 Hz, 1H), 8.12 (d, J=2.0 Hz, 1H), 8.08 (dd, J=6.6, 2.7 Hz, 1H), 7.82 (d, J=1.3 Hz, 1H), 7.39 (ddd, J=8.8, 4.2, 2.8 Hz, 1H), 7.14 (dd, J=10.5, 8.8 Hz, 1H), 6.95 (dd, J=5.6, 2.2 Hz, 1 H), 6.79 (s, 1H), 5.10 (q, J=8.3 Hz, 2H), 2.74-2.81 (m, 2H), 2.54-2.60 (m, 2H), 2.47-2.94 (m, 8H), 2.39 (s, 3H).

### Example 51: N-[6-(5-chloro-2-fluorophenyl)-3-(2,2,2-trifluoroethoxy)pyridazin-4-yl]-7-[2-(4-methylpiperazin-1-yl)ethoxy]quinolin-4-amine

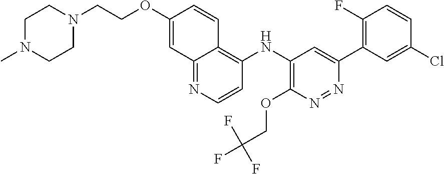 [SMILES: CN1CCN(CCOc2ccc3c(Nc4cc(-c5cc(Cl)ccc5F)nnc4OCC(F)(F)F)ccnc3c2)CC1]

Example 51 was prepared following the procedure used for the synthesis of Example 2, starting from 4-chloro-7-[2-(4-methylpiperazin-1-yl)ethoxy]quinoline (Intermediate 5, 73 mg, 0.24 mmol) and 6-(5-chloro-2-fluorophenyl)-3-(2,2,2-trifluoroethoxy)pyridazin-4-amine (Intermediate 125, 70 mg, 0.22 mmol) to afford title compound (40 mg, 0.07 mmol, 31% yield).

LC-MS (ESI): m/z (M+1): 591.3 (Method 2)

¹H NMR (400 MHz, Chloroform-d) δ ppm 8.83 (d, J=4.9 Hz, 1H), 8.05 (dd, J=6.7, 2.7 Hz, 1H), 7.81 (d, J=9.2 Hz, 1H), 7.65 (d, J=1.6 Hz, 1H), 7.49 (d, J=2.5 Hz, 1H), 7.38 (ddd, J=8.8, 4.2, 2.8 Hz, 1H), 7.34 (d, J=5.0 Hz, 1H), 7.31 (dd, J=9.2, 2.5 Hz, 1H), 7.06-7.16 (m, 2H), 5.16 (q, J=8.2 Hz, 2H), 4.30 (t, J=5.7 Hz, 2H), 2.93 (t, J=5.7 Hz, 2H), 2.40-2.79 (m, 8H), 2.31 (s, 3H).

### Example 52: N-(4-{[6-(5-chloro-2-fluorophenyl)-3-(2,2-difluoroethoxy)pyridazin-4-yl]amino}pyridin-2-yl)-3-(4-methylpiperazin-1-yl)propanamide

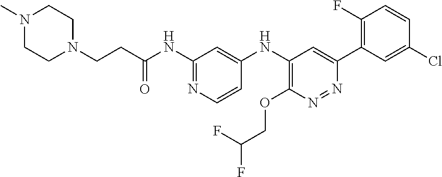 [SMILES: CN1CCN(CCC(=O)Nc2cc(Nc3cc(-c4cc(Cl)ccc4F)nnc3OCC(F)F)ccn2)CC1]

Example 52 was prepared following the procedure used for the synthesis of Example 1, starting from N-(4-bromopyridin-2-yl)-3-(4-methylpiperazin-1-yl)propanamide (Intermediate 2, 71 mg, 0.22 mmol) and 6-(5-chloro-2-fluorophenyl)-3-(2,2-difluoroethoxy)pyridazin-4-amine (Intermediate 9, 60 mg, 0.20 mmol) to afford title compound (80 mg, 0.14 mmol, 74% yield).

LC-MS (ESI): m/z (M+1): 550.5 (Method 2)

¹H NMR (400 MHz, Chloroform-d) δ ppm 11.29 (br. s, 1H), 8.26 (d, J=5.5 Hz, 1H), 8.11 (d, J=2.0 Hz, 1H), 8.08 (dd, J=6.7, 2.7 Hz, 1H), 7.81 (s, 1H), 7.35-7.44 (m, 1H), 7.14 (dd, J=10.4, 8.9 Hz, 1H), 6.95 (dd, J=5.6, 2.1 Hz, 1H), 6.85 (s, 1H), 6.34 (tt, J=55.2, 4.0 Hz, 1H), 4.89 (td, J=13.3, 4.2 Hz, 2H), 2.46-2.86 (m, 12H), 2.37 (s, 3H).

### Example 53: N4-[6-(5-chloro-2-fluorophenyl)-3-(2,2-difluoroethoxy)pyridazin-4-yl]pyridine-2,4-diamine

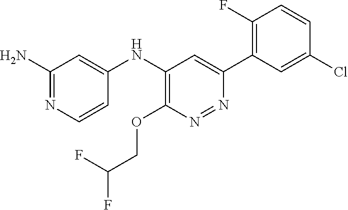 [SMILES: Nc1cc(Nc2cc(-c3cc(Cl)ccc3F)nnc2OCC(F)F)ccn1]

Example 53 was prepared following the procedure used for the synthesis of Example 43, starting from tert-butyl N-(4-{[6-(5-chloro-2-fluorophenyl)-3-(2,2-difluoroethoxy)pyridazin-4-yl]amino}pyridin-2-yl)carbamate (Intermediate 127, 60 mg, 0.12 mmol) to afford title compound (30 mg, 0.08 mmol, 63% yield).

LC-MS (ESI): m/z (M+1): 396.2 (Method 1)

¹H NMR (400 MHz, Chloroform-d) δ ppm 8.00-8.16 (m, 2H), 7.74 (d, J=1.5 Hz, 1H), 7.40 (ddd, J=8.7, 4.3, 2.6 Hz, 1H), 7.14 (dd, J=10.5, 8.8 Hz, 1H), 6.63 (s, 1H), 6.56 (dd, J=5.7, 2.0 Hz, 1H), 6.15-6.50 (m, 2H), 4.88 (td, J=13.5, 3.9 Hz, 2H), 4.47-4.63 (m, 2H).

### Example 54: N-(4-{[6-(5-chloro-2-fluorophenyl)-3-[2-(pyrrolidin-1-yl)ethoxy]pyridazin-4-yl]amino}pyridin-2-yl)cyclopropanecarboxamide

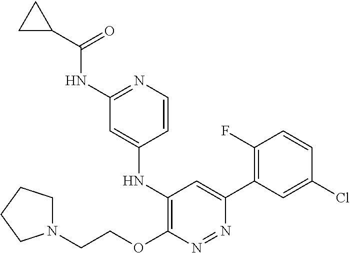 [SMILES: O=C(Nc1cc(Nc2cc(-c3cc(Cl)ccc3F)nnc2OCCN2CCCC2)ccn1)C1CC1]

Example 54 was prepared following the procedure used for the synthesis of Example 1, starting from 6-(5-chloro-2-fluorophenyl)-3-[2-(pyrrolidin-1-yl)ethoxy]pyridazin-4-amine (Intermediate 130, 55 mg, 0.16 mmol) and N-(4-bromopyridin-2-yl)cyclopropanecarboxamide (Intermediate 112, 43 mg, 0.18 mmol) to afford title compound (53 mg, 0.11 mmol, 65% yield).

LC-MS (ESI): m/z (M+1): 497.3 (Method 2)

¹H NMR (400 MHz, Chloroform-d) δ ppm 8.65 (s, 1H), 8.20 (s, 1H), 8.16 (d, J=5.7 Hz, 1H), 8.11 (d, J=2.0 Hz, 1H), 8.09 (dd, J=6.7, 2.7 Hz, 1H), 7.76 (d, J=1.5 Hz, 1H), 7.36 (ddd, J=8.8, 4.2, 2.9 Hz, 1H), 7.12 (dd, J=10.5, 8.8 Hz, 1H), 7.01 (dd, J=5.7, 2.0 Hz, 1H), 4.68-4.81 (m, 2H), 2.96-3.08 (m, 2H), 2.62-2.77 (m, 4H), 1.86 (br. t, J=3.3 Hz, 4H), 1.50-1.65 (m, 1H), 1.06-1.17 (m, 2H), 0.85-0.93 (m, 2H).

### Example 55: N4-[6-(5-chloro-2-fluorophenyl)-3-[3-(methylsulfanyl)propoxy]pyridazin-4-yl]pyridine-2,4-diamine

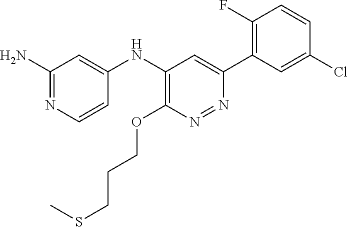 [SMILES: CSCCCOc1nnc(-c2cc(Cl)ccc2F)cc1Nc1ccnc(N)c1]

Example 55 was prepared following the procedure used for the synthesis of Example 43, starting from tert-butyl N-(4-{[6-(5-chloro-2-fluorophenyl)-3-[3-(methylsulfanyl)propoxy]pyridazin-4-yl]amino}pyridin-2-yl)carbamate (Intermediate 131, 50 mg, 0.10 mmol) to afford title compound (19 mg, 0.04 mmol, 47% yield).

LC-MS (ESI): m/z (M+1): 420.5 (Method 2)

¹H NMR (600 MHz, Chloroform-d) δ ppm 8.09 (dd, J=6.7, 2.7 Hz, 1H), 8.03 (d, J=5.8 Hz, 1H), 7.70 (d, J=1.6 Hz, 1H), 7.38 (ddd, J=8.7, 4.3, 2.8 Hz, 1H), 7.13 (dd, J=10.6, 8.8 Hz, 1H), 6.86 (s, 1H), 6.54 (dd, J=5.6, 2.0 Hz, 1H), 6.35 (d, J=1.8 Hz, 1H), 4.78 (t, J=6.3 Hz, 2H), 4.58 (br. s, 2H), 2.74 (t, J=6.8 Hz, 2H), 2.26 (quin, J=6.6 Hz, 2H), 2.18 (s, 3H).

### Example 56: N4-[6-(5-chloro-2-fluorophenyl)-3-(3-methanesulfonylpropoxy)pyridazin-4-yl]pyridine-2,4-diamine

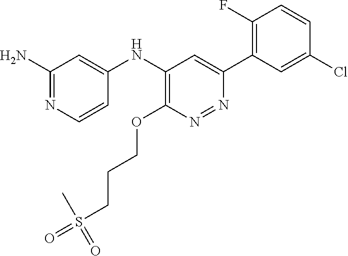 [SMILES: CS(=O)(=O)CCCOc1nnc(-c2cc(Cl)ccc2F)cc1Nc1ccnc(N)c1]

Example 56 was prepared following the procedure used for the synthesis of Example 43, starting from tert-butyl N-(4-{[6-(5-chloro-2-fluorophenyl)-3-(3-methanesulfonylpropoxy)pyridazin-4-yl]amino}pyridin-2-yl)carbamate (Intermediate 132, 70 mg, 0.13 mmol) to afford title compound (51 mg, 0.11 mmol, 89% yield).

LC-MS (ESI): m/z (M+1): 452.1 (Method 1)

¹H NMR (400 MHz, DMSO-d₆) δ ppm 8.51 (s, 1H), 7.90 (dd, J=6.6, 2.6 Hz, 1H), 7.83 (d, J=5.7 Hz, 1H), 7.55-7.61 (m, 1H), 7.54 (d, J=1.5 Hz, 1H), 7.42 (dd, J=10.5, 8.8 Hz, 1H), 6.50 (dd, J=5.7, 2.0 Hz, 1H), 6.37 (d, J=2.0 Hz, 1H), 5.89 (s, 2H), 4.64 (t, J=6.1 Hz, 2H), 3.40-3.55 (m, 2H), 3.03 (s, 3H), 2.18-2.38 (m, 2H).

### Example 57: N4-[6-(5-chloro-2-fluorophenyl)-3-(3-methanesulfinylpropoxy)pyridazin-4-yl]pyridine-2,4-diamine

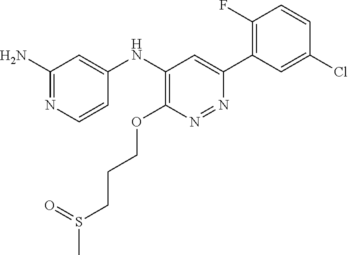 [SMILES: CS(=O)CCCOc1nnc(-c2cc(Cl)ccc2F)cc1Nc1ccnc(N)c1]

Example 57 was prepared following the procedure used for the synthesis of Example 43, starting from tert-butyl N-(4-{[6-(5-chloro-2-fluorophenyl)-3-(3-methanesulfinylpropoxy)pyridazin-4-yl]amino}pyridin-2-yl)carbamate (Intermediate 133, 75 mg, 0.14 mmol) to afford title compound (39 mg, 0.09 mmol, 64% yield).

LC-MS (ESI): m/z (M+1): 436.1 (Method 1)

¹H NMR (400 MHz, DMSO-d₆) δ ppm 8.61 (s, 1H), 7.90 (dd, J=6.7, 2.7 Hz, 1H), 7.82 (d, J=5.7 Hz, 1H), 7.51-7.62 (m, 2H), 7.42 (dd, J=10.4, 8.9 Hz, 1H), 6.50 (dd, J=5.6, 1.9 Hz, 1H), 6.37 (d, J=1.8 Hz, 1H), 5.87 (s, 2H), 4.64 (t, J=6.1 Hz, 2H), 2.98-3.13 (m, 1H), 2.93 (dt, J=13.5, 6.9 Hz, 1H), 2.59 (s, 3H), 2.15-2.32 (m, 2H).

### Example 58: (3-{[6-(5-chloro-2-fluorophenyl)-4-[(2-cyclopropaneamidopyridin-4-yl)amino]pyridazin-3-yl]oxy}propyl)trimethylazanium chloride

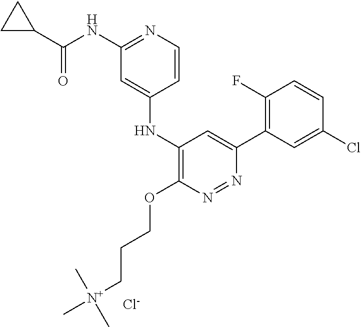 [SMILES: C[N+](C)(C)CCCOc1nnc(-c2cc(Cl)ccc2F)cc1Nc1ccnc(NC(=O)C2CC2)c1 ; [Cl-]]

Example 58 was prepared following the procedure used for the synthesis of Example 11, starting from N-(4-{[6-(5-chloro-2-fluorophenyl)-3-[3-(dimethylamino)propoxy]pyridazin-4-yl]amino}pyridin-2-yl)cyclopropanecarboxamide (Intermediate 134, 65 mg, 0.13 mmol) to afford title compound (18 mg, 0.03 mmol, 26% yield).

LC-MS (ESI): m/z (M+1): 499.4 (Method 1)

¹H NMR (400 MHz, DMSO-d₆) δ ppm 11.58-12.76 (m, 1H), 9.88-10.97 (m, 1H), 8.12 (d, J=6.6 Hz, 1H), 7.96 (dd, J=6.4, 2.9 Hz, 1H), 7.93 (s, 1H), 7.59-7.68 (m, 1H), 7.39-7.58 (m, 2H), 7.31 (br. d, J=5.3 Hz, 1H), 4.67 (t, J=5.7 Hz, 2H), 3.26-4.21 (m, 2H), 3.12 (s, 9H), 2.21-2.40 (m, 2H), 1.95-2.06 (m, 1H), 0.87-1.04 (m, 4H).

### Example 59: N-(4-{[6-(5-chloro-2-fluorophenyl)-3-(oxolan-3-yloxy)pyridazin-4-yl]amino}pyridin-2-yl)-3-(4-methylpiperazin-1-yl)propanamide

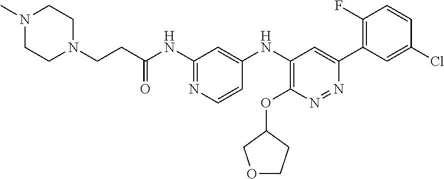 [SMILES: CN1CCN(CCC(=O)Nc2cc(Nc3cc(-c4cc(Cl)ccc4F)nnc3OC3CCOC3)ccn2)CC1]

Example 59 was prepared following the procedure used for the synthesis of Example 1, starting from 6-(5-chloro-2-fluorophenyl)-3-(oxolan-3-yloxy)pyridazin-4-amine (Intermediate 136, 100 mg, 0.32 mmol) and N-(4-bromopyridin-2-yl)-3-(4-methylpiperazin-1-yl)propanamide (Intermediate 2, 116 mg, 0.36 mmol) to afford title compound (89 mg, 0.16 mmol, 50% yield).

LC-MS (ESI): m/z (M+1): 556.3 (Method 2)

¹H NMR (400 MHz, Chloroform-d) δ ppm 11.10-11.37 (m, 1H), 8.25 (d, J=5.7 Hz, 1H), 8.11 (dd, J=6.6, 2.6 Hz, 1H), 8.08 (d, J=2.0 Hz, 1H), 7.78 (d, J=1.1 Hz, 1H), 7.37 (ddd, J=8.7, 4.1, 2.9 Hz, 1H), 7.13 (dd, J=10.5, 9.0 Hz, 1H), 6.96 (dd, J=5.6, 2.1 Hz, 1H), 6.88 (s, 1H), 5.94 (dt, J=4.2, 2.1 Hz, 1H), 4.06-4.25 (m, 3H), 3.96 (td, J=8.4, 5.0 Hz, 1H), 2.73-2.80 (m, 1H), 2.53-2.60 (m, 2H), 2.44-2.85 (m, 8H), 2.47 (td, J=14.3, 8.0 Hz, 1H), 2.29-2.41 (m, 4H).

### Example 60: N-(4-{[6-(5-chloro-2-fluorophenyl)-3-[(2-hydroxyethyl)sulfanyl]pyridazin-4-yl]amino}pyridin-2-yl)-2-(piperazin-1-yl)acetamide

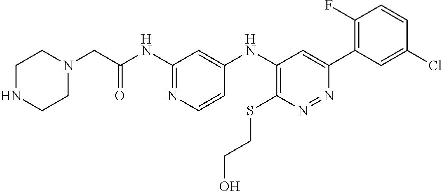 [SMILES: O=C(CN1CCNCC1)Nc1cc(Nc2cc(-c3cc(Cl)ccc3F)nnc2SCCO)ccn1]

Example 60 was prepared following the procedure used for the synthesis of Example 23, starting from tert-butyl 4-{[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]methyl}piperazine-1-carboxylate (Intermediate 137, 130 mg, 0.18 mmol) to afford title compound (66 mg, 0.13 mmol, 72% yield).

LC-MS (ESI): m/z (M+1): 518.2 (Method 2)

¹H NMR (500 MHz, Chloroform-d) δ ppm 9.67 (s, 1H), 8.26 (d, J=5.6 Hz, 1H), 8.14 (dd, J=6.7, 2.7 Hz, 1H), 8.11 (d, J=2.1 Hz, 1H), 7.75 (d, J=1.4 Hz, 1H), 7.35-7.44 (m, 1H), 7.14 (dd, J=10.6, 8.8 Hz, 1H), 6.96 (dd, J=5.7, 2.1 Hz, 1H), 6.54 (s, 1H), 4.07 (t, J=5.6 Hz, 2H), 3.67 (t, J=5.6 Hz, 2H), 3.17 (s, 2H), 3.07-3.46 (m, 1H), 3.00 (t, J=4.9 Hz, 4H), 2.60 (br. s, 4H).

### Example 61: N-(4-{[6-(5-chloro-2-fluorophenyl)-3-[(2-hydroxyethyl)sulfanyl]pyridazin-4-yl]amino}pyridin-2-yl)-2-(1,4-diazepan-1-yl)acetamide

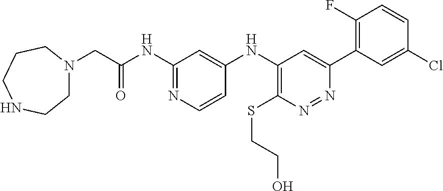 [SMILES: O=C(CN1CCCNCC1)Nc1cc(Nc2cc(-c3cc(Cl)ccc3F)nnc2SCCO)ccn1]

Example 61 was prepared following the procedure used for the synthesis of Example 23, starting from tert-butyl 4-{[(4-{[3-({2-[(tert-butyldimethylsilyl)oxy]ethyl}sulfanyl)-6-(5-chloro-2-fluorophenyl)pyridazin-4-yl]amino}pyridin-2-yl)carbamoyl]methyl}-1,4-diazepane-1-carboxylate (Intermediate 139, 105 mg, 0.14 mmol) to afford title compound (45 mg, 0.08 mmol, 60% yield).

LC-MS (ESI): m/z (M+1): 532.2 (Method 2)

¹H NMR (500 MHz, Chloroform-d) 9.83 (s, 1H), 8.26 (d, J=5.6 Hz, 1H), 8.15 (dd, J=6.7, 2.7 Hz, 1H), 8.12 (d, J=2.1 Hz, 1H), 7.75 (d, J=1.2 Hz, 1H), 7.40 (ddd, J=8.8, 4.3, 2.7 Hz, 1H), 7.14 (dd, J=10.6, 8.8 Hz, 1H), 6.95 (dd, J=5.6, 2.2 Hz, 1H), 6.53 (s, 1H), 4.08 (t, J=5.6 Hz, 2H), 3.62-3.73 (m, 2H), 3.35 (s, 1H), 3.29 (br. s, 1H), 3.03 (t, J=6.2 Hz, 2H), 2.98-3.01 (m, 2H), 2.88-2.92 (m, 2H), 2.83-2.87 (m, 2H), 1.87 (quin, J=6.0 Hz, 2H).

### Example 62: N-(4-{[6-(5-chloro-2-fluorophenyl)-3-(methylsulfanyl)pyridazin-4-yl]amino}pyridin-2-yl)-3-(piperazin-1-yl)propanamide

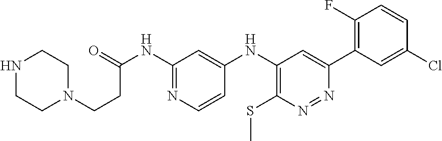 [SMILES: CSc1nnc(-c2cc(Cl)ccc2F)cc1Nc1ccnc(NC(=O)CCN2CCNCC2)c1]

Example 62 was prepared following the procedure used for the synthesis of Example 43, starting from tert-butyl 4-{2-[(4-{[6-(5-chloro-2-fluorophenyl)-3-(methylsulfanyl)pyridazin-4-yl]amino}pyridin-2-yl)carbamoyl]ethyl}piperazine-1-carboxylate (Intermediate 140, 110 mg, 0.18 mmol) to afford title compound (34 mg, 0.07 mmol, 37% yield).

LC-MS (ESI): m/z (M+1): 502.2 (Method 2)

¹H NMR (400 MHz, Chloroform-d) δ ppm 11.40 (s, 1H), 8.15-8.26 (m, 2H), 8.04 (d, J=2.0 Hz, 1H), 7.72 (d, J=1.3 Hz, 1H), 7.38 (ddd, J=8.8, 4.3, 2.7 Hz, 1H), 7.13 (dd, J=10.7, 8.8 Hz, 1H), 6.91 (dd, J=5.7, 2.2 Hz, 1H), 6.32 (s, 1H), 3.06 (t, J=4.7 Hz, 4H), 2.88 (s, 3H), 2.72-2.79 (m, 2H), 2.49-2.70 (m, 6H).

### Example 63: N-(4-{[6-(5-chloro-2-fluorophenyl)-3-[(2-hydroxyethyl)sulfanyl]pyridazin-4-yl]amino}pyridin-2-yl)-2-[(1R,4R)-5-methyl-2,5-diazabicyclo[2.2.1]heptan-2-yl]acetamide

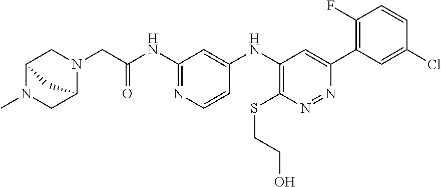 [SMILES: CN1C[C@H]2C[C@@H]1CN2CC(=O)Nc1cc(Nc2cc(-c3cc(Cl)ccc3F)nnc2SCCO)ccn1]

Example 63 was prepared following the procedure used for the synthesis of Example 23, starting from N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-[(1R,4R)-5-methyl-2,5-diazabicyclo[2.2.1]heptan-2-yl]acetamide (Intermediate 142, 55 mg, 0.08 mmol) to afford title compound (38 mg, 0.07 mmol, 85% yield).

LC-MS (ESI): m/z (M+1): 544.3 (Method 2)

¹H NMR (500 MHz, Chloroform-d) 9.74 (s, 1H), 8.25 (d, J=5.7 Hz, 1H), 8.15 (dd, J=6.7, 2.7 Hz, 1H), 8.12 (d, J=2.0 Hz, 1H), 7.75 (s, 1H), 7.40 (ddd, J=8.7, 4.2, 2.8 Hz, 1H), 7.14 (dd, J=10.4, 8.9 Hz, 1H), 6.95 (dd, J=5.7, 2.2 Hz, 1H), 6.54 (s, 1H), 4.08 (t, J=5.5 Hz, 2H), 3.67 (t, J=5.5 Hz, 2H), 3.31-3.45 (m, 3H), 3.30 (s, 1H), 3.21-3.45 (m, 1H), 2.84-2.95 (m, 2H), 2.68-2.84 (m, 2H), 2.42 (s, 3H), 1.74-1.94 (m, 2H).

### Example 64: N-(4-{[6-(5-chloro-2-fluorophenyl)-3-[(2-hydroxyethyl)sulfanyl]pyridazin-4-yl]amino}pyridin-2-yl)-2-[(1S,4S)-5-methyl-2,5-diazabicyclo[2.2.1]heptan-2-yl]acetamide

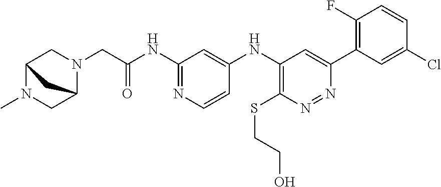 [SMILES: CN1C[C@@H]2C[C@H]1CN2CC(=O)Nc1cc(Nc2cc(-c3cc(Cl)ccc3F)nnc2SCCO)ccn1]

Example 64 was prepared following the procedure used for the synthesis of Example 23, starting from N-(4-{[3-({2-[(tert-butyldimethylsilyl)oxy]ethyl}sulfanyl)-6-(5-chloro-2-fluorophenyl)pyridazin-4-yl]amino}pyridin-2-yl)-2-[(1S,4S)-5-methyl-2,5-diazabicyclo[2.2.1]heptan-2-yl]acetamide (Intermediate 144, 90 mg, 0.14 mmol) to afford title compound (43 mg, 0.08 mmol, 57% yield).

LC-MS (ESI): m/z (M+1): 544.3 (Method 2)

¹H NMR (500 MHz, Chloroform-d) 9.74 (s, 1H), 8.25 (d, J=5.7 Hz, 1H), 8.15 (dd, J=6.7, 2.7 Hz, 1H), 8.12 (d, J=2.0 Hz, 1H), 7.75 (s, 1H), 7.40 (ddd, J=8.7, 4.2, 2.8 Hz, 1H), 7.14 (dd, J=10.4, 8.9 Hz, 1H), 6.95 (dd, J=5.7, 2.2 Hz, 1H), 6.54 (s, 1H), 4.08 (t, J=5.5 Hz, 2H), 3.67 (t, J=5.5 Hz, 2H), 3.31-3.45 (m, 3H), 3.30 (s, 1H), 3.21-3.45 (m, 1H), 2.84-2.95 (m, 2H), 2.68-2.84 (m, 2H), 2.42 (s, 3H), 1.74-1.94 (m, 2H).

### Example 65: N-(4-{[6-(5-chloro-2-fluorophenyl)-3-[(2-hydroxyethyl)(methyl)amino]pyridazin-4-yl]amino}pyridin-2-yl)-3-(4-methylpiperazin-1-yl)propanamide

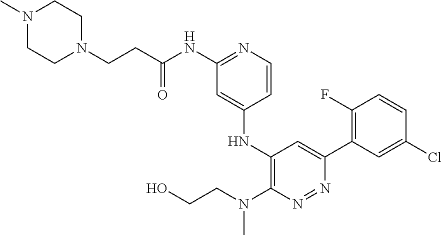 [SMILES: CN1CCN(CCC(=O)Nc2cc(Nc3cc(-c4cc(Cl)ccc4F)nnc3N(C)CCO)ccn2)CC1]

Example 65 was prepared following the procedure used for the synthesis of Example 1, starting from 2-{[4-amino-6-(5-chloro-2-fluorophenyl)pyridazin-3-yl](methyl)amino}ethan-1-ol (Intermediate 145, 30 mg, 0.10 mmol) and N-(4-bromopyridin-2-yl)-3-(4-methylpiperazin-1-yl)propanamide (Intermediate 2, 41 mg, 0.13 mmol) to afford title compound (6.5 mg, 0.01 mmol, 12% yield).

LC-MS (ESI): m/z (M+1): 543.3 (Method 2)

¹H NMR (500 MHz, Chloroform-d) δ ppm 11.30 (s, 1H), 8.15 (d, J=2.3 Hz, 1H), 8.14 (d, J=5.9 Hz, 1H), 7.62 (dd, J=6.2, 2.6 Hz, 1H), 7.44 (ddd, J=8.8, 4.4, 2.7 Hz, 1H), 7.16 (t, J=9.1 Hz, 1H), 7.06 (s, 1H), 6.55 (dd, J=5.6, 2.2 Hz, 1H), 6.02 (s, 1H), 4.38 (br. s, 1H), 3.86-3.95 (m, 2H), 3.72-3.84 (m, 2H), 3.26 (s, 3H), 2.73-2.77 (m, 2H), 2.53-2.56 (m, 2H), 2.41-2.89 (m, 8H), 2.36 (s, 3H).

### Example 66: N-(4-{[6-(5-chloro-2-fluorophenyl)-3-(oxetan-3-yloxy)pyridazin-4-yl]amino}pyridin-2-yl)-3-(4-methylpiperazin-1-yl)propanamide

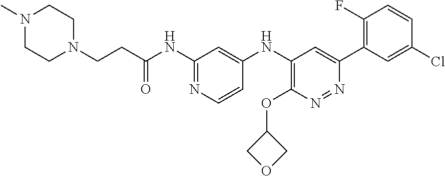 [SMILES: CN1CCN(CCC(=O)Nc2cc(Nc3cc(-c4cc(Cl)ccc4F)nnc3OC3COC3)ccn2)CC1]

Example 66 was prepared following the procedure used for the synthesis of Example 1, starting from 6-(5-chloro-2-fluorophenyl)-3-(oxetan-3-yloxy)pyridazin-4-amine (Intermediate 146, 34 mg, 0.11 mmol) and N-(4-bromopyridin-2-yl)-3-(4-methylpiperazin-1-yl)propanamide (Intermediate 2, 47 mg, 0.14 mmol) to afford title compound (13 mg, 0.02 mmol, 21% yield).

LC-MS (ESI): m/z (M+1): 542.3 (Method 2)

¹H NMR (500 MHz, Chloroform-d) δ ppm 11.28 (s, 1H), 8.27 (d, J=5.6 Hz, 1H), 8.11 (d, J=2.0 Hz, 1H), 8.09 (dd, J=6.7, 2.7 Hz, 1H), 7.81 (d, J=1.2 Hz, 1H), 7.38 (ddd, J=8.7, 4.2, 2.8 Hz, 1H), 7.13 (dd, J=10.5, 8.8 Hz, 1H), 6.98 (dd, J=5.6, 2.1 Hz, 1H), 6.89 (s, 1H), 5.94 (quin, J=5.7 Hz, 1H), 5.16 (t, J=7.1 Hz, 2H), 4.91 (dd, J=7.8, 5.3 Hz, 2H), 2.75-2.81 (m, 2H), 2.54-2.60 (m, 2H), 2.62 (br. s, 8H), 2.37 (s, 3H).

### Example 67: N-(4-{[6-(5-chloro-2-fluorophenyl)-3-(oxetan-3-yloxy)pyridazin-4-yl]amino}pyridin-2-yl)-2-(4-methyl-1,4-diazepan-1-yl)acetamide

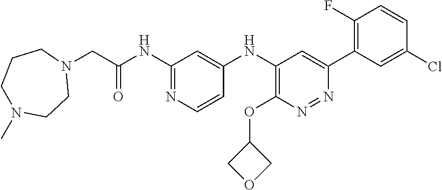 [SMILES: CN1CCCN(CC(=O)Nc2cc(Nc3cc(-c4cc(Cl)ccc4F)nnc3OC3COC3)ccn2)CC1]

Example 66 was prepared following the procedure used for the synthesis of Example 1, starting from 6-(5-chloro-2-fluorophenyl)-3-(oxetan-3-yloxy)pyridazin-4-amine (Intermediate 146, 40 mg, 0.13 mmol) and N-(4-bromopyridin-2-yl)-2-(4-methyl-1,4-diazepan-1-yl)acetamide (Intermediate 82, 56 mg, 0.16 mmol) to afford title compound (20 mg, 0.04 mmol, 27% yield).

LC-MS (ESI): m/z (M+1): 542.3 (Method 2)

¹H NMR (500 MHz, Chloroform-d) δ ppm 9.81 (s, 1H), 8.29 (d, J=5.6 Hz, 1H), 8.17 (d, J=2.1 Hz, 1H), 8.09 (dd, J=6.7, 2.7 Hz, 1H), 7.83 (d, J=1.2 Hz, 1H), 7.38 (ddd, J=8.7, 4.2, 2.7 Hz, 1H), 7.14 (dd, J=10.6, 8.8 Hz, 1H), 7.02 (dd, J=5.7, 2.1 Hz, 1H), 6.92 (s, 1H), 5.90-5.98 (m, 1H), 5.16 (t, J=7.3 Hz, 2H), 4.91 (dd, J=8.5, 5.2 Hz, 2H), 3.33 (s, 2H), 2.84-2.96 (m, 4H), 2.66-2.77 (m, 4H), 2.42 (s, 3H), 1.91 (quin, J=5.9 Hz, 2H).

### Example 68: N-(4-{[6-(3-fluoro-6-methylpyridin-2-yl)-3-(2,2,2-trifluoroethoxy)pyridazin-4-yl]amino}pyridin-2-yl)-3-(4-methylpiperazin-1-yl)propanamide

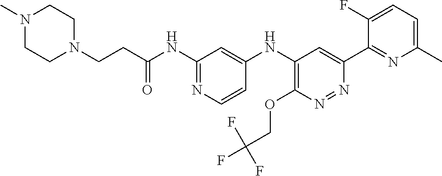

### Step 1

2-bromo-3-fluoro-6-methylpyridine (100 mg, 0.53 mmol) and hexamethyldistannane (172 mg, 0.53 mmol) were mixed in 1,4-dioxane (1 mL), N₂ was bubbled for 5 min before adding PdCl₂(PPh₃)₂ (37 mg, 0.05 mmol), the vial was closed and heated at 80° C. for 1.5 hrs. The cooled mixture was diluted with EtOAc and brine, the organic phase was separated, filtered over a phase separator, and evaporated to afford a residue containing 22% a/a of 3-fluoro-6-methyl-2-(trimethylstannyl)pyridine (380 mg,), that was used as such.

### Step 2

Copper (I) iodide (6.4 mg, 0.03 mmol), N-(4-{[6-chloro-3-(2,2,2-trifluoroethoxy)pyridazin-4-yl]amino}pyridin-2-yl)-3-(4-methylpiperazin-1-yl)propanamide (Intermediate 148, 80 mg, 0.17 mmol) and 3-fluoro-6-methyl-2-(trimethylstannyl)pyridine (380 mg, from previous step) were mixed in DMF (1.1 mL). After bubbling N₂ for 5 min Pd(dppf)Cl₂ (6 mg, 0.01 mmol) was added, and the mixture was heated at 100° C. for 1 h. The mixture cooled to RT, charged on SCX, washing with MeOH, and eluting with 1 N NH₃ in MeOH. Basic fractions were collected and evaporated, the residual material was purified by reverse flash chromatography on Biotage C18 cartridge (from H₂O+0.1% HCOOH to 30% HCOOH), then further purified by HPLC affording title compound (11 mg, 0.02 mmol, 12% yield).

LC-MS (ESI): m/z (M+1): 549.4 (Method 1)

¹H NMR (500 MHz, Chloroform-d) δ ppm 11.21 (br. s, 1H), 8.26 (d, J=5.6 Hz, 1H), 8.14 (s, 2H), 7.43-7.52 (m, 1H), 7.23 (dd, J=8.5, 3.3 Hz, 1H), 6.99 (dd, J=5.6, 2.1 Hz, 1H), 6.81 (s, 1H), 5.11 (q, J=8.2 Hz, 2H), 2.77 (br. t, J=5.8 Hz, 2H), 2.63 (s, 3H), 2.54-2.59 (m, 2H), 2.46-3.02 (m, 8H), 2.37 (s, 3H).

### Example 69: N4-[6-(5-chloro-2-fluorophenyl)-3-(2-methoxyethoxy)pyridazin-4-yl]pyridine-2,4-diamine

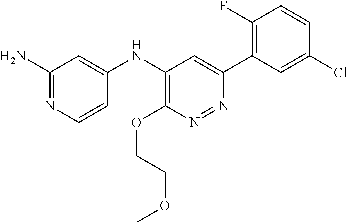 [SMILES: COCCOc1nnc(-c2cc(Cl)ccc2F)cc1Nc1ccnc(N)c1]

Example 69 was prepared following the procedure used for the synthesis of Intermediate 8, starting from Intermediate 150 (30 mg, 0.076 mmol) and using (5-chloro-2-fluorophenyl)boronic acid (20 mg, 0.114 mmol). Purification by reverse flash chromatography (from 100% H₂O/MeCN 95:5+0.1% HCOOH to 40% MeCN/H₂O 95:5 +0.1% HCOOH) afforded the title compound (5 mg, 0.013 mmol, 17% yield).

LC-MS (ESI): m/z (M+1): 390.3 (Method 2)

¹H NMR (600 MHz, DMSO-d6) δ ppm 8.57 (br s, 1H) 7.91 (dd, J=6.60, 2.76 Hz, 1H) 7.81 (d, J=5.64 Hz, 1H) 7.58 (ddd, J=6.60, 4.30, 2.05 Hz, 1H) 7.56 (d, J=1.41 Hz, 1H) 7.42 (dd, J=10.51, 8.85 Hz, 1H) 6.52 (dd, J=5.64, 2.05 Hz, 1H) 6.39 (d, J=1.79 Hz, 1H) 5.88 (s, 2H) 4.69 (dd, J=5.32, 4.17 Hz, 2H) 3.78-3.84 (m, 2H).

### Example 70: N4-[6-(5-chloro-2-fluorophenyl)-3-[2-(4-methylpiperazin-1-yl)ethoxy]pyridazin-4-yl]pyridine-2,4-diamine

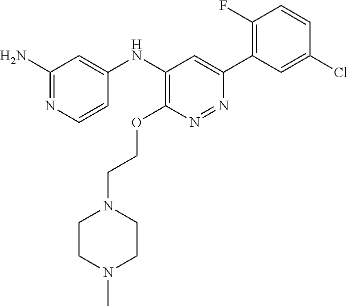 [SMILES: CN1CCN(CCOc2nnc(-c3cc(Cl)ccc3F)cc2Nc2ccnc(N)c2)CC1]

Example 70 was prepared following the procedure used for the synthesis of Intermediate 8, starting from Intermediate 152 (268 mg, 0.737 mmol) and using (5-chloro-2-fluorophenyl)boronic acid (193 mg, 1.105 mmol). Purification by reverse flash chromatography (from 100% H₂O/MeCN 95:5+0.1% HCOOH to 40% MeCN/H₂O 95:5 +0.1% HCOOH) afforded the title compound (5 mg, 10.92 μmol, 1.5% yield).

LC-MS (ESI): m/z (M+1): 458.3 (Method 2)

¹H NMR (600 MHz, DMSO-d6) δ ppm 8.23 (s, 2H) 7.90 (dd, J=6.53, 2.80 Hz, 1H) 7.81 (d, J=5.69 Hz, 1H) 7.58 (ddd, J=8.85, 4.18, 2.70 Hz, 1H) 7.55 (d, J=1.52 Hz, 1H) 7.39-7.44 (m, 1H) 6.50 (dd, J=5.70, 1.99 Hz, 1H) 6.37 (d, J=2.00 Hz, 1H) 4.66 (t, J=6.15 Hz, 2H) 2.84 (t, J=6.11 Hz, 2H) 2.31 (br s, 4H) 2.14 (s, 3H).

### Example 71: Cis N-(4-{[6-(5-chloro-2-fluorophenyl)-3-[(3-hydroxycyclobutyl)methoxy]pyridazin-4-yl]amino}pyridin-2-yl)-3-(4-methylpiperazin-1-yl)propanamide

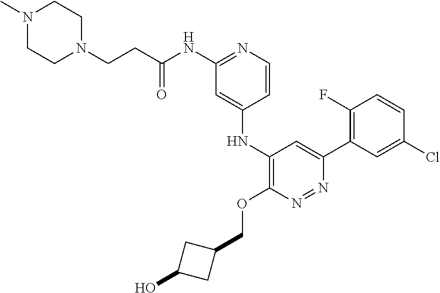

Example 71 was prepared following the procedure used for the synthesis of Example 1, starting from 3-({[4-amino-6-(5-chloro-2-fluorophenyl)pyridazin-3-yl]oxy}methyl)cyclobutan-1-ol (Intermediate 157, 66 mg, 0.20 mmol) and N-(4-bromopyridin-2-yl)-3-(4-methylpiperazin-1-yl)propanamide (Intermediate 2, 73 mg, 0.22 mmol) to afford title compound (65 mg, 0.11 mmol, 56% yield).

LC-MS (ESI): m/z (M+1): 570.5 (Method 1)

¹H NMR (400 MHz, Chloroform-d) δ ppm 11.17 (br s, 1H), 8.23 (d, J=5.6 Hz, 1H), 8.10 (dd, J=6.6, 2.5 Hz, 1H), 8.05 (s, 1H), 7.76 (s, 1H), 7.51 (s, 1H), 7.33-7.41 (m, 1H), 7.13 (dd, J=10.2, 9.1 Hz, 1H), 7.00 (br d, J=3.7 Hz, 1H), 4.63 (d, J=4.8 Hz, 2H), 4.38 (quin, J=6.8 Hz, 1H), 2.46-2.87 (m, 15H), 2.37 (s, 3H), 1.93-2.04 (m, 2H).

### Example 72: N-(4-{[6-(5-chloro-2-fluorophenyl)-3-(oxolan-3-yloxy)pyridazin-4-yl]amino}pyridin-2-yl)-2-(4-methyl-1,4-diazepan-1-yl)acetamide

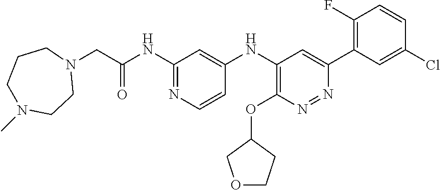 [SMILES: CN1CCCN(CC(=O)Nc2cc(Nc3cc(-c4cc(Cl)ccc4F)nnc3OC3CCOC3)ccn2)CC1]

Example 72 was prepared following the procedure used for the synthesis of Example 1, starting from 6-(5-chloro-2-fluorophenyl)-3-(oxolan-3-yloxy)pyridazin-4-amine (Intermediate 136, 100 mg, 0.32 mmol) and N-(4-bromopyridin-2-yl)-2-(4-methyl-1,4-diazepan-1-yl)acetamide (Intermediate 82, 116 mg, 0.36 mmol) to afford title compound (75 mg, 0.13 mmol, 42% yield).

LC-MS (ESI): m/z (M+1): 556.4 (Method 2)

¹H NMR (400 MHz, Chloroform-d) δ ppm 9.78 (s, 1H), 8.27 (d, J=5.5 Hz, 1H), 8.14 (d, J=1.8 Hz, 1H), 8.11 (dd, J=6.8, 2.6 Hz, 1H), 7.80 (s, 1H), 7.34-7.43 (m, 1H), 7.10-7.18 (m, 1H), 7.00 (dd, J=5.5, 1.8 Hz, 1H), 6.91 (s, 1H), 5.89-6.00 (m, 1H), 4.07-4.22 (m, 3H), 3.96 (td, J=8.4, 5.0 Hz, 1H), 3.33 (s, 2H), 2.84-2.95 (m, 4H), 2.65-2.77 (m, 4H), 2.42-2.55 (m, 1H), 2.41 (s, 3H), 2.26-2.39 (m, 1H), 1.90 (quin, J=5.9 Hz, 2H)

### Example 73 (Enantiomer 1) and Example 74 (Enantiomer 2): N-(4-{[6-(5-chloro-2-fluorophenyl)-3-(oxolan-3-yloxy)pyridazin-4-yl]amino}pyridin-2-yl)-2-(4-methyl-1,4-diazepan-1-yl)acetamide (single enantiomers)

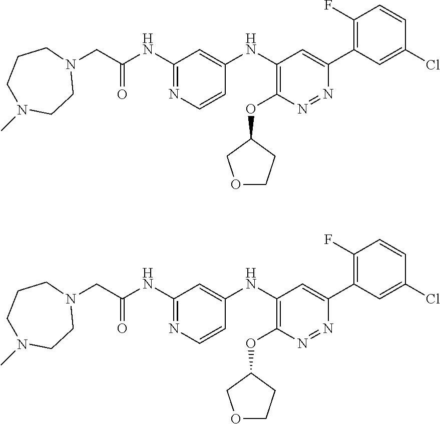 [SMILES: CN1CCCN(CC(=O)Nc2cc(Nc3cc(-c4cc(Cl)ccc4F)nnc3O[C@H]3CCOC3)ccn2)CC1 ; CN1CCCN(CC(=O)Nc2cc(Nc3cc(-c4cc(Cl)ccc4F)nnc3O[C@@H]3CCOC3)ccn2)CC1]

Racemate N-(4-{[6-(5-chloro-2-fluorophenyl)-3-(oxolan-3-yloxy)pyridazin-4-yl]amino}pyridin-2-yl)-2-(4-methyl-1,4-diazepan-1-yl)acetamide (Example 72, 65 mg) was separated into the single enantiomers by preparative chiral HPLC.

### Conditions:

| Column | Chiralpak AS-H (25 × 2.0 cm), 5 μ | |
|---|---|---|
| Mobile phase | n-Hexane/(Ethanol/Methanol 1/1 + 0.1% | |
| | isopropylamine) 80/20% v/v | |
| Flow rate (mL/min) | 16 | mL/min |
| DAD detection | 220 | nm |
| Loop | 600 | μL |

Example 73 was obtained as first eluted enantiomer (24 mg)
Rt.=7.9 min, ee 100%
LC-MS (ESI): m/z (M+1): 556.3 (Method 2)

Example 74 was obtained as the second eluted enantiomer (26 mg)
Rt.=9.7 min, ee 97.8%
LC-MS (ESI): m/z (M+1): 556.3 (Method 2)

### Example 75 (Enantiomer 1) and Example 76 (Enantiomer 2): N-(4-{[6-(5-chloro-2-fluorophenyl)-3-(oxolan-3-yloxy)pyridazin-4-yl]amino}pyridin-2-yl)-3-(4-methylpiperazin-1-yl)propanamide (single enantiomers)

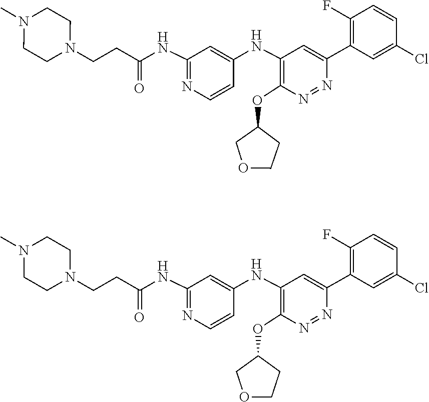

Racemate N-(4-{[6-(5-chloro-2-fluorophenyl)-3-(oxolan-3-yloxy)pyridazin-4-yl]amino}pyridin-2-yl)-3-(4-methylpiperazin-1-yl)propanamide (Example 59, 69 mg) was separated into the single enantiomers by preparative chiral HPLC.

### Conditions:

| Column | Chiralcel OJ-H (25 × 2.0 cm), 5 μ | |
|---|---|---|
| Mobile phase | n-Hexane/(Ethanol + 0.1% | |
| | isopropylamine) 80/20% v/v | |
| Flow rate (mL/min) | 17 | mL/min |
| DAD detection | 220 | nm |
| Loop | 750 | μL |

Example 75 was obtained as first eluted enantiomer (27.8 mg)
Rt.=12.9 min, ee 100%
LC-MS (ESI): m/z (M+1): 556.3 (Method 2)

Example 76 was obtained as second eluted enantiomer (28 mg)
Rt.=18 min, ee 100%
LC-MS (ESI): m/z (M+1): 556.3 (Method 2)

### Example 77: N-(4-{[6-(5-chloro-2-fluorophenyl)-3-[(2,2-dimethyl-1,3-dioxolan-4-yl)methoxy]pyridazin-4-yl]amino}pyridin-2-yl)-3-(4-methylpiperazin-1-yl)propanamide

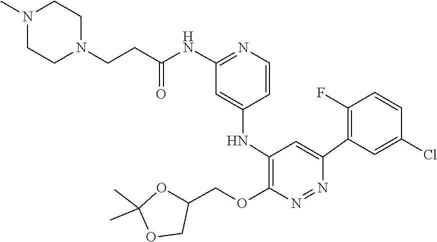

Example 77 was prepared following the procedure used for the synthesis of Example 1, starting from 6-(5-chloro-2-fluorophenyl)-3-[(2,2-dimethyl-1,3-dioxolan-4-yl)methoxy]pyridazin-4-amine (Intermediate 159, 170 mg, 0.48 mmol) and N-(4-bromopyridin-2-yl)-3-(4-methylpiperazin-1-yl)propanamide (Intermediate 2, 173 mg, 0.53 mmol) to afford title compound (260 mg, 0.43 mmol, 90% yield).

LC-MS (ESI): m/z (M+1): 600.3 (Method 2)

¹H NMR (400 MHz, Chloroform-d) δ ppm 11.22 (s, 1H), 8.24 (d, J=5.7 Hz, 1H), 8.06-8.13 (m, 2H), 7.78 (d, J=1.3 Hz, 1H), 7.37 (ddd, J=8.8, 4.2, 2.9 Hz, 1H), 7.13 (dd, J=10.4, 8.9 Hz, 1H), 7.07 (s, 1H), 6.94 (dd, J=5.6, 2.1 Hz, 1H), 4.82 (dd, J=11.1, 3.2 Hz, 1H), 4.66-4.74 (m, 1H), 4.59-4.66 (m, 1H), 4.21-4.30 (m, 1H), 3.92 (dd, J=8.6, 5.7 Hz, 1H), 2.72-2.81 (m, 2H), 2.46-2.72 (m, 10H), 2.37 (s, 3H), 1.53 (s, 3H), 1.44 (s, 3H).

### Example 78: N-(4-{[6-(5-chloro-2-fluorophenyl)-3-(2,3-dihydroxypropoxy)pyridazin-4-yl]amino}pyridin-2-yl)-3-(4-methylpiperazin-1-yl)propanamide

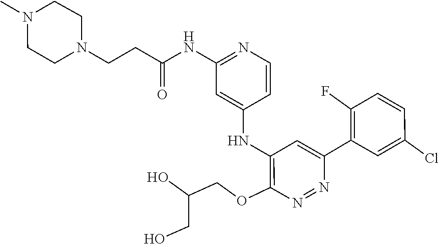

A solution of N-(4-{[6-(5-chloro-2-fluorophenyl)-3-[(2,2-dimethyl-1,3-dioxolan-4-yl)methoxy]pyridazin-4-yl]amino}pyridin-2-yl)-3-(4-methylpiperazin-1-yl)propanamide (Example 77, 21.5 mg, 0.04 mmol) in 0.5 N HCl aqueous solution (0.36 mL, 0.18 mmol) and MeOH (0.36 mL) was stirred at RT overnight. The mixture was diluted with Na₂CO₃ sat. sol. (final pH=basic), then extracted with EtOAc (3×). The combined organic layers were filtered through a phase separator and evaporated under vacuum, affording title compound (14 mg, 0.025 mmol, 70% yield).

LC-MS (ESI): m/z (M+1): 560.3 (Method 2)

¹H NMR (400 MHz, Chloroform-d) δ ppm 11.24 (s, 1H), 8.22 (d, J=5.7 Hz, 1H), 7.97-8.09 (m, 2H), 7.74 (s, 1H), 7.31-7.41 (m, 1H), 7.27-7.30 (m, 1H), 7.12 (dd, J=10.3, 9.0 Hz, 1H), 6.94 (dd, J=5.7, 2.0 Hz, 1H), 4.80-4.88 (m, 1H), 4.72-4.79 (m, 1H), 4.20-4.28 (m, 1H), 3.82-3.89 (m, 1H), 3.73-3.81 (m, 1H), 2.72-2.78 (m, 2H), 2.52-2.57 (m, 2H), 2.42-2.94 (m, 8H), 2.35 (s, 3H).

### Example 79: N-(4-{[6-(5-chloro-2-fluorophenyl)-3-[(2-oxo-1,3-dioxolan-4-yl)methoxy]pyridazin-4-yl]amino}pyridin-2-yl)-3-(4-methylpiperazin-1-yl)propanamide

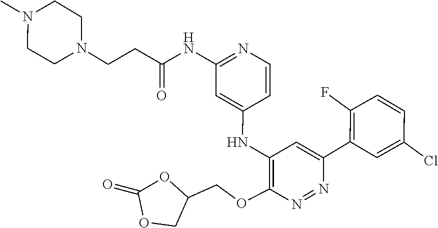

A suspension of N-(4-{[6-(5-chloro-2-fluorophenyl)-3-(2,3-dihydroxypropoxy)pyridazin-4-yl]amino}pyridin-2-yl)-3-(4-methylpiperazin-1-yl)propanamide (Example 78, 20 mg, 0.04 mmol) and 1,1′-carbonyldiimidazole (7 mg, 0.04 mmol) in methyl ethyl ketone (1.8 mL) was stirred at RT for 8 hrs. Further 1,1′-carbonyldiimidazole (7 mg, 0.04 mmol) was added, and the reaction was stirred overnight at RT. Volatiles were removed under vacuum. The crude material was purified by reverse flash chromatography on Biotage C18 cartridge (from H₂O+0.1% NH₄OH to 55% MeCN). Fraction containing the desired product were collected, concentrated under vacuum to remove the excess of MeCN then freeze-dried to afford title compound (0.5 mg, 0.01 mmol, 26% yield).

LC-MS (ESI): m/z (M+1): 586.3 (Method 2)

¹H NMR (500 MHz, Chloroform-d) δ ppm 11.24 (br s, 1H), 8.25 (d, J=5.5 Hz, 1H), 8.17 (d, J=1.9 Hz, 1H), 8.05 (dd, J=6.7, 2.7 Hz, 1H), 7.82 (d, J=1.1 Hz, 1H), 7.39 (ddd, J=8.7, 4.2, 2.9 Hz, 1H), 7.15 (dd, J=10.4, 8.9 Hz, 1H), 6.90 (dd, J=5.6, 2.1 Hz, 1H), 6.83 (s, 1H), 5.28 (dtd, J=8.2, 5.4, 5.4, 3.0 Hz, 1H), 4.95-5.01 (m, 1H), 4.87-4.95 (m, 1H), 4.73 (t, J=8.6 Hz, 1H), 4.49 (dd, J=8.9, 5.6 Hz, 1H), 2.74-2.80 (m, 2H), 2.54-2.60 (m, 2H), 2.45-2.88 (m, 8H), 2.38 (s, 3H).

### Example 80: N-(4-{[6-(5-chloro-2-fluorophenyl)-3-[3-(hydroxymethyl)cyclobutoxy]pyridazin-4-yl]amino}pyridin-2-yl)-3-(4-methylpiperazin-1-yl)propanamide

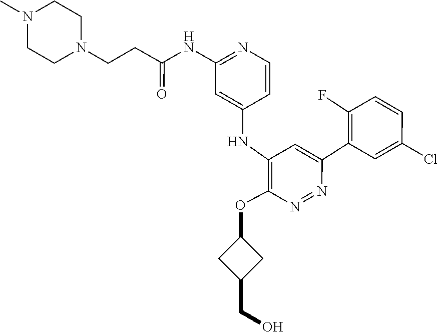

Example 80 was prepared following the procedure used for the synthesis of Example 1, starting from 3-{[4-amino-6-(5-chloro-2-fluorophenyl)pyridazin-3-yl]oxy}cyclobutyl)methanol (Intermediate 164, 40 mg, 0.12 mmol) and N-(4-bromopyridin-2-yl)-3-(4-methylpiperazin-1-yl)propanamide (Intermediate 2, 45 mg, 0.14 mmol) to afford title compound (22 mg, 0.04 mmol, 32% yield).

LC-MS (ESI): m/z (M+1): 570.4 (Method 2)

¹H NMR (500 MHz, Chloroform-d) δ ppm 11.20 (s, 1H), 8.23 (d, J=5.6 Hz, 1H), 8.10 (dd, J=6.7, 2.7 Hz, 1H), 8.05 (d, J=1.9 Hz, 1H), 7.75 (d, J=1.5 Hz, 1H), 7.35 (ddd, J=8.7, 4.2, 2.7 Hz, 1H), 7.11 (dd, J=10.6, 8.8 Hz, 1H), 6.96 (dd, J=5.6, 2.2 Hz, 1H), 6.88 (s, 1H), 5.47 (quin, J=7.4 Hz, 1H), 3.73 (d, J=5.8 Hz, 2H), 2.75 (br dd, J=6.5, 5.3 Hz, 4H), 2.53-2.57 (m, 2H), 2.61 (s, 8H), 2.36 (s, 3H), 2.24-2.34 (m, 1H), 2.04-2.15 (m, 2H).

### Example 81: N-(4-{[6-(5-chloro-2-fluorophenyl)-3-[(3-hydroxyphenyl)methoxy]pyridazin-4-yl]amino}pyridin-2-yl)-3-(4-methylpiperazin-1-yl)propanamide

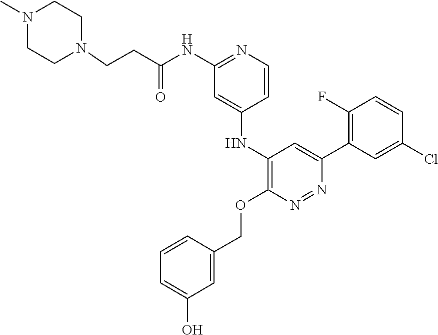 [SMILES: CN1CCN(CCC(=O)Nc2cc(Nc3cc(-c4cc(Cl)ccc4F)nnc3OCc3cccc(O)c3)ccn2)CC1]

Example 81 was prepared following the procedure used for the synthesis of Example 1, starting from 3-({[4-amino-6-(5-chloro-2-fluorophenyl)pyridazin-3-yl]oxy}methyl)phenol (Intermediate 166, 34 mg, 0.10 mmol) and N-(4-bromopyridin-2-yl)-3-(4-methylpiperazin-1-yl)propanamide (Intermediate 2, 35 mg, 0.11 mmol) to afford title compound (40 mg, 0.07 mmol, 69% yield).

LC-MS (ESI): m/z (M+1): 592.2 (Method 2)

¹H NMR (500 MHz, Chloroform-d) δ ppm 11.20 (s, 1H), 8.22 (d, J=5.6 Hz, 1H), 8.08 (dd, J=6.7, 2.7 Hz, 1H), 8.03 (d, J=1.9 Hz, 1H), 7.75 (d, J=1.4 Hz, 1H), 7.34-7.40 (m, 1H), 7.27-7.32 (m, 1H), 7.04-7.16 (m, 3H), 6.96 (s, 1H), 6.94 (dd, J=5.6, 2.2 Hz, 1H), 6.87 (ddd, J=8.1, 2.3, 1.0 Hz, 1H), 5.82-6.55 (m, 1H), 5.66 (s, 2H), 2.72-2.76 (m, 2H), 2.52-2.57 (m, 2H), 2.46-2.93 (m, 8H), 2.36 (s, 3H).

### Example 82: N-(4-{[6-(5-chloro-2-fluorophenyl)-3-[(2-hydroxyethyl)sulfanyl]pyridazin-4-yl]amino}pyridin-2-yl)-2-{6-methyl-3,6-diazabicyclo[3.2.2]nonan-3-yl}acetamide

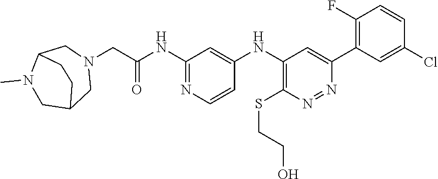 [SMILES: CN1CC2CCC1CN(CC(=O)Nc1cc(Nc3cc(-c4cc(Cl)ccc4F)nnc3SCCO)ccn1)C2]

N-(4-bromopyridin-2-yl)-2-{6-methyl-3,6-diazabicyclo[3.2.2]nonan-3-yl}acetamide (Intermediate 169, 94 mg, 0.27 mmol) was added to a stirred mixture of 3-({2-[(tert-butyldimethylsilyl)oxy]ethyl}sulfanyl)-6-(5-chloro-2-fluorophenyl)pyridazin-4-amine (Intermediate 67, 100 mg, 0.24 mmol), Pd(OAc)₂ (3.6 mg, 0.02 mmol), Xantphos (17 mg, 0.03 mmol) and Cs₂CO₃ (158 mg, 0.48 mmol) in dry 1,2-dimethoxyethane (4 mL) at RT. The mixture was degassed with N₂. The vial was closed, and the reaction was heated at 100° C. for 6 hrs. The conversion was only partial, but the reaction was stopped. The mixture was evaporated and then partitioned between DCM and brine. The organic phase was separated, dried over Na₂SO₄, and filtered. The solvent was evaporated to give an orange oil which was purified by reverse flash chromatography on Biotage C18 cartridge (from H₂O+0.1% HCOOH to 50% MeCN+0.1% HCOOH). Opportune fractions were collected and evaporated. During evaporation deprotection occurred, the material recovered was further purified by HPLC purification in acid conditions, fractions were concentrated at low volume and eluted through a PL-HCO3 cartridge using MeOH to afford, after evaporation, title compound (13 mg, 0.02 mmol, 10% yield).

LC-MS (ESI): m/z (M+1): 572.3 (Method 1)

¹H NMR (400 MHz, Chloroform-d) δ ppm 9.83 (s, 1H), 8.26 (d, J=5.7 Hz, 1H), 8.14 (dd, J=6.7, 2.7 Hz, 1H), 8.10 (d, J=2.0 Hz, 1H), 7.74 (s, 1H), 7.40 (ddd, J=8.7, 4.1, 3.0 Hz, 1H), 7.14 (dd, J=10.4, 8.9 Hz, 1H), 6.94 (dd, J=5.7, 2.0 Hz, 1H), 6.54 (s, 1H), 4.07 (t, J=5.6 Hz, 2H), 3.66 (t, J=5.6 Hz, 2H), 3.24 (s, 2H), 2.70-3.06 (m, 7H), 2.47 (s, 3H), 2.00-2.19 (m, 4H), 1.74-1.84 (m, 1H).

### Example 83: Cis N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)cyclobutane-1-carboxamide

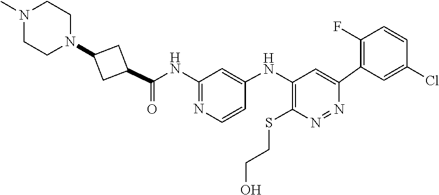

Example 83 was prepared following the procedure used for the synthesis of Example 23 starting from cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)cyclobutane-1-carboxamide (Intermediate 172, 39 mg, 0.06 mmol) to afford title compound (21 mg, 0.04 mmol, 65% yield).

LC-MS (ESI): m/z (M+1): 572.3 (Method 2)

¹H NMR (400 MHz, Chloroform-d) δ ppm 8.94 (s, 1H), 8.18-8.26 (m, 1H), 8.15 (dd, J=6.6, 2.6 Hz, 1H), 8.08 (d, J=1.5 Hz, 1H), 7.73 (s, 1H), 7.37-7.44 (m, 1H), 7.14 (dd, J=10.2, 9.1 Hz, 1H), 6.93 (dd, J=5.6, 1.9 Hz, 1H), 6.54 (s, 1H), 4.07 (t, J=5.5 Hz, 2H), 3.62-3.70 (m, 2H), 3.40 (br s, 1H), 2.92 (quin, J=8.3 Hz, 1H), 2.82 (quin, J=7.2 Hz, 1H), 2.37-2.74 (m, 10H), 2.33 (s, 3H), 2.17-2.29 (m, 2H).

### Example 84: N-(4-{[6-(5-chloro-2-fluorophenyl)-3-[(2-hydroxyethyl)sulfanyl]pyridazin-4-yl]amino}pyridin-2-yl)-3-(4-methyl-1,4-diazepan-1-yl)propanamide

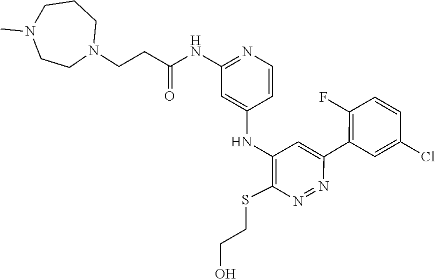

Example 84 was prepared following the procedure used for the synthesis of Example 23 starting from N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methyl-1,4-diazepan-1-yl)propanamide (Intermediate 174, 94 mg, 0.14 mmol) to afford title compound (43 mg, 0.08 mmol, 55% yield).

LC-MS (ESI): m/z (M+1): 560.2 (Method 2)

¹H NMR (400 MHz, Chloroform-d) δ 11.61 (s, 1H), 8.24 (d, J=5.5 Hz, 1H), 8.14 (dd, J=6.6, 2.6 Hz, 1H), 8.06 (d, J=1.8 Hz, 1H), 7.73 (s, 1H), 7.34-7.45 (m, 1H), 7.13 (dd, J=10.4, 8.9 Hz, 1H), 6.91 (dd, J=5.7, 2.0 Hz, 1H), 6.51 (s, 1H), 4.07 (br t, J=5.0 Hz, 2H), 3.66 (t, J=5.5 Hz, 2H), 3.23-3.47 (m, 1H), 2.83-2.93 (m, 6H), 2.74-2.83 (m, 4H), 2.52 (t, J=5.7 Hz, 2H), 2.42 (s, 3H), 1.97 (quin, J=5.9 Hz, 2H).

### Example 85: N-(4-{[6-(5-chloro-2-fluorophenyl)-3-{[(2,2-dimethyl-1,3-dioxolan-4-yl)methyl]sulfanyl}pyridazin-4-yl]amino}pyridin-2-yl)-3-(4-methylpiperazin-1-yl)propanamide

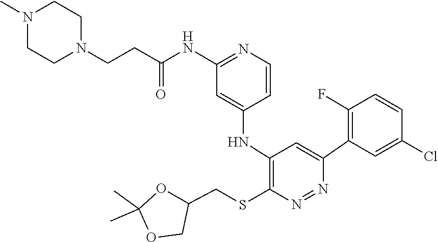

Example 85 was prepared following the procedure used for the synthesis of Example 1, starting from 6-(5-chloro-2-fluorophenyl)-3-{[(2,2-dimethyl-1,3-dioxolan-4-yl)methyl]sulfanyl}pyridazin-4-amine (Intermediate 177, 200 mg, 0.54 mmol) and N-(4-bromopyridin-2-yl)-3-(4-methylpiperazin-1-yl)propanamide (Intermediate 2, 212 mg, 0.65 mmol) to afford title compound (100 mg, 0.16 mmol, 30% yield).

LC-MS (ESI): m/z (M+1): 616.3 (Method 2)

¹H NMR (400 MHz, Chloroform-d) δ ppm 11.24 (s, 1H), 8.23 (d, J=5.7 Hz, 1H), 8.16 (dd, J=6.8, 2.6 Hz, 1H), 8.05 (d, J=1.8 Hz, 1H), 7.73 (s, 1H), 7.39 (dt, J=8.7, 3.4 Hz, 1H), 7.13 (dd, J=10.4, 9.1 Hz, 1H), 6.89 (dd, J=5.6, 1.9 Hz, 1H), 6.42 (s, 1H), 4.49-4.63 (m, 1H), 4.20 (dd, J=8.4, 6.2 Hz, 1H), 3.77-3.90 (m, 2H), 3.60 (dd, J=13.7, 7.1 Hz, 1H), 2.71-2.80 (m, 2H), 2.52-2.59 (m, 2H), 2.43-3.20 (m, 8H), 2.37 (s, 3H), 1.50 (s, 3H), 1.38 (s, 3H).

### Example 86: N-(4-{[6-(5-chloro-2-fluorophenyl)-3-[(2,3-dihydroxypropyl)sulfanyl]pyridazin-4-yl]amino}pyridin-2-yl)-3-(4-methylpiperazin-1-yl)propanamide

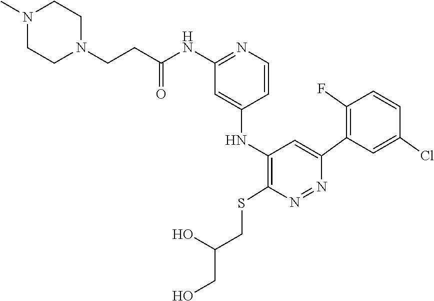

TFA (0.07 mL, 0.97 mmol) was added to a stirred solution of N-(4-{[6-(5-chloro-2-fluorophenyl)-3-{[(2,2-dimethyl-1,3-dioxolan-4-yl)methyl]sulfanyl}pyridazin-4-yl]amino}pyridin-2-yl)-3-(4-methylpiperazin-1-yl)propanamide (Example 85, 60 mg, 0.10 mmol) in DCM (2 mL) at RT. After 24 hours the solvent was removed by reduced pressure. The residue was treated with saturated NaHCO₃ aqueous solution and extracted with DCM. Organic layer was separated, dried over Na₂SO₄ and evaporated to afford title compound (47 mg, 0.08 mmol, 84% yield). LC-MS (ESI): m/z (M+1): 576.3 (Method 2)

¹H NMR (400 MHz, Chloroform-d) δ ppm 11.30 (s, 1H), 8.24 (d, J=5.7 Hz, 1H), 8.10 (dd, J=6.6, 2.4 Hz, 1H), 8.06 (s, 1H), 7.71 (s, 1H), 7.34-7.45 (m, 1H), 7.05-7.21 (m, 1H), 6.90 (dd, J=5.5, 1.5 Hz, 1H), 6.56 (s, 1H), 4.06-4.21 (m, 1H), 3.77 (qd, J=11.4, 4.5 Hz, 2H), 3.64-3.71 (m, 1H), 3.50-3.62 (m, 1H), 2.73-2.79 (m, 2H), 2.53-2.59 (m, 2H), 2.43-2.85 (m, 8H), 2.37 (s, 3H).

### Example 87: Cis N-(4-{[6-(5-chloro-2-fluorophenyl)-3-[(2-hydroxyethyl)sulfanyl]pyridazin-4-yl]amino}pyridin-2-yl)-3-[(1S,4S)-5-methyl-2,5-diazabicyclo[2.2.1]heptan-2-yl]cyclobutane-1-carboxamide

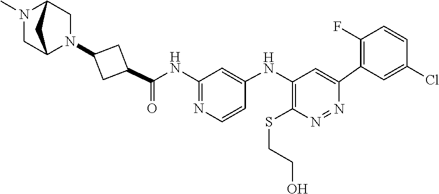

Example 87 was prepared following the procedure used for the synthesis of Example 23 starting from cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-[(1S,4S)-5-methyl-2,5-diazabicyclo[2.2.1]heptan-2-yl]cyclobutane-1-carboxamide (Intermediate 180, 56 mg, 0.08 mmol) to afford title compound (24 mg, 0.04 mmol, 50% yield).

LC-MS (ESI): m/z (M+1): 584.3 (Method 2)

¹H NMR (500 MHz, Chloroform-d) δ ppm 11.61 (br s, 1H), 8.23 (d, J=5.6 Hz, 1H), 8.14 (dd, J=6.7, 2.7 Hz, 1H), 8.08 (d, J=1.9 Hz, 1H), 7.73 (d, J=1.2 Hz, 1H), 7.39 (ddd, J=8.8, 4.3, 2.7 Hz, 1H), 7.13 (dd, J=10.6, 8.8 Hz, 1H), 6.91 (dd, J=5.6, 2.2 Hz, 1H), 6.53 (s, 1H), 4.07 (t, J=5.6 Hz, 2H), 3.65 (t, J=5.6 Hz, 2H), 3.45 (br s, 1H), 3.45 (br s, 1H), 3.32 (br s, 1H), 3.27-3.31 (m, 1H), 3.08 (dqd, J=9.2, 4.6, 4.6, 4.6, 3.6 Hz, 1H), 3.01 (br d, J=9.9 Hz, 1H), 2.95 (br d, J=10.2 Hz, 1H), 2.70 (br d, J=8.1 Hz, 1H), 2.65 (dd, J=10.0, 2.3 Hz, 1H), 2.51-2.64 (m, 2H), 2.48 (s, 3H), 2.14 (dt, J=11.9, 3.3 Hz, 2H), 1.94 (br d, J=9.7 Hz, 1H), 1.78 (br d, J=9.6 Hz, 1H).

### Example 88: Trans N-(4-{[6-(5-chloro-2-fluorophenyl)-3-[(2-hydroxyethyl)sulfanyl]pyridazin-4-yl]amino}pyridin-2-yl)-3-[(1S,4S)-5-methyl-2,5-diazabicyclo[2.2.1]heptan-2-yl]cyclobutane-1-carboxamide

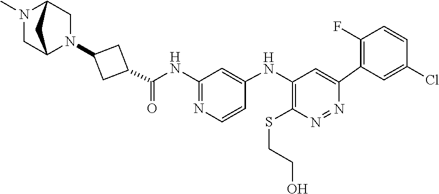

Example 88 was prepared following the procedure used for the synthesis of Example 23 starting from trans N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-[(1S,4S)-5-methyl-2,5-diazabicyclo[2.2.1]heptan-2-yl]cyclobutane-1-carboxamide (Intermediate 181, 50 mg, 0.07 mmol) to afford title compound (6.5 mg, 0.01 mmol, 16% yield). LC-MS (ESI): m/z (M+1): 584.3 (Method 2)

¹H NMR (400 MHz, Chloroform-d) δ ppm 8.20 (d, J=5.7 Hz, 1H), 8.16 (dd, J=6.7, 2.7 Hz, 1H), 8.13 (d, J=1.8 Hz, 1H), 7.84 (s, 1H), 7.75 (d, J=0.9 Hz, 1H), 7.37-7.46 (m, 1H), 7.14 (dd, J=10.4, 8.9 Hz, 1H), 6.94 (dd, J=5.6, 2.1 Hz, 1H), 6.53 (s, 1H), 4.07 (t, J=5.6 Hz, 2H), 3.66 (t, J=5.6 Hz, 2H), 3.36-3.44 (m, 1H), 3.27-3.30 (m, 1H), 3.22 (br s, 1H), 3.16-3.26 (m, 1H), 3.15-3.35 (m, 1H), 2.78 (d, J=10.3 Hz, 1H), 2.67-2.72 (m, 1H), 2.61-2.66 (m, 1H), 2.57 (dd, J=9.9, 2.4 Hz, 1H), 2.32-2.52 (m, 5H), 2.13-2.27 (m, 2H), 1.62-1.77 (m, 2H).

### Example 89: Cis N-(4-{[6-(5-chloro-2-fluorophenyl)-3-[(2-hydroxyethyl)sulfanyl]pyridazin-4-yl]amino}pyridin-2-yl)-3-(thiomorpholin-4-yl)cyclobutane-1-carboxamide

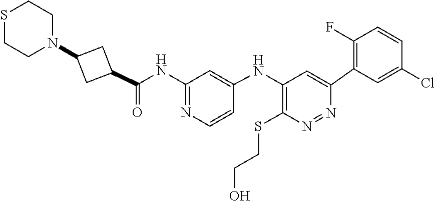

Example 89 was prepared following the procedure used for the synthesis of Example 23 starting from cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(thiomorpholin-4-yl)cyclobutane-1-carboxamide (Intermediate 184, 140 mg, 0.20 mmol) to afford title compound (37 mg, 0.07 mmol, 32% yield). LC-MS (ESI): m/z (M+1): 575.4 (Method 2)

¹H NMR (500 MHz, DMSO-d₆) δ ppm 10.35 (s, 1H), 8.89 (s, 1H), 8.10 (d, J=5.6 Hz, 1H), 8.07 (br s, 1H), 8.01 (dd, J=6.5, 2.7 Hz, 1H), 7.66 (br s, 1H), 7.57-7.63 (m, 1H), 7.42 (dd, J=10.4, 8.9 Hz, 1H), 6.88-6.96 (m, 1H), 5.09 (t, J=5.4 Hz, 1H), 3.74 (q, J=6.2 Hz, 2H), 3.50 (t, J=6.4 Hz, 2H), 2.89-3.02 (m, 1H), 2.60-2.68 (m, 1H), 2.54-2.60 (m, 4H), 2.42-2.49 (m, 4H), 2.13-2.24 (m, 2H), 1.89-2.02 (m, 2H).

### Example 90: Cis N-(4-{[6-(5-chloro-2-fluorophenyl)-3-[(2-hydroxyethyl)sulfanyl]pyridazin-4-yl]amino}pyridin-2-yl)-3-{4-methyl-4,7-diazaspiro[2.5]octan-7-yl}cyclobutane-1-carboxamide

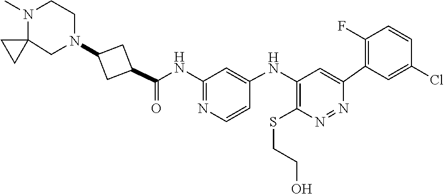

Example 90 was prepared following the procedure used for the synthesis of Example 23 starting from cis N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-{4-methyl-4,7-diazaspiro[2.5]octan-7-yl}cyclobutane-1-carboxamide (Intermediate 189, 79 mg, 0.11 mmol) to afford title compound (57 mg, 0.09 mmol, 98% yield).

LC-MS (ESI): m/z (M+1): 598.2 (Method 2)

¹H NMR (400 MHz, Chloroform-d) δ ppm 8.95 (s, 1H), 8.19 (d, J=5.6 Hz, 1H), 8.13 (dd, J=6.7, 2.6 Hz, 1H), 8.07 (d, J=1.8 Hz, 1H), 7.72 (s, 1H), 7.36-7.43 (m, 1H), 7.13 (dd, J=10.5, 8.9 Hz, 1H), 6.92 (dd, J=5.6, 2.0 Hz, 1H), 6.58 (s, 1H), 4.07 (t, J=5.5 Hz, 2H), 3.65 (t, J=5.5 Hz, 2H), 3.59 (br s, 1H), 2.96-3.04 (m, 2H), 2.87-2.97 (m, 1H), 2.81 (quin, J=7.2 Hz, 1H), 2.39-2.53 (m, 4H), 2.33 (s, 3H), 2.16-2.29 (m, 4H), 0.68-0.81 (m, 2H), 0.37-0.49 (m, 2H).

### Example 91: Cis N-(6-{[6-(5-chloro-2-fluorophenyl)-3-[(2-hydroxyethyl)sulfanyl]pyridazin-4-yl]amino}pyrimidin-4-yl)-3-(4-methylpiperazin-1-yl)cyclobutane-1-carboxamide

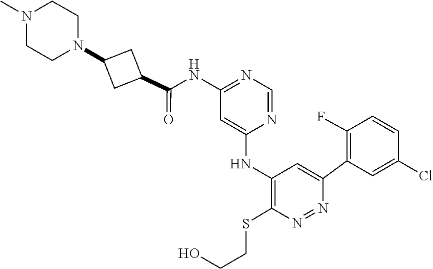

Example 91 was prepared following the procedure used for the synthesis of Example 23 starting from cis N-(6-{[3-({2-[(tert-butyldimethylsilyl)oxy]ethyl}sulfanyl)-6-(5-chloro-2-fluorophenyl)pyridazin-4-yl]amino}pyrimidin-4-yl)-3-(4-methylpiperazin-1-yl)cyclobutane-1-carboxamide (Intermediate 191, 23 mg, 0.03 mmol) to afford title compound (10 mg, 0.015 mmol, 52% yield).

LC-MS (ESI): m/z (M+1): 598.2 (Method 2)

¹H NMR (400 MHz, DMSO-d₆) δ ppm 10.70 (s, 1H), 9.48 (s, 1H), 8.44-8.50 (m, 2H), 7.99 (dd, J=6.6, 2.8 Hz, 1H), 7.97 (s, 1H), 7.60-7.68 (m, 1H), 7.47 (dd, J=10.5, 8.9 Hz, 1H), 3.70-3.76 (m, 2H), 3.47-3.53 (m, 2H), 2.63-3.75 (m, 13H), 1.96-2.41 (m, 4H).

### Example 92: methyl 5-[(4-{[6-(5-chloro-2-fluorophenyl)-3-[(2-hydroxyethyl)sulfanyl]pyridazin-4-yl]amino}pyridin-2-yl)amino]-3-(1-methylpiperidin-4-yl)thiophene-2-carboxylate

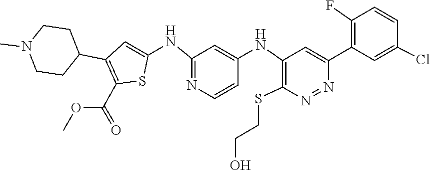 [SMILES: COC(=O)c1sc(Nc2cc(Nc3cc(-c4cc(Cl)ccc4F)nnc3SCCO)ccn2)cc1C1CCN(C)CC1]

Example 92 was prepared following the procedure used for the synthesis of Example 23 starting from methyl 5-{[(tert-butoxy)carbonyl](4-{[3-({2-[(tert-butyldimethylsilyl)oxy]ethyl}sulfanyl)-6-(5-chloro-2-fluorophenyl)pyridazin-4-yl]amino}pyridin-2-yl)amino}-3-(1-methylpiperidin-4-yl)thiophene-2-carboxylate (Intermediate 199, 40 mg, 0.05 mmol) to afford title compound (23 mg, 0.037 mmol, 74% yield). LC-MS (ESI): m/z (M+1): 629.4 (Method 2)

¹H NMR (500 MHz, DMSO-d₆) δ ppm 10.63 (br s, 1H), 8.80 (br s, 1H), 8.13 (br d, J=5.8 Hz, 1H), 8.00 (dd, J=6.5, 2.7 Hz, 1H), 7.64-7.71 (m, 1H), 7.58-7.63 (m, 1H), 7.45 (dd, J=10.5, 8.9 Hz, 1H), 6.77 (br d, J=4.4 Hz, 1H), 6.63 (s, 1H), 6.48 (s, 1H), 5.09 (br t, J=5.2 Hz, 1H), 3.72-3.77 (m, 2H), 3.71 (s, 3H), 3.50 (br t, J=6.1 Hz, 2H), 3.42 (tt, J=12.0, 3.6 Hz, 1H), 2.84 (br d, J=11.3 Hz, 2H), 2.17 (s, 3H), 1.89-1.98 (m, 2H), 1.71 (br d, J=12.5 Hz, 2H), 1.56 (qd, J=12.2, 3.6 Hz, 2H).

### Example 93: N-(4-{[6-(5-chloro-2-fluorophenyl)-3-[(1-hydroxy-2-methylpropan-2-yl)sulfanyl]pyridazin-4-yl]amino}pyridin-2-yl)-3-(4-methylpiperazin-1-yl)propanamide

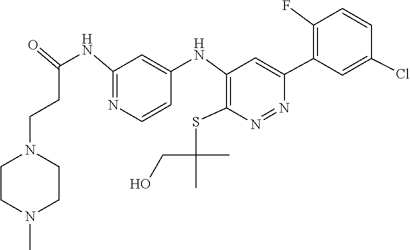

Example 93 was prepared following the procedure used for the synthesis of Example 23 starting from N-(4-{[3-({1-[(tert-butyldimethylsilyl)oxy]-2-methylpropan-2-yl}sulfanyl)-6-(5-chloro-2-fluorophenyl)pyridazin-4-yl]amino}pyridin-2-yl)-3-(4-methylpiperazin-1-yl)propanamide (Intermediate 204, 44 mg, 0.06 mmol) to afford title compound (19 mg, 0.03 mmol, 51% yield). LC-MS (ESI): m/z (M+1): 574.4 (Method 2)

¹H NMR (400 MHz, Chloroform-d) δ ppm 11.28 (s, 1H), 8.25 (d, J=5.7 Hz, 1H), 8.18 (dd, J=6.5, 1.9 Hz, 1H), 8.08 (s, 1H), 7.79 (s, 1H), 7.52 (s, 1H), 7.32-7.47 (m, 1H), 7.14 (t, J=9.6 Hz, 1H), 6.94 (br d, J=5.5 Hz, 1H), 4.58 (br t, J=5.9 Hz, 1H), 3.71 (br d, J=5.3 Hz, 2H), 2.72-2.78 (m, 2H), 2.49-2.58 (m, 2H), 2.45-2.87 (m, 8H), 2.36 (s, 3H), 1.48 (s, 6H).

### Example 94: Cis N-(4-{[6-(5-chloro-2-fluorophenyl)-3-[(1-hydroxy-2-methylpropan-2-yl)sulfanyl]pyridazin-4-yl]amino}pyridin-2-yl)-3-(4-methylpiperazin-1-yl)cyclobutane-1-carboxamide

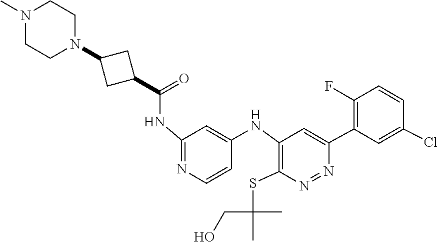

Example 94 was prepared following the procedure used for the synthesis of Example 23 starting from cis N-(4-{[3-({1-[(tert-butyldimethylsilyl)oxy]-2-methylpropan-2-yl}sulfanyl)-6-(5-chloro-2-fluorophenyl)pyridazin-4-yl]amino}pyridin-2-yl)-3-(4-methylpiperazin-1-yl)cyclobutane-1-carboxamide (Intermediate 205, 60 mg, 0.08 mmol) to afford title compound (30 mg, 0.05 mmol, 60% yield).

LC-MS (ESI): m/z (M+1): 600.3 (Method 2)

¹H NMR (500 MHz, Chloroform-d) δ ppm 8.94 (s, 1H), 8.23 (d, J=5.6 Hz, 1H), 8.19 (dd, J=6.7, 2.6 Hz, 1H), 8.11 (d, J=1.4 Hz, 1H), 7.81 (s, 1H), 7.56 (s, 1H), 7.42 (ddd, J=8.6, 4.0, 2.9 Hz, 1H), 7.15 (dd, J=10.4, 9.0 Hz, 1H), 6.98 (dd, J=5.6, 1.9 Hz, 1H), 4.56 (br t, J=5.4 Hz, 1H), 3.72 (br d, J=5.6 Hz, 2H), 2.92 (quin, J=8.3 Hz, 1H), 2.83 (quin, J=7.1 Hz, 1H), 2.42-2.50 (m, 2H), 2.33 (s, 3H), 2.30-2.75 (m, 8H), 2.20-2.29 (m, 2H), 1.49 (s, 6H).

### Example 95: N-(4-{[6-(5-chloro-2-fluorophenyl)-3-[3-(hydroxymethyl)azetidin-1-yl]pyridazin-4-yl]amino}pyridin-2-yl)-3-(4-methylpiperazin-1-yl)propanamide

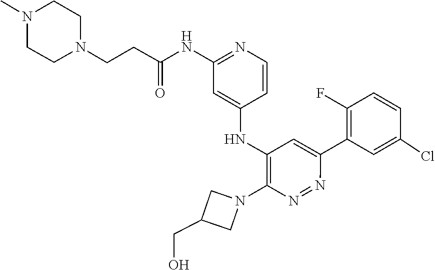 [SMILES: CN1CCN(CCC(=O)Nc2cc(Nc3cc(-c4cc(Cl)ccc4F)nnc3N3CC(CO)C3)ccn2)CC1]

Example 95 was prepared following the procedure used for the synthesis of Example 22 starting from N-(4-{[3-(3-{[(tert-butyldimethylsilyl)oxy]methyl}azetidin-1-yl)-6-(5-chloro-2-fluorophenyl)pyridazin-4-yl]amino}pyridin-2-yl)-3-(4-methylpiperazin-1-yl)propanamide (Intermediate 211, 30 mg, 0.04 mmol) to afford title compound (5 mg, 0.01 mmol, 25% yield). LC-MS (ESI): m/z (M+1): 555.4 (Method 2)

¹H NMR (400 MHz, Chloroform-d) δ ppm 11.10 (s, 1H), 8.09-8.21 (m, 2H), 7.95 (d, J=2.09 Hz, 1H), 7.68 (d, J=1.54 Hz, 1H), 7.33 (ddd, J=8.78, 4.21, 2.75 Hz, 1H), 7.09 (dd, J=10.62, 8.75 Hz, 1H), 6.78 (dd, J=5.72, 2.20 Hz, 1H), 6.36 (s, 1H), 4.34 (t, J=8.36 Hz, 2H), 4.10 (dd, J=8.53, 5.45 Hz, 2H), 3.90 (d, J=6.05 Hz, 2H), 2.88-3.02 (m, 1H), 2.49-2.83 (m, 12H), 2.37 (s, 3H).

### Example 96 (trans) and Example 97 (cis): N-(4-{[6-(5-chloro-2-fluorophenyl)-3-(dimethylamino)pyridazin-4-yl]amino}pyridin-2-yl)-3-(4-methylpiperazin-1-yl)cyclobutane-1-carboxamide (single diasteroisomers)

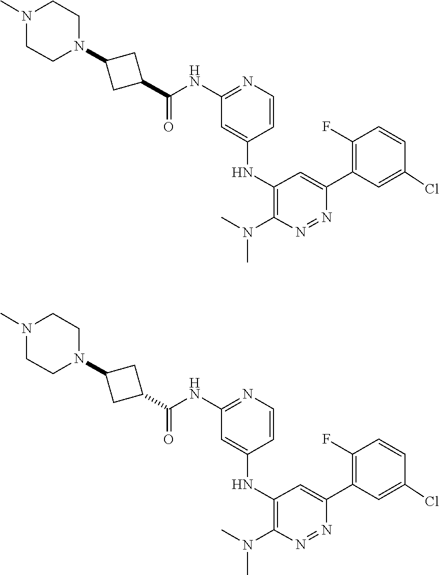 [SMILES: CN1CCN([C@H]2C[C@@H](C(=O)Nc3cc(Nc4cc(-c5cc(Cl)ccc5F)nnc4N(C)C)ccn3)C2)CC1 ; CN1CCN([C@H]2C[C@H](C(=O)Nc3cc(Nc4cc(-c5cc(Cl)ccc5F)nnc4N(C)C)ccn3)C2)CC1]

Diasteroisomeric mixture of cis/trans N-(4-{[6-(5-chloro-2-fluorophenyl)-3-(dimethylamino)pyridazin-4-yl]amino}pyridin-2-yl)-3-(4-methylpiperazin-1-yl)cyclobutane-1-carboxamide (24 mg, 0.04 mmol, 34% yield) was prepared following the procedure used for the synthesis of Example 1 starting from 6-(5-chloro-2-fluorophenyl)-N3,N3-dimethylpyridazine-3,4-diamine (Intermediate 97, 34 mg, 0.13 mmol) and N-(4-bromopyridin-2-yl)-3-(4-methylpiperazin-1-yl)cyclobutane-1-carboxamide (Intermediate 171, 54 mg, 0.15 mmol).

The mixture was separated into the single diasteroisomers by preparative chiral HPLC.

### Conditions:

| Column | Chiralpak AS-H (25 × 2.0 cm), 5 μ | |
|---|---|---|
| Mobile phase | n-Hexane/(Ethanol + 0.1% | |
| | isopropylamine) 70/30% v/v | |
| Flow rate (mL/min) | 17 | mL/min |
| DAD detection | 220 | nm |
| Loop | 1000 | μL |

Example 96 trans N-(4-{[6-(5-chloro-2-fluorophenyl)-3-(dimethylamino)pyridazin-4-yl]amino}pyridin-2-yl)-3-(4-methylpiperazin-1-yl)cyclobutane-1-carboxamide was obtained as first eluted diasteroisomer (3 mg).

Rt.=4 min, de 100%; LC-MS (ESI): m/z (M+1): 539.4 (Method 2)

¹H NMR (400 MHz, Chloroform-d) δ ppm 8.17-8.23 (m, 2H), 8.11 (d, J=1.8 Hz, 1H), 7.89 (s, 1H), 7.81 (d, J=1.1 Hz, 1H), 7.37 (ddd, J=8.7, 4.0, 2.9 Hz, 1H), 7.12 (dd, J=10.7, 8.8 Hz, 1H), 6.97 (dd, J=5.7, 2.0 Hz, 1H), 6.90 (s, 1H), 3.02-3.11 (m, 2H), 2.96 (s, 6H), 2.42-2.52 (m, 2H), 2.32 (s, 3H), 2.22-2.38 (m, 2H), 2.08-2.78 (m, 8H).

Example 97 cis N-(4-{[6-(5-chloro-2-fluorophenyl)-3-(dimethylamino)pyridazin-4-yl]amino}pyridin-2-yl)-3-(4-methylpiperazin-1-yl)cyclobutane-1-carboxamide was obtained as first eluted diasteroisomer (14.5 mg)

Rt.=5.9 min, de 99%; LC-MS (ESI): m/z (M+1): 539.4 (Method 2)

¹H NMR (400 MHz, Chloroform-d) δ ppm 8.79 (s, 1H), 8.13-8.30 (m, 2H), 7.98-8.07 (m, 1H), 7.80 (d, J=1.3 Hz, 1H), 7.37 (ddd, J=8.8, 4.2, 2.9 Hz, 1H), 7.13 (dd, J=10.6, 8.9 Hz, 1H), 6.96 (dd, J=5.7, 2.2 Hz, 1H), 6.88 (s, 1H), 2.96 (s, 6H), 2.91 (t, J=8.4 Hz, 1H), 2.82 (t, J=7.3 Hz, 1H), 2.39-2.50 (m, 2H), 2.33 (s, 3H), 2.20-2.29 (m, 2H), 2.06-2.70 (m, 8H).

### Example 98: methyl 1-[6-(5-chloro-2-fluorophenyl)-4-({2-[3-(4-methylpiperazin-1-yl)propanamido]pyridin-4-yl}amino)pyridazin-3-yl]azetidine-3-carboxylate

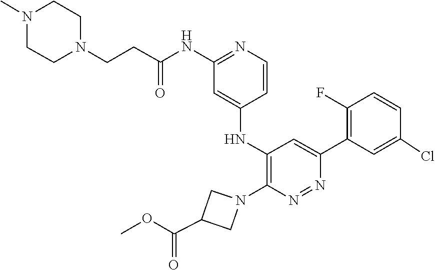 [SMILES: COC(=O)C1CN(c2nnc(-c3cc(Cl)ccc3F)cc2Nc2ccnc(NC(=O)CCN3CCN(C)CC3)c2)C1]

Example 98 was prepared following the procedure used for the synthesis of Example 2 starting from methyl 1-[4-amino-6-(5-chloro-2-fluorophenyl)pyridazin-3-yl]azetidine-3-carboxylate (Intermediate 216, 120 mg, 0.36 mmol) and N-(4-bromopyridin-2-yl)-3-(4-methylpiperazin-1-yl)propanamide (Intermediate 2, 153 mg, 0.46 mmol) at 120° C. to afford title compound (20 mg, 0.033 mmol, 10% yield).

LC-MS (ESI): m/z (M+1): 583.2 (Method 2) H NMR (400 MHz, Chloroform-d) δ ppm 11.16 (s, 1H), 8.10-8.23 (m, 2H), 7.96 (d, J=1.3 Hz, 1H), 7.72 (s, 1H), 7.30-7.39 (m, 1H), 7.03-7.16 (m, 1H), 6.77 (dd, J=5.5, 1.5 Hz, 1H), 6.06 (s, 1H), 4.44 (d, J=7.5 Hz, 4H), 3.79 (s, 3H), 3.59 (quin, J=7.6 Hz, 1H), 2.72-2.79 (m, 2H), 2.52-2.59 (m, 2H), 2.39-3.21 (m, 8H), 2.37 (s, 3H).

### Example 99: 1-[6-(5-chloro-2-fluorophenyl)-4-({2-[3-(4-methylpiperazin-1-yl)propanamido]pyridin-4-yl}amino)pyridazin-3-yl]azetidine-3-carboxylic acid

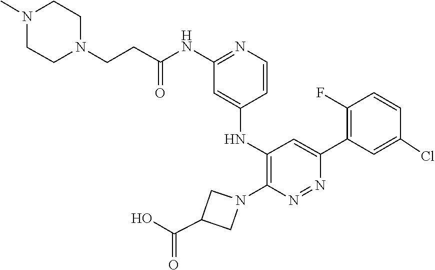 [SMILES: CN1CCN(CCC(=O)Nc2cc(Nc3cc(-c4cc(Cl)ccc4F)nnc3N3CC(C(=O)O)C3)ccn2)CC1]

A mixture of methyl 1-[6-(5-chloro-2-fluorophenyl)-4-({2-[3-(4-methylpiperazin-1-yl)propanamido]pyridin-4-yl}amino)pyridazin-3-yl]azetidine-3-carboxylate (Example 98, 20.5 mg, 0.04 mmol) and lithium hydroxide hydrate (1.62 mg, 0.04 mmol) in THE (1 mL) and H₂O (0.30 mL) was stirred at RT for 4 hrs. The mixture was evaporated, the crude material as lithium salt was purified by preparative HPLC to afford title compound (5 mg, 0.01 mmol, 25% yield). LC-MS (ESI): m/z (M+1): 569.2 (Method 2)

¹H NMR (400 MHz, DMSO-d₆) δ ppm 12.84 (br s, 1H), 10.51 (s, 1H), 8.62 (s, 1H), 8.02 (d, J=5.8 Hz, 1H), 7.94 (dd, J=6.6, 2.7 Hz, 1H), 7.88 (s, 1H), 7.60 (s, 1H), 7.54 (dt, J=7.5, 4.2 Hz, 1H), 7.31-7.43 (m, 1H), 6.69 (dd, J=5.6, 2.0 Hz, 1H), 4.16-4.36 (m, 4H), 3.39-3.50 (m, 1H), 2.56-2.63 (m, 2H), 2.48-2.55 (m, 2H), 2.20-2.48 (m, 8H), 2.14 (s, 3H).

### Example 100: propan-2-yl 1-[6-(5-chloro-2-fluorophenyl)-4-({2-[3-(4-methylpiperazin-1-yl)propanamido]pyridin-4-yl}amino)pyridazin-3-yl]azetidine-3-carboxylate

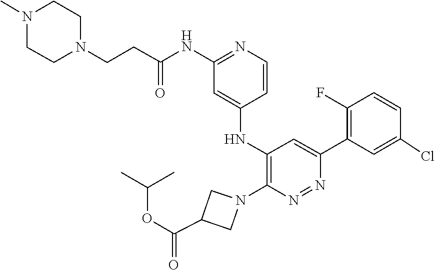 [SMILES: CC(C)OC(=O)C1CN(c2nnc(-c3cc(Cl)ccc3F)cc2Nc2ccnc(NC(=O)CCN3CCN(C)CC3)c2)C1]

### Step 1

A mixture of methyl 1-[6-(5-chloro-2-fluorophenyl)-4-({2-[3-(4-methylpiperazin-1-yl)propanamido]pyridin-4-yl}amino)pyridazin-3-yl]azetidine-3-carboxylate (Example 98, 320 mg, 0.55 mmol) and lithium hydroxide hydrate (25 mg, 0.60 mmol) in THE (7 mL) and H₂O (2.5 mL) was stirred at RT for 4 hrs. The mixture was evaporated to afford lithium 1-[6-(5-chloro-2-fluorophenyl)-4-({2-[3-(4-methylpiperazin-1-yl)propanamido]pyridin-4-yl}amino)pyridazin-3-yl]azetidine-3-carboxylic acid (0.60 mmol, quantitative yield) used as such in the next step.

### Step 2

A solution of lithium 1-[6-(5-chloro-2-fluorophenyl)-4-({2-[3-(4-methylpiperazin-1-yl)propanamido]pyridin-4-yl}amino)pyridazin-3-yl]azetidine-3-carboxylic acid (30 mg, 0.05 mmol) and HATU (28 mg, 0.07 mmol) in THE (2 mL) was treated with DIPEA (0.03 mL, 0.16 mmol) and stirred for 5 minutes. Afterwards, propan-2-ol (20 μL, 0.26 mmol) was added and the mixture stirred at 40° C. for 3 hrs. Solvent was removed under vacuum, and the crude material was purified by flash chromatography on Biotage silica NH cartridge (from c-Hex to 100% EtOAc) to afford title compound (7 mg, 0.01 mmol, 22% yield). LC-MS (ESI): m/z (M+1): 611.3 (Method 2)

¹H NMR (400 MHz, Acetone-d₆) δ ppm 10.66 (br. s., 1H), 8.07-8.13 (m, 2H), 8.03 (d, J=1.65 Hz, 1H), 7.80 (s, 1H), 7.77 (d, J=1.54 Hz, 1H), 7.47-7.53 (m, 1H), 7.31 (dd, J=10.73, 8.86 Hz, 1H), 6.82-6.87 (m, 1H), 4.98-5.10 (m, 1H), 4.44-4.52 (m, 2H), 4.36-4.43 (m, 2H), 3.55-3.64 (m, 1H), 2.72 (d, J=6.38 Hz, 2H), 2.38-2.68 (m, 10H), 2.24 (s, 3H), 1.25 (d, J=6.16 Hz, 6H).

### Example 101: N-(4-{[6-(5-chloro-2-fluorophenyl)-3-{[(3-hydroxyphenyl)methyl]amino}pyridazin-4-yl]amino}pyridin-2-yl)-3-(4-methylpiperazin-1-yl)propanamide

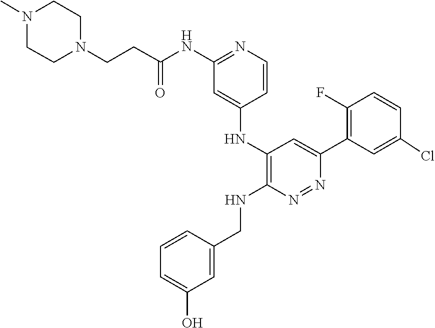 [SMILES: CN1CCN(CCC(=O)Nc2cc(Nc3cc(-c4cc(Cl)ccc4F)nnc3NCc3cccc(O)c3)ccn2)CC1]

A solution 1 M of boron tribromide in DCM (0.41 mL, 0.41 mmol) was added drop-wise to a stirred solution of N-(4-{[6-(5-chloro-2-fluorophenyl)-3-{[(3-methoxyphenyl)methyl]amino}pyridazin-4-yl]amino}pyridin-2-yl)-3-(4-methylpiperazin-1-yl)propanamide (Intermediate 222, 83 mg, 0.14 mmol) in DCM (6 mL) at RT and under N₂, then the resulting suspension was stirred at RT. After 2 hrs further 1 M of boron tribromide in DCM (0.2 mL, 0.2 mmol) was added and the reaction was stirred at RT for 3 hrs. The reaction was quenched by adding a saturated NaHCO₃ aqueous solution until pH ˜8, the mixture was separated, and the organic phase was concentrated under reduced pressure. The crude material was purified by reverse flash chromatography on Biotage C18 cartridge (from H₂O+0.1% NH₄OH to 30% MeCN), then by flash chromatography on Biotage silica NH cartridge (from DCM to 3% MeOH) to afford title compound (19 mg, 0.03 mmol, 23% yield).

LC-MS (ESI): m/z (M+1): 591.3 (Method 2)

¹H NMR (400 MHz, Chloroform-d) δ ppm 11.62 (br s, 1H), 9.26 (br s, 1H), 8.13 (d, J=5.8 Hz, 1H), 8.09 (dd, J=6.6, 2.0 Hz, 1H), 7.66 (br s, 1H), 7.56 (s, 1H), 7.50 (s, 1H), 7.23 (dt, J=8.3, 3.6 Hz, 1H), 7.14 (t, J=7.8 Hz, 1H), 6.82-6.90 (m, 2H), 6.80 (d, J=7.6 Hz, 1H), 6.69 (br d, J=8.1 Hz, 1H), 6.59 (s, 1H), 5.46 (br t, J=5.4 Hz, 1H), 4.79 (br d, J=5.2 Hz, 2H), 2.48-2.53 (m, 2H), 2.54 (br s, 8H), 2.37-2.43 (m, 2H), 2.33 (s, 3H).

### Example 102: N-(4-{[6-(5-chloro-2-fluorophenyl)-3-{[(3-hydroxyphenyl)methyl](methyl)amino}pyridazin-4-yl]amino}pyridin-2-yl)-3-(4-methylpiperazin-1-yl)propanamide

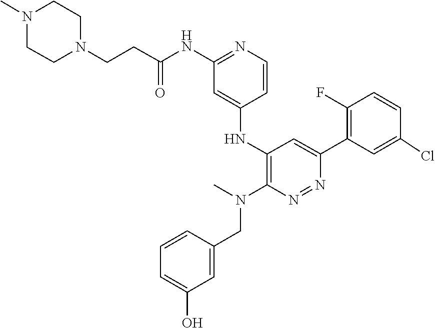 [SMILES: CN1CCN(CCC(=O)Nc2cc(Nc3cc(-c4cc(Cl)ccc4F)nnc3N(C)Cc3cccc(O)c3)ccn2)CC1]

Example 102 was prepared following the procedure used for the synthesis of Example 101 starting from N-(4-{[6-(5-chloro-2-fluorophenyl)-3-{[(3-methoxyphenyl)methyl](methyl)amino}pyridazin-4-yl]amino}pyridin-2-yl)-3-(4-methylpiperazin-1-yl)propanamide (Intermediate 225, 130 mg, 0.21 mmol) to afford title compound (5 mg, 0.01 mmol, 4% yield). LC-MS (ESI): m/z (M+1): 605.3 (Method 2)

¹H NMR (400 MHz, Chloroform-d) δ ppm 9.76 (br s, 1H), 8.15-8.25 (m, 2H), 7.94 (s, 1H), 7.80 (s, 1H), 7.35-7.44 (m, 1H), 7.33 (s, 1H), 7.20-7.26 (m, 1H), 7.09-7.19 (m, 1H), 6.99 (s, 1H), 6.95 (dd, J=5.6, 1.6 Hz, 1H), 6.91 (d, J=7.5 Hz, 1H), 6.85 (dd, J=8.2, 1.4 Hz, 1H), 4.17-4.30 (m, 2H), 4.16 (s, 2H), 3.52 (br d, J=13.4 Hz, 2H), 3.17 (br d, J=3.6 Hz, 3H), 3.10 (br d, J=13.6 Hz, 2H), 2.97 (s, 3H), 2.93 (t, J=6.1 Hz, 2H), 2.71 (br t, J=12.3 Hz, 2H), 2.64 (t, J=6.1 Hz, 2H).

### Example 103: N-(4-{[6-(5-chloro-2-fluorophenyl)-3-(dimethylamino)pyridazin-4-yl]amino}pyridin-2-yl)-2-(4-methyl-1,4-diazepan-1-yl)acetamide

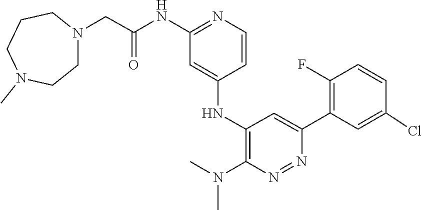 [SMILES: CN1CCCN(CC(=O)Nc2cc(Nc3cc(-c4cc(Cl)ccc4F)nnc3N(C)C)ccn2)CC1]

Example 103 was prepared following the procedure used for the synthesis of Example 1, starting from 6-(5-chloro-2-fluorophenyl)-N3,N3-dimethylpyridazine-3,4-diamine (Intermediate 97, 80 mg, 0.30 mmol) and N-(4-bromopyridin-2-yl)-2-(4-methyl-1,4-diazepan-1-yl)acetamide (Intermediate 82, 118 mg, 0.36 mmol) to afford title compound (58 mg, 0.11 mmol, 38% yield). LC-MS (ESI): m/z (M+1): 513.4 (Method 2)

¹H NMR (400 MHz, Chloroform-d) δ ppm 9.77 (s, 1H), 8.26 (d, J=5.72 Hz, 1H), 8.20 (dd, J=6.71, 2.75 Hz, 1H), 8.11 (d, J=1.98 Hz, 1H), 7.83 (d, J=1.54 Hz, 1H), 7.38 (ddd, J=8.75, 4.24, 2.86 Hz, 1H), 7.14 (dd, J=10.56, 8.80 Hz, 1H), 6.96-7.03 (m, 1H), 6.91 (s, 1H), 3.34 (s, 2H), 2.95-3.01 (m, 6H), 2.87-2.94 (m, 4H), 2.66-2.78 (m, 4H), 2.43 (s, 3H), 1.92 (quin, J=5.94 Hz, 2H).

### Example 104: N-(4-{[6-(5-chloro-2-fluorophenyl)-3-{7-oxo-6-oxa-2-azaspiro[3.4]octan-2-yl}pyridazin-4-yl]amino}pyridin-2-yl)-3-(4-methylpiperazin-1-yl)propanamide

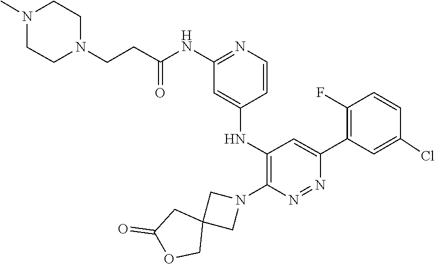 [SMILES: CN1CCN(CCC(=O)Nc2cc(Nc3cc(-c4cc(Cl)ccc4F)nnc3N3CC4(COC(=O)C4)C3)ccn2)CC1]

Example 104 was prepared following the procedure used for the synthesis of Example 1, starting from 2-[4-amino-6-(5-chloro-2-fluorophenyl)pyridazin-3-yl]-6-oxa-2-azaspiro[3.4]octan-7-one (Intermediate 231, 45 mg, 0.13 mmol) and N-(4-bromopyridin-2-yl)-3-(4-methylpiperazin-1-yl)propanamide (Intermediate 2, 44 mg, 0.13 mmol) to afford title compound (29 mg, 0.05 mmol, 38% yield).

LC-MS (ESI): m/z (M+1): 595.3 (Method 2)

¹H NMR (500 MHz, Chloroform-d) δ ppm11.24 (s, 1H), 8.20 (d, J=5.6 Hz, 1H), 8.13 (dd, J=6.7, 2.7 Hz, 1H), 7.95 (d, J=2.1 Hz, 1H), 7.73 (d, J=1.4 Hz, 1H), 7.36 (ddd, J=8.8, 4.2, 2.8 Hz, 1H), 7.11 (dd, J=10.6, 8.8 Hz, 1H), 6.76 (dd, J=5.6, 2.2 Hz, 1H), 6.02 (s, 1H), 4.55 (s, 2H), 4.21-4.40 (m, 4H), 2.89 (s, 2H), 2.74-2.80 (m, 2H), 2.53-2.58 (m, 2H), 2.46-2.81 (m, 8H), 2.37 (s, 3H).

### Example 105: N-(4-{[6-(5-chloro-2-fluorophenyl)-3-[methyl(oxolan-3-yl)amino]pyridazin-4-yl]amino}pyridin-2-yl)-3-(4-methylpiperazin-1-yl)propanamide

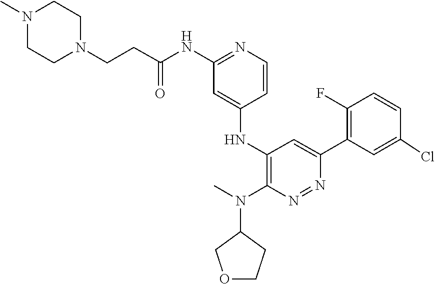 [SMILES: CN1CCN(CCC(=O)Nc2cc(Nc3cc(-c4cc(Cl)ccc4F)nnc3N(C)C3CCOC3)ccn2)CC1]

Example 105 was prepared following the procedure used for the synthesis of Example 1, starting from 6-(5-chloro-2-fluorophenyl)-N3-methyl-N3-(oxolan-3-yl)pyridazine-3,4-diamine (Intermediate 236, 76 mg, 0.22 mmol) and N-(4-bromopyridin-2-yl)-3-(4-methylpiperazin-1-yl)propanamide (Intermediate 2, 86 mg, 0.26 mmol) to afford title compound (30 mg, 0.05 mmol, 24% yield).

LC-MS (ESI): m/z (M+1): 569.3 (Method 2)

¹H NMR (500 MHz, Chloroform-d) δ ppm 11.15 (br s, 1H), 8.24 (d, J=5.7 Hz, 1H), 8.19 (dd, J=6.7, 2.7 Hz, 1H), 8.07 (d, J=1.8 Hz, 1H), 7.83 (d, J=0.9 Hz, 1H), 7.34-7.43 (m, 1H), 7.09-7.18 (m, 2H), 6.94 (dd, J=5.5, 2.0 Hz, 1H), 4.38 (quin, J=6.2 Hz, 1H), 3.92-4.11 (m, 2H), 3.75-3.90 (m, 2H), 2.83 (s, 3H), 2.74-2.80 (m, 2H), 2.54-2.61 (m, 2H), 2.49-2.87 (m, 8H), 2.39 (s, 3H), 2.25-2.35 (m, 1H), 1.95-2.07 (m, 1H).

### Example 106 (Enantiomer 1) and example 107 (Enantiomer 2): N-(4-{[6-(5-chloro-2-fluorophenyl)-3-[methyl(oxolan-3-yl)amino]pyridazin-4-yl]amino}pyridin-2-yl)-3-(4-methylpiperazin-1-yl)propanamide (single enantiomers)

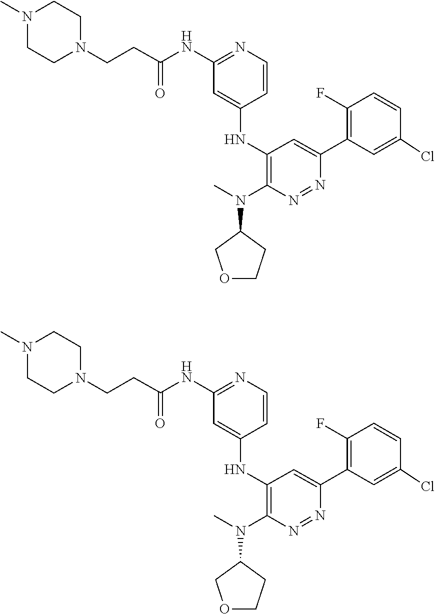 [SMILES: CN1CCN(CCC(=O)Nc2cc(Nc3cc(-c4cc(Cl)ccc4F)nnc3N(C)[C@H]3CCOC3)ccn2)CC1 ; CN1CCN(CCC(=O)Nc2cc(Nc3cc(-c4cc(Cl)ccc4F)nnc3N(C)[C@@H]3CCOC3)ccn2)CC1]

Racemate N-(4-{[6-(5-chloro-2-fluorophenyl)-3-[methyl(oxolan-3-yl)amino]pyridazin-4-yl]amino}pyridin-2-yl)-3-(4-methylpiperazin-1-yl)propanamide (Example 105, 23 mg) was separated into the single enantiomers by preparative chiral HPLC.

### Conditions:

| Column | Chiralpak AD-H (25 × 2.0 cm), 5 μ | |
|---|---|---|
| Mobile phase | n-Hexane/(Ethanol/Methanol 1/1 + 0.1% | |
| | isopropylamine), 10/90% v/v | |
| Flow rate (mL/min) | 16 | mL/min |
| DAD detection | 240 | nm |
| Loop | 1000 | μL |

Example 106 was obtained as first eluted enantiomer (9.4 mg)

Rt.=14.1 min, ee 100%; LC-MS (ESI): m/z (M+1): 569.2 (Method 2)

Example 107 was obtained as the second eluted enantiomer (9.2 mg)

Rt.=21.6 min, ee 100%; LC-MS (ESI): m/z (M+1): 569.2 (Method 2)

### Example 108: N-(4-{[6-(5-chloro-2-fluorophenyl)-3-{methyl[(2-oxooxolan-3-yl)methyl]amino}pyridazin-4-yl]amino}pyridin-2-yl)-3-(4-methylpiperazin-1-yl)propanamide

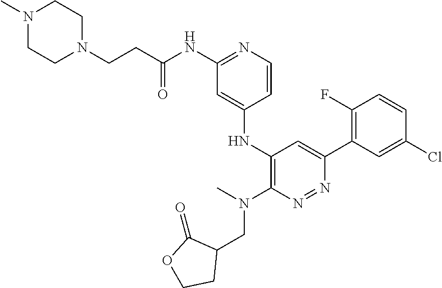 [SMILES: CN1CCN(CCC(=O)Nc2cc(Nc3cc(-c4cc(Cl)ccc4F)nnc3N(C)CC3CCOC3=O)ccn2)CC1]

Example 108 was prepared following the procedure used for the synthesis of Example 1, starting from 3-({[4-amino-6-(5-chloro-2-fluorophenyl)pyridazin-3-yl](methyl)amino}methyl)oxolan-2-one (Intermediate 241, 60 mg, 0.17 mmol) and N-(4-bromopyridin-2-yl)-3-(4-methylpiperazin-1-yl)propanamide (Intermediate 2, 67 mg, 0.21 mmol) to afford title compound (14 mg, 0.02 mmol, 14% yield).

LC-MS (ESI): m/z (M+1): 569.3 (Method 2)

¹H NMR (400 MHz, Chloroform-d) δ ppm 11.07 (s, 1H), 8.21 (d, J=5.7 Hz, 1H), 8.19 (d, J=1.3 Hz, 1H), 8.16 (dd, J=6.7, 2.7 Hz, 1H), 7.87 (s, 2H), 7.32-7.41 (m, 1H), 7.07-7.19 (m, 1H), 6.91 (dd, J=5.6, 1.9 Hz, 1H), 4.38 (td, J=8.7, 3.1 Hz, 1H), 4.25 (td, J=8.9, 7.2 Hz, 1H), 3.47-3.59 (m, 2H), 2.91-3.05 (m, 4H), 2.73-2.80 (m, 2H), 2.52-2.60 (m, 2H), 2.51-2.81 (m, 8H), 2.35-2.47 (m, 4H), 1.90-2.08 (m, 1H).

### Example 109: methyl 1-[6-(5-chloro-2-fluorophenyl)-4-[(2-{2-[(1S,4S)-5-methyl-2,5-diazabicyclo[2.2.1]heptan-2-yl]acetamido}pyridin-4-yl)amino]pyridazin-3-yl]azetidine-3-carboxylate

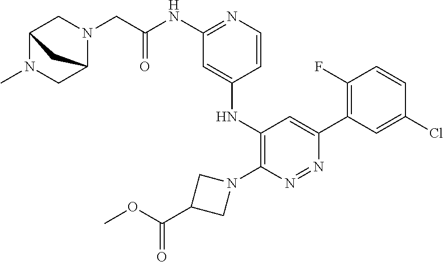 [SMILES: COC(=O)C1CN(c2nnc(-c3cc(Cl)ccc3F)cc2Nc2ccnc(NC(=O)CN3C[C@@H]4C[C@H]3CN4C)c2)C1]

Example 109 was prepared following the procedure used for the synthesis of Example 1, starting from methyl 1-[4-amino-6-(5-chloro-2-fluorophenyl)pyridazin-3-yl]azetidine-3-carboxylate (Intermediate 216, 80 mg, 0.23 mmol) and N-(4-bromopyridin-2-yl)-2-[(1S,4S)-5-methyl-2,5-diazabicyclo[2.2.1]heptan-2-yl]acetamide (Intermediate 143, 89 mg, 0.27 mmol) at 120° C. to afford title compound (48 mg, 0.08 mmol, 36% yield). LC-MS (ESI): m/z (M+1): 581.2 (Method 2)

¹H NMR (500 MHz, DMSO-d₆) δ ppm 9.67 (s, 1H), 8.73 (s, 1H), 8.03 (d, J=5.6 Hz, 1H), 7.93 (dd, J=6.6, 2.7 Hz, 1H), 7.87 (d, J=1.6 Hz, 1H), 7.63 (s, 1H), 7.50-7.58 (m, 1H), 7.40 (dd, J=10.5, 8.9 Hz, 1H), 6.73 (dd, J=5.7, 2.1 Hz, 1H), 4.32-4.41 (m, 2H), 4.26 (t, J=7.2 Hz, 2H), 3.66 (s, 3H), 3.62 (tt, J=8.8, 6.3 Hz, 1H), 3.32 (br s, 1H), 3.28 (s, 2H), 3.17 (s, 1H), 2.76 (d, J=9.5 Hz, 1H), 2.65-2.70 (m, 1H), 2.57 (s, 2H), 2.26 (s, 3H), 1.57-1.70 (m, 2H).

### Example 110: N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)propanamide

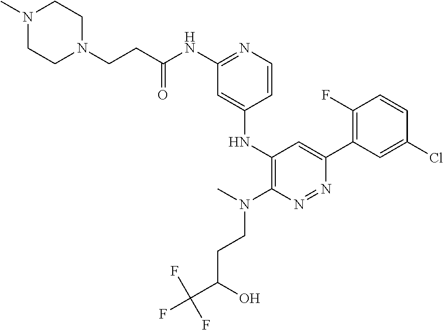 [SMILES: CN1CCN(CCC(=O)Nc2cc(Nc3cc(-c4cc(Cl)ccc4F)nnc3N(C)CCC(O)C(F)(F)F)ccn2)CC1]

Example 110 was prepared following the procedure used for the synthesis of Example 2 starting from 4-{[4-amino-6-(5-chloro-2-fluorophenyl)pyridazin-3-yl](methyl)amino}-1,1,1-trifluorobutan-2-ol (Intermediate 247, 50 mg, 0.12 mmol) and N-(4-bromopyridin-2-yl)-3-(4-methylpiperazin-1-yl)propanamide (Intermediate 2, 42 mg, 0.13 mmol) to afford title compound (38 mg, 0.06 mmol, 53% yield).

LC-MS (ESI): m/z (M+1): 625.4 (Method 2)

¹H NMR (400 MHz, DMSO-d₆) δ ppm 10.56 (s, 1H), 8.73 (s, 1H), 8.09 (d, J=5.7 Hz, 1H), 7.93-8.04 (m, 2H), 7.66 (s, 1H), 7.52-7.63 (m, 1H), 7.40 (dd, J=10.6, 8.9 Hz, 1H), 6.84 (dd, J=5.7, 2.0 Hz, 1H), 6.18 (d, J=6.8 Hz, 1H), 3.97-4.17 (m, 1H), 3.41-3.65 (m, 2H), 2.90 (s, 3H), 2.57-2.63 (m, 2H), 2.47-2.55 (m, 2H), 2.26-2.57 (m, 8H), 2.16 (br s, 3H), 1.84-2.01 (m, 1H), 1.64-1.79 (m, 1H).

### Example 111 (cis Enantiomer 1), example 112 (trans Enantiomer 1), Example 113 (cis Enantiomer 2), and Example 114 (trans Enantiomer 2): N-(4-{[6-(5-chloro-2-fluorophenyl)-3-(oxolan-3-yloxy)pyridazin-4-yl]amino}pyridin-2-yl)-3-(4-methylpiperazin-1-yl)cyclobutane-1-carboxamide single isomers

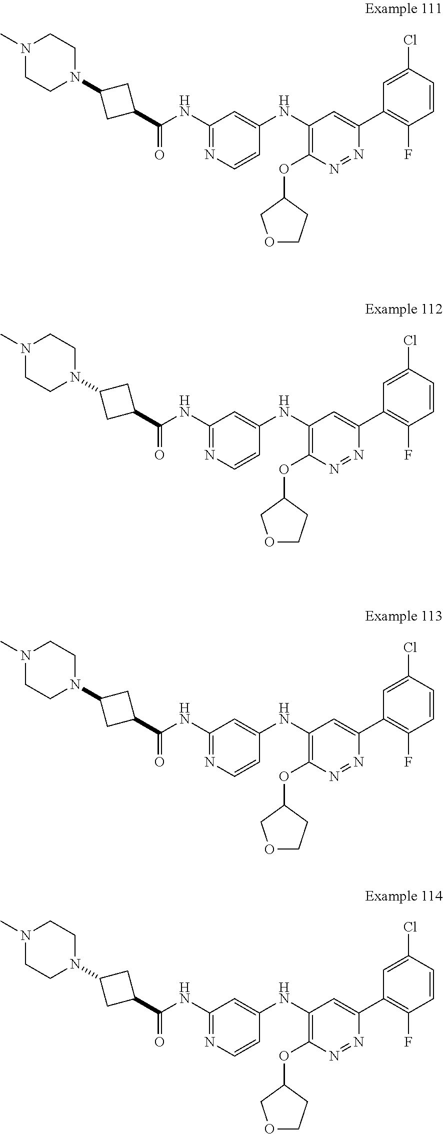

Diasteroisomeric mixture cis/trans N-(4-{[6-(5-chloro-2-fluorophenyl)-3-(oxolan-3-yloxy)pyridazin-4-yl]amino}pyridin-2-yl)-3-(4-methylpiperazin-1-yl)cyclobutane-1-carboxamide (120 mg, 0.21 mmol, 76% yield) was prepared following the procedure used for the synthesis of Example 1, starting from methyl 6-(5-chloro-2-fluorophenyl)-3-(oxolan-3-yloxy)pyridazin-4-amine (Intermediate 136, 85 mg, 0.27 mmol) and N-(4-bromopyridin-2-yl)-3-(4-methylpiperazin-1-yl)cyclobutane-1-carboxamide (Intermediate 171, 117 mg, 0.33 mmol).

The mixture was first separated into the diasteroisomeric couples (Enantiomer 1 cis/trans mixture and Enantiomer 2 cis/trans) by preparative chiral HPLC.

Conditions for first run of separation:

| Column | Chiralcel OJ-H (25 × 3.0 cm), 5 μ | |
|---|---|---|
| Mobile phase | n-Hexane/(Ethanol/Methanol + 0.1% | |
| | isopropylamine 60/40% v/v | |
| Flow rate (ml/min) | 40 | ml/min |
| DAD detection | 220 | nm |
| Loop | 500 | μL |

First eluted couple Enantiomer 1 Cis/Trans mixture (50 mg) was further separated by chiral HPLC

### Conditions:

| Column | Chiralpak AS-H (25 × 2.0 cm), 5 μ | |
|---|---|---|
| Mobile phase | n-Hexane/(Ethanol + 0.1% | |
| | isopropylamine) 70/30% v/v | |
| Flow rate (ml/min) | 17 | ml/min |
| DAD detection | 220 | nm |
| Loop | 750 | μL |

Example 111 (Cis Enantiomer 1) was obtained as the second eluted diasteroisomer (29.9 mg)

Rt.=7.1 min, de 100%; LC-MS (ESI): m/z (M+1): 582.3 (Method 2)

¹H NMR (400 MHz, Methanol-d₄) δ ppm 8.11-8.24 (m, 2H), 7.88 (dd, J=6.5, 2.7 Hz, 1H), 7.75 (d, J=1.4 Hz, 1H), 7.50 (ddd, J=8.8, 4.2, 2.8 Hz, 1H), 7.27 (dd, J=10.2, 8.9 Hz, 1H), 7.05 (dd, J=5.6, 2.1 Hz, 1H), 5.87 (td, J=4.1, 2.1 Hz, 1H), 4.02-4.22 (m, 3H), 3.92 (td, J=8.3, 5.0 Hz, 1H), 2.94-3.07 (m, 1H), 2.74-2.87 (m, 1H), 2.32-2.68 (m, 8H), 2.32-2.50 (m, 4H), 2.30 (s, 3H), 2.14-2.25 (m, 2H).

Example 112 (Trans Enantiomer 1) was obtained as the first eluted diasteroisomer (2.4 mg)

Rt.=5.2 min, de 100%; LC-MS (ESI): m/z (M+1): 582.3 (Method 2)

¹H NMR (400 MHz, Methanol-d₄) δ ppm 8.22 (s, 1H), 8.17 (d, J=5.7 Hz, 1H), 7.90 (dd, J=6.5, 2.7 Hz, 1H), 7.78 (d, J=1.3 Hz, 1H), 7.45-7.56 (m, 1H), 7.27 (dd, J=10.2, 9.0 Hz, 1H), 7.04 (dd, J=5.7, 2.1 Hz, 1H), 5.87 (td, J=4.1, 2.2 Hz, 1H), 4.04-4.21 (m, 3H), 3.93 (td, J=8.3, 5.0 Hz, 1H), 3.15-3.24 (m, 1H), 3.07 (quin, J=7.8 Hz, 1H), 2.34-2.51 (m, 4H), 2.32-2.80 (m, 8H), 2.31 (s, 3H), 2.20-2.29 (m, 2H).

Second eluted couple Enantiomer 2 Cis/Trans mixture (48 mg) was further separated by chiral HPLC.

### Conditions:

| Column | Chiralpak AS-H (25 × 2.0 cm), 5 μ | |
|---|---|---|
| Mobile phase | n-Hexane/(Ethanol + 0.1% | |
| | isopropylamine) 75/25% v/v | |
| Flow rate (ml/min) | 17 | ml/min |
| DAD detection | 220 | nm |
| Loop | 750 | μL |

Example 113 (Cis Enantiomer 2) was obtained as the second eluted diasteroisomer (29.8 mg)

Rt.=9.9 min, de 99%; LC-MS (ESI): m/z (M+1): 582.3 (Method 2)

Example 114 (Trans Enantiomer 2) was obtained as the first eluted diasteroisomer (2.3 mg)

Rt.=7.7 min, de 100%; LC-MS (ESI): m/z (M+1): 582.3 (Method 2)

### Example 115: Cis N-(4-{[6-(5-chloro-2-fluorophenyl)-3-{[(2,2-dimethyl-1,3-dioxolan-4-yl)methyl]sulfanyl}pyridazin-4-yl]amino}pyridin-2-yl)-3-(4-methylpiperazin-1-yl)cyclobutane-1-carboxamide

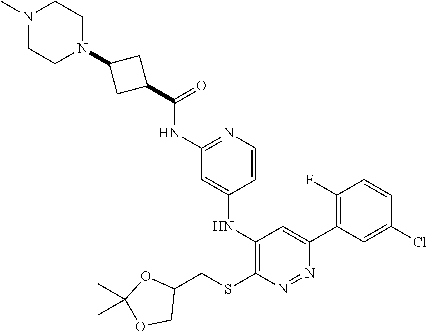

A mixture of 6-(5-chloro-2-fluorophenyl)-3-{[(2,2-dimethyl-1,3-dioxolan-4-yl)methyl]sulfanyl}pyridazin-4-amine (Intermediate 177, 60 mg, 0.16 mmol), N-(4-bromopyridin-2-yl)-3-(4-methylpiperazin-1-yl)cyclobutane-1-carboxamide (Intermediate 171, 63 mg, 0.18 mmol), K₃PO₄ (69 mg, 0.32 mmol), Pd₂(dba)₃ (15 mg, 0.02 mmol) and XantPhos (14 mg, 0.02 mmol) in 1,2-dimethoxyethane (2.2 mL) was degassed (N2/vacuum) then heated at 100° C. for 1 h. The mixture was diluted with EtOAc, filtered through a Celite® pad, washing with EtOAc. The filtrate was evaporated under vacuum. The crude material was purified by flash chromatography on Biotage silica NH cartridge (from cHex to 100% EtOAc), then it was sent to prep HPLC to afford title compound (25 mg, 0.04 mmol, 25% yield). Only the major isomer cis was isolated.

LC-MS (ESI): m/z (M+1): 642.3 (Method 2)

¹H NMR (500 MHz, Acetone-d₆) δ ppm 9.49 (s, 1H), 8.22 (d, J=1.2 Hz, 1H), 8.10-8.15 (m, 2H), 7.96 (s, 1H), 7.81 (d, J=1.1 Hz, 1H), 7.50-7.60 (m, 1H), 7.34 (dd, J=10.6, 8.9 Hz, 1H), 7.04 (dd, J=5.6, 2.1 Hz, 1H), 4.50 (quin, J=6.0 Hz, 1H), 4.16 (dd, J=8.5, 6.2 Hz, 1H), 3.84 (dd, J=8.4, 6.0 Hz, 1H), 3.67-3.77 (m, 1H), 3.59-3.67 (m, 1H), 3.08 (quin, J=8.6 Hz, 1H), 2.67 (quin, J=7.6 Hz, 1H), 2.29-2.36 (m, 2H), 2.22-2.55 (m, 8H), 2.18 (s, 3H), 2.12-2.17 (m, 2H), 1.40 (s, 3H), 1.30 (s, 3H).

### Example 116: N-(4-{[6-(5-chloro-2-fluorophenyl)-3-[(2,2-dimethyl-2H-1,3-benzodioxol-5-yl)methoxy]pyridazin-4-yl]amino}pyridin-2-yl)-3-(4-methylpiperazin-1-yl)propanamide

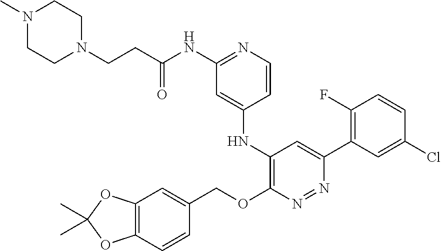 [SMILES: CN1CCN(CCC(=O)Nc2cc(Nc3cc(-c4cc(Cl)ccc4F)nnc3OCc3ccc4c(c3)OC(C)(C)O4)ccn2)CC1]

Example 116 was prepared following the procedure used for the synthesis of Example 1, starting from 6-(5-chloro-2-fluorophenyl)-3-[(2,2-dimethyl-2H-1,3-benzodioxol-5-yl)methoxy]pyridazin-4-amine (Intermediate 251, 120 mg, 0.30 mmol) and N-(4-bromopyridin-2-yl)-3-(4-methylpiperazin-1-yl)propanamide (Intermediate 2, 108 mg, 0.33 mmol) to afford title compound (22 mg, 0.03 mmol, 11% yield).

LC-MS (ESI): m/z (M+1): 648.3 (Method 2)

¹H NMR (400 MHz, Acetone-d₆) δ ppm10.78 (br s, 1H), 8.43-8.54 (m, 1H), 8.12-8.21 (m, 2H), 8.04 (dd, J=6.6, 2.6 Hz, 1H), 7.81-7.88 (m, 1H), 7.48-7.57 (m, 1H), 7.32 (dd, J=10.4, 8.9 Hz, 1H), 7.08 (dd, J=5.6, 2.1 Hz, 1H), 6.99-7.05 (m, 2H), 6.77 (d, J=8.6 Hz, 1H), 5.57 (s, 2H), 2.68-2.74 (m, 2H), 2.27-2.67 (m, 10H), 2.21 (s, 3H), 1.67 (s, 6H).

### Example 117 (trans) and Example 118 (cis): N-(4-{[6-(5-chloro-2-fluorophenyl)-3-[(3-hydroxycyclobutyl)methoxy]pyridazin-4-yl]amino}pyridin-2-yl)-3-(4-methylpiperazin-1-yl)cyclobutane-1-carboxamide

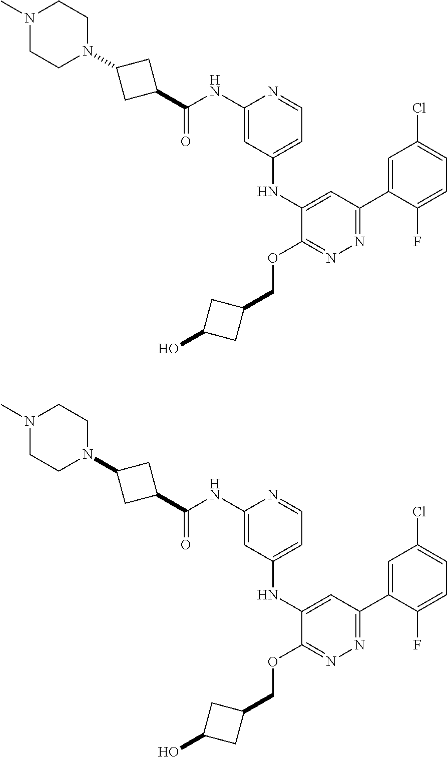

Diasteroisomeric mixture of cis and trans N-(4-{[6-(5-chloro-2-fluorophenyl)-3-[(3-hydroxycyclobutyl)methoxy]pyridazin-4-yl]amino}pyridin-2-yl)-3-(4-methylpiperazin-1-yl)cyclobutane-1-carboxamide (67 mg, 0.11 mmol, 100% yield) was prepared following the procedure used for the synthesis of Example 115, starting from Intermediate 157 (36 mg, 0.11 mmol) and Intermediate 171 (41 mg, 0.11 mmol).

The mixture was separated into the single diasteroisomers by preparative chiral HPLC.

### Conditions:

| Column | Chiralpak IC (25 × 2.0 cm), 5 μ | |
|---|---|---|
| Mobile phase | n-Hexane/(Ethanol + 0.1% | |
| | isopropylamine) 70/30% v/v | |
| Flow rate (ml/min) | 17 | ml/min |
| DAD detection | 220 | nm |
| Loop | 1250 | μL |

Example 117 (trans) was obtained as the first eluted diasteroisomer (1.6 mg)

Rt.=14.7 min, de >99.9%; LC-MS (ESI): m/z (M+1): 596.3 (Method 2)

¹H NMR (500 MHz, Chloroform-d) δ ppm 8.19 (d, J=5.6 Hz, 1H), 8.09-8.13 (m, 2H), 7.92-7.98 (m, 1H), 7.78 (d, J=1.4 Hz, 1H), 7.56 (br. s, 1H), 7.35-7.41 (m, 1H), 7.13 (dd, J=10.6, 8.8 Hz, 1H), 7.04 (dd, J=5.7, 2.1 Hz, 1H), 4.63 (d, J=5.1 Hz, 2H), 4.40 (quin, J=6.7 Hz, 1H), 2.99-3.20 (m, 2H), 2.62-2.71 (m, 2H), 2.43-2.61 (m, 3H), 2.36 (s, 3H), 2.21-2.35 (m, 2H), 2.05-2.79 (m, 8H), 1.96-2.04 (m, 2H).

Example 118 (cis) was obtained as the second eluted diasteroisomer (15 mg)

Rt.=16.3 min, de 99%; LC-MS (ESI): m/z (M+1): 596.3 (Method 2)

¹H NMR (400 MHz, Chloroform-d) δ ppm 8.82 (br. s, 1H), 8.19 (d, J=5.6 Hz, 1H), 8.10 (dd, J=6.6, 2.7 Hz, 1H), 8.06 (d, J=1.9 Hz, 1H), 7.76 (d, J=1.4 Hz, 1H), 7.59 (s, 1H), 7.37 (ddd, J=8.7, 4.2, 2.7 Hz, 1H), 7.13 (dd, J=10.6, 8.8 Hz, 1H), 7.03 (dd, J=5.7, 2.1 Hz, 1H), 4.62 (d, J=4.8 Hz, 2H), 4.39 (quin, J=6.7 Hz, 1H), 2.90 (quin, J=8.4 Hz, 1H), 2.76-2.84 (m, 1H), 2.61-2.70 (m, 2H), 2.49-2.59 (m, 1H), 2.41-2.49 (m, 2H), 2.33 (s, 3H), 2.20-2.29 (m, 2H), 2.17-2.77 (m, 8H), 1.95-2.04 (m, 2H).

### Example 119: Cis N-(4-{[6-(5-chloro-2-fluorophenyl)-3-[(3-hydroxyphenyl)methoxy]pyridazin-4-yl]amino}pyridin-2-yl)-3-(4-methylpiperazin-1-yl)cyclobutane-1-carboxamide

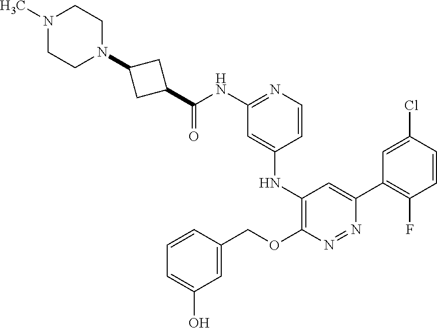

Example 119 was prepared following the procedure used for the synthesis of Example 115, starting from Intermediate 166 (50 mg, 0.13 mmol) and Intermediate 171 (55 mg, 0.16 mmol), heating 2 h 15 min at 110° C. under MW irradiation, to afford title compound (35 mg, 0.06 mmol, 44% yield). Only the major isomer cis was isolated.

LC-MS (ESI): m/z (M+1): 618.2 (Method 2)

¹H NMR (400 MHz, DMSO-d₆) δ ppm 10.36 (s, 1H), 9.47 (br. s, 1H), 9.13 (br. s, 1H), 8.17 (s, 1H), 8.14 (d, J=5.7 Hz, 1H), 7.95 (dd, J=6.6, 2.7 Hz, 1H), 7.71 (s, 1H), 7.58 (ddd, J=8.8, 4.1, 2.7 Hz, 1H), 7.41 (dd, J=10.4, 8.9 Hz, 1H), 7.14-7.23 (m, 1H), 7.05 (dd, J=5.6, 1.9 Hz, 1H), 6.92-7.00 (m, 2H), 6.72 (dd, J=8.0, 1.6 Hz, 1H), 5.61 (s, 2H), 2.91-3.06 (m, 1H), 2.56-2.65 (m, 1H), 2.14-2.21 (m, 2H), 2.14 (s, 3H), 2.10-2.44 (m, 8H), 1.92-2.04 (m, 2H).

### Example 120: N-(4-{[6-(5-chloro-2-fluorophenyl)-3-[(3-hydroxyphenyl)methoxy]pyridazin-4-yl]amino}pyridin-2-yl)-2-[(1S,4S)-5-methyl-2,5-diazabicyclo[2.2.1]heptan-2-yl]acetamide

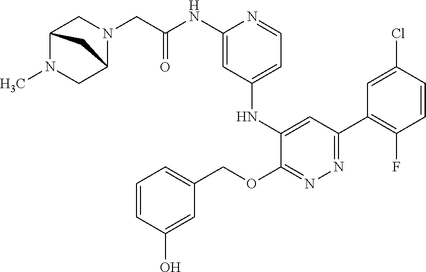 [SMILES: CN1C[C@@H]2C[C@H]1CN2CC(=O)Nc1cc(Nc2cc(-c3cc(Cl)ccc3F)nnc2OCc2cccc(O)c2)ccn1]

Example 120 was prepared following the procedure used for the synthesis of Example 2, starting from Intermediate 166 (50 mg, 0.13 mmol) and Intermediate 143 (47 mg, 0.14 mmol, to afford title compound (12 mg, 0.02 mmol, 16% yield).

LC-MS (ESI): m/z (M+1): 590.2 (Method 2)

¹H NMR (500 MHz, DMSO-d₆) δ ppm 9.76 (br. s, 1H), 9.05-9.65 (m, 2H), 8.05-8.23 (m, 2H), 7.92 (dd, J=6.5, 2.7 Hz, 1H), 7.69 (br. s, 1H), 7.57 (dt, J=8.6, 3.5 Hz, 1H), 7.36-7.44 (m, 1H), 7.18 (t, J=7.8 Hz, 1H), 7.08 (br. s, 1H), 6.89-6.99 (m, 2H), 6.72 (dd, J=8.0, 1.7 Hz, 1H), 5.60 (s, 2H), 3.31-3.34 (m, 1H), 3.30 (s, 2H), 3.17 (s, 1H), 2.77 (d, J=9.6 Hz, 1H), 2.65-2.70 (m, 1H), 2.58 (s, 2H), 2.27 (s, 3H), 1.58-1.69 (m, 2H).

### Example 121: N-(4-{[6-(5-chloro-2-fluorophenyl)-3-[(3-hydroxycyclobutyl)methoxy]pyridazin-4-yl]amino}pyridin-2-yl)-2-(4-methyl-1,4-diazepan-1-yl)acetamide

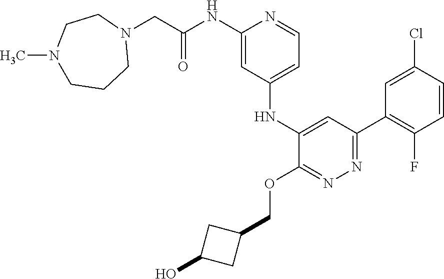

Example 121 was prepared following the procedure used for the synthesis of Example 1, starting from Intermediate 82 (84 mg, 0.26 mmol) and 3-({[4-amino-6-(5-chloro-2-fluorophenyl)pyridazin-3-yl]oxy}methyl)cyclobutan-1-ol (Intermediate 157, 80 mg, 0.25 mmol, to afford title compound (29 mg, 0.05 mmol, 21% yield).

LC-MS (ESI): m/z (M+1): 570.2 (Method 2)

¹H NMR (400 MHz, Methanol-d₄) δ ppm 8.22 (d, J=2.0 Hz, 1H), 8.19 (d, J=5.7 Hz, 1H), 7.86 (dd, J=6.5, 2.7 Hz, 1H), 7.76 (d, J=1.5 Hz, 1H), 7.49 (ddd, J=8.8, 4.2, 2.9 Hz, 1H), 7.23-7.32 (m, 1H), 7.09 (dd, J=5.7, 2.0 Hz, 1H), 4.61 (d, J=5.7 Hz, 2H), 4.17 (quin, J=7.3 Hz, 1H), 3.34 (br s, 2H), 2.85-2.95 (m, 4H), 2.74-2.83 (m, 4H), 2.43-2.59 (m, 3H), 2.40 (s, 3H), 1.80-1.98 (m, 4H).

### Example 122: N-(4-{[6-(5-chloro-2-fluorophenyl)-3-[(3-hydroxycyclobutyl)methoxy]pyridazin-4-yl]amino}pyridin-2-yl)-2-[(1S,4S)-5-methyl-2,5-diazabicyclo[2.2.1]heptan-2-yl]acetamide

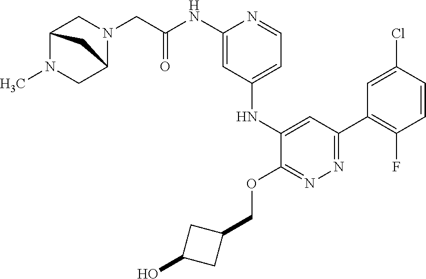

Example 122 was prepared following the procedure used for the synthesis of Example 1, starting from Intermediate 143 (95 mg, 0.27 mmol) and Intermediate 157 (80 mg, 0.23 mmol, to afford title compound (19 mg, 0.03 mmol, 14% yield). LC-MS (ESI): m/z (M+1): 568.2 (Method 2)

¹H NMR (500 MHz, Methanol-d₄) δ ppm 8.24 (d, J=1.9 Hz, 1H), 8.18 (d, J=5.8 Hz, 1H), 7.86 (dd, J=6.4, 2.7 Hz, 1H), 7.76 (d, J=1.6 Hz, 1H), 7.46-7.54 (m, 1H), 7.27 (dd, J=10.3, 8.9 Hz, 1H), 7.09 (dd, J=5.8, 2.2 Hz, 1H), 4.61 (d, J=5.8 Hz, 2H), 4.17 (quin, J=7.3 Hz, 1H), 3.44 (s, 1H), 3.40 (d, J=3.4 Hz, 2H), 3.37 (d, J=2.6 Hz, 1H), 2.91-2.99 (m, 1H), 2.84-2.91 (m, 2H), 2.72 (dd, J=10.4, 2.5 Hz, 1H), 2.40-2.46 (m, 4H), 2.39-2.56 (m, 2H), 1.80-1.92 (m, 4H).

### Example 123: N-(4-{[6-(5-chloro-2-fluorophenyl)-3-[(3-hydroxy-3-methylcyclobutyl)methoxy]pyridazin-4-yl]amino}pyridin-2-yl)-3-(4-methylpiperazin-1-yl)propanamide

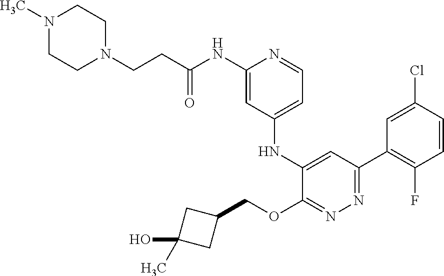

Example 123 was prepared following the procedure used for the synthesis of Example 1 starting from Intermediate 255 (147 mg, 0.43 mmol) and N-(4-bromopyridin-2-yl)-3-(4-methylpiperazin-1-yl)propanamide (Intermediate 2, 158 mg, 0.48 mmol) to afford title compound (22 mg, 0.04 mmol, 9% yield).

LC-MS (ESI): m/z (M+1): 584.2 (Method 2)

¹H NMR (500 MHz, DMSO-d₆) δ ppm 10.64 (s, 1H), 8.90 (s, 1H), 8.15 (d, J=5.8 Hz, 1H), 8.11 (s, 1H), 7.92 (dd, J=6.4, 2.7 Hz, 1H), 7.65 (s, 1H), 7.53-7.62 (m, 1H), 7.41 (dd, J=10.4, 9.0 Hz, 1H), 7.02 (dd, J=5.7, 2.1 Hz, 1H), 4.96 (s, 1H), 4.53 (d, J=6.6 Hz, 2H), 2.58-2.64 (m, 2H), 2.51-2.55 (m, 2H), 2.40 (td, J=15.3, 7.8 Hz, 1H), 2.20-2.48 (m, 8H), 2.14 (s, 3H), 2.05-2.13 (m, 2H), 1.83-1.95 (m, 2H), 1.26 (s, 3H).

### Example 124: Cis N-(4-{[6-(5-chloro-2-fluorophenyl)-3-[(3-hydroxy-3-methylcyclobutyl)methoxy]pyridazin-4-yl]amino}pyridin-2-yl)-3-(4-methylpiperazin-1-yl)cyclobutane-1-carboxamide

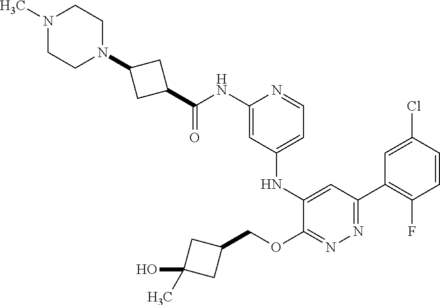

Example 124 was prepared following the procedure used for the synthesis of Example 115, starting from Intermediate 255 (83 mg, 0.25 mmol) and Intermediate 171 (95 mg, 0.27 mmol), to afford title compound (82 mg, 0.13 mmol, 55% yield). Only the major isomer cis was isolated. LC-MS (ESI): m/z (M+1): 610.3 (Method 2)

¹H NMR (400 MHz, Chloroform-d) δ ppm 8.82 (s, 1H), 8.19 (d, J=5.5 Hz, 1H), 8.11 (dd, J=6.6, 2.4 Hz, 1H), 8.06 (d, J=1.5 Hz, 1H), 7.76 (s, 1H), 7.50 (s, 1H), 7.33-7.41 (m, 1H), 7.08-7.18 (m, 1H), 7.01 (dd, J=5.6, 1.6 Hz, 1H), 4.64 (d, J=5.3 Hz, 2H), 2.90 (quin, J=8.3 Hz, 1H), 2.81 (quin, J=7.2 Hz, 1H), 2.49-2.62 (m, 1H), 2.33 (s, 3H), 2.30-2.74 (m, 12H), 2.20-2.29 (m, 2H), 2.10-2.19 (m, 2H), 1.49 (s, 3H).

### Example 125: methyl 3-({[6-(5-chloro-2-fluorophenyl)-4-({2-[3-(4-methylpiperazin-1-yl)propanamido]pyridin-4-yl}amino)pyridazin-3-yl]oxy}methyl)bicyclo[1.1.1]pentane-1-carboxylate

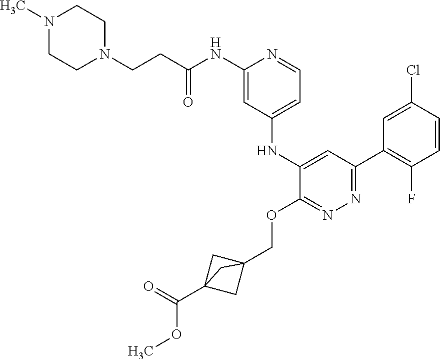 [SMILES: COC(=O)C12CC(COc3nnc(-c4cc(Cl)ccc4F)cc3Nc3ccnc(NC(=O)CCN4CCN(C)CC4)c3)(C1)C2]

Example 125 was prepared following the procedure used for the synthesis of Example 1 starting from Intermediate 259 (70 mg, 0.18 mmol) and Intermediate 2 (65 mg, 0.20 mmol) to afford title compound (40 mg, 0.06 mmol, 35% yield).

LC-MS (ESI): m/z (M+1): 624.3 (Method 2)

¹H NMR (500 MHz, Chloroform-d) δ ppm 11.25 (s, 1H), 8.25 (d, J=5.6 Hz, 1H), 8.06-8.13 (m, 2H), 7.77 (s, 1H), 7.31-7.41 (m, 1H), 7.13 (dd, J=10.2, 9.1 Hz, 1H), 6.95 (dd, J=5.5, 1.9 Hz, 1H), 6.82 (s, 1H), 4.76 (s, 2H), 3.69 (s, 3H), 2.73-2.81 (m, 2H), 2.53-2.59 (m, 2H), 2.47-2.82 (m, 8H), 2.37 (s, 3H), 2.16 (s, 6H).

### Example 126: Cis methyl 3-({[6-(5-chloro-2-fluorophenyl)-4-({2-[(1s,3s)-3-(4-methylpiperazin-1-yl)cyclobutaneamido]pyridin-4-yl}amino)pyridazin-3-yl]oxy}methyl)bicyclo[1.1.1]pentane-1-carboxylate

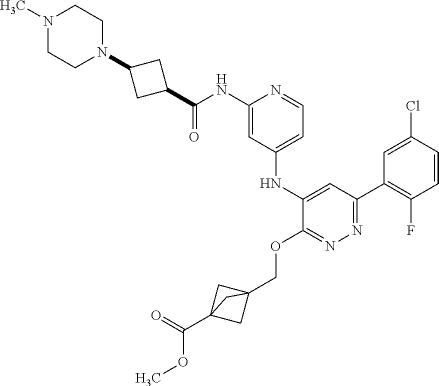

Example 126 was prepared following the procedure used for the synthesis of Example 115, starting from Intermediate 259 (80 mg, 0.21 mmol) and Intermediate 171 (82 mg, 0.23 mmol), to afford title compound (68 mg, 0.10 mmol, 49% yield). Only the major isomer cis was isolated. LC-MS (ESI): m/z (M+1): 650.3 (Method 2)

¹H NMR (400 MHz, Chloroform-d) δ ppm 8.88 (s, 1H), 8.22 (d, J=5.7 Hz, 1H), 8.04-8.15 (m, 2H), 7.77 (s, 1H), 7.37 (dt, J=8.3, 3.5 Hz, 1H), 7.09-7.18 (m, 1H), 6.98 (dd, J=5.6, 1.8 Hz, 1H), 6.83 (s, 1H), 4.76 (s, 2H), 3.70 (s, 3H), 2.92 (quin, J=8.3 Hz, 1H), 2.82 (quin, J=7.1 Hz, 1H), 2.42-2.51 (m, 2H), 2.54 (br. s, 8H), 2.34 (s, 3H), 2.21-2.29 (m, 2H), 2.16 (s, 6H).

### Example 127: N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)propanamide

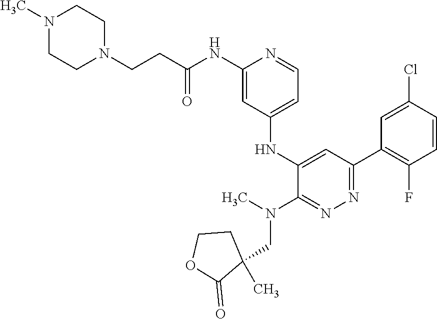 [SMILES: CN1CCN(CCC(=O)Nc2cc(Nc3cc(-c4cc(Cl)ccc4F)nnc3N(C)C[C@@]3(C)CCOC3=O)ccn2)CC1]

Example 127 was prepared following the procedure used for the synthesis of Example 1, starting from Intermediate 266 (153 mg, 0.42 mmol) and Intermediate 2 (152 mg, 0.46 mmol, to afford title compound (95 mg, 0.15 mmol, 37% yield).

LC-MS (ESI): m/z (M+1): 611.2 (Method 1)

¹H NMR (500 MHz, DMSO-d₆) δ ppm 10.57 (s, 1H), 8.72 (s, 1H), 8.09 (d, J=5.6 Hz, 1H), 8.00 (dd, J=6.7, 2.8 Hz, 1H), 7.90 (s, 1H), 7.65 (s, 1H), 7.53-7.60 (m, 1H), 7.41 (dd, J=10.6, 8.9 Hz, 1H), 6.78 (dd, J=5.6, 2.1 Hz, 1H), 4.18-4.32 (m, 2H), 3.99 (br. d, J=14.4 Hz, 1H), 3.56 (d, J=14.4 Hz, 1H), 2.96 (s, 3H), 2.56-2.62 (m, 2H), 2.49-2.54 (m, 2H), 2.21-2.47 (m, 9H), 2.13 (s, 3H), 1.94 (ddd, J=12.6, 7.1, 4.3 Hz, 1H), 1.12 (s, 3H).

### Example 128 (Enantiomer 1) and Example 129 (Enantiomer 2): N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)propanamide

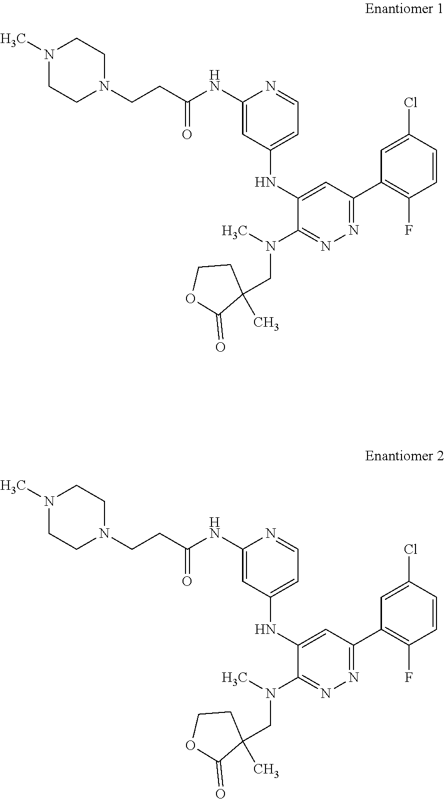 [SMILES: CN1CCN(CCC(=O)Nc2cc(Nc3cc(-c4cc(Cl)ccc4F)nnc3N(C)CC3(C)CCOC3=O)ccn2)CC1 ; CN1CCN(CCC(=O)Nc2cc(Nc3cc(-c4cc(Cl)ccc4F)nnc3N(C)CC3(C)CCOC3=O)ccn2)CC1]

Racemate N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)propanamide (Example 127, 86 mg) was separated into the single enantiomers by preparative chiral HPLC.

### Conditions:

| Column | Chiralcel OD-H (25 × 3.0 cm), 5 μ | |
|---|---|---|
| Mobile phase | n-Hexane/(Ethanol + 0.1% | |
| | isopropylamine) 70/30% v/v | |
| Flow rate (ml/min) | 38 | ml/min |
| DAD detection | 220 | nm |
| Loop | 900 | μL |

Example 128 was obtained as first eluted enantiomer (33 mg)

Rt.=12.2 min, ee >99.9%; LC-MS (ESI): m/z (M+1): 611.2 (Method 1)

¹H NMR (400 MHz, Chloroform-d) δ ppm 11.05-11.20 (m, 1H), 8.24 (d, J=5.5 Hz, 1H), 8.21 (d, J=2.0 Hz, 1H), 8.19 (dd, J=6.6, 2.9 Hz, 1H), 7.89 (d, J=1.3 Hz, 1H), 7.54 (s, 1H), 7.38 (ddd, J=8.8, 4.20, 2.9 Hz, 1H), 7.15 (dd, J=10. 6, 8.8 Hz, 1H), 6.91 (dd, J=5.6, 2.1 Hz, 1H), 4.31 (t, J=7.2 Hz, 2H), 3.78-3.90 (m, 2H), 2.94 (s, 3H), 2.75-2.82 (m, 2H), 2.54-2.75 (m, 10H), 2.39 (s, 3H), 2.18-2.30 (m, 1H), 1.99-2.09 (m, 1H), 1.26 (s, 3H).

Example 129 was obtained as the second eluted enantiomer (32 mg)

Rt.=16.1 min, ee 98%; LC-MS (ESI): m/z (M+1): 611.2 (Method 1)

¹H NMR (400 MHz, Chloroform-d) δ ppm 11.05-11.20 (m, 1H), 8.24 (d, J=5.5 Hz, 1H), 8.21 (d, J=2.0 Hz, 1H), 8.19 (dd, J=6.6, 2.9 Hz, 1H), 7.89 (d, J=1.3 Hz, 1H), 7.54 (s, 1H), 7.38 (ddd, J=8.8, 4.2, 2.9 Hz, 1H), 7.15 (dd, J=10. 6, 8.8 Hz, 1H), 6.91 (dd, J=5.6, 2.1 Hz, 1H), 4.31 (t, J=7.2 Hz, 2H), 3.78-3.90 (m, 2H), 2.94 (s, 3H), 2.75-2.82 (m, 2H), 2.54-2.75 (m, 10H), 2.39 (s, 3H), 2.18-2.30 (m, 1H), 1.99-2.09 (m, 1H), 1.26 (s, 3H).

### Example 130: N-(4-{[6-(5-chloro-2-fluorophenyl)-3-[methyl(4,4,4-trifluoro-3-hydroxybutyl)amino]pyridazin-4-yl]amino}pyridin-2-yl)-2-[(1S,4S)-5-methyl-2,5-diazabicyclo[2.2.1]heptan-2-yl]acetamide

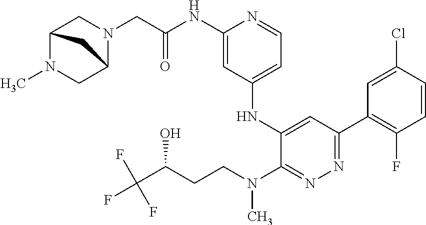 [SMILES: CN(CC[C@@H](O)C(F)(F)F)c1nnc(-c2cc(Cl)ccc2F)cc1Nc1ccnc(NC(=O)CN2C[C@@H]3C[C@H]2CN3C)c1]

Example 130 was prepared following the procedure used for the synthesis of Example 1, starting from Intermediate 247 (45 mg, 0.12 mmol) and Intermediate 143 (46 mg, 0.13 mmol) to afford title compound (25 mg, 0.04 mmol, 34% yield).

LC-MS (ESI): m/z (M+1): 623.3 (Method 2)

¹H NMR (400 MHz, DMSO-d₆) δ ppm 9.72 (s, 1H), 8.81 (br. s, 1H), 8.08 (d, J=5.6 Hz, 1H), 7.99 (dd, J=6.6, 2.7 Hz, 1H), 7.94 (s, 1H), 7.67 (s, 1H), 7.56 (dt, J=8.7, 3.5 Hz, 1H), 7.37-7.44 (m, 1H), 6.87 (br. d, J=4.5 Hz, 1H), 6.18 (d, J=5.6 Hz, 1H), 4.04 (br. s, 1H), 3.41-3.62 (m, 2H), 3.30-3.34 (m, 1H), 3.29 (s, 2H), 3.17 (s, 1H), 2.91 (s, 3H), 2.76 (d, J=9.5 Hz, 1H), 2.68 (dd, J=9.3, 2.2 Hz, 1H), 2.58 (s, 2H), 2.27 (s, 3H), 1.85-1.98 (m, 1H), 1.67-1.78 (m, 1H), 1.59-1.68 (m, 2H)

### Example 131 (Diasteroisomer 1) and Example 132 (Diasteroisomer 2): N-(4-{[6-(5-chloro-2-fluorophenyl)-3-[methyl(4,4,4-trifluoro-3-hydroxybutyl)amino]pyridazin-4-yl]amino}pyridin-2-yl)-2-[(1S,4S)-5-methyl-2,5-diazabicyclo[2.2.1]heptan-2-yl]acetamide

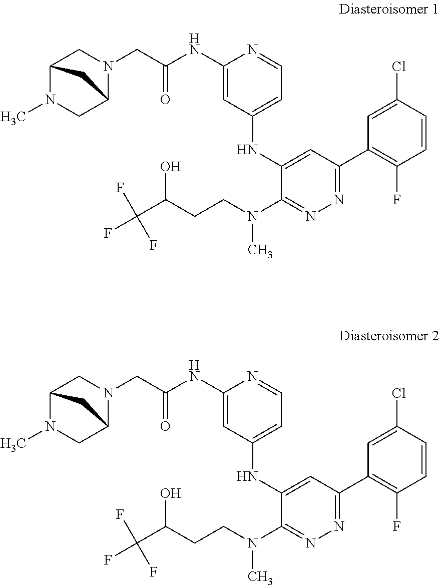 [SMILES: CN(CCC(O)C(F)(F)F)c1nnc(-c2cc(Cl)ccc2F)cc1Nc1ccnc(NC(=O)CN2C[C@@H]3C[C@H]2CN3C)c1 ; CN(CCC(O)C(F)(F)F)c1nnc(-c2cc(Cl)ccc2F)cc1Nc1ccnc(NC(=O)CN2C[C@@H]3C[C@H]2CN3C)c1]

Diasteroisomeric mixture of Example 130 (22 mg) was separated into the single diasteroisomers by preparative chiral HPLC.

### Conditions:

| Column | Chiralpak AD-H (25 × 3.0 cm), 5 μ | |
|---|---|---|
| Mobile phase | n-Hexane/(Ethanol + 0.1% | |
| | isopropylamine) 70/30% v/v | |
| Flow rate (ml/min) | 39 | ml/min |
| DAD detection | 220 | nm |
| Loop | 850 | μL |

Example 131 was obtained as first eluted diasteroisomer (33 mg)

Rt.=19.9 min, de >99.9%; LC-MS (ESI): m/z (M+1): 623.3 (Method 2)

¹H NMR (400 MHz, DMSO-d₆) δ ppm 9.72 (s, 1H), 8.81 (br. s, 1H), 8.08 (d, J=5.6 Hz, 1H), 7.99 (dd, J=6.6, 2.7 Hz, 1H), 7.94 (s, 1H), 7.67 (s, 1H), 7.56 (dt, J=8.7, 3.5 Hz, 1H), 7.37-7.44 (m, 1H), 6.87 (br. d, J=4.5 Hz, 1H), 6.18 (d, J=5.6 Hz, 1H), 4.04 (br. s, 1H), 3.41-3.62 (m, 2H), 3.30-3.34 (m, 1H), 3.29 (s, 2H), 3.17 (s, 1H), 2.91 (s, 3H), 2.76 (d, J=9.5 Hz, 1H), 2.68 (dd, J=9.3, 2.2 Hz, 1H), 2.58 (s, 2H), 2.27 (s, 3H), 1.85-1.98 (m, 1H), 1.67-1.78 (m, 1H), 1.59-1.68 (m, 2H)

Example 132 was obtained as the second eluted diasteroisomer (32 mg)

Rt.=26.5 min, de >99.9%; LC-MS (ESI): m/z (M+1): 623.3 (Method 2)

¹H NMR (400 MHz, DMSO-d₆) δ ppm 9.72 (s, 1H), 8.81 (br. s, 1H), 8.08 (d, J=5.6 Hz, 1H), 7.99 (dd, J=6.6, 2.7 Hz, 1H), 7.94 (s, 1H), 7.67 (s, 1H), 7.56 (dt, J=8.7, 3.5 Hz, 1H), 7.37-7.44 (m, 1H), 6.87 (br. d, J=4.5 Hz, 1H), 6.18 (d, J=5.6 Hz, 1H), 4.04 (br. s, 1H), 3.41-3.62 (m, 2H), 3.30-3.34 (m, 1H), 3.29 (s, 2H), 3.17 (s, 1H), 2.91 (s, 3H), 2.76 (d, J=9.5 Hz, 1H), 2.68 (dd, J=9.3, 2.2 Hz, 1H), 2.58 (s, 2H), 2.27 (s, 3H), 1.85-1.98 (m, 1H), 1.67-1.78 (m, 1H), 1.59-1.68 (m, 2H)

### Example 133: methyl 4-[6-(5-chloro-2-fluorophenyl)-4-({2-[3-(4-methylpiperazin-1-yl)propanamido]pyridin-4-yl}amino)pyridazin-3-yl]morpholine-2-carboxylate

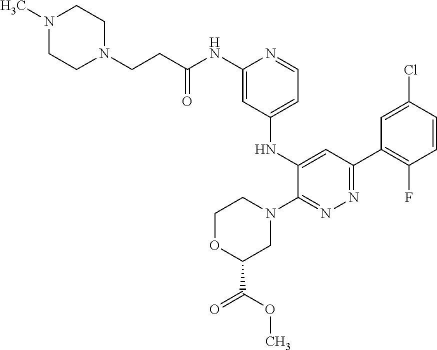 [SMILES: COC(=O)[C@H]1CN(c2nnc(-c3cc(Cl)ccc3F)cc2Nc2ccnc(NC(=O)CCN3CCN(C)CC3)c2)CCO1]

Example 133 was prepared following the procedure used for the synthesis of Example 1, starting from Intermediate 270 (60 mg, 0.16 mmol) and Intermediate 2 (70 mg, 0.21 mmol) to afford title compound (28 mg, 0.05 mmol, 28% yield).

LC-MS (ESI): m/z (M+1): 613.2 (Method 2)

¹H NMR (400 MHz, Chloroform-d) δ ppm 11.13 (s, 1H), 8.21-8.29 (m, 2H), 8.17 (dd, J=6.6, 2.7 Hz, 1H), 7.90 (br. s, 1H), 7.87 (s, 1H), 7.33-7.41 (m, 1H), 7.13 (dd, J=10.3, 9.0 Hz, 1H), 7.02 (dd, J=5.7, 1.8 Hz, 1H), 4.54 (t, J=3.1 Hz, 1H), 4.07 (dt, J=11.8, 3.2 Hz, 1H), 3.98 (s, 3H), 3.91-3.96 (m, 1H), 3.84-3.92 (m, 1H), 3.65 (ddd, J=12.8, 9.8, 3.0 Hz, 1H), 3.40 (br. d, J=13.3 Hz, 1H), 3.19 (dd, J=12.7, 2.6 Hz, 1H), 2.74-2.79 (m, 2H), 2.54-2.59 (m, 2H), 2.62 (br. s, 8H), 2.37 (s, 3H).

### Example 134: 4-[6-(5-chloro-2-fluorophenyl)-4-({2-[3-(4-methylpiperazin-1-yl)propanamido]pyridin-4-yl}amino)pyridazin-3-yl]morpholine-2-carboxylate lithium salt

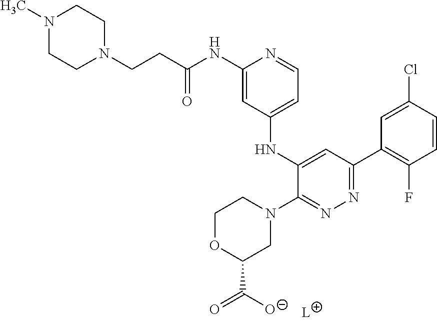 [SMILES: CN1CCN(CCC(=O)Nc2cc(Nc3cc(-c4cc(Cl)ccc4F)nnc3N3CCO[C@@H](C(=O)[O-])C3)ccn2)CC1]

Example 134 was prepared following the procedure used for the synthesis of Example 99, starting from Example 133 (18 mg, 0.03 mmol) to afford title compound (17 mg, 0.03 mmol, 98% yield). LC-MS (ESI): m/z (M+1): 599.2 (Method 2)

¹H NMR (400 MHz, DMSO-d) 6 ppm 11.61 (s, 1H), 10.55 (s, 1H), 8.21 (s, 1H), 8.13 (d, J=5.5 Hz, 1H), 7.97 (dd, J=6.6, 2.9 Hz, 1H), 7.66 (s, 1H), 7.53-7.58 (m, 1H), 7.34-7.42 (m, 1H), 7.15 (d, J=5.9 Hz, 1H), 4.03-4.10 (m, 1H), 3.84-3.94 (m, 2H), 3.60-3.70 (m, 1H), 2.58-2.70 (m, 4H), 2.25-2.47 (m, 12H), 2.14 (m, 3H).

### Example 135 (Enantiomer 1) and Example 136 (Enantiomer 2): Cis N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)cyclobutane-1-carboxamide

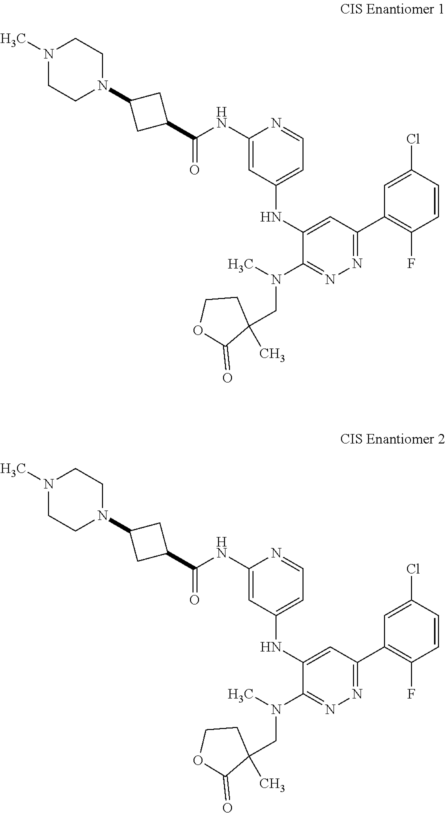

Racemic mixture of N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)cyclobutane-1-carboxamide (173 mg, 0.27 mmol, 74% yield) was prepared following the procedure used for the synthesis of Example 1, starting from Intermediate 266 (133 mg, 0.36 mmol) and Intermediate 171 (137 mg, 0.39 mmol). Only the major isomer cis was isolated. Then it was separated into the single enantiomers by preparative chiral HPLC.

### Conditions:

| Column | Chiralcel OD-H (25 × 2.0 cm), 5 μ | |
|---|---|---|
| Mobile phase | n-Hexane/(Ethanol + 0.1% | |
| | isopropylamine) 75/25% v/v | |
| Flow rate (ml/min) | 17 | ml/min |
| DAD detection | 220 | nm |
| Loop | 1000 | μL |

Example 135 was obtained as first eluted enantiomer (62 mg)

Rt.=11.5 min, ee >99.9%; LC-MS (ESI): m/z (M+1): 637.3 (Method 2)

¹H NMR (500 MHz, Chloroform-d) δ ppm 8.79 (s, 1H), 8.21 (d, J=1.5 Hz, 1H), 8.16-8.19 (m, 2H), 7.87 (d, J=1.1 Hz, 1H), 7.57 (s, 1H), 7.34-7.40 (m, 1H), 7.14 (dd, J=10.4, 8.8 Hz, 1H), 6.92 (dd, J=5.6, 2.2 Hz, 1H), 4.30 (t, J=7.1 Hz, 2H), 3.74-3.88 (m, 2H), 2.92 (s, 3H), 2.85-2.96 (m, 1H), 2.81 (quin, J=7.2 Hz, 1H), 2.39-2.50 (m, 2H), 2.33 (s, 3H), 2.19-2.73 (m, 8H), 2.18-2.30 (m, 3H), 2.03 (dt, J=13.1, 7.1 Hz, 1H), 1.24 (s, 3H).

Example 136 was obtained as the second eluted enantiomer (63 mg)

Rt.=14.3 min, ee 90%; LC-MS (ESI): m/z (M+1): 637.3 (Method 2)

### Example 137: N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)propanamide

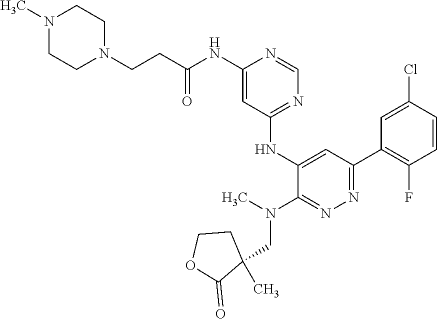 [SMILES: CN1CCN(CCC(=O)Nc2cc(Nc3cc(-c4cc(Cl)ccc4F)nnc3N(C)C[C@@]3(C)CCOC3=O)ncn2)CC1]

Example 137 was prepared following the procedure used for the synthesis of Example 115, starting from Intermediate 266 (160 mg, 0.44 mmol) and Intermediate 272 (140 mg, 0.49 mmol, to afford title compound (185 mg, 0.30 mmol, 69% yield).

LC-MS (ESI): m/z (M+1): 612.3 (Method 2)

¹H NMR (500 MHz, Chloroform-d) δ ppm 11.56 (s, 1H), 9.07 (d, J=1.5 Hz, 1H), 8.61 (s, 1H), 8.21 (s, 1H), 8.14 (dd, J=6.6, 2.7 Hz, 1H), 7.86 (s, 1H), 7.35-7.42 (m, 1H), 7.16 (dd, J=10.3, 8.9 Hz, 1H), 4.23-4.35 (m, 2H), 3.70-3.84 (m, 2H), 2.91 (s, 3H), 2.73-2.78 (m, 2H), 2.54-2.59 (m, 2H), 2.62 (br. s, 8H), 2.38 (s, 3H), 2.25 (ddd, J=13.1, 7.8, 6.7 Hz, 1H), 2.00 (ddd, J=13.2, 7.5, 6.2 Hz, 1H), 1.26 (s, 3H).

### Example 138 (Enantiomer 1) and Example 139 (Enantiomer 2): N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)propanamide

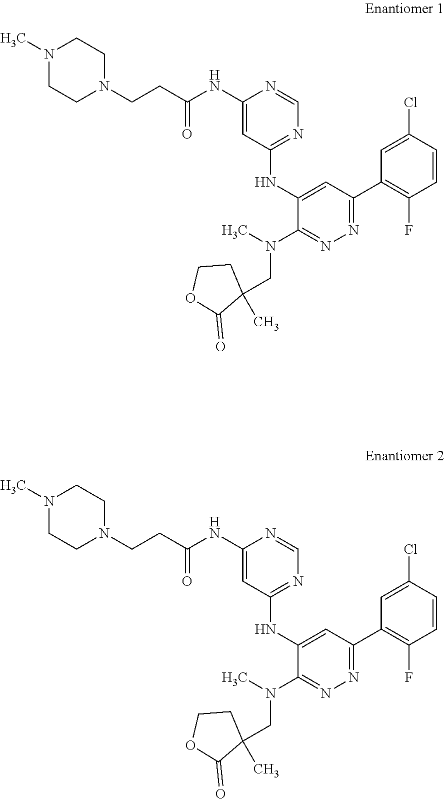 [SMILES: CN1CCN(CCC(=O)Nc2cc(Nc3cc(-c4cc(Cl)ccc4F)nnc3N(C)CC3(C)CCOC3=O)ncn2)CC1 ; CN1CCN(CCC(=O)Nc2cc(Nc3cc(-c4cc(Cl)ccc4F)nnc3N(C)CC3(C)CCOC3=O)ncn2)CC1]

Racemate N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)propanamide (Example 137, 180 mg) was separated into the single enantiomers by preparative chiral HPLC.

### Conditions:

| Column | Chiralpak IA (25 × 3.0 cm), 5 μ | |
|---|---|---|
| Mobile phase | n-Hexane/(Ethanol/Methanol 1/1 + 0.1% | |
| | isopropylamine) 50/50% v/v | |
| Flow rate (ml/min) | 38 | ml/min |
| DAD detection | 220 | nm |
| Loop | 700 | μL |

Example 138 was obtained as first eluted enantiomer (70 mg)

Rt.=11.5 min, ee >99.9%; LC-MS (ESI): m/z (M+1): 612.3 (Method 2)

¹H NMR (500 MHz, Chloroform-d) δ ppm 11.56 (s, 1H), 9.07 (d, J=1.5 Hz, 1H), 8.61 (s, 1H), 8.21 (s, 1H), 8.14 (dd, J=6.6, 2.7 Hz, 1H), 7.86 (s, 1H), 7.35-7.42 (m, 1H), 7.16 (dd, J=10.3, 8.9 Hz, 1H), 4.23-4.35 (m, 2H), 3.70-3.84 (m, 2H), 2.91 (s, 3H), 2.73-2.78 (m, 2H), 2.54-2.59 (m, 2H), 2.62 (br. s, 8H), 2.38 (s, 3H), 2.25 (ddd, J=13.1, 7.8, 6.7 Hz, 1H), 2.00 (ddd, J=13.2, 7.5, 6.2 Hz, 1H), 1.26 (s, 3H).

Example 139 was obtained as the second eluted enantiomer (71 mg)

Rt.=15.6 min, ee 95.8%; LC-MS (ESI): m/z (M+1): 612.3 (Method 2)

### Example 140: N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide

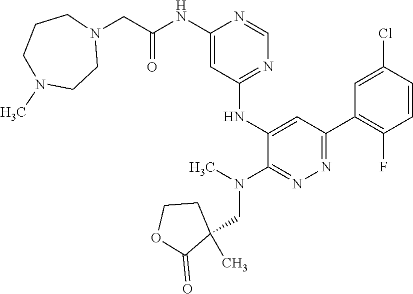 [SMILES: CN1CCCN(CC(=O)Nc2cc(Nc3cc(-c4cc(Cl)ccc4F)nnc3N(C)C[C@@]3(C)CCOC3=O)ncn2)CC1]

Example 140 was prepared following the procedure used for the synthesis of Example 1, starting from Intermediate 266 (112 mg, 0.31 mmol) and Intermediate 82 (111 mg, 0.34 mmol, to afford title compound (53 mg, 0.09 mmol, 28% yield).

LC-MS (ESI): m/z (M+1): 613.3 (Method 2)

¹H NMR (400 MHz, Chloroform-d) δ ppm 9.74 (s, 1H), 8.26 (d, J=2.0 Hz, 1H), 8.24 (d, J=5.7 Hz, 1H), 8.17 (dd, J=6.6, 2.6 Hz, 1H), 7.88 (s, 1H), 7.63 (s, 1H), 7.31-7.44 (m, 1H), 7.14 (dd, J=10.4, 8.9 Hz, 1H), 6.94 (dd, J=5.6, 2.1 Hz, 1H), 4.30 (t, J=7.1 Hz, 2H), 3.82 (s, 2H), 3.32 (s, 2H), 2.92 (s, 3H), 2.85-2.93 (m, 4H), 2.65-2.75 (m, 4H), 2.41 (s, 3H), 2.15-2.28 (m, 1H), 1.98-2.10 (m, 1H), 1.90 (quin, J=5.9 Hz, 2H), 1.24 (s, 3H).

### Example 141 (Enantiomer 1) and Example 142 (Enantiomer 2): N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide

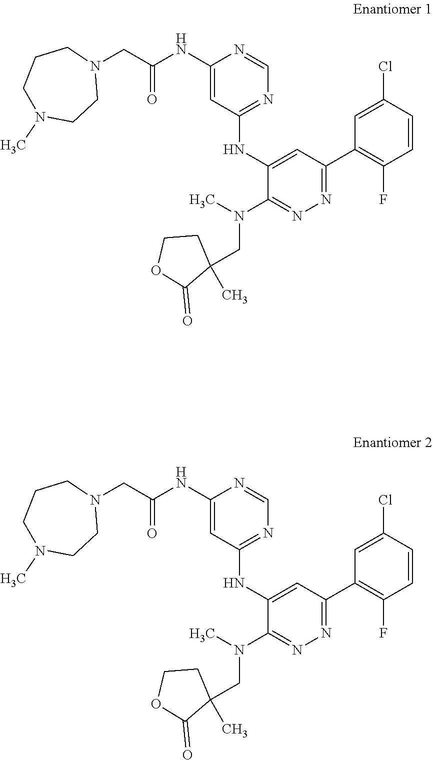 [SMILES: CN1CCCN(CC(=O)Nc2cc(Nc3cc(-c4cc(Cl)ccc4F)nnc3N(C)CC3(C)CCOC3=O)ncn2)CC1 ; CN1CCCN(CC(=O)Nc2cc(Nc3cc(-c4cc(Cl)ccc4F)nnc3N(C)CC3(C)CCOC3=O)ncn2)CC1]

Racemate N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide (Example 140, 48 mg) was separated into the single enantiomers by preparative chiral HPLC.

### Conditions:

| Column | Chiralcel OD-H (25 × 2.0 cm), 5 μ | |
|---|---|---|
| Mobile phase | n-Hexane/(Ethanol/Methanol 1/1 + 0.1% | |
| | isopropylamine) 40/60% v/v | |
| Flow rate (ml/min) | 17 | ml/min |
| DAD detection | 220 | nm |
| Loop | 1500 | μL |

Example 141 was obtained as first eluted enantiomer (20.6 mg)

Rt.=7.6 min, ee >99.9%; LC-MS (ESI): m/z (M+1): 613.3 (Method 2)

Example 142 was obtained as the second eluted enantiomer (21.8 mg)

Rt.=12.0 min, ee 99.9%; LC-MS (ESI): m/z (M+1): 613.3 (Method 2)

### Example 143 (Enantiomer 1) and Example 144 (Enantiomer 2): Cis N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)cyclobutane-1-carboxamide

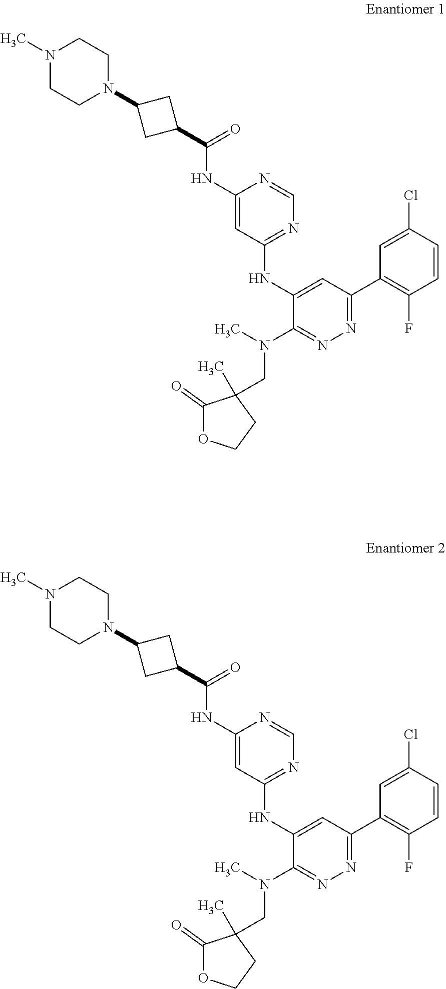

Racemate cis N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)cyclobutane-1-carboxamide (236 mg, 0.37 mmol, 71% yield) was prepared following the procedure used for the synthesis of Example 115, starting from Intermediate 266 (190 mg, 0.52 mmol) and Intermediate 190 (171 mg, 0.55 mmol). Then it was separated into the single enantiomers by preparative chiral HPLC.

### Conditions:

| Column | Chiralpak IC (25 × 3.0 cm), 5 μ | |
|---|---|---|
| Mobile phase | n-Hexane/(Ethanol + 0.1% | |
| | isopropylamine) 65/35% v/v | |
| Flow rate (ml/min) | 37 | ml/min |
| DAD detection | 220 | nm |
| Loop | 400 | μL |

Example 143 was obtained as first eluted enantiomer (60.7 mg)

Rt.=22.3 min, ee >99.9%; LC-MS (ESI): m/z (M+1): 638.3 (Method 4)

¹H NMR (400 MHz, Chloroform-d) δ ppm 9.64 (s, 1H), 9.10 (s, 1H), 8.60 (s, 1H), 8.29 (s, 1H), 8.14 (dd, J=6.6, 2.6 Hz, 1H), 7.89 (s, 1H), 7.36-7.43 (m, 1H), 7.12-7.20 (m, 1H), 4.22-4.39 (m, 2H), 3.68-3.88 (m, 2H), 2.93-3.04 (m, 1H), 2.91 (s, 3H), 2.84 (quin, J=6.6 Hz, 1H), 2.41-2.74 (m, 10H), 2.36 (s, 3H), 2.17-2.30 (m, 3H), 1.94-2.10 (m, 1H), 1.26 (s, 3H).

Example 144 was obtained as the second eluted enantiomer (50.4 mg)

Rt.=24.0 min, ee 87.2%; LC-MS (ESI): m/z (M+1): 638.3 (Method 4)

### Example 145: ethyl 3-{[6-(5-chloro-2-fluorophenyl)-4-({2-[3-(4-methylpiperazin-1-yl)propanamido]pyridin-4-yl}amino)pyridazin-3-yl](methyl)amino}-2,2-dimethylpropanoate

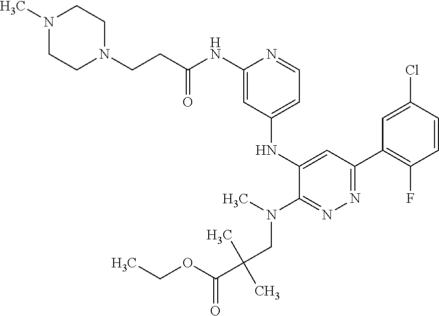 [SMILES: CCOC(=O)C(C)(C)CN(C)c1nnc(-c2cc(Cl)ccc2F)cc1Nc1ccnc(NC(=O)CCN2CCN(C)CC2)c1]

Example 145 was prepared following the procedure used for the synthesis of Example 1, starting from Intermediate 279 (70 mg, 0.18 mmol) and Intermediate 2 (67 mg, 0.20 mmol, to afford title compound (36 mg, 0.06 mmol, 31% yield).

LC-MS (ESI): m/z (M+1): 627.5 (Method 2)

¹H NMR (500 MHz, Chloroform-d) δ ppm 11.13 (s, 1H), 8.23 (d, J=5.5 Hz, 1H), 8.15-8.20 (m, 2H), 7.85 (s, 1H), 7.52 (s, 1H), 7.33-7.41 (m, 1H), 7.13 (dd, J=10.2, 9.0 Hz, 1H), 6.91 (dd, J=5.6, 2.0 Hz, 1H), 4.15 (q, J=7.1 Hz, 2H), 3.69 (s, 2H), 2.85 (s, 3H), 2.76 (t, J=5.9 Hz, 2H), 2.54-2.58 (m, 2H), 2.45-2.80 (m, 8H), 2.37 (s, 3H), 1.23 (t, J=7.1 Hz, 3H), 1.18 (s, 6H).

### Example 146 (cis Enantiomer 1) and Example 147 (trans Enantiomer 1): N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)cyclobutane-1-carboxamide

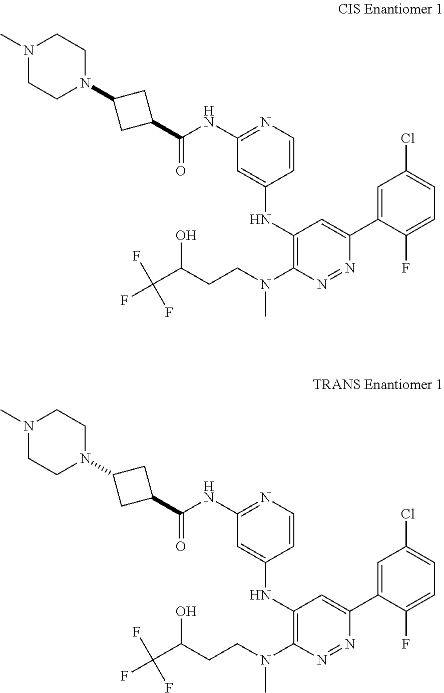

Diasteroisomeric mixture of cis and trans N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)cyclobutane-1-carboxamide was prepared following the procedure used for the synthesis of Example 115, starting from Enantiomer 1 4-{[4-amino-6-(5-chloro-2-fluorophenyl)pyridazin-3-yl](methyl)amino}-1,1,1-trifluorobutan-2-ol (Intermediate 280, 76 mg, 0.20 mmol) and Intermediate 171 (78 mg, 0.22 mmol). The crude material was purified by flash chromatography on Biotage silica NH cartridge (from cHex to 80% EtOAc), to afford cis diasteroisomer (Example 146, 75 mg, 0.12 mmol, 58% yield) and a cis and trans mixture that was sent to prep HPLC to afford trans diasteroisomer (Example 147, 5 mg, 0.01 mmol, 5% yield).

### Preparative Chiral HPLC Conditions:

| Column | Chiralpak AD-H (25 × 2.0 cm), 5 μ | |
|---|---|---|
| Mobile phase | n-Hexane/(Ethanol + 0.1% | |
| | isopropylamine) 80/20% v/v | |
| Flow rate (ml/min) | 17 | ml/min |
| DAD detection | 220 | nm |
| Loop | 800 | μL |

Example 146 (cis Enantiomer 1) was the second eluted diasteroisomer

Rt.=15.9 min. de 99.0%; LC-MS (ESI): m/z (M+1): 651.4 (Method 2)

¹H NMR (400 MHz, Chloroform-d) δ ppm 8.84 (br. s, 1H), 8.12-8.21 (m, 2H), 7.93-7.99 (m, 1H), 7.79 (s, 1H), 7.37 (dt, J=8.6, 3.5 Hz, 1H), 7.30 (br. s, 1H), 7.08-7.16 (m, 1H), 6.95 (dd, J=5.6, 1.8 Hz, 1H), 4.54 (br. s, 1H), 4.19-4.31 (m, 1H), 3.57-3.71 (m, 1H), 3.22-3.35 (m, 1H), 2.98 (s, 3H), 2.90 (quin, J=8.4 Hz, 1H), 2.80 (quin, J=7.2 Hz, 1H), 2.35-2.72 (m, 10H), 2.33 (s, 3H), 2.15-2.29 (m, 3H), 1.87-2.03 (m, 1H).

Example 147 (trans Enantiomer 1) was the first eluted diasteroisomer

Rt.=11.5 min, de >99.9%; LC-MS (ESI): m/z (M+1): 651.4 (Method 2)

¹H NMR (500 MHz, Chloroform-d) δ ppm 8.16-8.21 (m, 2H), 8.02 (br. s, 1H), 7.90 (s, 1H), 7.79-7.83 (m, 1H), 7.35-7.41 (m, 1H), 7.27 (br. s, 1H), 7.12 (dd, J=10.6, 8.9 Hz, 1H), 6.98 (dd, J=5.6, 2.1 Hz, 1H), 4.11-4.35 (m, 2H), 3.63 (ddd, J=13.6, 9.2, 6.2 Hz, 1H), 3.27-3.38 (m, 1H), 3.02-3.13 (m, 2H), 2.99 (s, 3H), 2.33 (s, 3H), 2.26-2.81 (m, 12H), 2.15-2.25 (m, 1H), 1.95 (dddd, J=14.6, 10.5, 6.1, 4.0 Hz, 1H).

### Example 148 (trans Enantiomer 2) and Example 149 (cis Enantiomer 2): N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)cyclobutane-1-carboxamide

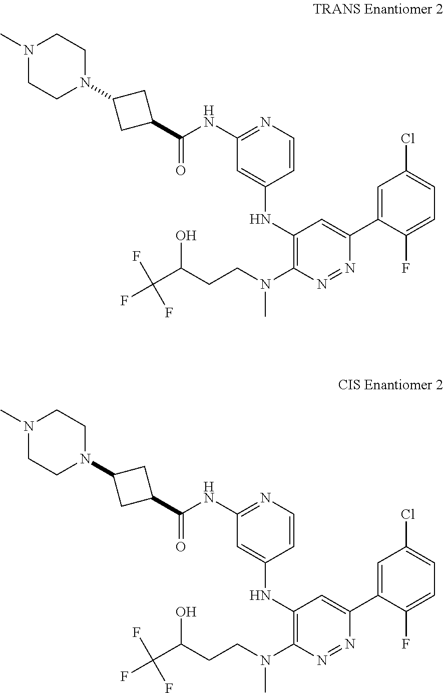

Diasteroisomeric mixture of cis and trans N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)cyclobutane-1-carboxamide (132 mg, 0.2 mmol, 98% yield) was prepared following the procedure used for the synthesis of Example 115, starting from Enantiomer 2 4-{[4-amino-6-(5-chloro-2-fluorophenyl)pyridazin-3-yl](methyl)amino}-1,1,1-trifluorobutan-2-ol (Intermediate 281, 78 mg, 0.21 mmol) and Intermediate 171 (80 mg, 0.23 mmol). It was separated into the single diasteroisomers by preparative chiral HPLC.

### Conditions:

| Column | Chiralpak AD-H (25 × 3.0 cm), 5μ | |
|---|---|---|
| Mobile phase | n-Hexane/(Ethanol + 0.1% | |
| | isopropylamine) 80/20% v/v | |
| Flow rate (ml/min) | 38 | ml/min |
| DAD detection | 220 | nm |
| Loop | 1000 | μL |

Example 148 (trans Enantiomer 2) was obtained as first eluted diasteroisomer (8 mg)

Rt.=11.3 min, de >99.9%; LC-MS (ESI): m/z (M+1): 651.3 (Method 2)

Example 149 (cis Enantiomer 2) was obtained as the second eluted diasteroisomer (97 mg)

Rt.=13.8 min, de 99%; LC-MS (ESI): m/z (M+1): 651.3 (Method 2)

### Example 150: propan-2-yl 1-[6-(5-chloro-2-fluorophenyl)-4-({2-[3-(4-methylpiperazin-1-yl)propanamido]pyridin-4-yl}amino)pyridazin-3-yl]azetidine-2-carboxylate

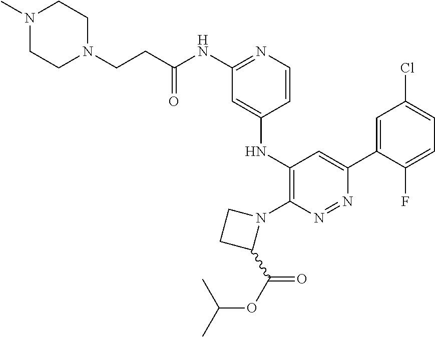 [SMILES: CC(C)OC(=O)C1CCN1c1nnc(-c2cc(Cl)ccc2F)cc1Nc1ccnc(NC(=O)CCN2CCN(C)CC2)c1]

Example 150 was prepared following the procedure used for the synthesis of Example 115, starting from Intermediate 285 (180 mg, 0.49 mmol) and Intermediate 2 (205 mg, 0.61 mmol), to afford title compound (21 mg, 0.03 mmol, 7% yield) as racemic mixture. LC-MS (ESI): m/z (M+1): 611.3 (Method 2)

¹H NMR (500 MHz, Chloroform-d) δ ppm 10.94 (br. s, 1H), 8.11-8.20 (m, 2H), 8.04 (d, J=1.9 Hz, 1H), 7.78 (d, J=1.2 Hz, 1H), 7.31-7.37 (m, 1H), 7.27 (s, 1H), 7.10 (dd, J=10.6, 8.8 Hz, 1H), 6.75 (dd, J=5.6, 2.1 Hz, 1H), 5.14 (spt, J=6.3 Hz, 1H), 4.93 (dd, J=9.5, 7.5 Hz, 1H), 4.56 (q, J=8.5 Hz, 1H), 4.11 (td, J=8.7, 4.5 Hz, 1H), 2.75-2.82 (m, 2H), 2.63-2.75 (m, 1H), 2.52-2.59 (m, 3H), 2.44-3.08 (m, 8H), 2.40 (s, 3H), 1.13-1.34 (m, 6H).

### Example 151: ammonium 1-[6-(5-chloro-2-fluorophenyl)-4-({2-[3-(4-methylpiperazin-1-yl)propanamido]pyridin-4-yl}amino)pyridazin-3-yl]azetidine-2-carboxylate

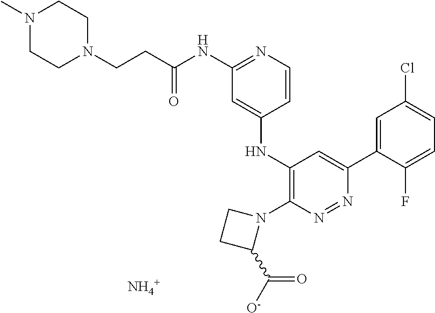 [SMILES: CN1CCN(CCC(=O)Nc2cc(Nc3cc(-c4cc(Cl)ccc4F)nnc3N3CCC3C(=O)[O-])ccn2)CC1 ; [NH4+]]

To a stirred solution of Example 150 (8 mg, 0.01 mmol) in THE (0.150 mL) and MeOH (50 μL) at RT, a solution of lithium hydroxide hydrate (0.6 mg, 0.01 mmol) in H₂O (30 μL) was added and the resulting reaction mixture was heated to 40° C. for 90 min. The mixture was concentrated under vacuum. The residue was purified by reverse flash chromatography on Biotage C18 cartridge (from H₂O+0.1% NH₄OH to 65% MeCN) to afford title compound (4 mg, 0.007 mmol, 52% yield).

LC-MS (ESI): m/z (M+1): 569.2 (Method 4)

¹H NMR (400 MHz, Methanol-d₄) δ ppm 8.42 (s, 1H) 8.08 (d, J=5.7 Hz, 1H) 7.95-8.00 (m, 1H) 7.88-7.94 (m, 3H) 7.71 (d, J=1.5 Hz, 1H) 7.43-7.49 (m, 1H) 7.25 (dd, J=10.5, 8.9 Hz, 1H) 7.16 (d, J=0.9 Hz, 4H) 6.91 (dd, J=5.7, 2.2 Hz, 1H) 4.79-4.83 (m, 2H) 4.39-4.50 (m, 1H) 4.08 (td, J=9.0, 5.0 Hz, 1H) 3.15 (d, J=1.5 Hz, 4H) 2.77-2.91 (m, 5H) 2.76 (s, 3H) 2.67-2.74 (m, 1H) 2.61-2.66 (m, 2H) 2.47-2.57 (m, 1H).

### Example 152: N-(4-{[6-(5-chloro-2-fluorophenyl)-3-({[3-(hydroxymethyl)-2-oxooxolan-3-yl]methyl}(methyl)amino)pyridazin-4-yl]amino}pyridin-2-yl)-3-(4-methylpiperazin-1-yl)propanamide

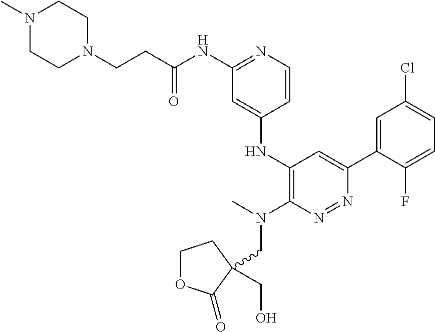 [SMILES: CN1CCN(CCC(=O)Nc2cc(Nc3cc(-c4cc(Cl)ccc4F)nnc3N(C)CC3(CO)CCOC3=O)ccn2)CC1]

Example 152 was prepared following the procedure used for the synthesis of Example 22 starting from Intermediate 292 (38 mg, 0.05 mmol) to afford title compound (25 mg, 0.04 mmol, 78% yield). LC-MS (ESI): m/z (M+1): 627.2 (Method 4)

¹H NMR (500 MHz, Chloroform-d) δ ppm 11.10 (br. s, 1H), 8.21 (d, J=5.6 Hz, 1H), 8.12-8.17 (m, 2H), 7.92 (s, 1H), 7.86 (d, J=1.1 Hz, 1H), 7.37 (ddd, J=8.7, 4.2, 2.9 Hz, 1H), 7.13 (dd, J=10.4, 8.9 Hz, 1H), 6.92 (dd, J=5.6, 2.1 Hz, 1H), 4.30-4.44 (m, 2H), 3.75-3.88 (m, 2H), 3.56-3.69 (m, 2H), 2.96 (s, 3H), 2.76 (br. t, J=5.9 Hz, 2H), 2.55 (br. t, J=5.9 Hz, 2H), 2.39-2.48 (m, 1H), 2.39-2.86 (m, 8H), 2.38 (s, 3H), 2.26 (ddd, J=13.4, 7.7, 6.0 Hz, 1H).

### Example 153: N-(4-{[6-(5-chloro-2-fluorophenyl)-3-[(2-hydroxyethyl)sulfanyl]pyridazin-4-yl]amino}pyridin-2-yl)-3-(piperazin-1-yl)propanamide

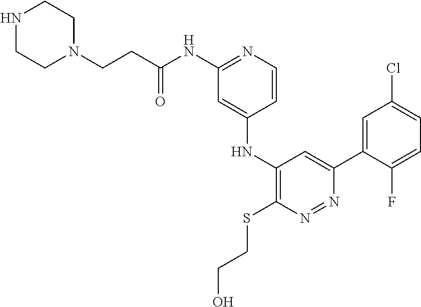 [SMILES: O=C(CCN1CCNCC1)Nc1cc(Nc2cc(-c3cc(Cl)ccc3F)nnc2SCCO)ccn1]

Example 153 was prepared following the procedure used for the synthesis of Example 23 starting from Intermediate 293 (140 mg, 0.19 mmol) to afford title compound (59 mg, 0.11 mmol, 59% yield). LC-MS (ESI): m/z (M+1): 532.3 (Method 2)

¹H NMR (400 MHz, Chloroform-d) δ ppm 11.46 (s, 1H), 8.23 (d, J=5.7 Hz, 1H), 8.14 (dd, J=6.7, 2.6 Hz, 1H), 8.06 (d, J=1.9 Hz, 1H), 7.73 (s, 1H), 7.33-7.44 (m, 1H), 7.13 (dd, J=10.4, 9.0 Hz, 1H), 6.90 (dd, J=5.5, 2.0 Hz, 1H), 6.50 (s, 1H), 4.07 (t, J=5.5 Hz, 2H), 3.66 (t, J=5.5 Hz, 2H), 3.02-3.12 (m, 4H), 2.71-2.78 (m, 2H), 2.61 (br. s, 4H), 2.53-2.58 (m, 2H).

### Example 154: N-(4-{[6-(5-chloro-2-fluorophenyl)-3-{[(3-hydroxycyclobutyl)methyl]sulfanyl}pyridazin-4-yl]amino}pyridin-2-yl)-3-(4-methylpiperazin-1-yl)propanamide

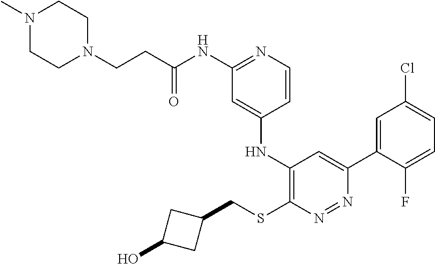

Example 154 was prepared following the procedure used for the synthesis of Example 23 starting from cis N-[4-({3-[({3-[(tert-butyldimethylsilyl)oxy]cyclobutyl}methyl)sulfanyl]-6-(5-chloro-2-fluorophenyl)pyridazin-4-yl}amino)pyridin-2-yl]-3-(4-methylpiperazin-1-yl)propanamide (Intermediate 298, 91 mg, 0.13 mmol) to afford title compound (50 mg, 0.08 mmol, 66% yield). LC-MS (ESI): m/z (M+1): 586.2 (Method 2)

¹H NMR (500 MHz, Methanol-d₄) δ ppm 8.15 (d, J=6.0 Hz, 1H), 8.07 (d, J=1.5 Hz, 1H), 7.94 (dd, J=6.4, 2.7 Hz, 1H), 7.68 (d, J=1.0 Hz, 1H), 7.46-7.56 (m, 1H), 7.27 (dd, J=10.4, 8.9 Hz, 1H), 6.97 (dd, J=5.7, 2.1 Hz, 1H), 3.99-4.10 (m, 1H), 3.52 (d, J=7.1 Hz, 2H), 2.74-2.80 (m, 2H), 2.61 (t, J=6.8 Hz, 2H), 2.46-2.53 (m, 2H), 2.33-3.05 (m, 8H), 2.30 (s, 3H), 2.20-2.29 (m, 1H), 1.64-1.76 (m, 2H).

### Example 155: Cis N-(4-{[6-(5-chloro-2-fluorophenyl)-3-[(2-hydroxyethyl)sulfanyl]pyridazin-4-yl]amino}pyridin-2-yl)-3-(4-methyl-1,4-diazepan-1-yl)cyclobutane-1-carboxamide

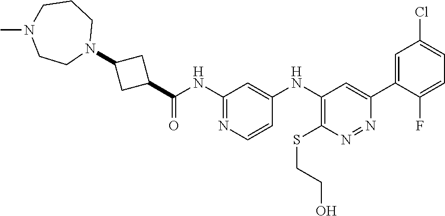

To a solution of Intermediate 301 (30 mg, 0.06 mmol) in Methanol (1.2 mL) was added acetic acid (0.01 mL, 0.18 mmol) and the mixture stirred for 5 minutes at RT. Sodium cyanoborohydride (5 mg, 0.07 mmol) was added and the reaction stirred for 2 hrs. The mixture was concentrated under reduced pressure, loaded onto a SCX cartridge (2 g) and eluted with 1 N NH₃ in MeOH. The organic phase was concentrated under reduced pressure and the crude material was purified by flash chromatography on Biotage NH cartridge (from DCM to 100% MeOH), then by preparative HPLC in basic conditions to afford title compound (4 mg, 0.01 mmol, 11% yield). Only the major isomer cis was isolated. LC-MS (ESI): m/z (M+1): 586.2 (Method 2)

¹H NMR (500 MHz, Methanol-d₄) δ ppm 8.15 (d, J=5.8 Hz, 1H), 8.09 (s, 1H), 7.96 (dd, J=6.5, 2.7 Hz, 1H), 7.71 (d, J=1.1 Hz, 1H), 7.45-7.59 (m, 1H), 7.28 (dd, J=10.4, 8.9 Hz, 1H), 6.97 (dd, J=5.8, 2.2 Hz, 1H), 3.91 (t, J=6.3 Hz, 2H), 3.59 (t, J=6.3 Hz, 2H), 2.87-3.08 (m, 2H), 2.70-2.81 (m, 4H), 2.57-2.67 (m, 4H), 2.38 (s, 3H), 2.32-2.40 (m, 2H), 2.09-2.23 (m, 2H), 1.84 (quin, J=5.8 Hz, 2H).

### Example 156 (trans) and Example 157 (cis): N-(4-{[6-(5-chloro-2-fluorophenyl)-3-[(2-hydroxyethyl)sulfanyl]pyridazin-4-yl]amino}pyridin-2-yl)-3-[4-(propan-2-yl)piperazin-1-yl]cyclobutane-1-carboxamide

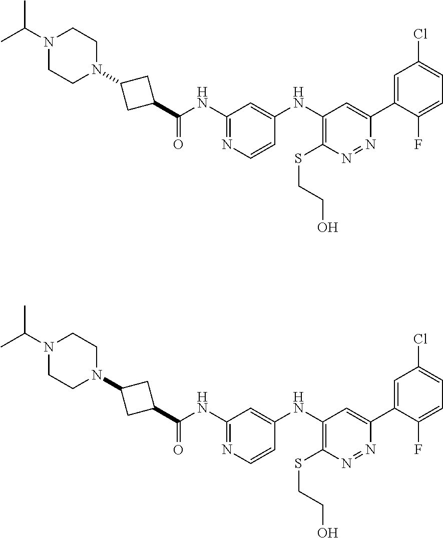

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-[4-(propan-2-yl)piperazin-1-yl]cyclobutane-1-carboxamide (89 mg, 0.10 mmol, 72% yield) was prepared following the procedure used for the synthesis of Example 155, starting from Intermediate 301 (100 mg, 0.20 mmol) and 1-isopropylpiperazine (34 mg, 0.27 mmol). It was separated into the single diasteroisomers by preparative chiral HPLC.

### Conditions:

| Column | Chiralpak IC (25 × 3.0 cm), 5μ | |
|---|---|---|
| Mobile phase | n-Hexane/(Ethanol + 0.1% | |
| | isopropylamine) 60/40% v/v | |
| Flow rate (ml/min) | 40 | ml/min |
| DAD detection | 220 | nm |
| Loop | 1800 | μL |

Example 156 (trans) was obtained as first eluted diasteroisomer (5 mg)

Rt.=9 min, de >99.9%; LC-MS (ESI): m/z (M+1): 600.2 (Method 2)

¹H NMR (500 MHz, DMSO-d₆) δ ppm 10.27 (br. s, 1H), 8.87 (br. s, 1H), 8.04-8.17 (m, 2H), 8.00 (dd, J=6.5, 2.7 Hz, 1H), 7.66 (br. s, 1H), 7.53-7.63 (m, 1H), 7.42 (dd, J=10.4, 9.0 Hz, 1H), 6.91 (br. d, J=4.1 Hz, 1H), 5.09 (br. t, J=5.2 Hz, 1H), 3.69-3.80 (m, 2H), 3.49 (br. t, J=6.4 Hz, 2H), 3.16 (td, J=9.1, 4.5 Hz, 1H), 2.82 (quin, J=7.2 Hz, 1H), 2.58 (dt, J=13.0, 6.5 Hz, 1H), 2.17-2.23 (m, 2H), 2.09-2.47 (m, 8H), 1.94-2.07 (m, 2H), 0.95 (d, J=6.4 Hz, 6H).

Example 157 (cis) was obtained as the second eluted diasteroisomer (54 mg)

Rt.=11.2 min, de >99.9%; LC-MS (ESI): m/z (M+1): 600.2 (Method 2)

¹H NMR (500 MHz, DMSO-d₆) δ ppm 10.35 (s, 1H), 8.89 (br. s, 1H), 8.10 (d, J=5.6 Hz, 1H), 8.07 (br. s, 1H), 8.00 (dd, J=6.5, 2.7 Hz, 1H), 7.66 (s, 1H), 7.56-7.63 (m, 1H), 7.42 (dd, J=10.4, 8.9 Hz, 1H), 6.92 (dd, J=5.6, 2.1 Hz, 1H), 5.09 (br. s, 1H), 3.67-3.82 (m, 2H), 3.50 (t, J=6.4 Hz, 2H), 2.97 (quin, J=8.7 Hz, 1H), 2.54-2.66 (m, 2H), 2.13-2.20 (m, 2H), 2.09-2.46 (m, 8H), 1.92-2.02 (m, 2H), 0.94 (d, J=6.6 Hz, 6H).

### Example 158 (trans) and Example 159 (cis): N-(4-{[6-(5-chloro-2-fluorophenyl)-3-[(2-hydroxyethyl)sulfanyl]pyridazin-4-yl]amino}pyridin-2-yl)-3-(4-ethylpiperazin-1-yl)cyclobutane-1-carboxamide

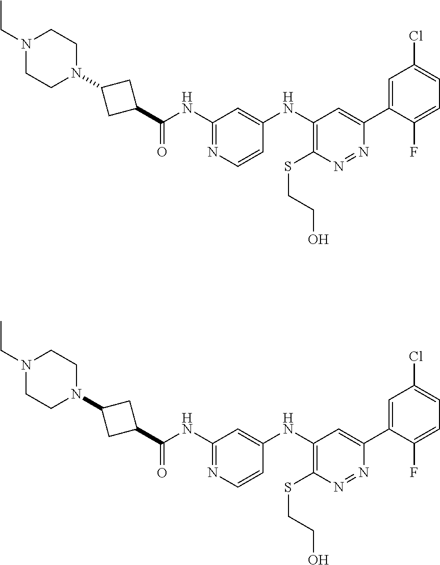

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-(4-ethylpiperazin-1-yl)cyclobutane-1-carboxamide (104 mg, 0.18 mmol, 87% yield) was prepared following the procedure used for the synthesis of Example 155, starting from Intermediate 301 (100 mg, 0.20 mmol) and 1-ethylpiperazine (28 mg, 0.25 mmol). It was separated into the single diasteroisomers by preparative chiral HPLC.

### Conditions:

| Column | Chiralpak IC (25 × 3.0 cm), 5μ | |
|---|---|---|
| Mobile phase | n-Hexane/(Ethanol + 0.1% | |
| | isopropylamine) 60/40% v/v | |
| Flow rate (ml/min) | 40 | ml/min |
| DAD detection | 220 | nm |
| Loop | 1600 | μL |

Example 158 (trans) was obtained as first eluted diasteroisomer (5 mg)

Rt.=10.1 min, de >99.9%; LC-MS (ESI): m/z (M+1): 586.2 (Method 2)

¹H NMR (500 MHz, DMSO-d₆) δ ppm 10.28 (s, 1H), 8.88 (br. s, 1H), 8.06-8.14 (m, 2H), 8.01 (dd, J=6.6, 2.7 Hz, 1H), 7.68 (s, 1H), 7.58-7.64 (m, 1H), 7.42 (dd, J=10.4, 8.9 Hz, 1H), 6.92 (dd, J=5.6, 2.0 Hz, 1H), 5.09 (t, J=5.4 Hz, 1H), 3.74 (q, J=6.3 Hz, 2H), 3.50 (t, J=6.5 Hz, 2H), 3.11-3.24 (m, 1H), 2.83 (quin, J=7.2 Hz, 1H), 2.29 (q, J=7.2 Hz, 2H), 2.16-2.23 (m, 2H), 2.08-2.47 (m, 8H), 1.99-2.09 (m, 2H), 0.97 (t, J=7.2 Hz, 3H).

Example 159 (cis) was obtained as the second eluted diasteroisomer (64 mg)

Rt.=11.7 min, de 99%; LC-MS (ESI): m/z (M+1): 586.2 (Method 2)

¹H NMR (500 MHz, DMSO-d₆) δ ppm 10.34 (s, 1H), 8.88 (br. s, 1H), 8.10 (d, J=5.6 Hz, 1H), 8.07 (s, 1H), 8.00 (dd, J=6.6, 2.7 Hz, 1H), 7.66 (s, 1H), 7.57-7.63 (m, 1H), 7.42 (dd, J=10.4, 8.9 Hz, 1H), 6.92 (dd, J=5.6, 2.1 Hz, 1H), 5.09 (br. s, 1H), 3.74 (br. d, J=2.3 Hz, 2H), 3.50 (t, J=6.5 Hz, 2H), 2.97 (quin, J=8.7 Hz, 1H), 2.54-2.67 (m, 1H), 2.28 (q, J=7.2 Hz, 2H), 2.13-2.20 (m, 2H), 2.03-2.47 (m, 8H), 1.93-2.02 (m, 2H), 0.97 (t, J=7.1 Hz, 3H).

### Example 160: Cis N-(4-{[6-(5-chloro-2-fluorophenyl)-3-[(2-hydroxyethyl)sulfanyl]pyridazin-4-yl]amino}pyridin-2-yl)-3-(4-cyclopropylpiperazin-1-yl)cyclobutane-1-carboxamide

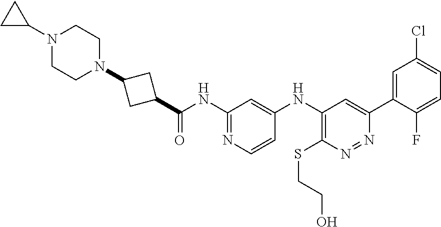

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-(4-cyclopropylpiperazin-1-yl)cyclobutane-1-carboxamide (70 mg, 0.12 mmol, 95% yield) was prepared following the procedure used for the synthesis of Example 155, starting from Intermediate 301 (60 mg, 0.12 mmol) and 1-cyclopropylpiperazine (28 mg, 0.25 mmol). It was separated into the single diasteroisomers by preparative chiral HPLC.

### Conditions:

| Column | Chiralpak AD-H (25 × 3.0 cm), 5μ | |
|---|---|---|
| Mobile phase | n-Hexane/(Ethanol + 0.1% | |
| | isopropylamine) 65/35% v/v | |
| Flow rate (ml/min) | 39 | ml/min |
| DAD detection | 220 | nm |
| Loop | 1000 | μL |

Only Example 160 (cis) was obtained as second eluted diasteroisomer (43 mg)

Rt.=12.1 min, de 99%; LC-MS (ESI): m/z (M+1): 598.2 (Method 2)

¹H NMR (500 MHz, Chloroform-d) δ ppm 9.01 (s, 1H), 8.21 (d, J=5.6 Hz, 1H), 8.15 (dd, J=6.7, 2.7 Hz, 1H), 8.08 (d, J=1.9 Hz, 1H), 7.73 (d, J=1.1 Hz, 1H), 7.40 (ddd, J=8.7, 4.2, 2.7 Hz, 1H), 7.13 (dd, J=10.5, 8.9 Hz, 1H), 6.93 (dd, J=5.6, 2.1 Hz, 1H), 6.54 (s, 1H), 4.07 (t, J=5.6 Hz, 2H), 3.60-3.72 (m, 2H), 3.42 (br. s, 1H), 2.92 (quin, J=8.3 Hz, 1H), 2.77-2.84 (m, 1H), 2.42-2.50 (m, 2H), 2.31-2.84 (m, 8H), 2.21-2.30 (m, 2H), 1.64-1.72 (m, 1H), 0.37-0.53 (m, 4H).

### Example 161 (trans) and Example 162 (cis): N-(4-{[6-(5-chloro-2-fluorophenyl)-3-[(2-hydroxyethyl)sulfanyl]pyridazin-4-yl]amino}pyridin-2-yl)-3-[4-fluoro-4-(hydroxymethyl)piperidin-1-yl]cyclobutane-1-carboxamide

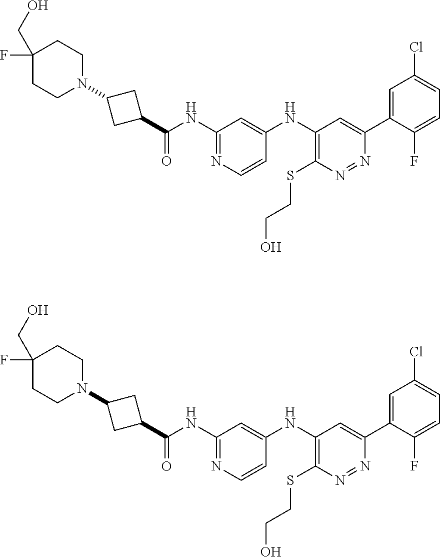

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-[4-fluoro-4-(hydroxymethyl)piperidin-1-yl]cyclobutane-1-carboxamide (90 mg, 0.15 mmol, 76% yield) was prepared following the procedure used for the synthesis of Example 155, starting from Intermediate 301 (95 mg, 0.19 mmol) and 4-fluoro-4-piperidinemethanol hydrochloride (169 mg, 1 mmol). It was separated into the single diasteroisomers by preparative chiral HPLC.

### Conditions:

| Column | Chiralpak IC (25 × 3.0 cm), 5μ | |
|---|---|---|
| Mobile phase | n-Hexane/(Ethanol + 0.1% | |
| | isopropylamine) 65/35% v/v | |
| Flow rate (ml/min) | 30 | ml/min |
| DAD detection | 220 | nm |
| Loop | 1350 | μL |

Example 161 (trans) was obtained as first eluted diasteroisomer (5 mg)

Rt.=11.1 min, de >99.9%; LC-MS (ESI): m/z (M+1): 605.4 (Method 2)

¹H NMR (500 MHz, Chloroform-d) δ ppm 8.21 (d, J=5.6 Hz, 1H), 8.12-8.18 (m, 2H), 7.88 (s, 1H), 7.76 (d, J=1.0 Hz, 1H), 7.38-7.45 (m, 1H), 7.14 (dd, J=10.6, 8.8 Hz, 1H), 6.95 (dd, J=5.6, 2.2 Hz, 1H), 6.52 (s, 1H), 4.08 (br. t, J=5.4 Hz, 2H), 3.67 (t, J=5.6 Hz, 2H), 3.61 (d, J=20.5 Hz, 2H), 3.16-3.37 (m, 1H), 3.03-3.14 (m, 2H), 2.70 (br. d, J=11.1 Hz, 2H), 2.50 (ddd, J=13.2, 7.5, 3.0 Hz, 2H), 2.23-2.35 (m, 2H), 2.11-2.20 (m, 2H), 1.97 (br. t, J=11.8 Hz, 2H), 1.59-1.81 (m, 2H).

Example 162 (cis) was obtained as second eluted diasteroisomer (74 mg)

Rt.=13.3 min, de >99.9%; LC-MS (ESI): m/z (M+1): 605.4 (Method 2)

¹H NMR (500 MHz, Chloroform-d) δ ppm 9.89 (br. s, 1H), 8.17 (d, J=5.6 Hz, 1H), 8.13 (dd, J=6.7, 2.7 Hz, 1H), 8.08 (d, J=1.9 Hz, 1H), 7.72 (d, J=1.0 Hz, 1H), 7.36-7.42 (m, 1H), 7.13 (dd, J=10.6, 8.8 Hz, 1H), 6.91 (dd, J=5.6, 2.1 Hz, 1H), 6.58 (s, 1H), 4.07 (t, J=5.6 Hz, 2H), 3.62-3.70 (m, 4H), 3.44-3.63 (m, 1H), 2.97 (quin, J=7.9 Hz, 1H), 2.81-2.89 (m, 3H), 2.48-2.58 (m, 2H), 2.15-2.30 (m, 4H), 1.80-2.04 (m, 4H).

### Example 163 (trans) and Example 164 (cis): N-(4-{[6-(5-chloro-2-fluorophenyl)-3-[(2-hydroxyethyl)sulfanyl]pyridazin-4-yl]amino}pyridin-2-yl)-3-(4-methoxypiperidin-1-yl)cyclobutane-1-carboxamide

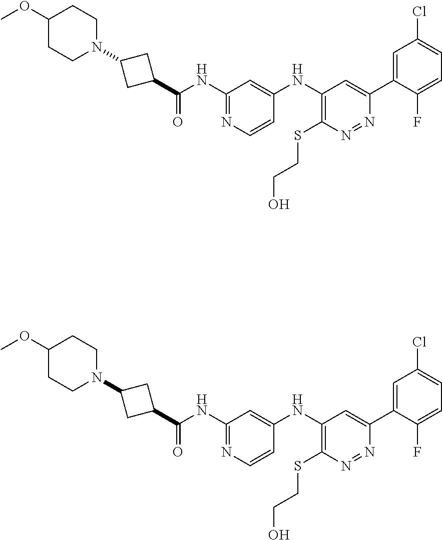

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-(4-methoxypiperidin-1-yl)cyclobutane-1-carboxamide (90 mg, 0.15 mmol, 78% yield) was prepared following the procedure used for the synthesis of Example 155, starting from Intermediate 301 (96 mg, 0.20 mmol) and 4-methoxypiperidine (59 mg, 0.51 mmol). It was separated into the single diasteroisomers by preparative chiral HPLC.

### Conditions:

| Column | Chiralpak IC (25 × 3.0 cm), 5μ | |
|---|---|---|
| Mobile phase | n-Hexane/(Ethanol + 0.1% | |
| | isopropylamine) 72/28% v/v | |
| Flow rate (ml/min) | 38 | ml/min |
| DAD detection | 220 | nm |
| Loop | 950 | μL |

Example 163 (trans) was obtained as first eluted diasteroisomer (5 mg)

Rt.=19.8 min, de >99.9%; LC-MS (ESI): m/z (M+1): 587.4 (Method 2)

¹H NMR (500 MHz, Chloroform-d) δ ppm 8.20 (d, J=5.6 Hz, 1H), 8.11-8.18 (m, 2H), 8.00 (br. s, 1H), 7.75 (d, J=1.1 Hz, 1H), 7.37-7.44 (m, 1H), 7.10-7.17 (m, 1H), 6.94 (dd, J=5.6, 2.2 Hz, 1H), 6.52 (br. s, 1H), 4.02-4.13 (m, 2H), 3.63-3.71 (m, 2H), 3.35 (s, 3H), 3.17-3.30 (m, 1H), 2.96-3.12 (m, 2H), 2.61-2.76 (m, 2H), 2.41-2.54 (m, 2H), 2.24-2.36 (m, 2H), 1.70-2.07 (m, 6H).

Example 164 (cis) was obtained as second eluted diasteroisomer (67 mg)

Rt.=22 min, de >99.9%; LC-MS (ESI): m/z (M+1): 587.4 (Method 2)

¹H NMR (500 MHz, Chloroform-d) δ ppm 9.08-9.29 (m, 1H), 8.20 (d, J=5.6 Hz, 1H), 8.14 (dd, J=6.7, 2.7 Hz, 1H), 8.08 (d, J=1.9 Hz, 1H), 7.72 (d, J=1.1 Hz, 1H), 7.39 (ddd, J=8.8, 4.2, 2.8 Hz, 1H), 7.13 (dd, J=10.5, 8.9 Hz, 1H), 6.91 (dd, J=5.6, 2.2 Hz, 1H), 6.58 (s, 1H), 4.07 (t, J=5.6 Hz, 2H), 3.62-3.69 (m, 2H), 3.36 (s, 3H), 3.29 (dt, J=7.4, 3.9 Hz, 1H), 2.91 (quin, J=8.3 Hz, 1H), 2.77 (quin, J=7.0 Hz, 1H), 2.62-2.73 (m, 2H), 2.42-2.52 (m, 2H), 2.20-2.30 (m, 2H), 2.06-2.19 (m, 2H), 1.91-2.02 (m, 2H), 1.63-1.79 (m, 2H).

### Example 165 (trans) and Example 166 (cis): ethyl 1-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}piperidine-4-carboxylate

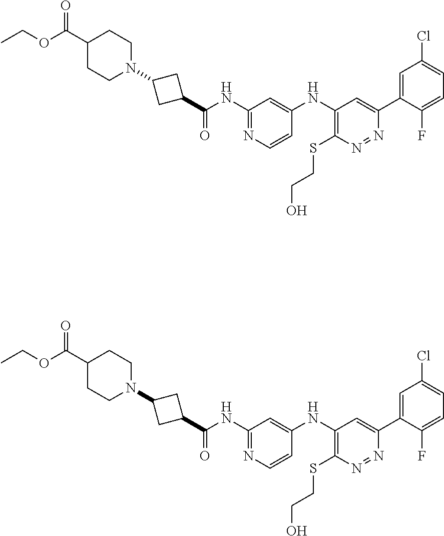

Diasteroisomeric mixture of cis and trans ethyl 1-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}piperidine-4-carboxylate (85 mg, 0.15 mmol, 82% yield) was prepared following the procedure used for the synthesis of Example 155, starting from Intermediate 301 (95 mg, 0.19 mmol) and 4-piperidinecarboxylic acid ethyl ester (72 mg, 0.46 mmol). It was separated into the single diasteroisomers by preparative chiral HPLC.

### Conditions:

| Column | Chiralpak AD-H (25 × 2.0 cm), 5μ | |
|---|---|---|
| Mobile phase | n-Hexane/(Ethanol/Methanol 1/1 + 0.1% | |
| | isopropylamine) 30/70% v/v | |
| Flow rate (ml/min) | 17 | ml/min |
| DAD detection | 220 | nm |
| Loop | 1000 | μL |

Example 165 (trans) was obtained as first eluted diasteroisomer (4 mg)

Rt.=7 min, de >99.9%; LC-MS (ESI): m/z (M+1): 629.4 (Method 2)

¹H NMR (600 MHz, Chloroform-d) δ ppm 8.21 (d, J=5.6 Hz, 1H), 8.16 (dd, J=6.7, 2.7 Hz, 1H), 8.14 (d, J=1.6 Hz, 1H), 7.90 (s, 1H), 7.75 (d, J=0.8 Hz, 1H), 7.38-7.43 (m, 1H), 7.14 (dd, J=10.5, 8.8 Hz, 1H), 6.94 (dd, J=5.6, 2.1 Hz, 1H), 6.51 (s, 1H), 4.14 (q, J=7.1 Hz, 2H), 4.08 (t, J=5.6 Hz, 2H), 3.64-3.69 (m, 2H), 3.23 (br. s, 1H), 3.03-3.09 (m, 1H), 2.98-3.04 (m, 1H), 2.84 (br. d, J=9.4 Hz, 2H), 2.47 (ddd, J=13.3, 7.5, 3.0 Hz, 2H), 2.22-2.37 (m, 3H), 1.93 (br. d, J=12.0 Hz, 2H), 1.80-1.88 (m, 2H), 1.72-1.79 (m, 2H), 1.26 (t, J=7.1 Hz, 3H).

Example 166 (cis) was obtained as second eluted diasteroisomer (60 mg)

Rt.=10.3 min, de >99.9%; LC-MS (ESI): m/z (M+1): 629.4 (Method 2)

¹H NMR (400 MHz, Chloroform-d) δ ppm 9.15 (s, 1H), 8.21 (d, J=5.6 Hz, 1H), 8.14 (dd, J=6.6, 2.7 Hz, 1H), 8.07 (d, J=1.8 Hz, 1H), 7.73 (s, 1H), 7.40 (ddd, J=8.7, 4.0, 3.0 Hz, 1H), 7.14 (dd, J=10.5, 8.9 Hz, 1H), 6.92 (dd, J=5.6, 2.0 Hz, 1H), 6.53 (s, 1H), 4.11-4.20 (m, 2H), 4.07 (t, J=5.5 Hz, 2H), 3.66 (t, J=5.5 Hz, 2H), 2.84-2.99 (m, 3H), 2.76 (quin, J=7.0 Hz, 1H), 2.38-2.54 (m, 2H), 2.18-2.37 (m, 3H), 1.82-2.02 (m, 6H), 1.22-1.34 (m, 3H).

### Example 167: Cis 1-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}piperidine-4-carboxylic acid

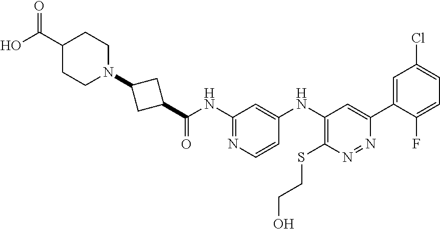

Example 167 was prepared following the procedure used for the synthesis of Example 99, starting from Example 166 (15 mg, 0.02 mmol) to afford title compound (12 mg, 0.028 mmol, 76% yield). LC-MS (ESI): m/z (M+1): 601.3 (Method 4)

¹H NMR (500 MHz, DMSO-d₆) δ ppm 12.10 (br. s, 1H), 10.33 (br. s, 1H), 8.03-8.13 (m, 2H), 8.01 (dd, J=6.5, 2.7 Hz, 1H), 7.66 (br. s, 1H), 7.63 (s, 1H), 7.58-7.62 (m, 1H), 7.43 (dd, J=10.4, 8.9 Hz, 1H), 6.92 (br. d, J=4.3 Hz, 1H), 5.09 (br. s, 1H), 3.69-3.77 (m, 2H), 3.50 (br. t, J=6.4 Hz, 2H), 2.90-3.02 (m, 1H), 2.68 (br. d, J=10.4 Hz, 2H), 2.52-2.60 (m, 2H), 2.07-2.22 (m, 3H), 1.92-2.04 (m, 2H), 1.70-1.80 (m, 4H), 1.42-1.55 (m, 1H).

### Example 168 (trans) and Example 169 (cis): N-(4-{[6-(5-chloro-2-fluorophenyl)-3-[(2-hydroxyethyl)sulfanyl]pyridazin-4-yl]amino}pyridin-2-yl)-3-(4-methylpiperidin-1-yl)cyclobutane-1-carboxamide

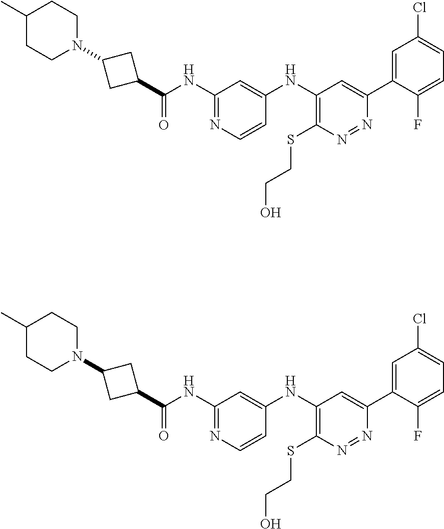

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-(4-methylpiperidin-1-yl)cyclobutane-1-carboxamide (0.16 mmol, 95 mg, quantitative yield) was prepared following the procedure used for the synthesis of Example 155, starting from Intermediate 301 (80 mg, 0.16 mmol) and 4-methylpiperidine (41 mg, 0.41 mmol). It was separated into the single diasteroisomers by preparative HPLC in basic conditions to afford:

Example 168 (trans) was obtained as first eluted diasteroisomer (2 mg)

LC-MS (ESI): Rt.=0.97 min. m/z (M+1): 571.3 (Method 2)

¹H NMR (600 MHz, Chloroform-d) δ ppm 8.21 (d, J=5.6 Hz, 1H), 8.16 (dd, J=6.7, 2.7 Hz, 1H), 8.14 (d, J=1.6 Hz, 1H), 7.86 (s, 1H), 7.75 (s, 1H), 7.40 (ddd, J=8.7, 4.1, 2.8 Hz, 1H), 7.14 (dd, J=10.5, 8.8 Hz, 1H), 6.94 (dd, J=5.6, 2.0 Hz, 1H), 6.51 (s, 1H), 4.08 (br. t, J=5.4 Hz, 2H), 3.67 (t, J=5.6 Hz, 2H), 3.25 (br. s, 1H), 3.02-3.08 (m, 1H), 2.99 (quin, J=7.7 Hz, 1H), 2.86 (br. d, J=11.4 Hz, 2H), 2.44-2.50 (m, 2H), 2.25-2.33 (m, 2H), 1.72 (br. t, J=11.7 Hz, 2H), 1.66 (br. d, J=11.5 Hz, 2H), 1.33-1.43 (m, 1H), 1.23 (qd, J=12.4, 3.6 Hz, 2H), 0.94 (d, J=6.6 Hz, 3H).

Example 169 (cis) was obtained as second eluted diasteroisomer (43 mg)

LC-MS (ESI): Rt.=1.01 min, m/z (M+1): 571.3 (Method 2)

¹H NMR (600 MHz, Chloroform-d) δ ppm 9.25 (s, 1H), 8.20 (d, J=5.6 Hz, 1H), 8.14 (dd, J=6.6, 2.6 Hz, 1H), 8.08 (d, J=1.8 Hz, 1H), 7.72 (s, 1H), 7.39 (ddd, J=8.7, 4.0, 2.8 Hz, 1H), 7.13 (dd, J=10.4, 8.9 Hz, 1H), 6.92 (dd, J=5.6, 2.0 Hz, 1H), 6.56 (s, 1H), 4.07 (t, J=5.5 Hz, 2H), 3.66 (t, J=5.5 Hz, 2H), 3.51 (br. s, 1H), 2.92-2.97 (m, 2H), 2.88-2.93 (m, 1H), 2.73 (quin, J=7.0 Hz, 1H), 2.42-2.51 (m, 2H), 2.20-2.29 (m, 2H), 1.79 (br. t, J=11.0 Hz, 2H), 1.64-1.72 (m, 2H), 1.32-1.45 (m, 3H), 0.96 (d, J=5.6 Hz, 3H).

### Example 170 (trans) and Example 171 (cis): N-(4-{[6-(5-chloro-2-fluorophenyl)-3-[(2-hydroxyethyl)sulfanyl]pyridazin-4-yl]amino}pyridin-2-yl)-3-[4,4-difluoro-3-(hydroxymethyl)piperidin-1-yl]cyclobutane-1-carboxamide

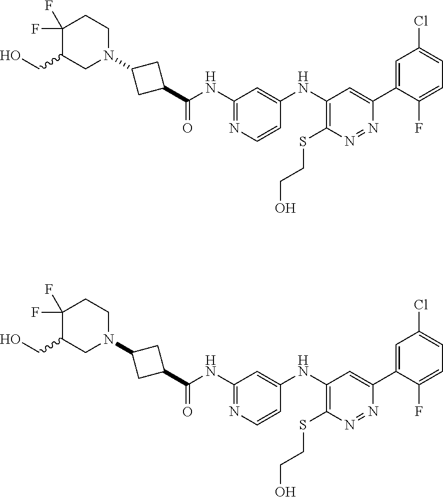

Diasteroisomeric mixture of racemic cis and trans N-(4-{[6-(5-chloro-2-fluorophenyl)-3-[(2-hydroxyethyl)sulfanyl]pyridazin-4-yl]amino}pyridin-2-yl)-3-[4,4-difluoro-3-(hydroxymethyl)piperidin-1-yl]cyclobutane-1-carboxamide (90 mg, 0.14 mmol, 88% yield) was prepared following the procedure used for the synthesis of Example 155, starting from Intermediate 301 (80 mg, 0.16 mmol) and (4,4-difluoro-3-piperidyl)methanol (62 mg, 0.41 mmol). It was purified by preparative HPLC in basic condition to afford:

Example 170 (trans) was obtained as first eluted racemic diasteroisomer (9 mg)

LC-MS (ESI): Rt.=0.81 min, m/z (M+1): 623.2 (Method 2)

¹H NMR (400 MHz, Chloroform-d) δ ppm 8.21 (d, J=5.7 Hz, 1H), 8.16 (dd, J=6.7, 2.6 Hz, 1H), 8.12 (d, J=1.4 Hz, 1H), 7.93 (s, 1H), 7.75 (s, 1H), 7.36-7.44 (m, 1H), 7.14 (dd, J=10.5, 8.9 Hz, 1H), 6.95 (dd, J=5.6, 2.0 Hz, 1H), 6.51 (s, 1H), 4.08 (t, J=5.5 Hz, 2H), 3.95-4.02 (m, 1H), 3.86 (br. dd, J=11.3, 4.0 Hz, 1H), 3.67 (t, J=5.5 Hz, 2H), 3.24 (br. s, 1H), 3.01-3.15 (m, 2H), 2.37-2.74 (m, 6H), 1.96-2.37 (m, 6H).

Example 171 (cis) was obtained as second eluted racemic diasteroisomer (43 mg)

LC-MS (ESI): Rt.=0.88 min, m/z (M+1): 623.2 (Method 2)

¹H NMR (400 MHz, Chloroform-d) δ ppm 8.91 (br. s, 1H), 8.19 (d, J=5.6 Hz, 1H), 8.14 (dd, J=6.6, 2.7 Hz, 1H), 8.08 (d, J=1.6 Hz, 1H), 7.73 (s, 1H), 7.37-7.43 (m, 1H), 7.14 (dd, J=10.4, 9.0 Hz, 1H), 6.93 (dd, J=5.6, 2.0 Hz, 1H), 6.59 (s, 1H), 4.07 (t, J=5.5 Hz, 2H), 3.97 (dd, J=11.2, 4.3 Hz, 1H), 3.85 (br. dd, J=11.2, 5.8 Hz, 1H), 3.66 (t, J=5.5 Hz, 2H), 2.93 (quin, J=8.1 Hz, 1H), 2.83 (quin, J=7.0 Hz, 1H), 2.75 (br. d, J=8.0 Hz, 1H), 2.44-2.63 (m, 5H), 2.01-2.40 (m, 5H).

### Example 172: Cis N-(4-{[6-(5-chloro-2-fluorophenyl)-3-[(2-hydroxyethyl)sulfanyl]pyridazin-4-yl]amino}pyridin-2-yl)-3-[3-(2-fluoroethyl)-4-methylpiperazin-1-yl]cyclobutane-1-carboxamide

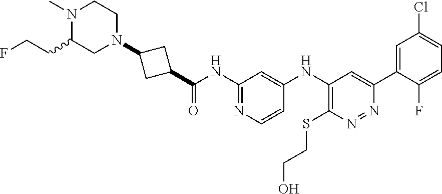

Diasteroisomeric mixture of racemic cis and trans N-(4-{[6-(5-chloro-2-fluorophenyl)-3-[(2-hydroxyethyl)sulfanyl]pyridazin-4-yl]amino}pyridin-2-yl)-3-[3-(2-fluoroethyl)-4-methylpiperazin-1-yl]cyclobutane-1-carboxamide (70 mg, 0.12 mmol, 95% yield) was prepared following the procedure used for the synthesis of Example 155, starting from Intermediate 301 (70 mg, 0.14 mmol) and 2-(2-fluoroethyl)-1-methyl-piperazine dihydrochloride (79 mg, 0.36 mmol). It was separated into the single diasteroisomers by preparative HPLC in basic conditions.

Only Example 172 (cis) was obtained as second eluted racemic diasteroisomer (5 mg). LC-MS (ESI): Rt.=0.86 min, m/z (M+1): 618.2 (Method 2)

¹H NMR (500 MHz, Chloroform-d) δ ppm 8.91 (s, 1H), 8.20 (d, J=5.6 Hz, 1H), 8.15 (dd, J=6.6, 2.7 Hz, 1H), 8.08 (d, J=1.6 Hz, 1H), 7.74 (s, 1H), 7.37-7.43 (m, 1H), 7.14 (dd, J=10.4, 8.9 Hz, 1H), 6.93 (dd, J=5.6, 2.1 Hz, 1H), 6.53 (br. s, 1H), 4.44-4.67 (m, 2H), 4.07 (t, J=5.5 Hz, 2H), 3.66 (t, J=5.6 Hz, 2H), 2.92 (quin, J=8.3 Hz, 1H), 2.72-2.85 (m, 4H), 2.44-2.52 (m, 3H), 2.37-2.44 (m, 1H), 2.32 (s, 3H), 2.19-2.28 (m, 2H), 2.13 (br. t, J=10.1 Hz, 1H), 1.97-2.10 (m, 1H), 1.88-1.95 (m, 1H), 1.76-1.89 (m, 1H).

### Example 173 (trans) and Example 174 (cis): N-(4-{[6-(5-chloro-2-fluorophenyl)-3-[(2-hydroxyethyl)sulfanyl]pyridazin-4-yl]amino}pyridin-2-yl)-3-{5-methyl-5,8-diazaspiro[3.5]nonan-8-yl}cyclobutane-1-carboxamide

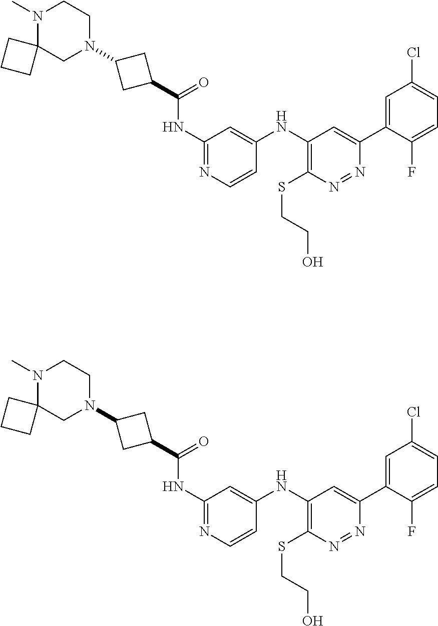

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-{5-methyl-5,8-diazaspiro[3.5]nonan-8-yl}cyclobutane-1-carboxamide (72 mg, 0.12 mmol, 79% yield) was prepared following the procedure used for the synthesis of Example 155, starting from Intermediate 303 (0.14 mmol) and formaldehyde 37% w/w in water (12 μl, 11.2 mmol). It was separated into the single diasteroisomers by preparative chiral HPLC.

### Conditions:

| Column | Chiralpak IC (25 × 3.0 cm), 5μ | |
|---|---|---|
| Mobile phase | n-Hexane/(Ethanol + 0.1% | |
| | isopropylamine) 70/30% v/v | |
| Flow rate (ml/min) | 38 | ml/min |
| DAD detection | 220 | nm |
| Loop | 1150 | μL |

Example 173 (trans) was obtained as first eluted diasteroisomer (3 mg)

Rt.=15.3 min, de >99.9%; LC-MS (ESI): m/z (M+1): 612.2 (Method 2)

¹H NMR (400 MHz, Chloroform-d) δ ppm 8.21 (d, J=5.7 Hz, 1H), 8.12-8.18 (m, 2H), 7.90 (s, 1H), 7.75 (s, 1H), 7.40 (dt, J=8.8, 3.5 Hz, 1H), 7.14 (dd, J=10.4, 9.1 Hz, 1H), 6.94 (dd, J=5.6, 1.9 Hz, 1H), 6.52 (s, 1H), 4.08 (t, J=5.5 Hz, 2H), 3.66 (t, J=5.5 Hz, 2H), 2.98-3.13 (m, 2H), 2.35-2.40 (m, 3H), 1.97-2.91 (m, 14H), 1.66-1.80 (m, 2H).

Example 174 (cis) was obtained as second eluted diasteroisomer (41 mg)

Rt.=17.2 min, de >99.9%; LC-MS (ESI): m/z (M+1): 612.2 (Method 2)

¹H NMR (400 MHz, Chloroform-d) δ ppm 8.70 (s, 1H), 8.20 (d, J=5.5 Hz, 1H), 8.15 (dd, J=6.6, 2.6 Hz, 1H), 8.09 (d, J=1.8 Hz, 1H), 7.74 (s, 1H), 7.35-7.45 (m, 1H), 7.07-7.19 (m, 1H), 6.93 (dd, J=5.5, 2.0 Hz, 1H), 6.52 (s, 1H), 4.07 (t, J=5.6 Hz, 2H), 3.66 (t, J=5.5 Hz, 2H), 2.92 (quin, J=8.4 Hz, 1H), 2.80 (quin, J=7.1 Hz, 1H), 2.52-2.58 (m, 2H), 2.38 (s, 3H), 2.16-2.52 (m, 12H), 1.47-1.88 (m, 2H).

### Example 175: Cis N-(4-{[6-(5-chloro-2-fluorophenyl)-3-[(2-hydroxyethyl)sulfanyl]pyridazin-4-yl]amino}pyridin-2-yl)-3-{6-methyl-3,6-diazabicyclo[3.1.1]heptan-3-yl}cyclobutane-1-carboxamide

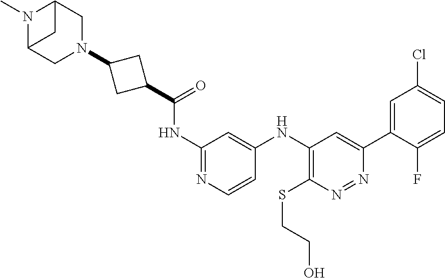

Diasteroisomeric mixture of cis and trans N-(4-{[6-(5-chloro-2-fluorophenyl)-3-[(2-hydroxyethyl)sulfanyl]pyridazin-4-yl]amino}pyridin-2-yl)-3-{6-methyl-3,6-diazabicyclo[3.1.1]heptan-3-yl}cyclobutane-1-carboxamide (43 mg, 0.07 mmol, 43% yield) was prepared following the procedure used for the synthesis of Example 155, starting from Intermediate 305 (0.17 mmol) and formaldehyde 37% w/w in water (14 μl, 17 mmol). It was separated into the enricheddiasteroisomers by preparative chiral HPLC.

### Conditions:

| Column | Chiralcel OD-H (25 × 2.0 cm), 5μ | |
|---|---|---|
| Mobile phase | n-Hexane/(Ethanol + 0.1% | |
| | isopropylamine) 60/40% v/v | |
| Flow rate (ml/min) | 17 | ml/min |
| DAD detection | 220 | nm |
| Loop | 2500 | μL |

Only Example 175 (cis) was obtained (22 mg).

Rt.=5.8 min, de 92% LC-MS (ESI): m/z (M+1): 584.2 (Method 2)

¹H NMR (500 MHz, DMSO-d₆) δ ppm 10.35 (s, 1H), 8.27-9.57 (m, 1H), 8.05-8.13 (m, 2H), 8.00 (dd, J=6.6, 2.7 Hz, 1H), 7.64-7.72 (m, 1H), 7.60 (ddd, J=8.9, 4.1, 2.8 Hz, 1H), 7.35-7.49 (m, 1H), 6.92 (dd, J=5.6, 2.1 Hz, 1H), 4.66-5.45 (m, 1H), 3.73 (t, J=6.5 Hz, 2H), 3.49 (t, J=6.5 Hz, 2H), 3.29-3.36 (m, 2H), 3.14-3.23 (m, 1H), 2.98 (quin, J=8.7 Hz, 1H), 2.81 (d, J=10.8 Hz, 2H), 2.68 (br. d, J=10.7 Hz, 2H), 2.11-2.29 (m, 5H), 1.95 (s, 3H), 1.78 (d, J=7.4 Hz, 1H).

### Example 176: N-(6-{[6-(5-chloro-2-fluorophenyl)-3-[(2-hydroxyethyl)sulfanyl]pyridazin-4-yl]amino}pyrimidin-4-yl)-3-(4-methylpiperazin-1-yl)propanamide

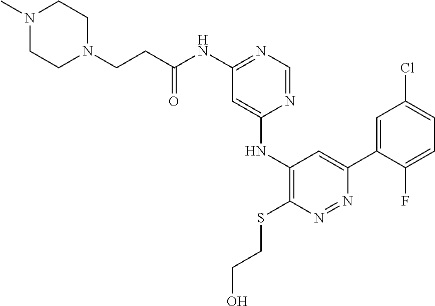 [SMILES: CN1CCN(CCC(=O)Nc2cc(Nc3cc(-c4cc(Cl)ccc4F)nnc3SCCO)ncn2)CC1]

Example 176 was prepared following the procedure used for the synthesis of Example 23 starting from Intermediate 306 (30 mg, 0.04 mmol) to afford title compound (11 mg, 0.02 mmol, 45% yield). LC-MS (ESI): m/z (M+1): 547.2 (Method 2)

¹H NMR (400 MHz, Chloroform-d) δ ppm 11.74 (br. s, 1H), 9.03 (s, 1H), 8.60 (s, 1H), 8.12 (dd, J=6.6, 2.6 Hz, 1H), 7.77 (s, 1H), 7.41 (dt, J=8.6, 3.5 Hz, 1H), 7.15-7.21 (m, 1H), 7.15 (s, 1H), 4.07 (br. s, 2H), 3.65 (t, J=5.5 Hz, 2H), 3.39 (br. s, 1H), 2.74-2.79 (m, 2H), 2.55-2.60 (m, 2H), 2.45-2.95 (m, 8H), 2.39 (s, 3H).

### Example 177: N-(6-{[6-(5-chloro-2-fluorophenyl)-3-[(2-hydroxyethyl)sulfanyl]pyridazin-4-yl]amino}pyrimidin-4-yl)-3-(3,5-dimethylpiperazin-1-yl)propanamide

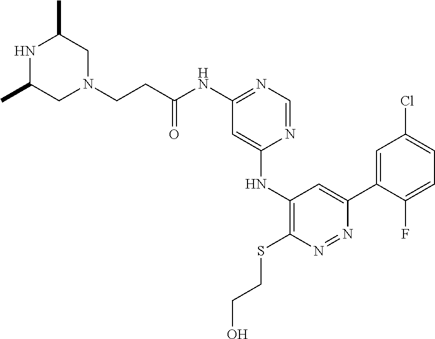 [SMILES: C[C@@H]1CN(CCC(=O)Nc2cc(Nc3cc(-c4cc(Cl)ccc4F)nnc3SCCO)ncn2)C[C@H](C)N1]

Example 177 was prepared following the procedure used for the synthesis of Example 23 starting from Intermediate 309 (80 mg, 0.10 mmol) to afford title compound (34 mg, 0.06 mmol, 59% yield). LC-MS (ESI): m/z (M+1): 561.3 (Method 4)

¹H NMR (500 MHz, Chloroform-d) δ ppm 11.95 (br. s, 1H), 9.03 (d, J=1.4 Hz, 1H), 8.58 (d, J=0.8 Hz, 1H), 8.12 (dd, J=6.6, 2.7 Hz, 1H), 7.77 (d, J=0.8 Hz, 1H), 7.36-7.45 (m, 1H), 7.08-7.21 (m, 2H), 4.07 (t, J=5.6 Hz, 2H), 3.65 (t, J=5.5 Hz, 2H), 3.27-3.57 (m, 1H), 3.11-3.23 (m, 2H), 2.95 (br. d, J=9.5 Hz, 2H), 2.70-2.77 (m, 2H), 2.51-2.62 (m, 2H), 1.81 (br. t, J=10.1 Hz, 2H), 1.13 (br. d, J=6.2 Hz, 6H).

### Example 178: N-(4-{[6-(5-chloro-2-fluorophenyl)-3-[(2-hydroxyethyl)sulfanyl]pyridazin-4-yl]amino}pyridin-2-yl)-3-(3,5-dimethylpiperazin-1-yl)propanamide

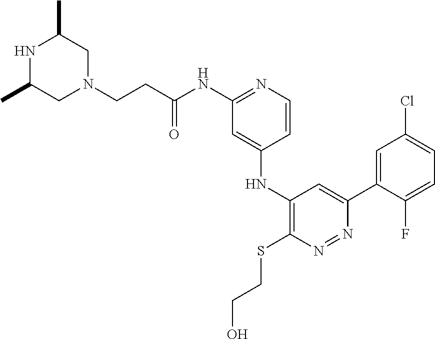 [SMILES: C[C@@H]1CN(CCC(=O)Nc2cc(Nc3cc(-c4cc(Cl)ccc4F)nnc3SCCO)ccn2)C[C@H](C)N1]

Example 178 was prepared following the procedure used for the synthesis of Example 23 starting from Intermediate 311 (160 mg, 0.21 mmol) to afford title compound (93 mg, 0.17 mmol, 80% yield). LC-MS (ESI): m/z (M+1): 560.3 (Method 4).

¹H NMR (400 MHz, Chloroform-d) δ ppm 11.43 (br. s, 1H), 8.22 (d, J=5.6 Hz, 1H), 8.14 (dd, J=6.6, 2.6 Hz, 1H), 8.06 (d, J=1.6 Hz, 1H), 7.72 (s, 1H), 7.39 (dt, J=8.2, 3.6 Hz, 1H), 7.08-7.17 (m, 1H), 6.90 (dd, J=5.6, 1.6 Hz, 1H), 6.51 (s, 1H), 4.07 (t, J=5.5 Hz, 2H), 3.66 (t, J=5.5 Hz, 2H), 3.11-3.25 (m, 2H), 2.97 (br. d, J=10.2 Hz, 2H), 2.70-2.80 (m, 2H), 2.53-2.62 (m, 2H), 1.83 (br. t, J=8.2 Hz, 2H), 1.14 (br. d, J=6.1 Hz, 6H).

### Example 179: N-(4-{[6-(5-chloro-2-fluorophenyl)-3-sulfanylpyridazin-4-yl]amino}pyridin-2-yl)-3-(4-methylpiperazin-1-yl)propanamide

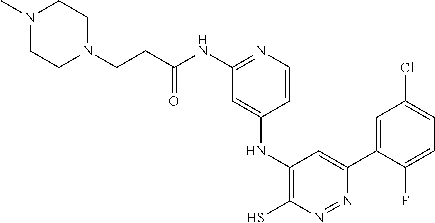 [SMILES: CN1CCN(CCC(=O)Nc2cc(Nc3cc(-c4cc(Cl)ccc4F)nnc3S)ccn2)CC1]

Tetrabutylammonium fluoride 1M in THE (0.71 mL, 0.71 mmol) was added to a solution of Intermediate 314 (390 mg, 0.65 mmol) in THE (8 mL). The mixture was stirred at RT for 5 hrs, then volatiles were removed under vacuum to afford a residue that was triturated with water. The solid was collected by filtration, washed with water, and dried under vacuum. The solid was again triturated with Et₂O, then with MeOH, filtered and dried under vacuum to afford title compound (61 mg, 0.12 mmol, 18% yield).

LC-MS (ESI): m/z (M+1): 502.3 (Method 4)

¹H NMR (500 MHz, DMSO-d₆) δ ppm 15.01 (br. s, 1H), 10.75 (s, 1H), 9.07 (s, 1H), 8.25 (d, J=5.6 Hz, 1H), 8.15 (s, 1H), 7.84 (dd, J=6.5, 2.7 Hz, 1H), 7.55-7.70 (m, 1H), 7.42 (s, 1H), 7.43 (dd, J=10.3, 8.9 Hz, 1H), 7.19 (dd, J=5.6, 2.1 Hz, 1H), 2.59-2.66 (m, 2H), 2.52-2.56 (m, 2H), 2.22-2.49 (m, 8H), 2.16 (s, 3H).

### Example 180: N-(4-{[6-(5-chloro-2-fluorophenyl)-3-[(2-hydroxyethyl)sulfanyl]pyridazin-4-yl]amino}pyridin-2-yl)-3-[(4-methylpiperazin-1-yl)methyl]bicyclo[11.1.1]pentane-1-carboxamide

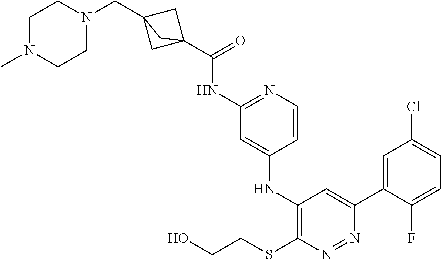 [SMILES: CN1CCN(CC23CC(C(=O)Nc4cc(Nc5cc(-c6cc(Cl)ccc6F)nnc5SCCO)ccn4)(C2)C3)CC1]

Example 180 was prepared following the procedure used for the synthesis of Example 23 starting from Intermediate 318 (133 mg, 0.19 mmol) to afford title compound (60 mg, 0.10 mmol, 54% yield). LC-MS (ESI): m/z (M+1): 598.4 (Method 2)

¹H NMR (600 MHz, Chloroform-d) δ ppm 8.21 (d, J=5.6 Hz, 1H), 8.15 (dd, J=6.7, 2.7 Hz, 1H), 8.08 (d, J=2.1 Hz, 1H), 7.88 (s, 1H), 7.73 (d, J=1.0 Hz, 1H), 7.40 (ddd, J=8.7, 4.3, 2.8 Hz, 1H), 7.13 (dd, J=10.5, 8.7 Hz, 1H), 6.94 (dd, J=5.6, 2.1 Hz, 1H), 6.52 (s, 1H), 4.07 (t, J=5.6 Hz, 2H), 3.66 (t, J=5.6 Hz, 2H), 3.31 (br. s, 1H), 2.51 (s, 2H), 2.46 (br. s, 8H), 2.30 (s, 3H), 2.10 (s, 6H).

### Example 181: Cis N-(6-{[6-(5-chloro-2-fluorophenyl)-3-[(2-hydroxyethyl)sulfanyl]pyridazin-4-yl]amino}pyrimidin-4-yl)-3-(4-cyclopropylpiperazin-1-yl)cyclobutane-1-carboxamide

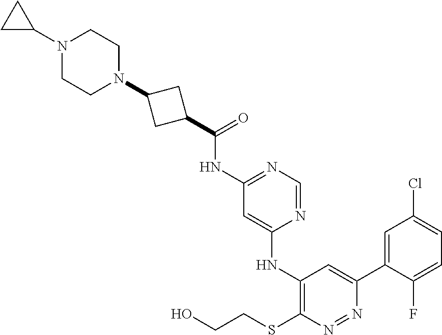

Example 181 was prepared following the procedure used for the synthesis of Example 23 starting from Intermediate 321 (110 mg, 0.15 mmol) to afford title compound (50 mg, 0.08 mmol, 54% yield). LC-MS (ESI): m/z (M+1): 599.2 (Method 2)

¹H NMR (400 MHz, Chloroform-d) δ ppm 10.16 (s, 1H), 9.05 (s, 1H), 8.60 (s, 1H), 8.13 (dd, J=6.6, 2.6 Hz, 1H), 7.80 (s, 1H), 7.38-7.45 (m, 1H), 7.12-7.20 (m, 2H), 4.07 (q, J=5.2 Hz, 2H), 3.65 (t, J=5.5 Hz, 2H), 3.43 (br. t, J=5.4 Hz, 1H), 3.00 (br. t, J=7.8 Hz, 1H), 2.74-2.92 (m, 1H), 2.49-2.61 (m, 2H), 2.35-2.91 (m, 8H), 2.18-2.33 (m, 2H), 1.71 (br. s, 1H), 0.38-0.55 (m, 4H)

### Example 182: N-(6-{[6-(5-chloro-2-fluorophenyl)-3-[(2-hydroxyethyl)sulfanyl]pyridazin-4-yl]amino}pyrimidin-4-yl)-3-[(4-methylpiperazin-1-yl)methyl]bicyclo[1.1.1]pentane-1-carboxamide

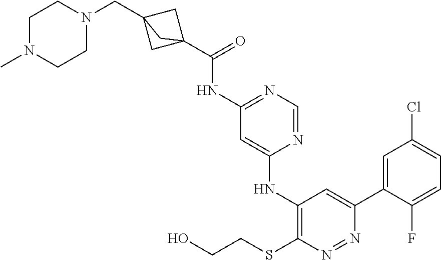 [SMILES: CN1CCN(CC23CC(C(=O)Nc4cc(Nc5cc(-c6cc(Cl)ccc6F)nnc5SCCO)ncn4)(C2)C3)CC1]

Example 182 was prepared following the procedure used for the synthesis of Example 23 starting from Intermediate 323 (37 mg, 0.05 mmol) to afford title compound (13 mg, 0.02 mmol, 42% yield). LC-MS (ESI): m/z (M+1): 599.2 (Method 4)

¹H NMR (500 MHz, Chloroform-d) δ ppm 9.04 (d, J=1.4 Hz, 1H), 8.60 (d, J=0.8 Hz, 1H), 8.13 (dd, J=6.6, 2.7 Hz, 1H), 7.89 (s, 1H), 7.79 (d, J=0.8 Hz, 1H), 7.37-7.46 (m, 1H), 7.22 (s, 1H), 7.17 (dd, J=10.4, 8.9 Hz, 1H), 4.06 (t, J=5.5 Hz, 2H), 3.64 (t, J=5.5 Hz, 2H), 2.52-2.56 (m, 2H), 2.42-2.83 (m, 8H), 2.33-2.45 (m, 3H), 2.12 (s, 6H).

### Example 183: N-(4-{[6-(5-chloro-2-fluorophenyl)-3-[(2-hydroxyethyl)sulfanyl]pyridazin-4-yl]amino}pyridin-2-yl)-3-[(4-cyclopropylpiperazin-1-yl)methyl]bicyclo[1.1.1]pentane-1-carboxamide

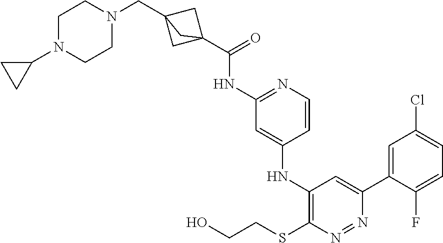 [SMILES: O=C(Nc1cc(Nc2cc(-c3cc(Cl)ccc3F)nnc2SCCO)ccn1)C12CC(CN3CCN(C4CC4)CC3)(C1)C2]

Example 183 was prepared following the procedure used for the synthesis of Example 23 starting from Intermediate 326 (125 mg, 0.17 mmol) to afford title compound (62 mg, 0.10 mmol, 59% yield). LC-MS (ESI): m/z (M+1): 624.2 (Method 4)

¹H NMR (400 MHz, Chloroform-d) δ ppm 8.21 (d, J=5.7 Hz, 1H), 8.15 (dd, J=6.6, 2.6 Hz, 1H), 8.08 (d, J=2.0 Hz, 1H), 7.89 (br. s, 1H), 7.73 (s, 1H), 7.37-7.44 (m, 1H), 7.13 (dd, J=10.4, 8.9 Hz, 1H), 6.94 (dd, J=5.5, 2.0 Hz, 1H), 6.52 (s, 1H), 4.07 (br. s, 2H), 3.66 (t, J=5.5 Hz, 2H), 3.17-3.38 (m, 1H), 2.35-2.92 (m, 10H), 2.11 (s, 6H), 1.60-1.72 (m, 1H), 0.27-0.60 (m, 4H).

### Example 184: propan-2-yl 1-[6-(5-chloro-2-fluorophenyl)-4-({2-[3-(3,5-dimethylpiperazin-1-yl)propanamido]pyridin-4-yl}amino)pyridazin-3-yl]azetidine-2-carboxylate

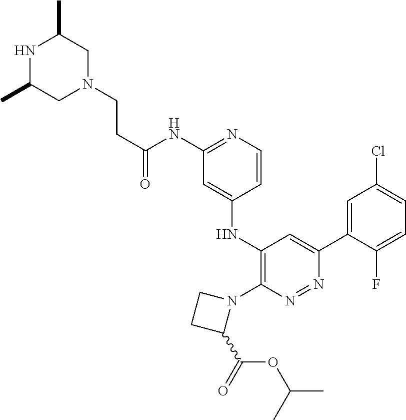 [SMILES: CC(C)OC(=O)C1CCN1c1nnc(-c2cc(Cl)ccc2F)cc1Nc1ccnc(NC(=O)CCN2C[C@@H](C)N[C@@H](C)C2)c1]

Example 184 was prepared following the procedure used for the synthesis of Example 23 starting from Intermediate 327 (30 mg, 0.04 mmol) to afford title compound (11 mg, 0.02 mmol, 42% yield). LC-MS (ESI): m/z (M+1): 625.3 (Method 4)

¹H NMR (400 MHz, Chloroform-d) δ ppm 11.27 (br. s, 1H), 8.10-8.19 (m, 2H), 8.05 (d, J=1.8 Hz, 1H), 7.77 (d, J=1.1 Hz, 1H), 7.33 (ddd, J=8.6, 4.1, 2.9 Hz, 1H), 7.24 (s, 1H), 7.10 (dd, J=10.5, 9.0 Hz, 1H), 6.74 (dd, J=5.6, 2.1 Hz, 1H), 5.13 (quin, J=6.2 Hz, 1H), 4.94 (dd, J=9.5, 7.6 Hz, 1H), 4.54 (q, J=8.3 Hz, 1H), 4.11 (td, J=8.7, 4.5 Hz, 1H), 3.08-3.22 (m, 2H), 2.95 (br. d, J=11.0 Hz, 2H), 2.64-2.77 (m, 3H), 2.50-2.62 (m, 3H), 1.76 (br. t, J=10.5 Hz, 2H), 1.15-1.32 (m, 6H), 1.10 (d, J=6.4 Hz, 6H).

### Example 185 (trans), example 186 (cis Enantiomer 1), and example 187 (cis Enantiomer 2): N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)cyclobutane-1-carboxamide

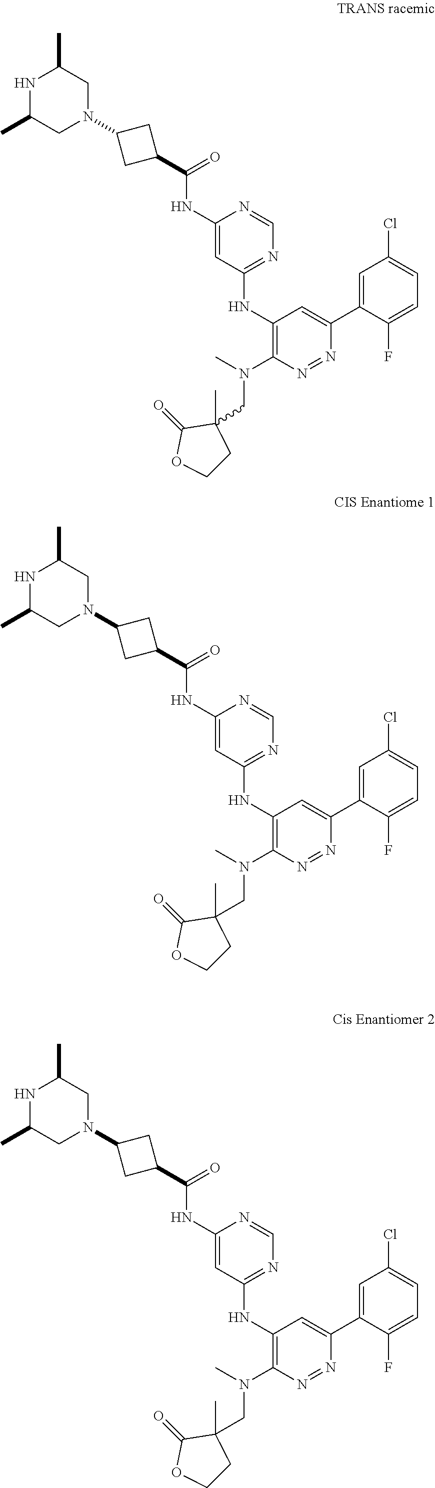

Diasteroisomeric mixture of racemic cis and trans N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)cyclobutane-1-carboxamide (110 mg, 0.17 mmol, 95% yield) was prepared following the procedure used for the synthesis of Example 23, starting from Intermediate 330 (133 mg, 0.18 mmol). It was separated into the diasteroisomers by preparative chiral HPLC.

### Conditions:

| Column | Chiralpak IC (25 × 3.0 cm), 5μ | |
|---|---|---|
| Mobile phase | n-Hexane/(Ethanol + 0.1% | |
| | isopropylamine) 70/30% v/v | |
| Flow rate (ml/min) | 40 | ml/min |
| DAD detection | 220 | nm |
| Loop | 900 | μL |

Example 185 (trans) was obtained as racemic mixture collecting first and second eluted diasteroisomers (7 mg)

Rt.=19.2, 21.8 min, de >99.9%; LC-MS (ESI): m/z (M+1): 652.3 (Method 4)

¹H NMR (500 MHz, Chloroform-d) δ ppm 9.12 (d, J=1.5 Hz, 1H), 8.59 (s, 1H), 8.44 (s, 1H), 8.14 (dd, J=6.7, 2.7 Hz, 1H), 7.93 (s, 1H), 7.89 (br. s, 1H), 7.34-7.44 (m, 1H), 7.16 (dd, J=10.4, 8.9 Hz, 1H), 4.25-4.40 (m, 2H), 3.86 (d, J=14.3 Hz, 1H), 3.72 (d, J=14.3 Hz, 1H), 3.03-3.15 (m, 2H), 2.98 (br. s, 2H), 2.90 (s, 3H), 2.80 (br. d, J=10.6 Hz, 2H), 2.48 (ddd, J=11.4, 7.8, 3.2 Hz, 2H), 2.31 (br. s, 2H), 2.15-2.26 (m, 1H), 1.98-2.09 (m, 1H), 1.52 (br. s, 2H), 1.25 (s, 3H), 1.03-1.19 (m, 6H).

Example 186 (cis Enantiomer 1) was obtained as third eluted diasteroisomer (35 mg)

Rt.=24.8 min, de >99.9%, ee >99.9%; LC-MS (ESI): m/z (M+1): 652.3 (Method 4)

¹H NMR (500 MHz, Chloroform-d) δ ppm 9.71 (br. s, 1H), 9.09 (d, J=1.5 Hz, 1H), 8.59 (d, J=0.7 Hz, 1H), 8.29 (s, 1H), 8.14 (dd, J=6.7, 2.7 Hz, 1H), 7.89 (d, J=0.7 Hz, 1H), 7.34-7.43 (m, 1H), 7.16 (dd, J=10.3, 8.9 Hz, 1H), 4.21-4.41 (m, 2H), 3.78-3.88 (m, 1H), 3.68-3.77 (m, 1H), 3.06-3.22 (m, 2H), 2.98 (quin, J=7.9 Hz, 1H), 2.84-2.93 (m, 5H), 2.79 (quin, J=6.3 Hz, 1H), 2.44-2.56 (m, 2H), 2.19-2.29 (m, 3H), 1.95-2.06 (m, 1H), 1.45-1.54 (m, 2H), 1.25 (s, 3H), 1.10 (br. d, J=6.0 Hz, 6H).

Example 187 (cis Enantiomer 2) was obtained as fourth eluted diasteroisomer (36 mg)

Rt.=27.9 min, de >99.9%, ee 96.5%; LC-MS (ESI): m/z (M+1): 652.3 (Method 4)

### Example 188: Cis Enantiomer 1 N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)cyclopentane-1-carboxamide

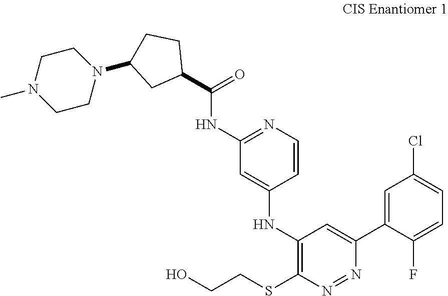

Example 188 was prepared following the procedure used for the synthesis of Example 23 starting from cis Enantiomer 1 N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)cyclopentane-1-carboxamide (Intermediate 333, 120 mg, 0.17 mmol) to afford title compound (61 mg, 0.10 mmol, 61% yield). LC-MS (ESI): m/z (M+1): 586.2 (Method 4)

¹H NMR (500 MHz, Chloroform-d) δ ppm 10.03 (br. s, 1H), 8.19 (d, J=5.6 Hz, 1H), 8.14 (dd, J=6.6, 2.7 Hz, 1H), 8.06 (d, J=1.9 Hz, 1H), 7.72 (s, 1H), 7.35-7.43 (m, 1H), 7.13 (dd, J=10.5, 8.9 Hz, 1H), 6.91 (dd, J=5.6, 2.1 Hz, 1H), 6.52 (s, 1H), 4.07 (t, J=5.5 Hz, 2H), 3.65 (t, J=5.5 Hz, 2H), 3.39 (br. s, 1H), 2.87-2.97 (m, 1H), 2.71-2.77 (m, 1H), 2.66 (br. s, 8H), 2.38 (br. s, 3H), 2.09-2.18 (m, 1H), 2.04-2.09 (m, 2H), 1.94-2.04 (m, 2H), 1.69-1.77 (m, 1H).

### Example 189: Trans Enantiomer 1 N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)cyclopentane-1-carboxamide

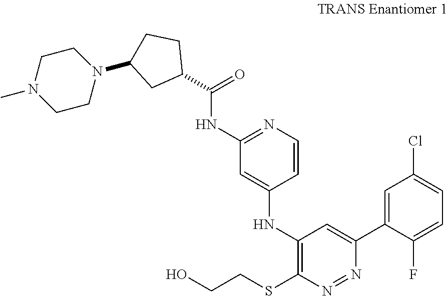

Example 189 was prepared following the procedure used for the synthesis of Example 23 starting from trans Enantiomer 1 N-(4-{[3-({2-[(tert-butyldimethylsilyl)oxy]ethyl}sulfanyl)-6-(5-chloro-2-fluorophenyl)pyridazin-4-yl]amino}pyridin-2-yl)-3-(4-methylpiperazin-1-yl)cyclopentane-1-carboxamide (Intermediate 334, 100 mg, 0.14 mmol) to afford title compound (59 mg, 0.10 mmol, 70% yield). LC-MS (ESI): m/z (M+1): 586.2 (Method 4)

¹H NMR (500 MHz, Chloroform-d) δ ppm 8.21 (d, J=5.6 Hz, 1H), 8.15 (dd, J=6.7, 2.7 Hz, 1H), 8.09 (d, J=1.8 Hz, 1H), 7.91 (s, 1H), 7.74 (s, 1H), 7.40 (ddd, J=8.7, 4.2, 2.8 Hz, 1H), 7.13 (dd, J=10.4, 8.9 Hz, 1H), 6.94 (dd, J=5.6, 2.1 Hz, 1H), 6.51 (s, 1H), 4.07 (t, J=5.6 Hz, 2H), 3.66 (t, J=5.6 Hz, 2H), 3.29 (br. s, 1H), 2.90-2.99 (m, 1H), 2.81 (br. s, 1H), 2.36-3.02 (m, 8H), 2.32 (s, 3H), 2.21-2.35 (m, 1H), 2.05-2.19 (m, 2H), 1.86-1.97 (m, 2H), 1.49-1.71 (m, 1H).

### Example 190: N-(4-{[6-(5-chloro-2-fluorophenyl)-3-{[(5-methyl-2-oxo-2H-1,3-dioxol-4-yl)methyl]sulfanyl}pyridazin-4-yl]amino}pyridin-2-yl)-3-(4-methylpiperazin-1-yl)propanamide

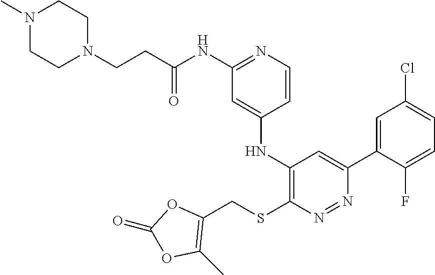 [SMILES: Cc1oc(=O)oc1CSc1nnc(-c2cc(Cl)ccc2F)cc1Nc1ccnc(NC(=O)CCN2CCN(C)CC2)c1]

To a suspension of K₂CO₃ (24 mg, 0.18 mmol) and Example 179 (80 mg, 0.16 mmol) in DMF (2 mL), 4-(chloromethyl)-5-methyl-1,3-dioxol-2-one (26 mg, 0.180 mmol) was added and the mixture was stirred at RT for 30 min. H₂O and DCM were added, the product was extracted with DCM (2×), organic phase was dried over Na₂SO₄, filtered and concentrated under reduced pressure. The crude material was purified by flash chromatography on Biotage silica cartridge (from DCM to 20% MeOH), then further purified by reverse flash chromatography on Biotage C18 cartridge (from H₂O+0.1% NH₄OH to 100% MeCN then 100% MeOH) to afford title compound (9 mg, 0.015 mmol, 9% yield). LC-MS (ESI): m/z (M+1): 614.2 (Method 4)

¹H NMR (400 MHz, Chloroform-d) δ ppm 10.61 (s, 1H), 8.99 (s, 1H), 8.11 (d, J=5.7 Hz, 1H), 8.03 (s, 1H), 7.98 (dd, J=6.6, 2.6 Hz, 1H), 7.69 (s, 1H), 7.54-7.66 (m, 1H), 7.43 (dd, J=10.4, 9.1 Hz, 1H), 6.91 (dd, J=5.5, 1.8 Hz, 1H), 4.60 (s, 2H), 2.56-2.63 (m, 2H), 2.46-2.53 (m, 2H), 2.27-2.54 (m, 8H), 2.21 (s, 3H), 2.14 (s, 3H).

### Example 191: N-(4-{[6-(5-chloro-2-fluorophenyl)-3-[(2-hydroxyethyl)sulfanyl]pyridazin-4-yl]amino}pyridin-2-yl)-3-[(3,5-dimethylpiperazin-1-yl)methyl]bicyclo[1.1.1]pentane-1-carboxamide

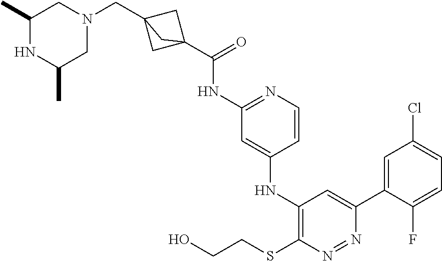 [SMILES: C[C@@H]1CN(CC23CC(C(=O)Nc4cc(Nc5cc(-c6cc(Cl)ccc6F)nnc5SCCO)ccn4)(C2)C3)C[C@H](C)N1]

Example 191 was prepared following the procedure used for the synthesis of Example 23 starting from Intermediate 337 (155 mg, 0.19 mmol) to afford title compound (42 mg, 0.07 mmol, 37% yield). LC-MS (ESI): m/z (M+1): 612.2 (Method 4)

¹H NMR (500 MHz, DMSO-d₆) δ ppm 10.01 (br. s, 1H), 8.90 (br. s, 1H), 8.12 (br. d, J=5.4 Hz, 1H), 7.99 (br. d, J=3.4 Hz, 2H), 7.53-7.79 (m, 2H), 7.42 (t, J=9.6 Hz, 1H), 6.82-7.04 (m, 1H), 5.08 (t, J=5.4 Hz, 1H), 3.73 (q, J=6.2 Hz, 2H), 3.49 (br. t, J=5.1 Hz, 2H), 2.65-2.80 (m, 4H), 2.31 (s, 2H), 1.99 (s, 6H), 1.70-2.02 (m, 1H), 1.46 (t, J=10.2 Hz, 2H), 0.84-0.96 (m, 6H).

### Example 192: N-(4-{[6-(5-chloro-2-fluorophenyl)-3-[(3-methyl-2-oxooxolan-3-yl)sulfanyl]pyridazin-4-yl]amino}pyridin-2-yl)-3-(4-methylpiperazin-1-yl)propanamide

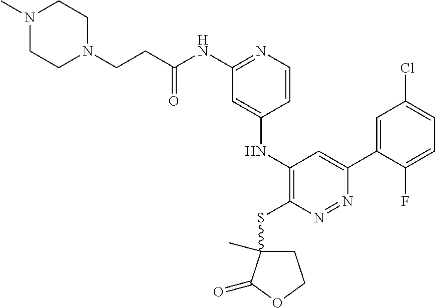 [SMILES: CN1CCN(CCC(=O)Nc2cc(Nc3cc(-c4cc(Cl)ccc4F)nnc3SC3(C)CCOC3=O)ccn2)CC1]

Example 192 was prepared following the procedure used for the synthesis of Example 190 starting from Example 179 (20 mg, 0.04 mmol) and 3-bromodihydro-3-methylfuran-2(3H)-one (28 mg, 0.16 mmol) at 60° C. to afford title compound (3 mg, 0.005 mmol, 12% yield). LC-MS (ESI): m/z (M+1): 600.3 (Method 4)

¹H NMR (500 MHz, Chloroform-d) δ ppm 10.82 (br. s, 1H), 8.14-8.21 (m, 3H), 8.12 (dd, J=6.6, 2.7 Hz, 1H), 7.85 (s, 1H), 7.57 (ddd, J=8.8, 4.0, 3.0 Hz, 1H), 7.34 (dd, J=10.7, 8.9 Hz, 1H), 7.05 (dd, J=5.6, 2.0 Hz, 1H), 4.50-4.60 (m, 1H), 4.44 (td, J=8.4, 6.4 Hz, 1H), 2.98 (ddd, J=13.4, 8.5, 6.4 Hz, 1H), 2.72 (t, J=6.2 Hz, 2H), 2.56-2.61 (m, 1H), 2.52-2.57 (m, 2H), 2.29-2.67 (m, 8H), 2.22 (s, 3H), 1.77 (s, 3H).

### Example 193: N-(4-{[6-(5-chloro-2-fluorophenyl)-3-[(2-hydroxyethyl)sulfanyl]pyridazin-4-yl]amino}pyridin-2-yl)-2-(3,5-dimethylpiperazin-1-yl)acetamide

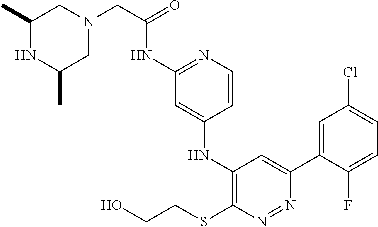 [SMILES: C[C@@H]1CN(CC(=O)Nc2cc(Nc3cc(-c4cc(Cl)ccc4F)nnc3SCCO)ccn2)C[C@H](C)N1]

Example 193 was prepared following the procedure used for the synthesis of Example 23 starting from Intermediate 339 (0.23 mmol) to afford title compound (40 mg, 0.07 mmol, 31% yield). LC-MS (ESI): m/z (M+1): 546.3 (Method 4)

¹H NMR (400 MHz, DMSO-d₆) δ ppm 9.77 (s, 1H), 8.97 (s, 1H), 8.11 (d, J=5.7 Hz, 1H), 8.03 (s, 1H), 7.99 (dd, J=6.5, 2.7 Hz, 1H), 7.67 (s, 1H), 7.55-7.65 (m, 1H), 7.43 (dd, J=10.4, 8.9 Hz, 1H), 6.95 (dd, J=5.7, 1.8 Hz, 1H), 5.08 (t, J=5.4 Hz, 1H), 3.74 (q, J=6.1 Hz, 2H), 3.44-3.59 (m, 2H), 3.10 (s, 2H), 2.80 (br. t, J=6.7 Hz, 2H), 2.67-2.73 (m, 2H), 1.73 (br. t, J=10.4 Hz, 2H), 0.92 (d, J=6.4 Hz, 6H).

### Example 194: N-(4-{[6-(5-chloro-2-fluorophenyl)-3-{[2-(2-hydroxyethoxy)ethyl]sulfanyl}pyridazin-4-yl]amino}pyridin-2-yl)-3-(4-methylpiperazin-1-yl)propanamide

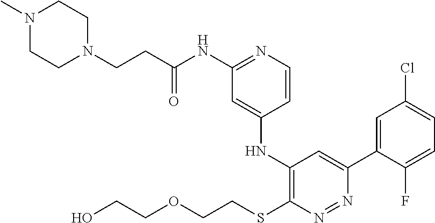 [SMILES: CN1CCN(CCC(=O)Nc2cc(Nc3cc(-c4cc(Cl)ccc4F)nnc3SCCOCCO)ccn2)CC1]

Example 194 was prepared following the procedure used for the synthesis of Example 23 starting from Intermediate 343 (139 mg. 0.20 mol) to afford title compound (40 mg, 0.07 mmol, 31% yield). LC-MS (ESI): m/z (M+1): 590.3 (Method 4)

¹H NMR (500 MHz, DMSO-d₆) δ ppm 10.60 (s, 1H), 8.92 (br. s, 1H), 8.11 (d, J=5.8 Hz, 1H), 8.04 (s, 1H), 7.99 (dd, J=6.6, 2.7 Hz, 1H), 7.65 (br. s, 1H), 7.57-7.62 (m, 1H), 7.42 (dd, J=10.6, 8.9 Hz, 1H), 6.92 (dd, J=5.6, 1.7 Hz, 1H), 4.62 (t, J=5.2 Hz, 1H), 3.72-3.80 (m, 2H), 3.56-3.63 (m, 2H), 3.45-3.55 (m, 4H), 2.56-2.62 (m, 2H), 2.48-2.54 (m, 2H), 2.16-2.49 (m, 8H), 2.14 (s, 3H).

### Example 195: N-(6-{[6-(5-chloro-2-fluorophenyl)-3-{[2-(trimethylsilyl)ethyl]sulfanyl}pyridazin-4-yl]amino}pyrimidin-4-yl)-3-(3,5-dimethylpiperazin-1-yl)propanamide

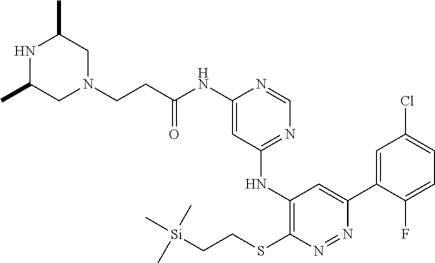 [SMILES: C[C@@H]1CN(CCC(=O)Nc2cc(Nc3cc(-c4cc(Cl)ccc4F)nnc3SCC[Si](C)(C)C)ncn2)C[C@H](C)N1]

Example 195 was prepared following the procedure used for the synthesis of Example 23 starting from Intermediate 344 (100 mg. 0.14 mol) to afford title compound (62 mg, 0.10 mmol, 71% yield). LC-MS (ESI): m/z (M+1): 617.3 (Method 4)

¹H NMR (500 MHz, DMSO-d₆) δ ppm 11.05 (br. s, 1H), 9.39 (br. s, 1H), 8.48 (s, 1H), 8.44 (s, 1H), 7.99 (dd, J=6.6, 2.7 Hz, 1H), 7.95 (br. s, 1H), 7.59-7.66 (m, 1H), 7.46 (dd, J=10.4, 8.9 Hz, 1H), 3.36-3.45 (m, 2H), 2.70-2.81 (m, 4H), 2.53-2.62 (m, 4H), 1.68-2.32 (m, 1H), 1.51 (br. t, J=10.8 Hz, 2H), 1.00-1.09 (m, 2H), 0.92 (d, J=6.2 Hz, 6H), 0.08 (s, 9H).

### Example 196: 3-({[6-(5-chloro-2-fluorophenyl)-4-({2-[3-(4-methylpiperazin-1-yl)propanamido]pyridin-4-yl}amino)pyridazin-3-yl]sulfanyl}methyl)benzoic acid

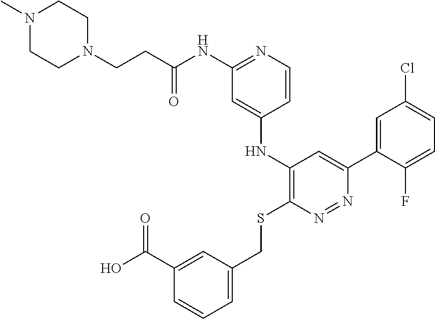 [SMILES: CN1CCN(CCC(=O)Nc2cc(Nc3cc(-c4cc(Cl)ccc4F)nnc3SCc3cccc(C(=O)O)c3)ccn2)CC1]

### Step 1

To a suspension of K₂CO₃ (20 mg 0.140 mmol) and Example 179 (71 mg, 0.14 mmol) in MeCN (3 mL), methyl 3-(bromomethyl)benzoate (25 mg, 0.11 mmol) was added and the mixture was stirred at RT for 1 h. Solids were removed by filtration, volatiles were removed under vacuum. The crude material was purified by flash chromatography on Biotage NH cartridge (from cHex to 100% EtOAc) and then by flash chromatography on Biotage NH (from DCM to 2% of MeOH) to afford methyl 3-({[6-(5-chloro-2-fluorophenyl)-4-({2-[3-(4-methylpiperazin-1-yl)propanamido]pyridin-4-yl}amino)pyridazin-3-yl]sulfanyl}methyl)benzoate (68 mg, 0.104 mmol, 95% yield).

### Step 2

Example 196 was prepared following the procedure used for the synthesis of Example 99, starting from methyl 3-({[6-(5-chloro-2-fluorophenyl)-4-({2-[3-(4-methylpiperazin-1-yl)propanamido]pyridin-4-yl}amino)pyridazin-3-yl]sulfanyl}methyl)benzoate (from Step 1, 30 mg, 0.05 mmol) to afford title compound (7 mg, 0.01 mmol, 24% yield). LC-MS (ESI): m/z (M+1): 636.2 (Method 4)

¹H NMR (400 MHz, DMSO-d₆) δ ppm 11.35-14.30 (m, 1H), 10.59 (s, 1H), 8.91 (s, 1H), 8.10 (d, J=5.7 Hz, 1H), 8.07 (s, 1H), 8.02 (s, 1H), 8.00 (dd, J=6.6, 2.4 Hz, 1H), 7.82 (br. d, J=7.7 Hz, 1H), 7.65-7.73 (m, 2H), 7.61 (dt, J=8.7, 3.4 Hz, 1H), 7.35-7.49 (m, 2H), 6.90 (br. d, J=5.9 Hz, 1H), 4.73 (s, 2H), 2.56-2.63 (m, 2H), 2.49-2.53 (m, 2H), 2.22-2.55 (m, 8H), 2.14 (s, 3H).

### Example 197: N-(4-{[6-(5-chloro-2-fluorophenyl)-3-{[(3-methyl-2-oxooxolan-3-yl)methyl]sulfanyl}pyridazin-4-yl]amino}pyridin-2-yl)-3-(4-methylpiperazin-1-yl)propanamide

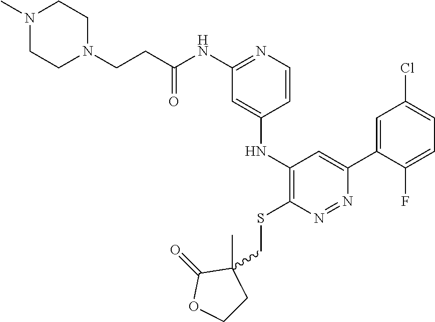 [SMILES: CN1CCN(CCC(=O)Nc2cc(Nc3cc(-c4cc(Cl)ccc4F)nnc3SCC3(C)CCOC3=O)ccn2)CC1]

Example 197 was prepared following the procedure used for the synthesis of Example 115, starting from Intermediate 2 (26 mg, 0.07 mmol) to afford title compound (5 mg, 0.008 mmol, 12% yield). LC-MS (ESI): m/z (M+1): 614.5 (Method 4)

¹H NMR (500 MHz, Chloroform-d) δ ppm 11.10 (br. s, 1H), 8.23 (d, J=5.6 Hz, 1H), 8.15 (dd, J=6.7, 2.7 Hz, 1H), 8.08 (d, J=1.9 Hz, 1H), 7.73 (d, J=1.0 Hz, 1H), 7.36-7.43 (m, 1H), 7.13 (dd, J=10.5, 8.9 Hz, 1H), 6.88 (dd, J=5.6, 2.1 Hz, 1H), 6.40 (s, 1H), 4.42 (td, J=8.9, 4.0 Hz, 1H), 4.31 (td, J=8.8, 7.3 Hz, 1H), 3.99 (d, J=13.7 Hz, 1H), 3.83 (d, J=13.7 Hz, 1H), 2.76-2.81 (m, 2H), 2.55-2.60 (m, 2H), 2.48-2.55 (m, 1H), 2.46-2.98 (m, 8H), 2.43 (br. s, 3H), 2.10-2.20 (m, 1H), 1.48 (s, 3H).

### Example 198 (Enantiomer 1) and Example 199 (Enantiomer 2): N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)propanamide

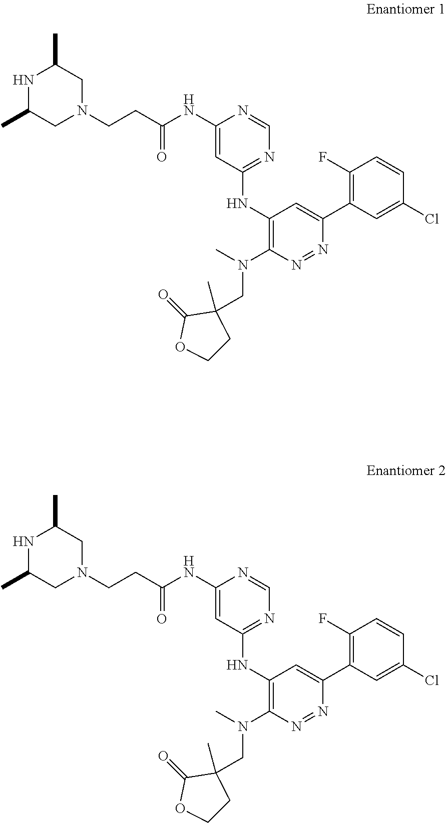 [SMILES: C[C@@H]1CN(CCC(=O)Nc2cc(Nc3cc(-c4cc(Cl)ccc4F)nnc3N(C)CC3(C)CCOC3=O)ncn2)C[C@H](C)N1 ; C[C@@H]1CN(CCC(=O)Nc2cc(Nc3cc(-c4cc(Cl)ccc4F)nnc3N(C)CC3(C)CCOC3=O)ncn2)C[C@H](C)N1]

Racemate N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)propanamide (86 mg, 0.14 mmol, 92% yield)) was prepared following the procedure used for the synthesis of Example 23 starting from Intermediate 351 (108 mg, 0.15 mmol). It was separated into the single enantiomers by preparative chiral HPLC.

### Conditions:

| Column | Chiralpak IC (25 × 2.0 cm), 5μ | |
|---|---|---|
| Mobile phase | n-Hexane/(2-propanol + 0.1% | |
| | isopropylamine) 40/60% v/v | |
| Flow rate (ml/min) | 17 | ml/min |
| DAD detection | 220 | nm |
| Loop | 800 | μL |

Example 198 was obtained as first eluted enantiomer (30 mg)

Rt.=26.7 min, ee >99.9%; LC-MS (ESI): m/z (M+1): 626.3 (Method 3)

¹H NMR (500 MHz, DMSO-d₆) δ ppm 11.06 (s, 1H), 9.22 (br. s, 1H), 8.46-8.49 (m, 1H), 8.46 (s, 1H), 7.99 (dd, J=6.6, 2.7 Hz, 1H), 7.84 (s, 1H), 7.55-7.63 (m, 1H), 7.44 (dd, J=10.4, 9.1 Hz, 1H), 4.18-4.35 (m, 2H), 3.96 (br. d, J=14.5 Hz, 1H), 3.61 (d, J=14.3 Hz, 1H), 2.93 (s, 3H), 2.68-2.82 (m, 4H), 2.52-2.62 (m, 4H), 2.38 (dt, J=12.6, 8.4 Hz, 1H), 1.87-1.98 (m, 1H), 1.69-2.19 (m, 1H), 1.49 (t, J=10.7 Hz, 2H), 1.13 (s, 3H), 0.92 (d, J=6.3 Hz, 6H).

Example 199 was obtained as second eluted enantiomer (32 mg)

Rt.=31.4 min, ee 94.2%; LC-MS (ESI): m/z (M+1): 626.3 (Method 3)

### Example 200: N-(4-{[6-(5-chloro-2-fluorophenyl)-3-({[3-(methoxymethyl)-2-oxooxolan-3-yl]methyl}(methyl)amino)pyridazin-4-yl]amino}pyridin-2-yl)-3-(4-methylpiperazin-1-yl)propanamide

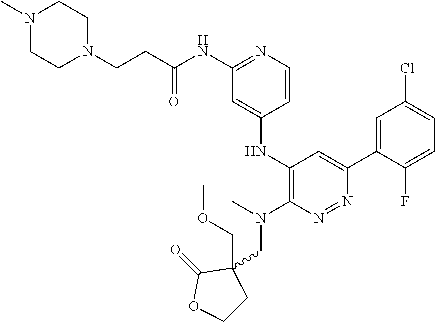 [SMILES: COCC1(CN(C)c2nnc(-c3cc(Cl)ccc3F)cc2Nc2ccnc(NC(=O)CCN3CCN(C)CC3)c2)CCOC1=O]

Example 200 was prepared following the procedure used for the synthesis of Example 115, starting from Intermediate 357 (100 mg, 0.25 mmol) and Intermediate 2 (105 mg, 0.30 mmol) to afford title compound (50 mg, 0.08 mmol, 31% yield). LC-MS (ESI): m/z (M+1): 641.1 (Method 4)

¹H NMR (500 MHz, DMSO-d₆) δ ppm 10.58 (s, 1H), 8.70 (s, 1H), 8.10 (d, J=5.8 Hz, 1H), 7.99 (dd, J=6.6, 2.7 Hz, 1H), 7.91 (d, J=1.1 Hz, 1H), 7.66 (d, J=0.8 Hz, 1H), 7.49-7.60 (m, 1H), 7.41 (dd, J=10.6, 8.9 Hz, 1H), 6.80 (dd, J=5.6, 2.1 Hz, 1H), 4.14-4.29 (m, 2H), 3.99 (d, J=14.3 Hz, 1H), 3.55 (d, J=14.5 Hz, 1H), 3.36-3.46 (m, 2H), 3.17 (s, 3H), 2.94 (s, 3H), 2.55-2.62 (m, 2H), 2.50 (s, 2H), 2.18-2.33 (m, 2H), 2.17-2.49 (m, 8H), 2.14 (s, 3H).

### Example 201 (Enantiomer 1) and Example 202 (Enantiomer 2): N-(4-{[6-(5-chloro-2-fluorophenyl)-3-({[3-(methoxymethyl)-2-oxooxolan-3-yl]methyl}(methyl)amino)pyridazin-4-yl]amino}pyridin-2-yl)-3-(4-methylpiperazin-1-yl)propanamide

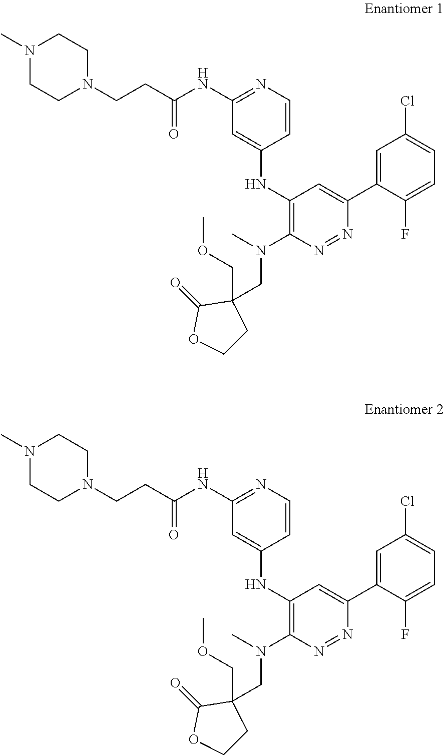 [SMILES: COCC1(CN(C)c2nnc(-c3cc(Cl)ccc3F)cc2Nc2ccnc(NC(=O)CCN3CCN(C)CC3)c2)CCOC1=O ; COCC1(CN(C)c2nnc(-c3cc(Cl)ccc3F)cc2Nc2ccnc(NC(=O)CCN3CCN(C)CC3)c2)CCOC1=O]

Racemic N-(4-{[6-(5-chloro-2-fluorophenyl)-3-({[3-(methoxymethyl)-2-oxooxolan-3-yl]methyl}(methyl)amino)pyridazin-4-yl]amino}pyridin-2-yl)-3-(4-methylpiperazin-1-yl)propanamide (Example 200, 43 mg, 0.07 mmol) was separated into the single enantiomers by preparative chiral HPLC.

### Conditions:

| Column | Chiralpak IC (25 × 2.0 cm), 5μ | |
|---|---|---|
| Mobile phase | n-Hexane/(Ethanol + 0.1% | |
| | isopropylamine) 45/55% v/v | |
| Flow rate (ml/min) | 17 | ml/min |
| DAD detection | 220 | nm |
| Loop | 800 | μL |

Example 201 was obtained as first eluted enantiomer (16 mg)

Rt.=17 min, ee >99.9%; LC-MS (ESI): m/z (M+1): 641.1 (Method 4)

Example 202 was obtained as second eluted enantiomer (16 mg)

Rt.=19.8 min. ee 92.2%; LC-MS (ESI): m/z (M+1): 641.1 (Method 4)

### Example 203: 4-({[6-(5-chloro-2-fluorophenyl)-4-({2-[3-(4-methylpiperazin-1-yl)propanamido]pyridin-4-yl}amino)pyridazin-3-yl]sulfanyl}methyl)benzoic acid

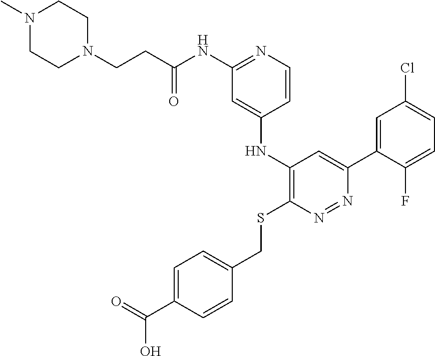 [SMILES: CN1CCN(CCC(=O)Nc2cc(Nc3cc(-c4cc(Cl)ccc4F)nnc3SCc3ccc(C(=O)O)cc3)ccn2)CC1]

Tetrabutylammonium fluoride 1M in THE (0.15 mL, 0.15 mmol) was added to a solution of Intermediate 314 (80 mg, 0.13 mmol) in THE (3 mL). The mixture was stirred at RT overnight, then 4-(bromomethyl)benzoic acid (28 mg, 0.13 mmol) was added and the reaction stirred at RT for 1 h. The mixture was diluted with EtOAc and 10% citric acid aqueous solution, phases were separated, the organic phase was discarded, the aqueous one was concentrated under vacuum and the residue was purified by reverse flash chromatography on Biotage C18 cartridge (from H₂O+0.1% NH₄OH to 40% MeCN) to afford title compound (40 mg, 0.06 mmol, 47% yield). LC-MS (ESI): m/z (M+1): 636.5 (Method 4)

¹H NMR (400 MHz, DMSO-d₆) δ ppm 10.59 (s, 1H), 8.92 (br. s, 1H), 8.10 (d, J=5.7 Hz, 1H), 7.97-8.04 (m, 2H), 7.90 (d, J=8.1 Hz, 2H), 7.67 (s, 1H), 7.57-7.64 (m, 3H), 7.38-7.47 (m, 1H), 6.90 (dd, J=5.6, 1.6 Hz, 1H), 4.74 (s, 2H), 2.55-2.65 (m, 2H), 2.47-2.53 (m, 2H), 2.21-2.48 (m, 8H), 2.15 (s, 3H).

### Example 204: N-(4-{[6-(5-chloro-2-fluorophenyl)-3-{[(6-oxooxan-2-yl)methyl]sulfanyl}pyridazin-4-yl]amino}pyridin-2-yl)-3-(4-methylpiperazin-1-yl)propanamide

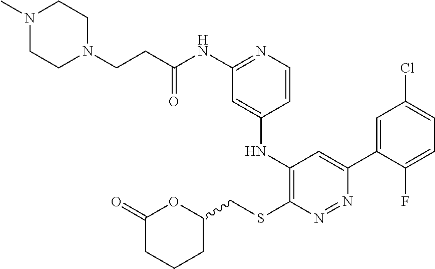 [SMILES: CN1CCN(CCC(=O)Nc2cc(Nc3cc(-c4cc(Cl)ccc4F)nnc3SCC3CCCC(=O)O3)ccn2)CC1]

Example 204 was prepared following the procedure used for the synthesis of Example 203, starting from Intermediate 314 (166 mg, 0.28 mmol) and 6-(iodomethyl)oxan-2-one (Intermediate 358, 66 mg, 0.28 mmol) to afford title compound (90 mg, 0.15 mmol, 53% yield). LC-MS (ESI): m/z (M+1): 614.3 (Method 4)

¹H NMR (500 MHz, DMSO-d₆) δ ppm 10.61 (s, 1H), 8.98 (br. s, 1H), 8.11 (d, J=5.6 Hz, 1H), 8.04 (br. s, 1H), 7.99 (dd, J=6.4, 2.6 Hz, 1H), 7.67 (br. s, 1H), 7.61 (dt, J=8.4, 3.5 Hz, 1H), 7.43 (t, J=9.7 Hz, 1H), 6.92 (br. d, J=4.7 Hz, 1H), 4.64-4.76 (m, 1H), 3.73-3.83 (m, 1H), 3.61-3.72 (m, 1H), 2.52-2.62 (m, 5H), 2.34-2.42 (m, 1H), 2.18-2.48 (m, 8H), 2.14 (s, 3H), 2.01-2.10 (m, 1H), 1.78-1.88 (m, 2H), 1.66 (dtd, J=13.7, 10.3, 6.4 Hz, 1H).

### Example 205: N-(2-{[6-(5-chloro-2-fluorophenyl)-4-({2-[3-(4-methylpiperazin-1-yl)propanamido]pyridin-4-yl}amino)pyridazin-3-yl]sulfanyl}ethyl)-5-oxooxolane-3-carboxamide

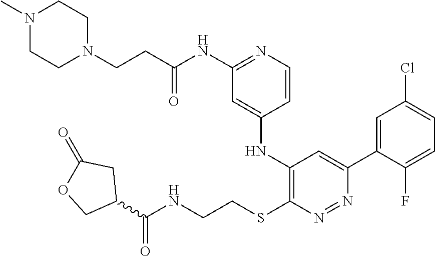 [SMILES: CN1CCN(CCC(=O)Nc2cc(Nc3cc(-c4cc(Cl)ccc4F)nnc3SCCNC(=O)C3COC(=O)C3)ccn2)CC1]

To a solution of Intermediate 362 (50 mg, 0.09 mmol) and tetrahydro-5-oxo-3-furoic acid (13 mg, 0.10 mmol) in DCM (0.9 ml), DIPEA (0.04 ml, 0.23 mmol) and HATU (38 mg, 0.10 mmol) were added. The reaction was stirred at RT for 30 min. The reaction was diluted with DCM and washed with saturated aqueous NaHCO₃ solution. The organic phase was dried over Na₂SO₄, filtered and concentrated under vacuum. The residue material was purified by flash chromatography on Biotage NH cartridge (from DCM to 3% MeOH), then by reverse flash chromatography on Biotage C18 cartridge (from H₂O+0.1% NH₄OH to 80% MeCN) to afford title compound (20 mg, 0.03 mmol, 33% yield). LC-MS (ESI): m/z (M+1): 657.3 (Method 4)

¹H NMR (500 MHz, DMSO-d₆) δ ppm 10.61 (s, 1H), 8.89 (s, 1H), 8.47 (br. s, 1H), 8.08-8.14 (m, 1H), 8.04 (br. s, 1H), 7.99 (dd, J=6.5, 2.7 Hz, 1H), 7.67 (br. s, 1H), 7.54-7.63 (m, 1H), 7.43 (dd, J=10.4, 9.0 Hz, 1H), 6.92 (br. d, J=4.3 Hz, 1H), 4.40 (t, J=8.4 Hz, 1H), 4.22 (dd, J=8.9, 5.4 Hz, 1H), 3.41-3.52 (m, 4H), 3.30-3.35 (m, 1H), 2.65-2.72 (m, 1H), 2.55-2.62 (m, 3H), 2.48-2.55 (m, 2H), 2.17-2.47 (m, 8H), 2.14 (s, 3H).

### Example 206: (1-methylpiperidin-4-yl)methyl 3-({[6-(5-chloro-2-fluorophenyl)-4-({1H-pyrrolo[2,3-b]pyridin-4-yl}amino)pyridazin-3-yl]sulfanyl}methyl)benzoate

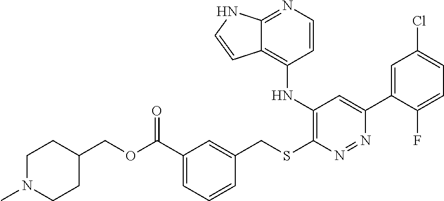 [SMILES: CN1CCC(COC(=O)c2cccc(CSc3nnc(-c4cc(Cl)ccc4F)cc3Nc3ccnc4[nH]ccc34)c2)CC1]

Example 206 was prepared following the procedure used for the synthesis of Example 23 starting from Intermediate 368 (30 mg, 0.04 mmol) to afford title compound (10 mg, 0.016 mmol. 39% yield). LC-MS (ESI): m/z (M+1): 617.3 (Method 4)

¹H NMR (400 MHz, DMSO-d₆) δ ppm 11.67 (br s, 1H), 8.75 (s, 1H), 8.07-8.14 (m, 2H), 7.95 (dd, J=6.6, 2.7 Hz, 1H), 7.86 (d, J=7.8 Hz, 1H), 7.80 (d, J=7.8 Hz, 1H), 7.53-7.61 (m, 1H), 7.50 (t, J=7.7 Hz, 1H), 7.33-7.34 (m, 1H), 7.31-7.40 (m, 1H), 7.22 (s, 1H), 6.88 (d, J=5.3 Hz, 1H), 6.13 (dd, J=3.3, 1.9 Hz, 1H), 4.78 (s, 2H), 4.10 (d, J=5.9 Hz, 2H), 2.68 (br d, J=11.1 Hz, 2H), 2.08 (s, 3H), 1.76 (br t, J=10.9 Hz, 2H), 1.56-1.68 (m, 3H), 1.16-1.31 (m, 2H).

### Example 207: 2-(dimethylamino)ethyl 3-({[6-(5-chloro-2-fluorophenyl)-4-({1H-pyrrolo[2,3-b]pyridin-4-yl}amino)pyridazin-3-yl]sulfanyl}methyl)benzoate

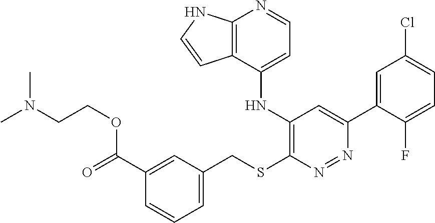 [SMILES: CN(C)CCOC(=O)c1cccc(CSc2nnc(-c3cc(Cl)ccc3F)cc2Nc2ccnc3[nH]ccc23)c1]

A solution of DIPEA (0.04 mL, 0.24 mmol), 2-(dimethylamino)ethanol (14 mg, 0.16 mmol) and Intermediate 367 (40 mg, 0.08 mmol) in DMF (1.6 ml) was treated with HATU (60 mg, 0.16 mmol). The mixture was stirred 4 hrs at 50° C. The mixture was diluted with EtOAc, washed with saturated aqueous NaHCO₃ solution and brine. The organic phase was separated, dried over Na₂SO₄, filtered and concentrated under reduced pressure. The crude product was purified by flash chromatography on Biotage silica NH cartridge (from cHex to 5% EtOAc/MeOH 10/1) and then by flash chromatography on Biotage silica cartridge (from DCM to 1% of MeOH) to afford title compound (11 mg, 0.02 mmol, 24% yield). LC-MS (ESI): m/z (M+1): 577.3 (Method 4)

¹H NMR (400 MHz, DMSO-d₆) δ ppm 11.67 (br. s, 1H), 8.76 (s, 1H), 8.06-8.15 (m, 2H), 7.96 (dd, J=6.5, 2.6 Hz, 1H), 7.85 (d, J=7.7 Hz, 1H), 7.80 (d, J=7.8 Hz, 1H), 7.54-7.61 (m, 1H), 7.50 (t, J=7.7 Hz, 1H), 7.29-7.40 (m, 2H), 7.21 (s, 1H), 6.88 (d, J=5.4 Hz, 1H), 6.14 (dd, J=3.3, 1.9 Hz, 1H), 4.78 (s, 2H), 4.34 (t, J=5.8 Hz, 2H), 2.58 (t, J=5.7 Hz, 2H), 2.17 (s, 6H).

### Example 208 (Enantiomer 1) and Example 209 (Enatiomer 2): N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide

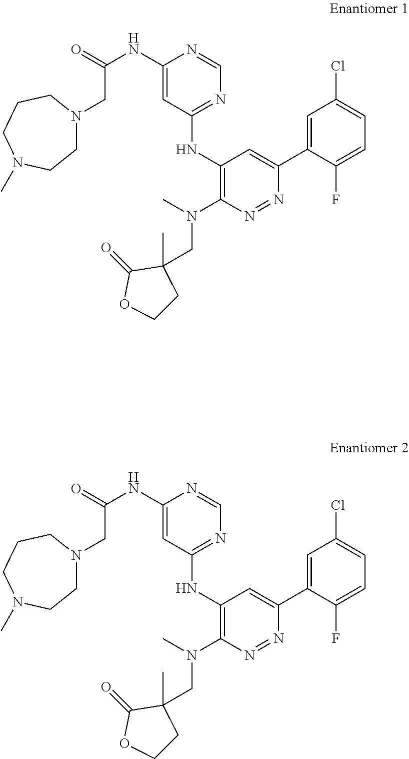 [SMILES: CN1CCCN(CC(=O)Nc2cc(Nc3cc(-c4cc(Cl)ccc4F)nnc3N(C)CC3(C)CCOC3=O)ncn2)CC1 ; CN1CCCN(CC(=O)Nc2cc(Nc3cc(-c4cc(Cl)ccc4F)nnc3N(C)CC3(C)CCOC3=O)ncn2)CC1]

Racemate mixture of N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide (169 mg, 0.27 mmol, 68% yield) was prepared following the procedure used for the synthesis of Example 155, starting from Intermediate 372 (240 mg, 0.41 mmol) and formaldehyde 37% w/w in water (46 μl, 51 mmol). It was separated into the single enantiomers by preparative chiral HPLC.

### Conditions:

| Column | Chiralcel OD-H (25 × 2.0 cm), 5μ | |
|---|---|---|
| Mobile phase | n-Hexane/(Ethanol + 0.1% | |
| | isopropylamine) 50/50% v/v | |
| Flow rate (ml/min) | 17 | ml/min |
| DAD detection | 220 | nm |
| Loop | 800 | μL |

Example 208 was obtained as first eluted enantiomer (65 mg)

Rt.=7.5 min, ee >99.9%; LC-MS (ESI): m/z (M+1): 612.4 (Method 4)

¹H NMR (500 MHz, Chloroform-d) δ ppm 9.75 (br. s, 1H), 9.10 (d, J=1.5 Hz, 1H), 8.64 (s, 1H), 8.40 (s, 1H), 8.14 (dd, J=6.6, 2.7 Hz, 1H), 7.94 (d, J=0.8 Hz, 1H), 7.39 (ddd, J=8.7, 4.1, 2.8 Hz, 1H), 7.16 (dd, J=10.4, 8.9 Hz, 1H), 4.25-4.36 (m, 2H), 3.78-3.87 (m, 1H), 3.68-3.76 (m, 1H), 3.34 (s, 2H), 2.91 (s, 3H), 2.85-2.95 (m, 4H), 2.69-2.83 (m, 4H), 2.46 (s, 3H), 2.19-2.30 (m, 1H), 1.99-2.08 (m, 1H), 1.89-1.98 (m, 2H), 1.25 (s, 3H).

Example 209 was obtained as the second eluted enantiomer (67 mg)

Rt.=9.5 min, ee 90.2%; LC-MS (ESI): m/z (M+1): 612.4 (Method 4)

### Comparative Newly Synthesised Compounds Lacking a Pyrimidinyl, a Pyridinyl or a Pyridinyl Condensed Group Linked to the Amino Group Bearing the Pyridazine Ring

### Example C1: [6-(5-Chloro-2-fluorophenyl)-3-methoxypyridazin-4-yl]-1,3-benzothiazol-6-amine

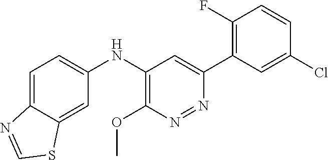 [SMILES: COc1nnc(-c2cc(Cl)ccc2F)cc1Nc1ccc2ncsc2c1]

Example C1 was prepared following the procedure used for the synthesis of Example 1, starting from 6-(5-chloro-2-fluorophenyl)-3-methoxypyridazin-4-amine (Intermediate 37, 80 mg, 0.31 mmol) and 6-bromobenzothiazole (81 mg, 0.38 mmol) to afford title compound (14 mg, 0.04 mmol, 11% yield).

LC-MS (ESI): m/z (M+1): 387.3 (Method 1)

¹H NMR (400 MHz, Chloroform-d) δ ppm 9.00 (s, 1H), 8.19 (d, J=8.69 Hz, 1H), 8.08 (dd, J=6.71, 2.75 Hz, 1H), 7.89 (d, J=2.09 Hz, 1H), 7.41-7.49 (m, 2H), 7.35 (ddd, J=8.78, 4.21, 2.75 Hz, 1H), 7.08 (dd, J=10.56, 8.80 Hz, 1H), 6.79 (s, 1H), 4.29-4.36 (m, 3H).

### Pharmacological Activity of the Compounds of the Invention

### In Vitro Assay

The enzymatic activity of compounds of the present invention was monitored measuring the formation of ADP using the ADP-GLO Kinases assay. Following the incubation of the purified enzyme, a substrate and ATP, the produced ADP was converted into ATP, which in turn was converted into light by Ultra-Glo Luciferase. The luminescent signal positively correlated with ADP amount and kinase activity. Briefly, the kinase reaction was performed by incubating 2.6 nM of the purified, commercially available human ALK5 (recombinant TGF β1 N-term GST-tagged, 80-end), a final concentration of TGFβ1 peptide 94.5 μM (Promega, T36-58) and ultra-pure ATP (Promega V915B). The ATP concentration was set at the Km value (concentration of substrate which permits the enzyme to achieve half maximal velocity (Vmax)) of ALK5 (0.5 μM). Compound and ALK5 kinase were mixed and incubated for 15 mins. Reactions were initiated by addition of ATP at a final concentration in the assay of 0.83 μM. After an incubation of 120 min, the reaction was stopped, and ADP production detected with ADP-Glo kit according to manufacturer's indications. To overcome the assay wall limit for very potent compounds the assay protocol was changed by using a high ATP concentration (30-fold Km). Compounds and ALK5 kinase were mixed for 15 min and the reaction initiated by addition of TGFβ1 peptide and ATP at a final concentration in the assay of 15 μM. After an incubation of 60 min, the kinase reaction was stopped, and ADP production detected with ADP-Glo kit according to manufacturer's indications. All reaction and incubation steps were performed at 25° C. and the assays were performed in 384-well format and validated using a selection of reference compounds tested in 11-point concentration-response curve. The results for individual compounds are provided below in Table 4 wherein the compounds are classified in term of potency with respect to their inhibitory activity on ALK5 receptor. Results were expressed as pIC₅₀ (negative logarithm of IC₅₀) and subsequently converted to pKᵢ (negative logarithm of dissociate function Kᵢ) using the Cheng-Prusoff equation. The higher the value of pKᵢ, the greater the inhibition of ALK5 activity. As it can be appreciated, all the compounds of Table 4 show pKᵢ values greater than 8.5 when tested in the biochemical ALK5 assay.

TABLE 4

| Example No | ALK5 pKᵢ |
|---|---|
| Compounds of formula (I) wherein A corresponds to A1: 1, 7, 9, 13, | ≥10 |
| 14, 16, 20, 21, 22, 24, 25, 26, 27, 28, 29, 30, 31, 33, 34, 36, 38, 45, | |
| 47, 50, 54, 60, 61, 62, 71, 72, 73, 74, 75, 76, 77, 78, 79, 80, 81, 82, | |
| 83, 84, 85, 86, 87, 88, 89, 90, 92, 94, 95, 96, 97, 101, 105, 107, 108, | |
| 109, 110, 111, 112, 115, 117, 118, 119, 120, 121, 122, 124, 126, 127, | |
| 128, 129, 130, 131, 132, 135, 140, 141, 142, 146, 147, 148, 149, 152, | |
| 153, 154, 156, 157, 158, 159, 160, 161, 162, 163, 164, 166, 168, 169, | |
| 170, 171, 172, 173, 174, 175, 178, 180, 188, 189, 190, 193, 194, 200, | |
| 201, 203, 204, 205 | |
| Compounds of formula (I) wherein A corresponds to A2: 2, 3, 6, 32, | |
| 46, 51 | |
| Compounds of formula (I) wherein A corresponds to A4: 137, 138, | |
| 139, 144, 176, 177, 181, 185, 186, 187, 198, 199, 208, 209 | |
| 4, 5, 8, 12, 15, 17, 18, 23, 35, 40, 44, 48, 52, 55, 68, 91, 93, 98, 99, | 9.5-9.9 |
| 100, 102, 106, 113, 114, 116, 123, 125, 133, 134, 136, 143, 145, 155, | |
| 165, 167, 179, 182, 183, 191, 192, 195, 196, 197, 202, 206, 207 | |
| 10, 11, 19, 37, 39, 40, 41, 42, 43, 49, 53, 56, 57, 58, 69, 70, 150, 151, | 8.5-9.4 |
| 184 | |

### Comparative Examples

Compound of the example C1 was tested in the same in vitro assay described above and showed a value of pKᵢ, lower than 8.5 (Table 5).

TABLE 5

| Example No | ALK5 pKᵢ |
|---|---|
| C1 | 8.2 |

This data demonstrates that, conversely to the compound C1 characterized by lacking a pyrimidinyl, a pyridinyl or a pyridinyl condensed group linked to the amino group bearing the pyridazine ring, the presence of a pyridinyl or a pyridinyl condensed group linked to the amino group bearing the pyridazine ring in the present invention compounds unexpectedly and remarkably determines a relevant increase in the inhibitory activity on the ALK5 receptor.

1. A compound of general formula (I) or a pharmaceutically acceptable salt thereof: 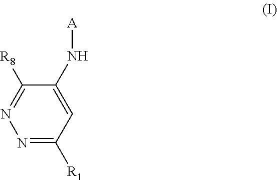
wherein:
A is selected from the group consisting of A1, A2, A3 and A4 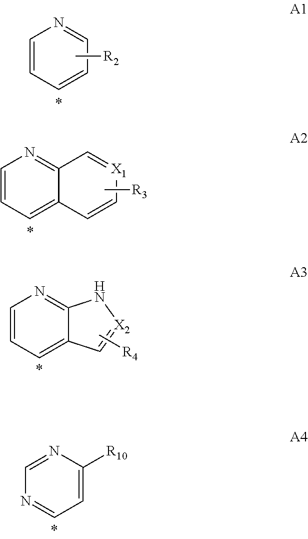
R₁ is selected from the group consisting of aryl and pyridyl, wherein the aryl and pyridyl are optionally substituted by one or more groups selected from the group consisting of a halogen atom and —(C₁-C₆)alkyl;
R₂ is selected from the group consisting of —NR₅C(O)R₆, —NR₅R₉ and —NH₂;
X₁ is C or CH;
X₂ is C, CH or N;
R₃ is —OR₇;
R₄ is H or —C(O)O—(C₁-C₆)alkyl;
R₅ is H or —(C₁-C₆)alkyl;
R₆ is selected from the group consisting of:
—(C₃-C₉)heterocycloalkyl substituted by one or more —(C₁-C₆)alkyl;
—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl and —(C₃-C₆)cycloalkyl;
—(C₁-C₆)alkylene-NH₂;
—(C₃-C₆)cycloalkyl optionally substituted by one or more —(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and —(C₃-C₆)cycloalkyl; and
—(C₃-C₆)cycloalkyl optionally substituted by one or more —(C₃-C₉)heterocycloalkyl, wherein the —(C₃-C₉)heterocycloalkyl is optionally substituted by one or more groups selected from the group consisting of —(C₁-C₆)alkyl, —(C₁-C₆)alkylene-OH, —O—(C₁-C₆)alkyl, —C(O)OH, —C(O)O—(C₁-C₆)alkyl, —(C₁-C₆)haloalkyl, —(C₃-C₆)cycloalkyl and a halogen atom;
R₇ is selected from the group consisting of —(C₁-C₆)alkyl and —(C₁-C₆)alkylene-(C₃-C₉)heterocycloalkyl, wherein the —(C₃-C₉)heterocycloalkyl is optionally substituted by one or more —(C₁-C₆)alkyl;
R₈ is selected from the group consisting of:
—NR_{A}R_{B};
—SH;
—S—(C₁-C₆)alkyl, wherein the —(C₁-C₆)alkyl is optionally substituted by one or more —OH;
—S—(C₁-C₆)alkylene-OH;
—S—(C₃-C₉)heterocycloalkyl, wherein the —(C₃-C₉)heterocycloalkyl is optionally substituted by one or more groups selected from the group consisting of —(C₁-C₆)alkyl and oxo;
—S—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
—S(O)=NH—(C₁-C₆)alkyl;
—S(O)₂—(C₁-C₆)alkyl;
—S(O)—(C₁-C₆)alkyl;
—S—(C₁-C₆)alkylene-(C₃-C₆)cycloalkyl, wherein the —(C₃-C₆)cycloalkyl is optionally substituted by one or more groups selected from the group consisting of —(C₁-C₆)alkyl, —(C₁-C₆)alkylene-OH and —OH;
S—(C₁-C₆)alkylene-aryl, wherein the aryl is optionally substituted by one or more groups selected from the group consisting of —C(O)OH, —C(O)O—(C₁-C₆)alkylene-NR_{A}R_{C} and —C(O)O—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
—S—(C₁-C₆)alkylene-Si((C₁-C₆)alkyl)₃;
—S—(C₁-C₆)alkylene-O—(C₁-C₆)alkylene-OH;
—S—(C₁-C₆)alkylene-O—(C₁-C₆)alkylene-(C₃-C₉)heterocycloalkyl, wherein the —(C₃-C₉)heterocycloalkyl is optionally substituted by one or more groups selected from the group consisting of oxo and —(C₁-C₆)alkyl;
—S—(C₁-C₆)alkylene-NH—C(O)—(C₃-C₉)heterocycloalkyl, wherein the —(C₃-C₉)heterocycloalkyl is optionally substituted by one or more oxo;
—S—(C₁-C₆)alkylene-NH—(C₃-C₉)heterocycloalkyl, wherein the —(C₃-C₉)heterocycloalkyl is optionally substituted by one or more oxo;
—O—(C₁-C₆)alkyl;
—O—(C₁-C₆)haloalkyl;
—O—(C₁-C₆)alkylene-OH, wherein the —O—(C₁-C₆)alkylene is substituted by one or more —OH;
—O—(C₁-C₆)alkylene-C(O)O—(C₁-C₆)alkyl;
—O—(C₁-C₆)alkylene-NR_{A}R_{B};
—O—(C₁-C₆)alkylene-N⁺R_{A}R_{B}R_{C};
—O—(C₁-C₆)alkylene-S—(C₁-C₆)alkyl;
—O—(C₁-C₆)alkylene-S(O)—(C₁-C₆)alkyl;
—O—(C₁-C₆)alkylene-S(O)₂—(C₁-C₆)alkyl;
—O—(C₁-C₆)alkylene-NH—S(O)₂—(C₁-C₆)alkyl;
—O—(C₁-C₆)alkylene-O—(C₁-C₆)alkyl;
—O—(C₁-C₆)alkylene-(C₃-C₆)cycloalkyl, wherein the —(C₃-C₆)cycloalkyl is optionally substituted by one or more groups selected from the group consisting of —(C₁-C₆)alkyl, —(C₁-C₆)alkylene-OH, —C(O)O—(C₁-C₆)alkyl and —OH;
—O—(C₃-C₆)cycloalkyl, wherein the —(C₃-C₆)cycloalkyl is optionally substituted by one or more groups selected from the group consisting of —(C₁-C₆)alkylene-OH and —OH;
—O—(C₁-C₆)alkylene-aryl, wherein the aryl is optionally substituted by one or more —OH;
—O—(C₁-C₆)alkylene-aryl, wherein the aryl is fused to a —(C₅-C₆)heterocycloalkyl, wherein the —(C₅-C₆)heterocycloalkyl is optionally substituted by one or more groups selected from the group consisting of oxo and —(C₁-C₆)alkyl;
—O—(C₃-C₉)heterocycloalkyl; and
—O—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
R₉ is a heteroaryl optionally substituted by one or more groups selected from the group consisting of —C(O)O—(C₁-C₆)alkyl and —(C₃-C₉)heterocycloalkyl, wherein the —(C₃-C₉)heterocycloalkyl is optionally substituted by one or more —(C₁-C₆)alkyl;
R₁₀ is —NR₅C(O)R₆;
R_{A} is H or —(C₁-C₆)alkyl;
R_{B} is selected from the group consisting of:
H;
—(C₁-C₆)alkyl optionally substituted by one or more groups selected from the group consisting of a halogen atom and —OH;
—S(O)₂—(C₁-C₆)alkyl;
—(C₁-C₆)alkylene-aryl, wherein the aryl is susbtituted by —OH;
—(C₁-C₆)alkylene-OH;
—(C₃-C₉)heterocycloalkyl;
—(C₁-C₆)alkylene-C(O)O—(C₁-C₆)alkyl;
—(C₁-C₆)alkylene-aryl-OCO—(C₁-C₆)alkyl; and
—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl, —(C₁-C₆)alkylene-OH, —(C₁-C₆)alkylene-O—(C₁-C₆)alkyl, —(C₁-C₆)haloalkyl and oxo; or alternatively
R_{A} and R_{B} together with the nitrogen atom to which they are attached form a —(C₃-C₆)heterocycloalkyl, wherein the —(C₃-C₆)heterocycloalkyl is optionally substituted by one or more groups selected from the group consisting of —C(O)OH, —(C₁-C₆)alkylene-OH, —C(O)O—(C₁-C₆)alkyl and oxo, or the —(C₃-C₆)heterocycloalkyl is optionally substituted on two adjacent carbon atoms forming an additional condensed —(C₅-C₆)heterocycloalkyl, optionally substituted by oxo; and
R_{C} is —(C₁-C₆)alkyl.

2. The compound or salt according to claim 1, wherein A is a group A1 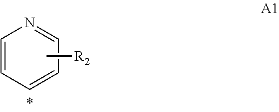
according to formula (Ia); 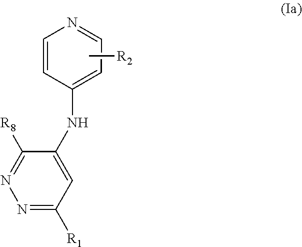
wherein:
R₁ is selected from the group consisting of aryl and pyridyl, wherein the aryl and pyridyl are optionally substituted by one or more groups selected from the group consisting of —(C₁-C₆)alkyl and a halogen atom;
R₂ is selected from the group consisting of —NR₅C(O)R₆, —NR₅R₉ and —NH₂;
R₅ is H or —(C₁-C₆)alkyl;
R₆ is selected from the group consisting of:
—(C₃-C₉)heterocycloalkyl substituted by one or more —(C₁-C₆)alkyl;
—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl, —(C₁-C₆)alkylene-NH—C(O)O—(C₁-C₆)alkyl, —(C₁-C₆)haloalkyl, —C(O)O—(C₁-C₆)alkyl and —(C₃-C₆)cycloalkyl;
—(C₁-C₆)alkylene-NH₂;
—(C₃-C₆)cycloalkyl optionally substituted by one or more —(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and —(C₃-C₆)cycloalkyl; and
—(C₃-C₆)cycloalkyl optionally substituted by one or more —(C₃-C₉)heterocycloalkyl, wherein the —(C₃-C₉)heterocycloalkyl is optionally substituted by one or more groups selected from the group consisting of —(C₁-C₆)alkyl, —(C₁-C₆)alkylene-OH, —O—(C₁-C₆)alkyl, —C(O)OH, —C(O)O—(C₁-C₆)alkyl, —(C₁-C₆)haloalkyl, —(C₃-C₆)cycloalkyl and a halogen atom;
R₈ is selected from the group consisting of:
—NR_{A}R_{B};
—SH;
—S—(C₁-C₆)alkyl, wherein the —(C₁-C₆)alkyl is optionally substituted by one or more —OH;
—S—(C₁-C₆)alkylene-OH;
—S—(C₃-C₉)heterocycloalkyl, wherein the —(C₃-C₉)heterocycloalkyl is optionally substituted by one or more groups selected from the group consisting of —(C₁-C₆)alkyl and oxo;
—S—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
—S(O)=NH—(C₁-C₆)alkyl;
—S(O)₂—(C₁-C₆)alkyl;
—S(O)—(C₁-C₆)alkyl;
—S—(C₁-C₆)alkylene-(C₃-C₆)cycloalkyl, wherein the —(C₃-C₆)cycloalkyl is optionally substituted by one or more groups selected from the group consisting of —(C₁-C₆)alkyl, —(C₁-C₆)alkylene-OH and —OH;
S—(C₁-C₆)alkylene-aryl, wherein the aryl is optionally substituted by one or more groups selected from the group consisting of —C(O)OH, —C(O)O—(C₁-C₆)alkylene-NR_{A}R_{C} and —C(O)O—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
—S—(C₁-C₆)alkylene-Si((C₁-C₆)alkyl)₃;
—S—(C₁-C₆)alkylene-O—(C₁-C₆)alkylene-OH;
—S—(C₁-C₆)alkylene-O—(C₁-C₆)alkylene-(C₃-C₉)heterocycloalkyl, wherein the —(C₃-C₉)heterocycloalkyl is optionally substituted by one or more groups selected from the group consisting of oxo and —(C₁-C₆)alkyl;
—S—(C₁-C₆)alkylene-NH—C(O)—(C₃-C₉)heterocycloalkyl, wherein the —(C₃-C₉)heterocycloalkyl is optionally substituted by one or more oxo;
—S—(C₁-C₆)alkylene-NH—(C₃-C₉)heterocycloalkyl, wherein the —(C₃-C₉)heterocycloalkyl is optionally substituted by one or more oxo;
—O—(C₁-C₆)alkyl;
—O—(C₁-C₆)haloalkyl;
—O—(C₁-C₆)alkylene-OH, wherein s-aid-the-O—(C₁-C₆)alkylene is substituted by one or more —OH;
—O—(C₁-C₆)alkylene-C(O)O—(C₁-C₆)alkyl;
—O—(C₁-C₆)alkylene-NR_{A}R_{B};
—O—(C₁-C₆)alkylene-N⁺R_{A}R_{B}R_{C};
—O—(C₁-C₆)alkylene-S—(C₁-C₆)alkyl;
—O—(C₁-C₆)alkylene-S(O)—(C₁-C₆)alkyl;
—O—(C₁-C₆)alkylene-S(O)₂—(C₁-C₆)alkyl;
—O—(C₁-C₆)alkylene-NH—S(O)₂—(C₁-C₆)alkyl;
—O—(C₁-C₆)alkylene-O—(C₁-C₆)alkyl;
—O—(C₁-C₆)alkylene-(C₃-C₆)cycloalkyl, wherein the —(C₃-C₆)cycloalkyl is optionally substituted by one or more groups selected from the group consisting of —(C₁-C₆)alkyl, —(C₁-C₆)alkylene-OH, —C(O)O—(C₁-C₆)alkyl and —OH;
—O—(C₃-C₆)cycloalkyl, wherein the —(C₃-C₆)cycloalkyl is optionally substituted by one or more groups selected from the group consisting of —(C₁-C₆)alkylene-OH and —OH;
—O—(C₁-C₆)alkylene-aryl, wherein the aryl is optionally substituted by one or more —OH;
—O—(C₁-C₆)alkylene-aryl, wherein the aryl is fused to a —(C₅-C₆)heterocycloalkyl, wherein the —(C₅-C₆)heterocycloalkyl is optionally substituted by one or more groups selected from the group consisting of oxo and —(C₁-C₆)alkyl;
—O—(C₃-C₉)heterocycloalkyl; and
—O—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
R₉ is a heteroaryl optionally substituted by one or more groups selected from the group consisting of —C(O)O—(C₁-C₆)alkyl and —(C₃-C₉)heterocycloalkyl, wherein the —(C₃-C₉)heterocycloalkyl is optionally substituted by one or more —(C₁-C₆)alkyl;
R_{A} is H or —(C₁-C₆)alkyl;
R_{B} is H or is selected from the group consisting of:
—(C₁-C₆)alkyl optionally substituted by one or more groups selected from the group consisting of a halogen atom and —OH;
—S(O)₂—(C₁-C₆)alkyl;
—(C₁-C₆)alkylene-aryl, wherein the aryl is susbtituted by —OH;
—(C₃-C₉)heterocycloalkyl;
—(C₁-C₆)alkylene-C(O)O—(C₁-C₆)alkyl; and
—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl, —(C₁-C₆)alkylene-OH, —(C₁-C₆)alkylene-O—(C₁-C₆)alkyl and oxo; or alternatively
R_{A} and R_{B} together with the nitrogen atom to which they are attached may form a —(C₃-C₆)heterocycloalkyl, wherein the —(C₃-C₆)heterocycloalkyl is optionally substituted by one or more groups selected from the group consisting of —C(O)OH, —(C₁-C₆)alkylene-OH, —C(O)O—(C₁-C₆)alkyl and oxo; and
R_{C} is —(C₁-C₆)alkyl.

3. The compound or salt according to claim 2, wherein the compound is selected from the group consisting of:
N-(4-{[6-(5-chloro-2-fluorophenyl)-3-(2-hydroxyethoxy)pyridazin-4-yl]amino}pyridin-2-yl)-3-(4-methylpiperazin-1-yl)propanamide;
N-(4-{[6-(5-chloro-2-fluorophenyl)-3-[3-(methylsulfanyl)propoxy]pyridazin-4-yl]amino}pyridin-2-yl)-3-(4-methylpiperazin-1-yl)propanamide;
N-(4-{[6-(5-chloro-2-fluorophenyl)-3-(3-methanesulfonylpropoxy)pyridazin-4-yl]amino}pyridin-2-yl)-3-(4-methylpiperazin-1-yl)propanamide;
N-(4-{[3-(2-aminoethoxy)-6-(5-chloro-2-fluorophenyl)pyridazin-4-yl]amino}pyridin-2-yl)-3-(4-methylpiperazin-1-yl)propanamide;
N-(4-{[6-(5-chloro-2-fluorophenyl)-3-(2-methanesulfonamidoethoxy)pyridazin-4-yl]amino}pyridin-2-yl)-3-(4-methylpiperazin-1-yl)propanamide;
methyl 4-{1[(4-{[6-(5-chloro-2-fluorophenyl)-3-[2-(dimethylamino)ethoxy] pyridazin-4-yl]amino}pyridin-2-yl)carbamoyl]methyl}-1-methylpiperazine-2-carboxylate;
N-(4-{[6-(5-chloro-2-fluorophenyl)-3-methoxypyridazin-4-yl]amino}pyridin-2-yl)-3-[4-(2,2,2-trifluoroethyl)piperazin-1-yl]propanamide;
N-(4-{[6-(5-chloro-2-fluorophenyl)-3-methoxypyridazin-4-yl]amino}pyridin-2-yl)-2-[4-(2,2,2-trifluoroethyl)piperazin-1-yl]acetamide;
methyl 2-{[6-(5-chloro-2-fluorophenyl)-4-({2-[3-(4-methylpiperazin-1-yl)propanamido]pyridin-4-yl}amino)pyridazin-3-yl]oxy}acetate;
N-(4-{[6-(5-chloro-2-fluorophenyl)-3-(methylsulfanyl)pyridazin-4-yl]amino}pyridin-2-yl)-3-(4-methylpiperazin-1-yl)propanamide;
N-(4-{[6-(5-chloro-2-fluorophenyl)-3-methanesulfinylpyridazin-4-yl]amino}pyridin-2-yl)-3-(4-methylpiperazin-1-yl)propanamide;
N-(4-{[6-(5-chloro-2-fluorophenyl)-3-methanesulfonylpyridazin-4-yl]amino}pyridin-2-yl)-3-(4-methylpiperazin-1-yl)propanamide;
N-(4-{[6-(5-chloro-2-fluorophenyl)-3-[imino(methyl)oxo-??-sulfanyl]pyridazin-4-yl]amino}pyridin-2-yl)-3-(4-methylpiperazin-1-yl)propanamide;
3-[4-(2-aminoethyl)piperazin-1-yl]-N-(4-{[6-(5-chloro-2-fluorophenyl)-3-(methylsulfanyl)pyridazin-4-yl]amino}pyridin-2-yl)propanamide;
methyl N-[2-(4-{2-[(4-{[6-(5-chloro-2-fluorophenyl)-3-(methylsulfanyl)pyridazin-4-yl]amino}pyridin-2-yl)carbamoyl]ethyl}piperazin-1-yl)ethyl]carbamate;
N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)propanamide;
N-(4-{[6-(5-chloro-2-fluorophenyl)-3-[(2-hydroxyethyl)sulfanyl]pyridazin-4-yl]amino}pyridin-2-yl)-4-(4-methylpiperazin-1-yl)butanamide;
N-(4-{[6-(5-chloro-2-fluorophenyl)-3-[(2-hydroxyethyl)sulfanyl]pyridazin-4-yl]amino}pyridin-2-yl)-2-{6-methyl-2,6-diazaspiro[3.3]heptan-2-yl}acetamide;
N-(4-{[6-(5-chloro-2-fluorophenyl)-3-[(2-hydroxyethyl)sulfanyl]pyridazin-4-yl]amino}pyridin-2-yl)-2-{5-methyl-2,5-diazabicyclo[2.2.1]heptan-2-yl}acetamide;
N-(4-{[6-(5-chloro-2-fluorophenyl)-3-[(2-hydroxyethyl)sulfanyl]pyridazin-4-yl]amino}pyridin-2-yl)-2-methyl-2,8-diazaspiro[4.5]decane-8-carboxamide;
N-(4-{[6-(5-chloro-2-fluorophenyl)-3-[(2-hydroxyethyl)sulfanyl]pyridazin-4-yl]amino}pyridin-2-yl)-2-(4-methyl-1,4-diazepan-1-yl)acetamide;
N-(4-{[6-(5-chloro-2-fluorophenyl)-3-[(3-hydroxypropyl)sulfanyl]pyridazin-4-yl]amino}pyridin-2-yl)-3-(4-methylpiperazin-1-yl)propanamide;
N-(4-{[6-(5-chloro-2-fluorophenyl)-3-(methylamino)pyridazin-4-yl]amino}pyridin-2-yl)-3-(4-methylpiperazin-1-yl)propanamide;
N-(4-{[6-(5-chloro-2-fluorophenyl)-3-(dimethylamino)pyridazin-4-yl]amino}pyridin-2-yl)-3-(4-methylpiperazin-1-yl)propanamide;
N-(4-{[6-(5-chloro-2-fluorophenyl)-3-(2-methoxyethoxy)pyridazin-4-yl]amino}pyridin-2-yl)-3-(4-methylpiperazin-1-yl)propanamide;
N-(4-{[6-(5-chloro-2-fluorophenyl)-3-(2-methoxyethoxy)pyridazin-4-yl]amino}pyridin-2-yl)-3-[4-(2,2,2-trifluoroethyl)piperazin-1-yl]propanamide;
N-(4-{[6-(5-chloro-2-fluorophenyl)-3-(2-methoxyethoxy)pyridazin-4-yl]amino}pyridin-2-yl)-3-(morpholin-4-yl)propanamide;
N-(4-{[6-(5-chloro-2-fluorophenyl)-3-[2-(4-methylpiperazin-1-yl)ethoxy]pyridazin-4-yl]amino}pyridin-2-yl)cyclopropanecarboxamide;
N-(4-{[6-(5-chloro-2-fluorophenyl)-3-[2-(dimethylamino)ethoxy]pyridazin-4-yl]amino}pyridin-2-yl)cyclopropanecarboxamide;
N-(4-{[6-(5-chloro-2-fluorophenyl)-3-[2-(dimethylamino)ethoxy]pyridazin-4-yl]amino}pyridin-2-yl)-3-(morpholin-4-yl)propanamide;
2-({4-[(2-aminopyridin-4-yl)amino]-6-(5-chloro-2-fluorophenyl)pyridazin-3-yl}oxy)ethan-1-ol;
N-(4-{[6-(5-chloro-2-fluorophenyl)-3-[(1-methylazetidin-3-yl)methoxy]pyridazin-4-yl]amino}pyridin-2-yl)cyclopropanecarboxamide;
N-[2-({4-[(2-aminopyridin-4-yl)amino]-6-(5-chloro-2-fluorophenyl)pyridazin-3-yl}oxy)ethyl]methanesulfonamide;
N-(4-{[6-(5-chloro-2-fluorophenyl)-3-methoxypyridazin-4-yl]amino}pyridin-2-yl)-3-(4-methylpiperazin-1-yl)propanamide;
N4-[6-(5-chloro-2-fluorophenyl)-3-methoxypyridazin-4-yl]pyridine-2,4-diamine;
N4-[6-(5-chloro-2-fluorophenyl)-3-(2,2,2-trifluoroethoxy)pyridazin-4-yl]pyridine-2,4-diamine;
N-(4-{[6-(5-chloro-2-fluorophenyl)-3-(2,2,2-trifluoroethoxy)pyridazin-4-yl]amino}pyridin-2-yl)-3-(4-methylpiperazin-1-yl)propanamide;
N-(4-{[6-(5-chloro-2-fluorophenyl)-3-(2,2-difluoroethoxy)pyridazin-4-yl]amino}pyridin-2-yl)-3-(4-methylpiperazin-1-yl)propanamide;
N4-[6-(5-chloro-2-fluorophenyl)-3-(2,2-difluoroethoxy)pyridazin-4-yl]pyridine-2,4-diamine;
N-(4-{[6-(5-chloro-2-fluorophenyl)-3-[2-(pyrrolidin-1-yl)ethoxy]pyridazin-4-yl]amino}pyridin-2-yl)cyclopropanecarboxamide;
N4-[6-(5-chloro-2-fluorophenyl)-3-[3-(methylsulfanyl)propoxy]pyridazin-4-yl]pyridine-2,4-diamine;
N4-[6-(5-chloro-2-fluorophenyl)-3-(3-methanesulfonylpropoxy)pyridazin-4-yl]pyridine-2,4-diamine;
N4-[6-(5-chloro-2-fluorophenyl)-3-(3-methanesulfinylpropoxy)pyridazin-4-yl]pyridine-2,4-diamine;
(3-{[6-(5-chloro-2-fluorophenyl)-4-[(2-cyclopropaneamidopyridin-4-yl)amino]pyridazin-3-yl]oxy}propyl)trimethylazanium chloride;
N-(4-{[6-(5-chloro-2-fluorophenyl)-3-[(2-hydroxyethyl)sulfanyl]pyridazin-4-yl]amino}pyridin-2-yl)-2-(piperazin-1-yl)acetamide;
N-(4-{[6-(5-chloro-2-fluorophenyl)-3-[(2-hydroxyethyl)sulfanyl]pyridazin-4-yl]amino}pyridin-2-yl)-2-(1,4-diazepan-1-yl)acetamide;
N-(4-{[6-(5-chloro-2-fluorophenyl)-3-(methylsulfanyl)pyridazin-4-yl]amino}pyridin-2-yl)-3-(piperazin-1-yl)propanamide;
N-(4-{[6-(3-fluoro-6-methylpyridin-2-yl)-3-(2,2,2-trifluoroethoxy)pyridazin-4-yl]amino}pyridin-2-yl)-3-(4-methylpiperazin-1-yl)propanamide;
N4-[6-(5-chloro-2-fluorophenyl)-3-(2-methoxyethoxy)pyridazin-4-yl]pyridine-2,4-diamine;
N4-[6-(5-chloro-2-fluorophenyl)-3-[2-(4-methylpiperazin-1-yl)ethoxy]pyridazin-4-yl]pyridine-2,4-diamine;
N-(4-{[6-(5-chloro-2-fluorophenyl)-3-{[(1s,3s)-3-hydroxycyclobutyl]methoxy}pyridazin-4-yl]amino}pyridin-2-yl)-3-(4-methylpiperazin-1-yl)propanamide;
N-(4-{[6-(5-chloro-2-fluorophenyl)-3-(oxolan-3-yloxy)pyridazin-4-yl]amino}pyridin-2-yl)-2-(4-methyl-1,4-diazepan-1-yl)acetamide;
N-(4-{[6-(5-chloro-2-fluorophenyl)-3-(oxolan-3-yloxy)pyridazin-4-yl]amino}pyridin-2-yl)-2-(4-methyl-1,4-diazepan-1-yl)acetamide;
N-(4-{[6-(5-chloro-2-fluorophenyl)-3-(oxolan-3-yloxy)pyridazin-4-yl]amino}pyridin-2-yl)-3-(4-methylpiperazin-1-yl)propanamide;
N-(4-{[6-(5-chloro-2-fluorophenyl)-3-[(2,2-dimethyl-1,3-dioxolan-4-yl)methoxy]pyridazin-4-yl]amino}pyridin-2-yl)-3-(4-methylpiperazin-1-yl)propanamide;
N-(4-{[6-(5-chloro-2-fluorophenyl)-3-(2,3-dihydroxypropoxy)pyridazin-4-yl]amino}pyridin-2-yl)-3-(4-methylpiperazin-1-yl)propanamide;
N-(4-{[6-(5-chloro-2-fluorophenyl)-3-[(2-oxo-1,3-dioxolan-4-yl)methoxy]pyridazin-4-yl]amino}pyridin-2-yl)-3-(4-methylpiperazin-1-yl)propanamide;
N-(4-{[6-(5-chloro-2-fluorophenyl)-3-[(1s,3s)-3-(hydroxymethyl)cyclobutoxy]pyridazin-4-yl]amino}pyridin-2-yl)-3-(4-methylpiperazin-1-yl)propanamide;
N-(4-{[6-(5-chloro-2-fluorophenyl)-3-[(3-hydroxyphenyl)methoxy]pyridazin-4-yl]amino}pyridin-2-yl)-3-(4-methylpiperazin-1-yl)propanamide;
N-(4-{[6-(5-chloro-2-fluorophenyl)-3-[(2-hydroxyethyl)sulfanyl]pyridazin-4-yl]amino}pyridin-2-yl)-2-{6-methyl-3,6-diazabicyclo[3.2.2]nonan-3-yl}acetamide;
Cis-N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)cyclobutane-1-carboxamide;
N-(4-{[6-(5-chloro-2-fluorophenyl)-3-[(2-hydroxyethyl)sulfanyl]pyridazin-4-yl]amino}pyridin-2-yl)-3-(4-methyl-1,4-diazepan-1-yl)propanamide;
N-(4-{[6-(5-chloro-2-fluorophenyl)-3-1{[(2,2-dimethyl-1,3-dioxolan-4-yl)methyl]sulfanyl}pyridazin-4-yl]amino}pyridin-2-yl)-3-(4-methylpiperazin-1-yl)propanamide;
N-(4-{[6-(5-chloro-2-fluorophenyl)-3-[(2,3-dihydroxypropyl)sulfanyl]pyridazin-4-yl]amino}pyridin-2-yl)-3-(4-methylpiperazin-1-yl)propanamide;
Cis-N-(4-{[6-(5-chloro-2-fluorophenyl)-3-[(2-hydroxyethyl)sulfanyl]pyridazin-4-yl]amino}pyridin-2-yl)-3-[(1S,4S)-5-methyl-2,5-diazabicyclo[2.2.1]heptan-2-yl]cyclobutane-1-carboxamide;
Trans-N-(4-{[6-(5-chloro-2-fluorophenyl)-3-[(2-hydroxyethyl)sulfanyl]pyridazin-4-yl]amino}pyridin-2-yl)-3-[(1S,4S)-5-methyl-2,5-diazabicyclo[2.2.1]heptan-2-yl]cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-(thiomorpholin-4-yl)cyclobutane-1-carboxamide;
Cis N-(4-{[6-(5-chloro-2-fluorophenyl)-3-[(2-hydroxyethyl)sulfanyl] pyridazin-4-yl]amino}pyridin-2-yl)-3-{4-methyl-4,7-diazaspiro[2.5]octan-7-yl}cyclobutane-1-carboxamide;
methyl 5-[(4-{[6-(5-chloro-2-fluorophenyl)-3-[(2-hydroxyethyl)sulfanyl] pyridazin-4-yl]amino}pyridin-2-yl)amino]-3-(1-methylpiperidin-4-yl)thiophene-2-carboxylate;
N-(4-{[6-(5-chloro-2-fluorophenyl)-3-[(1-hydroxy-2-methylpropan-2-yl)sulfanyl]pyridazin-4-yl]amino}pyridin-2-yl)-3-(4-methylpiperazin-1-yl)propanamide;
(1s,3s)-N-(4-{[6-(5-chloro-2-fluorophenyl)-3-[(1-hydroxy-2-methylpropan-2-yl)sulfanyl]pyridazin-4-yl]amino}pyridin-2-yl)-3-(4-methylpiperazin-1-yl)cyclobutane-1-carboxamide;
N-(4-{[6-(5-chloro-2-fluorophenyl)-3-[3-(hydroxymethyl)azetidin-1-yl]pyridazin-4-yl]amino}pyridin-2-yl)-3-(4-methylpiperazin-1-yl)propanamide;
Trans-N-(4-{[6-(5-chloro-2-fluorophenyl)-3-(dimethylamino)pyridazin-4-yl]amino}pyridin-2-yl)-3-(4-methylpiperazin-1-yl)cyclobutane-1-carboxamide;
Cis-N-(4-{[6-(5-chloro-2-fluorophenyl)-3-(dimethylamino)pyridazin-4-yl]amino}pyridin-2-yl)-3-(4-methylpiperazin-1-yl)cyclobutane-1-carboxamide;
methyl 1-[6-(5-chloro-2-fluorophenyl)-4-({2-[3-(4-methylpiperazin-1-yl)propanamido]pyridin-4-yl}amino)pyridazin-3-yl]azetidine-3-carboxylate;
1-[6-(5-chloro-2-fluorophenyl)-4-({2-[3-(4-methylpiperazin-1-yl)propanamido]pyridin-4-yl}amino)pyridazin-3-yl]azetidine-3-carboxylic acid;
propan-2-yl 1-[6-(5-chloro-2-fluorophenyl)-4-({2-[3-(4-methylpiperazin-1-yl)propanamido]pyridin-4-yl}amino)pyridazin-3-yl]azetidine-3-carboxylate;
N-(4-{[6-(5-chloro-2-fluorophenyl)-3-1{[(3-hydroxyphenyl)methyl]amino}pyridazin-4-yl]amino}pyridin-2-yl)-3-(4-methylpiperazin-1-yl)propanamide;
N-(4-{[6-(5-chloro-2-fluorophenyl)-3-{[(3-hydroxyphenyl)methyl](methyl)amino}pyridazin-4-yl]amino}pyridin-2-yl)-3-(4-methylpiperazin-1-yl)propanamide;
N-(4-{[6-(5-chloro-2-fluorophenyl)-3-(dimethylamino)pyridazin-4-yl]amino}pyridin-2-yl)-2-(4-methyl-1,4-diazepan-1-yl)acetamide;
N-(4-{[6-(5-chloro-2-fluorophenyl)-3-{7-oxo-6-oxa-2-azaspiro[3.4]octan-2-yl}pyridazin-4-yl]amino}pyridin-2-yl)-3-(4-methylpiperazin-1-yl)propanamide;
N-(4-{[6-(5-chloro-2-fluorophenyl)-3-[methyl(oxolan-3-yl)amino]pyridazin-4-yl]amino}pyridin-2-yl)-3-(4-methylpiperazin-1-yl)propanamide;
N-(4-{[6-(5-chloro-2-fluorophenyl)-3-[methyl(oxolan-3-yl)amino]pyridazin-4-yl]amino}pyridin-2-yl)-3-(4-methylpiperazin-1-yl)propanamide;
N-(4-{[6-(5-chloro-2-fluorophenyl)-3-{methyl[(2-oxooxolan-3-yl)methyl]amino}pyridazin-4-yl]amino}pyridin-2-yl)-3-(4-methylpiperazin-1-yl)propanamide;
methyl 1-[6-(5-chloro-2-fluorophenyl)-4-[(2-{2-[(1S,4S)-5-methyl-2,5-diazabicyclo[2.2.1]heptan-2-yl]acetamido}pyridin-4-yl)amino]pyridazin-3-yl]azetidine-3-carboxylate;
N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)propanamide;
Trans N-(4-{[6-(5-chloro-2-fluorophenyl)-3-(oxolan-3-yloxy)pyridazin-4-yl]amino}pyridin-2-yl)-3-(4-methylpiperazin-1-yl)cyclobutane-1-carboxamide;
Cis N-(4-{[6-(5-chloro-2-fluorophenyl)-3-(oxolan-3-yloxy)pyridazin-4-yl]amino}pyridin-2-yl)-3-(4-methylpiperazin-1-yl)cyclobutane-1-carboxamide;
Cis N-(4-{[6-(5-chloro-2-fluorophenyl)-3-{[(2,2-dimethyl-1,3-dioxolan-4-yl)methyl]sulfanyl}pyridazin-4-yl]amino}pyridin-2-yl)-3-(4-methylpiperazin-1-yl)cyclobutane-1-carboxamide;
N-(4-{[6-(5-chloro-2-fluorophenyl)-3-[(2,2-dimethyl-2H-1,3-benzodioxol-5-yl)methoxy]pyridazin-4-yl]amino}pyridin-2-yl)-3-(4-methylpiperazin-1-yl)propanamide;
Trans N-(4-{[6-(5-chloro-2-fluorophenyl)-3-[(3-hydroxycyclobutyl) methoxy]pyridazin-4-yl]amino}pyridin-2-yl)-3-(4-methylpiperazin-1-yl)cyclobutane-1-carboxamide;
Cis N-(4-{[6-(5-chloro-2-fluorophenyl)-3-[(3-hydroxycyclobutyl) methoxy]pyridazin-4-yl]amino}pyridin-2-yl)-3-(4-methylpiperazin-1-yl)cyclobutane-1-carboxamide;
Cis N-(4-{[6-(5-chloro-2-fluorophenyl)-3-[(3-hydroxyphenyl) methoxy]pyridazin-4-yl]amino}pyridin-2-yl)-3-(4-methylpiperazin-1-yl)cyclobutane-1-carboxamide;
N-(4-{[6-(5-chloro-2-fluorophenyl)-3-[(3-hydroxyphenyl)methoxy] pyridazin-4-yl]amino}pyridin-2-yl)-2-[(1S,4S)-5-methyl-2,5-diazabicyclo[2.2.1]heptan-2-yl]acetamide;
N-(4-{[6-(5-chloro-2-fluorophenyl)-3-[(3-hydroxycyclobutyl)methoxy] pyridazin-4-yl]amino}pyridin-2-yl)-2-(4-methyl-1,4-diazepan-1-yl)acetamide;
N-(4-{[6-(5-chloro-2-fluorophenyl)-3-[(3-hydroxycyclobutyl)methoxy] pyridazin-4-yl]amino}pyridin-2-yl)-2-[(1S,4S)-5-methyl-2,5-diazabicyclo[2.2.1]heptan-2-yl]acetamide;
N-(4-{[6-(5-chloro-2-fluorophenyl)-3-[(3-hydroxy-3-methylcyclobutyl)methoxy]pyridazin-4-yl]amino}pyridin-2-yl)-3-(4-methylpiperazin-1-yl)propanamide;
Cis N-(4-{[6-(5-chloro-2-fluorophenyl)-3-[(3-hydroxy-3-methylcyclobutyl) methoxy]pyridazin-4-yl]amino}pyridin-2-yl)-3-(4-methylpiperazin-1-yl)cyclobutane-1-carboxamide;
methyl 3-({[6-(5-chloro-2-fluorophenyl)-4-({2-[3-(4-methylpiperazin-1-yl)propanamido]pyridin-4-yl}amino)pyridazin-3-yl]oxy}methyl)bicyclo[1.1.1]pentane-1-carboxylate;
Cis methyl 3-({[6-(5-chloro-2-fluorophenyl)-4-({2-[-3-(4-methylpiperazin-1-yl)cyclobutaneamido]pyridin-4-yl}amino)pyridazin-3-yl]oxy}methyl)bicyclo[1.1.1]pentane-1-carboxylate;
N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)propanamide;
N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)propanamide;
N-(4-{[6-(5-chloro-2-fluorophenyl)-3-[methyl(4,4,4-trifluoro-3-hydroxybutyl)amino]pyridazin-4-yl]amino}pyridin-2-yl)-2-[(1S,4S)-5-methyl-2,5-diazabicyclo[2.2.1]heptan-2-yl]acetamide;
N-(4-{[6-(5-chloro-2-fluorophenyl)-3-[methyl(4,4,4-trifluoro-3-hydroxybutyl)amino]pyridazin-4-yl]amino}pyridin-2-yl)-2-[(1S,4S)-5-methyl-2,5-diazabicyclo[2.2.1]heptan-2-yl]acetamide;
methyl 4-[6-(5-chloro-2-fluorophenyl)-4-({2-[3-(4-methylpiperazin-1-yl)propanamido]pyridin-4-yl}amino)pyridazin-3-yl]morpholine-2-carboxylate
4-[6-(5-chloro-2-fluorophenyl)-4-({2-[3-(4-methylpiperazin-1-yl)propanamido]pyridin-4-yl}amino)pyridazin-3-yl]morpholine-2-carboxylate lithium salt;
Cis N-(4-{[6-(5-chloro-2-fluorophenyl)-3-{methyl[(3-methyl-2-oxooxolan-3-yl)methyl]amino}pyridazin-4-yl]amino}pyridin-2-yl)-3-(4-methylpiperazin-1-yl)cyclobutane-1-carboxamide;
N-(4-((6-(5-chloro-2-fluorophenyl)-3-(methyl((3-methyl-2-oxooxolan-3-yl)methyl)amino)pyridazin-4-yl)amino)pyridin-2-yl)-2-(4-methyl-1,4-diazepan-1-yl)acetamide;
N-(4-((6-(5-chloro-2-fluorophenyl)-3-(methyl((3-methyl-2-oxooxolan-3-yl)methyl)amino)pyridazin-4-yl)amino)pyridin-2-yl)-2-(4-methyl-1,4-diazepan-1-yl)acetamide;
ethyl 3-{[6-(5-chloro-2-fluorophenyl)-4-({2-[3-(4-methylpiperazin-1-yl)propanamido]pyridin-4-yl}amino)pyridazin-3-yl](methyl)amino}-2,2-dimethylpropanoate;
Cis N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)cyclobutane-1-carboxamide;
Trans N-(4-{[6-(5-chloro-2-fluorophenyl)-3-[methyl(4,4,4-trifluoro-3-hydroxybutyl)amino]pyridazin-4-yl]amino}pyridin-2-yl)-3-(4-methylpiperazin-1-yl)cyclobutane-1-carboxamide;
propan-2-yl 1-[6-(5-chloro-2-fluorophenyl)-4-({2-[3-(4-methylpiperazin-1-yl)propanamido]pyridin-4-yl}amino)pyridazin-3-yl]azetidine-2-carboxylate;
ammonium 1-[6-(5-chloro-2-fluorophenyl)-4-({2-[3-(4-methylpiperazin-1-yl)propanamido]pyridin-4-yl}amino)pyridazin-3-yl]azetidine-2-carboxylate;
N-(4-{[6-(5-chloro-2-fluorophenyl)-3-({[3-(hydroxymethyl)-2-oxooxolan-3-yl]methyl}(methyl)amino)pyridazin-4-yl]amino}pyridin-2-yl)-3-(4-methylpiperazin-1-yl)propanamide;
N-(4-{[6-(5-chloro-2-fluorophenyl)-3-[(2-hydroxyethyl)sulfanyl]pyridazin-4-yl]amino}pyridin-2-yl)-3-(piperazin-1-yl)propanamide;
N-(4-{[6-(5-chloro-2-fluorophenyl)-3-{[(3-hydroxycyclobutyl)methyl]sulfanyl}pyridazin-4-yl]amino}pyridin-2-yl)-3-(4-methylpiperazin-1-yl)propanamide;
Cis N-(4-{[6-(5-chloro-2-fluorophenyl)-3-[(2-hydroxyethyl)sulfanyl]pyridazin-4-yl]amino}pyridin-2-yl)-3-(4-methyl-1,4-diazepan-1-yl)cyclobutane-1-carboxamide;
Trans N-(4-{[6-(5-chloro-2-fluorophenyl)-3-[(2-hydroxyethyl)sulfanyl]pyridazin-4-yl]amino}pyridin-2-yl)-3-[4-(propan-2-yl)piperazin-1-yl]cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-[4-(propan-2-yl)piperazin-1-yl]cyclobutane-1-carboxamide;
Trans N-(4-{[6-(5-chloro-2-fluorophenyl)-3-[(2-hydroxyethyl)sulfanyl]pyridazin-4-yl]amino}pyridin-2-yl)-3-(4-ethylpiperazin-1-yl)cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-(4-ethylpiperazin-1-yl)cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-(4-cyclopropylpiperazin-1-yl)cyclobutane-1-carboxamide;
Trans N-(4-{[6-(5-chloro-2-fluorophenyl)-3-[(2-hydroxyethyl)sulfanyl]pyridazin-4-yl]amino}pyridin-2-yl)-3-[4-fluoro-4-(hydroxymethyl)piperidin-1-yl]cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-[4-fluoro-4-(hydroxymethyl)piperidin-1-yl]cyclobutane-1-carboxamide;
Trans N-(4-{[6-(5-chloro-2-fluorophenyl)-3-[(2-hydroxyethyl)sulfanyl]pyridazin-4-yl]amino}pyridin-2-yl)-3-(4-methoxypiperidin-1-yl)cyclobutane-1-carboxamide;
Cis N-(4-{[6-(5-chloro-2-fluorophenyl)-3-[(2-hydroxyethyl)sulfanyl] pyridazin-4-yl]amino}pyridin-2-yl)-3-(4-methoxypiperidin-1-yl)cyclobutane-1-carboxamide;
Trans ethyl 1-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}piperidine-4-carboxylate;
Cis ethyl 1-{3-[(4-{[6-(5-chloro-2-fluorophenyl)-3-[(2-hydroxyethyl)sulfanyl]pyridazin-4-yl]amino}pyridin-2-yl)carbamoyl]cyclobutyl}piperidine-4-carboxylate;
Cis 1-{3-[(4-{[6-(5-chloro-2-fluorophenyl)-3-[(2-hydroxyethyl)sulfanyl] pyridazin-4-yl]amino}pyridin-2-yl)carbamoyl]cyclobutyl}piperidine-4-carboxylic acid;
Trans N-(4-{[6-(5-chloro-2-fluorophenyl)-3-[(2-hydroxyethyl)sulfanyl] pyridazin-4-yl]amino}pyridin-2-yl)-3-(4-methylpiperidin-1-yl)cyclobutane-1-carboxamide;
Cis N-(4-{[6-(5-chloro-2-fluorophenyl)-3-[(2-hydroxyethyl)sulfanyl] pyridazin-4-yl]amino}pyridin-2-yl)-3-(4-methylpiperidin-1-yl)cyclobutane-1-carboxamide;
Trans N-(4-{[6-(5-chloro-2-fluorophenyl)-3-[(2-hydroxyethyl)sulfanyl] pyridazin-4-yl]amino}pyridin-2-yl)-3-[4,4-difluoro-3-(hydroxymethyl)piperidin-1-yl]cyclobutane-1-carboxamide;
Cis N-(4-{[6-(5-chloro-2-fluorophenyl)-3-[(2-hydroxyethyl)sulfanyl] pyridazin-4-yl]amino}pyridin-2-yl)-3-[4,4-difluoro-3-(hydroxymethyl)piperidin-1-yl]cyclobutane-1-carboxamide;
Cis N-(4-{[6-(5-chloro-2-fluorophenyl)-3-[(2-hydroxyethyl)sulfanyl] pyridazin-4-yl]amino}pyridin-2-yl)-3-[3-(2-fluoroethyl)-4-methylpiperazin-1-yl]cyclobutane-1-carboxamide;
Trans N-(4-{[6-(5-chloro-2-fluorophenyl)-3-[(2-hydroxyethyl)sulfanyl] pyridazin-4-yl]amino}pyridin-2-yl)-3-{5-methyl-5,8-diazaspiro[3.5]nonan-8-yl}cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-{5-methyl-5,8-diazaspiro[3.5]nonan-8-yl}cyclobutane-1-carboxamide;
Cis N-(4-{[6-(5-chloro-2-fluorophenyl)-3-[(2-hydroxyethyl)sulfanyl]pyridazin-4-yl]amino}pyridin-2-yl)-3-{6-methyl-3,6-diazabicyclo[3.1.1]heptan-3-yl}cyclobutane-1-carboxamide;
N-(4-{[6-(5-chloro-2-fluorophenyl)-3-[(2-hydroxyethyl)sulfanyl]pyridazin-4-yl]amino}pyridin-2-yl)-3-(3,5-dimethylpiperazin-1-yl)propanamide;
N-(4-{[6-(5-chloro-2-fluorophenyl)-3-sulfanylpyridazin-4-yl]amino}pyridin-2-yl)-3-(4-methylpiperazin-1-yl)propanamide;
N-(4-{[6-(5-chloro-2-fluorophenyl)-3-[(2-hydroxyethyl)sulfanyl]pyridazin-4-yl]amino}pyridin-2-yl)-3-[(4-methylpiperazin-1-yl)methyl]bicyclo[1.1.1]pentane-1-carboxamide;
N-(4-{[6-(5-chloro-2-fluorophenyl)-3-[(2-hydroxyethyl)sulfanyl]pyridazin-4-yl]amino}pyridin-2-yl)-3-[(4-cyclopropylpiperazin-1-yl)methyl]bicyclo[1.1.1]pentane-1-carboxamide;
propan-2-yl 1-[6-(5-chloro-2-fluorophenyl)-4-({2-[3-(3,5-dimethylpiperazin-1-yl)propanamido]pyridin-4-yl}amino)pyridazin-3-yl]azetidine-2-carboxylate;
Cis N-(4-{[6-(5-chloro-2-fluorophenyl)-3-[(2-hydroxyethyl)sulfanyl]pyridazin-4-yl]amino}pyridin-2-yl)-3-(4-methylpiperazin-1-yl)cyclopentane-1-carboxamide;
N-(4-{[6-(5-chloro-2-fluorophenyl)-3-1{[(5-methyl-2-oxo-2H-1,3-dioxol-4-yl)methyl]sulfanyl}pyridazin-4-yl]amino}pyridin-2-yl)-3-(4-methylpiperazin-1-yl)propanamide;
N-(4-{[6-(5-chloro-2-fluorophenyl)-3-[(2-hydroxyethyl)sulfanyl]pyridazin-4-yl]amino}pyridin-2-yl)-3-[(3,5-dimethylpiperazin-1-yl)methyl]bicyclo[1.1.1]pentane-1-carboxamide;
N-(4-{[6-(5-chloro-2-fluorophenyl)-3-[(3-methyl-2-oxooxolan-3-yl)sulfanyl]pyridazin-4-yl]amino}pyridin-2-yl)-3-(4-methylpiperazin-1-yl)propanamide;
N-(4-{[6-(5-chloro-2-fluorophenyl)-3-[(2-hydroxyethyl)sulfanyl]pyridazin-4-yl]amino}pyridin-2-yl)-2-(3,5-dimethylpiperazin-1-yl)acetamide;
N-(4-{[6-(5-chloro-2-fluorophenyl)-3-{[2-(2-hydroxyethoxy)ethyl]sulfanyl}pyridazin-4-yl]amino}pyridin-2-yl)-3-(4-methylpiperazin-1-yl)propanamide;
3-({[6-(5-chloro-2-fluorophenyl)-4-({2-[3-(4-methylpiperazin-1-yl)propanamido]pyridin-4-yl}amino)pyridazin-3-yl]sulfanyl}methyl)benzoic acid;
N-(4-{[6-(5-chloro-2-fluorophenyl)-3-{1[(3-methyl-2-oxooxolan-3-yl)methyl]sulfanyl}pyridazin-4-yl]amino}pyridin-2-yl)-3-(4-methylpiperazin-1-yl)propanamide;
N-(4-{[6-(5-chloro-2-fluorophenyl)-3-({[3-(methoxymethyl)-2-oxooxolan-3-yl]methyl}(methyl)amino)pyridazin-4-yl]amino}pyridin-2-yl)-3-(4-methylpiperazin-1-yl)propanamide;
N-(4-{[6-(5-chloro-2-fluorophenyl)-3-({[3-(methoxymethyl)-2-oxooxolan-3-yl]methyl}(methyl)amino)pyridazin-4-yl]amino}pyridin-2-yl)-3-(4-methylpiperazin-1-yl)propanamide;
N-(4-{[6-(5-chloro-2-fluorophenyl)-3-({[3-(methoxymethyl)-2-oxooxolan-3-yl]methyl}(methyl)amino)pyridazin-4-yl]amino}pyridin-2-yl)-3-(4-methylpiperazin-1-yl)propanamide;
N-(4-{[6-(5-chloro-2-fluorophenyl)-3-{[(6-oxooxan-2-yl)methyl]sulfanyl}pyridazin-4-yl]amino}pyridin-2-yl)-3-(4-methylpiperazin-1-yl)propanamide; and
N-(2-{[6-(5-chloro-2-fluorophenyl)-4-({2-[3-(4-methylpiperazin-1-yl)propanamido]pyridin-4-yl}amino)pyridazin-3-yl]sulfanyl}ethyl)-5-oxooxolane-3-carboxamide.

4. The compound or salt according to claim 1, wherein A is a group A2 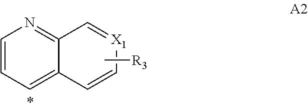
according to formula (Ib); 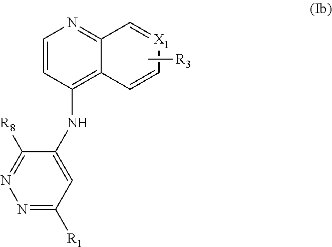
wherein:
X₁ is C or CH;
R₃ is —OR₇;
R₇ is selected from the group consisting of —(C₁-C₆)alkyl and —(C₁-C₆)alkylene-(C₃-C₉)heterocycloalkyl, wherein the —(C₃-C₉)heterocycloalkyl is optionally substituted by one or more —(C₁-C₆)alkyl;
R₈ is selected from the group consisting of —NR_{A}R_{B}, —O—(C₁-C₆)alkyl, —O—(C₁-C₆)haloalkyl, —O—(C₁-C₆)alkylene-OH, wherein the —O—(C₁-C₆)alkylene is substituted by one or more selected from the group consisting of —OH, —O—(C₁-C₆)alkylene-C(O)O—(C₁-C₆)alkyl, —O—(C₁-C₆)alkylene-NR_{A}R_{B}, —O—(C₁-C₆)alkylene-N⁺R_{A}R_{B}R_{C}, —O—(C₁-C₆)alkylene-S—(C₁-C₆)alkyl, —O—(C₁-C₆)alkylene-S(O)—(C₁-C₆)alkyl, —O—(C₁-C₆)alkylene-S(O)₂—(C₁-C₆)alkyl, —O—(C₁-C₆)alkylene-NH—S(O)₂—(C₁-C₆)alkyl, —O—(C₁-C₆)alkylene-O—(C₁-C₆)alkyl and —O—(C₁-C₆)alkylene-(C₃-C₉)heterocycloalkyl, wherein the —(C₃-C₉)heterocycloalkyl is optionally substituted by one or more —(C₁-C₆)alkyl;
R_{A} is H or —(C₁-C₆)alkyl;
R_{B} is H or selected from the group consisting of —(C₁-C₆)alkyl- and —S(O)₂—(C₁-C₆)alkyl; and
R_{C} is —(C₁-C₆)alkyl.

5. The compound or salt according to claim 4, wherein A is a group A2a 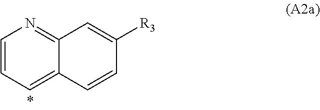
according to formula (Iba); 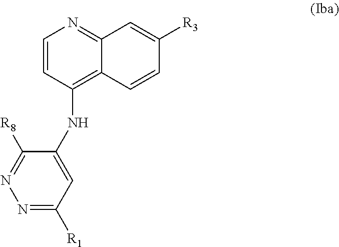
wherein:
R₃ is —OR₇;
R₇ is selected from the group consisting of —(C₁-C₆)alkyl and —(C₁-C₆)alkylene-(C₃-C₉)heterocycloalkyl, wherein the —(C₃-C₉)heterocycloalkyl is optionally substituted by one or more —(C₁-C₆)alkyl;
R₈ is selected from the group consisting of —NR_{A}R_{B}, —S—(C₁-C₆)alkyl, —S—(C₁-C₆)alkylene-OH, —S(O)=NH—(C₁-C₆)alkyl, —S(O)₂—(C₁-C₆)alkyl, —S(O)—(C₁-C₆)alkyl, —O—(C₁-C₆)alkyl, —O—(C₁-C₆)haloalkyl, —O—(C₁-C₆)alkylene-OH, wherein the —O—(C₁-C₆)alkylene is substituted by one or more selected from the group consisting of —OH, —O—(C₁-C₆)alkylene-C(O)O—(C₁-C₆)alkyl, —O—(C₁-C₆)alkylene-NR_{A}R_{B}, —O—(C₁-C₆)alkylene-N⁺R_{A}R_{B}R_{C}, —O—(C₁-C₆)alkylene-S—(C₁-C₆)alkyl, —O—(C₁-C₆)alkylene-S(O)—(C₁-C₆)alkyl, —O—(C₁-C₆)alkylene-S(O)₂—(C₁-C₆)alkyl, —O—(C₁-C₆)alkylene-NH—S(O)₂—(C₁-C₆)alkyl, —O—(C₁-C₆)alkylene-O—(C₁-C₆)alkyl and —O—(C₁-C₆)alkylene-(C₃-C₉)heterocycloalkyl, wherein the —(C₃-C₉)heterocycloalkyl is optionally substituted by one or more —(C₁-C₆)alkyl;
R_{A} is H or —(C₁-C₆)alkyl;
R_{B} is H or selected from the group consisting of —(C₁-C₆)alkyl and —S(O)₂—(C₁-C₆)alkyl; and
R_{C} is —(C₁-C₆)alkyl.

6. The compound or salt according to claim 5, wherein the compound is selected from the group consisting of:
2-{[6-(5-chloro-2-fluorophenyl)-4-({7-[2-(4-methylpiperazin-1-yl)ethoxy]quinolin-4-yl}amino)pyridazin-3-yl]oxy}ethan-1-ol;
N-[6-(5-chloro-2-fluorophenyl)-3-(2,2-difluoroethoxy)pyridazin-4-yl]-7-[2-(4-methylpiperazin-1-yl)ethoxy]quinolin-4-amine;
N-[3-(2-aminoethoxy)-6-(5-chloro-2-fluorophenyl)pyridazin-4-yl]-7-[2-(4-methylpiperazin-1-yl)ethoxy]quinolin-4-amine;
N-(2-{[6-(5-chloro-2-fluorophenyl)-4-({7-[2-(4-methylpiperazin-1-yl)ethoxy]quinolin-4-yl}amino)pyridazin-3-yl]oxy}ethyl)methanesulfonamide;
N-[6-(5-chloro-2-fluorophenyl)-3-(2-methoxyethoxy)pyridazin-4-yl]-7-[2-(4-methylpiperazin-1-yl)ethoxy]quinolin-4-amine;
N-[6-(5-chloro-2-fluorophenyl)-3-[2-(4-methylpiperazin-1-yl)ethoxy]pyridazin-4-yl]-7-methoxyquinolin-4-amine;
N-[6-(5-chloro-2-fluorophenyl)-3-[2-(dimethylamino)ethoxy]pyridazin-4-yl]-7-methoxyquinolin-4-amine;
N-[6-(5-chloro-2-fluorophenyl)-3-methoxypyridazin-4-yl]-7-[2-(4-methylpiperazin-1-yl)ethoxy]quinolin-4-amine; and
N-[6-(5-chloro-2-fluorophenyl)-3-(2,2,2-trifluoroethoxy)pyridazin-4-yl]-7-[2-(4-methylpiperazin-1-yl)ethoxy]quinolin-4-amine.

7. The compound or salt according to claim 1, wherein A is a group A3 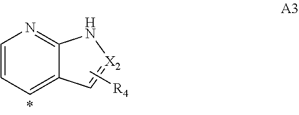
the according to formula (Ic) 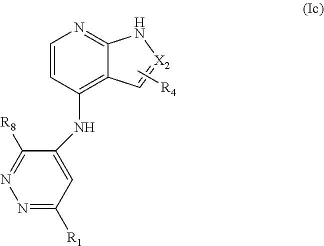
wherein:
R₁ is selected from the group consisting of aryl and pyridyl, wherein the aryl and pyridyl are optionally substituted by one or more halogen atoms;
X₂ is C, CH or N;
R₄ is H or —C(O)O—(C₁-C₆)alkyl;
R₈ is selected from the group consisting of:
—NR_{A}R_{B};
—S—(C₁-C₆)alkylene-aryl, wherein the aryl is optionally substituted by one or more groups selected from the group consisting of —C(O)O—(C₁-C₆)alkylene-NR_{A}R_{C} and —C(O)O—(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo;
—O—(C₁-C₆)alkyl;
—O—(C₁-C₆)haloalkyl;
—O—(C₁-C₆)alkylene-OH, wherein the —O—(C₁-C₆)alkylene is substituted by one or more —OH;
—O—(C₁-C₆)alkylene-C(O)O—(C₁-C₆)alkyl;
—O—(C₁-C₆)alkylene-NR_{A}R_{B};
—O—(C₁-C₆)alkylene-N⁺R_{A}R_{B}R_{C};
—O—(C₁-C₆)alkylene-S—(C₁-C₆)alkyl;
—O—(C₁-C₆)alkylene-S(O)—(C₁-C₆)alkyl;
—O—(C₁-C₆)alkylene-S(O)₂—(C₁-C₆)alkyl;
—O—(C₁-C₆)alkylene-O—(C₁-C₆)alkyl; and
—O—(C₁-C₆)alkylene-(C₃-C₉)heterocycloalkyl, wherein the —(C₃-C₉)heterocycloalkyl is optionally substituted by one or more —(C₁-C₆)alkyl;
R_{A} is H or —(C₁-C₆)alkyl;
R_{B} is H or selected from the group consisting of —(C₁-C₆)alkyl- and —S(O)₂—(C₁-C₆)alkyl; and
R_{C} is —(C₁-C₆)alkyl.

8. The compound or salt according to claim 7, wherein the compound is selected from the group consisting of:
methyl 4-{[6-(5-chloro-2-fluorophenyl)-3-[3-(dimethylamino)propoxy] pyridazin-4-yl]amino}-1H-pyrrolo[2,3-b]pyridine-2-carboxylate;
(3-{[6-(5-chloro-2-fluorophenyl)-4-{[2-(methoxycarbonyl)-1H-pyrrolo[2,3-b]pyridin-4-yl]amino}pyridazin-3-yl]oxy}propyl)trimethylazanium chloride;
6-(5-chloro-2-fluorophenyl)-3-[2-(4-methylpiperazin-1-yl)ethoxy]-N-{1H-pyrrolo[2,3-b]pyridin-4-yl}pyridazin-4-amine;
6-(5-chloro-2-fluorophenyl)-3-[2-(dimethylamino)ethoxy]-N-{1H-pyrrolo[2,3-b]pyridin-4-yl}pyridazin-4-amine;
6-(5-chloro-2-fluorophenyl)-3-[2-(dimethylamino)ethoxy]-N-{1H-pyrazolo[3,4-b]pyridin-4-yl}pyridazin-4-amine;
methylpiperidin-4-yl)methyl 3-({[6-(5-chloro-2-fluorophenyl)-4-({1H-pyrrolo[2,3-b]pyridin-4-yl}amino)pyridazin-3-yl]sulfanyl}methyl)benzoate; and
2-(dimethylamino)ethyl 3-({[6-(5-chloro-2-fluorophenyl)-4-({1H-pyrrolo[2,3-b]pyridin-4-yl}amino)pyridazin-3-yl]sulfanyl}methyl)benzoate.

9. The compound or salt according to claim 1, wherein A is a group A4 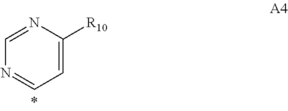
according to formula (Id); 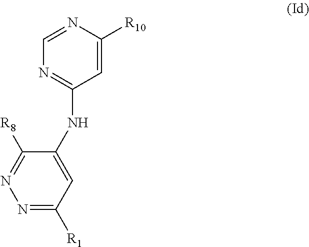
wherein:
R₁ is aryl optionally substituted by one or more halogen atoms;
R₁₀ is —NR₅C(O)R₆;
R₅ is H;
R₆ is selected from the group consisting of:
—(C₃-C₆)cycloalkyl substituted by one or more —(C₃-C₉)heterocycloalkyl, wherein the —(C₃-C₉)heterocycloalkyl is optionally substituted by one or more groups selected from the group consisting of —(C₁-C₆)alkyl and —(C₃-C₆)cycloalkyl;
—(C₁-C₆)alkylene-(C₃-C₉)heterocycloalkyl, wherein the —(C₃-C₉)heterocycloalkyl is optionally substituted by one or more —(C₁-C₆)alkyl; and
—(C₃-C₆)cycloalkyl optionally substituted by one or more —(C₁-C₆)alkylene-(C₃-C₉)heterocycloalkyl, wherein the —(C₃-C₉)heterocycloalkyl is optionally substituted by one or more —(C₁-C₆)alkyl;
R₈ is selected from the group consisting of:
—NR_{A}R_{B};
—S—(C₁-C₆)alkyl, wherein the —(C₁-C₆)alkyl is optionally substituted by one or more —OH;
—S—(C₁-C₆)alkylene-OH, wherein the —(C₁-C₆)alkylene is optionally substituted by one or more —(C₁-C₆)alkyl;
—S—(C₁-C₆)alkylene-(C₃-C₉)heterocycloalkyl, wherein the —(C₃-C₉)heterocycloalkyl is optionally substituted by one or more —(C₁-C₆)alkyl;
—S(O)=NH—(C₁-C₆)alkyl;
—S(O)₂—(C₁-C₆)alkyl;
—S(O)—(C₁-C₆)alkyl; and
—S—(C₁-C₆)alkylene-Si((C₁-C₆)alkyl)₃;
R_{A} is H or —(C₁-C₆)alkyl; and
R_{B} is —(C₁-C₆)alkylene-(C₃-C₉)heterocycloalkyl, wherein the —(C₃-C₉)heterocycloalkyl is optionally substituted by one or more groups selected from the group consisting of —(C₁-C₆)alkyl and oxo; or alternatively
R_{A} and R_{B} together with the nitrogen atom to which they are attached may form a —(C₃-C₆)heterocycloalkyl, wherein the —(C₃-C₆)heterocycloalkyl is optionally substituted by one or more groups selected from the group consisting of —C(O)OH, —(C₁-C₆)alkylene-OH, —C(O)O—(C₁-C₆)alkyl and oxo, or the —(C₃-C₆)heterocycloalkyl is optionally substituted on two adjacent carbon atoms forming an additional condensed —(C₅-C₆)heterocycloalkyl, optionally substituted by oxo.

10. The compound or salt according to claim 9, wherein the compound is selected from the group consisting of:
Cis N-(6-{[6-(5-chloro-2-fluorophenyl)-3-[(2-hydroxyethyl)sulfanyl]pyridazin-4-yl]amino}pyrimidin-4-yl)-3-(4-methylpiperazin-1-yl)cyclobutane-1-carboxamide;
N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)propanamide;
Enantiomer 1 N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)propanamide;
Enantiomer 2 N-(6-{[6-(5-chloro-2-fluorophenyl)-3-1{methyl[(3-methyl-2-oxooxolan-3-yl)methyl]amino}pyridazin-4-yl]amino}pyrimidin-4-yl)-3-(4-methylpiperazin-1-yl)propanamide;
N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide;
N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide;
Cis N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(4-methylpiperazin-1-yl)cyclobutane-1-carboxamide;
N-(6-{[6-(5-chloro-2-fluorophenyl)-3-[(2-hydroxyethyl)sulfanyl]pyridazin-4-yl]amino}pyrimidin-4-yl)-3-(4-methylpiperazin-1-yl)propanamide;
N-(6-{[6-(5-chloro-2-fluorophenyl)-3-[(2-hydroxyethyl)sulfanyl]pyridazin-4-yl]amino}pyrimidin-4-yl)-3-(3,5-dimethylpiperazin-1-yl)propanamide;
Cis N-(6-{[6-(5-chloro-2-fluorophenyl)-3-[(2-hydroxyethyl)sulfanyl] pyridazin-4-yl]amino}pyrimidin-4-yl)-3-(4-cyclopropylpiperazin-1-yl)cyclobutane-1-carboxamide;
N-(6-{[6-(5-chloro-2-fluorophenyl)-3-[(2-hydroxyethyl)sulfanyl]pyridazin-4-yl]amino}pyrimidin-4-yl)-3-[(4-methylpiperazin-1-yl)methyl]bicyclo[1.1.1]pentane-1-carboxamide;
Trans N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)cyclobutane-1-carboxamide;
Cis N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)cyclobutane-1-carboxamide;
N-(6-{[6-(5-chloro-2-fluorophenyl)-3-{[2-(trimethylsilyl)ethyl]sulfanyl}pyridazin-4-yl]amino}pyrimidin-4-yl)-3-(3,5-dimethylpiperazin-1-yl)propanamide;
N-(6-{[6-(5-chloro-2-fluorophenyl)-3-{methyl[(3-methyl-2-oxooxolan-3-yl)methyl]amino}pyridazin-4-yl]amino}pyrimidin-4-yl)-3-(3,5-dimethylpiperazin-1-yl)propanamide; and
N-(6-{[6-(5-chloro-2-fluorophenyl)-3-{methyl[(3-methyl-2-oxooxolan-3-yl)methyl]amino}pyridazin-4-yl]amino}pyrimidin-4-yl)-2-(4-methyl-1,4-diazepan-1-yl)acetamide.

11. A pharmaceutical composition comprising the compound or salt according to claim 1, in admixture with one or more pharmaceutically acceptable carriers or excipients.

12. The pharmaceutical composition according to claim 11, wherein the composition is formulated for administration by inhalation.

13. (canceled)

14. A method for treating a disease, disorder, or condition mediated by an ALK5 signaling pathway, comprising administering the pharmaceutical composition according to claim 11 to a mammal in need of such treatment.

15. A method of treating at least one selected from the group consisting of fibrosis and a disease, disorder, or condition that involves fibrosis, comprising administering the pharmaceutical composition according to claim 11 to a subject in need of such treatment.

16. A method of treating at least one selected from the group consisting of pulmonary fibrosis, idiopathic pulmonary fibrosis (IPF), hepatic fibrosis, renal fibrosis, ocular fibrosis, cardiac fibrosis, arterial fibrosis, and systemic sclerosis, comprising administering the pharmaceutical composition according to claim 11 to a subject in need of such treatment.

17. A method of treating idiopathic pulmonary fibrosis (IPF), comprising administering the pharmaceutical composition according to claim 11 to a subject in need of such treatment.
